(19) United States
(12) Patent Application Publication
(10) Pub. No.: US 2025/0215012 A1
(43) Pub. Date: Jul. 3, 2025

(54) IKZF2 DEGRADERS AND USES THEREOF

(71) Applicants: REGENTS OF THE UNIVERSITY OF MICHIGAN, Ann Arbor, MI (US); Oncopia Therapeutics, Inc. d/b/a SK Life Science Labs, King Of Prussia, PA (US)

(72) Inventors: Zhixiang Chen, Ann Arbor, MI (US); Rohan Rej, Ann Arbor, MI (US); Xuqing Zhang, NEW YORK, NY (US); Longchuan Bai, Ann Arbor, MI (US); Paul Kirchhoff, Saline, MI (US); Shaomeng Wang, Superior Township, MI (US)

(21) Appl. No.: 18/849,594

(22) PCT Filed: Mar. 24, 2023
(86) PCT No.: PCT/US2023/016177

§ 371 Date: Sep. 23, 2024

Related U.S. Application Data
(60) Provisional application No. 63/446,105, filed on Feb. 16, 2023.
(60) Provisional application No. 63/323,792, filed on Mar. 25, 2022.

(51) Int. Cl. C07D 491/20; A61K 31/4545; C07D 471/20; C07D 519/00

(52) U.S. Cl. CPC C07D 491/20; A61K 31/4545; C07D 471/20; C07D 519/00

(57) ABSTRACT

Described herein are compounds of Formulae I′ and their pharmaceutically acceptable salts, solvates, or stereoisomers, as well as their uses (e.g., as IKZF2 degraders).

### RELATED APPLICATIONS

This application claims the benefit of and priority to U.S. Provisional Application No. 63/446,105, filed Feb. 16, 2023, and U.S. Provisional Application No. 63/323,792, filed Mar. 25, 2022, the contents of each of which are incorporated herein by reference in their entireties.

### BACKGROUND

Described herein IKAROS Family Zinc Finger 2 (IKZF2) (also known as Helios) is one of the five members of the Ikaros family of transcription factors found in mammals. IKZF2 contains four zinc finger domains near the N-terminus, which are involved in DNA binding, and two zinc finger domains at the C-terminus, which are involved in protein dimerization. IKZF2 is about 50% identical with Ikaros family members, Ikaros (IKZF1), Aiolos (IKZF3), and Eos (IKZF4) with highest homology in the zinc finger regions (80%+identity). These four Ikaros family transcription factors bind to the same DNA consensus site and can heterodimerize with each other when co-expressed in cells. The fifth Ikaros family protein, Pegasus (IKZF5), is only 25% identical to IKZF2, binds a different DNA site than other Ikaros family members and does not readily heterodimerize with the other Ikaros family proteins. IKZF2, IKZF1 and IKZF3 are expressed mainly in hematopoietic cells while IKZF4 and IKZF5 are expressed in a wide variety of tissues.

IKZF2 is a critical regulator of T cell activity and function. Genetic deletion of Helios resulted in an enhanced anti-tumor immune response. Notably, Helios is highly expressed in regulatory T cells, a subpopulation of T cells that restricts the activity of effector T cells. Selective deletion of Helios in regulatory T cells resulted in both loss of suppressive activity and acquisition of effector T cell functions. Therefore, Helios is a critical factor in restricting T cell effector function in T_{regs}. Currently, anti-CTLA4 antibodies are used in the clinic to target T_{regs} in tumors. However, targeting CTLA4 often causes systemic activation of T-effector cells, resulting in excessive toxicity and limiting therapeutic utility. Up to ¾ of patients treated with a combination of anti-PD-1 and anti-CTLA4 have reported grade 3 or higher adverse events. Thus, a strong need exists to provide compounds that target T_{regs} in tumors without causing systemic activation of T-effector cells. An IKZF2-specific degrader has the potential to focus the enhanced immune response to areas within or near tumors providing a potentially more tolerable and less toxic therapeutic agent for the treatment of cancer.

Helios expression has also been reported to be upregulated in ‘exhausted’ T cells, in the settings of both chronic viral infections, as well as in dysfunctional chimeric antigen receptor (CAR) T cells. Overexpression or aberrant expression of Helios and various splice isoforms have been reported in several hematological malignancies, including T cell leukemias and lymphomas. Moreover, knockdown of Helios in a model of mixed lineage leukemia (MLL)-driven myeloid leukemia potently suppressed proliferation and increased cell death. In line with these results, genomic profiling and chromatin accessibility analysis demonstrated that IKZF2 loss led to increased myeloid differentiation. These data suggest that IKZF2 is differentially required in myeloid leukemia cells compared to normal cells. Therefore, depletion of IKZF2 has preferential effect in leukemic stem cells compared to normal hematopoietic stem cells, providing a new strategy for targeting leukemic stem cells.

### SUMMARY

The present disclosure provides compounds of Formula (P):

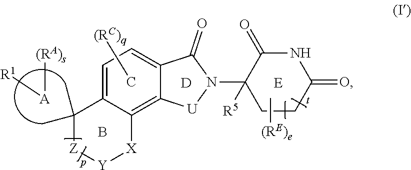

wherein each of the variables in Formula I′, is described, embodied, and exemplified herein.

In certain aspects, the present disclosure provides pharmaceutical compositions comprising a compound disclosed herein, and a pharmaceutically acceptable excipient.

In certain aspects, the present disclosure further provides methods of degrading an IKZF2 protein in a subject or biological sample comprising administering a compound disclosed herein to the subject or contacting the biological sample with a compound disclosed herein.

In certain aspects, the present disclosure further provides uses of a compound disclose herein in the manufacture of a medicament for degrading an IKZF2 protein in a subject or biological sample.

In certain aspects, the present disclosure provides compounds disclosed herein for use in degrading an IKZF2 protein in a subject or biological sample.

In certain aspects, the present disclosure provides methods of treating a disease or disorder comprising administering to a subject in need thereof a compound disclosed herein.

In certain aspects, the present disclosure provides uses of a compound disclosed herein in the manufacture of a medicament for treating a disease or disorder.

In certain aspects, the present disclosure provides compounds disclosed herein for use in treating a disease or disorder.

In certain aspects, the present disclosure provides methods of (a) increasing IL-2 production; (b) suppressing regulatory T cells; (c) enhancing effector T cells; (d) inhibiting tumor growth; and/or (e) enhancing tumor regression in a subject, comprising administering to the subject in need thereof a compound disclosed herein.

In certain aspects, the present disclosure provides use of a compound disclosed herein in the manufacture of a medicament for (a) increasing IL-2 production; (b) suppressing regulatory T cells; (c) enhancing effector T cells; (d) inhibiting tumor growth; and/or (e) enhancing tumor regression in a subject.

### DETAILED DESCRIPTION

The present disclosure relates to compounds and methods of degrading a IKZF2 protein comprising contacting a IKZF2 protein with a therapeutically effective amount of a IKZF2 degrader. The invention also relates to methods of treating a IKZF2 protein-mediated disease or condition in a patient by administering a therapeutically effective amount of a IKZF2 degrader to a patient in need thereof. The invention further relates to methods of treating a IKZF2-mediated disease or condition in a patient, the method comprising administering a pharmaceutical composition comprising a therapeutically effective amount of a IKZF2 degrader to a patient in need thereof.

### Compounds of the Preset Disclosure

In certain aspects, the present disclosure provides compounds of Formula (I′):

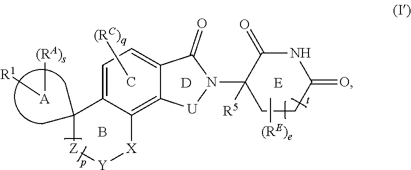

and pharmaceutically acceptable salts, solvates, or stereoisomrs thereof, wherein:
X is —C(R³)₂—, —NR⁴—, —O—, —S—, —S(═O)—, or —S(═O)₂—;
Y is —C(R³)₂—, —NR⁴—, —O—, —S—, —S(═O)—, or —S(═O)₂—;
each Z is independently —C(R³)₂—, —NR⁴—, —O—, —S(═O)—, or —S(═O)₂—;
p is 0, 1, or 2;
each R³ is independently deuterium, hydrogen, halogen, —CN, —NO₂, —OH, —NH₂, C₁₋₆alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R, —NR^{c}S(═O)₂OR^{b}, —NR^{c}S(═O)₂NR^{c}R^{d}, NR^{b}C(═O)NR^{c}R^{d}, —NR^{b}C(═O)R, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d} wherein the alkyl, alkoxy, alylamino, alkenyl, alkynyl, aryl, heteroaryl, carbocyclyl, or heterocyclylis optionally substituted with one or more R^{u};
two geminal R³ together form oxo; or
two geminal R³, together with the carbon atom to which they are attached, form C₃₋₆ carbocyclyl or 3- to 6-membered heterocyclyl, wherein the carbocyclyl or heterocyclyl is optionally substituted with one or more R^{u};
each R⁴ is independently hydrogen or C₁₋₆ alkyl optionally substituted with one or more R^{u};
Ring A is C₃₋₁₂carbocycle or 3- to 12-membered heterocycle;
R¹ is hydrogen or -M-L-Q-R²;
M is absent, —(C═O)—, —S(═O)—, or —S(═O)₂—;
L is absent or [W]ᵣ;
r is an integer from 1 to 3;
each W is independently —C(R^{L})₂—, C₃₋₄ carbocyclylene, or 3- to 4-membered heterocyclylene, wherein the carbocyclylene or heterocyclylene is optionally substituted with one or more R^{u};
each R^{L} is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂carbocyclyl, or 3- to 12-membered heterocyclyl, wherein the alkyl, alkoxy, alkylamino, alkenyl, alkynyl, aryl, heteroaryl carbocyclyl, or heterocyclyl is optionally substituted with one or more R^{u}; or
two geminal R^{L}, together with the carbon atom to which they are attached, form C₃₋₆ carbocyclyl or 3- to 6-membered heterocyclyl, wherein the carbocyclyl or heterocyclyl is optionally substituted with one or more R¹;
Q is absent, —NR^{Q}—, —O—, —C(═O)—, —S(═O)—, or —S(═O)₂—;
R^{Q} is hydrogen or C₁₋₆ alkyl optionally substituted with one or more R^{u};
R² is C₆₋₁₀ aryl, 5- to 10-membered heteroaryl, C₃₋₁₂ carbocyclyl, or 3- to 12-membered heterocyclyl, wherein the aryl, heteroaryl, carbocyclyl, or heterocyclyl is optionally substituted with one or more R^{2a};
each R^{2a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkyl)-(C₆₋₁₀ aryl), —(C₁₋₆ alkyl)-(5- to 10-membered heteroaryl), —(C₁₋₆ alkyl)-(C₃₋₁₂ carbocyclyl), —(C₁₋₆ alkyl)-(3- to 12-membered heterocyclyl), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(O)₂NR^{C}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{b}, wherein the alkyl, alkoxy, alkylamino, alkenyl, alkynyl, aryl, heteroaryl, carbocyclyl, or heterocyclyl is optionally substituted with one or more R^{u}; or
two R^{2a}, together with the atoms to which they are bonded, form C₃₋₈ carbocyclyl or 3- to 8-membered heterocyclyl, wherein the carbocyclyl or heterocyclyl is optionally substituted with one or more R^{u};
each occurrence of R^{A}, R^{C}, and R^{E} is independently oxo, halogen, —CN, —NO₂, —OH, —NH₂, C₁₋₆ alkyl, C₁₋₆-alkoxy, C₁₋₆ alkylamino, C₂₋₆alkenyl, C₂₋₆alkynyl, C₆₋₁₀ aryl, 5- to 10-membered heteroaryl, C₃₋₁₂carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkenyl, carbocyclyl, heterocyclyl, aryl, or heteroaryl is optionally substituted with one or more R^{u};
q is an integer from 0 to 2;
s is an integer from 0 to 12, as valency permits;
e is an integer selected from 0 to 5;
U is —CH₂— or —C(═O)—;
R⁵ is hydrogen, deuterium, C₁₋₆ haloalkyl, or C₁₋₆ alkyl; and
t is an integer from 0 to 2;
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆alkylene)-(C₆₋₁₀ aryl), —(C₁₋₆ alkylene)-(5- to 10-membered heteroaryl), —(C₁₋₆ alkylene)-(C₃₋₁₂carbocyclyl), —(C₁₋₆ alkylene)-(3- to 12-membered heterocyclyl), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═)NR^{c}R^{d}, —NR^{b}C(═O)R^{a}, —NR^{b}C(═O)OR^{b}, —OS(═O)₂R^{a}, —OS(═O)₂OR^{b}, —OS(═NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkylene, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂ alkenyl, C₂₋₆ alkynyl, C₆₋₁₀ aryl, 5- to 10-membered heteroaryl, C₃₋₁₂carbocyclyl, and 3- to 12-membered heterocyclyl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
R^{c} and R^{d} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl, wherein the heterocyclyl is optionally substituted with one or more R^{z},
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the compound is a compound of Formula (I′-1-i), (I′-1-ii), (I′-1-iii), (I′-1-iv), (I′-1-v), (I′-1-vi), (I′-1-vii), (I′-1-viii), (I′-1-ix), (I′-1-x), (I′-1-xi), (I′-1-xii), or (I′-1-xiii):

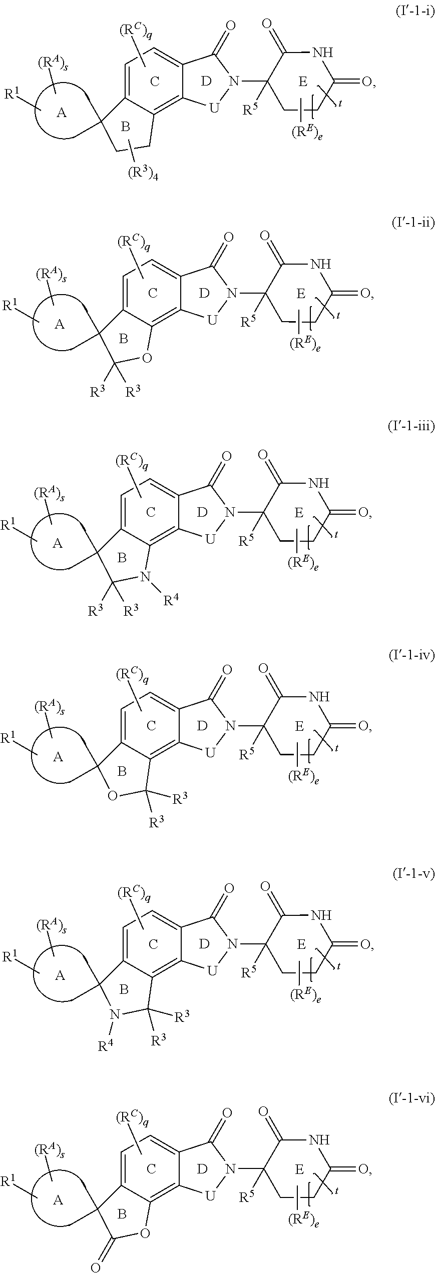

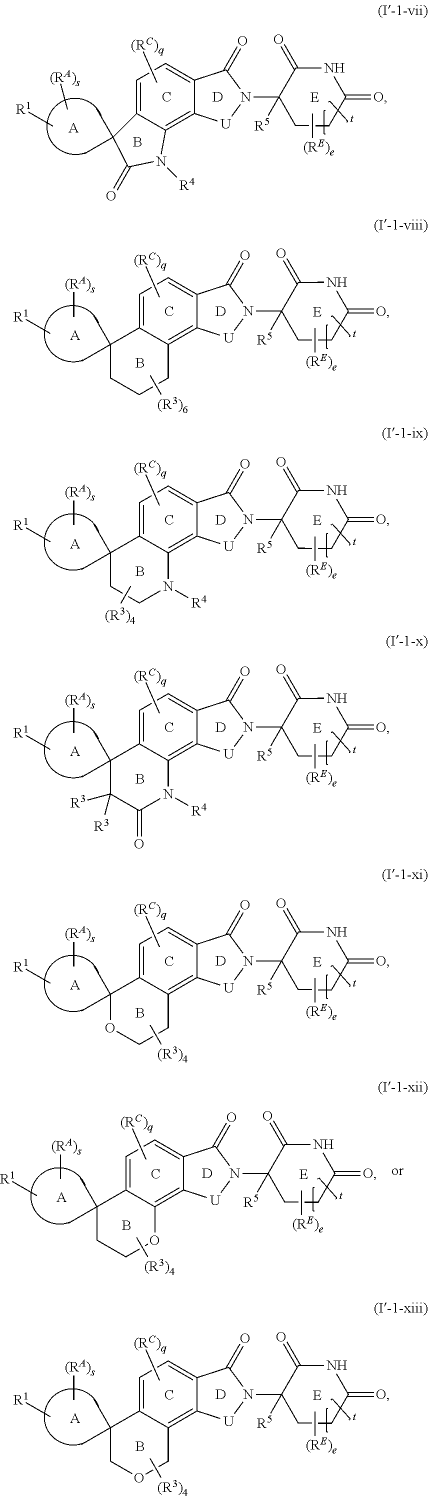

or a pharmaceutically acceptable salt, solvate, or stereoisomer thereof.

In certain embodiments, the compound is a compound of Formula (I′-2-i), (I′-2-ii), (I′-2-iii), (I′-2-iv), (I′-2-v), (I′-2-vi), (I′-2-vii), (I′-2-viii), (I′-2-ix), (I′-2-x), (I′-2-xi), (I′-2-xii), or (I′-2-xii):

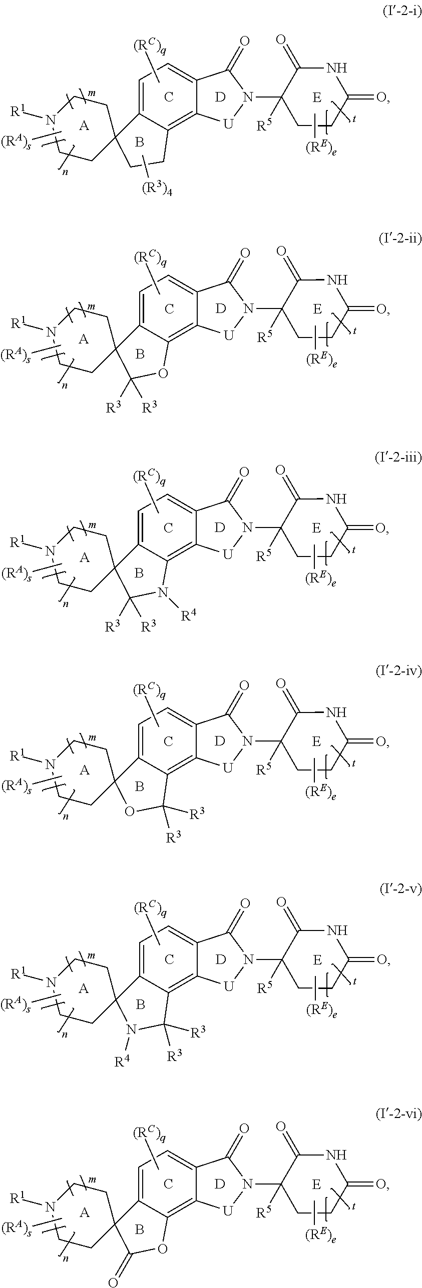

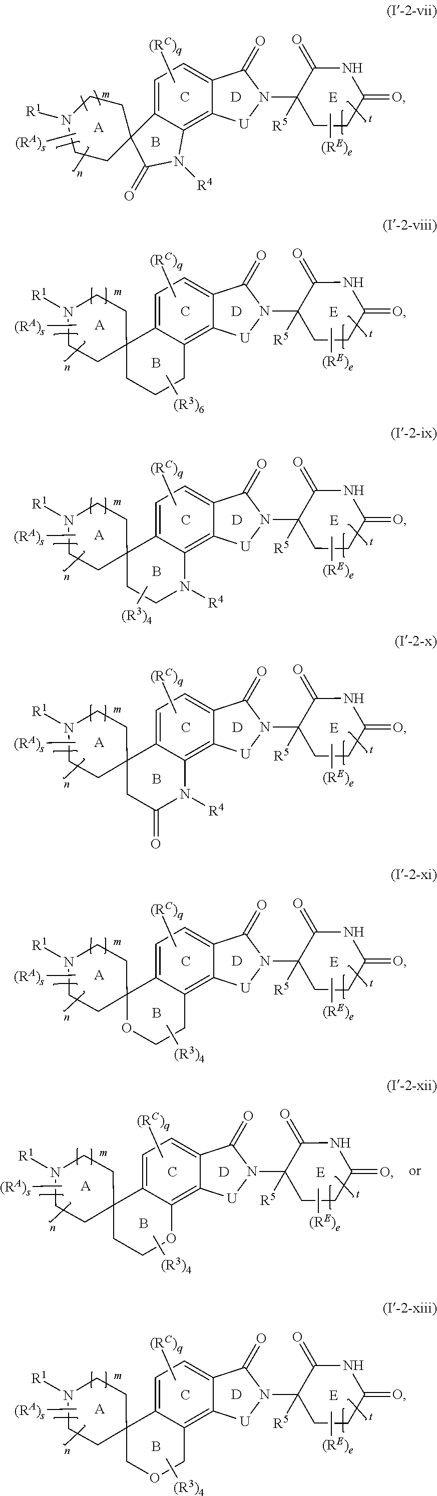

or a pharmaceutically acceptable salt, solvate, or stereoisomer thereof.

In certain embodiments, when p is 0, then X and Y are not both —C(R³)₂; and/or when p is 1, then X, Y, and Z are not all —C(R³)₂.

In certain embodiments, Ring A is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) or 3- to 12-membered heterocyclyl (e.g., heterocyclyl comprising one or two 3- to 8-membered rings and 1-5 heteroatoms selected from N, O, and S).

In certain embodiments,

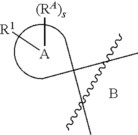

is

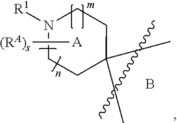

wherein m and n are independently an integer from 0 to 2.

In certain embodiments, m is 0. In certain embodiments, m is 1. In certain embodiments, m is 2. In certain embodiments, n is 0. In certain embodiments, n is 1. In certain embodiments, nis 2.

In certain embodiments, each of m and n is 1.

In certain embodiments, R¹ is hydrogen or -M-L-Q-R².

In certain embodiments, M is absent, —(C═O)—, —S(═O)—, or —S(═O)₂—.

In certain embodiments, L is absent or [W]ᵣ.

In certain embodiments, each W is independently —C(R^{L})₂—, C₃₋₄ carbocyclylene (e.g., cyclopropylene (C₃), cyclopropenylene (C₃), cyclobutylene (C₄), or cyclobutenylene (C₄)), or 3- to 4-membered heterocyclylene (e.g., heterocyclylene comprising one 3- to 4-membered rings and 1 heteroatom selected from N, O, and S), wherein the carbocyclylene or heterocyclylene is optionally substituted with one or more R^{u}.

In certain embodiments, each R^{L} is independently hydrogen, deuterium, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄),s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl(C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{L} is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{L} is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{L} is independently hydrogen, deuterium, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{L} is independently hydrogen, deuterium, or C₁₋₆ alkyl.

In certain embodiments, L is —CH₂—.

In certain embodiments, two geminal R^{L}, together with the carbon atom to which they are attached, form C₃₋₆ carbocyclyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one or two 3- to 6-membered rings and 1-3 heteroatoms selected from N, O, and S), wherein the carbocyclyl or heterocyclyl is optionally substituted with one or more R^{u}.

In certain embodiments, r is an integer from 1 to 3. In certain embodiments, r is 1. In certain embodiments, r is 1. In certain embodiments, r is 2. In certain embodiments, r is 3.

In certain embodiments, Q is absent, —NR^{Q}—, —O—, —C(═O)—, —S(═O)—, or —S(═O)₂—.

In certain embodiments, R^{Q} is hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, R² is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the aryl, heteroaryl, carbocyclyl, or heterocyclyl is optionally substituted with one or more R^{2a}.

In certain embodiments, each R^{2a} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkylene, alkoxy, alkylamino, alkenyl, alkynyl, aryl, heteroaryl, carbocyclyl, or heterocyclyl is optionally substituted with one or more R^{u}.

In certain embodiments, each R^{2a} is independently oxo, halogen, —CN, —OH, C₁₋₆ alkyl, C₁₋₆ alkoxy, C₂₋₆ alkylamino, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂NR^{c}R^{d}, —NR^{c}S(═O)₂R^{a}, —NR^{b}C(═O)R^{a}, —C(═O)OR^{b}, or —C(═O)NR^{c}R^{d}, wherein the alkyl, alkylene, alkoxy, alkylamino, alkenyl, alkynyl, carbocyclyl, heterocyclyl, aryl, or heteroaryl is optionally substituted with one or more R^{u}.

In certain embodiments, two R^{2a}, together with the atoms to which they are bonded, form C₃₋₈ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈)) or 3- to 8-membered heterocyclyl (e.g., heterocyclyl comprising one or two 3- to 8-membered rings and 1-3 heteroatoms selected from N, O, and S), wherein the carbocyclyl or heterocyclyl is optionally substituted with one or more R^{u}.

In certain embodiments, each occurrence of R^{A}, R^{C}, and R^{E} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{A}, R^{C}, and R^{E} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{A}, R^{C}, and R^{E} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each occurrence of R^{A}, R^{C}, and R^{E} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, q is an integer from 0 to 2. In certain embodiments, q is 0. In certain embodiments, q is 1. In certain embodiments, q is 2.

In certain embodiments, s is an integer from 0 to 12, as valency permits. In certain embodiments, s is 0. In certain embodiments, s is 1. In certain embodiments, s is 2. In certain embodiments, s is 3. In certain embodiments, s is 4. In certain embodiments, s is 5, as valency permits. In certain embodiments, s is 6, as valency permits. In certain embodiments, s is 7, as valency permits. In certain embodiments, s is 8, as valency permits. In certain embodiments, s is 9, as valency permits. In certain embodiments, s is 10, as valency permits. In certain embodiments, s is 11, as valency permits. In certain embodiments, s is 12, as valency permits.

In certain embodiments, e is an integer selected from 0 to 5. In certain embodiments, e is 0. In certain embodiments, e is 1. In certain embodiments, e is 2. In certain embodiments, e is 3. In certain embodiments, e is 4. In certain embodiments, e is 5.

In certain embodiments, X is —C(R³)₂—, —NR⁴—, —O—, —S—, —S(═O)—, or —S(═O)₂—.

In certain embodiments, Y is —C(R³)₂—, —NR⁴—, —O—, —S—, —S(═O)—, or —S(═O)₂—.

In certain embodiments, each Z is independently —C(R³)₂—, —NR⁴—, —O—, —S—, —S(═O)—, or —S(═O)₂—.

In certain embodiments, X is —O— and Y is —C(R³)₂—. In certain embodiments, X is —C(R³)₂— and Y is —O—. In certain embodiments, X is —NR⁴— and Y is —C(R³)₂—.

In certain embodiments, p is 0, 1, or 2. In certain embodiments, p is 0. In certain embodiments, p is 1. In certain embodiments, p is 2.

In certain embodiments, each R³ is independently deuterium, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, aryl, heteroaryl, carbocyclyl, or heterocyclylis optionally substituted with one or more R^{u}.

In certain embodiments, each R³ is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R³ is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R³ is independently hydrogen, deuterium, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R³ is independently hydrogen, deuterium, or C₁₋₆ alkyl.

In certain embodiments, two geminal R³ together form oxo.

In certain embodiments, two geminal R³, together with the carbon atom to which they are attached, form C₃₋₆ carbocyclyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one or two 3- to 6-membered rings and 1-3 heteroatoms selected from N, O, and S), wherein the carbocyclyl or heterocyclyl is optionally substituted with one or more R^{u}.

In certain embodiments, each R⁴ is independently hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, U is —CH₂— or —C(═O)—.

In certain embodiments, R⁵ is hydrogen, deuterium, C₁₋₆ haloalkyl (e.g., C₁₋₆ alkyl substituted by 1 to 8 halogen atoms selected from —F, —Cl, —Br, or —I), or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C⁵), or hexyl (C₆)).

In certain embodiments, t is an integer from 0 to 2. In certain embodiments, t is 0. In certain embodiments, t is 1. In certain embodiments, t is 2.

In certain embodiments, the compound is a compound of Formula (I′):

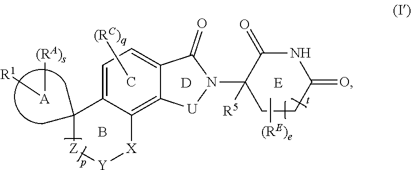

or a pharmaceutically acceptable salt, solvate, or stereoisomer thereof, wherein:
R¹ is hydrogen or -L-R²;
L is —C(R^{L})₂—;
each R^{L} is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u};
R² is C₆₋₁₀ aryl, 5- to 10-membered heteroaryl, C₃₋₁₂ carbocyclyl, or 3- to 12-membered heterocyclyl, wherein the aryl, heteroaryl, carbocyclyl, or heterocyclyl is optionally substituted with one or more R^{2a};
each R^{2a} is independently oxo, halogen, —CN, —OH, C₁₋₆ alkyl, C₁₋₆ alkoxy, C₂₋₆ alkylamino, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂NR^{c}R^{d}, —NR^{c}S(═O)₂R^{a}, —NR^{b}C(═O)R^{a}, —C(═O)OR^{b}, or —C(═O)NR^{c}R^{d}, wherein the alkyl, alkylene, alkoxy, alkylamino, alkenyl, alkynyl, carbocyclyl, heterocyclyl, aryl, or heteroaryl is optionally substituted with one or more R^{u};
each occurrence of R^{A}, R^{C}, and R^{E} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u};
X is —O— or —NR⁴—;
each R⁴ is independently hydrogen or C₁₋₆ alkyl;
Y is —CH₂— or —O—; and
p is 0 or 1.

In certain aspects, the present disclosure provides compounds of Formula (I):

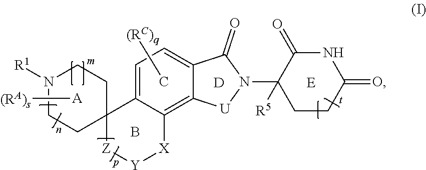

and pharmaceutically acceptable salts, solvates, or stereoisomers thereof, wherein:
R¹ is hydrogen or -M-L-Q-R²;
M is absent, —(C═O)—, —S(═O)—, or —S(═O)₂—;
L is absent or [—C(R^{L})₂—]ᵣ;
each R^{L} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, or 3- to 6-membered heterocyclyl, wherein the alkyl, alkoxy, alkylamino, alkenyl, alkynyl, carbocyclyl, heterocyclyl, aryl, or heteroaryl is optionally substituted with one or more R^{u}; or
two R^{L}, together with the carbon atom(s) to which they are attached, form C₃₋₁₂ carbocyclyl or 3- to 12-membered heterocyclyl;
r is an integer from 1 to 3;
Q is absent, —NR^{Q}—, —O—, —C(═O)—, —S(═O)—, or —S(═O)₂—;
R^{Q} is hydrogen, C₁₋₆ alkyl, wherein the alkyl is optionally substituted with one or more R^{u};
R² is C₆₋₁₀ aryl, 5- to 10-membered heteroaryl, C₃₋₁₂ carbocyclyl, or 3- to 12-membered heterocyclyl, wherein the aryl, heteroaryl, carbocyclyl, or heterocyclyl is optionally substituted with one or more R^{2a};
each R^{2a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkyl)-(C₆₋₁₀ aryl), —(C₁₋₆ alkyl)-(5- to 10-membered heteroaryl), —(C₁₋₆ alkyl)-(C₃₋₁₂ carbocyclyl), —(C₁₋₆ alkyl)-(3- to 12-membered heterocyclyl), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or two R^{2a} together form oxo;
each occurrence of R^{A} and R^{C} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
q is an integer from 0 to 2;
s is an integer from 0 to 12, as valency permits;
m and n are independently an integer from 0 to 2;
X is —C(R³)₂—, —NR⁴—, —O—, —S—, —S(═O)—, or —S(═O)₂—;
Y is —C(R³)₂—, —NR⁴—, —O—, —S—, —S(═O)—, or —S(═O)₂—;
each Z is independently —C(R³)₂—, —NR⁴—, —O—, —S—, —S(═O)—, or —S(═O)₂—;
p is 0 or 1;
each R³ is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R³ together form oxo; or
two R³, together with the carbon atom(s) to which they are attached, form C₃₋₁₂ carbocyclyl or 3- to 12-membered heterocyclyl;
each R⁴ is independently hydrogen or C₁₋₆ alkyl, wherein the alkyl is optionally substituted with one or more R^{u};
U is —CH₂— or —C(═O)—;
R⁵ is hydrogen, deuterium, C₁₋₆ haloalkyl, or C₁₋₆ alkyl; and
t is an integer from 0 to 2;
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkyl)-(C₆₋₁₀ aryl), —(C₁₋₆ alkyl)-(5- to 10-membered heteroaryl), —(C₁₋₆ alkyl)-(C₃₋₁₂ carbocyclyl), —(C₁₋₆ alkyl)-(3- to 12-membered heterocyclyl), —SR^{b}, —S(═O)R^{a}, —S(═O)₂R^{a}, —S(═O)₂OR^{b}, —S(—O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₁₂ carbocyclyl, and 3- to 6-membered heterocyclyl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl.

In certain embodiments, X is —C(R³)₂—.

In certain embodiments, X is —NR⁴—.

In certain embodiments, X is —O—.

In certain embodiments, X is —S—.

In certain embodiments, X is —S(═O)—.

In certain embodiments, X is —S(═O)₂—.

In certain embodiments, Y is —C(R³)₂—.

In certain embodiments, Y is —NR⁴—.

In certain embodiments, Y is —O—.

In certain embodiments, Y is —S—.

In certain embodiments, Y is —S(═O)—.

In certain embodiments, Y is —S(═O)₂—.

In certain embodiments, X is —O— and Y is —C(R³)₂—. In some embodiments, X is —O—, and Y is —CH₂—. In certain embodiments, X is —C(R³)₂— and Y is —O—. In certain embodiments, X is —NR⁴— and Y is —C(R³)₂—. In certain embodiments, X is —C(R³)₂— and Y is —NR⁴—.

In some embodiments, X is —O—, Y is —C(R³)₂—, and p is 0.

In certain embodiments, Z is —C(R³)₂—, —NR⁴—, or —O—. In certain embodiments, Z is —C(R³)₂ or —O—.

In certain embodiments, when p is 0, then X and Y are not both —C(R³)₂; or when p is 1, then X, Y, and Z are not all —C(R³)₂.

In certain embodiments, p is 0. In certain embodiments, p is 1.

In certain embodiments, the compound is a compound of Formula (I-1-i) to (I-1-xiii):

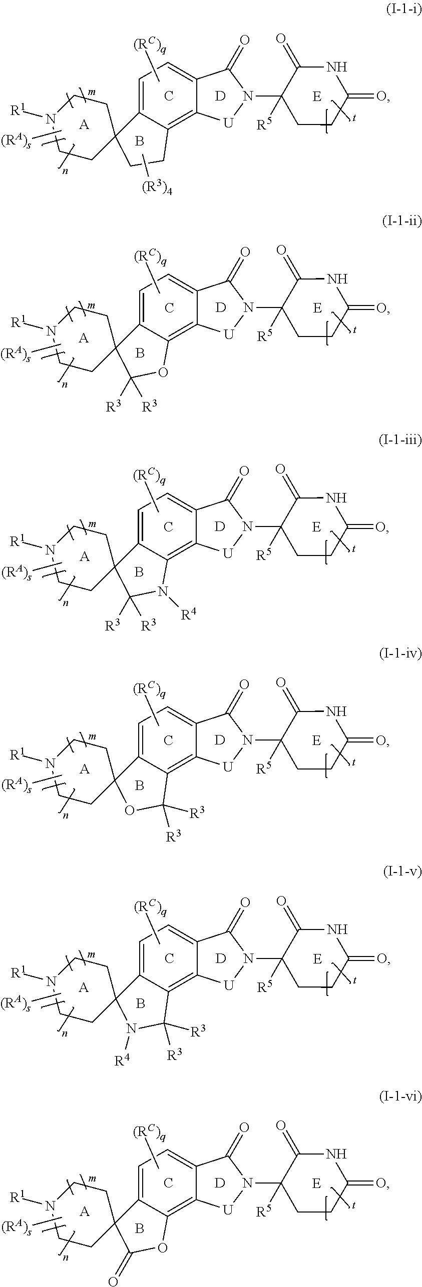

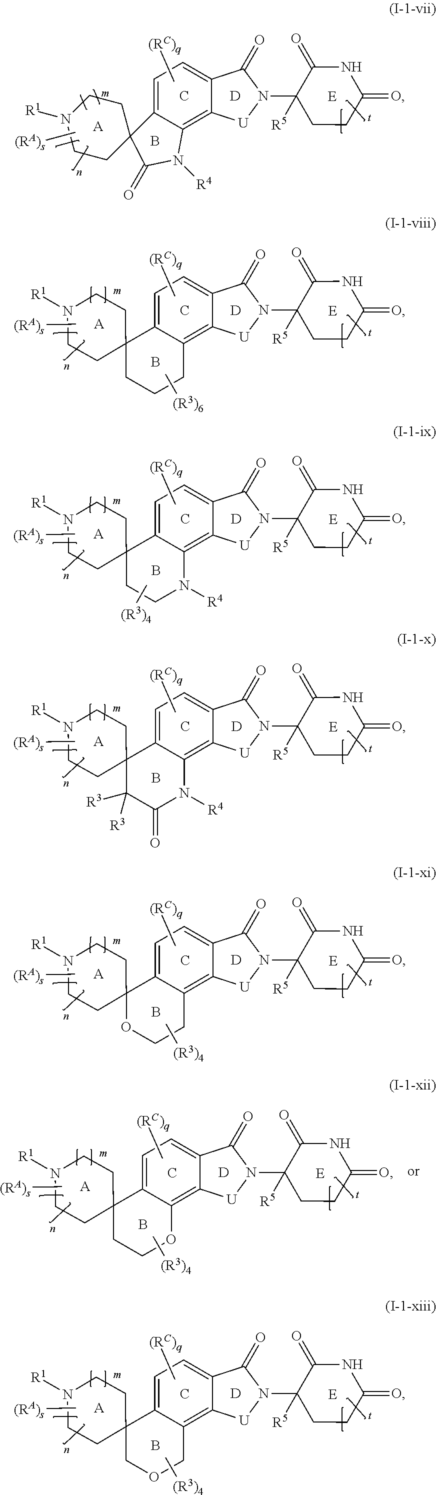

or a pharmaceutically acceptable salt, solvate, or stereoisomer thereof.

In certain embodiments, R¹ is hydrogen. In certain embodiments, R¹ is -M-L-Q-R².

In certain embodiments, M is absent. In certain embodiments, M is —(C═O)—, —S(═O)—, or —S(═O)₂—.

In certain embodiments, L is —C(R^{L})₂—. In certain embodiments, L is absent.

In certain embodiments, each R^{L} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{L} is independently hydrogen or C₁₋₆ alkyl.

In certain embodiments, L is —CH₂—.

In certain embodiments, two R^{L}, together with the carbon atom(s) to which they are attached, form C₃₋₁₂ carbocyclyl or 3- to 12-membered heterocyclyl.

In certain embodiments, Q is absent. In certain embodiments, Q is —NR^{Q}—, —O—, —C(═O)—, —S(═O)—, or —S(═O)₂—. In certain embodiments, Q is —NR^{Q}—. In certain embodiments, Q is —O—. In certain embodiments, Q is —C(═O)—. In certain embodiments, Q is —S(═O)—. In certain embodiments, Q is —S(═O)₂—.

In certain embodiments, R^{Q} is hydrogen or C₁₋₆ alkyl. In certain embodiments, R^{Q} is C₁₋₆ alkyl. In certain embodiments, R^{Q} is hydrogen.

In certain embodiments, R² is C₆₋₁₀ aryl, 5- to 10-membered heteroaryl, C₅₋₁₀ carbocyclyl, or 5- to 10-membered heterocyclyl.

In certain embodiments, R² is phenyl.

In certain embodiments, R² is 5- to 10-membered heteroaryl.

In certain embodiments, R² is C₅₋₁₀ carbocyclyl.

In certain embodiments, R² is 5- to 10-membered heterocyclyl.

In certain embodiments, each R^{2a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{2a} is independently oxo, halogen, —CN, —OH, C₁₋₆ alkyl, C₁₋₆ alkoxy, C₆₋₁₀ aryl, 5- to 10-membered heteroaryl, C₃₋₁₂ carbocyclyl, 3- to 12-membered heterocyclyl, —S(═O)₂R^{a}, —S(═O)₂NR^{c}R^{d}, —NR^{c}S(═O)₂R^{a}, —NR^{b}C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, two R^{2a} together form oxo.

In certain embodiments, each R³ is independently H or C₁₋₆ alkyl. In certain embodiments, each R³ is H. In certain embodiments, two geminal R³ together form oxo.

In certain embodiments, each R⁴ is independently hydrogen, C₁₋₆ alkyl, C₃₋₁₂ carbocyclyl, or 3- to 12-membered heterocyclyl, wherein the alkyl, carbocyclyl, or heterocyclyl is optionally substituted with one or more R^{u}. In certain embodiments, each R⁴ is independently H or C₁₋₆ alkyl, wherein the alkyl is optionally substituted with one or more R^{u}.

In certain embodiments, each occurrence of R^{A} and R^{C} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8. In certain embodiments, s is 9. In certain embodiments, s is 10. In certain embodiments, s is 11. In certain embodiments, s is 12.

In certain embodiments, q is 0. In certain embodiments, q is 1. In certain embodiments, q is 2.

In certain embodiments, m and n are independently 0 or 1. In certain embodiments, each of m and n is 0. In certain embodiments, each of m and n is 1. In certain embodiments, m is 0 and n is 1. In certain embodiments, m is 1 and n is 0.

In certain embodiments, U is —CH₂—. In certain embodiments, U is —C(═O)—.

In certain embodiments, R⁵ is hydrogen, deuterium, C₁₋₆ haloalkyl, or C₁₋₆ alkyl. In certain embodiments, R⁵ is hydrogen. In certain embodiments, R⁵ is deuterium. In certain embodiments, R⁵ is C₁₋₆ haloalkyl. In certain embodiments, R⁵ is C₁₋₆ alkyl.

In certain embodiments, t is 0. In certain embodiments, t is 1. In certain embodiments, t is 2.

In certain embodiments, the compound is a compound of Formula (I):

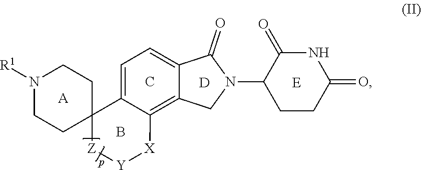

or a pharmaceutically acceptable salt, solvate, or stereoisomer thereof, wherein:
R¹ is hydrogen or -L-R²
L is —CH₂—;
R² is C₆₋₁₀ aryl, 5- to 10-membered heteroaryl, C₃₋₁₂ carbocyclyl, or 3- to 12-membered heterocyclyl, wherein the aryl, heteroaryl, carbocyclyl, or heterocyclyl is optionally substituted with one or more R^{2a};
each R^{2a} is independently oxo, halogen, —CN, —OH, C₁₋₆ alkyl, C₁₋₆ alkoxy, C₆₋₁₀ aryl, 5- to 10-membered heteroaryl, C₃₋₁₂ carbocyclyl, 3- to 12-membered heterocyclyl, —S(═O)₂R^{a}, —S(═O)₂NR^{c}R^{d}, —NR^{c}S(═O)₂R^{a}, —NR^{b}C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
X is —O—;
Y is —CH₂—; and
p is 0.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl(C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂₋₆ alkynyl, C₃₋₆ carbocyclyl, or 3- to 6-membered heterocyclyl.

In certain embodiments, each R^{b} is independently hydrogen, C₁₋₆ alkyl, C₃₋₆ carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₃₋₆ carbocyclyl, or 3- to 6-membered heterocyclyl, wherein the alkyl, carbocyclyl, or heterocyclylis optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{z}.

In certain embodiments, R^{a}, R^{b}, R^{c}, and R^{d} is independently and optionally substituted with one or more R^{z}.

In certain embodiments, R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, each R^{u} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain embodiments, two geminal R^{u}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

Embodiments of the variables in any of the Formulae described herein, e.g., Formulae I and I′, as applicable, are described below. Any of the variables can be any moiety as described in the embodiments below. In addition, the combination of any moieties described for any of the variables, as applicable, with any moieties described for any of the remaining variables, is also contemplated.

Without wishing to be limited by this statement, while various options for variables are described herein, it is understood that the present disclosure intends to encompass operable embodiments having combinations of the options. The disclosure may be interpreted as excluding the non-operable embodiments caused by certain combinations of the options. For example, while various options for variables X, Y, and Z are described herein, the disclosure may be interpreted as excluding structures for non-operable compounds caused by certain combinations of the options (e.g., when two adjacent members of X, Y, an Z are both nitrogen or both oxygen; or one of two adjacent members of X, Y, and Z is nitrogen while the other is oxygen).

When a range of values is listed, each discrete value and sub-range within the range are also contemplated. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In certain embodiments, the compound is selected from the compounds in Table 1 and pharmaceutically acceptable salts thereof.

In certain embodiments, the compound is selected from the compounds in Table 1.

TABLE 1

| No. | Structure | Compound Name |
|---|---|---|
| A1 | 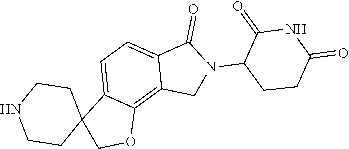 [SMILES: O=C1CCC(N2Cc3c(ccc4c3OCC43CCNCC3)C2=O)C(=O)N1] | 3-(6-oxo-6,8-dihydro-2H,7H- spiro[furo[2,3-e]isoindole-3,4′- piperidin]-7-yl)piperidine-2,6-dione |
| A2 | 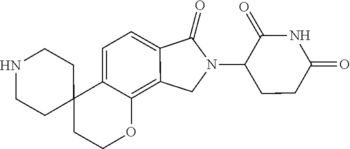 [SMILES: O=C1CCC(N2Cc3c(ccc4c3OCCC43CCNCC3)C2=O)C(=O)N1] | 3-(7′-oxo-2′,3′,7′,9′-tetrahydro-8′H- spiro[piperidine-4,4′-pyrano[2,3- e]isoindol]-8′-yl)piperidine-2,6-dione |
| A3 | 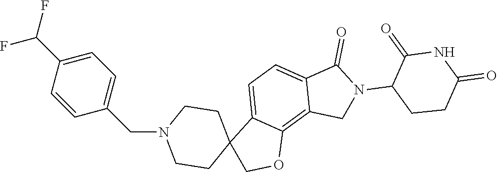 [SMILES: O=C1CCC(N2Cc3c(ccc4c3OCC43CCN(Cc4ccc(C(F)F)cc4)CC3)C2=O)C(=O)N1] | 3-(1′-(4-(difluoromethyl)benzyl)-6- oxo-6,8-dihydro-2H,7H- spiro[furo[2,3-e]isoindole-3,4′- piperidin]-7-yl)piperidine-2,6-dione |
| A4 | 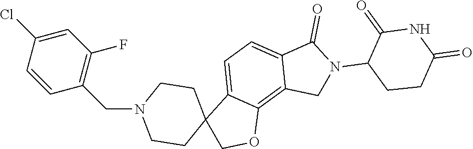 [SMILES: O=C1CCC(N2Cc3c(ccc4c3OCC43CCN(Cc4ccc(Cl)cc4F)CC3)C2=O)C(=O)N1] | 3-(1′-(4-chloro-2-fluorobenzyl)-6-oxo- 6,8-dihydro-2H,7H-spiro[furo[2,3- e]isoindole-3,4′-piperidin]-7- yl)piperidine-2,6-dione |
| A5 | 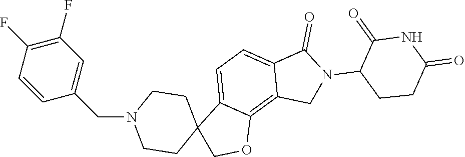 [SMILES: O=C1CCC(N2Cc3c(ccc4c3OCC43CCN(Cc4ccc(F)c(F)c4)CC3)C2=O)C(=O)N1] | 3-(1′-(3,4-difluorobenzyl)-6-oxo-6,8- dihydro-2H,7H-spiro[furo[2,3- e]isoindole-3,4′-piperidin]-7- yl)piperidine-2,6-dione |
| A6 | 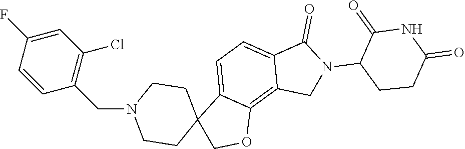 [SMILES: O=C1CCC(N2Cc3c(ccc4c3OCC43CCN(Cc4ccc(F)cc4Cl)CC3)C2=O)C(=O)N1] | 3-(1′-(2-chloro-4-fluorobenzyl)-6-oxo- 6,8-dihydro-2H,7H-spiro[furo[2,3- e]isoindoline-3,4′-piperidin]-7- yl)piperidine-2,6-dione |
| A7 | 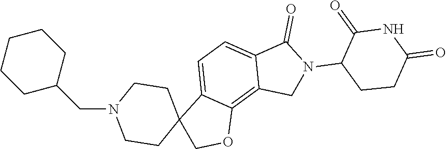 [SMILES: O=C1CCC(N2Cc3c(ccc4c3OCC43CCN(CC4CCCCC4)CC3)C2=O)C(=O)N1] | 3-(1′-(cyclohexylmethyl)-6-oxo-6,8- dihydro-2H,7H-spiro[furo[2,3- e]isoindoline-3,4′-piperidin]-7- yl)piperidine-2,6-dione |
| A8 | 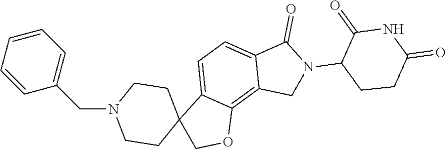 [SMILES: O=C1CCC(N2Cc3c(ccc4c3OCC43CCN(Cc4ccccc4)CC3)C2=O)C(=O)N1] | 3-(1′-benzyl-6-oxo-6,8-dihydro- 2H,7H-spiro[furo[2,3-e]isoindole-3,4′- piperidin]-7-yl)piperidine-2,6-dione |
| A9 | 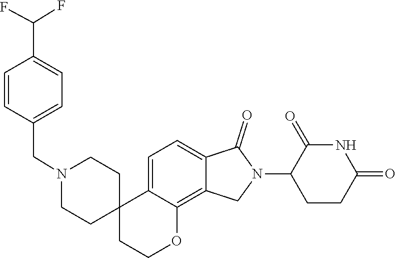 [SMILES: O=C1CCC(N2Cc3c(ccc4c3OCCC43CCN(Cc4ccc(C(F)F)cc4)CC3)C2=O)C(=O)N1] | 3-(1-(4-(difluoromethyl)benzyl)-7′- oxo-2′,3′,7′,9′-tetrahydro-8′H- spiro[piperidine-4,4′-pyrano[2,3- e]isoindol]-8′-yl)piperidine-2,6-dione |
| A10 | 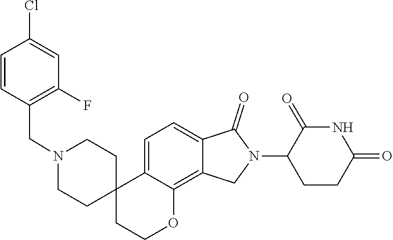 [SMILES: O=C1CCC(N2Cc3c(ccc4c3OCCC43CCN(Cc4ccc(Cl)cc4F)CC3)C2=O)C(=O)N1] | 3-(1-(4-chloro-2-fluorobenzyl)-7′-oxo- 2′,3′,7′,9′-tetrahydro-8′H- spiro[piperidine-4,4′-pyrano[2,3- e]isoindol]-8′-yl)piperidine-2,6-dione |
| A11 | 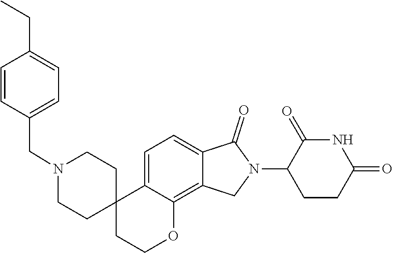 [SMILES: CCc1ccc(CN2CCC3(CCOc4c3ccc3c4CN(C4CCC(=O)NC4=O)C3=O)CC2)cc1] | 3-(1-(4-ethylbenzyl)-7′-oxo-2′,3′,7′,9′- tetrahydro-8′H-spiro[piperidine-4,4′- pyrano[2,3-e]isoindol]-8′- yl)piperidine-2,6-dione |
| A12 | 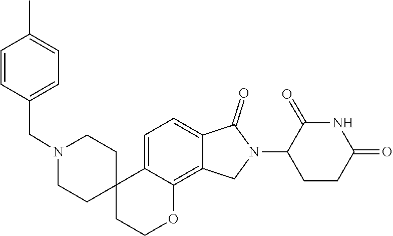 [SMILES: Cc1ccc(CN2CCC3(CCOc4c3ccc3c4CN(C4CCC(=O)NC4=O)C3=O)CC2)cc1] | 3-(1-(4-methylbenzyl)-7′-oxo- 2′,3′,7′,9′-tetrahydro-8′H- spiro[piperidine-4,4′-pyrano[2,3- e]isoindol]-8′-yl)piperidine-2,6-dione |
| A13 | 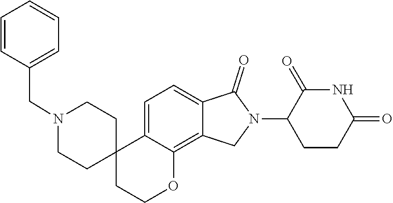 [SMILES: O=C1CCC(N2Cc3c(ccc4c3OCCC43CCN(Cc4ccccc4)CC3)C2=O)C(=O)N1] | 3-(1-benzyl-7′-oxo-2′,3′,7′,9′- tetrahydro-8′H-spiro[piperidine-4,4′- pyrano[2,3-e]isoindol]-8′- yl)piperidine-2,6-dione |
| A14 | 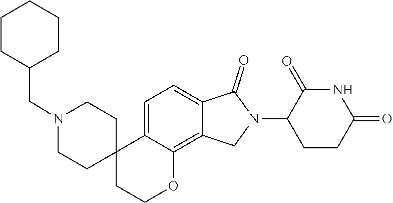 [SMILES: O=C1CCC(N2Cc3c(ccc4c3OCCC43CCN(CC4CCCCC4)CC3)C2=O)C(=O)N1] | 3-(1-(cyclohexylmethyl)-7′-oxo- 2′,3′,7′,9′-tetrahydro-8′H- spiro[piperidine-4,4′-pyrano[2,3- e]isoindol]-8′-yl)piperidine-2,6-dione |
| A15 | 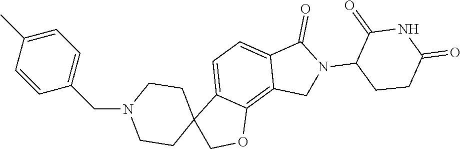 [SMILES: Cc1ccc(CN2CCC3(CC2)COc2c3ccc3c2CN(C2CCC(=O)NC2=O)C3=O)cc1] | 3-(1′-(4-methylbenzyl)-6-oxo-6,8- dihydro-2H,7H-spiro[furo[2,3- e]isoindole-3,4′-piperidin]-7- yl)piperidine-2,6-dione |
| A16 | 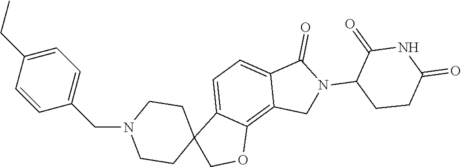 [SMILES: CCc1ccc(CN2CCC3(CC2)COc2c3ccc3c2CN(C2CCC(=O)NC2=O)C3=O)cc1] | 3-(1′-(4-ethylbenzyl)-6-oxo-6,8- dihydro-2H,7H-spiro[furo[2,3- e]isoindole-3,4′-piperidin]-7- yl)piperidine-2,6-dione |
| A17 | 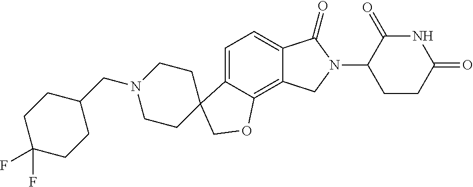 [SMILES: O=C1CCC(N2Cc3c(ccc4c3OCC43CCN(CC4CCC(F)(F)CC4)CC3)C2=O)C(=O)N1] | 3-(1′-((4,4- difluorocyclohexyl)methyl)-6-oxo- 6,8-dihydro-2H,7H-spiro[furo[2,3- e]isoindole-3,4′-piperidin]-7- yl)piperidine-2,6-dione |
| A18 | 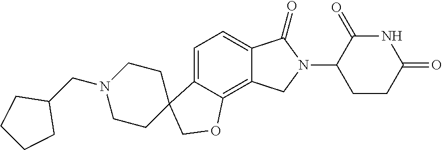 [SMILES: O=C1CCC(N2Cc3c(ccc4c3OCC43CCN(CC4CCCC4)CC3)C2=O)C(=O)N1] | 3-(1′-(cyclopentylmethyl)-6-oxo-6,8- dihydro-2H,7H-spiro[furo[2,3- e]isoindole-3,4′-piperidin]-7- yl)piperidine-2,6-dione |
| A19 | 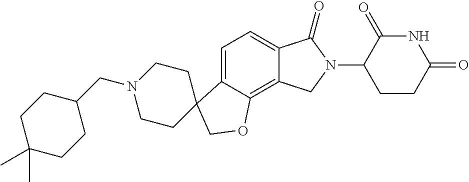 [SMILES: CC1(C)CCC(CN2CCC3(CC2)COc2c3ccc3c2CN(C2CCC(=O)NC2=O)C3=O)CC1] | 3-(1′-((4,4- dimethylcyclohexyl)methyl)-6-oxo- 6,8-dihydro-2H,7H-spiro[furo[2,3- e]isoindole-3,4′-piperidin]-7- yl)piperidine-2,6-dione |
| A20 | 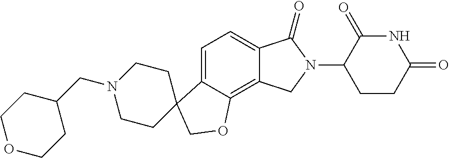 [SMILES: O=C1CCC(N2Cc3c(ccc4c3OCC43CCN(CC4CCOCC4)CC3)C2=O)C(=O)N1] | 3-(6-oxo-1′-((tetrahydro-2H-pyran-4- yl)methyl)-6,8-dihydro-2H,7H- spiro[furo[2,3-e]isoindole-3,4′- piperidin]-7-yl)piperidine-2,6-dione |
| A21 | 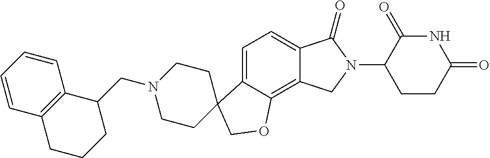 [SMILES: O=C1CCC(N2Cc3c(ccc4c3OCC43CCN(CC4CCCc5ccccc54)CC3)C2=O)C(=O)N1] | 3-(6-oxo-1′-((1,2,3,4- tetrahydronaphthalen-1-yl)methyl)- 6,8-dihydro-2H,7H-spiro[furo[2,3- e]isoindole-3,4′-piperidin]-7- yl)piperidine-2,6-dione |
| A22 | 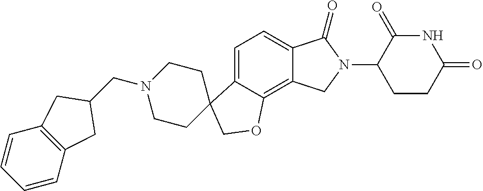 [SMILES: O=C1CCC(N2Cc3c(ccc4c3OCC43CCN(CC4Cc5ccccc5C4)CC3)C2=O)C(=O)N1] | 3-(1′-((2,3-dihydro-1H-inden-2- yl)methyl)-6-oxo-6,8-dihydro-2H,7H- spiro[furo[2,3-e]isoindole-3,4′- piperidin]-7-yl)piperidine-2,6-dione |
| A23 | 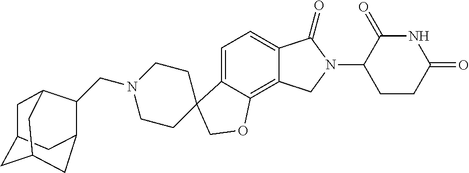 [SMILES: O=C1CCC(N2Cc3c(ccc4c3OCC43CCN(CC4C5CC6CC(C5)CC4C6)CC3)C2=O)C(=O)N1] | 3-(1′-(((1r,3r,5r,7r)-adamantan-2- yl)methyl)-6-oxo-6,8-dihydro-2H,7H- spiro[furo[2,3-e]isoindole-3,4′- piperidin]-7-yl)piperidine-2,6-dione |
| A24 | 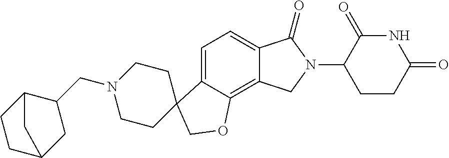 [SMILES: O=C1CCC(N2Cc3c(ccc4c3OCC43CCN(CC4CC5CCC4C5)CC3)C2=O)C(=O)N1] | 3-(1′-(bicyclo[2.2.1]heptan-2- ylmethyl)-6-oxo-6,8-dihydro-2H,7H- spiro[furo[2,3-e]isoindole-3,4′- piperidin]-7- yl)piperidine-2,6-dione |
| A25 | 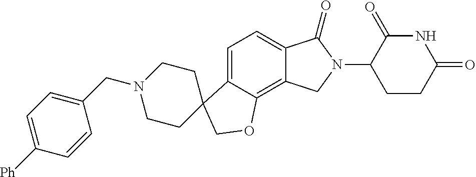 [SMILES: O=C1CCC(N2Cc3c(ccc4c3OCC43CCN(Cc4ccc(-c5ccccc5)cc4)CC3)C2=O)C(=O)N1] | 3-(1′-([1,1′-biphenyl]-4-ylmethyl)-6- oxo-6,8-dihydro-2H,7H- spiro[furo[2,3-e]isoindole-3,4′- piperidin]-7- yl)piperidine-2,6-dione |
| A26 | 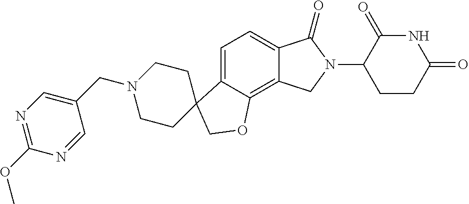 [SMILES: COc1ncc(CN2CCC3(CC2)COc2c3ccc3c2CN(C2CCC(=O)NC2=O)C3=O)cn1] | 3-(1′-((2-methoxypyrimidin-5- yl)methyl)-6-oxo-6,8-dihydro-2H,7H- spiro[furo[2,3-e]isoindole-3,4′- piperidin]-7- yl)piperidine-2,6-dione |
| A27 | 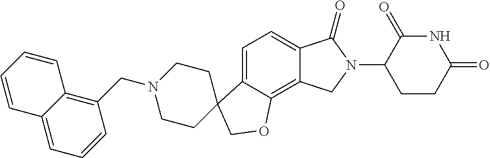 [SMILES: O=C1CCC(N2Cc3c(ccc4c3OCC43CCN(Cc4cccc5ccccc45)CC3)C2=O)C(=O)N1] | 3-(1′-(naphthalen-1-ylmethyl)-6-oxo- 6,8-dihydro-2H,7H-spiro[furo[2,3- e]isoindole-3,4′-piperidin]-7- yl)piperidine-2,6-dione |
| A28 | 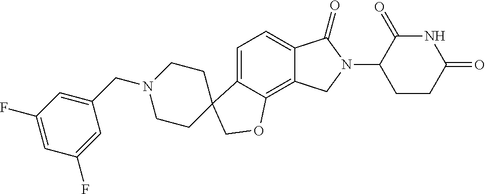 [SMILES: O=C1CCC(N2Cc3c(ccc4c3OCC43CCN(Cc4cc(F)cc(F)c4)CC3)C2=O)C(=O)N1] | 3-(1′-(3,5-difluorobenzyl)-6-oxo-6,8- dihydro-2H,7H-spiro[furo[2,3- e]isoindole-3,4′-piperidin]-7- yl)piperidine-2,6-dione |
| A29 | 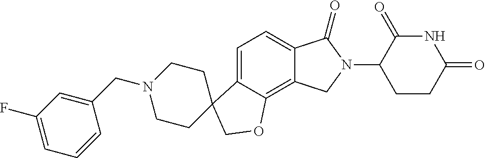 [SMILES: O=C1CCC(N2Cc3c(ccc4c3OCC43CCN(Cc4cccc(F)c4)CC3)C2=O)C(=O)N1] | 3-(1′-(3-fluorobenzyl)-6-oxo-6,8- dihydro-2H,7H-spiro[furo[2,3- e]isoindole-3,4′-piperidin]-7- yl)piperidine-2,6-dione |
| A30 | 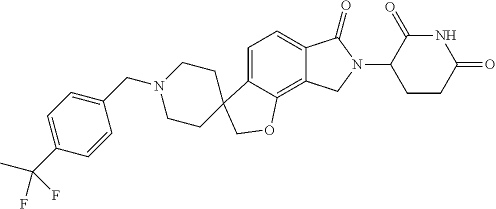 [SMILES: CC(F)(F)c1ccc(CN2CCC3(CC2)COc2c3ccc3c2CN(C2CCC(=O)NC2=O)C3=O)cc1] | 3-(1′-(4-(1,1-difluoroethyl)benzyl)-6- oxo-6,8-dihydro-2H,7H- spiro[furo[2,3-e]isoindole-3,4′- piperidin]-7- yl)piperidine-2,6-dione |
| A31 | 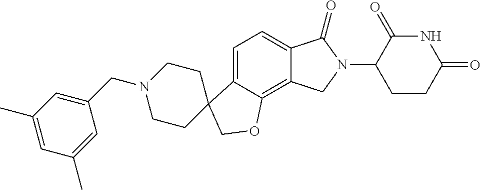 [SMILES: Cc1cc(C)cc(CN2CCC3(CC2)COc2c3ccc3c2CN(C2CCC(=O)NC2=O)C3=O)c1] | 3-(1′-(3,5-dimethylbenzyl)-6-oxo-6,8- dihydro-2H,7H-spiro[furo[2,3- e]isoindole-3,4′-piperidin]-7- yl)piperidine-2,6-dione |
| A32 | 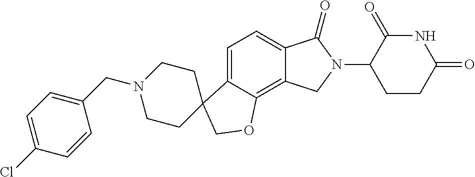 [SMILES: O=C1CCC(N2Cc3c(ccc4c3OCC43CCN(Cc4ccc(Cl)cc4)CC3)C2=O)C(=O)N1] | 3-(1′-(4-chlorobenzyl)-6-oxo-6,8- dihydro-2H,7H-spiro[2,3- e]isoindole-3,4′-piperidin]-7- yl)piperidine-2,6-dione |
| A33 | 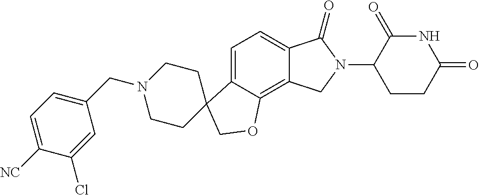 [SMILES: N#Cc1ccc(CN2CCC3(CC2)COc2c3ccc3c2CN(C2CCC(=O)NC2=O)C3=O)cc1Cl] | 2-chloro-4-((7-(2,6-dioxopiperidin-3- yl)-6-oxo-7,8-dihydro-2H,6H- spiro[furo[2,3-e]isoindole-3,4′- piperidin]-1′-yl)methyl)benzonitrile |
| A34 | 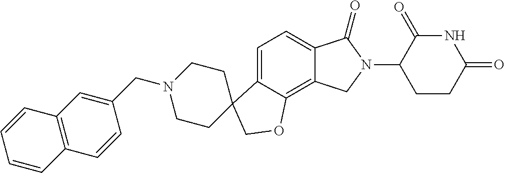 [SMILES: O=C1CCC(N2Cc3c(ccc4c3OCC43CCN(Cc4ccc5ccccc5c4)CC3)C2=O)C(=O)N1] | 3-(1′-(naphthalen-2-ylmethyl)-6-oxo- 6,8-dihydro-2H,7H-spiro[furo[2,3- e]isoindole-3,4′-piperidin]-7- yl)piperidine-2,6-dione |
| A35 | 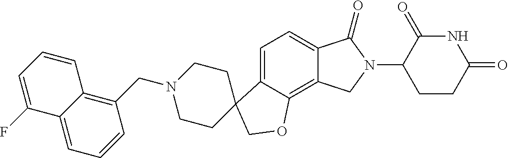 [SMILES: O=C1CCC(N2Cc3c(ccc4c3OCC43CCN(Cc4cccc5c(F)cccc45)CC3)C2=O)C(=O)N1] | 3-(1′-((5-fluoronaphthalen-1- yl)methyl)-6-oxo-6,8-dihydro-2H,7H- spiro[furo[2,3-e]isoindole-3,4′- piperidin]-7-yl)piperidine-2,6-dione |
| A36 | 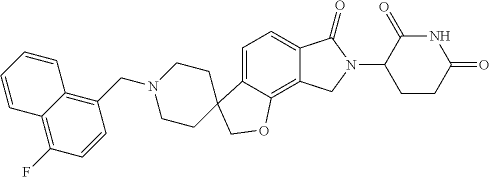 [SMILES: O=C1CCC(N2Cc3c(ccc4c3OCC43CCN(Cc4ccc(F)c5ccccc45)CC3)C2=O)C(=O)N1] | 3-(1′-((4-fluoronaphthalen-1- yl)methyl)-6-oxo-6,8-dihydro-2H,7H- spiro[furo[2,3-e]isoindole-3,4′- piperidin]-7-yl)piperidine-2,6-dione |
| A37 | 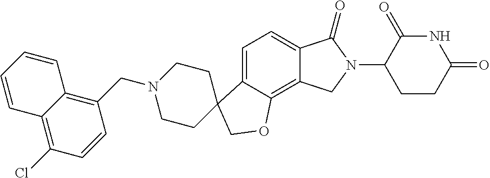 [SMILES: O=C1CCC(N2Cc3c(ccc4c3OCC43CCN(Cc4ccc(Cl)c5ccccc45)CC3)C2=O)C(=O)N1] | 3-(1′-((4-chloronaphthalen-1- yl)methyl)-6-oxo-6,8-dihydro-2H,7H- spiro[furo[2,3-e]isoindole-3,4′- piperidin]-7- yl)piperidine-2,6-dione |
| A38 | 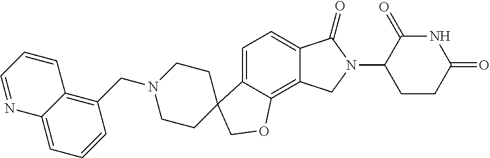 [SMILES: O=C1CCC(N2Cc3c(ccc4c3OCC43CCN(Cc4cccc5ncccc45)CC3)C2=O)C(=O)N1] | 3-(6-oxo-1′-(quinolin-5-ylmethyl)-6,8- dihydro-2H,7H-spiro[furo[2,3- e]isoindole-3,4′-piperidin]-7- yl)piperidine-2,6-dione |
| A39 | 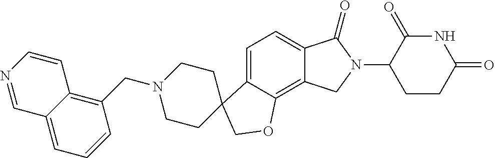 [SMILES: O=C1CCC(N2Cc3c(ccc4c3OCC43CCN(Cc4cccc5cnccc45)CC3)C2=O)C(=O)N1] | 3-(1′-(isoquinolin-5-ylmethyl)-6-oxo- 6,8-dihydro-2H,7H-spiro[furo[2,3- e]isoindole-3,4′-piperidin]-7- yl)piperidine-2,6-dione |
| A40 | 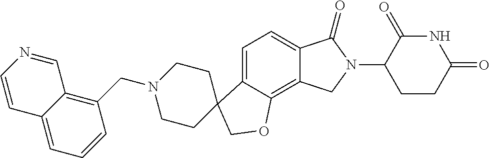 [SMILES: O=C1CCC(N2Cc3c(ccc4c3OCC43CCN(Cc4cccc5ccncc45)CC3)C2=O)C(=O)N1] | 3-(1′-(isoquinolin-8-ylmethyl)-6-oxo- 6,8-dihydro-2H,7H-spiro[furo[2,3- e]isoindole-3,4′-piperidin]-7- yl)piperidine-2,6-dione |
| A41 | 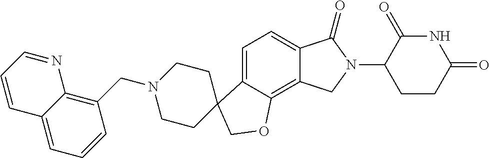 [SMILES: O=C1CCC(N2Cc3c(ccc4c3OCC43CCN(Cc4cccc5cccnc45)CC3)C2=O)C(=O)N1] | 3-(6-oxo-1′-(quinolin-8-ylmethyl)-6,8- dihydro-2H,7H-spiro[furo[2,3- e]isoindole-3,4′-piperidin]-7- yl)piperidine-2,6-dione |
| A42 | 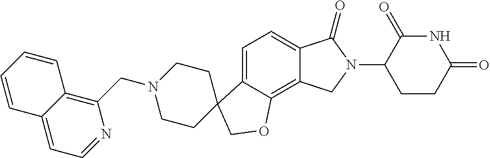 [SMILES: O=C1CCC(N2Cc3c(ccc4c3OCC43CCN(Cc4nccc5ccccc45)CC3)C2=O)C(=O)N1] | 3-(1′-(isoquinolin-1-ylmethyl)-6-oxo- 6,8-dihydro-2H,7H-spiro[furo[2,3- e]isoindole-3,4′-piperidin]-7- yl)piperidine-2,6-dione |
| A43 | 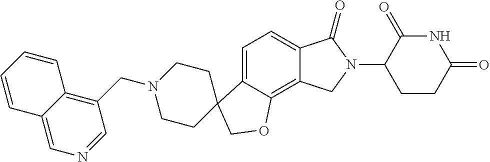 [SMILES: O=C1CCC(N2Cc3c(ccc4c3OCC43CCN(Cc4cncc5ccccc45)CC3)C2=O)C(=O)N1] | 3-(1′-(isoquinolin-4-ylmethyl)-6-oxo- 6,8-dihydro-2H,7H-spiro[furo[2,3- e]isoindole-3,4′-piperidin]-7- yl)piperidine-2,6-dione |
| A44 | 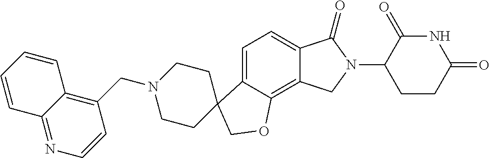 [SMILES: O=C1CCC(N2Cc3c(ccc4c3OCC43CCN(Cc4ccnc5ccccc45)CC3)C2=O)C(=O)N1] | 3-(6-oxo-1′-(quinolin-4-ylmethyl)-6,8- dihydro-2H,7H-spiro[furo[2,3- e]isoindole-3,4′-piperidin]-7- yl)piperidine-2,6-dione |
| A45 | 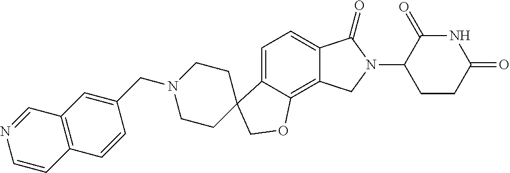 [SMILES: O=C1CCC(N2Cc3c(ccc4c3OCC43CCN(Cc4ccc5ccncc5c4)CC3)C2=O)C(=O)N1] | 3-(1′-(isoquinolin-7-ylmethyl)-6-oxo- 6,8-dihydro-2H,7H-spiro[furo[2,3- e]isoindole-3,4′-piperidin-7- yl)piperidine-2,6-dione |
| A46 | 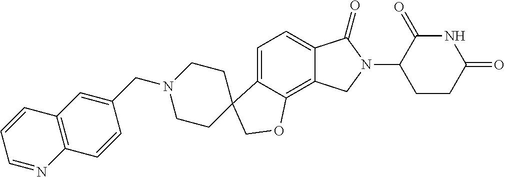 [SMILES: O=C1CCC(N2Cc3c(ccc4c3OCC43CCN(Cc4ccc5ncccc5c4)CC3)C2=O)C(=O)N1] | 3-(6-oxo-1′-(quinolin-6-ylmethyl)-6,8- dihydro-2H,7H-spiro[furo[2,3- e]isoindole-3,4′-piperidin]-7- yl)piperidine-2,6-dione |
| A47 | 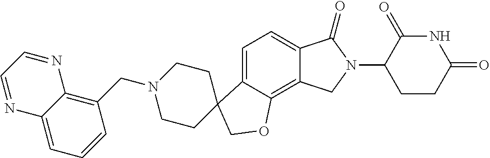 [SMILES: O=C1CCC(N2Cc3c(ccc4c3OCC43CCN(Cc4cccc5nccnc45)CC3)C2=O)C(=O)N1] | 3-(6-oxo-1′-(quinoxalin-5-ylmethyl)- 6,8-dihydro-2H,7H-spiro[furo[2,3- e]isoindole-3,4′-piperidin]-7- yl)piperidine-2,6-dione |
| A48 | 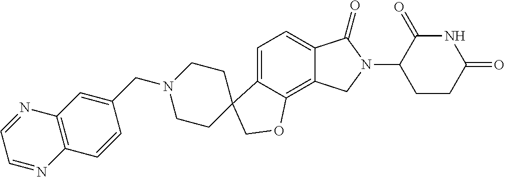 [SMILES: O=C1CCC(N2Cc3c(ccc4c3OCC43CCN(Cc4ccc5nccnc5c4)CC3)C2=O)C(=O)N1] | 3-(6-oxo-1′-(quinoxalin-6-ylmethyl)- 6,8-dihydro-2H,7H-spiro[furo[2,3- e]isoindole-3,4′-piperidin]-7- yl)piperidine-2,6-dione |
| A49 | 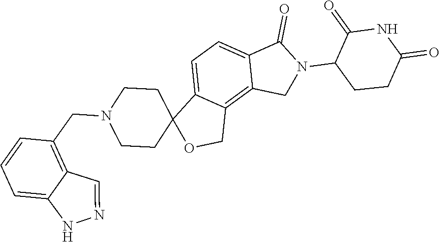 [SMILES: O=C1CCC(N2Cc3c(ccc4c3COC43CCN(Cc4cccc5[nH]ncc45)CC3)C2=O)C(=O)N1] | 3-(1′-((1H-indazol-4-yl)methyl)-6- oxo-6,8-dihydro-2H,7H- spiro[furo[2,3-e]isoindole-3,4′- piperidin]-7-yl)piperidine-2,6-dione |
| A50 | 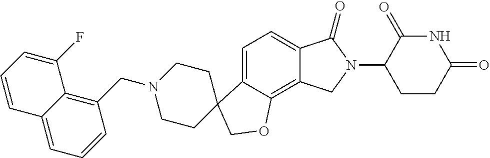 [SMILES: O=C1CCC(N2Cc3c(ccc4c3OCC43CCN(Cc4cccc5cccc(F)c45)CC3)C2=O)C(=O)N1] | 3-(1′-((8-fluoronaphthalen-1- yl)methyl)-6-oxo-6,8-dihydro-2H,7H- spiro[furo[2,3-e]isoindole-3,4′- piperidin]-7-yl)piperidine-2,6-dione |
| A51 | 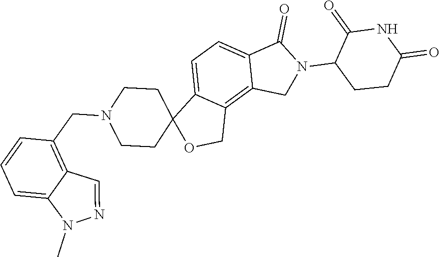 [SMILES: Cn1ncc2c(CN3CCC4(CC3)OCc3c4ccc4c3CN(C3CCC(=O)NC3=O)C4=O)cccc21] | 3-(1′-((1-methyl-1H-indazol-4- yl)methyl)-6-oxo-6,8-dihydro-2H,7H- spiro[furo[2,3-e]isoindole-3,4′- piperidin]-7-yl)piperidine-2,6-dione |
| A52 | 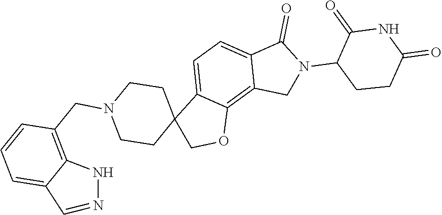 [SMILES: O=C1CCC(N2Cc3c(ccc4c3OCC43CCN(Cc4cccc5cn[nH]c45)CC3)C2=O)C(=O)N1] | 3-(1′-((1H-indazol-7-yl)methyl)-6- oxo-6,8-dihydro-2H,7H- spiro[furo[2,3-e]isoindole-3,4′- piperidin]-7-yl)piperidine-2,6-dione |
| A53 | 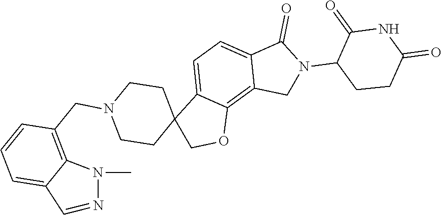 [SMILES: Cn1ncc2cccc(CN3CCC4(CC3)COc3c4ccc4c3CN(C3CCC(=O)NC3=O)C4=O)c21] | 3-(1′-((1-methyl-1H-indazol-7- yl)methyl)-6-oxo-6,8-dihydro-2H,7H- spiro[furo[2,3-e]isoindole-3,4′- piperidin]-7-yl)piperidine-2,6-dione |
| A54 | 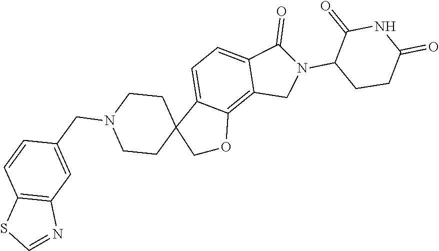 [SMILES: O=C1CCC(N2Cc3c(ccc4c3OCC43CCN(Cc4ccc5scnc5c4)CC3)C2=O)C(=O)N1] | 3-(1′-(benzo[d]thiazol-5-ylmethyl)-6- oxo-6,8-dihydro-2H,7H- spiro[furo[2,3-e]isoindole-3,4′- piperidin]-7-yl)piperidine-2,6-dione |
| A56 | 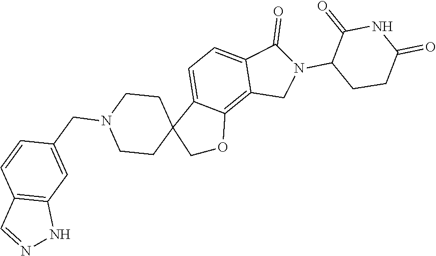 [SMILES: O=C1CCC(N2Cc3c(ccc4c3OCC43CCN(Cc4ccc5cn[nH]c5c4)CC3)C2=O)C(=O)N1] | 3-(1′-((1H-indazol-6-yl)methyl)-6- oxo-6,8-dihydro-2H,7H- spiro[furo[2,3-e]isoindole-3,4′- piperidin]-7-yl)piperidine-2,6-dione |
| A57 | 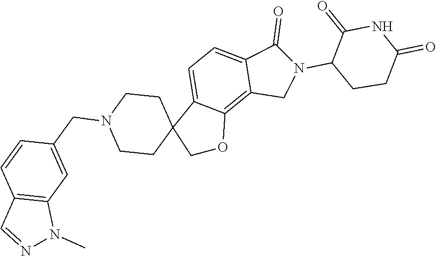 [SMILES: Cn1ncc2ccc(CN3CCC4(CC3)COc3c4ccc4c3CN(C3CCC(=O)NC3=O)C4=O)cc21] | 3-(1′-((1-methyl-1H-indazol-6- yl)methyl)-6-oxo-6,8-dihydro-2H,7H- spiro[furo[2,3-e]isoindole-3,4′- piperidin]-7-yl)piperidine-2,6-dione |
| A58 | 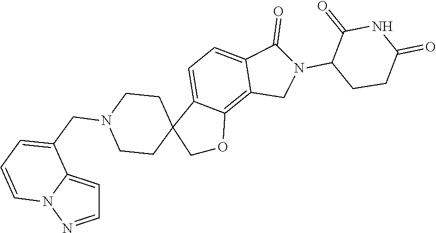 [SMILES: O=C1CCC(N2Cc3c(ccc4c3OCC43CCN(Cc4cccn5nccc45)CC3)C2=O)C(=O)N1] | 3-(6-oxo-1′-(pyrazolo[1,5-a]pyridin-4- ylmethyl)-6,8-dihydro-2H,7H- spiro[furo[2,3-e]isoindole-3,4′- piperidin]-7-yl)piperidine-2,6-dione |
| A59 | 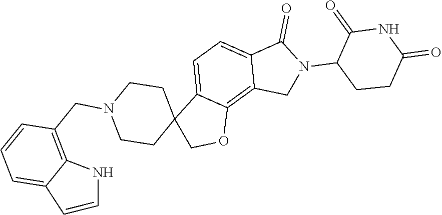 [SMILES: O=C1CCC(N2Cc3c(ccc4c3OCC43CCN(Cc4cccc5cc[nH]c45)CC3)C2=O)C(=O)N1] | 3-(1′-((1H-indol-7-yl)methyl)-6-oxo- 6,8-dihydro-2H,7H-spiro[furo[2,3- e]isoindole-3,4′-piperidin]-7- yl)piperidine-2,6-dione |
| A60 | 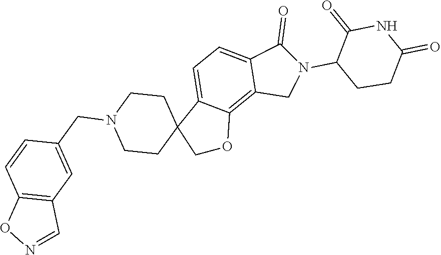 [SMILES: O=C1CCC(N2Cc3c(ccc4c3OCC43CCN(Cc4ccc5oncc5c4)CC3)C2=O)C(=O)N1] | 3-(1′-(benzo[d]isoxazol-5-ylmethyl)- 6-oxo-6,8-dihydro-2H,7H- spiro[furo[2,3-e]isoindole-3,4′- piperidin]-7-yl)piperidine-2,6-dione |
| A62 | 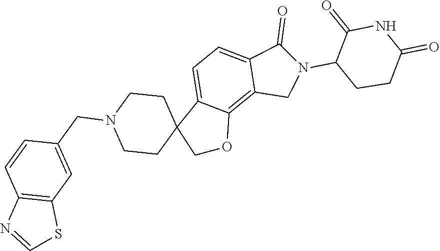 [SMILES: O=C1CCC(N2Cc3c(ccc4c3OCC43CCN(Cc4ccc5ncsc5c4)CC3)C2=O)C(=O)N1] | 3-(1′-(benzo[d]thiazol-6-ylmethyl)-6- oxo-6,8-dihydro-2H,7H- spiro[furo[2,3-e]isoindole-3,4′- piperidin]-7-yl)piperidine-2,6-dione |
| A64 | 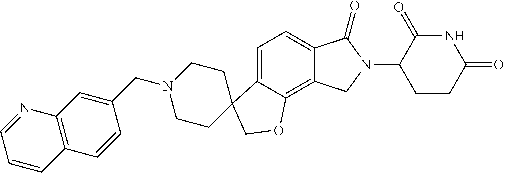 [SMILES: O=C1CCC(N2Cc3c(ccc4c3OCC43CCN(Cc4ccc5cccnc5c4)CC3)C2=O)C(=O)N1] | 3-(6-oxo-1′-(quinolin-7-ylmethyl)-6,8- dihydro-2H,7H-spiro[furo[2,3- e]isoindole-3,4′-piperidin]-7- yl)piperidine-2,6-dione |
| A65 | 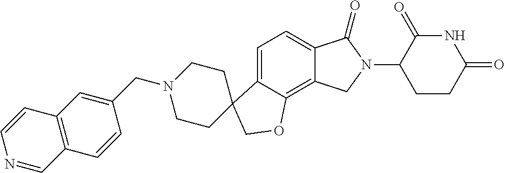 [SMILES: O=C1CCC(N2Cc3c(ccc4c3OCC43CCN(Cc4ccc5cnccc5c4)CC3)C2=O)C(=O)N1] | 3-(1′-(isoquinolin-6-ylmethyl)-6-oxo- 6,8-dihydro-2H,7H-spiro[furo[2,3- e]isoindole-3,4′-piperidin]-7- yl)piperidine-2,6-dione |
| A66 | 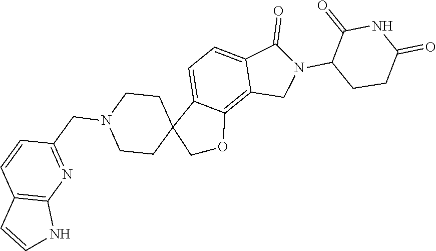 [SMILES: O=C1CCC(N2Cc3c(ccc4c3OCC43CCN(Cc4ccc5cc[nH]c5n4)CC3)C2=O)C(=O)N1] | 3-(1′-((1H-pyrrolo[2,3-b]pyridin-6- yl)methyl)-6-oxo-6,8-dihydro-2H,7H- spiro[furo[2,3-e]isoindole-3,4′- piperidin]-7-yl)piperidine-2,6-dione |
| A67 | 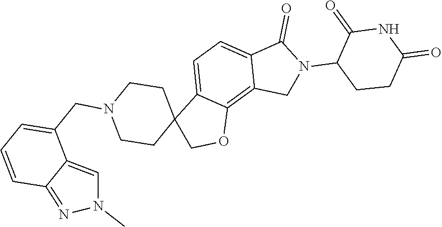 [SMILES: Cn1cc2c(CN3CCC4(CC3)COc3c4ccc4c3CN(C3CCC(=O)NC3=O)C4=O)cccc2n1] | 3-(1′-((2-methyl-2H-indazol-4- yl)methyl)-6-oxo-6,8-dihydro-2H,7H- spiro[furo[2,3-e]isoindole-3,4′- piperidin]-7-yl)piperidine-2,6-dione |
| A68 | 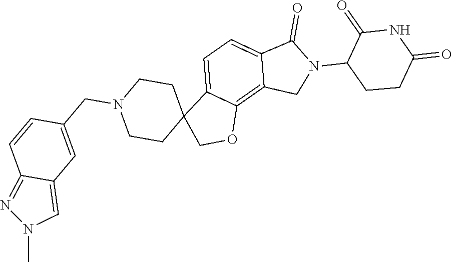 [SMILES: Cn1cc2cc(CN3CCC4(CC3)COc3c4ccc4c3CN(C3CCC(=O)NC3=O)C4=O)ccc2n1] | 3-(1′-((2-methyl-2H-indazol-5- yl)methyl)-6-oxo-6,8-dihydro-2H,7H- spiro[furo[2,3-e]isoindole-3,4′- piperidin]-7-yl)piperidine-2,6-dione |
| A69 | 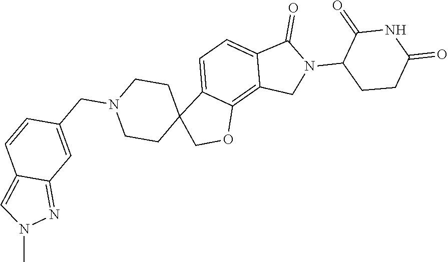 [SMILES: Cn1cc2ccc(CN3CCC4(CC3)COc3c4ccc4c3CN(C3CCC(=O)NC3=O)C4=O)cc2n1] | 3-(1′-((2-methyl-2H-indazol-6- yl)methyl)-6-oxo-6,8-dihydro-2H,7H- spiro[furo[2,3-e]isoindole-3,4′- piperidin]-7-yl)piperidine-2,6-dione |
| A70 | 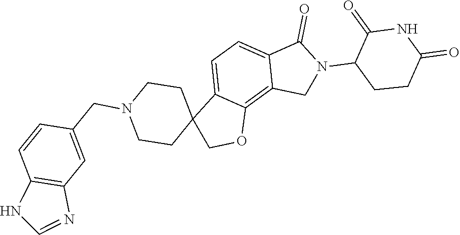 [SMILES: O=C1CCC(N2Cc3c(ccc4c3OCC43CCN(Cc4ccc5[nH]cnc5c4)CC3)C2=O)C(=O)N1] | 3-(1′-((1H-benzo[d]imidazol-5- yl)methyl)-6-oxo-6,8-dihydro-2H,7H- spiro[furo[2,3-e]isoindole-3,4′- piperidin]-7-yl)piperidine-2,6-dione |
| A71 | 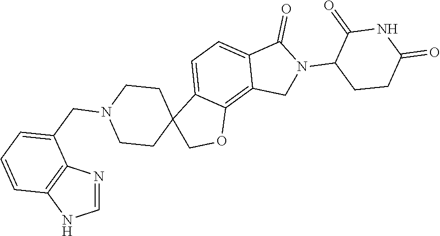 [SMILES: O=C1CCC(N2Cc3c(ccc4c3OCC43CCN(Cc4cccc5[nH]cnc45)CC3)C2=O)C(=O)N1] | 3-(1′-((1H-benzo[d]imidazol-4- yl)methyl)-6-oxo-6,8-dihydro-2H,7H- spiro[furo[2,3-e]isoindole-3,4′- piperidin]-7-yl)piperidine-2,6-dione |
| A72 | 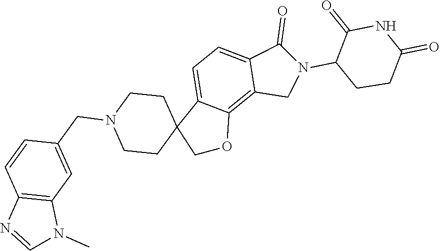 [SMILES: Cn1cnc2ccc(CN3CCC4(CC3)COc3c4ccc4c3CN(C3CCC(=O)NC3=O)C4=O)cc21] | 3-(1′-((1-methyl-1H- benzo[d]imidazol-6-yl)methyl)-6-oxo- 6,8-dihydro-2H,7H-spiro[furo[2,3- e]isoindole-3,4′-piperidin]-7- yl)piperidine-2,6-dione |
| A73 | 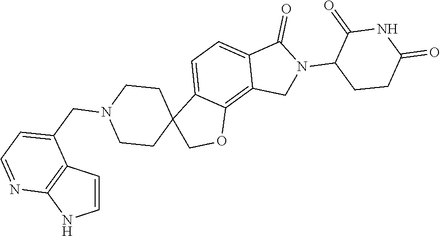 [SMILES: O=C1CCC(N2Cc3c(ccc4c3OCC43CCN(Cc4ccnc5[nH]ccc45)CC3)C2=O)C(=O)N1] | 3-(1′-((1H-pyrrolo[2,3-b]pyridin-4- yl)methyl)-6-oxo-6,8-dihydro-2H,7H- spiro[furo[2,3-e]isoindole-3,4′- piperidin]-7-yl)piperidine-2,6-dione |
| A74 | 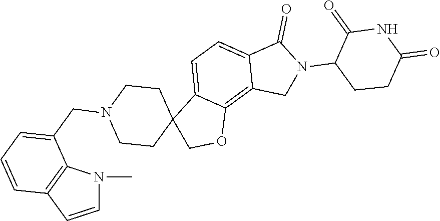 [SMILES: Cn1ccc2cccc(CN3CCC4(CC3)COc3c4ccc4c3CN(C3CCC(=O)NC3=O)C4=O)c21] | 3-(1′-((1-methyl-1H-indol-7- yl)methyl)-6-oxo-6,8-dihydro-2H,7H- spiro[furo[2,3-e]isoindole-3,4′- piperidin]-7-yl)piperidine-2,6-dione |
| A75 | 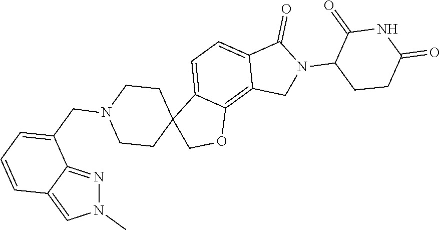 [SMILES: Cn1cc2cccc(CN3CCC4(CC3)COc3c4ccc4c3CN(C3CCC(=O)NC3=O)C4=O)c2n1] | 3-(1′-((2-methyl-2H-indazol-7- yl)methyl)-6-oxo-6,8-dihydro-2H,7H- spiro[furo[2,3-e]isoindole-3,4′- piperidin]-7- yl)piperidine-2,6-dione |
| A76 | 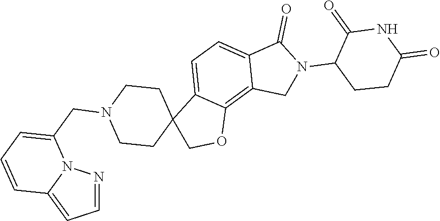 [SMILES: O=C1CCC(N2Cc3c(ccc4c3OCC43CCN(Cc4cccc5ccnn45)CC3)C2=O)C(=O)N1] | 3-(6-oxo-1′-(pyrazolo[1,5-]pyridin-7- ylmethyl)-6,8-dihydro-2H,7H- spiro[furo[2,3-e]isoindole-3,4′- piperidin]-7-yl)piperidine-2,6-dione |
| A77 | 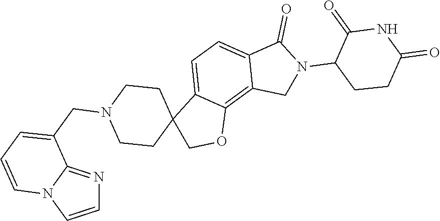 [SMILES: O=C1CCC(N2Cc3c(ccc4c3OCC43CCN(Cc4cccn5ccnc45)CC3)C2=O)C(=O)N1] | 3-(1′-(imidazo[1,2-a]pyridin-8- ylmethyl)-6-oxo-6,8-dihydro-2H,7H- spiro[furo[2,3-e]isoindole-3,4′- piperidin]-7-yl)piperidine-2,6-dione |
| A78 | 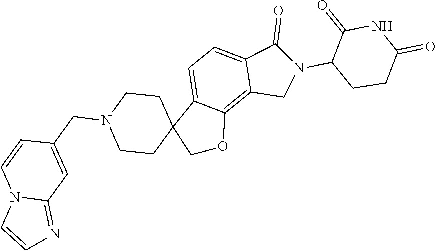 [SMILES: O=C1CCC(N2Cc3c(ccc4c3OCC43CCN(Cc4ccn5ccnc5c4)CC3)C2=O)C(=O)N1] | 3-(1′-(imidazo[1,2-a]pyridin-7- yl)methyl)-6-oxo-6,8-dihydro-2H,7H- spiro[furo[2,3-e]isoindole-3,4′- piperidin]-7-yl)piperidine-2,6-dione |
| A79 | 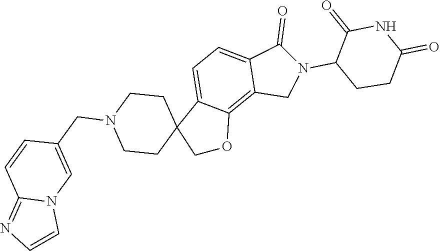 [SMILES: O=C1CCC(N2Cc3c(ccc4c3OCC43CCN(Cc4ccc5nccn5c4)CC3)C2=O)C(=O)N1] | 3-(1′-(imidazo[1,2-a]pyridin-6- yl)methyl)-6-oxo-6,8-dihydro-2H,7H- spiro[furo[2,3-e]isoindole-3,4′- piperidin]-7-yl)piperidine-2,6-dione |
| A80 | 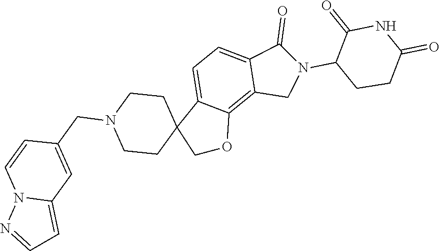 [SMILES: O=C1CCC(N2Cc3c(ccc4c3OCC43CCN(Cc4ccn5nccc5c4)CC3)C2=O)C(=O)N1] | 3-(6-oxo-1′-(pyrazolo[1,5-a]pyridin-5- ylmethyl)-6,8-dihydro-2H,7H- spiro[furo[2,3-e]isoindole-3,4′- piperidin]-7-yl)piperidine-2,6-dione |
| A81 | 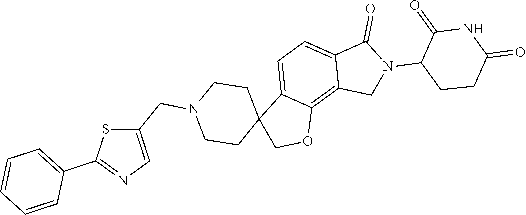 [SMILES: O=C1CCC(N2Cc3c(ccc4c3OCC43CCN(Cc4cnc(-c5ccccc5)s4)CC3)C2=O)C(=O)N1] | 3-(6-oxo-1′-((2-phenylthiazol-5- yl)methyl)-6,8-dihydro-2H,7H- spiro[furo[2,3-e]isoindole-3,4′- piperidin]-7-yl)piperidine-2,6-dione |
| A82 | 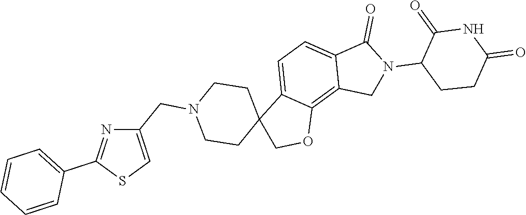 [SMILES: O=C1CCC(N2Cc3c(ccc4c3OCC43CCN(Cc4csc(-c5ccccc5)n4)CC3)C2=O)C(=O)N1] | 3-(6-oxo-1′-((2-phenylthiazol-4- yl)methyl)-6,8-dihydro-2H,7H- spiro[furo[2,3-e]isoindole-3,4′- piperidin]-7-yl)piperidine-2,6-dione |
| A83 | 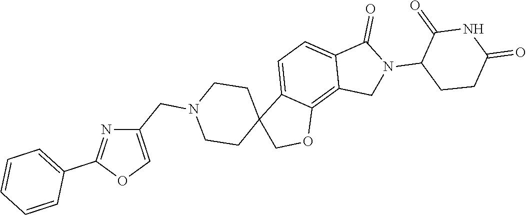 [SMILES: O=C1CCC(N2Cc3c(ccc4c3OCC43CCN(Cc4coc(-c5ccccc5)n4)CC3)C2=O)C(=O)N1] | 3-(6-oxo-1′-((2-phenyloxazol-4- yl)methyl)-6,8-dihydro-2H,7H- spiro[furo[2,3-e]isoindole-3,4′- piperidin]-7-yl)piperidine-2,6-dione |
| A84 | 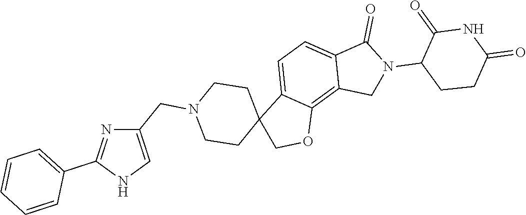 [SMILES: O=C1CCC(N2Cc3c(ccc4c3OCC43CCN(Cc4c[nH]c(-c5ccccc5)n4)CC3)C2=O)C(=O)N1] | 3-(6-oxo-1′-((2-phenyl-1H-imidazol- 4-yl)methyl)-6,8-dihydro-2H,7H- spiro[furo[2,3-e]isoindole-3,4′- piperidin]-7-yl)piperidine-2,6-dione |
| A85 | 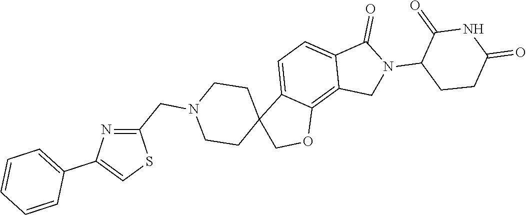 [SMILES: O=C1CCC(N2Cc3c(ccc4c3OCC43CCN(Cc4nc(-c5ccccc5)cs4)CC3)C2=O)C(=O)N1] | 3-(6-oxo-1′-((4-phenylthiazol-2- yl)methyl)-6,8-dihydro-2H,7H- spiro[furo[2,3-e]isoindole-3,4′- piperidin]-7-yl)piperidine-2,6-dione |
| A86 | 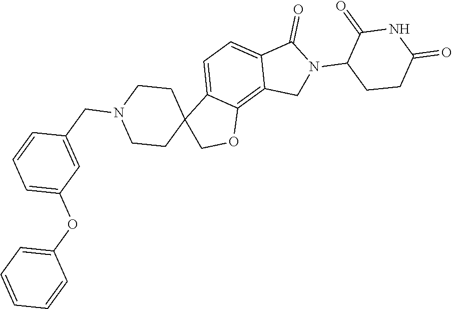 [SMILES: O=C1CCC(N2Cc3c(ccc4c3OCC43CCN(Cc4cccc(Oc5ccccc5)c4)CC3)C2=O)C(=O)N1] | 3-(6-oxo-1′-(3-phenoxybenzyl)-6,8- dihydro-2H,7H- spiro[furo[2,3-e]isoindole-3,4′- piperidin]-7-yl)piperidine-2,6-dione |
| A87 | 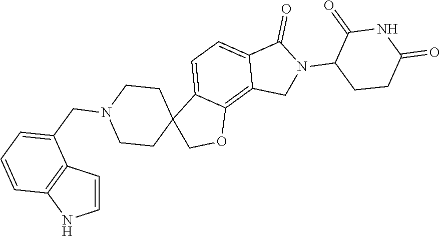 [SMILES: O=C1CCC(N2Cc3c(ccc4c3OCC43CCN(Cc4cccc5[nH]ccc45)CC3)C2=O)C(=O)N1] | 3-(1′-((1H-indol-4-yl)methyl)-6-oxo- 6,8-dihydro-2H,7H- spiro[furo[2,3-e]isoindole-3,4′- piperidin]-7-yl)piperidine-2,6-dione |
| A88 | 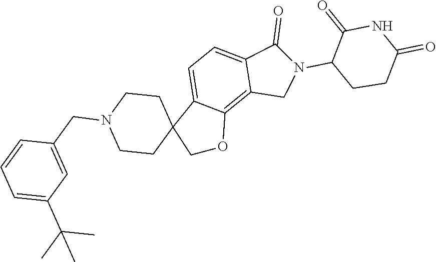 [SMILES: CC(C)(C)c1cccc(CN2CCC3(CC2)COc2c3ccc3c2CN(C2CCC(=O)NC2=O)C3=O)c1] | 3-(1′-(3-(tert-butyl)benzyl)-6-oxo-6,8- dihydro-2H,7H- spiro[furo[2,3-e]isoindole-3,4′- piperidin]-7-yl)piperidine-2,6-dione |
| A89 | 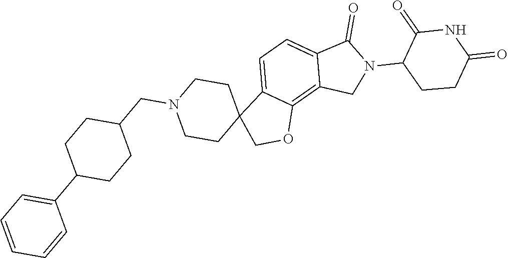 [SMILES: O=C1CCC(N2Cc3c(ccc4c3OCC43CCN(CC4CCC(c5ccccc5)CC4)CC3)C2=O)C(=O)N1] | 3-(6-oxo-1′-((4- phenylcyclohexyl)methyl)-6,8- dihydro-2H,7H- spiro[furo[2,3-e]isoindole-3,4′- piperidin]-7-yl)piperidine-2,6-dione |
| A90 | 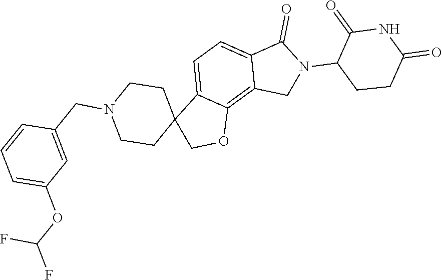 [SMILES: O=C1CCC(N2Cc3c(ccc4c3OCC43CCN(Cc4cccc(OC(F)F)c4)CC3)C2=O)C(=O)N1] | 3-(1′-(3-(difluoromethoxy)benzyl)-6- oxo-6,8-dihydro-2H,7H- spiro[furo[2,3-e]isoindole-3,4′- piperidin]-7-yl)piperidine-2,6-dione |
| A91 | 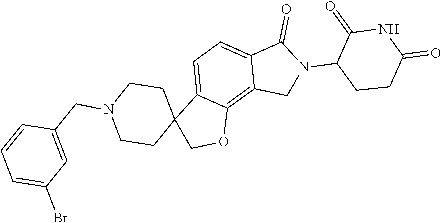 [SMILES: O=C1CCC(N2Cc3c(ccc4c3OCC43CCN(Cc4cccc(Br)c4)CC3)C2=O)C(=O)N1] | 3-(1′-(3-bromobenzyl)-6-oxo-6,8- dihydro-2H,7H- spiro[furo[2,3-e]isoindole-3,4′- piperidin]-7-yl)piperidine-2,6-dione |
| A92 | 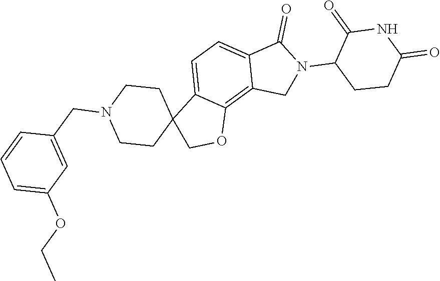 [SMILES: CCOc1cccc(CN2CCC3(CC2)COc2c3ccc3c2CN(C2CCC(=O)NC2=O)C3=O)c1] | 3-(1′-(3-ethoxybenzyl)-6-oxo-6,8- dihydro-2H,7H- spiro[furo[2,3-e]isoindole-3,4′- piperidin]-7-yl)piperidine-2,6-dione |
| A93 | 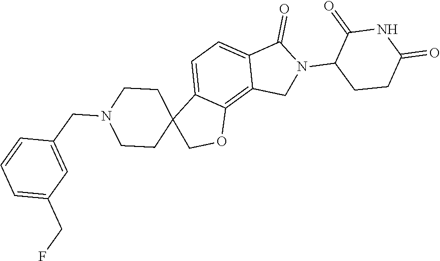 [SMILES: O=C1CCC(N2Cc3c(ccc4c3OCC43CCN(Cc4cccc(CF)c4)CC3)C2=O)C(=O)N1] | 3-(1′-(3-(fluoromethyl)benzyl)-6-oxo- 6,8-dihydro-2H,7H- spiro[furo[2,3-e]isoindole-3,4′- piperidin]-7-yl)piperidine-2,6-dione |
| A94 | 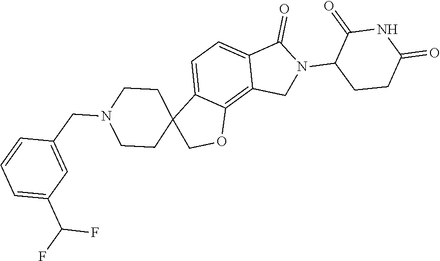 [SMILES: O=C1CCC(N2Cc3c(ccc4c3OCC43CCN(Cc4cccc(C(F)F)c4)CC3)C2=O)C(=O)N1] | 3-(1′-(3-(difluoromethyl)benzyl)-6- oxo-6,8-dihydro-2H,7H- spiro[furo[2,3-e]isoindole-3,4′- piperidin]-7-yl)piperidine-2,6-dione |
| A95 | 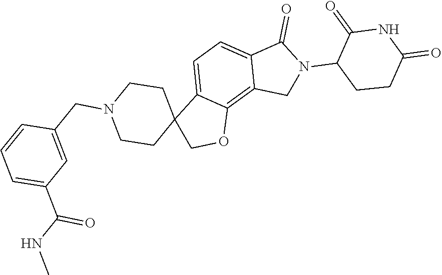 [SMILES: CNC(=O)c1cccc(CN2CCC3(CC2)COc2c3ccc3c2CN(C2CCC(=O)NC2=O)C3=O)c1] | 3-((7-(2,6-dioxopiperidin-3-yl)-6-oxo- 7,8-dihydro-2H,6H- spiro[furo[2,3-e]isoindole-3,4′- piperidin]-1′-yl)methyl-N- methylbenzamide |
| A96 | 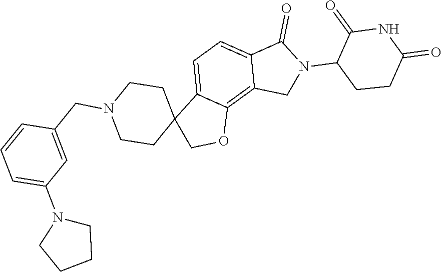 [SMILES: O=C1CCC(N2Cc3c(ccc4c3OCC43CCN(Cc4cccc(N5CCCC5)c4)CC3)C2=O)C(=O)N1] | 3-(6-oxo-1′-(3-(pyrrolidin-1- yl)benzyl)-6,8-dihydro-2H,7H- spiro[furo[2,3-e]isoindole-3,4′- piperidin]-7-yl)piperidine-2,6-dione |
| A97 | 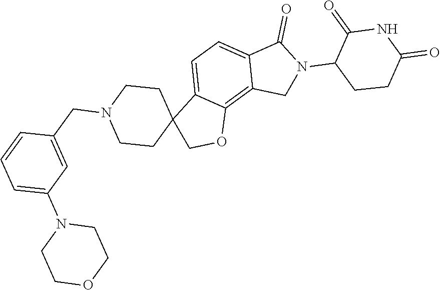 [SMILES: O=C1CCC(N2Cc3c(ccc4c3OCC43CCN(Cc4cccc(N5CCOCC5)c4)CC3)C2=O)C(=O)N1] | 3-(1′-(3-morpholinobenzyl)-6-oxo-6,8- dihydro-2H,7H- spiro[furo[2,3-e]isoindole-3,4′- piperidin]-7-yl)piperidine-2,6-dione |
| A98 | 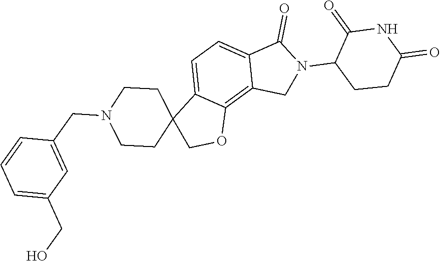 [SMILES: O=C1CCC(N2Cc3c(ccc4c3OCC43CCN(Cc4cccc(CO)c4)CC3)C2=O)C(=O)N1] | 3-(1′-(3-(hydroxymethyl)benzyl)-6- oxo-6,8-dihydro-2H,7H- spiro[furo[2,3-e]isoindole-3,4′- piperidin]-7-yl)piperidine-2,6-dione |
| A99 | 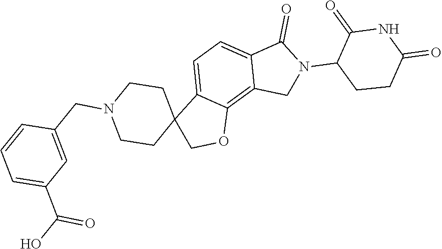 [SMILES: O=C1CCC(N2Cc3c(ccc4c3OCC43CCN(Cc4cccc(C(=O)O)c4)CC3)C2=O)C(=O)N1] | 3-((7-(2,6-dioxopiperidin-3-yl)-6-oxo- 7,8-dihydro-2H,6H- spiro[furo[2,3-e]isoindole-3,4′- piperidin]-1′-yl)methyl)benzoic acid |
| A100 | 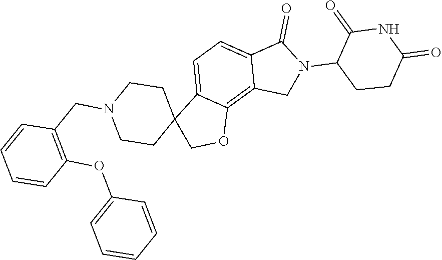 [SMILES: O=C1CCC(N2Cc3c(ccc4c3OCC43CCN(Cc4ccccc4Oc4ccccc4)CC3)C2=O)C(=O)N1] | 3-(6-oxo-1′-(2-phenoxybenzyl)-6,8- dihydro-2H,7H- spiro[furo[2,3-e]isoindole-3,4′- piperidin]-7-yl)piperidine-2,6-dione |
| A101 | 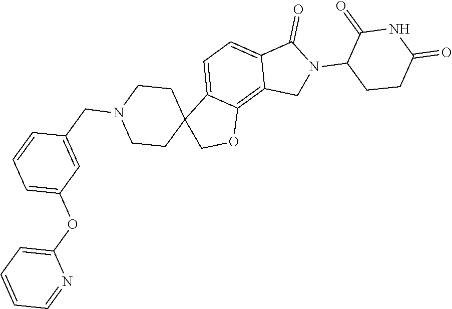 [SMILES: O=C1CCC(N2Cc3c(ccc4c3OCC43CCN(Cc4cccc(Oc5ccccn5)c4)CC3)C2=O)C(=O)N1] | 3-(6-oxo-1′-(3-(pyridin-2- yloxy)benzyl)-6,8-dihydro-2H,7H- spiro[furo[2,3-e]isoindole-3,4′- piperidin]-7-yl)piperidine-2,6-dione |
| A102 | 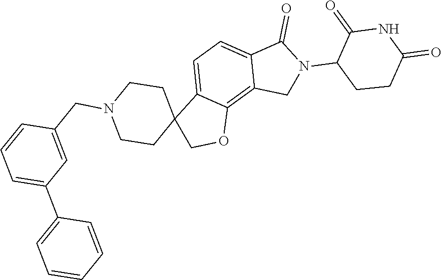 [SMILES: O=C1CCC(N2Cc3c(ccc4c3OCC43CCN(Cc4cccc(-c5ccccc5)c4)CC3)C2=O)C(=O)N1] | 3-(1′-([1,1′-biphenyl]-3-ylmethyl)-6- oxo-6,8-dihydro-2H,7H- spiro[furo[2,3-e]isoindole-3,4′- piperidin]-7-yl)piperidine-2,6-dione |
| A103 | 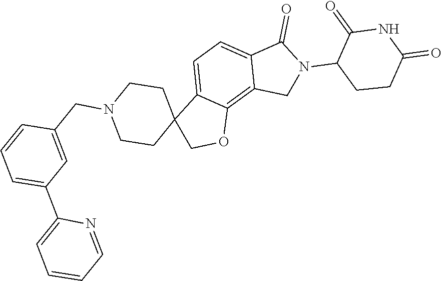 [SMILES: O=C1CCC(N2Cc3c(ccc4c3OCC43CCN(Cc4cccc(-c5ccccn5)c4)CC3)C2=O)C(=O)N1] | 3-(6-oxo-1′-(3-(pyridin-2-yl)benzyl)- 6,8-dihydro-2H,7H- spiro[furo[2,3-e]isoindole-3,4′- piperidin]-7-yl)piperidine-2,6-dione |
| A104 | 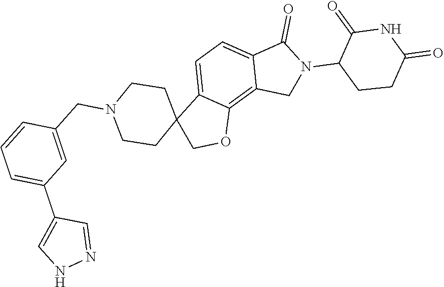 [SMILES: O=C1CCC(N2Cc3c(ccc4c3OCC43CCN(Cc4cccc(-c5cn[nH]c5)c4)CC3)C2=O)C(=O)N1] | 3-(1′-(3-(1H-pyrazol-4-yl)benzyl)-6- oxo-6,8-dihydro-2H,7H- spiro[furo[2,3-e]isoindole-3,4′- piperidin]-7-yl)piperidine-2,6-dione |
| A105 | 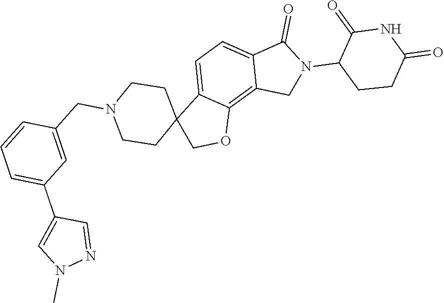 [SMILES: Cn1cc(-c2cccc(CN3CCC4(CC3)COc3c4ccc4c3CN(C3CCC(=O)NC3=O)C4=O)c2)cn1] | 3-(1′-(3-(1-methyl-1H-pyrazol-4- yl)benzyl)-6-oxo-6,8-dihydro-2H,7H- spiro[furo[2,3-e]isoindole-3,4′- piperidin]-7-yl)piperidine-2,6-dione |
| A106 | 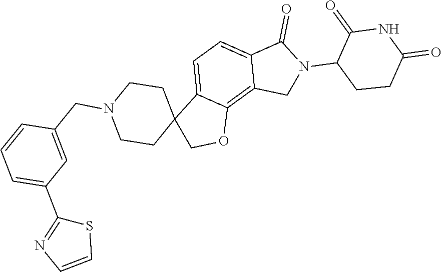 [SMILES: O=C1CCC(N2Cc3c(ccc4c3OCC43CCN(Cc4cccc(-c5nccs5)c4)CC3)C2=O)C(=O)N1] | 3-(6-oxo-1′-(3-(thiazol-2-yl)benzyl)- 6,8-dihydro-2H,7H- spiro[furo[2,3-e]isoindole-3,4′- piperidin]-7-yl)piperidine-2,6-dione |
| A107 | 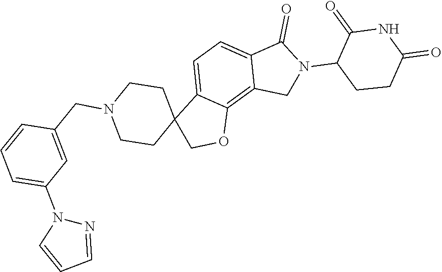 [SMILES: O=C1CCC(N2Cc3c(ccc4c3OCC43CCN(Cc4cccc(-n5cccn5)c4)CC3)C2=O)C(=O)N1] | 3-(1′-(3-(1H-pyrazol-1-yl)benzyl)-6- oxo-6,8-dihydro-2H,7H- spiro[furo[2,3-e]isoindole-3,4′- piperidin]-7-yl)piperidine-2,6-dione |
| A108 | 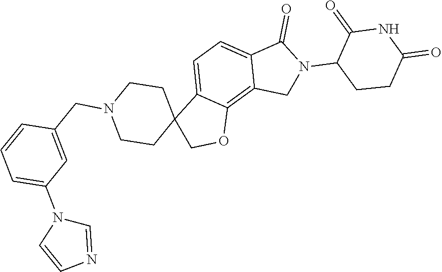 [SMILES: O=C1CCC(N2Cc3c(ccc4c3OCC43CCN(Cc4cccc(-n5ccnc5)c4)CC3)C2=O)C(=O)N1] | 3-(1′-(3-(1H-imidazol-1-yl)benzyl)-6- oxo-6,8-dihydro-2H,7H- spiro[furo[2,3-e]isoindole-3,4′- piperidin]-7-yl)piperidine-2,6-dione |
| A109 | 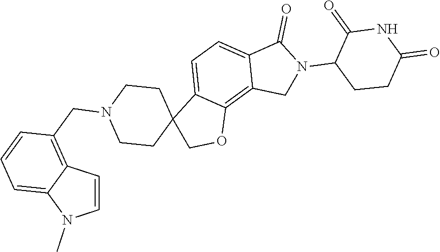 [SMILES: Cn1ccc2c(CN3CCC4(CC3)COc3c4ccc4c3CN(C3CCC(=O)NC3=O)C4=O)cccc21] | 3-(1′-((1-methyl-1H-indol-4- yl)methyl)-6-oxo-6,8-dihydro-2H,7H- spiro[furo[2,3-e]isoindole-3,4′- piperidin]-7-yl)piperidine-2,6-dione |
| A110 | 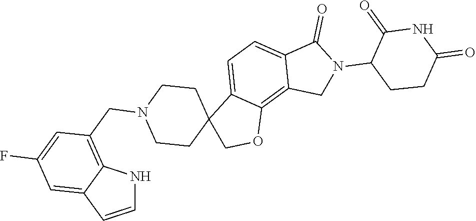 [SMILES: O=C1CCC(N2Cc3c(ccc4c3OCC43CCN(Cc4cc(F)cc5cc[nH]c45)CC3)C2=O)C(=O)N1] | 3-(1′-((5-fluoro-1H-indol-7- yl)methyl)-6-oxo-6,8-dihydro-2H,7H- spiro[furo[2,3-e]isoindole-3,4′- piperidin]-7-yl)piperidine-2,6-dione |
| A111 | 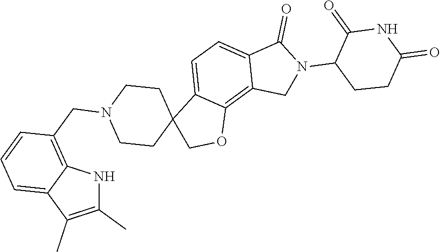 [SMILES: Cc1[nH]c2c(CN3CCC4(CC3)COc3c4ccc4c3CN(C3CCC(=O)NC3=O)C4=O)cccc2c1C] | 3-(1′-((2,3-dimethyl-1H-indol-7- yl)methyl)-6-oxo-6,8-dihydro-2H,7H- spiro[furo[2,3-e]isoindole-3,4′- piperidin]-7-yl)piperidine-2,6-dione |
| A112 | 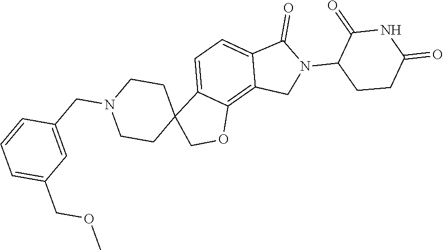 [SMILES: COCc1cccc(CN2CCC3(CC2)COc2c3ccc3c2CN(C2CCC(=O)NC2=O)C3=O)c1] | 3-(1′-(3-(methoxymethyl)benzyl)-6- oxo-6,8-dihydro-2H,7H- spiro[furo[2,3-e]isoindole-3,4′- piperidin]-7-yl)piperidine-2,6-dione |
| A113 | 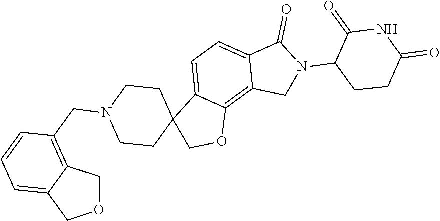 [SMILES: O=C1CCC(N2Cc3c(ccc4c3OCC43CCN(Cc4cccc5c4COC5)CC3)C2=O)C(=O)N1] | 3-(1′-((1,3-dihydroisobenzofuran-4- yl)methyl)-6-oxo-6,8-dihydro-2H,7H- spiro[furo[2,3-e]isoindole-3,4′- piperidin]-7-yl)piperidine-2,6-dione |
| A114 | 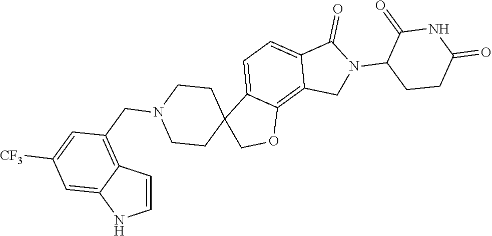 [SMILES: O=C1CCC(N2Cc3c(ccc4c3OCC43CCN(Cc4cc(C(F)(F)F)cc5[nH]ccc45)CC3)C2=O)C(=O)N1] | 3-(6-oxo-1′-((6-(trifluoromethyl)-1H- indol-4-yl)methyl)-6,8-dihydro-2H,7H- spiro[furo[2,3-e]isoindole-3,4′- piperidin]-7-yl)piperidine-2,6-dione |
| A115 | 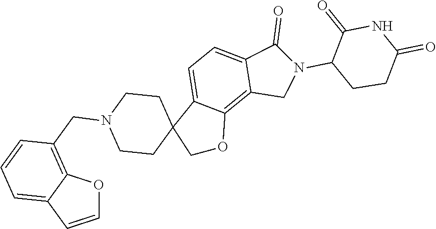 [SMILES: O=C1CCC(N2Cc3c(ccc4c3OCC43CCN(Cc4cccc5ccoc45)CC3)C2=O)C(=O)N1] | 3-(1′-(benzofuran-7-ylmethyl)-6-oxo- 6,8-dihydro-2H,7H- spiro[furo[2,3-e]isoindole-3,4′- piperidin]-7-yl)piperidine-2,6-dione |
| A116 | 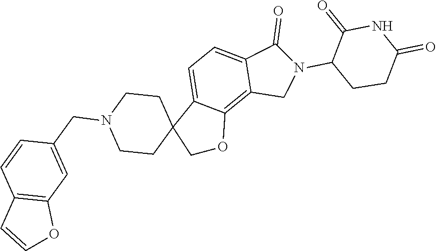 [SMILES: O=C1CCC(N2Cc3c(ccc4c3OCC43CCN(Cc4ccc5ccoc5c4)CC3)C2=O)C(=O)N1] | 3-(1′-(benzofuran-6-ylmethyl)-6-oxo- 6,8-dihydro-2H,7H- spiro[furo[2,3-e]isoindole-3,4′- piperidin]-7-yl)piperidine-2,6-dione |
| A117 | 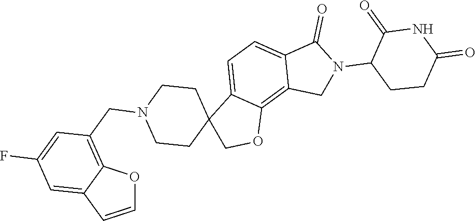 [SMILES: O=C1CCC(N2Cc3c(ccc4c3OCC43CCN(Cc4cc(F)cc5ccoc45)CC3)C2=O)C(=O)N1] | 3-(1′-((5-fluorobenzofuran-7- yl)methyl)-6-oxo-6,8-dihydro-2H,7H- spiro[furo[2,3-e]isoindole-3,4′- piperidin]-7-yl)piperidine-2,6-dione |
| A118 | 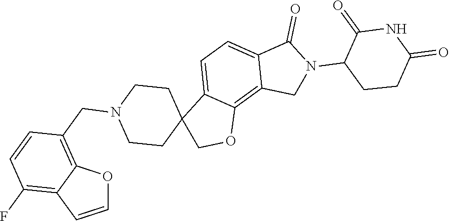 [SMILES: O=C1CCC(N2Cc3c(ccc4c3OCC43CCN(Cc4ccc(F)c5ccoc45)CC3)C2=O)C(=O)N1] | 3-(1′-((4-fluorobenzofuran-7- yl)methyl)-6-oxo-6,8-dihydro-2H,7H- spiro[furo[2,3-e]isoindole-3,4′- piperidin]-7-yl)piperidine-2,6-dione |
| A119 | 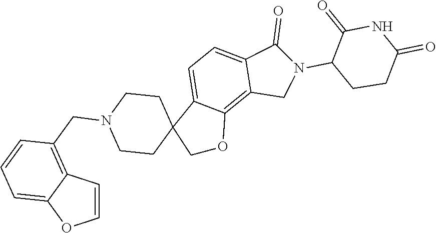 [SMILES: O=C1CCC(N2Cc3c(ccc4c3OCC43CCN(Cc4cccc5occc45)CC3)C2=O)C(=O)N1] | 3-(1′-(benzofuran-4-ylmethyl)-6-oxo- 6,8-dihydro-2H,7H- spiro[furo[2,3-e]isoindole-3,4′- piperidin]-7-yl)piperidine-2,6-dione |
| A120 | 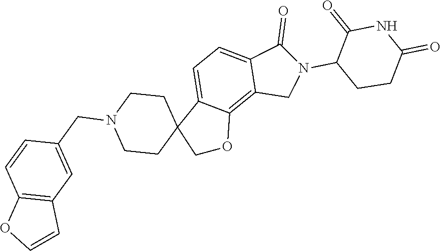 [SMILES: O=C1CCC(N2Cc3c(ccc4c3OCC43CCN(Cc4ccc5occc5c4)CC3)C2=O)C(=O)N1] | 3-(1′-(benzofuran-5-ylmethyl)-6-oxo- 6,8-dihydro-2H,7H- spiro[furo[2,3-e]isoindole-3,4′- piperidin]-7-yl)piperidine-2,6-dione |
| A121 | 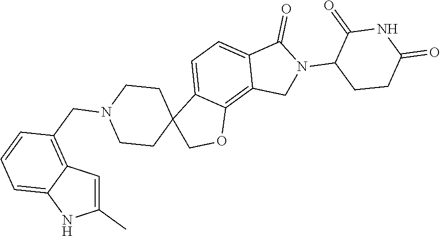 [SMILES: Cc1cc2c(CN3CCC4(CC3)COc3c4ccc4c3CN(C3CCC(=O)NC3=O)C4=O)cccc2[nH]1] | 3-(1′-((2-methyl-1H-indol-4- yl)methyl)-6-oxo-6,8-dihydro-2H,7H- spiro[furo[2,3-e]isoindole-3,4′- piperidin]-7-yl)piperidine-2,6-dione |
| A122 | 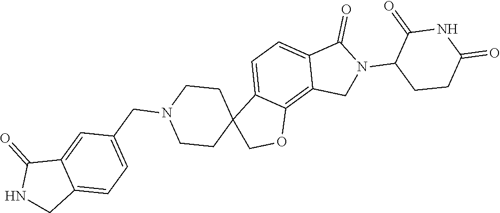 [SMILES: O=C1CCC(N2Cc3c(ccc4c3OCC43CCN(Cc4ccc5c(c4)C(=O)NC5)CC3)C2=O)C(=O)N1] | 3-(6-oxo-1′-((3-oxoisoindolin-5- yl)methyl)-6,8-dihydro-2H,7H- spiro[furo[2,3-e]isoindole-3,4′- piperidin]-7-yl)piperidine-2,6-dione |
| A123 | 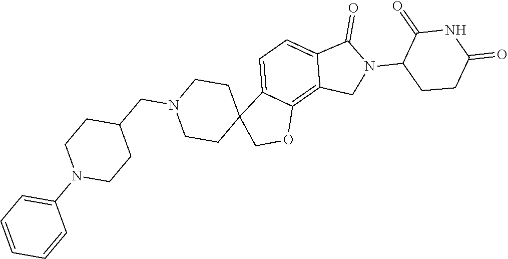 [SMILES: O=C1CCC(N2Cc3c(ccc4c3OCC43CCN(CC4CCN(c5ccccc5)CC4)CC3)C2=O)C(=O)N1] | 3-(6-oxo-1′-((1-phenylpiperidin-4- yl)methyl)-6,8-dihydro-2H,7H- spiro[furo[2,3-e]isoindole-3,4′- piperidin]-7-yl)piperidine-2,6-dione |
| A124 | 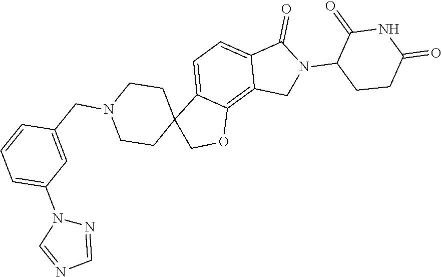 [SMILES: O=C1CCC(N2Cc3c(ccc4c3OCC43CCN(Cc4cccc(-n5cncn5)c4)CC3)C2=O)C(=O)N1] | 3-(1′-(3-(1H-1,2,4-triazol-1- yl)benzyl)-6-oxo-6,8-dihydro-2H,7H- spiro[furo[2,3-e]isoindole-3,4′- piperidin]-7-yl)piperidine-2,6-dione |
| A125 | 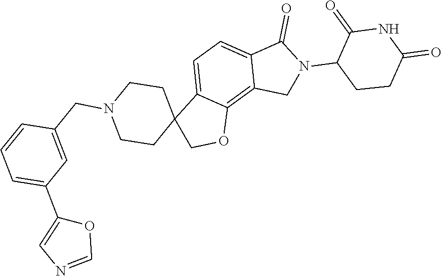 [SMILES: O=C1CCC(N2Cc3c(ccc4c3OCC43CCN(Cc4cccc(-c5cnco5)c4)CC3)C2=O)C(=O)N1] | 3-(1′-(3-(oxazol-5-yl)benzyl)-6-oxo- 6,8-dihydro-2H,7H- spiro[furo[2,3-e]isoindole-3,4′- piperidin]-7-yl)piperidine-2,6-dione |
| A126 | 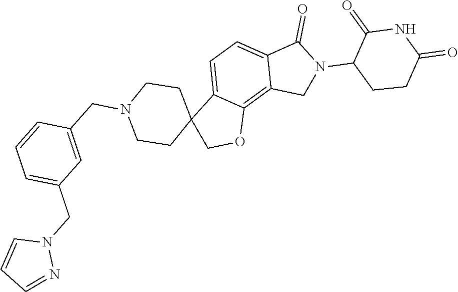 [SMILES: O=C1CCC(N2Cc3c(ccc4c3OCC43CCN(Cc4cccc(Cn5cccn5)c4)CC3)C2=O)C(=O)N1] | 3-(1′-(3-((1H-pyrazol-1- yl)methyl)benzyl)-6-oxo-6,8-dihydro- 2H,7H-spiro[furo[2,3-e]isoindole-3,4′- piperidin]-7-yl)piperidine-2,6-dione |
| A127 | 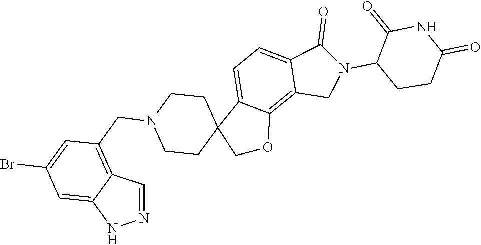 [SMILES: O=C1CCC(N2Cc3c(ccc4c3OCC43CCN(Cc4cc(Br)cc5[nH]ncc45)CC3)C2=O)C(=O)N1] | 3-(1′-((6-bromo-1H-indazol-4- yl)methyl)-6-oxo-6,8-dihydro-2H,7H- spiro[furo[2,3-e]isoindole-3,4′- piperidin]-7-yl)piperidine-2,6-dione |
| A128 | 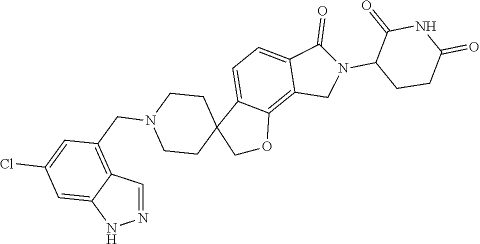 [SMILES: O=C1CCC(N2Cc3c(ccc4c3OCC43CCN(Cc4cc(Cl)cc5[nH]ncc45)CC3)C2=O)C(=O)N1] | 3-(1′-((6-chloro-1H-indazol-4- yl)methyl)-6-oxo-6,8-dihydro-2H,7H- spiro[furo[2,3-e]isoindole-3,4′- piperidin]-7-yl)piperidine-2,6-dione |
| A129 | 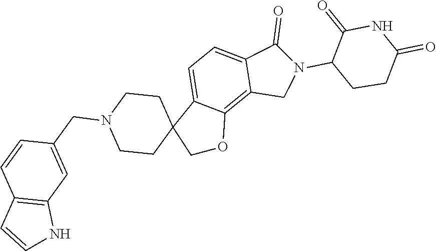 [SMILES: O=C1CCC(N2Cc3c(ccc4c3OCC43CCN(Cc4ccc5cc[nH]c5c4)CC3)C2=O)C(=O)N1] | 3-(1′-((1H-indol-6-yl)methyl)-6-oxo- 6,8-dihydro-2H,7H- spiro[furo[2,3-e]isoindole-3,4′- piperidin]-7-yl)piperidine-2,6-dione |
| A130 | 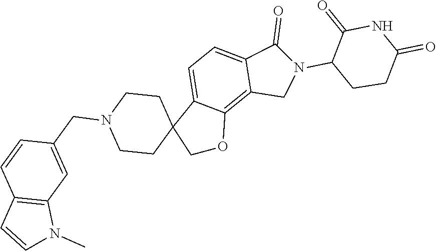 [SMILES: Cn1ccc2ccc(CN3CCC4(CC3)COc3c4ccc4c3CN(C3CCC(=O)NC3=O)C4=O)cc21] | 3-(1′-((1-methyl-1H-indol-6- yl)methyl)-6-oxo-6,8-dihydro-2H,7H- spiro[furo[2,3-e]isoindole-3,4′- piperidin]-7-yl)piperidine-2,6-dione |
| A131 | 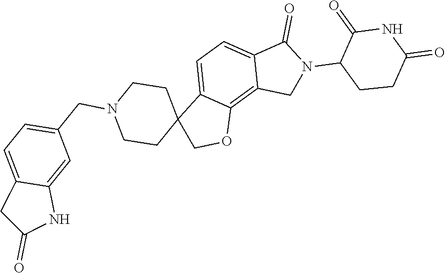 [SMILES: O=C1CCC(N2Cc3c(ccc4c3OCC43CCN(Cc4ccc5c(c4)NC(=O)C5)CC3)C2=O)C(=O)N1] | 3-(6-oxo-1′-((2-oxoindolin-6- yl)methyl)-6,8-dihydro-2H,7H- spiro[furo[2,3-e]isoindole-3,4′- piperidin]-7-yl)piperidine-2,6-dione |
| A132 | 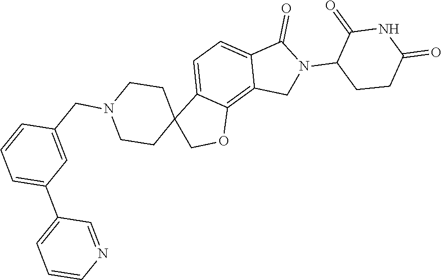 [SMILES: O=C1CCC(N2Cc3c(ccc4c3OCC43CCN(Cc4cccc(-c5cccnc5)c4)CC3)C2=O)C(=O)N1] | 3-(6-oxo-1′-(3-(pyridin-3-yl)benzyl)- 6,8-dihydro-2H,7H- spiro[furo[2,3-e]isoindole-3,4′- piperidin]-7-yl)piperidine-2,6-dione |
| A133 | 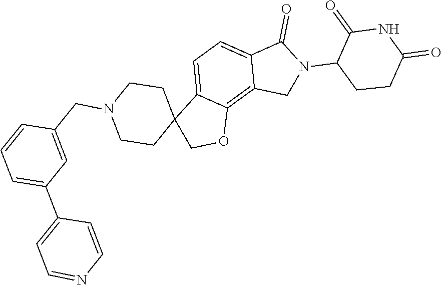 [SMILES: O=C1CCC(N2Cc3c(ccc4c3OCC43CCN(Cc4cccc(-c5ccncc5)c4)CC3)C2=O)C(=O)N1] | 3-(6-oxo-1′-(3-(pyridin-4-yl)benzyl)- 6,8-dihydro-2H,7H- spiro[furo[2,3-e]isoindole-3,4′- piperidin]-7-yl)piperidine-2,6-dione |
| A134 | 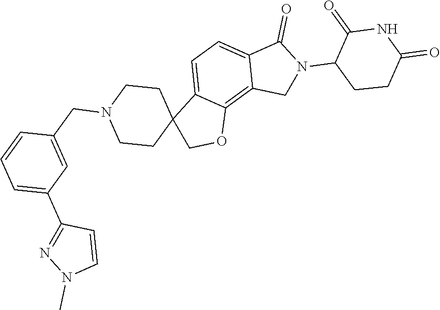 [SMILES: Cn1ccc(-c2cccc(CN3CCC4(CC3)COc3c4ccc4c3CN(C3CCC(=O)NC3=O)C4=O)c2)n1] | 3-(1′-(3-(1-methyl-1H-pyrazol-3- yl)benzyl)-6-oxo-6,8-dihydro-2H,7H- spiro[furo[2,3-e]isoindole-3,4′- piperidin]-7-yl)piperidine-2,6-dione |
| A135 | 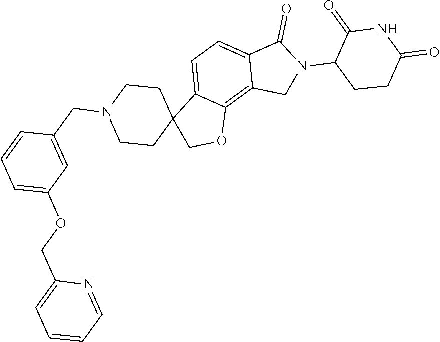 [SMILES: O=C1CCC(N2Cc3c(ccc4c3OCC43CCN(Cc4cccc(OCc5ccccn5)c4)CC3)C2=O)C(=O)N1] | 3-(6-oxo-1′-(3-(pyridin-2- ylmethoxy)benzyl)-6,8-dihydro- 2H,7H-spiro[furo[2,3-e]isoindole-3,4′- piperidin]-7-yl)piperidine-2,6-dione |
| A136 | 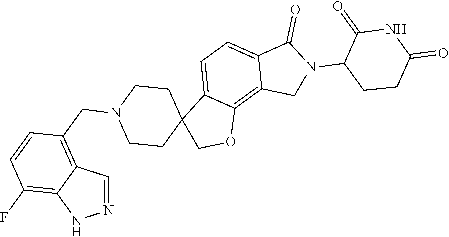 [SMILES: O=C1CCC(N2Cc3c(ccc4c3OCC43CCN(Cc4ccc(F)c5[nH]ncc45)CC3)C2=O)C(=O)N1] | 3-(1′-((7-fluoro-1H-indazol-4- yl)methyl)-6-oxo-6,8-dihydro-2H,7H- spiro[furo[2,3-e]isoindole-3,4′- piperidin]-7-yl)piperidine-2,6-dione |
| A137 | 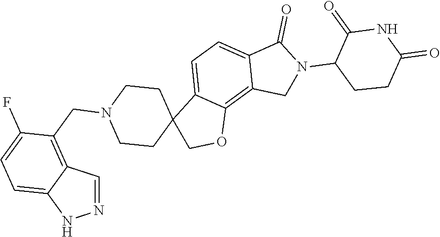 [SMILES: O=C1CCC(N2Cc3c(ccc4c3OCC43CCN(Cc4c(F)ccc5[nH]ncc45)CC3)C2=O)C(=O)N1] | 3-(1′-((5-fluoro-1H-indol-4- yl)methyl)-6-oxo-6,8-dihydro-2H,7H- spiro[furo[2,3-e]isoindole-3,4′- piperidin]-7-yl)piperidine-2,6-dione |
| A138 | 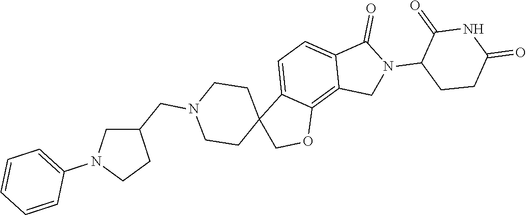 [SMILES: O=C1CCC(N2Cc3c(ccc4c3OCC43CCN(CC4CCN(c5ccccc5)C4)CC3)C2=O)C(=O)N1] | 3-(6-oxo-1′-((1-phenylpyrrolidin-3- yl)methyl)-6,8-dihydro-2H,7H- spiro[furo[2,3-e]isoindole-3,4′- piperidin]-7-yl)piperidine-2,6-dione |
| A139 | 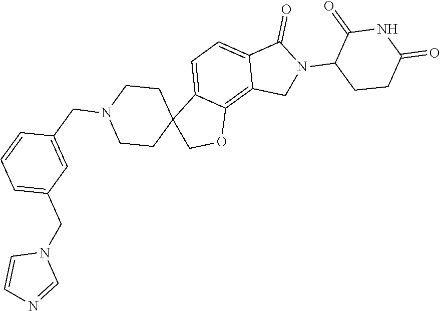 [SMILES: O=C1CCC(N2Cc3c(ccc4c3OCC43CCN(Cc4cccc(Cn5ccnc5)c4)CC3)C2=O)C(=O)N1] | 3-(1′-(3-((1H-imidazol-1- yl)methyl)benzyl)-6-oxo-6,8-dihydro- 2H,7H-spiro[furo[2,3-e]isoindole-3,4′- piperidin]-7-yl)piperidine-2,6-dione |
| A140 | 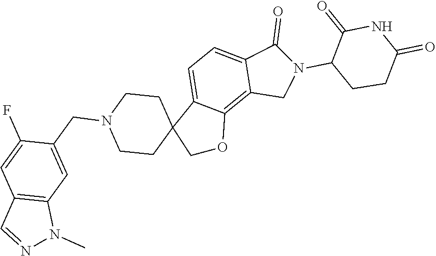 [SMILES: Cn1ncc2cc(F)c(CN3CCC4(CC3)COc3c4ccc4c3CN(C3CCC(=O)NC3=O)C4=O)cc21] | 3-(1′-((5-fluoro-1-methyl-1H-indazol- 6-yl)methyl)-6-oxo-6,8-dihydro- 2H,7H-spiro[furo[2,3-e]isoindole-3,4′- piperidin]-7-yl)piperidine-2,6-dione |
| A141 | 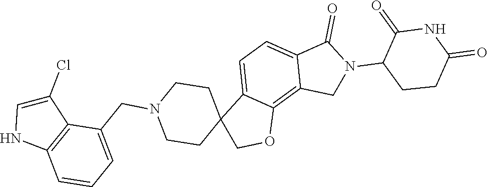 [SMILES: O=C1CCC(N2Cc3c(ccc4c3OCC43CCN(Cc4cccc5[nH]cc(Cl)c45)CC3)C2=O)C(=O)N1] | 3-(1′-((3-chloro-1H-indol-4- yl)methyl)-6-oxo-6,8-dihydro-2H,7H- spiro[furo[2,3-e]isoindole-3,4′- piperidin]-7-yl)piperidine-2,6-dione |
| A142 | 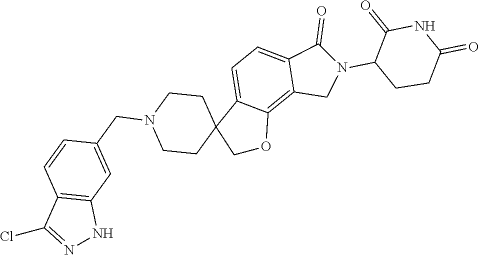 [SMILES: O=C1CCC(N2Cc3c(ccc4c3OCC43CCN(Cc4ccc5c(Cl)n[nH]c5c4)CC3)C2=O)C(=O)N1] | 3-(1′-((3-chloro-1H-indazol-6- yl)methyl)-6-oxo-6,8-dihydro-2H,7H- spiro[furo[2,3-e]isoindole-3,4′- piperidin]-7-yl)piperidine-2,6-dione |
| A143 | 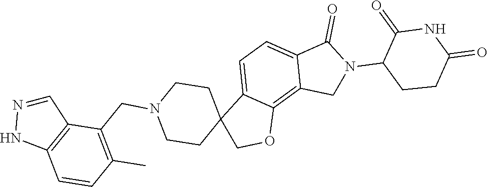 [SMILES: Cc1ccc2[nH]ncc2c1CN1CCC2(CC1)COc1c2ccc2c1CN(C1CCC(=O)NC1=O)C2=O] | 3-(1′-((5-methyl-1H-indazol-4- yl)methyl)-6-oxo-6,8-dihydro-2H,7H- spiro[furo[2,3-e]isoindole-3,4′- piperidin]-7-yl)piperidine-2,6-dione |
| A144 | 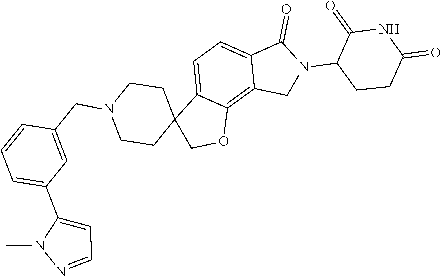 [SMILES: Cn1nccc1-c1cccc(CN2CCC3(CC2)COc2c3ccc3c2CN(C2CCC(=O)NC2=O)C3=O)c1] | 3-(1′-(3-(1-methyl-1H-pyrazol-5- yl)benzyl)-6-oxo-6,8-dihydro-2H,7H- spiro[furo[2,3-e]isoindole-3,4′- piperidin]-7-yl)piperidine-2,6-dione |
| A145 | 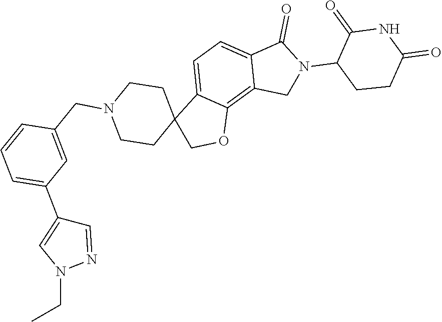 [SMILES: CCn1cc(-c2cccc(CN3CCC4(CC3)COc3c4ccc4c3CN(C3CCC(=O)NC3=O)C4=O)c2)cn1] | 3-(1′-(3-(1-ethyl-1H-pyrazol-4- yl)benzyl)-6-oxo-6,8-dihydro-2H,7H- spiro[furo[2,3-e]isoindole-3,4′- piperidin]-7-yl)piperidine-2,6-dione |
| A146 | 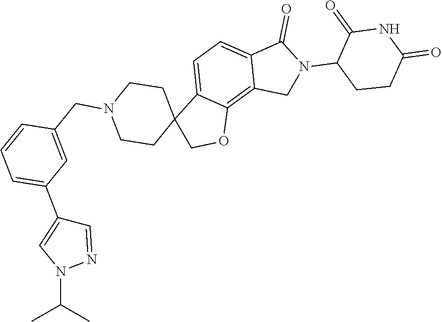 [SMILES: CC(C)n1cc(-c2cccc(CN3CCC4(CC3)COc3c4ccc4c3CN(C3CCC(=O)NC3=O)C4=O)c2)cn1] | 3-(1′-(3-(1-isopropyl-1H-pyrazol-4- yl)benzyl)-6-oxo-6,8-dihydro-2H,7H- spiro[furo[2,3-e]isoindole-3,4′- piperidin]-7-yl)piperidine-2,6-dione |
| A147 | 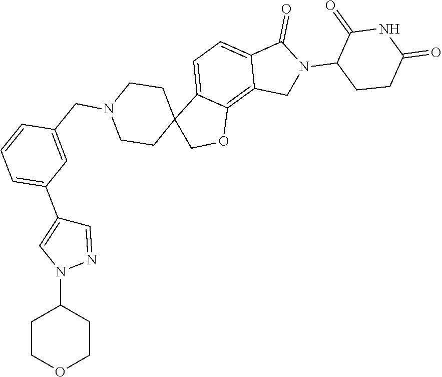 [SMILES: O=C1CCC(N2Cc3c(ccc4c3OCC43CCN(Cc4cccc(-c5cnn(C6CCOCC6)c5)c4)CC3)C2=O)C(=O)N1] | 3-(6-oxo-1′-(3-(1-(tetrahydro-2H- pyran-4-yl)-1H-pyrazol-4-yl)benzyl)- 6,8-dihydro-2H,7H- spiro[furo[2,3-e]isoindole-3,4′- piperidin]-7-yl)piperidine-2,6-dione |
| A148 | 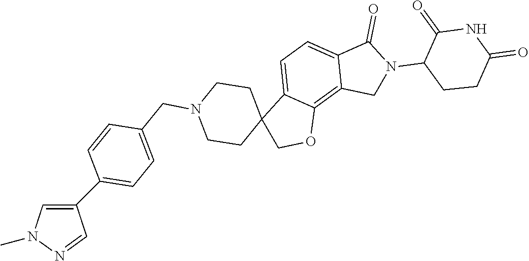 [SMILES: Cn1cc(-c2ccc(CN3CCC4(CC3)COc3c4ccc4c3CN(C3CCC(=O)NC3=O)C4=O)cc2)cn1] | 3-(1′-(4-(1-methyl-1H-pyrazol-4- yl)benzyl)-6-oxo-6,8-dihydro-2H,7H- spiro[furo[2,3-e]isoindole-3,4′- piperidin]-7-yl)piperidine-2,6-dione |
| A149 | 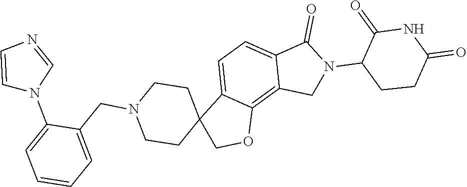 [SMILES: O=C1CCC(N2Cc3c(ccc4c3OCC43CCN(Cc4ccccc4-n4ccnc4)CC3)C2=O)C(=O)N1] | 3-(1′-(2-(1H-imidazol-1-yl)benzyl)-6- oxo-6,8-dihydro-2H,7H- spiro[furo[2,3-e]isoindole-3,4′- piperidin]-7-yl)piperidine-2,6-dione |
| A150 | 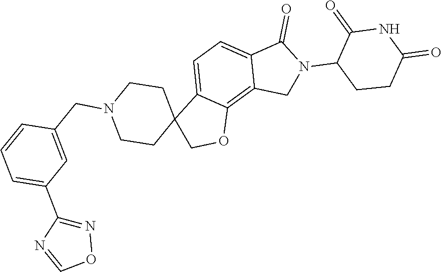 [SMILES: O=C1CCC(N2Cc3c(ccc4c3OCC43CCN(Cc4cccc(-c5ncon5)c4)CC3)C2=O)C(=O)N1] | 3-(1′-(3-(1,2,4-oxadiazol-3-yl)benzyl)- 6-oxo-6,8-dihydro-2H,7H- spiro[furo[2,3-e]isoindole-3,4′- piperidin]-7-yl)piperidine-2,6-dione |
| A151 | 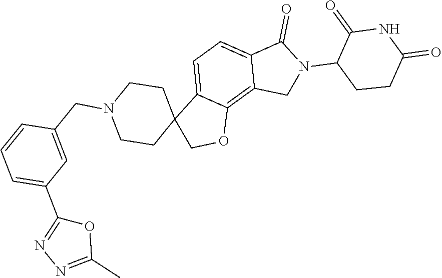 [SMILES: Cc1nnc(-c2cccc(CN3CCC4(CC3)COc3c4ccc4c3CN(C3CCC(=O)NC3=O)C4=O)c2)o1] | 3-(1′-(3-(5-methyl-1,3,4-oxadiazol-2- yl)benzyl)-6-oxo-6,8-dihydro-2H,7H- spiro[furo[2,3-e]isoindole-3,4′- piperidin]-7-yl)piperidine-2,6-dione |
| A152 | 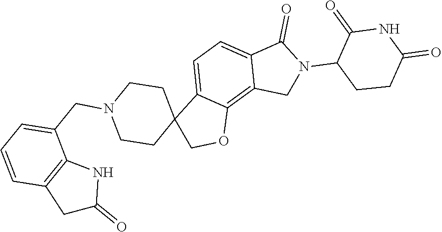 [SMILES: O=C1CCC(N2Cc3c(ccc4c3OCC43CCN(Cc4cccc5c4NC(=O)C5)CC3)C2=O)C(=O)N1] | 3-(6-oxo-1′-((2-oxoindolin-7- yl)methyl)-6,8-dihydro-2H,7H- spiro[furo[2,3-e]isoindole-3,4′- piperidin]-7-yl)piperidine-2,6-dione |
| A153 | 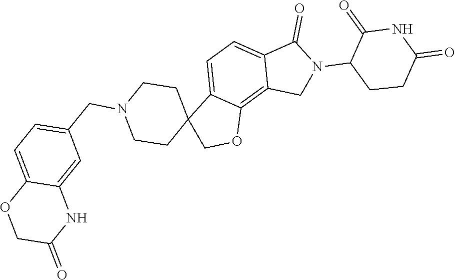 [SMILES: O=C1CCC(N2Cc3c(ccc4c3OCC43CCN(Cc4ccc5c(c4)NC(=O)CO5)CC3)C2=O)C(=O)N1] | 3-(6-oxo-1′-((3-oxo-3,4-dihydro-2H- benzo[b][1,4]oxazin-6-yl)methyl)- 6,8-dihydro-2H,7H- spiro[furo[2,3-e]isoindole-3,4′- piperidin]-7-yl)piperidine-2,6-dione |
| A154 | 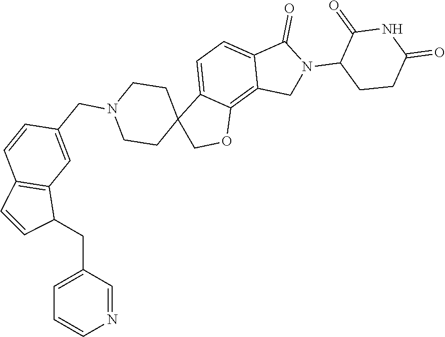 [SMILES: O=C1CCC(N2Cc3c(ccc4c3OCC43CCN(Cc4ccc5c(c4)C(Cc4cccnc4)C=C5)CC3)C2=O)C(=O)N1] | 3-(6-oxo-1′-((1-(pyridin-3-ylmethyl)- 1H-indol-6-yl)methyl)-6,8-dihydro- 2H,7H-spiro[furo[2,3-e]isoindole-3,4′- piperidin]-7-yl)piperidine-2,6-dione |
| A155 | 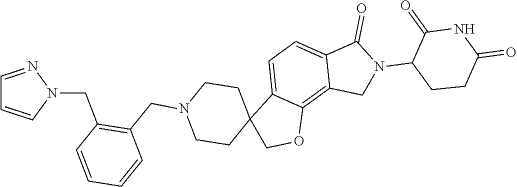 [SMILES: O=C1CCC(N2Cc3c(ccc4c3OCC43CCN(Cc4ccccc4Cn4cccn4)CC3)C2=O)C(=O)N1] | 3-(1′-(2-((1H-pyrazol-1- yl)methyl)benzyl)-6-oxo-6,8-dihydro- 2H,7H-spiro[furo[2,3-e]isoindole-3,4′- piperidin]-7-yl)piperidine-2,6-dione |
| A156 | 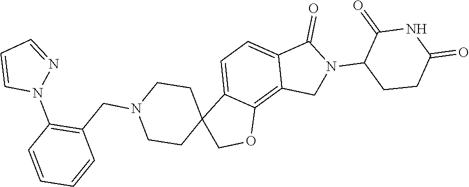 [SMILES: O=C1CCC(N2Cc3c(ccc4c3OCC43CCN(Cc4ccccc4-n4cccn4)CC3)C2=O)C(=O)N1] | 3-(1′-(2-(1H-pyrazol-1-yl)benzyl)-6- oxo-6,8-dihydro-2H,7H- spiro[furo[2,3-e]isoindole-3,4′- piperidin]-7-yl)piperidine-2,6-dione |
| A157 | 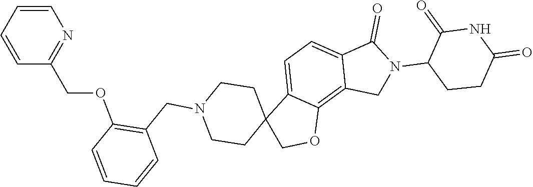 [SMILES: O=C1CCC(N2Cc3c(ccc4c3OCC43CCN(Cc4ccccc4OCc4ccccn4)CC3)C2=O)C(=O)N1] | 3-(6-oxo-1′-(2-(pyridin-2- ylmethoxy)benzyl)-6,8-dihydro- 2H,7H-spiro[furo[2,3-e]isoindole-3,4′- piperidin]-7-yl)piperidine-2,6-dione |
| A158 | 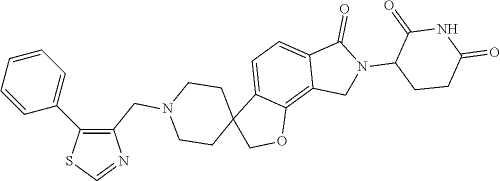 [SMILES: O=C1CCC(N2Cc3c(ccc4c3OCC43CCN(Cc4ncsc4-c4ccccc4)CC3)C2=O)C(=O)N1] | 3-(6-oxo-1′-((5-phenylthiazol-4- yl)methyl)-6,8-dihydro-2H,7H- spiro[furo[2,3-e]isoindole-3,4′- piperidin]-7-yl)piperidine-2,6-dione |
| A159 | 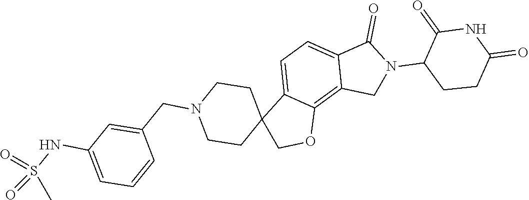 [SMILES: CS(=O)(=O)Nc1cccc(CN2CCC3(CC2)COc2c3ccc3c2CN(C2CCC(=O)NC2=O)C3=O)c1] | N-(3-((7-(2,6-dioxopiperidin-3-yl)-6- oxo-7,8-dihydro-2H,6H- spiro[furo[2,3-e]isoindole-3,4′- piperidin]-1′-yl)methyl)phenyl) methanesulfonamide |
| A160 | 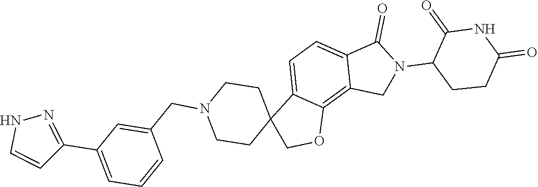 [SMILES: O=C1CCC(N2Cc3c(ccc4c3OCC43CCN(Cc4cccc(-c5cc[nH]n5)c4)CC3)C2=O)C(=O)N1] | 3-(1′-(3-(1H-pyrazol-3-yl)benzyl)-6- oxo-6,8-dihydro-2H,7H- spiro[furo[2,3-e]isoindole-3,4′- piperidin]-7-yl)piperidine-2,6-dione |
| A161 | 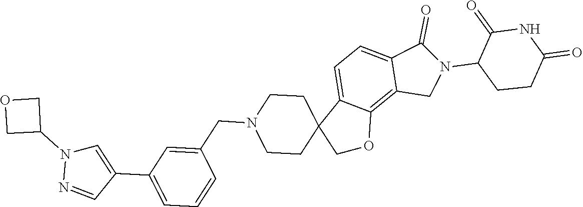 [SMILES: O=C1CCC(N2Cc3c(ccc4c3OCC43CCN(Cc4cccc(-c5cnn(C6COC6)c5)c4)CC3)C2=O)C(=O)N1] | 3-(1′-(3-(1-(oxetan-3-yl)-1H-pyrazol- 4-yl)benzyl)-6-oxo-6,8-dihydro- 2H,7H-spiro[furo[2,3-e]isoindole-3,4′- piperidin]-7-yl)piperidine-2,6-dione |
| A162 | 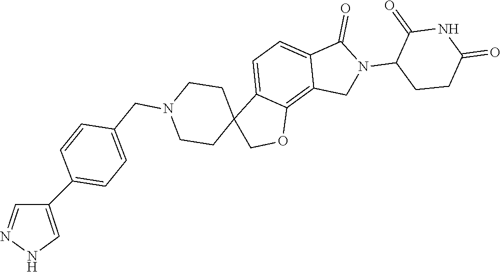 [SMILES: O=C1CCC(N2Cc3c(ccc4c3OCC43CCN(Cc4ccc(-c5cn[nH]c5)cc4)CC3)C2=O)C(=O)N1] | 3-(1′-(4-(1H-pyrazol-4-yl)benzyl)-6- oxo-6,8-dihydro-2H,7H- spiro[furo[2,3-e]isoindole-3,4′- piperidin]-7-yl)piperidine-2,6-dione |
| A163 | 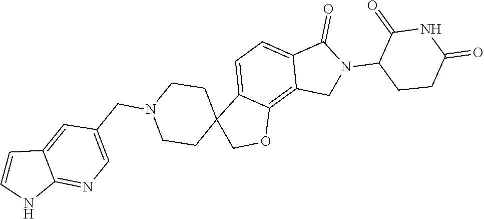 [SMILES: O=C1CCC(N2Cc3c(ccc4c3OCC43CCN(Cc4cnc5[nH]ccc5c4)CC3)C2=O)C(=O)N1] | 3-(1′-((1H-pyrrolo[2,3-b]pyridin-5- yl)methyl)-6-oxo-6,8-dihydro-2H,7H- spiro[furo[2,3-e]isoindole-3,4′- piperidin]-7-yl)piperidine-2,6-dione |
| A164 | 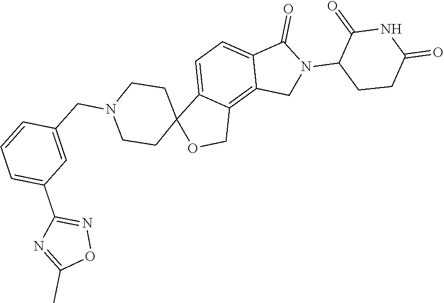 [SMILES: Cc1nc(-c2cccc(CN3CCC4(CC3)OCc3c4ccc4c3CN(C3CCC(=O)NC3=O)C4=O)c2)no1] | 3-(1′-(3-(5-methyl-1,2,4-oxadiazol-3- yl)benzyl)-6-oxo-6,8-dihydro-2H,7H- spiro[furo[2,3-e]isoindole-3,4′- piperidin]-7-yl)piperidine-2,6-dione |
| A165 | 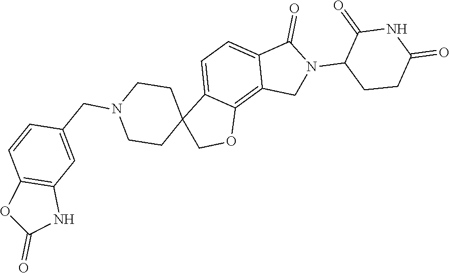 [SMILES: O=C1CCC(N2Cc3c(ccc4c3OCC43CCN(Cc4ccc5oc(=O)[nH]c5c4)CC3)C2=O)C(=O)N1] | 3-(6-oxo-1′-((2-oxo-2,3- dihydrobenzo[d]oxazol-5-yl)methyl)- 6,8-dihydro-2H,7H- spiro[furo[2,3-e]isoindole-3,4′- piperidin]-7-yl)piperidine-2,6-dione |
| A166 | 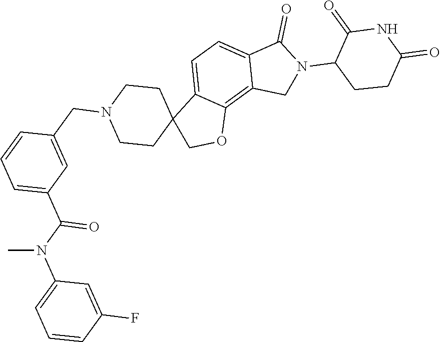 [SMILES: CN(C(=O)c1cccc(CN2CCC3(CC2)COc2c3ccc3c2CN(C2CCC(=O)NC2=O)C3=O)c1)c1cccc(F)c1] | 3-((7-(2,6-dioxopiperidin-3-yl)-6-oxo- 7,8-dihydro-2H,6H- spiro[furo[2,3-e]isoindole-3,4′- piperidin]-1′-yl)methyl)-N-(3- fluorophenyl)-N-methylbenzamide |
| A167 | 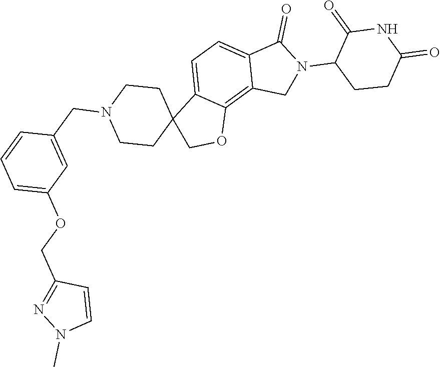 [SMILES: Cn1ccc(COc2cccc(CN3CCC4(CC3)COc3c4ccc4c3CN(C3CCC(=O)NC3=O)C4=O)c2)n1] | 3-(1′-(3-((1-methyl-1H-pyrazol-3- yl)methoxy)benzyl)-6-oxo-6,8- dihydro-2H,7H- spiro[furo[2,3-e]isoindole-3,4′- piperidin]-7-yl)piperidine-2,6-dione |
| A168 | 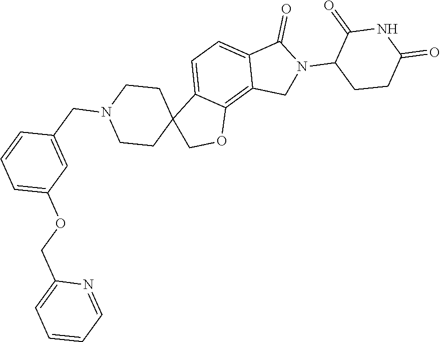 [SMILES: O=C1CCC(N2Cc3c(ccc4c3OCC43CCN(Cc4cccc(OCc5ccccn5)c4)CC3)C2=O)C(=O)N1] | 3-(6-oxo-1′-(3-((pyridin-2- yloxy)methyl)benzyl)-6,8-dihydro- 2H,7H-spiro[furo[2,3-e]isoindole-3,4′- piperidin]-7-yl)piperidine-2,6-dione |
| A169 | 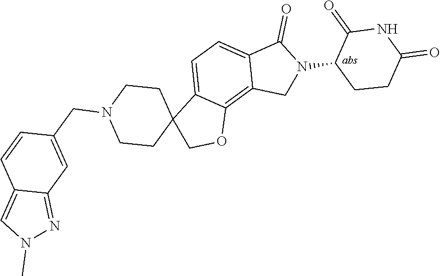 [SMILES: Cn1cc2ccc(CN3CCC4(CC3)COc3c4ccc4c3CN([C@H]3CCC(=O)NC3=O)C4=O)cc2n1] | (S)-3-(1′-((2-methyl-2H-indazol-6- yl)methyl)-6-oxo-6,8-dihydro-2H,7H- spiro[furo[2,3-e]isoindole-3,4′- piperidin]-7-yl)piperidine-2,6-dione |
| A170 | 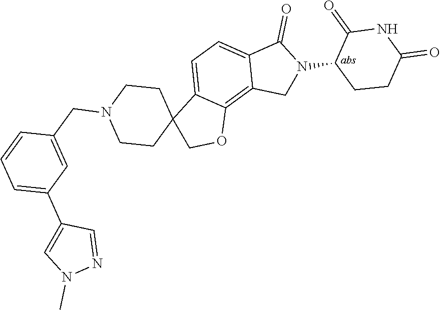 [SMILES: Cn1cc(-c2cccc(CN3CCC4(CC3)COc3c4ccc4c3CN([C@H]3CCC(=O)NC3=O)C4=O)c2)cn1] | (S)-3-(1′-(3-(1-methyl-1H-pyrazol-4- yl)benzyl)-6-oxo-6,8-dihydro-2H,7H- spiro[furo[2,3-e]isoindole-3,4′- piperidin]-7-yl)piperidine-2,6-dione |
| A171 | 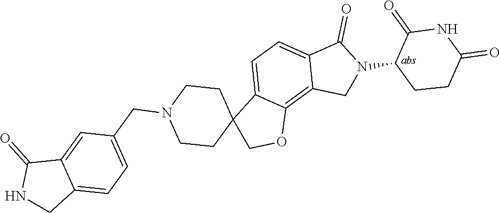 [SMILES: O=C1CC[C@H](N2Cc3c(ccc4c3OCC43CCN(Cc4ccc5c(c4)C(=O)NC5)CC3)C2=O)C(=O)N1] | (S)-3-(6-oxo-1′-((3-oxoisoindolin-5- yl)methyl)-6,8-dihydro-2H,7H- spiro[furo[2,3-e]isoindole-3,4′- piperidin]-7-yl)piperidine-2,6-dione |
| A172 | 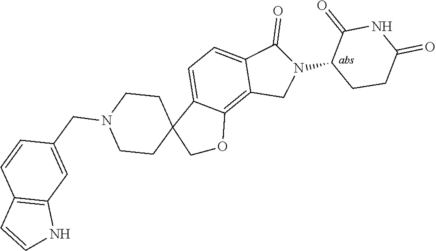 [SMILES: O=C1CC[C@H](N2Cc3c(ccc4c3OCC43CCN(Cc4ccc5cc[nH]c5c4)CC3)C2=O)C(=O)N1] | (S)-3-(1′-((1H-indol-6-yl)methyl)-6- oxo-6,8-dihydro-2H,7H- spiro[furo[2,3-e]isoindole-3,4′- piperidin]-7-yl)piperidine-2,6-dione |
| A173 | 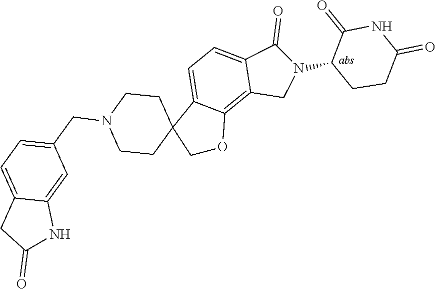 [SMILES: O=C1CC[C@H](N2Cc3c(ccc4c3OCC43CCN(Cc4ccc5c(c4)NC(=O)C5)CC3)C2=O)C(=O)N1] | (S)-3-(6-oxo-1′-((2-oxoindolin-6- yl)methyl)-6,8-dihydro-2H,7H- spiro[furo[2,3-e]isoindole-3,4′- piperidin]-7-yl)piperidine-2,6-dione |
| A174 | 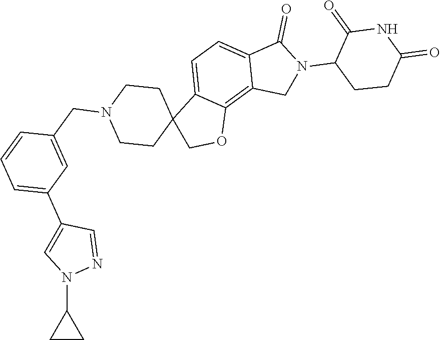 [SMILES: O=C1CCC(N2Cc3c(ccc4c3OCC43CCN(Cc4cccc(-c5cnn(C6CC6)c5)c4)CC3)C2=O)C(=O)N1] | 3-(1′-(3-(1-cyclopropyl-1H-pyrazol-4- yl)benzyl)-6-oxo-6,8-dihydro-2H,7H- spiro[furo[2,3-e]isoindole-3,4′- piperidin]-7-yl)piperidine-2,6-dione |
| A175 | 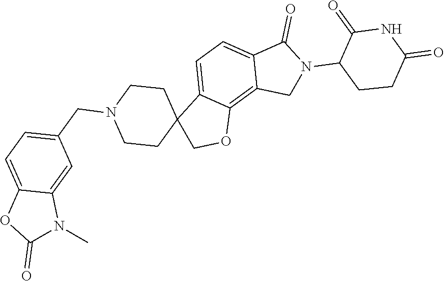 [SMILES: Cn1c(=O)oc2ccc(CN3CCC4(CC3)COc3c4ccc4c3CN(C3CCC(=O)NC3=O)C4=O)cc21] | 3-(1′-((3-methyl-2-oxo-2,3- dihydrobenzo[d]oxazol-5-yl)methyl)- 6-oxo-6,8-dihydro-2H,7H- spiro[furo[2,3-e]isoindole-3,4′- piperidin]-7-yl)piperidine-2,6-dione |
| A176 | 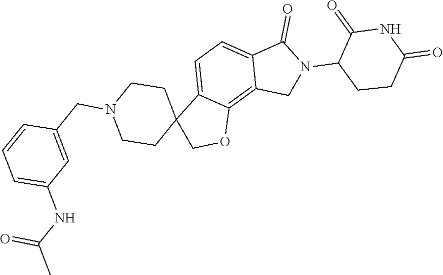 [SMILES: CC(=O)Nc1cccc(CN2CCC3(CC2)COc2c3ccc3c2CN(C2CCC(=O)NC2=O)C3=O)c1] | N-(3-((7-(2,6-dioxopiperidin-3-yl)-6- oxo-7,8-dihydro-2H,6H- spiro[furo[2,3-e]isoindole-3,4′- piperidin]-1′-yl)methyl)phenyl) acetamide |
| A177 | 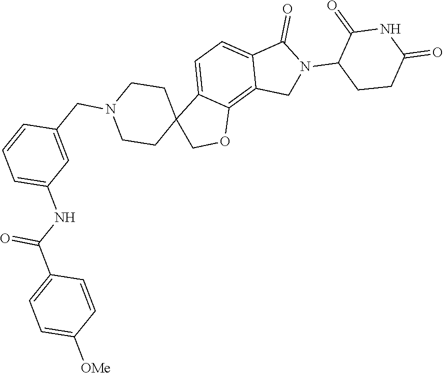 [SMILES: COc1ccc(C(=O)Nc2cccc(CN3CCC4(CC3)COc3c4ccc4c3CN(C3CCC(=O)NC3=O)C4=O)c2)cc1] | N-(3-((7-(2,6-dioxopiperidin-3-yl)-6- oxo-7,8-dihydro-2H,6H- spiro[furo[2,3-e]isoindole-3,4′- piperidin]-1′-yl)methyl)phenyl)-4- methoxybenzamide |
| A178 | 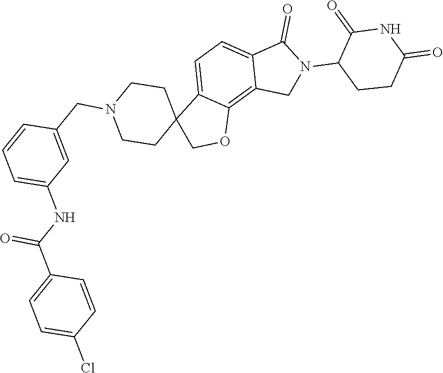 [SMILES: O=C1CCC(N2Cc3c(ccc4c3OCC43CCN(Cc4cccc(NC(=O)c5ccc(Cl)cc5)c4)CC3)C2=O)C(=O)N1] | 4-chloro-N-(3-((7-(2,6-dioxopiperidin- 3-yl)-6-oxo-7,8-dihydro-2H,6H- spiro[furo[2,3-e]isoindole-3,4′- piperidin]-1′- yl)methyl)phenyl)benzamide |
| A179 | 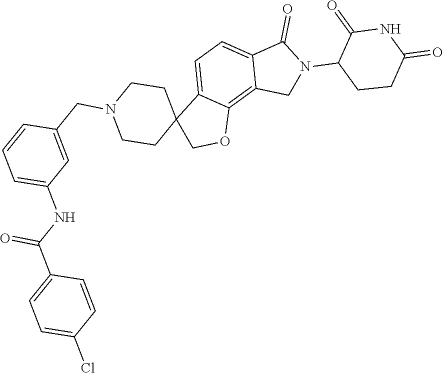 [SMILES: O=C1CCC(N2Cc3c(ccc4c3OCC43CCN(Cc4cccc(NC(=O)c5ccc(Cl)cc5)c4)CC3)C2=O)C(=O)N1] | N-(3-((7-(2,6-dioxopiperidin-3-yl)-6- oxo-7,8-dihydro-2H,6H- spiro[furo[2,3-e]isoindole-3,4′- piperidin]-1′-yl)methyl)phenyl)-4- methylbenzamide |
| A180 | 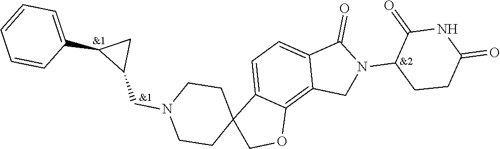 [SMILES: O=C1CCC(N2Cc3c(ccc4c3OCC43CCN(C[C@H]4C[C@@H]4c4ccccc4)CC3)C2=O)C(=O)N1] | 3-(6-oxo-1′-((trans-2- phenylcyclopropyl)methyl)-6,8- dihydro-2H,7H- spiro[furo[2,3-e]isoindole-3,4′- piperidin]-7-yl)piperidine-2,6-dione |
| A181 | 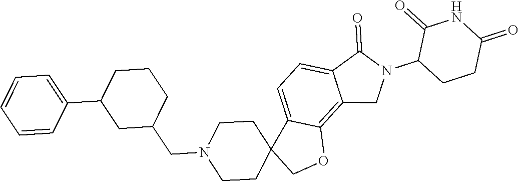 [SMILES: O=C1CCC(N2Cc3c(ccc4c3OCC43CCN(CC4CCCC(c5ccccc5)C4)CC3)C2=O)C(=O)N1] | 3-(6-oxo-1′-((3- phenylcyclohexyl)methyl)-6,8- dihydro-2H,7H- spiro[furo[2,3-e]isoindole-3,4′- piperidin]-7-yl)piperidine-2,6-dione |
| A182 | 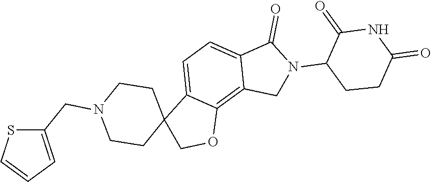 [SMILES: O=C1CCC(N2Cc3c(ccc4c3OCC43CCN(Cc4cccs4)CC3)C2=O)C(=O)N1] | 3-(6-oxo-1′-(thiophen-2-ylmethyl)- 6,8-dihydro-2H,7H- spiro[furo[2,3-e]isoindole-3,4′- piperidin]-7-yl)piperidine-2,6-dione |
| A183 | 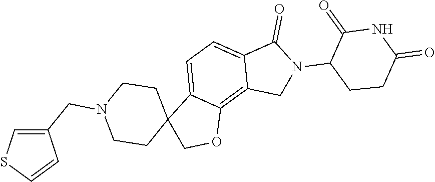 [SMILES: O=C1CCC(N2Cc3c(ccc4c3OCC43CCN(Cc4ccsc4)CC3)C2=O)C(=O)N1] | 3-(6-oxo-1′-(thiophen-3-ylmethyl)- 6,8-dihydro-2H,7H- spiro[furo[2,3-e]isoindole-3,4′- piperidin]-7-yl)piperidine-2,6-dione |
| A184 | 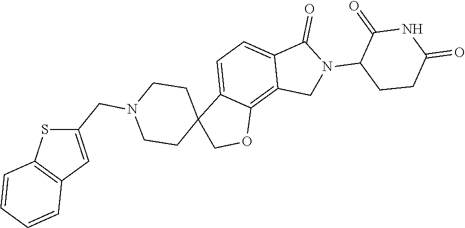 [SMILES: O=C1CCC(N2Cc3c(ccc4c3OCC43CCN(Cc4cc5ccccc5s4)CC3)C2=O)C(=O)N1] | 3-(1′-(benzo[b]thiophen-2-ylmethyl)- 6-oxo-6,8-dihydro-2H,7H- spiro[furo[2,3-e]isoindole-3,4′- piperidin]-7-yl)piperidine-2,6-dione |
| A185 | 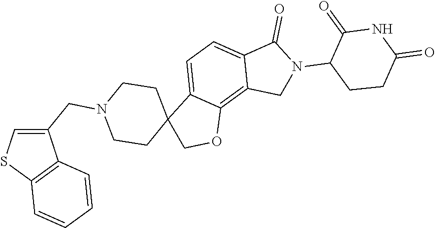 [SMILES: O=C1CCC(N2Cc3c(ccc4c3OCC43CCN(Cc4csc5ccccc45)CC3)C2=O)C(=O)N1] | 3-(1′-(benzo[b]thiophen-3-ylmethyl)- 6-oxo-6,8-dihydro-2H,7H- spiro[furo[2,3-e]isoindole-3,4′- piperidin]-7-yl)piperidine-2,6-dione |
| A186 | 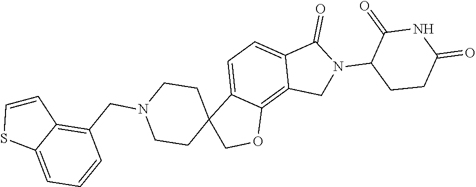 [SMILES: O=C1CCC(N2Cc3c(ccc4c3OCC43CCN(Cc4cccc5sccc45)CC3)C2=O)C(=O)N1] | 3-(1′-(benzo[b]thiophen-4-ylmethyl)- 6-oxo-6,8-dihydro-2H,7H- spiro[furo[2,3-e]isoindole-3,4′- piperidin]-7-yl)piperidine-2,6-dione |
| A187 | 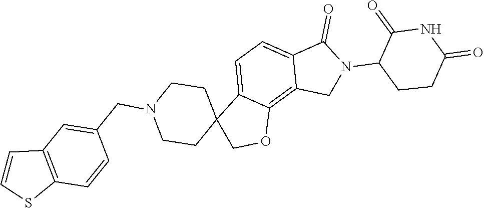 [SMILES: O=C1CCC(N2Cc3c(ccc4c3OCC43CCN(Cc4ccc5sccc5c4)CC3)C2=O)C(=O)N1] | 3-(1′-(benzo[b]thiophen-6-ylmethyl)- 6-oxo-6,8-dihydro-2H,7H- spiro[furo[2,3-e]isoindole-3,4′- piperidin]-7-yl)piperidine-2,6-dione |
| A188 | 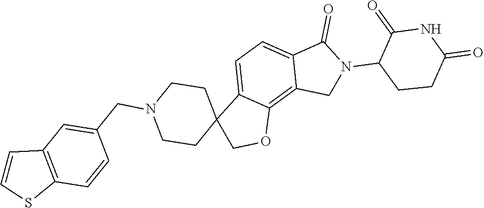 [SMILES: O=C1CCC(N2Cc3c(ccc4c3OCC43CCN(Cc4ccc5sccc5c4)CC3)C2=O)C(=O)N1] | 3-(1′-(benzo[b]thiophen-7-ylmethyl)- 6-oxo-6,8-dihydro-2H,7H- spiro[furo[2,3-e]isoindole-3,4′- piperidin]-7-yl)piperidine-2,6-dione |
| A189 | 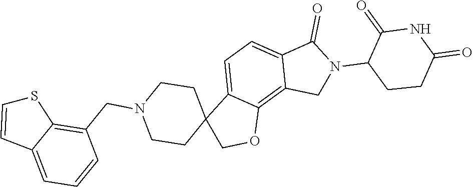 [SMILES: O=C1CCC(N2Cc3c(ccc4c3OCC43CCN(Cc4cccc5ccsc45)CC3)C2=O)C(=O)N1] | 3-(1′-(benzo[b]thiophen-7-ylmethyl)- 6-oxo-6,8-dihydro-2H,7H- spiro[furo[2,3-e]isoindole-3,4′- piperidin]-7-yl)piperidine-2,6-dione |
| A190 | 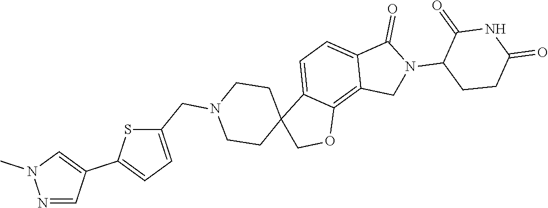 [SMILES: Cn1cc(-c2ccc(CN3CCC4(CC3)COc3c4ccc4c3CN(C3CCC(=O)NC3=O)C4=O)s2)cn1] | 3-(1′-((5-(1-methyl-1H-pyrazol-4- yl)thiophen-2-yl)methyl)-6-oxo-6,8- dihydro-2H,7H- spiro[furo[2,3-e]isoindole-3,4′- piperidin]-7-yl)piperidine-2,6-dione |
| A191 | 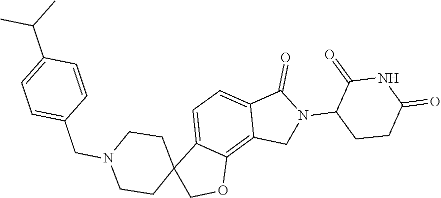 [SMILES: CC(C)c1ccc(CN2CCC3(CC2)COc2c3ccc3c2CN(C2CCC(=O)NC2=O)C3=O)cc1] | 3-(1′-(4-isopropylbenzyl)-6-oxo-6,8- dihydro-2H,7H- spiro[furo[2,3-e]isoindole-3,4′- piperidin]-7-yl)piperidine-2,6-dione |
| A192 | 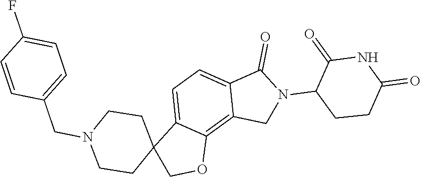 [SMILES: O=C1CCC(N2Cc3c(ccc4c3OCC43CCN(Cc4ccc(F)cc4)CC3)C2=O)C(=O)N1] | 3-(1′-(4-fluorobenzyl)-6-oxo-6,8- dihydro-2H,7H- spiro[furo[2,3-e]isoindole-3,4′- piperidin]-7-yl)piperidine-2,6-dione |
| A193 | 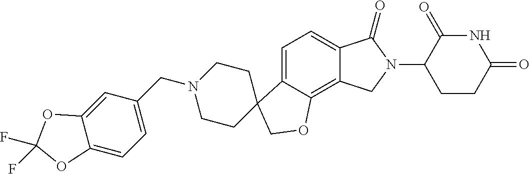 [SMILES: O=C1CCC(N2Cc3c(ccc4c3OCC43CCN(Cc4ccc5c(c4)OC(F)(F)O5)CC3)C2=O)C(=O)N1] | 3-(1′-((2,2- difluorobenzo[d][1,3]dioxol-5- yl)methyl)-6-oxo-6,8-dihydro-2H,7H- spiro[furo[2,3-e]isoindole-3,4′- piperidin]-7-yl)piperidine-2,6-dione |
| A194 | 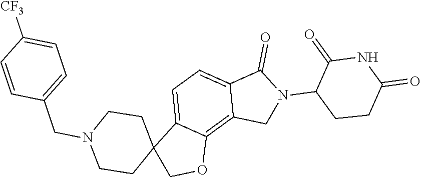 [SMILES: O=C1CCC(N2Cc3c(ccc4c3OCC43CCN(Cc4ccc(C(F)(F)F)cc4)CC3)C2=O)C(=O)N1] | 3-(6-oxo-1′-(4- (trifluoromethyl)benzyl)-6,8-dihydro- 2H,7H-spiro[furo[2,3-e]isoindole-3,4′- piperidin]-7-yl)piperidine-2,6-dione |
| A195 | 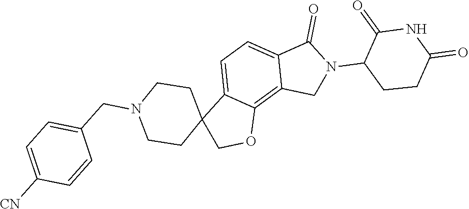 | 4-((7-(2,6-dioxopiperidin-3-yl)-6-oxo- 7,8-dihydro-2H,6H-spiro[furo[2,3- e]isoindole-3,4′-piperidin]-1′- yl)methyl)benzonitrile |
| A196 | 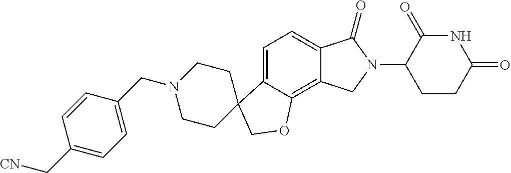 | 2-(4-((7-(2,6-dioxopiperidin-3-yl)-6- oxo-7,8-dihydro-2H,6H- spiro[furo[2,3-e]isoindole-3,4′- piperidin]-1′- yl)methyl)phenyl)acetonitrile |
| A197 | 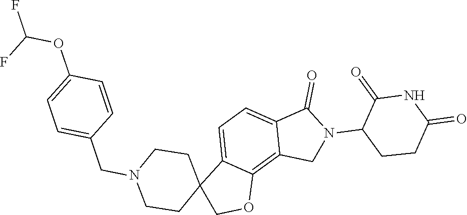 [SMILES: O=C1CCC(N2Cc3c(ccc4c3OCC43CCN(Cc4ccc(OC(F)F)cc4)CC3)C2=O)C(=O)N1] | 3-(1′-(4-(difluoromethoxy)benzyl)-6- oxo-6,8-dihydro-2H,7H- spiro[furo[2,3-e]isoindole-3,4′- piperidin]-7-yl)piperidine-2,6-dione |
| A198 | 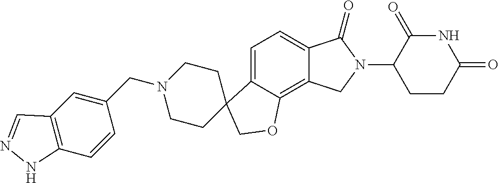 [SMILES: O=C1CCC(N2Cc3c(ccc4c3OCC43CCN(Cc4ccc5[nH]ncc5c4)CC3)C2=O)C(=O)N1] | 3-(1′-((1H-indazol-5-yl)methyl)-6- oxo-6,8-dihydro-2H,7H- spiro[furo[2,3-e]isoindole-3,4′- piperidin]-7-yl)piperidine-2,6-dione |
| A199 | 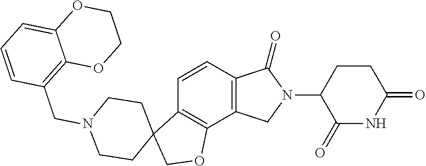 [SMILES: O=C1CCC(N2Cc3c(ccc4c3OCC43CCN(Cc4cccc5c4OCCO5)CC3)C2=O)C(=O)N1] | 3-(1′-((2,3- dihydrobenzo[b][1,4]dioxin-5- yl)methyl)-6-oxo-6,8-dihydro-2H,7H- spiro[furo[2,3-e]isoindole-3,4′- piperidin]-7-yl)piperidine-2,6-dione |
| A200 | 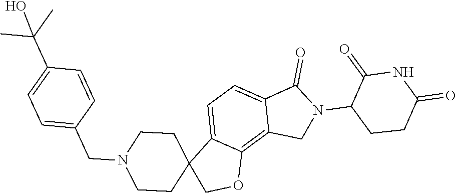 [SMILES: CC(C)(O)c1ccc(CN2CCC3(CC2)COc2c3ccc3c2CN(C2CCC(=O)NC2=O)C3=O)cc1] | 3-(1′-(4-(2-hydroxypropan-2- yl)benzyl)-6-oxo-6,8-dihydro-2H,7H- spiro[furo[2,3-e]isoindole-3,4′- piperidin]-7-yl)piperidine-2,6-dione |
| A201 | 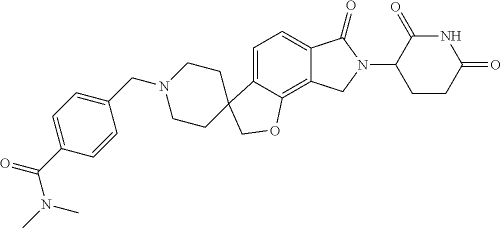 [SMILES: CN(C)C(=O)c1ccc(CN2CCC3(CC2)COc2c3ccc3c2CN(C2CCC(=O)NC2=O)C3=O)cc1] | 4-((7-(2,6-dioxopiperidin-3-yl)-6-oxo- 7,8-dihydro-2H,6H-spiro[furo[2,3- e]isoindole-3,4′-piperidin]-1′- yl)methyl)-N,N-dimethylbenzamide |
| A202 | 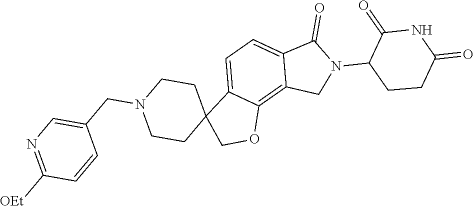 [SMILES: CCOc1ccc(CN2CCC3(CC2)COc2c3ccc3c2CN(C2CCC(=O)NC2=O)C3=O)cn1] | 3-(1′-((6-ethoxypyridin-3-yl)methyl)- 6-oxo-6,8-dihydro-2H,7H- spiro[furo[2,3-e]isoindole-3,4′- piperidin]-7-yl)piperidine-2,6-dione |
| A203 | 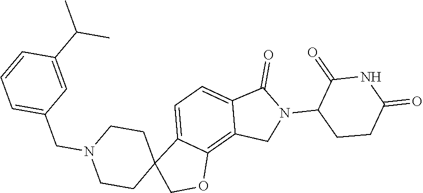 [SMILES: CC(C)c1cccc(CN2CCC3(CC2)COc2c3ccc3c2CN(C2CCC(=O)NC2=O)C3=O)c1] | 3-(1′-(3-isopropylbenzyl)-6-oxo-6,8- dihydro-2H,7H- spiro[furo[2,3-e]isoindole-3,4′- piperidin]-7-yl)piperidine-2,6-dione |
| A204 | 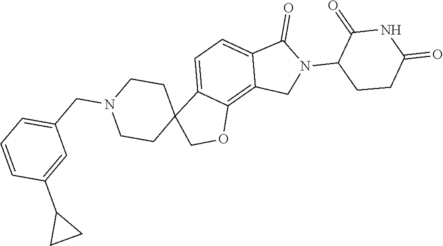 [SMILES: O=C1CCC(N2Cc3c(ccc4c3OCC43CCN(Cc4cccc(C5CC5)c4)CC3)C2=O)C(=O)N1] | 3-(1′-(3-cyclopropylbenzyl)-6-oxo- 6,8-dihydro-2H,7H- spiro[furo[2,3-e]isoindole-3,4′- piperidin]-7-yl)piperidine-2,6-dione |
| A205 | 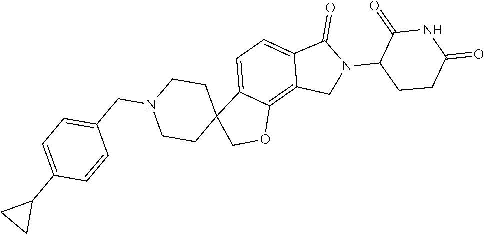 [SMILES: O=C1CCC(N2Cc3c(ccc4c3OCC43CCN(Cc4ccc(C5CC5)cc4)CC3)C2=O)C(=O)N1] | 3-(1′-(4-cyclopropylbenzyl)-6-oxo- 6,8-dihydro-2H,7H- spiro[furo[2,3-e]isoindole-3,4′- piperidin]-7-yl)piperidine-2,6-dione |
| A206 | 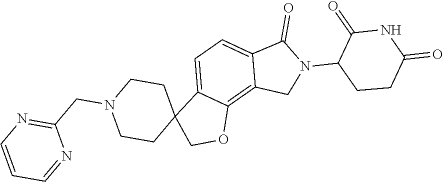 [SMILES: O=C1CCC(N2Cc3c(ccc4c3OCC43CCN(Cc4ncccn4)CC3)C2=O)C(=O)N1] | 3-(6-oxo-1′-(pyrimidin-2-ylmethyl)- 6,8-dihydro-2H,7H- spiro[furo[2,3-e]isoindole-3,4′- piperidin]-7-yl)piperidine-2,6-dione |
| A207 | 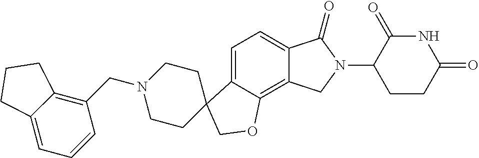 [SMILES: O=C1CCC(N2Cc3c(ccc4c3OCC43CCN(Cc4cccc5c4CCC5)CC3)C2=O)C(=O)N1] | 3-(1′-((2,3-dihydro-1H-inden-4- yl)methyl)-6-oxo-6,8-dihydro-2H,7H- spiro[furo[2,3-e]isoindole-3,4′- piperidin]-7-yl)piperidine-2,6-dione |
| A208 | 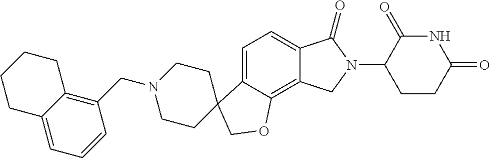 [SMILES: O=C1CCC(N2Cc3c(ccc4c3OCC43CCN(Cc4cccc5c4CCCC5)CC3)C2=O)C(=O)N1] | 3-(6-oxo-1′-((5,6,7,8- tetrahydronaphthalen-1-yl)methyl)- 6,8-dihydro-2H,7H- spiro[furo[2,3-e]isoindole-3,4′- piperidin]-7-yl)piperidine-2,6-dione |
| A209 | 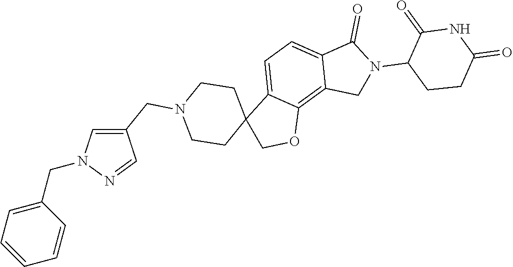 [SMILES: O=C1CCC(N2Cc3c(ccc4c3OCC43CCN(Cc4cnn(Cc5ccccc5)c4)CC3)C2=O)C(=O)N1] | 3-(1′-((1-benzyl-1H-pyrazol-4- yl)methyl)-6-oxo-6,8-dihydro-2H,7H- spiro[furo[2,3-e]isoindole-3,4′- piperidin]-7-yl)piperidine-2,6-dione |
| A210 | 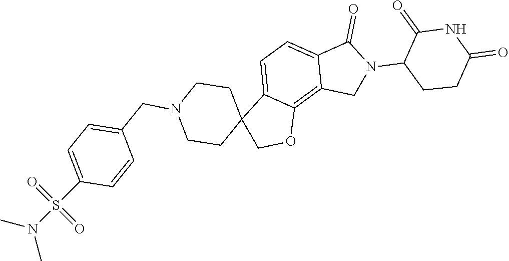 [SMILES: CN(C)S(=O)(=O)c1ccc(CN2CCC3(CC2)COc2c3ccc3c2CN(C2CCC(=O)NC2=O)C3=O)cc1] | 4-((7-(2,6-dioxopiperidin-3-yl)-6-oxo- 7,8-dihydro-2H,6H-spiro[furo[2,3- e]isoindole-3,4′-piperidin]-1′- yl)methyl)-N,N- dimethylbenzenesulfonamide |
| A211 | 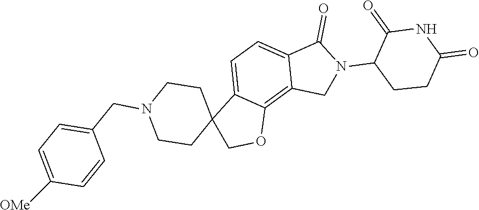 [SMILES: COc1ccc(CN2CCC3(CC2)COc2c3ccc3c2CN(C2CCC(=O)NC2=O)C3=O)cc1] | 3-(1′-(4-methoxybenzyl)-6-oxo-6,8- dihydro-2H,7H- spiro[furo[2,3-e]isoindole-3,4′- piperidin]-7-yl)piperidine-2,6-dione |
| A212 | 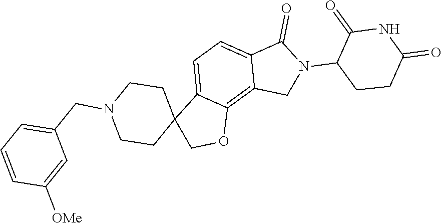 [SMILES: COc1cccc(CN2CCC3(CC2)COc2c3ccc3c2CN(C2CCC(=O)NC2=O)C3=O)c1] | 3-(1′-(3-methoxybenzyl)-6-oxo-6,8- dihydro-2H,7H- spiro[furo[2,3-e]isoindole-3,4′- piperidin]-7-yl)piperidine-2,6-dione |
| A213 | 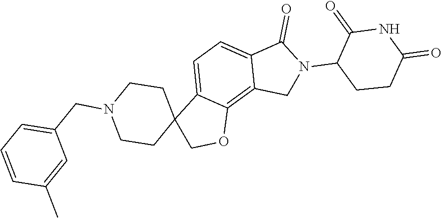 [SMILES: Cc1cccc(CN2CCC3(CC2)COc2c3ccc3c2CN(C2CCC(=O)NC2=O)C3=O)c1] | 3-(1′-(3-methylbenzyl)-6-oxo-6,8- dihydro-2H,7H- spiro[furo[2,3-e]isoindole-3,4′- piperidin]-7-yl)piperidine-2,6-dione |
| A214 | 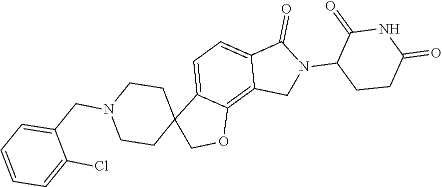 [SMILES: O=C1CCC(N2Cc3c(ccc4c3OCC43CCN(Cc4ccccc4Cl)CC3)C2=O)C(=O)N1] | 3-(1′-(2-chlorobenzyl)-6-oxo-6,8- dihydro-2H,7H- spiro[furo[2,3-e]isoindole-3,4′- piperidin]-7-yl)piperidine-2,6-dione |
| A215 | 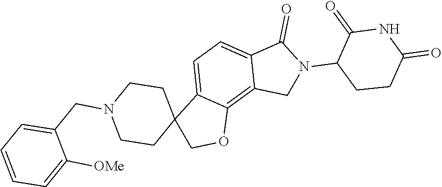 [SMILES: COc1ccccc1CN1CCC2(CC1)COc1c2ccc2c1CN(C1CCC(=O)NC1=O)C2=O] | 3-(1′-(2-methoxybenzyl)-6-oxo-6,8- dihydro-2H,7H- spiro[furo[2,3-e]isoindole-3,4′- piperidin]-7-yl)piperidine-2,6-dione |
| A216 | 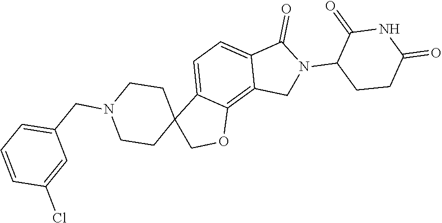 [SMILES: O=C1CCC(N2Cc3c(ccc4c3OCC43CCN(Cc4cccc(Cl)c4)CC3)C2=O)C(=O)N1] | 3-(1′-(3-chlorobenzyl)-6-oxo-6,8- dihydro-2H,7H- spiro[furo[2,3-e]isoindole-3,4′- piperidin]-7-yl)piperidine-2,6-dione |
| A217 | 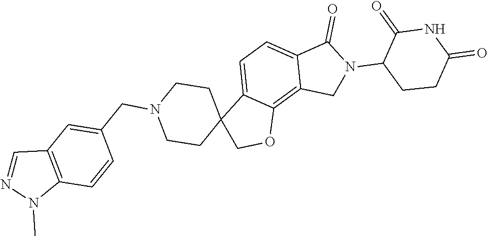 [SMILES: Cn1ncc2cc(CN3CCC4(CC3)COc3c4ccc4c3CN(C3CCC(=O)NC3=O)C4=O)ccc21] | 3-(1′-((1-methyl-1H-indazol-5- yl)methyl)-6-oxo-6,8-dihydro-2H,7H- spiro[furo[2,3-e]isoindole-3,4′- piperidin]-7-yl)piperidine-2,6-dione |
| A218 | 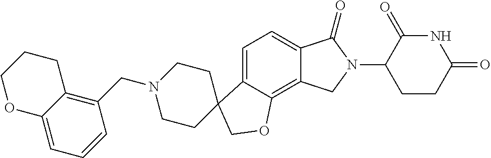 [SMILES: O=C1CCC(N2Cc3c(ccc4c3OCC43CCN(Cc4cccc5c4CCCO5)CC3)C2=O)C(=O)N1] | 3-(1′-(chroman-5-ylmethyl)-6-oxo- 6,8-dihydro-2H,7H- spiro[furo[2,3-e]isoindole-3,4′- piperidin]-7-yl)piperidine-2,6-dione |
| A219 | 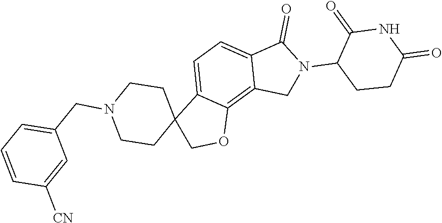 [SMILES: N#Cc1cccc(CN2CCC3(CC2)COc2c3ccc3c2CN(C2CCC(=O)NC2=O)C3=O)c1] | 3-((7-(2,6-dioxopiperidin-3-yl)-6-oxo- 7,8-dihydro-2H,6H-spiro[furo[2,3- e]isoindole-3,4′-piperidin]-1′- yl)methyl)benzonitrile |
| A220 | 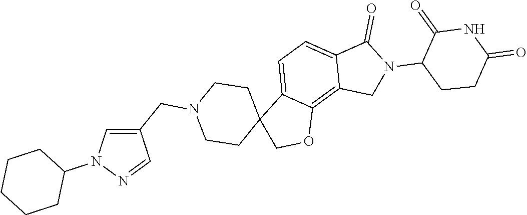 [SMILES: O=C1CCC(N2Cc3c(ccc4c3OCC43CCN(Cc4cnn(C5CCCCC5)c4)CC3)C2=O)C(=O)N1] | 3-(1′-((1-cyclohexyl-1H-pyrazol-4- yl)methyl)-6-oxo-6,8-dihydro-2H,7H- spiro[furo[2,3-e]isoindole-3,4′- piperidin]-7-yl)piperidine-2,6-dione |
| A221 | 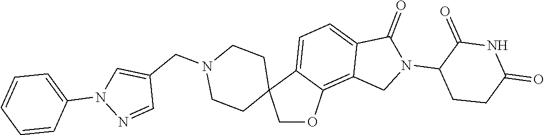 [SMILES: O=C1CCC(N2Cc3c(ccc4c3OCC43CCN(Cc4cnn(-c5ccccc5)c4)CC3)C2=O)C(=O)N1] | 3-(6-oxo-1′-((1-phenyl-1H-pyrazol-4- yl)methyl)-6,8-dihydro-2H,7H- spiro[furo[2,3-e]isoindole-3,4′- piperidin]-7-yl)piperidine-2,6-dione |
| A222 | 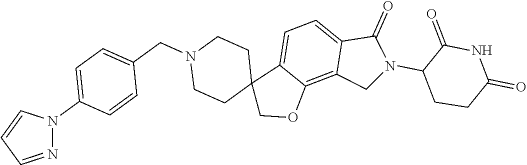 [SMILES: O=C1CCC(N2Cc3c(ccc4c3OCC43CCN(Cc4ccc(-n5cccn5)cc4)CC3)C2=O)C(=O)N1] | 3-(1′-(4-(1H-pyrazol-1-yl)benzyl)-6- oxo-6,8-dihydro-2H,7H- spiro[furo[2,3-e]isoindole-3,4′- piperidin]-7-yl)piperidine-2,6-dione |
| A223 | 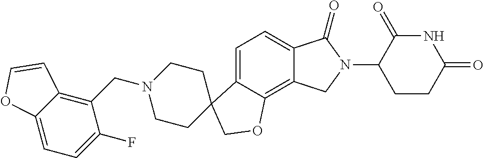 [SMILES: O=C1CCC(N2Cc3c(ccc4c3OCC43CCN(Cc4c(F)ccc5occc45)CC3)C2=O)C(=O)N1] | 3-(1′-((5-fluorobenzofuran-4- yl)methyl)-6-oxo-6,8-dihydro-2H,7H- spiro[furo[2,3-e]isoindole-3,4′- piperidin]-7-yl)piperidine-2,6-dione |
| A224 | 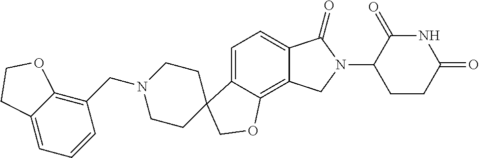 [SMILES: O=C1CCC(N2Cc3c(ccc4c3OCC43CCN(Cc4cccc5c4OCC5)CC3)C2=O)C(=O)N1] | 3-(1′-((2,3-dihydrobenzofuran-7- yl)methyl)-6-oxo-6,8-dihydro-2H,7H- spiro[furo[2,3-e]isoindole-3,4′- piperidin]-7-yl)piperidine-2,6-dione |
| A225 | 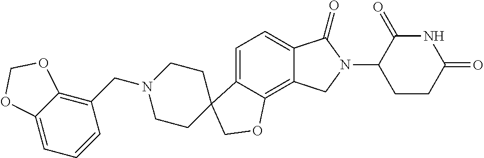 [SMILES: O=C1CCC(N2Cc3c(ccc4c3OCC43CCN(Cc4cccc5c4OCO5)CC3)C2=O)C(=O)N1] | 3-(1′-(benzo[d][1,3]dioxol-4- ylmethyl)-6-oxo-6,8-dihydro-2H,7H- spiro[furo[2,3-e]isoindole-3,4′- piperidin]-7-yl)piperidine-2,6-dione |
| A226 | 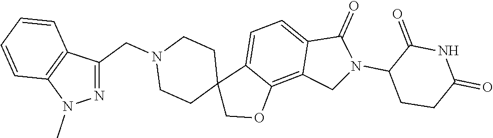 [SMILES: Cn1nc(CN2CCC3(CC2)COc2c3ccc3c2CN(C2CCC(=O)NC2=O)C3=O)c2ccccc21] | 3-(1′-((1-methyl-1H-indazol-3- yl)methyl)-6-oxo-6,8-dihydro-2H,7H- spiro[furo[2,3-e]isoindole-3,4′- piperidin]-7-yl)piperidine-2,6-dione |
| A227 | 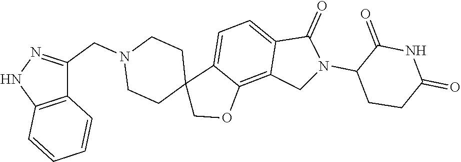 [SMILES: O=C1CCC(N2Cc3c(ccc4c3OCC43CCN(Cc4n[nH]c5ccccc45)CC3)C2=O)C(=O)N1] | 3-(1′-((1H-indazol-3-yl)methyl)-6- oxo-6,8-dihydro-2H,7H- spiro[furo[2,3-e]isoindole-3,4′- piperidin]-7-yl)piperidine-2,6-dione |
| A228 | 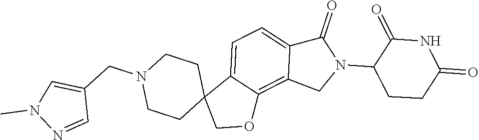 [SMILES: Cn1cc(CN2CCC3(CC2)COc2c3ccc3c2CN(C2CCC(=O)NC2=O)C3=O)cn1] | 3-(1′-((1-methyl-1H-pyrazol-4- yl)methyl)-6-oxo-6,8-dihydro-2H,7H- spiro[furo[2,3-e]isoindole-3,4′- piperidin]-7-yl)piperidine-2,6-dione |
| A229 | 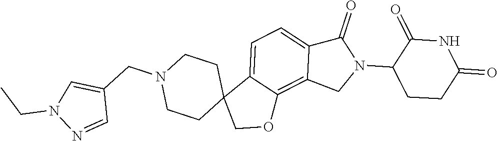 [SMILES: CCn1cc(CN2CCC3(CC2)COc2c3ccc3c2CN(C2CCC(=O)NC2=O)C3=O)cn1] | 3-(1′-((1-ethyl-1H-pyrazol-4- yl)methyl)-6-oxo-6,8-dihydro-2H,7H- spiro[furo[2,3-e]isoindole-3,4′- piperidin]-7-yl)piperidine-2,6-dione |
| A230 | 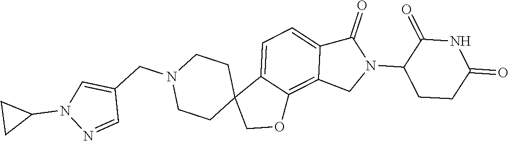 [SMILES: O=C1CCC(N2Cc3c(ccc4c3OCC43CCN(Cc4cnn(C5CC5)c4)CC3)C2=O)C(=O)N1] | 3-(1′-((1-cyclopropyl-1H-pyrazol-4- yl)methyl)-6-oxo-6,8-dihydro-2H,7H- spiro[furo[2,3-e]isoindole-3,4′- piperidin]-7-yl)piperidine-2,6-dione |
| A231 | 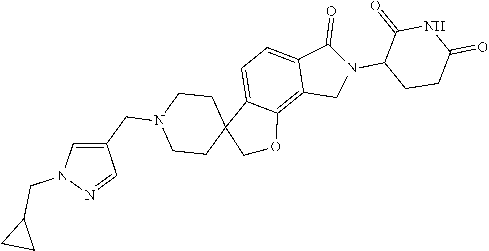 [SMILES: O=C1CCC(N2Cc3c(ccc4c3OCC43CCN(Cc4cnn(CC5CC5)c4)CC3)C2=O)C(=O)N1] | 3-(1′-((1-(cyclopropylmethyl)-1H- pyrazol-4-yl)methyl)-6-oxo-6,8- dihydro-2H,7H- spiro[furo[2,3-e]isoindole-3,4′- piperidin]-7-yl)piperidine-2,6-dione |
| A232 | 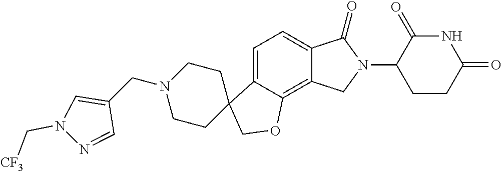 [SMILES: O=C1CCC(N2Cc3c(ccc4c3OCC43CCN(Cc4cnn(CC(F)(F)F)c4)CC3)C2=O)C(=O)N1] | 3-(6-oxo-1′-((1-(2,2,2-trifluoroethyl)- 1H-pyrazol-4-yl)methyl)-6,8-dihydro- 2H,7H-spiro[furo[2,3-e]isoindole-3,4′- piperidin]-7-yl)piperidine-2,6-dione |
| A233 | 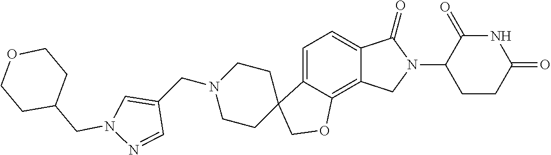 [SMILES: O=C1CCC(N2Cc3c(ccc4c3OCC43CCN(Cc4cnn(CC5CCOCC5)c4)CC3)C2=O)C(=O)N1] | 3-(6-oxo-1′-((1-((tetrahydro-2H- pyran-4-yl)methyl)-1H-pyrazol-4- yl)methyl)-6,8-dihydro-2H,7H- spiro[furo[2,3-e]isoindole-3,4′- piperidin]-7-yl)piperidine-2,6-dione |
| A234 | 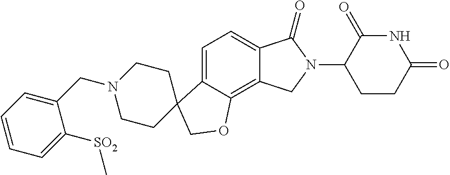 [SMILES: CS(=O)(=O)c1ccccc1CN1CCC2(CC1)COc1c2ccc2c1CN(C1CCC(=O)NC1=O)C2=O] | 3-(1′-(2-(methylsulfonyl)benzyl)-6- oxo-6,8-dihydro-2H,7H- spiro[furo[2,3-e]isoindole-3,4′- piperidin]-7-yl)piperidine-2,6-dione |
| A235 | 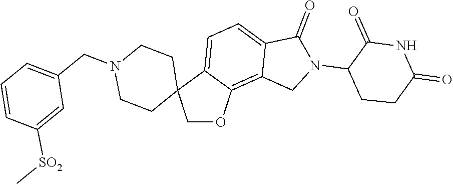 [SMILES: CS(=O)(=O)c1cccc(CN2CCC3(CC2)COc2c3ccc3c2CN(C2CCC(=O)NC2=O)C3=O)c1] | 3-(1′-(3-(methylsulfonyl)benzyl)-6- oxo-6,8-dihydro-2H,7H- spiro[furo[2,3-e]isoindole-3,4′- piperidin]-7-yl)piperidine-2,6-dione |
| A236 | 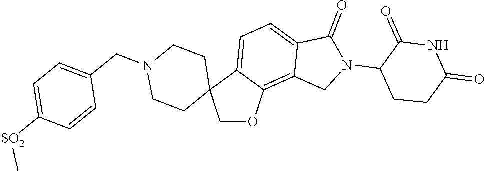 [SMILES: CS(=O)(=O)c1ccc(CN2CCC3(CC2)COc2c3ccc3c2CN(C2CCC(=O)NC2=O)C3=O)cc1] | 3-(1′-(4-(methylsulfonyl)benzyl)-6- oxo-6,8-dihydro-2H,7H- spiro[furo[2,3-e]isoindole-3,4′- piperidin]-7-yl)piperidine-2,6-dione |
| A237 | 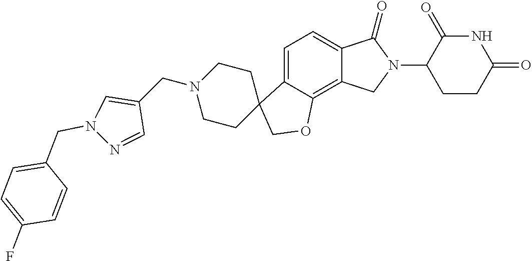 [SMILES: O=C1CCC(N2Cc3c(ccc4c3OCC43CCN(Cc4cnn(Cc5ccc(F)cc5)c4)CC3)C2=O)C(=O)N1] | 3-(1′-((1-(4-fluorobenzyl)-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A238 | 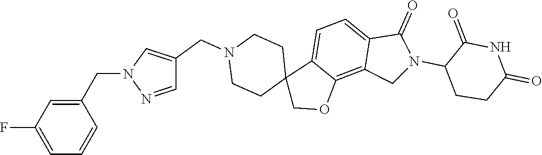 [SMILES: O=C1CCC(N2Cc3c(ccc4c3OCC43CCN(Cc4cnn(Cc5cccc(F)c5)c4)CC3)C2=O)C(=O)N1] | 3-(1′-((1-(3-fluorobenzyl)-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A239 | 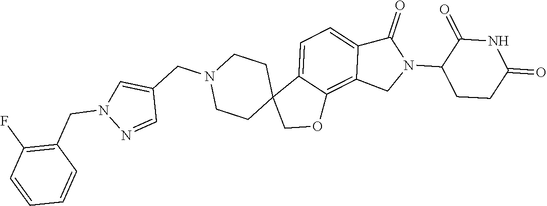 [SMILES: O=C1CCC(N2Cc3c(ccc4c3OCC43CCN(Cc4cnn(Cc5ccccc5F)c4)CC3)C2=O)C(=O)N1] | 3-(1′-((1-(2-fluorobenzyl)-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A240 | 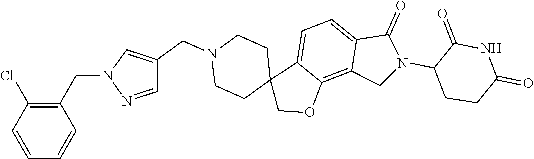 [SMILES: O=C1CCC(N2Cc3c(ccc4c3OCC43CCN(Cc4cnn(Cc5ccccc5Cl)c4)CC3)C2=O)C(=O)N1] | 3-(1′-((1-(2-chlorobenzyl)-1H- pyrazol-4-yl)methyl)-6-oxo-6,8- dihydro-2H,7H- spiro[furo[2,3-e]isoindole-3,4′- piperidin]-7-yl)piperidine-2,6-dione |
| A241 | 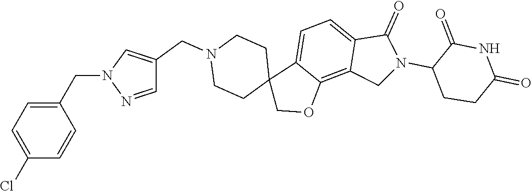 [SMILES: O=C1CCC(N2Cc3c(ccc4c3OCC43CCN(Cc4cnn(Cc5ccc(Cl)cc5)c4)CC3)C2=O)C(=O)N1] | 3-(1′-((1-(4-chlorobenzyl)-1H- pyrazol-4-yl)methyl)-6-oxo-6,8- dihydro-2H,7H- spiro[furo[2,3-e]isoindole-3,4′- piperidin]-7-yl)piperidine-2,6-dione |
| A242 | 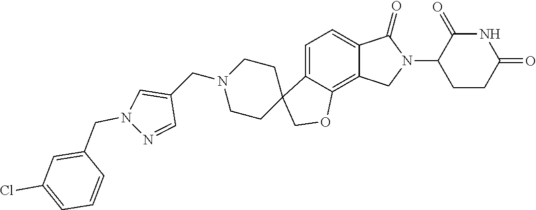 [SMILES: O=C1CCC(N2Cc3c(ccc4c3OCC43CCN(Cc4cnn(Cc5cccc(Cl)c5)c4)CC3)C2=O)C(=O)N1] | 3-(1′-((1-(3-chlorobenzyl)-1H- pyrazol-4-yl)methyl)-6-oxo-6,8- dihydro-2H,7H-spiro[furo[2,3-e] isoindole-3,4′-piperidin]-7-yl) piperidine-2,6-dione |
| A243 | 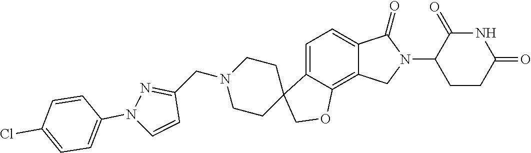 [SMILES: O=C1CCC(N2Cc3c(ccc4c3OCC43CCN(Cc4ccn(-c5ccc(Cl)cc5)n4)CC3)C2=O)C(=O)N1] | 3-(1′-((1-(4-chlorophenyl)-1H- pyrazol-3-yl)methyl)-6-oxo-6,8- dihydro-2H,7H- spiro[furo[2,3-e]isoindole-3,4′- piperidin]-7-yl)piperidine-2,6-dione |
| A244 | 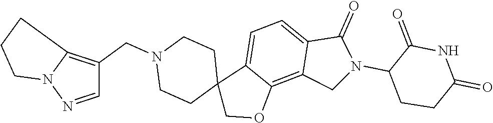 [SMILES: O=C1CCC(N2Cc3c(ccc4c3OCC43CCN(Cc4cnn5c4CCC5)CC3)C2=O)C(=O)N1] | 3-(1′-((5,6-dihydro-4H-pyrrolo[1,2- b]pyrazol-3-yl)methyl)-6-oxo-6,8- dihydro-2H,7H- spiro[furo[2,3-e]isoindole-3,4′- piperidin]-7-yl)piperidine-2,6-dione |
| A245 | 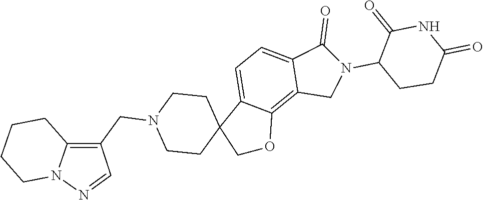 [SMILES: O=C1CCC(N2Cc3c(ccc4c3OCC43CCN(Cc4cnn5c4CCCC5)CC3)C2=O)C(=O)N1] | 3-(6-oxo-1′-((4,5,6,7- tetrahydropyrazolo[1,5-a]pyridin-3- yl)methyl)-6,8-dihydro-2H,7H- spiro[furo[2,3-e]isoindole-3,4′- piperidin]-7-yl)piperidine-2,6-dione |
| A246 | 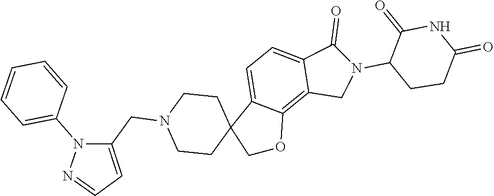 [SMILES: O=C1CCC(N2Cc3c(ccc4c3OCC43CCN(Cc4ccnn4-c4ccccc4)CC3)C2=O)C(=O)N1] | 3-(6-oxo-1′-((1-phenyl-1H-pyrazol-5- yl)methyl)-6,8-dihydro-2H,7H- spiro[furo[2,3-e]isoindole-3,4′- piperidin]-7-yl)piperidine-2,6-dione |
| A247 | 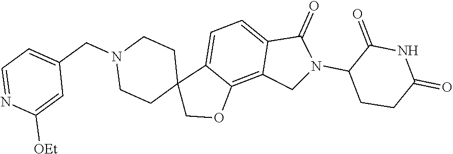 [SMILES: CCOc1cc(CN2CCC3(CC2)COc2c3ccc3c2CN(C2CCC(=O)NC2=O)C3=O)ccn1] | 3-(1′-((2-ethoxypyridin-4-yl)methyl)- 6-oxo-6,8-dihydro-2H,7H- spiro[furo[2,3-e]isoindole-3,4′- piperidin]-7-yl)piperidine-2,6-dione |
| A248 | 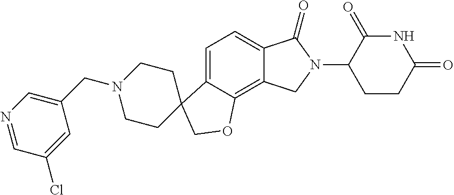 [SMILES: O=C1CCC(N2Cc3c(ccc4c3OCC43CCN(Cc4cncc(Cl)c4)CC3)C2=O)C(=O)N1] | 3-(1′-((5-chloropyridin-3-yl)methyl)- 6-oxo-6,8-dihydro-2H,7H- spiro[furo[2,3-e]isoindole-3,4′- piperidin]-7-yl)piperidine-2,6-dione |
| A249 | 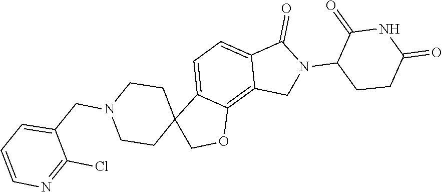 [SMILES: O=C1CCC(N2Cc3c(ccc4c3OCC43CCN(Cc4cccnc4Cl)CC3)C2=O)C(=O)N1] | 3-(1′-((2-chloropyridin-3-yl)methyl)- 6-oxo-6,8-dihydro-2H,7H- spiro[furo[2,3-e]isoindole-3,4′- piperidin]-7-yl)piperidine-2,6-dione |
| A250 | 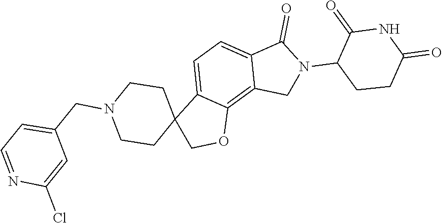 [SMILES: O=C1CCC(N2Cc3c(ccc4c3OCC43CCN(Cc4ccnc(Cl)c4)CC3)C2=O)C(=O)N1] | 3-(1′-((2-chloropyridin-4-yl)methyl)- 6-oxo-6,8-dihydro-2H,7H- spiro[furo[2,3-e]isoindole-3,4′- piperidin]-7-yl)piperidine-2,6-dione |
| A251 | 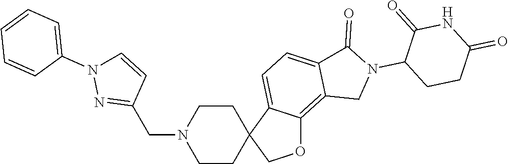 [SMILES: O=C1CCC(N2Cc3c(ccc4c3OCC43CCN(Cc4ccn(-c5ccccc5)n4)CC3)C2=O)C(=O)N1] | 3-(6-oxo-1′-((1-phenyl-1H-pyrazol-3- yl)methyl)-6,8-dihydro-2H,7H- spiro[furo[2,3-e]isoindole-3,4′- piperidin]-7-yl)piperidine-2,6-dione |
| A252 | 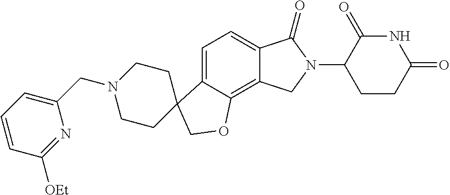 [SMILES: CCOc1cccc(CN2CCC3(CC2)COc2c3ccc3c2CN(C2CCC(=O)NC2=O)C3=O)n1] | 3-(1′-((6-ethoxypyridin-2-yl)methyl)- 6-oxo-6,8-dihydro-2H,7H- spiro[furo[2,3-e]isoindole-3,4′- piperidin]-7-yl)piperidine-2,6-dione |
| A253 | 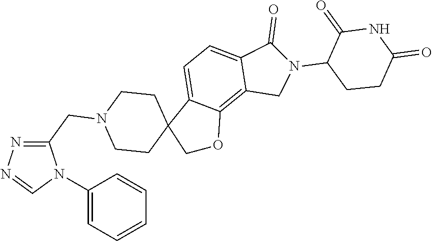 [SMILES: O=C1CCC(N2Cc3c(ccc4c3OCC43CCN(Cc4nncn4-c4ccccc4)CC3)C2=O)C(=O)N1] | 3-(6-oxo-1′-((4-phenyl-4H-1,2,4- triazol-3-yl)methyl)-6,8-dihydro- 2H,7H-spiro[furo[2,3-e]isoindole-3,4′- piperidin]-7-yl)piperidine-2,6-dione |
| A254 | 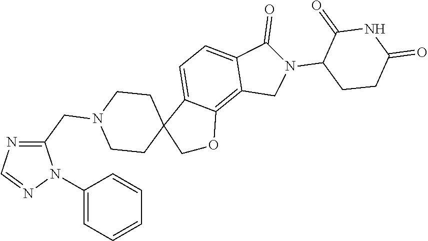 [SMILES: O=C1CCC(N2Cc3c(ccc4c3OCC43CCN(Cc4ncnn4-c4ccccc4)CC3)C2=O)C(=O)N1] | 3-(6-oxo-1′-((1-phenyl-1H-1,2,4- triazol-5-yl)methyl)-6,8-dihydro- 2H,7H-spiro[furo[2,3-e]isoindole-3,4′- piperidin]-7-yl)piperidine-2,6-dione |
| A255 | 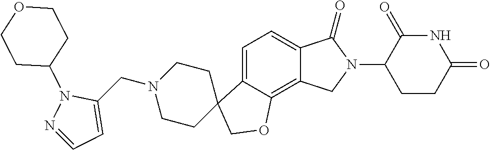 [SMILES: O=C1CCC(N2Cc3c(ccc4c3OCC43CCN(Cc4ccnn4C4CCOCC4)CC3)C2=O)C(=O)N1] | 3-(6-oxo-1′-((1-(tetrahydro-2H-pyran- 4-yl)-1H-pyrazol-5-yl)methyl)-6,8- dihydro-2H,7H- spiro[furo[2,3-e]isoindole-3,4′- piperidin]-7-yl)piperidine-2,6-dione |
| A256 | 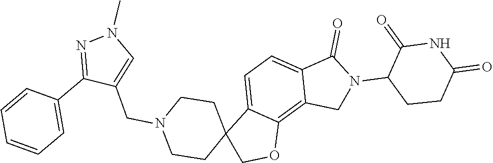 [SMILES: Cn1cc(CN2CCC3(CC2)COc2c3ccc3c2CN(C2CCC(=O)NC2=O)C3=O)c(-c2ccccc2)n1] | 3-(1′-((1-methyl-3-phenyl-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A257 | 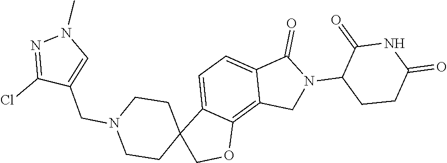 [SMILES: Cn1cc(CN2CCC3(CC2)COc2c3ccc3c2CN(C2CCC(=O)NC2=O)C3=O)c(Cl)n1] | 3-(1′-((3-chloro-1-methyl-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A258 | 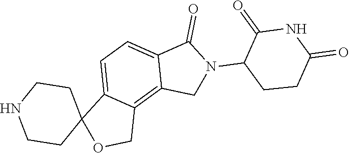 [SMILES: O=C1CCC(N2Cc3c(ccc4c3COC43CCNCC3)C2=O)C(=O)N1] | 3-(6-oxo-6,8-dihydrospiro[furo[3,4- e]isoindole-3,4′-piperidin]-7(1H)- yl)piperidine-2,6-dione |
| A259 | 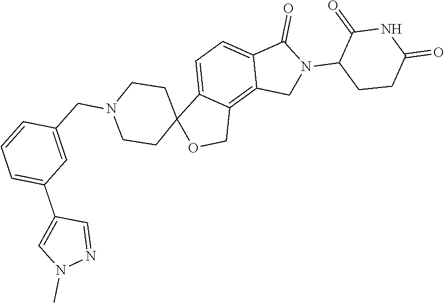 [SMILES: Cn1cc(-c2cccc(CN3CCC4(CC3)OCc3c4ccc4c3CN(C3CCC(=O)NC3=O)C4=O)c2)cn1] | 3-(1′-(3-(1-methyl-1H-pyrazol-4- yl)benzyl)-6-oxo-6,8- dihydrospiro[furo[3,4-e]isoindoline- 3,4′-piperidin]-7(1H)-yl)piperidine- 2,6-dione |
| A260 | 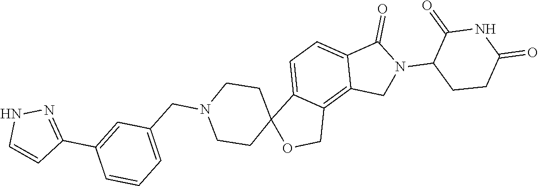 [SMILES: O=C1CCC(N2Cc3c(ccc4c3COC43CCN(Cc4cccc(-c5cc[nH]n5)c4)CC3)C2=O)C(=O)N1] | 3-(1′-(3-(1H-pyrazol-3-yl)benzyl)-6- oxo-6,8-dihydrospiro[furo[3,4-e] isoindoline-3,4′-piperidin]-7(1H)- yl)piperidine-2,6-dione |
| A261 | 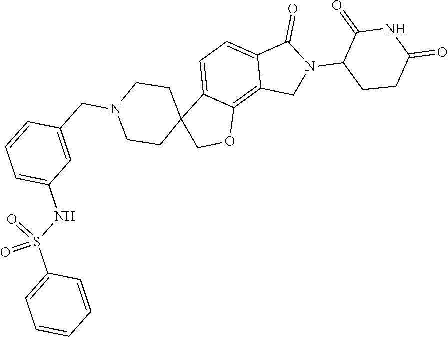 [SMILES: O=C1CCC(N2Cc3c(ccc4c3OCC43CCN(Cc4cccc(NS(=O)(=O)c5ccccc5)c4)CC3)C2=O)C(=O)N1] | N-(3-((7-(2,6-dioxopiperidin-3-yl)-6- oxo-7,8-dihydro-2H,6H- spiro[furo[2,3-e]isoindole-3,4′- piperidin]-1′- yl)methyl)phenyl)benzenesulfonamide |
| A262 | 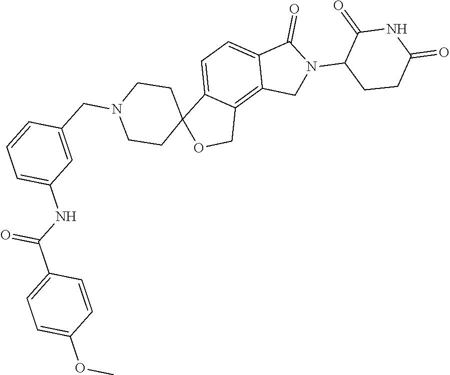 [SMILES: COc1ccc(C(=O)Nc2cccc(CN3CCC4(CC3)OCc3c4ccc4c3CN(C3CCC(=O)NC3=O)C4=O)c2)cc1] | N-(3-((7-(2,6-dioxopiperidin-3-yl)-6- oxo-1,6,7,8-tetrahydrospirofuro [3,4-e]isoindole-3,4′-piperidin]-1′- yl)mehyl)phenyl)-4- methoxybenzamide |
| A263 | 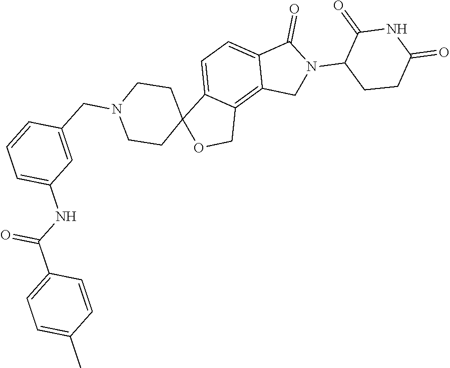 [SMILES: Cc1ccc(C(=O)Nc2cccc(CN3CCC4(CC3)OCc3c4ccc4c3CN(C3CCC(=O)NC3=O)C4=O)c2)cc1] | N-(3-((7-(2,6-dioxopiperidin-3-yl)-6- oxo-1,6,7,8-tetrahydrospiro[furo[3,4- e]isoindole-3,4′-piperidin]-1′- yl)methyl)phenyl)-4-methylbenzamide |
| A264 | 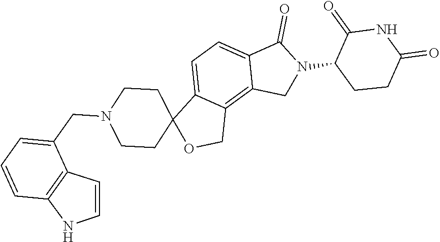 [SMILES: O=C1CC[C@H](N2Cc3c(ccc4c3COC43CCN(Cc4cccc5[nH]ccc45)CC3)C2=O)C(=O)N1] | (S)-3-(1′-((1H-indol-4-yl)methyl)-6- oxo-6,8-dihydro- 2H,7H-spiro[furo[2,3-e]isoindole-3,4′- piperidin]-7-yl)piperidine-2,6-dione |
| A265 | 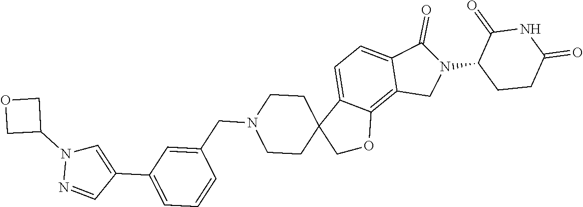 [SMILES: O=C1CC[C@H](N2Cc3c(ccc4c3OCC43CCN(Cc4cccc(-c5cnn(C6COC6)c5)c4)CC3)C2=O)C(=O)N1] | (S)-3-(1′-(3-(1-(oxetan-3-yl)-1H- pyrazol-4-yl)benzyl)-6-oxo-6,8- dihydro-2H,7H-spiro[furo[2,3- e]isoindole-3,4′-piperidin]-7- yl)piperidine-2,6-dione |
| A266 | 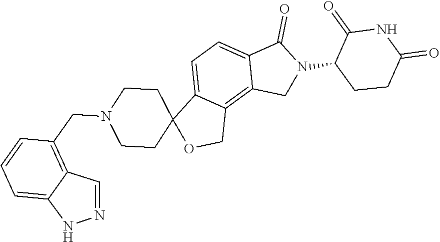 [SMILES: O=C1CC[C@H](N2Cc3c(ccc4c3COC43CCN(Cc4cccc5[nH]ncc45)CC3)C2=O)C(=O)N1] | (S)-3-(1′-((1H-indazol-4-yl)methyl)-6- oxo-6,8-dihydro- 2H,7H-spiro[furo[2,3-e]isoindole-3,4′- piperidin]-7-yl)piperidine-2,6-dione |
| A267 | 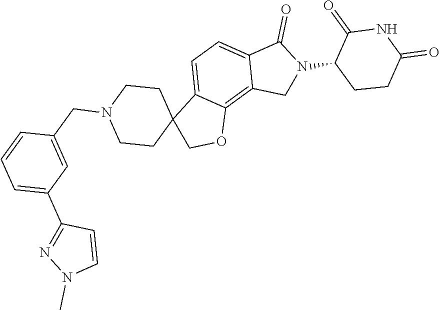 [SMILES: Cn1ccc(-c2cccc(CN3CCC4(CC3)COc3c4ccc4c3CN([C@H]3CCC(=O)NC3=O)C4=O)c2)n1] | (S)-3-(1′-(3-(1-methyl-1H-pyrazol-3- yl)benzyl)-6-oxo-6,8-dihydro- 2H,7H-spiro[furo[2,3-e]isoindole-3,4′- piperidin]-7-yl)piperidine-2,6-dione |
| A268 | 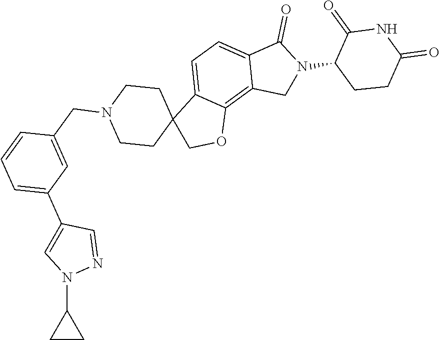 [SMILES: O=C1CC[C@H](N2Cc3c(ccc4c3OCC43CCN(Cc4cccc(-c5cnn(C6CC6)c5)c4)CC3)C2=O)C(=O)N1] | (S)-3-(1′-(3-(1-cyclopropyl-1H- pyrazol-4-yl)benzyl)-6-oxo-6,8- dihydro- 2H,7H-spiro[furo[2,3-e]isoindole-3,4′- piperidin]-7-yl)piperidine-2,6-dione |
| A269 | 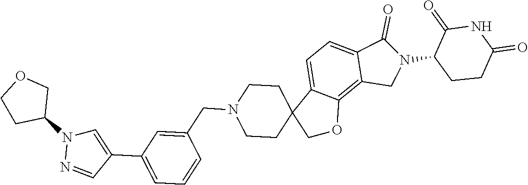 [SMILES: O=C1CC[C@H](N2Cc3c(ccc4c3OCC43CCN(Cc4cccc(-c5cnn([C@H]6CCOC6)c5)c4)CC3)C2=O)C(=O)N1] | (S)-3-(6-oxo-1′-(3-(1-((S)- tetrahydrofuran-3-yl)-1H-pyrazol-4- yl)benzyl)-6,8-dihydro- 2H,7H-spiro[furo[2,3-e]isoindole-3,4′- piperidin]-7-yl)piperidine-2,6-dione |
| A270 | 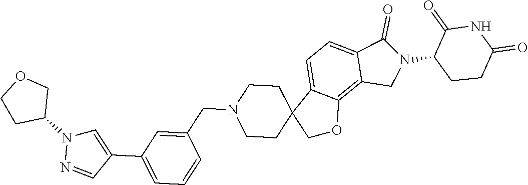 [SMILES: O=C1CC[C@H](N2Cc3c(ccc4c3OCC43CCN(Cc4cccc(-c5cnn([C@@H]6CCOC6)c5)c4)CC3)C2=O)C(=O)N1] | (S)-3-(6-oxo-1′-(3-(1-((R)- tetrahydrofuran-3-yl)-1H-pyrazol-4- yl)benzyl)-6,8-dihydro- 2H,7H-spiro[furo[2,3-e]isoindole-3,4′- piperidin]-7-yl)piperidine-2,6-dione |
| A271 | 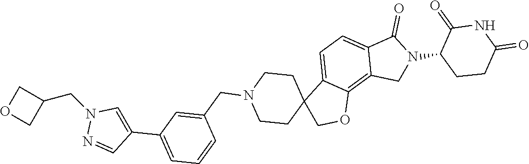 [SMILES: O=C1CC[C@H](N2Cc3c(ccc4c3OCC43CCN(Cc4cccc(-c5cnn(CC6COC6)c5)c4)CC3)C2=O)C(=O)N1] | (S)-3-(1′-(3-(1-(oxetan-3-ylmethyl)- 1H-pyrazol-4-yl)benzyl)-6-oxo-6,8- dihydro- 2H,7H-spiro[furo[2,3-e]isoindole-3,4′- piperidin]-7-yl)piperidine-2,6-dione |
| A272 | 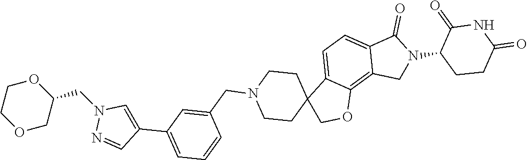 [SMILES: O=C1CC[C@H](N2Cc3c(ccc4c3OCC43CCN(Cc4cccc(-c5cnn(C[C@@H]6COCCO6)c5)c4)CC3)C2=O)C(=O)N1] | (S)-3-(1′-(3-(1-(((R)-1,4-dioxan-2- yl)methyl)-1H-pyrazol-4-yl)benzyl)-6- oxo-6,8-dihydro- 2H,7H-spiro[furo[2,3-e]isoindole-3,4′- piperidin]-7-yl)piperidine-2,6-dione |
| A273 | 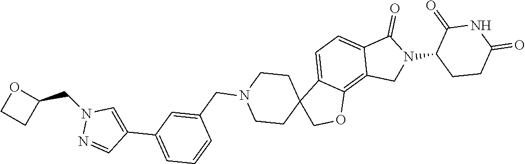 [SMILES: O=C1CC[C@H](N2Cc3c(ccc4c3OCC43CCN(Cc4cccc(-c5cnn(C[C@H]6CCO6)c5)c4)CC3)C2=O)C(=O)N1] | (S)-3-(1′-(3-(1-(((R)-oxetan-2- yl)methyl)-1H-pyrazol-4-yl)benzyl)-6- oxo-6,8-dihydro- 2H,7H-spiro[furo[2,3-e]isoindole-3,4′- piperidin]-7-yl)piperidine-2,6-dione |
| A274 | 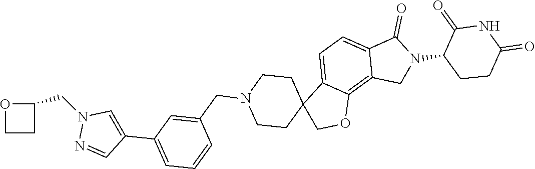 [SMILES: O=C1CC[C@H](N2Cc3c(ccc4c3OCC43CCN(Cc4cccc(-c5cnn(C[C@@H]6CCO6)c5)c4)CC3)C2=O)C(=O)N1] | (S)-3-(1′-(3-(1-(((S)-oxetan-2- yl)methyl)-1H-pyrazol-4-yl)benzyl)-6- oxo-6,8-dihydro- 2H,7H-spiro[furo[2,3-e]isoindole-3,4′- piperidin]-7-yl)piperidine-2,6-dione |
| A275 | 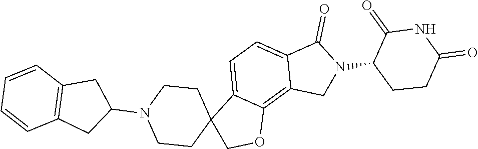 [SMILES: O=C1CC[C@H](N2Cc3c(ccc4c3OCC43CCN(C4Cc5ccccc5C4)CC3)C2=O)C(=O)N1] | (S)-3-(1′-(2,3-dihydro-1H-inden-2-yl)- 6-oxo-6,8-dihydro- 2H,7H-spiro[furo[2,3-e]isoindole-3,4′- piperidin]-7-yl)piperidine-2,6-dione |
| A276 | 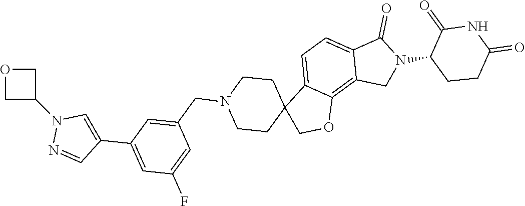 [SMILES: O=C1CC[C@H](N2Cc3c(ccc4c3OCC43CCN(Cc4cc(F)cc(-c5cnn(C6COC6)c5)c4)CC3)C2=O)C(=O)N1] | (S)-3-(1′-(3-fluoro-5-(1-(oxetan-3-yl)- 1H-pyrazol-4-yl)benzyl)-6-oxo-6,8- dihydro- 2H,7H-spiro[furo[2,3-e]isoindole-3,4′- piperidin]-7-yl)piperidine-2,6-dione |
| A277 | 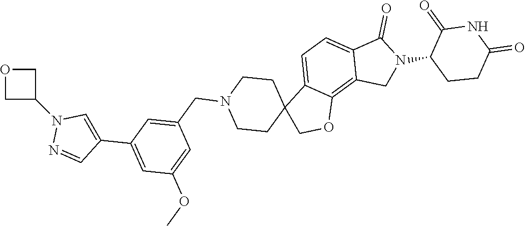 [SMILES: COc1cc(CN2CCC3(CC2)COc2c3ccc3c2CN([C@H]2CCC(=O)NC2=O)C3=O)cc(-c2cnn(C3COC3)c2)c1] | (S)-3-(1′-(3-methoxy-5-(1-oxetan-3- yl)-1H-pyrazol-4-yl)benzyl)-6-oxo- 6,8-dihydro- 2H,7H-spiro[furo[2,3-e]isoindole-3,4′- piperidin]-7-yl)piperidine-2,6-dione |
| A278 | 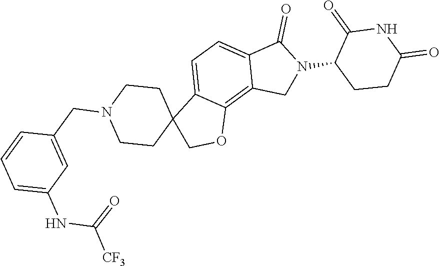 [SMILES: O=C1CC[C@H](N2Cc3c(ccc4c3OCC43CCN(Cc4cccc(NC(=O)C(F)(F)F)c4)CC3)C2=O)C(=O)N1] | (S)-N-(3-((7-(2,6-dioxopiperidin-3- yl)-6-oxo-7,8-dihydro-2H,6H- spiro[furo[2,3-e]isoindole-3,4′- piperidin]-1′-yl)methyl)phenyl)-2,2,2- trifluoroacetamide |
| A279 | 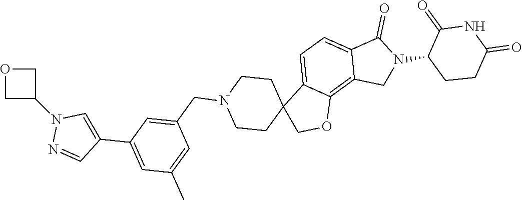 [SMILES: Cc1cc(CN2CCC3(CC2)COc2c3ccc3c2CN([C@H]2CCC(=O)NC2=O)C3=O)cc(-c2cnn(C3COC3)c2)c1] | (S)-3-(1′-(3-methyl-5-(1-(oxetan-3- yl)-1H-pyrazol-4-yl)benzyl)-6-oxo- 6,8-dihydro- 2H,7H-spiro[furo[2,3-e]isoindole-3,4′- piperidin]-7-yl)piperidine-2,6-dione |
| A280 | 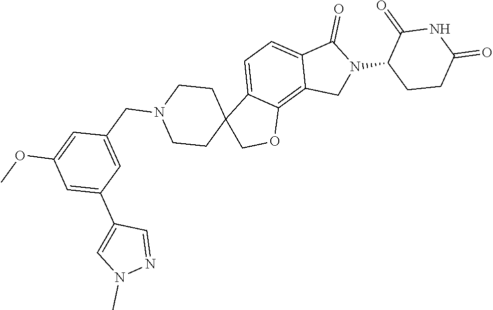 [SMILES: COc1cc(CN2CCC3(CC2)COc2c3ccc3c2CN([C@H]2CCC(=O)NC2=O)C3=O)cc(-c2cnn(C)c2)c1] | (S)-3-(1′-(3-methoxy-5-(1-methyl-1H- pyrazol-4-yl)benzyl)-6-oxo-6,8- dihydro- 2H,7H-spiro[furo[2,3-e]isoindole-3,4′- piperidin]-7-yl)piperidine-2,6-dione |
| A281 | 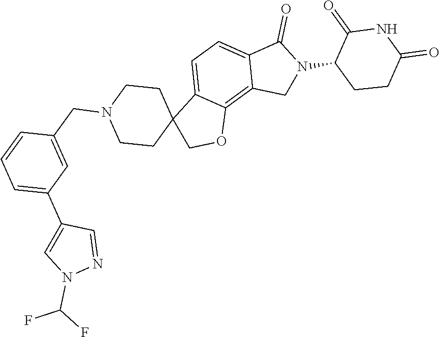 [SMILES: O=C1CC[C@H](N2Cc3c(ccc4c3OCC43CCN(Cc4cccc(-c5cnn(C(F)F)c5)c4)CC3)C2=O)C(=O)N1] | (S)-3-(1′-(3-(1-(difluoromethyl)-1H- pyrazol-4-yl)benzyl)-6-oxo-6,8- dihydro- 2H,7H-spiro[furo[2,3-e]isoindole-3,4′- piperidin]-7-yl)piperidine-2,6-dione |
| A282 | 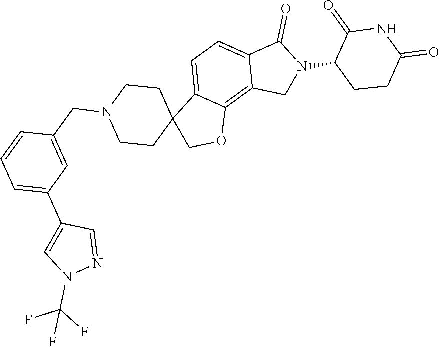 [SMILES: O=C1CC[C@H](N2Cc3c(ccc4c3OCC43CCN(Cc4cccc(-c5cnn(C(F)(F)F)c5)c4)CC3)C2=O)C(=O)N1] | (S)-3-(6-oxo-1′-(3-(1- (trifluoromethyl)-1H-pyrazol-4- yl)benzyl)-6,8-dihydro- 2H,7H-spiro[furo[2,3-e]isoindole-3,4′- piperidin]-7-yl)piperidine-2,6-dione |
| A283 | 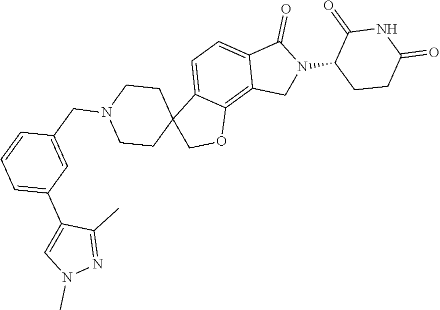 [SMILES: Cc1nn(C)cc1-c1cccc(CN2CCC3(CC2)COc2c3ccc3c2CN([C@H]2CCC(=O)NC2=O)C3=O)c1] | (S)-3-(1′-(3-(1,3-dimethyl-1H-pyrazol- 4-yl)benzyl)-6-oxo-6,8-dihydro- 2H,7H-spiro[furo[2,3-e]isoindole-3,4′- piperidin]-7-yl)piperidine-2,6-dione |
| A284 | 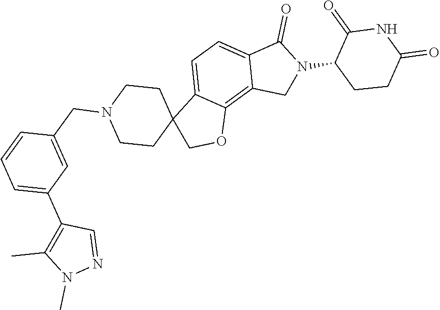 [SMILES: Cc1c(-c2cccc(CN3CCC4(CC3)COc3c4ccc4c3CN([C@H]3CCC(=O)NC3=O)C4=O)c2)cnn1C] | (S)-3-(1′-(3-(1,5-dimethyl-1H-pyrazol- 4-yl)benzyl)-6-oxo-6,8-dihydro- 2H,7H-spiro[furo[2,3-e]isoindole-3,4′- piperidin]-7-yl)piperidine-2,6-dione |
| A285 | 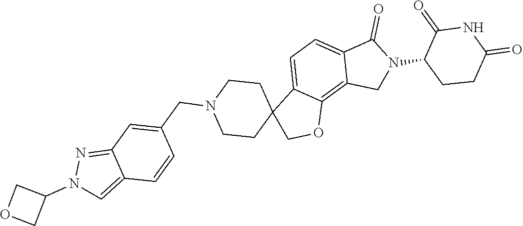 [SMILES: O=C1CC[C@H](N2Cc3c(ccc4c3OCC43CCN(Cc4ccc5cn(C6COC6)nc5c4)CC3)C2=O)C(=O)N1] | (S)-3-(1′-((2-(oxetan-3-yl)-2H- indazol-6-yl)methyl)-6-oxo-6,8- dihydro- 2H,7H-spiro[furo[2,3-e]isoindole-3,4′- piperidin]-7-yl)piperidine-2,6-dione |
| A286 | 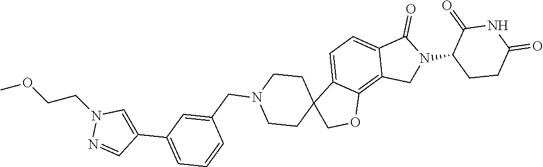 [SMILES: COCCn1cc(-c2cccc(CN3CCC4(CC3)COc3c4ccc4c3CN([C@H]3CCC(=O)NC3=O)C4=O)c2)cn1] | (S)-3-(1′-(3-(1-(2-methoxyethyl)-1H- pyrazol-4-yl)benzyl)-6-oxo-6,8- dihydro- 2H,7H-spiro[furo[2,3-e]isoindole-3,4′- piperidin]-7-yl)piperidine-2,6-dione |
| A287 | 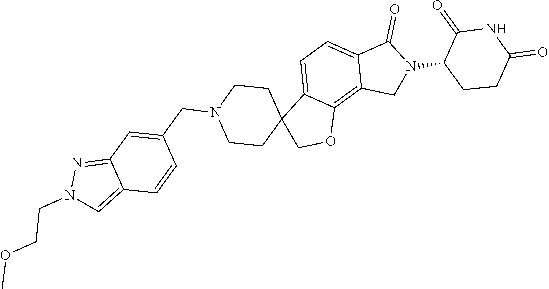 [SMILES: COCCn1cc2ccc(CN3CCC4(CC3)COc3c4ccc4c3CN([C@H]3CCC(=O)NC3=O)C4=O)cc2n1] | (S)-3-(1′-((2-(2-methoxyethyl)-2H- indazol-6-yl)methyl)-6-oxo-6,8- dihydro- 2H,7H-spiro[furo[2,3-e]isoindole-3,4′- piperidin]-7-yl)piperidine-2,6-dione |
| A288 | 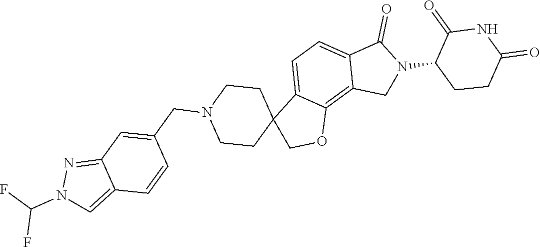 [SMILES: O=C1CC[C@H](N2Cc3c(ccc4c3OCC43CCN(Cc4ccc5cn(C(F)F)nc5c4)CC3)C2=O)C(=O)N1] | (S)-3-(1′-((2-(difluoromethyl)-2H- indazol-6-yl)methyl)-6-oxo-6,8- dihydro- 2H,7H-spiro[furo[2,3-e]isoindole-3,4′- piperidin]-7-yl)piperidine-2,6-dione |
| A289 | 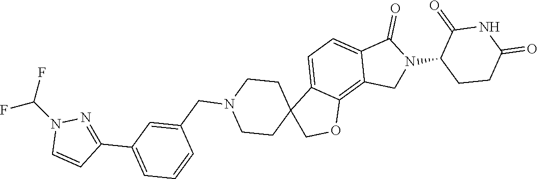 [SMILES: O=C1CC[C@H](N2Cc3c(ccc4c3OCC43CCN(Cc4cccc(-c5ccn(C(F)F)n5)c4)CC3)C2=O)C(=O)N1] | (S)-3-(1′-(3-(1-(difluoromethyl)-1H- pyrazol-3-yl)benzyl)-6-oxo-6,8- dihydro- 2H,7H-spiro[furo[2,3-e]isoindole-3,4′- piperidin]-7-yl)piperidine-2,6-dione |
| A290 | 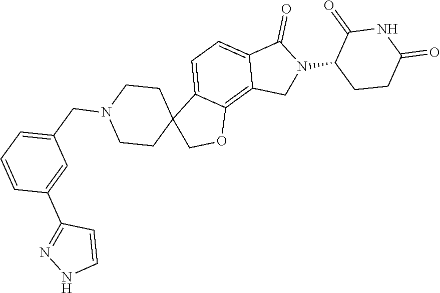 [SMILES: O=C1CC[C@H](N2Cc3c(ccc4c3OCC43CCN(Cc4cccc(-c5cc[nH]n5)c4)CC3)C2=O)C(=O)N1] | (S)-3-(1′-(3-(1H-pyrazol-3-yl)benzyl)- 6-oxo-6,8-dihydro- 2H,7H-spiro[furo[2,3-e]isoindole-3,4′- piperidin]-7-yl)piperidine-2,6-dione |
| A291 | 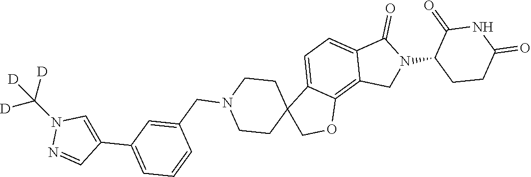 [SMILES: [2H]C([2H])([2H])n1cc(-c2cccc(CN3CCC4(CC3)COc3c4ccc4c3CN([C@H]3CCC(=O)NC3=O)C4=O)c2)cn1] | (S)-3-(1′-(3-(1-(methyl-d3)-1H- pyrazol-4-yl)benzyl)-6-oxo-6,8- dihydro- 2H,7H-spiro[furo[2,3-e]isoindole-3,4′- piperidin]-7-yl)piperidine-2,6-dione |
| A292 | 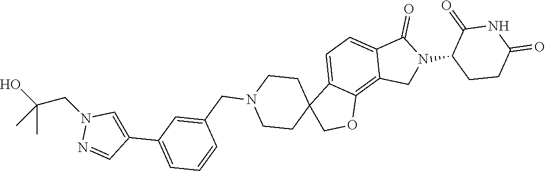 [SMILES: CC(C)(O)Cn1cc(-c2cccc(CN3CCC4(CC3)COc3c4ccc4c3CN([C@H]3CCC(=O)NC3=O)C4=O)c2)cn1] | (S)-3-(1′-(3-(1-(2-hydroxy-2- methylpropyl)-1H-pyrazol-4- yl)benzyl)-6-oxo-6,8-dihydro- 2H,7H-spiro[furo[2,3-e]isoindole-3,4′- piperidin]-7-yl)piperidine-2,6-dione |
| A293 | 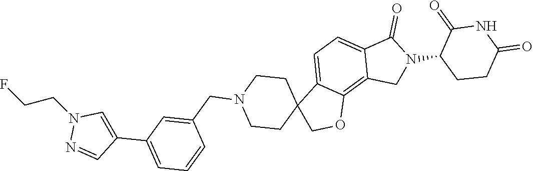 [SMILES: O=C1CC[C@H](N2Cc3c(ccc4c3OCC43CCN(Cc4cccc(-c5cnn(CCF)c5)c4)CC3)C2=O)C(=O)N1] | (S)-3-(1′-(3-(1-(2-fluoroethyl)-1H- pyrazol-4-yl)benzyl)-6-oxo-6,8- dihydro- 2H,7H-spiro[furo[2,3-e]isoindole-3,4′- piperidin]-7-yl)piperidine-2,6-dione |
| A294 | 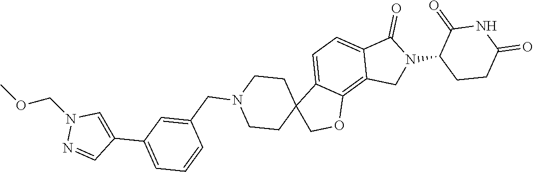 [SMILES: COCn1cc(-c2cccc(CN3CCC4(CC3)COc3c4ccc4c3CN([C@H]3CCC(=O)NC3=O)C4=O)c2)cn1] | (S)-3-(1′-(3-(1-(methoxymethyl)-1H- pyrazol-4-yl)benzyl)-6-oxo-6,8- dihydro- 2H,7H-spiro[furo[2,3-e]isoindole-3,4′- piperidin]-7-yl)piperidine-2,6-dione |
| A295 | 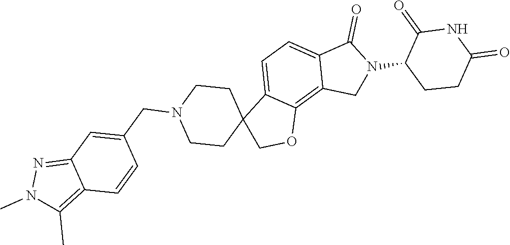 [SMILES: Cc1c2ccc(CN3CCC4(CC3)COc3c4ccc4c3CN([C@H]3CCC(=O)NC3=O)C4=O)cc2nn1C] | (S)-3-(1′-((2,3-dimethyl-2H-indazol- 6-yl)methyl)-6-oxo-6,8-dihydro- 2H,7H-spiro[furo[2,3-e]isoindole-3,4′- piperidin]-7-yl)piperidine-2,6-dione |
| A296 | 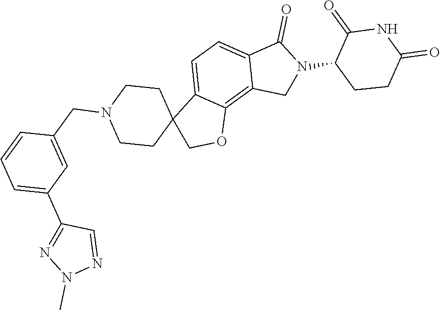 [SMILES: Cn1ncc(-c2cccc(CN3CCC4(CC3)COc3c4ccc4c3CN([C@H]3CCC(=O)NC3=O)C4=O)c2)n1] | (S)-3-(1′-(3-(2-methyl-2H-1,2,3- triazol-4-yl)benzyl)-6-oxo-6,8- dihydro- 2H,7H-spiro[furo[2,3-e]isoindole-3,4′- piperidin]-7-yl)piperidine-2,6-dione |
| A297 | 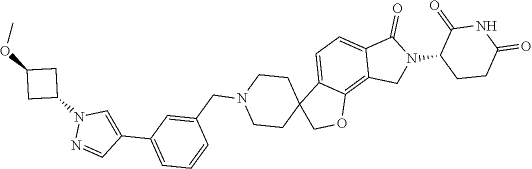 [SMILES: CO[C@H]1C[C@H](n2cc(-c3cccc(CN4CCC5(CC4)COc4c5ccc5c4CN([C@H]4CCC(=O)NC4=O)C5=O)c3)cn2)C1] | (S)-3-(1′-(3-(1-((1r,3r)-3- methoxycyclobutyl)-1H-pyrazol-4- yl)benzyl)-6-oxo-6,8-dihydro- 2H,7H-spiro[furo[2,3-e]isoindole-3,4′- piperidin]-7-yl)piperidine-2,6-dione |
| A298 | 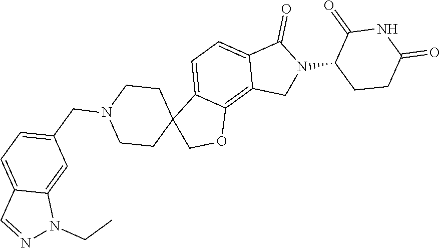 [SMILES: CCn1ncc2ccc(CN3CCC4(CC3)COc3c4ccc4c3CN([C@H]3CCC(=O)NC3=O)C4=O)cc21] | (S)-3-(1′-((1-ethyl-1H-indazol-6- yl)methyl)-6-oxo-6,8-dihydro- 2H,7H-spiro[furo[2,3-e]isoindole-3,4′- piperidin]-7-yl)piperidine-2,6-dione |
| A299 | 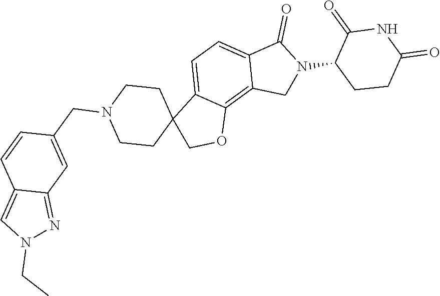 [SMILES: CCn1cc2ccc(CN3CCC4(CC3)COc3c4ccc4c3CN([C@H]3CCC(=O)NC3=O)C4=O)cc2n1] | (S)-3-(1′-((2-ethyl-2H-indazol-6- yl)methyl)-6-oxo-6,8-dihydro- 2H,7H-spiro[furo[2,3-e]isoindole-3,4′- piperidin]-7-yl)piperidine-2,6-dione |
| A300 | 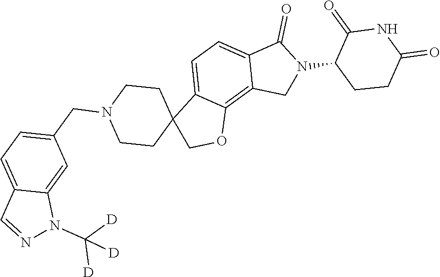 [SMILES: [2H]C([2H])([2H])n1ncc2ccc(CN3CCC4(CC3)COc3c4ccc4c3CN([C@H]3CCC(=O)NC3=O)C4=O)cc21] | (S)-3-(1′-((1-(methyl-d3)-1H-indazol- 6-yl)methyl)-6-oxo-6,8-dihydro- 2H,7H-spiro[furo[2,3-e]isoindole-3,4′- piperidin]-7-yl)piperidine-2,6-dione |
| A301 | 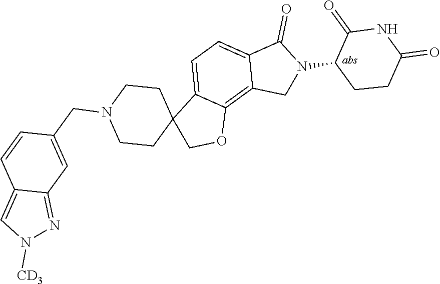 [SMILES: [2H]C([2H])([2H])n1cc2ccc(CN3CCC4(CC3)COc3c4ccc4c3CN([C@H]3CCC(=O)NC3=O)C4=O)cc2n1] | (S)-3-(1′-((2-(methyl-d3)-2H-indazol- 6-yl)methyl)-6-oxo-6,8-dihydro- 2H,7H-spiro[furo[2,3-e]isoindole-3,4′- piperidin]-7-yl)piperidine-2,6-dione |
| A302 | 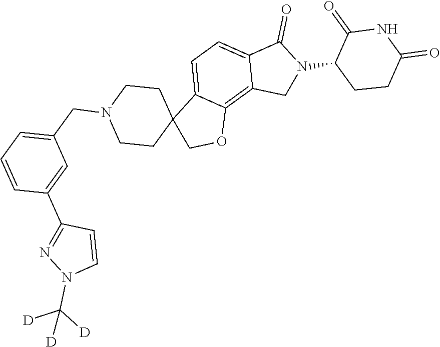 [SMILES: [2H]C([2H])([2H])n1ccc(-c2cccc(CN3CCC4(CC3)COc3c4ccc4c3CN([C@H]3CCC(=O)NC3=O)C4=O)c2)n1] | (S)-3-(1′-(3-(1-(methyl-d3)-1H- pyrazol-3-yl)benzyl)-6-oxo-6,8- dihydro- 2H,7H-spiro[furo[2,3-e]isoindole-3,4′- piperidin]-7-yl)piperidine-2,6-dione |
| A303 | 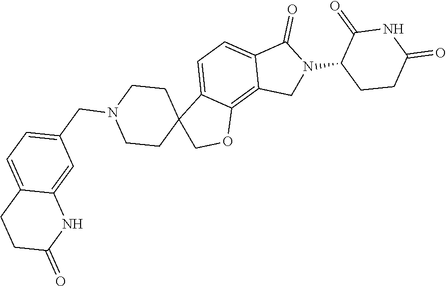 [SMILES: O=C1CC[C@H](N2Cc3c(ccc4c3OCC43CCN(Cc4ccc5c(c4)NC(=O)CC5)CC3)C2=O)C(=O)N1] | 3-(6-oxo-1′-((2-oxo-1,2,3,4- tetrahydroquinolin-7-yl)methyl)-6,8- dihydro- 2H,7H-spiro[furo[2,3-e]isoindole-3,4′- piperidin]-7-yl)piperidine-2,6-dione |
| A304 | 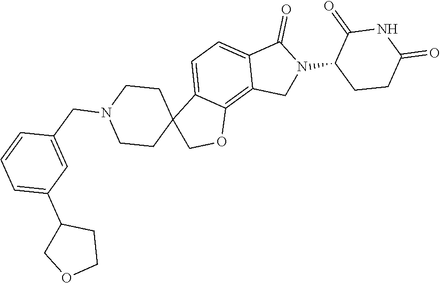 [SMILES: O=C1CC[C@H](N2Cc3c(ccc4c3OCC43CCN(Cc4cccc(C5CCOC5)c4)CC3)C2=O)C(=O)N1] | (3S)-3-(6-oxo-1′-(3-(tetrahydrofuran- 3-yl)benzyl)-6,8-dihydro- 2H,7H-spiro[furo[2,3-e]isoindole-3,4′- piperidin]-7-yl)piperidine-2,6-dione |
| A305 | 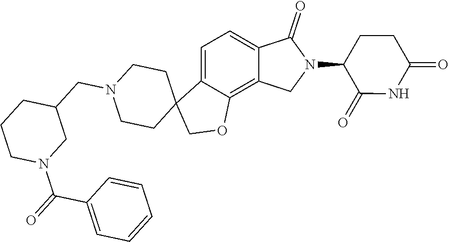 [SMILES: O=C1CC[C@H](N2Cc3c(ccc4c3OCC43CCN(CC4CCCN(C(=O)c5ccccc5)C4)CC3)C2=O)C(=O)N1] | (3S)-3-(1′-((1-benzoylpiperidin-3- yl)methyl)-6-oxo-6,8-dihydro- 2H,7H-spiro[furo[2,3-e]isoindole-3,4′- piperidin]-7-yl)piperidine-2,6-dione |
| A306 | 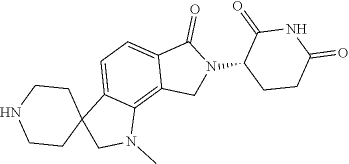 [SMILES: CN1CC2(CCNCC2)c2ccc3c(c21)CN([C@H]1CCC(=O)NC1=O)C3=O] | (S)-3-(1′-methyl-6′-oxo-1′,2′,6′,8′- tetrahydro-7′H-spiro[piperidine-4,3′- pyrrolo[3,4-g]indol]-7′-yl)piperidine- 2,6-dione |
| A307 | 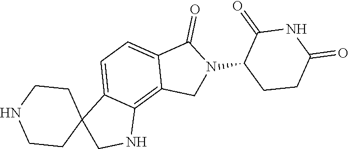 [SMILES: O=C1CC[C@H](N2Cc3c(ccc4c3NCC43CCNCC3)C2=O)C(=O)N1] | (S)-3-(6′-oxo-1′,2′,6′,8′-tetrahydro- 7′H-spiro[piperidine-4,3′-pyrrolo[3,4- g]indol-7′-yl)piperidine-2,6-dione |
| A308 | 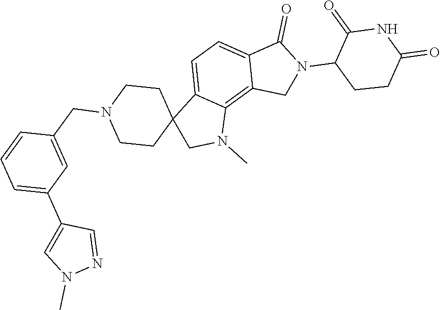 [SMILES: CN1CC2(CCN(Cc3cccc(-c4cnn(C)c4)c3)CC2)c2ccc3c(c21)CN(C1CCC(=O)NC1=O)C3=O] | 3-(1′-methyl-1-(3-(1-methyl-1H- pyrazol-4-yl)benzyl)-6′-oxo- 1′,2′,6′,8′-tetrahydro-7′H- spiro[piperidine-4,3′-pyrrolo[3,4- g]indol]-7′-yl)piperidine-2,6-dione |
| A309 | 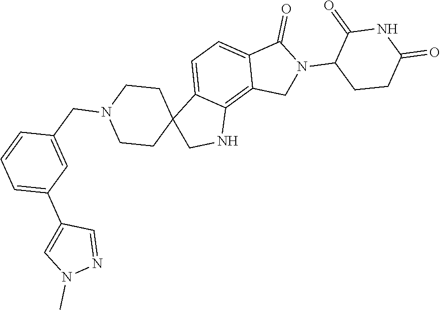 [SMILES: Cn1cc(-c2cccc(CN3CCC4(CC3)CNc3c4ccc4c3CN(C3CCC(=O)NC3=O)C4=O)c2)cn1] | 3-(1-(3-(1-methyl-1H-pyrazol-4- yl)benzyl)-6′-oxo-1′,2′,6′,8′- tetrahydro-7′H-spiro[piperidine-4,3′- pyrrolo[3,4-g]indol]-7′- yl)piperidine-2,6-dione |
| A310 | 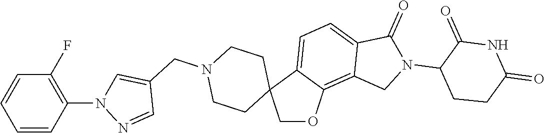 [SMILES: O=C1CCC(N2Cc3c(ccc4c3OCC43CCN(Cc4cnn(-c5ccccc5F)c4)CC3)C2=O)C(=O)N1] | 3-(1′-((1-(2-fluorophenyl)-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A311 | 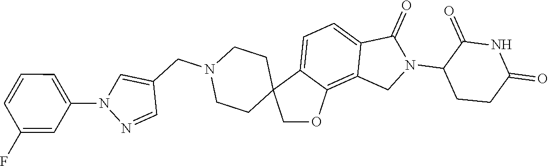 [SMILES: O=C1CCC(N2Cc3c(ccc4c3OCC43CCN(Cc4cnn(-c5cccc(F)c5)c4)CC3)C2=O)C(=O)N1] | 3-(1′-((1-(3-fluorophenyl)-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A312 | 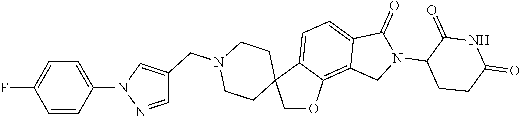 [SMILES: O=C1CCC(N2Cc3c(ccc4c3OCC43CCN(Cc4cnn(-c5ccc(F)cc5)c4)CC3)C2=O)C(=O)N1] | 3-(1′-((1-(4-fluorophenyl)-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A313 | 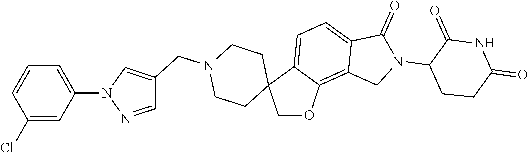 [SMILES: O=C1CCC(N2Cc3c(ccc4c3OCC43CCN(Cc4cnn(-c5cccc(Cl)c5)c4)CC3)C2=O)C(=O)N1] | 3-(1′-((1-(3-chlorophenyl)-1H- pyrazol-4-yl)methyl)-6-oxo-6,8- dihydro- 2H,7H-spiro[furo[2,3-e]isoindole-3,4′- piperidin]-7-yl)piperidine-2,6-dione |
| A314 | 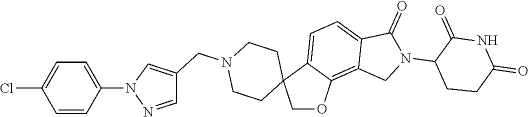 [SMILES: O=C1CCC(N2Cc3c(ccc4c3OCC43CCN(Cc4cnn(-c5ccc(Cl)cc5)c4)CC3)C2=O)C(=O)N1] | 3-(1′-((1-(4-chlorophenyl)-1H- pyrazol-4-yl)methyl)-6-oxo-6,8- dihydro- 2H,7H-spiro[furo[2,3-e]isoindole-3,4′- piperidin]-7-yl)piperidine-2,6-dione |
| A315 | 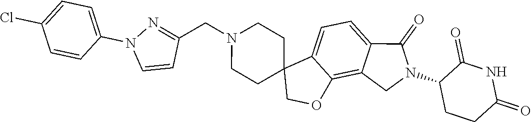 [SMILES: O=C1CC[C@H](N2Cc3c(ccc4c3OCC43CCN(Cc4ccn(-c5ccc(Cl)cc5)n4)CC3)C2=O)C(=O)N1] | (S)-3-(1′-((1-(4-chlorophenyl)-1H- pyrazol-3-yl)methyl)-6-oxo-6,8- dihydro- 2H,7H-spiro[furo[2,3-e]isoindole-3,4′- piperidin]-7-yl)piperidine-2,6-dione |
| A316 | 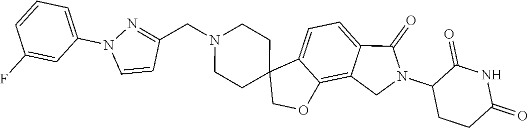 [SMILES: O=C1CCC(N2Cc3c(ccc4c3OCC43CCN(Cc4ccn(-c5cccc(F)c5)n4)CC3)C2=O)C(=O)N1] | 3-(1′-((1-(3-fluorophenyl)-1H-pyrazol- 3-yl)methyl)-6-oxo-6,8-dihydro- 2H,7H-spiro[furo[2,3-e]isoindole-3,4′- piperidin]-7-yl)piperidine-2,6-dione |
| A317 | 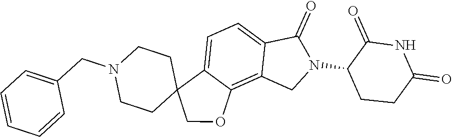 [SMILES: O=C1CC[C@H](N2Cc3c(ccc4c3OCC43CCN(Cc4ccccc4)CC3)C2=O)C(=O)N1] | (S)-3-(1′-benzyl-6-oxo-6,8-dihydro- 2H,7H-spiro[furo[2,3-e]isoindole-3,4′- piperidin]-7-yl)piperidine-2,6-dione |
| A318 | 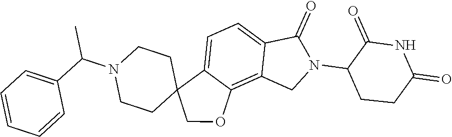 [SMILES: CC(c1ccccc1)N1CCC2(CC1)COc1c2ccc2c1CN(C1CCC(=O)NC1=O)C2=O] | 3-(6-oxo-1′-(1-phenylethyl)-6,8- dihydro-2H,7H-spiro[furo [2,3-e]isoindole-3,4′- piperidin]-7-yl)piperidine-2,6-dione |
| A319 | 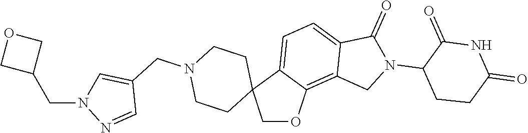 [SMILES: O=C1CCC(N2Cc3c(ccc4c3OCC43CCN(Cc4cnn(CC5COC5)c4)CC3)C2=O)C(=O)N1] | 3-(1′-((1-(oxetan-3-ylmethyl)-1H- pyrazol-4-yl)methyl)-6-oxo-6,8- dihydro- 2H,7H-spiro[furo[2,3-e]isoindole-3,4′- piperidin]-7-yl)piperidine-2,6-dione |
| A320 | 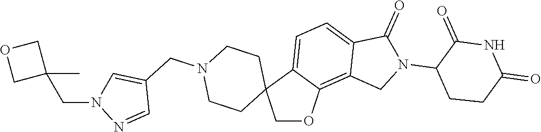 [SMILES: CC1(Cn2cc(CN3CCC4(CC3)COc3c4ccc4c3CN(C3CCC(=O)NC3=O)C4=O)cn2)COC1] | 3-(1′-((1-((3-methyloxetan-3- yl)methyl)-1H-pyrazol-4-yl)methyl)- 6-oxo-6,8-dihydro- 2H,7H-spiro[furo[2,3-e]isoindole-3,4′- piperidin]-7-yl)piperidine-2,6-dione |
| A321 | 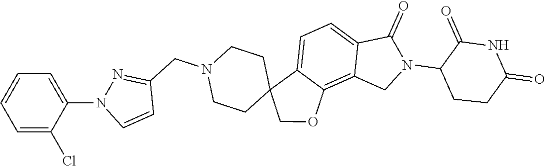 [SMILES: O=C1CCC(N2Cc3c(ccc4c3OCC43CCN(Cc4ccn(-c5ccccc5Cl)n4)CC3)C2=O)C(=O)N1] | 3-(1′-((1-(2-chlorophenyl)-1H- pyrazol-3-yl)methyl)-6-oxo-6,8- dihydro- 2H,7H-spiro[furo[2,3-e]isoindole-3,4′- piperidin]-7-yl)piperidine-2,6-dione |
| A322 | 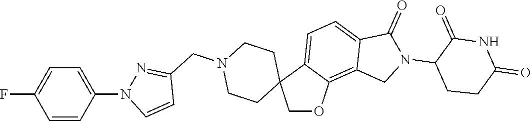 [SMILES: O=C1CCC(N2Cc3c(ccc4c3OCC43CCN(Cc4ccn(-c5ccc(F)cc5)n4)CC3)C2=O)C(=O)N1] | 3-(1′-((1-(4-fluorophenyl)-1H-pyrazol- 3-yl)methyl)-6-oxo-6,8-dihydro- 2H,7H-spiro[furo[2,3-e]isoindole-3,4′- piperidin]-7-yl)piperidine-2,6-dione |
| A323 | 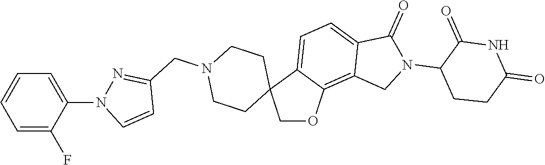 [SMILES: O=C1CCC(N2Cc3c(ccc4c3OCC43CCN(Cc4ccn(-c5ccccc5F)n4)CC3)C2=O)C(=O)N1] | 3-(1′-((1-(2-fluorophenyl)-1H-pyrazol- 3-yl)methyl)-6-oxo-6,8-dihydro- 2H,7H-spiro[furo[2,3-e]isoindole-3,4′- piperidin]-7-yl)piperidine-2,6-dione |
| A324 | 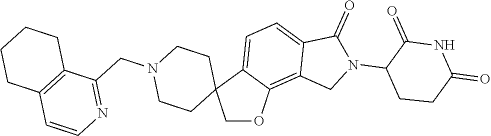 [SMILES: O=C1CCC(N2Cc3c(ccc4c3OCC43CCN(Cc4nccc5c4CCCC5)CC3)C2=O)C(=O)N1] | 3-(6-oxo-1′-((5,6,7,8- tetrahydroisoquinolin-1-yl)methyl)- 6,8-dihydro-2H,7H-spiro[furo [2,3-e]isoindole-3,4′-piperidin]- 7-yl)piperidine-2,6-dione |
| A325 | 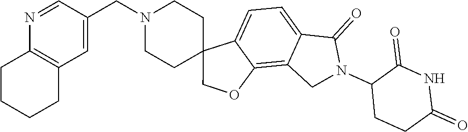 [SMILES: O=C1CCC(N2Cc3c(ccc4c3OCC43CCN(Cc4cnc5c(c4)CCCC5)CC3)C2=O)C(=O)N1] | 3-(6-oxo-1′-((5,6,7,8- tetrahydroquinolin-3-yl)methyl)-6,8- dihydro-2H,7H-spiro[furo [2,3-e]isoindole-3,4′-piperidin]- 7-yl)piperidine-2,6-dione |
| A326 | 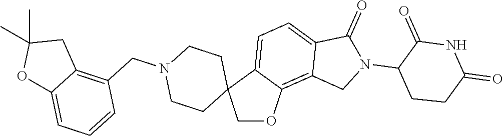 [SMILES: CC1(C)Cc2c(CN3CCC4(CC3)COc3c4ccc4c3CN(C3CCC(=O)NC3=O)C4=O)cccc2O1] | 3-(1′-((2,2-dimethyl-2,3- dihydrobenzofuran-4-yl)methyl)-6- oxo-6,8-dihydro- 2H,7H-spiro[furo[2,3-e]isoindole-3,4′- piperidin]-7-yl)piperidine-2,6-dione |
| A327 | 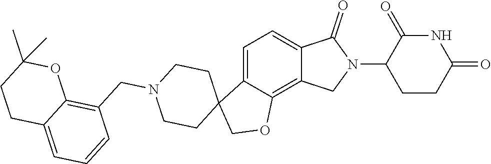 [SMILES: CC1(C)CCc2cccc(CN3CCC4(CC3)COc3c4ccc4c3CN(C3CCC(=O)NC3=O)C4=O)c2O1] | 3-(1′-((2,2-dimethylchroman-8- yl)methyl)-6-oxo-6,8-dihydro- 2H,7H-spiro[furo[2,3-e]isoindole-3,4′- piperidin]-7-yl)piperidine-2,6-dione |
| A328 | 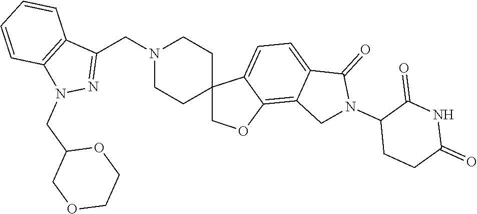 [SMILES: O=C1CCC(N2Cc3c(ccc4c3OCC43CCN(Cc4nn(CC5COCCO5)c5ccccc45)CC3)C2=O)C(=O)N1] | 3-(1′-((1-((1,4-dioxan-2-yl)methyl)- 1H-indazol-3-yl)methyl)-6-oxo-6,8- dihydro- 2H,7H-spiro[furo[2,3-e]isoindole-3,4′- piperidin]-7-yl)piperidine-2,6-dione |
| A329 | 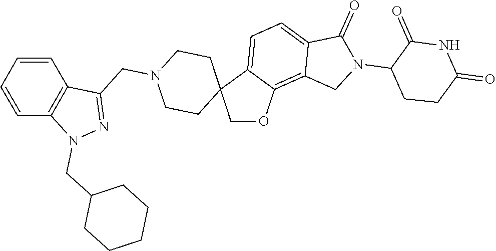 [SMILES: O=C1CCC(N2Cc3c(ccc4c3OCC43CCN(Cc4nn(CC5CCCCC5)c5ccccc45)CC3)C2=O)C(=O)N1] | 3-(1′-((1-(cyclohexylmethyl)-1H- indazol-3-yl)methyl)-6-oxo-6,8- 2H,7H-spiro[furo[2,3-e]isoindole-3,4′- piperidin]-7-yl)piperidine-2,6-dione |
| A330 | 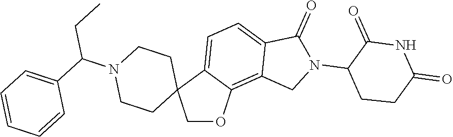 [SMILES: CCC(c1ccccc1)N1CCC2(CC1)COc1c2ccc2c1CN(C1CCC(=O)NC1=O)C2=O] | 3-(6-oxo-1′-(1-phenylpropyl)-6,8- dihydro- 2H,7H-spiro[furo[2,3-e]isoindole-3,4′- piperidin]-7-yl)piperidine-2,6-dione |
| A331 | 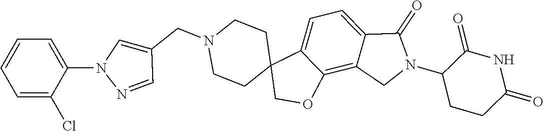 [SMILES: O=C1CCC(N2Cc3c(ccc4c3OCC43CCN(Cc4cnn(-c5ccccc5Cl)c4)CC3)C2=O)C(=O)N1] | 3-(1′-((1-(2-chlorophenyl)-1H- pyrazol-4-yl)methyl)-6-oxo-6,8- dihydro-2H,7H-spiro[furo[2,3- e]isoindole-3,4′-piperidin]-7-yl) piperidine-2,6-dione |
| A332 | 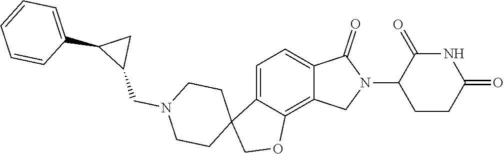 [SMILES: O=C1CCC(N2Cc3c(ccc4c3OCC43CCN(C[C@H]4C[C@@H]4c4ccccc4)CC3)C2=O)C(=O)N1] | 3-(6-oxo-1′-(((1S,2S)-2- phenylcyclopropyl)methyl)-6,8- dihydro- 2H,7H-spiro[furo[2,3-e]isoindole-3,4′- piperidin]-7-yl)piperidine-2,6-dione |
| A333 | 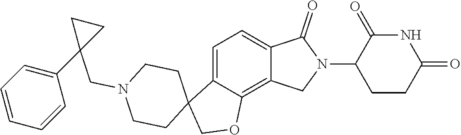 [SMILES: O=C1CCC(N2Cc3c(ccc4c3OCC43CCN(CC4(c5ccccc5)CC4)CC3)C2=O)C(=O)N1] | 3-(6-oxo-1′-((1- phenylcyclopropyl)methyl)-6,8- dihydro- 2H,7H-spiro[furo[2,3-e]isoindole-3,4′- piperidin]-7-yl)piperidine-2,6-dione |
| A334 | 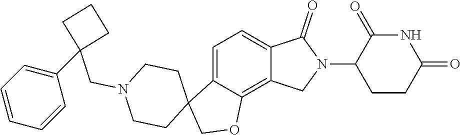 [SMILES: O=C1CCC(N2Cc3c(ccc4c3OCC43CCN(CC4(c5ccccc5)CCC4)CC3)C2=O)C(=O)N1] | 3-(6-oxo-1′-((1- phenylcyclobutyl)methyl)-6,8- dihydro- 2H,7H-spiro[furo[2,3-e]isoindole-3,4′- piperidin]-7-yl)piperidine-2,6-dione |
| A335 | 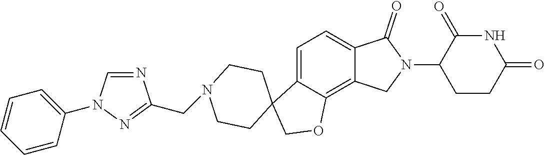 [SMILES: O=C1CCC(N2Cc3c(ccc4c3OCC43CCN(Cc4ncn(-c5ccccc5)n4)CC3)C2=O)C(=O)N1] | 3-(6-oxo-1′-((1-phenyl-1H-1,2,4- triazol-3-yl)methyl)-6,8-dihydro- 2H,7H-spiro[furo[2,3-e]isoindole-3,4′- piperidin]-7-yl)piperidine-2,6-dione |
| A336 | 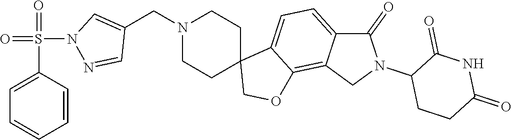 [SMILES: O=C1CCC(N2Cc3c(ccc4c3OCC43CCN(Cc4cnn(S(=O)(=O)c5ccccc5)c4)CC3)C2=O)C(=O)N1] | 3-(6-oxo-1′-((1-(phenylsulfonyl)-1H- pyrazol-4-yl)methyl)-6,8-dihydro- 2H,7H-spiro[furo[2,3-e]isoindole- 3,4′-piperidin]-7-yl)piperidine-2,6- dione |
| A337 | 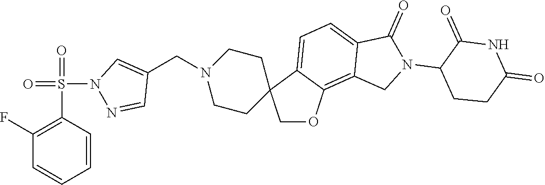 [SMILES: O=C1CCC(N2Cc3c(ccc4c3OCC43CCN(Cc4cnn(S(=O)(=O)c5ccccc5F)c4)CC3)C2=O)C(=O)N1] | 3-(1′-((1-((2-fluorophenyl)sulfonyl)- 1H-pyrazol-4-yl)methyl)-6-oxo-6,8- dihydro-2H,7H-spiro[furo[2,3- e]isoindole-3,4′-piperidin]-7- yl)piperidine-2,6-dione |
| A338 | 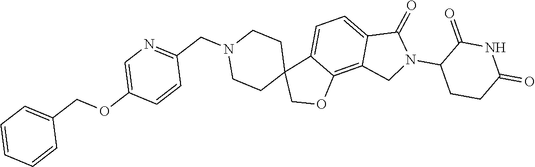 [SMILES: O=C1CCC(N2Cc3c(ccc4c3OCC43CCN(Cc4ccc(OCc5ccccc5)cn4)CC3)C2=O)C(=O)N1] | 3-(1′-((5-(benzyloxy)pyridin-2- yl)methyl)-6-oxo-6,8-dihydro- 2H,7H-spiro[furo[2,3-e]isoindole-3,4′- piperidin]-7-yl)piperidine-2,6-dione |
| A339 | 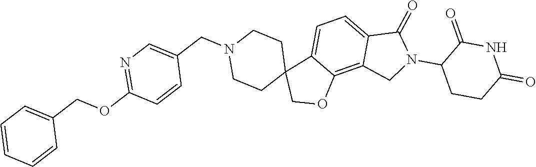 [SMILES: O=C1CCC(N2Cc3c(ccc4c3OCC43CCN(Cc4ccc(OCc5ccccc5)nc4)CC3)C2=O)C(=O)N1] | 3-(1′-((6-(benzyloxy)pyridin-3- yl)methyl)-6-oxo-6,8-dihydro-2H,7H- spiro[furo[2,3-e]isoindole-3,4′- piperidin]-7-yl)piperidine-2,6-dione |
| A340 | 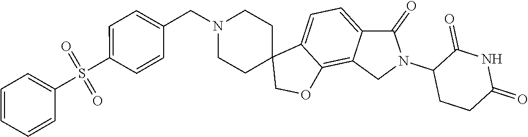 [SMILES: O=C1CCC(N2Cc3c(ccc4c3OCC43CCN(Cc4ccc(S(=O)(=O)c5ccccc5)cc4)CC3)C2=O)C(=O)N1] | 3-(6-oxo-1′-(4- (phenylsulfonyl)benzyl)-6,8-dihydro- 2H,7H-spiro[furo[2,3-e]isoindole-3,4′- piperidin]-7-yl)piperidine-2,6-dione |
| A341 | 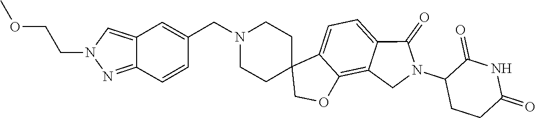 [SMILES: COCCn1cc2cc(CN3CCC4(CC3)COc3c4ccc4c3CN(C3CCC(=O)NC3=O)C4=O)ccc2n1] | 3-(1′-((2-(2-methoxyethyl)-2H- indazol-5-yl)methyl)-6-oxo-6,8- dihydro-2H,7H-spiro[furo[2,3-e] isoindole-3,4′-piperidin]-7-yl) piperidine-2,6-dione |
| A342 | 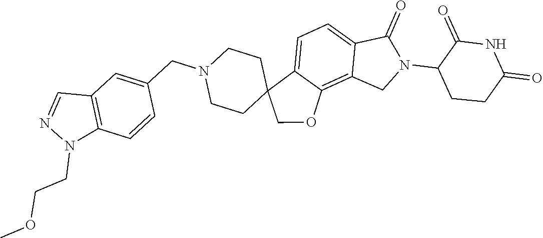 [SMILES: COCCn1ncc2cc(CN3CCC4(CC3)COc3c4ccc4c3CN(C3CCC(=O)NC3=O)C4=O)ccc21] | 3-(1′-((1-(2-methoxyethyl)-1H- indazol-5-yl)methyl)-6-oxo-6,8- dihydro- 2H,7H-spiro[furo[2,3-e]isoindole-3,4′- piperidin]-7-yl)piperidine-2,6-dione |
| A343 | 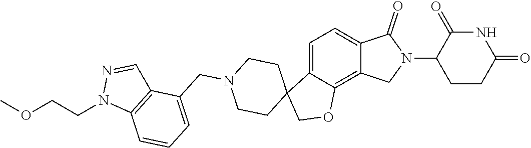 [SMILES: COCCn1ncc2c(CN3CCC4(CC3)COc3c4ccc4c3CN(C3CCC(=O)NC3=O)C4=O)cccc21] | 3-(1′-((1-(2-methoxyethyl)-1H- indazol-4-yl)methyl)-6-oxo-6,8- dihydro- 2H,7H-spiro[furo[2,3-e]isoindole-3,4′- piperidin]-7-yl)piperidine-2,6-dione |
| A344 | 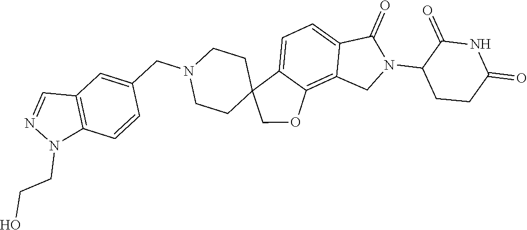 [SMILES: O=C1CCC(N2Cc3c(ccc4c3OCC43CCN(Cc4ccc5c(cnn5CCO)c4)CC3)C2=O)C(=O)N1] | 3-(1′-((1-(2-hydroxyethyl)-1H- indazol-5-yl)methyl)-6-oxo-6,8- dihydro- 2H,7H-spiro[furo[2,3-e]isoindole-3,4′- piperidin]-7-yl)piperidine-2,6-dione |
| A345 | 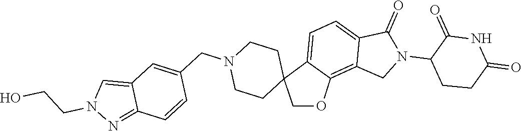 [SMILES: O=C1CCC(N2Cc3c(ccc4c3OCC43CCN(Cc4ccc5nn(CCO)cc5c4)CC3)C2=O)C(=O)N1] | 3-(1′-((2-(2-hydroxyethyl)-2H- indazol-5-yl)methyl)-6-oxo-6,8- dihydro-2H,7H-spiro [furo[2,3-e]isoindole-3,4′- piperidin]-7-yl)piperidine-2,6-dione |
| A346 | 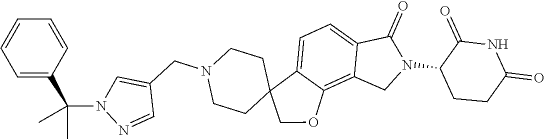 | (S)-3-(6-oxo-1′-((1-(2-phenylpropan- 2-yl)-1H-pyrazol-4-yl)methyl)-6,8- dihydro- 2H,7H-spiro[furo[2,3-e]isoindole-3,4′- piperidin]-7-yl)piperidine-2,6-dione |
| A347 | 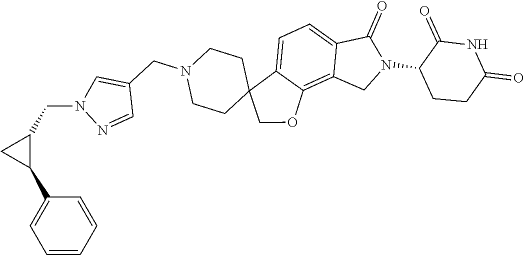 | (S)-3-(6-oxo-1′-((1-(((1S,2S)-2- phenylcyclopropyl)methyl)-1H- pyrazol-4-yl)methyl)-6,8-dihydro- 2H,7H-spiro[furo[2,3-e]isoindole-3,4′- piperidin]-7-yl)piperidine-2,6-dione |
| A348 | 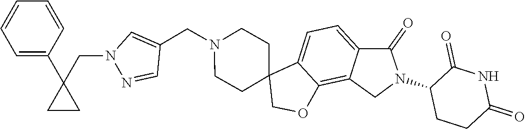 [SMILES: O=C1CC[C@H](N2Cc3c(ccc4c3OCC43CCN(Cc4cnn(CC5(c6ccccc6)CC5)c4)CC3)C2=O)C(=O)N1] | (S)-3-(6-oxo-1′-((1-((1- phenylcyclopropyl)methyl)-1H- pyrazol-4-yl)methyl)-6,8-dihydro- 2H,7H-spiro[furo[2,3-e]isoindole-3,4′- piperidin]-7-yl)piperidine-2,6-dione |
| A349 | 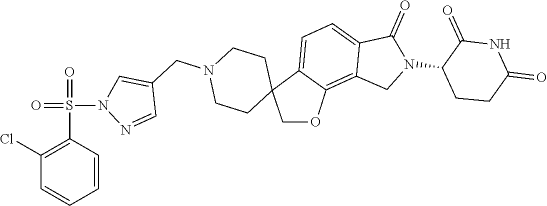 [SMILES: O=C1CC[C@H](N2Cc3c(ccc4c3OCC43CCN(Cc4cnn(S(=O)(=O)c5ccccc5Cl)c4)CC3)C2=O)C(=O)N1] | (S)-3-(1′-((1-((2- chlorophenyl)sulfonyl)-1H-pyrazol-4- yl)methyl)-6-oxo-6,8-dihydro- 2H,7H-spiro[furo[2,3-e]isoindole-3,4′- piperidin]-7-yl)piperidine-2,6-dione |
| A350 | 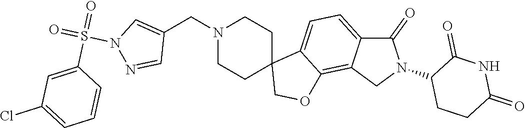 | (S)-3-(1′-((1-((3- chlorophenyl)sulfonyl)-1H-pyrazol-4- yl)methyl)-6-oxo-6,8-dihydro- 2H,7H-spiro[furo[2,3-e]isoindole-3,4′- piperidin]-7-yl)piperidine-2,6-dione |
| A351 | 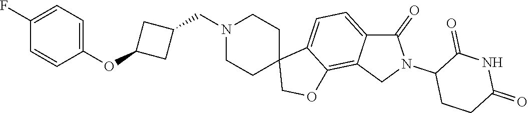 [SMILES: O=C1CCC(N2Cc3c(ccc4c3OCC43CCN(C[C@H]4C[C@H](Oc5ccc(F)cc5)C4)CC3)C2=O)C(=O)N1] | 3-(1′-(((1r,3r)-3-(4- fluorophenoxy)cyclobutyl)methyl)-6- oxo-6,8-dihydro- 2H,7H-spiro[furo[2,3-e]isoindole-3,4′- piperidin]-7-yl)piperidine-2,6-dione |
| A352 | 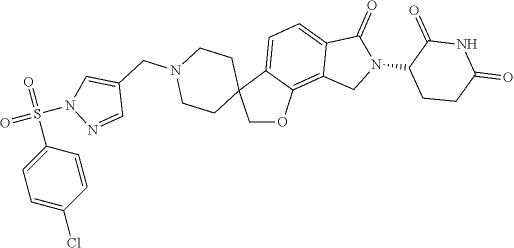 | (S)-3-(1′-((1-((4- chlorophenyl)sulfonyl)-1H-pyrazol-4- yl)methyl)-6-oxo-6,8-dihydro- 2H,7H-spiro[furo[2,3-e]isoindole-3,4′- piperidin]-7-yl)piperidine-2,6-dione |
| A353 | 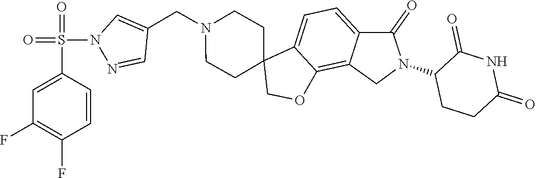 | (S)-3-(1′-((1-((3,4- difluorophenyl)sulfonyl)-1H-pyrazol- 4-yl)methyl)-6-oxo-6,8-dihydro- 2H,7H-spiro[furo[2,3-e]isoindole-3,4′- piperidin]-7-yl)piperidine-2,6-dione |
| A354 | 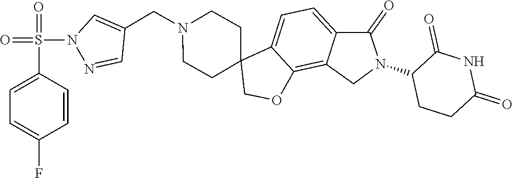 | (S)-3-(1′-((1-((4- fluorophenyl)sulfonyl)-1H-pyrazol-4- yl)methyl)-6-oxo-6,8-dihydro- 2H,7H-spiro[furo[2,3-e]isoindole-3,4′- piperidin]-7-yl)piperidine-2,6-dione |
| A355 | 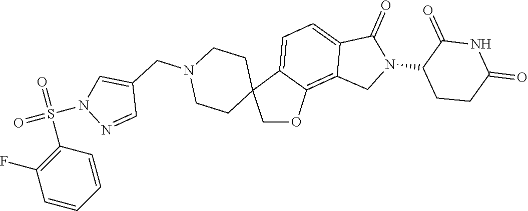 | (S)-3-(1′-((1-((2- fluorophenyl)sulfonyl)-1H-pyrazol-4- yl)methyl)-6-oxo-6,8-dihydro- 2H,7H-spiro[furo[2,3-e]isoindole-3,4′- piperidin]-7-yl)piperidine-2,6-dione |
| A356 | 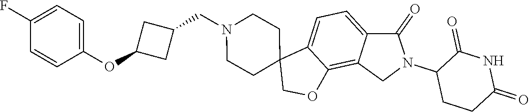 [SMILES: O=C1CCC(N2Cc3c(ccc4c3OCC43CCN(C[C@H]4C[C@H](Oc5ccc(F)cc5)C4)CC3)C2=O)C(=O)N1] | 3-(1′-(((1r,3r)-3-(4- fluorophenoxy)cyclobutyl)methyl)-6- oxo-6,8-dihydro- 2H,7H-spiro[furo[2,3-e]isoindole-3,4′- piperidin]-7-yl)piperidine-2,6-dione |
| A357 | 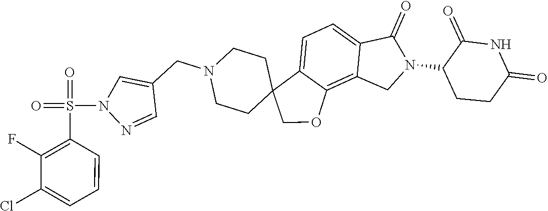 | (S)-3-(1′-((1-((3-chloro-2- fluorophenyl)sulfonyl)-1H-pyrazol-4- yl)methyl)-6-oxo-6,8-dihydro- 2H,7H-spiro[furo[2,3-e]isoindole-3,4′- piperidin]-7-yl)piperidine-2,6-dione |
| A358 | 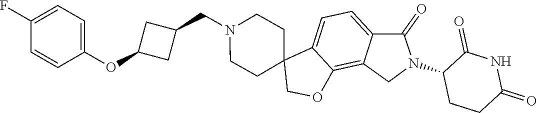 [SMILES: O=C1CC[C@H](N2Cc3c(ccc4c3OCC43CCN(C[C@H]4C[C@@H](Oc5ccc(F)cc5)C4)CC3)C2=O)C(=O)N1] | (S)-3-(1′-(((1s,3R)-3-(4- fluorophenoxy)cyclobutyl)methyl)-6- oxo-6,8-dihydro- 2H,7H-spiro[furo[2,3-e]isoindole-3,4′- piperidin]-7-yl)piperidine-2,6-dione |
| A359 | 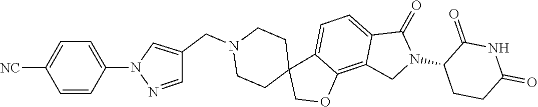 [SMILES: N#Cc1ccc(-n2cc(CN3CCC4(CC3)COc3c4ccc4c3CN([C@H]3CCC(=O)NC3=O)C4=O)cn2)cc1] | (S)-4-(4-((7-(2,6-dioxopiperidin-3-yl)- 6-oxo-7,8-dihydro-2H,6H- spiro[furo[2,3-e]isoindole-3,4′- piperidin]-1′-yl)methyl)-1H-pyrazol-1- yl)benzonitrile |
| A360 | 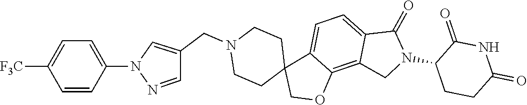 [SMILES: O=C1CC[C@H](N2Cc3c(ccc4c3OCC43CCN(Cc4cnn(-c5ccc(C(F)(F)F)cc5)c4)CC3)C2=O)C(=O)N1] | (S)-3-(6-oxo-1′-((1-(4- (trifluoromethyl)phenyl)-1H-pyrazol- 4-yl)methyl)-6,8-dihydro- 2H,7H-spiro[furo[2,3-e]isoindole-3,4′- piperidin]-7-yl)piperidine-2,6-dione |
| A361 | 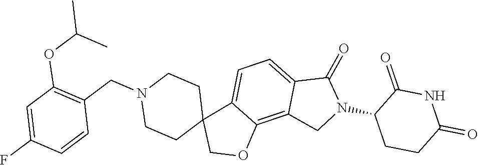 [SMILES: CC(C)Oc1cc(F)ccc1CN1CCC2(CC1)COc1c2ccc2c1CN([C@H]1CCC(=O)NC1=O)C2=O] | (S)-3-(1′-(4-fluoro-2- isopropoxybenzyl)-6-oxo-6,8-dihydro- 2H,7H-spiro[furo[2,3-e]isoindole-3,4′- piperidin]-7-yl)piperidine-2,6-dione |
| A362 | 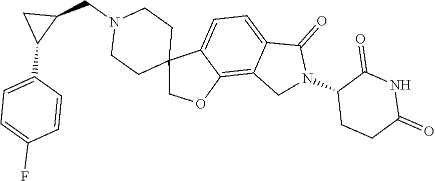 [SMILES: O=C1CC[C@H](N2Cc3c(ccc4c3OCC43CCN(C[C@@H]4C[C@H]4c4ccc(F)cc4)CC3)C2=O)C(=O)N1] | (S)-3-(1′-(((1R,2R)-2-(4- fluorophenyl)cyclopropyl)methyl)-6- oxo-6,8-dihydro- 2H,7H-spiro[furo[2,3-e]isoindole-3,4′- piperidin]-7-yl)piperidine-2,6-dione |

TABLE 2

| No. | Structure | Name |
|---|---|---|
| B1 | 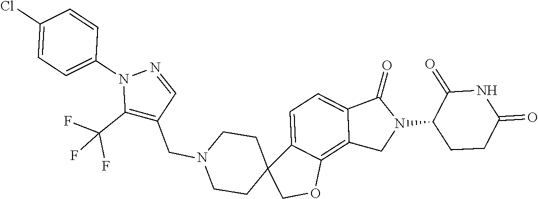 [SMILES: O=C1CC[C@H](N2Cc3c(ccc4c3OCC43CCN(Cc4cnn(-c5ccc(Cl)cc5)c4C(F)(F)F)CC3)C2=O)C(=O)N1] | (S)-3-(1′-((1-(4-chlorophenyl)-5- (trifluoromethyl)-1H-pyrazol-4- yl)methyl)-6-oxo-6,8-dihydro- 2H,7H-spiro[furo[2,3-e]isoindole- 3,4′-piperidin]-7-yl)piperidine-2,6- dione |
| B2 | 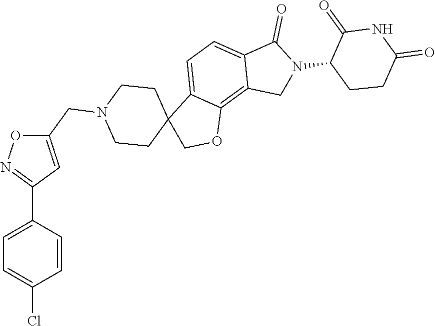 [SMILES: O=C1CC[C@H](N2Cc3c(ccc4c3OCC43CCN(Cc4cc(-c5ccc(Cl)cc5)no4)CC3)C2=O)C(=O)N1] | (S)-3-(1′-((3-(4- chlorophenyl)isoxazol-5- yl)methyl)-6-oxo-6,8-dihydro- 2H,7H-spiro[furo[2,3-e]isoindole- 3,4′-piperidin]-7-yl)piperidine-2,6- dione |
| B3 | 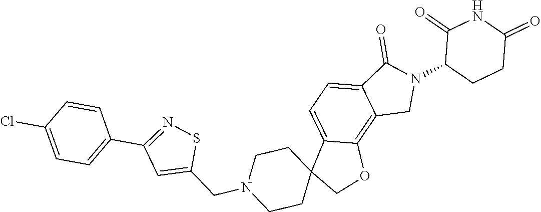 [SMILES: O=C1CC[C@H](N2Cc3c(ccc4c3OCC43CCN(Cc4cc(-c5ccc(Cl)cc5)ns4)CC3)C2=O)C(=O)N1] | (S)-3-(1′-((3-(4- chlorophenyl)isothiazol-5- yl)methyl)-6-oxo-6,8-dihydro- 2H,7H-spiro[furo[2,3-e]isoindole- 3,4′-piperidin]-7-yl)piperidine-2,6- dione |
| B4 | 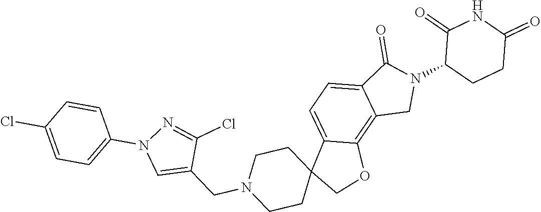 [SMILES: O=C1CC[C@H](N2Cc3c(ccc4c3OCC43CCN(Cc4cn(-c5ccc(Cl)cc5)nc4Cl)CC3)C2=O)C(=O)N1] | (S)-3-(1′-((3-chloro-1-(4- chlorophenyl)-1H-pyrazol-4- yl)methyl)-6-oxo-6,8-dihydro- 2H,7H-spiro[furo[2,3-e]isoindole- 3,4′-piperidin]-7-yl)piperidine-2,6- dione |
| B5 | 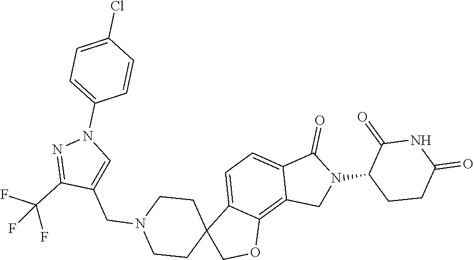 [SMILES: O=C1CC[C@H](N2Cc3c(ccc4c3OCC43CCN(Cc4cn(-c5ccc(Cl)cc5)nc4C(F)(F)F)CC3)C2=O)C(=O)N1] | (S)-3-(1′-((1-(4-chlorophenyl)-3- (trifluoromethyl)-1H-pyrazol-4- yl)methyl)-6-oxo-6,8-dihydro- 2H,7H-spiro[furo[2,3-e]isoindole- 3,4′-piperidin]-7-yl)piperidine-2,6- dione |
| B6 | 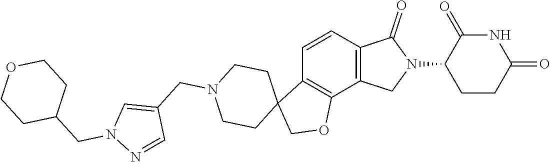 [SMILES: O=C1CC[C@H](N2Cc3c(ccc4c3OCC43CCN(Cc4cnn(CC5CCOCC5)c4)CC3)C2=O)C(=O)N1] | (S)-3-(6-oxo-1′-((1-((tetrahydro- 2H-pyran-4-yl)methyl)-1H- pyrazol-4-yl)methyl)-6,8-dihydro- 2H,7H-spiro[furo[2,3-e]isoindole- 3,4′-piperidin]-7-yl)piperidine-2,6- dione |
| B7 | 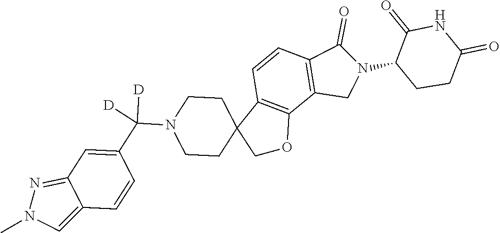 [SMILES: [2H]C([2H])(c1ccc2cn(C)nc2c1)N1CCC2(CC1)COc1c2ccc2c1CN([C@H]1CCC(=O)NC1=O)C2=O] | (S)-3-(1′-((2-methyl-2H-indazol-6- yl)methyl-d2)-6-oxo-6,8-dihydro- 2H,7H-spiro[furo[2,3-e]isoindole- 3,4′-piperidin]-7-yl)piperidine-2,6- dione |
| B8 | 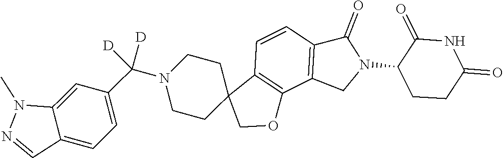 [SMILES: [2H]C([2H])(c1ccc2cnn(C)c2c1)N1CCC2(CC1)COc1c2ccc2c1CN([C@H]1CCC(=O)NC1=O)C2=O] | (S)-3-(1′-((1-methyl-1H-indazol-6- yl)methyl-d2)-6-oxo-6,8-dihydro- 2H,7H-spiro[furo[2,3-e]isoindole- 3,4′-piperidin]-7-yl)piperidine-2,6- dione |
| B9 | 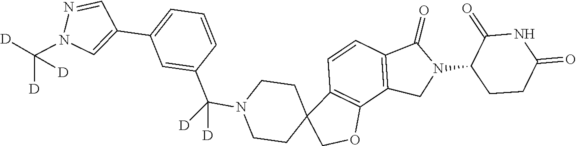 [SMILES: [2H]C([2H])(c1cccc(-c2cnn(C([2H])([2H])[2H])c2)c1)N1CCC2(CC1)COc1c2ccc2c1CN([C@H]1CCC(=O)NC1=O)C2=O] | (S)-3-(1′-((3-(1-(methyl-d3)-1H- pyrazol-4-yl)phenyl)methyl-d2)-6- oxo-6,8-dihydro-2H,7H- spiro[furo[2,3-e]isoindole-3,4′- piperidin]-7-yl)piperidine-2,6- dione |
| B10 | 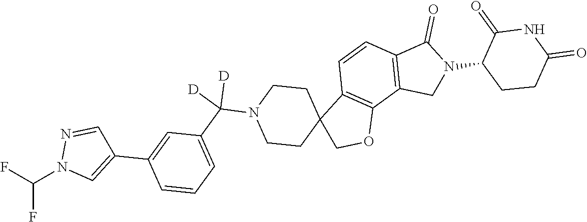 [SMILES: [2H]C([2H])(c1cccc(-c2cnn(C(F)F)c2)c1)N1CCC2(CC1)COc1c2ccc2c1CN([C@H]1CCC(=O)NC1=O)C2=O] | (S)-3-(1′-((3-(1-(difluoromethyl)- 1H-pyrazol-4-yl)phenyl)methyl- d2)-6-oxo-6,8-dihydro-2H,7H- spiro[furo[2,3-e]isoindole-3,4′- piperidin]-7-yl)piperidine-2,6- dione |
| B11 | 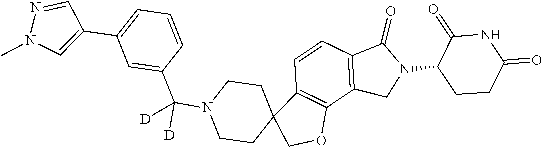 [SMILES: [2H]C([2H])(c1cccc(-c2cnn(C)c2)c1)N1CCC2(CC1)COc1c2ccc2c1CN([C@H]1CCC(=O)NC1=O)C2=O] | (S)-3-(1′-((3-(1-methyl-1H- pyrazol-4-yl)phenyl)methyl-d2)-6- oxo-6,8-dihydro-2H,7H- spiro[furo[2,3-e]isoindole-3,4′- piperidin]-7-yl)piperidine-2,6- dione |
| B12 | 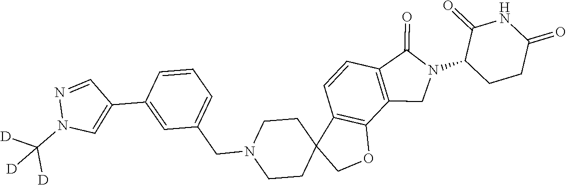 [SMILES: [2H]C([2H])([2H])n1cc(-c2cccc(CN3CCC4(CC3)COc3c4ccc4c3CN([C@H]3CCC(=O)NC3=O)C4=O)c2)cn1] | (S)-3-(1′-(3-(1-(methyl-d3)-1H- pyrazol-4-yl)benzyl)-6-oxo-6,8- dihydro-2H,7H-spiro[furo[2,3- e]isoindole-3,4′-piperidin]-7- yl)piperidine-2,6-dione |
| B13 | 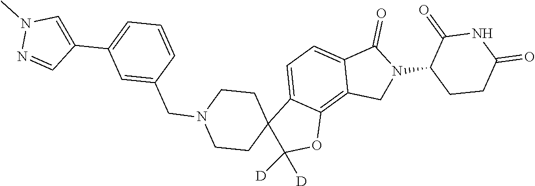 [SMILES: [2H]C1([2H])Oc2c(ccc3c2CN([C@H]2CCC(=O)NC2=O)C3=O)C12CCN(Cc1cccc(-c3cnn(C)c3)c1)CC2] | (S)-3-(1′-(3-(1-methyl-1H-pyrazol- 4-yl)benzyl)-6-oxo-6,8-dihydro- 2H,7H-spiro[furo[2,3-e]isoindole- 3,4′-piperidin]-7-yl-2,2- d2)piperidine-2,6-dione |
| B14 | 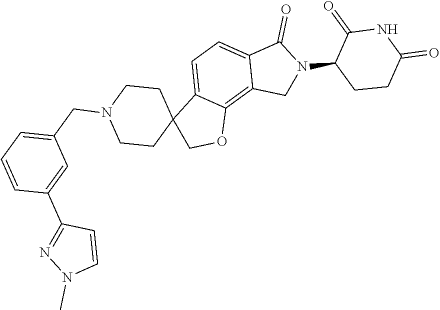 [SMILES: Cn1ccc(-c2cccc(CN3CCC4(CC3)COc3c4ccc4c3CN([C@@H]3CCC(=O)NC3=O)C4=O)c2)n1] | (R)-3-(1′-(3-(1-methyl-1H-pyrazol- 4-yl)benzyl)-6-oxo-6,8-dihydro- 2H,7H-spiro[furo[2,3-e]isoindole- 3,4′-piperidin]-7-yl)piperidine-2,6- dione |
| B15 | 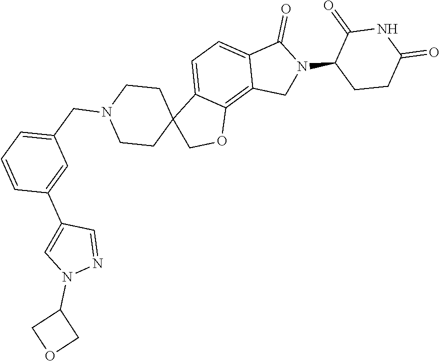 [SMILES: O=C1CC[C@@H](N2Cc3c(ccc4c3OCC43CCN(Cc4cccc(-c5cnn(C6COC6)c5)c4)CC3)C2=O)C(=O)N1] | (R)-3-(1′-(3-(1-(oxetan-3-yl)-1H- pyrazol-4-yl)benzyl)-6-oxo-6,8- dihydro-2H,7H-spiro[furo[2,3- e]isoindole-3,4′-piperidin]-7- yl)piperidine-2,6-dione |
| B16 | 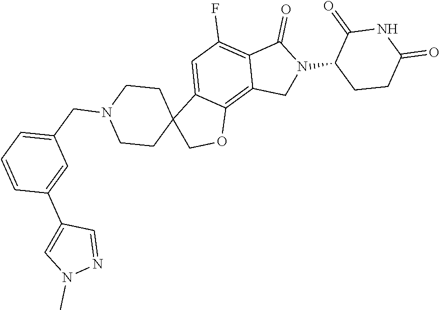 [SMILES: Cn1cc(-c2cccc(CN3CCC4(CC3)COc3c4cc(F)c4c3CN([C@H]3CCC(=O)NC3=O)C4=O)c2)cn1] | (S)-3-(5-fluoro-1′-(3-(1-methyl- 1H-pyrazol-4-yl)benzyl)-6-oxo- 6,8-dihydro-2H,7H-spiro[furo[2,3- e]isoindole-3,4′-piperidin]-7- yl)piperidine-2,6-dione |
| B17 | 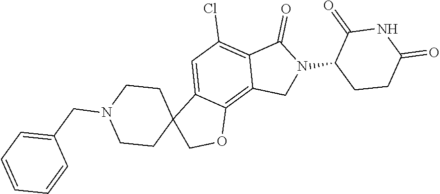 [SMILES: O=C1CC[C@H](N2Cc3c4c(cc(Cl)c3C2=O)C2(CCN(Cc3ccccc3)CC2)CO4)C(=O)N1] | (S)-3-(1′-benzyl-5-chloro-6-oxo- 6,8-dihydro-2H,7H-spiro[furo[2,3- e]isoindole-3,4′-piperidin]-7- yl)piperidine-2,6-dione |
| B18 | 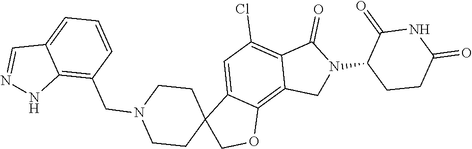 [SMILES: O=C1CC[C@H](N2Cc3c4c(cc(Cl)c3C2=O)C2(CCN(Cc3cccc5cn[nH]c35)CC2)CO4)C(=O)N1] | (S)-3-(1′-((1H-indazol-7- yl)methyl)-5-chloro-6-oxo-6,8- dihydro-2H,7H-spiro[furo[2,3- e]isoindole-3,4′-piperidin]-7- yl)piperidine-2,6-dione |
| B19 | 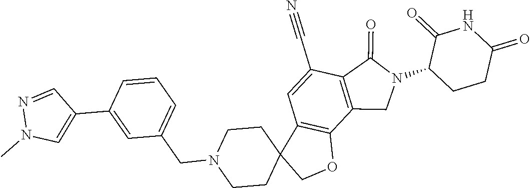 [SMILES: Cn1cc(-c2cccc(CN3CCC4(CC3)COc3c4cc(C#N)c4c3CN([C@H]3CCC(=O)NC3=O)C4=O)c2)cn1] | (S)-7-(2,6-dioxopiperidin-3-yl)-1′- (3-(1-methyl-1H-pyrazol-4- yl)benzyl)-6-oxo-7,8-dihydro- 2H,6H-spiro[furo[2,3-e]isoindole- 3,4′-piperidine]-5-carbonitrile |
| B20 | 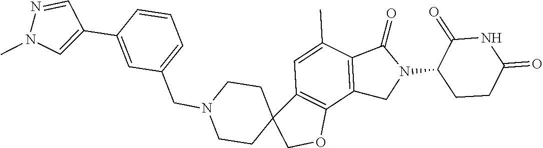 [SMILES: Cc1cc2c(c3c1C(=O)N([C@H]1CCC(=O)NC1=O)C3)OCC21CCN(Cc2cccc(-c3cnn(C)c3)c2)CC1] | (S)-3-(5-methyl-1′-(3-(1-methyl- 1H-pyrazol-4-yl)benzyl)-6-oxo- 6,8-dihydro-2H,7H-spiro[furo[2,3- e]isoindole-3,4′-piperidin]-7- yl)piperidine-2,6-dione |
| B21 | 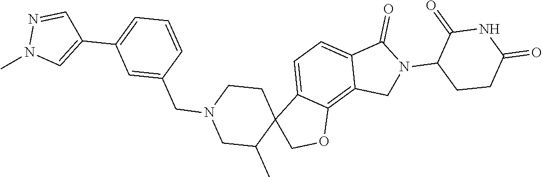 [SMILES: CC1CN(Cc2cccc(-c3cnn(C)c3)c2)CCC12COc1c2ccc2c1CN(C1CCC(=O)NC1=O)C2=O] | 3-(3′-methyl-1′-(3-(1-methyl-1H- pyrazol-4-yl)benzyl)-6-oxo-6,8- dihydro-2H,7H-spiro[furo[2,3- e]isoindole-3,4′-piperidin]-7- yl)piperidine-2,6-dione |
| B22 | 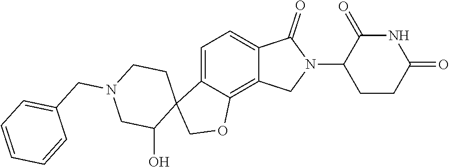 [SMILES: O=C1CCC(N2Cc3c(ccc4c3OCC43CCN(Cc4ccccc4)CC3O)C2=O)C(=O)N1] | 3-(1′-benzyl-3′-hydroxy-6-oxo-6,8- dihydro-2H,7H-spiro[furo[2,3- e]isoindole-3,4′-piperidin]-7- yl)piperidine-2,6-dione |
| B23 | 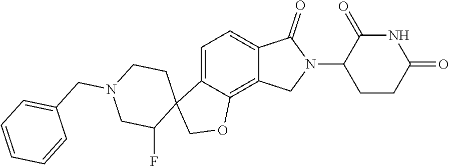 [SMILES: O=C1CCC(N2Cc3c(ccc4c3OCC43CCN(Cc4ccccc4)CC3F)C2=O)C(=O)N1] | 3-(1′-benzyl-3′-fluoro-6-oxo-6,8- dihydro-2H,7H-spiro[furo[2,3- e]isoindole-3,4′-piperidin]-7- yl)piperidine-2,6-dione |
| B24 | 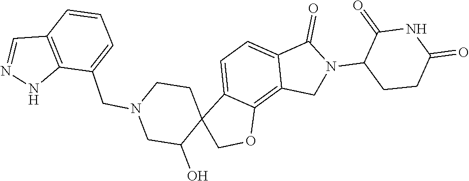 [SMILES: O=C1CCC(N2Cc3c(ccc4c3OCC43CCN(Cc4cccc5cn[nH]c45)CC3O)C2=O)C(=O)N1] | 3-(1′-((1H-indazol-7-yl)methyl)-3′- hydroxy-6-oxo-6,8-dihydro- 2H,7H-spiro[furo[2,3-e]isoindole- 3,4′-piperidin]-7-yl)piperidine-2,6- dione |
| B25 | 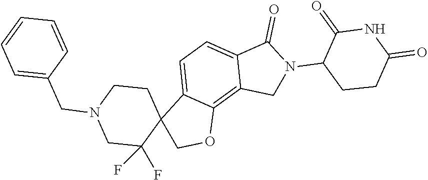 [SMILES: O=C1CCC(N2Cc3c(ccc4c3OCC43CCN(Cc4ccccc4)CC3(F)F)C2=O)C(=O)N1] | 3-(1′-benzyl-3′,3′-difluoro-6-oxo- 6,8-dihydro-2H,7H-spiro[furo[2,3- e]isoindole-3,4′-piperidin]-7- yl)piperidine-2,6-dione |
| B26 | 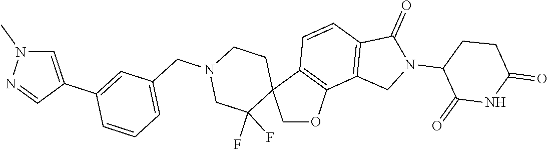 [SMILES: Cn1cc(-c2cccc(CN3CCC4(COc5c4ccc4c5CN(C5CCC(=O)NC5=O)C4=O)C(F)(F)C3)c2)cn1] | 3-(3′,3′-difluoro-1′-(3-(1-methyl- 1H-pyrazol-4-yl)benzyl)-6-oxo- 6,8-dihydro-2H,7H-spiro[furo[2,3- e]isoindole-3,4′-piperidin]-7- yl)piperidine-2,6-dione |
| B27 | 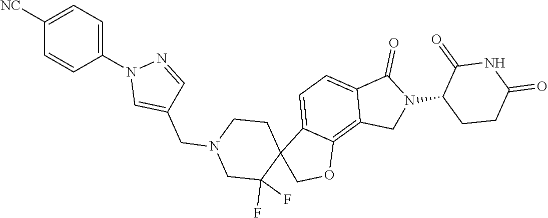 [SMILES: N#Cc1ccc(-n2cc(CN3CCC4(COc5c4ccc4c5CN([C@H]5CCC(=O)NC5=O)C4=O)C(F)(F)C3)cn2)cc1] | 4-(4-((7-((S)-2,6-dioxopiperidin-3- yl)-3′,3′-difluoro-6-oxo-7,8- dihydro-2H,6H-spiro[furo[2,3- e]isoindole-3,4′-piperidin]-1′- yl)methyl)-1H-pyrazol-1- yl)benzonitrile |
| B28 | 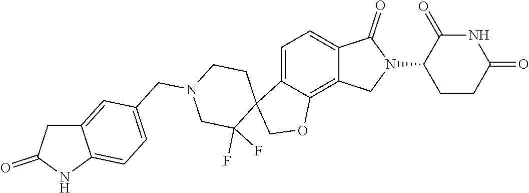 [SMILES: O=C1CC[C@H](N2Cc3c(ccc4c3OCC43CCN(Cc4ccc5c(c4)CC(=O)N5)CC3(F)F)C2=O)C(=O)N1] | (3S)-3-(3′,3′-difluoro-6-oxo-1′-((2- oxoindolin-5-yl)methyl)-6,8- dihydro-2H,7H-spiro[furo[2,3- e]isoindole-3,4′-piperidin]-7- yl)piperidine-2,6-dione |
| B29 | 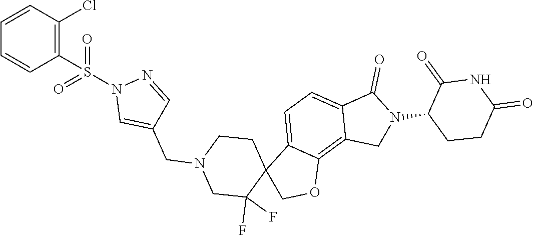 [SMILES: O=C1CC[C@H](N2Cc3c(ccc4c3OCC43CCN(Cc4cnn(S(=O)(=O)c5ccccc5Cl)c4)CC3(F)F)C2=O)C(=O)N1] | (3S)-3-(1′-((1-((2- chlorophenyl)sulfonyl)-1H- pyrazol-4-yl)methyl)-3′,3′-difluoro- 6-oxo-6,8-dihydro-2H,7H- spiro[furo[2,3-e]isoindole-3,4′- piperidin]-7-yl)piperidine-2,6- dione |
| B30 | 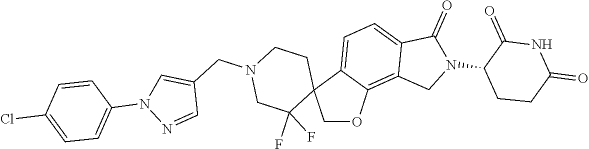 [SMILES: O=C1CC[C@H](N2Cc3c(ccc4c3OCC43CCN(Cc4cnn(-c5ccc(Cl)cc5)c4)CC3(F)F)C2=O)C(=O)N1] | (3S)-3-(1′-((1-(4-chlorophenyl)- 1H-pyrazol-4-yl)methyl)-3′,3′- difluoro-6-oxo-6,8-dihydro-2H,7H- spiro[furo[2,3-e]isoindole-3,4′- piperidin]-7-yl)piperidine-2,6- dione |
| B31 | 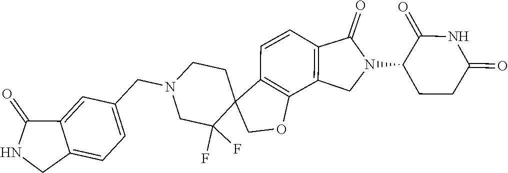 [SMILES: O=C1CC[C@H](N2Cc3c(ccc4c3OCC43CCN(Cc4ccc5c(c4)C(=O)NC5)CC3(F)F)C2=O)C(=O)N1] | (3S)-3-(3′,3′-difluoro-6-oxo-1′-((3- oxoisoindolin-5-yl)methyl)-6,8- dihydro-2H,7H-spiro[furo[2,3- e]isoindole-3,4′-piperidin]-7- yl)piperidine-2,6-dione |
| B32 | 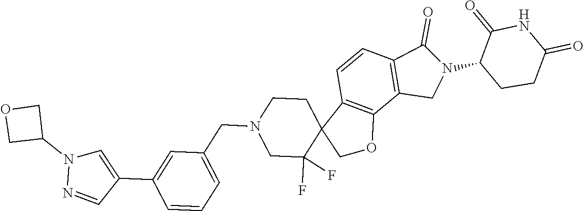 [SMILES: O=C1CC[C@H](N2Cc3c(ccc4c3OCC43CCN(Cc4cccc(-c5cnn(C6COC6)c5)c4)CC3(F)F)C2=O)C(=O)N1] | (3S)-3-(3′,3′-difluoro-1′-(3-(1- (oxetan-3-yl)-1H-pyrazol-4- yl)benzyl)-6-oxo-6,8-dihydro- 2H,7H-spiro[furo[2,3-e]isoindole- 3,4′-piperidin]-7-yl)piperidine-2,6- dione |
| B33 | 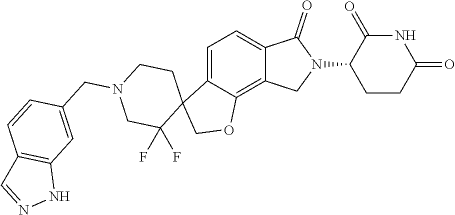 [SMILES: O=C1CC[C@H](N2Cc3c(ccc4c3OCC43CCN(Cc4ccc5cn[nH]c5c4)CC3(F)F)C2=O)C(=O)N1] | (3S)-3-(1′-((1H-indazol-6- yl)methyl)-3′,3′-difluoro-6-oxo- 6,8-dihydro-2H,7H-spiro[furo[2,3- e]isoindole-3,4′-piperidin]-7- yl)piperidine-2,6-dione |
| B34 | 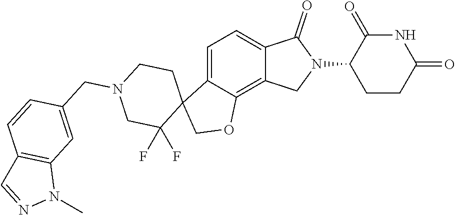 [SMILES: Cn1ncc2ccc(CN3CCC4(COc5c4ccc4c5CN([C@H]5CCC(=O)NC5=O)C4=O)C(F)(F)C3)cc21] | (3S)-3-(3′,3′-difluoro-1′-((1- methyl-1H-indazol-6-yl)methyl)-6- oxo-6,8-dihydro-2H,7H- spiro[furo[2,3-e]isoindole-3,4′- piperidin]-7-yl)piperidine-2,6- dione |
| B35 | 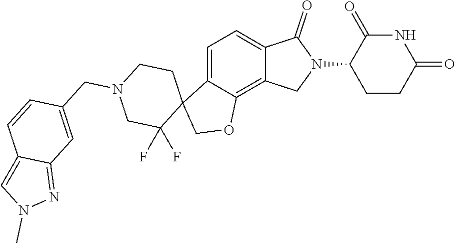 [SMILES: Cn1cc2ccc(CN3CCC4(COc5c4ccc4c5CN([C@H]5CCC(=O)NC5=O)C4=O)C(F)(F)C3)cc2n1] | (3S)-3-(3′,3′-difluoro-1′-((2- methyl-2H-indazol-6-yl)methyl)-6- oxo-6,8-dihydro-2H,7H- spiro[furo[2,3-e]isoindole-3,4′- piperidin]-7-yl)piperidine-2,6- dione |
| B36 | 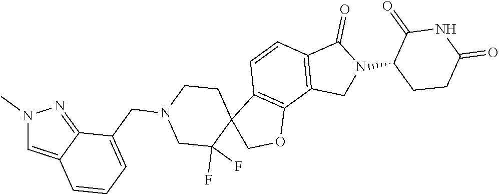 [SMILES: Cn1cc2cccc(CN3CCC4(COc5c4ccc4c5CN([C@H]5CCC(=O)NC5=O)C4=O)C(F)(F)C3)c2n1] | (3S)-3-(3′,3′-difluoro-1′-((2- methyl-2H-indazol-7-yl)methyl)-6- oxo-6,8-dihydro-2H,7H- spiro[furo[2,3-e]isoindole-3,4′- piperidin]-7-yl)piperidine-2,6- dione |
| B37 | 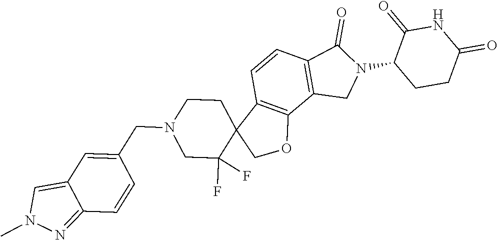 [SMILES: Cn1cc2cc(CN3CCC4(COc5c4ccc4c5CN([C@H]5CCC(=O)NC5=O)C4=O)C(F)(F)C3)ccc2n1] | (3S)-3-(3′,3′-difluoro-1′-((2- methyl-2H-indazol-5-yl)methyl)-6- oxo-6,8-dihydro-2H,7H- spiro[furo[2,3-e]isoindole-3,4′- piperidin]-7-yl)piperidine-2,6- dione |
| B38 | 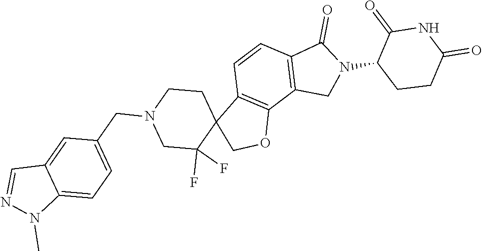 [SMILES: Cn1ncc2cc(CN3CCC4(COc5c4ccc4c5CN([C@H]5CCC(=O)NC5=O)C4=O)C(F)(F)C3)ccc21] | (3S)-3-(3′,3′-difluoro-1′-((1- methyl-1H-indazol-5-yl)methyl)-6- oxo-6,8-dihydro-2H,7H- spiro[furo[2,3-e]isoindole-3,4′- piperidin]-7-yl)piperidine-2,6- dione |
| B39 | 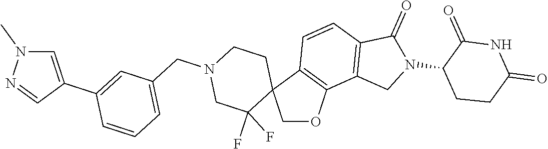 [SMILES: Cn1cc(-c2cccc(CN3CCC4(COc5c4ccc4c5CN([C@H]5CCC(=O)NC5=O)C4=O)C(F)(F)C3)c2)cn1] | (3S)-3-(3′,3′-difluoro-1′-(3-(1- methyl-1H-pyrazol-4-yl)benzyl)-6- oxo-6,8-dihydro-2H,7H- spiro[furo[2,3-e]isoindole-3,4′- piperidin]-7-yl)piperidine-2,6- dione |
| B40 | 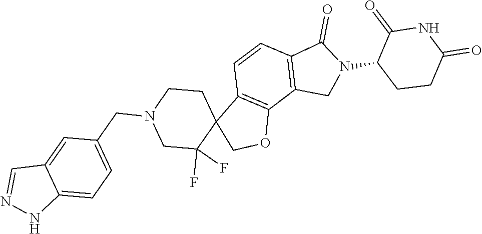 [SMILES: O=C1CC[C@H](N2Cc3c(ccc4c3OCC43CCN(Cc4ccc5[nH]ncc5c4)CC3(F)F)C2=O)C(=O)N1] | (3S)-3-(1′-((1H-indazol-5- yl)methyl)-3′,3′-difluoro-6-oxo- 6,8-dihydro-2H,7H-spiro[furo[2,3- e]isoindole-3,4′-piperidin]-7- yl)piperidine-2,6-dione |
| B41 | 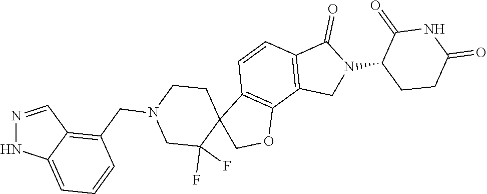 [SMILES: O=C1CC[C@H](N2Cc3c(ccc4c3OCC43CCN(Cc4cccc5[nH]ncc45)CC3(F)F)C2=O)C(=O)N1] | (3S)-3-(1′-((1H-indazol-4- yl)methyl)-3′,3′-difluoro-6-oxo- 6,8-dihydro-2H,7H-spiro[furo[2,3- e]isoindole-3,4′-piperidin]-7- yl)piperidine-2,6-dione |
| B42 | 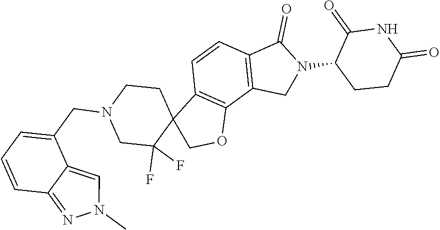 [SMILES: Cn1cc2c(CN3CCC4(COc5c4ccc4c5CN([C@H]5CCC(=O)NC5=O)C4=O)C(F)(F)C3)cccc2n1] | (3S)-3-(3′,3′-difluoro-1′-((2- methyl-2H-indazol-4-yl)methyl)-6- oxo-6,8-dihydro-2H,7H- spiro[furo[2,3-e]isoindole-3,4′- piperidin]-7-yl)piperidine-2,6- dione |
| B43 | 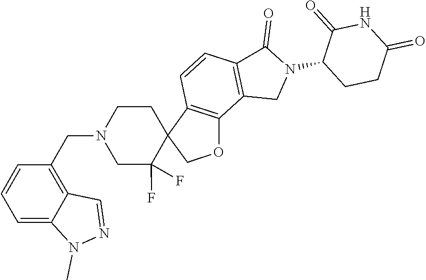 [SMILES: Cn1ncc2c(CN3CCC4(COc5c4ccc4c5CN([C@H]5CCC(=O)NC5=O)C4=O)C(F)(F)C3)cccc21] | (3S)-3-(3′,3′-difluoro-1′-((1- methyl-1H-indazol-4-yl)methyl)-6- oxo-6,8-dihydro-2H,7H- spiro[furo[2,3-e]isoindole-3,4′- piperidin]-7-yl)piperidine-2,6- dione |
| B44 | 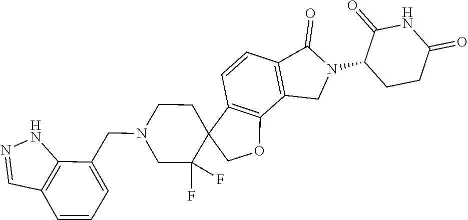 [SMILES: O=C1CC[C@H](N2Cc3c(ccc4c3OCC43CCN(Cc4cccc5cn[nH]c45)CC3(F)F)C2=O)C(=O)N1] | (3S)-3-(1′-((1H-indazol-7- yl)methyl)-3′,3′-difluoro-6-oxo- 6,8-dihydro-2H,7H-spiro[furo[2,3- e]isoindole-3,4′-piperidin]-7- yl)piperidine-2,6-dione |
| B45 | 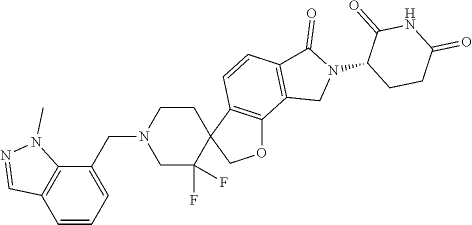 [SMILES: Cn1ncc2cccc(CN3CCC4(COc5c4ccc4c5CN([C@H]5CCC(=O)NC5=O)C4=O)C(F)(F)C3)c21] | (3S)-3-(3′,3′-difluoro-1′-((1- methyl-1H-indazol-7-yl)methyl)-6- oxo-6,8-dihydro-2H,7H- spiro[furo[2,3-e]isoindole-3,4′- piperidin]-7-yl)piperidine-2,6- dione |
| B46 | 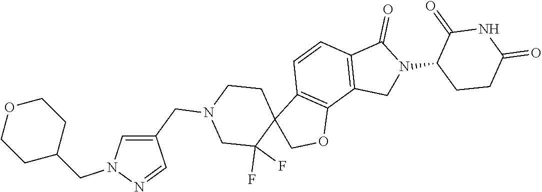 [SMILES: O=C1CC[C@H](N2Cc3c(ccc4c3OCC43CCN(Cc4cnn(CC5CCOCC5)c4)CC3(F)F)C2=O)C(=O)N1] | (3S)-3-(3′,3′-difluoro-6-oxo-1′-((1- ((tetrahydro-2H-pyran-4- yl)methyl)-1H-pyrazol-4- yl)methyl)-6,8-dihydro-2H,7H- spiro[furo[2,3-e]isoindole-3,4′- piperidin]-7-yl)piperidine-2,6- dione |
| B47 | 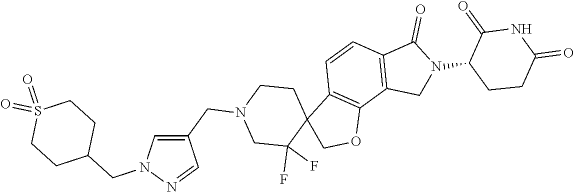 [SMILES: O=C1CC[C@H](N2Cc3c(ccc4c3OCC43CCN(Cc4cnn(CC5CCS(=O)(=O)CC5)c4)CC3(F)F)C2=O)C(=O)N1] | (3S)-3-(1′-((1-((1,1- dioxidotetrahydro-2H-thiopyran-4- yl)methyl)-1H-pyrazol-4- yl)methyl)-3′,3′-difluoro-6-oxo- 6,8-dihydro-2H,7H-spiro[furo[2,3- e]isoindole-3,4′-piperidin]-7- yl)piperidine-2,6-dione |
| B48 | 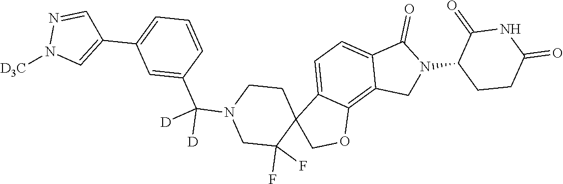 [SMILES: [2H]C([2H])(c1cccc(-c2cnn(C([2H])([2H])[2H])c2)c1)N1CCC2(COc3c2ccc2c3CN([C@H]3CCC(=O)NC3=O)C2=O)C(F)(F)C1] | (3S)-3-(3′,3′-difluoro-1′-((3-(1- (methyl-d3)-1H-pyrazol-4- yl)phenyl)methyl-d2)-6-oxo-6,8- dihydro-2H,7H-spiro[furo[2,3- e]isoindole-3,4′-piperidin]-7- yl)piperidine-2,6-dione |
| B49 | 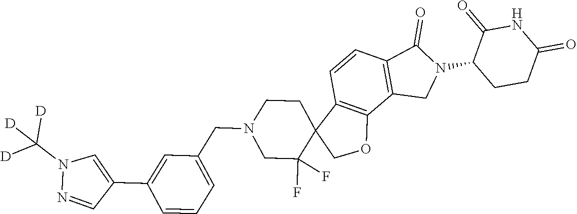 [SMILES: [2H]C([2H])([2H])n1cc(-c2cccc(CN3CCC4(COc5c4ccc4c5CN([C@H]5CCC(=O)NC5=O)C4=O)C(F)(F)C3)c2)cn1] | (3S)-3-(3′,3′-difluoro-1′-(3-(1- (methyl-d3)-1H-pyrazol-4- yl)benzyl)-6-oxo-6,8-dihydro- 2H,7H-spiro[furo[2,3-e]isoindole- 3,4′-piperidin]-7-yl)piperidine-2,6- dione |
| B50 | 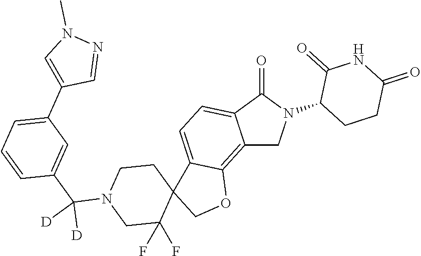 [SMILES: [2H]C([2H])(c1cccc(-c2cnn(C)c2)c1)N1CCC2(COc3c2ccc2c3CN([C@H]3CCC(=O)NC3=O)C2=O)C(F)(F)C1] | (3S)-3-(3′,3′-difluoro-1′-((3-(1- methyl-1H-pyrazol-4- yl)phenyl)methyl-d2)-6-oxo-6,8- dihydro-2H,7H-spiro[furo[2,3- e]isoindole-3,4′-piperidin]-7- yl)piperidine-2,6-dione |
| B51 | 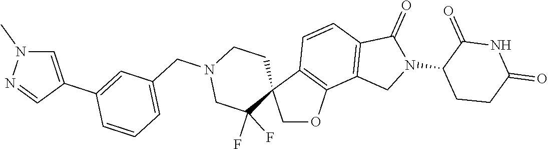 [SMILES: Cn1cc(-c2cccc(CN3CC[C@]4(COc5c4ccc4c5CN([C@H]5CCC(=O)NC5=O)C4=O)C(F)(F)C3)c2)cn1] | (S)-3-((S)-3′,3′-difluoro-1′-(3-(1- methyl-1H-pyrazol-4-yl)benzyl)-6- oxo-6,8-dihydro-2H,7H- spiro[furo[2,3-e]isoindole-3,4′- piperidin]-7-yl)piperidine-2,6- dione |
| B52 | 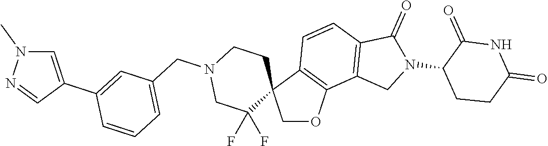 [SMILES: Cn1cc(-c2cccc(CN3CC[C@@]4(COc5c4ccc4c5CN([C@H]5CCC(=O)NC5=O)C4=O)C(F)(F)C3)c2)cn1] | (S)-3-((R)-3′,3′-difluoro-1′-(3-(1- methyl-1H-pyrazol-4-yl)benzyl)-6- oxo-6,8-dihydro-2H,7H- spiro[furo[2,3-e]isoindole-3,4′- piperidin]-7-yl)piperidine-2,6- dione |
| B53 | 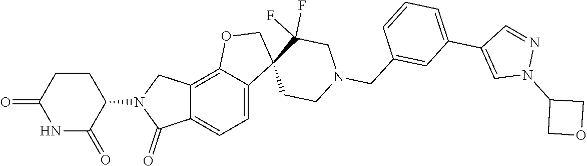 [SMILES: O=C1CC[C@H](N2Cc3c(ccc4c3OC[C@]43CCN(Cc4cccc(-c5cnn(C6COC6)c5)c4)CC3(F)F)C2=O)C(=O)N1] | (S)-3-((S)-3′,3′-difluoro-1′-(3-(1- (oxetan-3-yl)-1H-pyrazol-4- yl)benzyl)-6-oxo-6,8-dihydro- 2H,7H-spiro[furo[2,3-e]isoindole- 3,4′-piperidin]-7-yl)piperidine-2,6- dione |
| B54 | 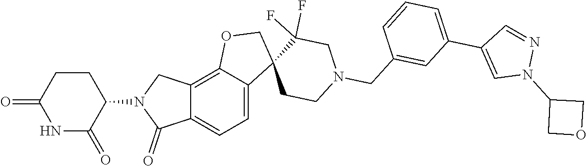 [SMILES: O=C1CC[C@H](N2Cc3c(ccc4c3OC[C@@]43CCN(Cc4cccc(-c5cnn(C6COC6)c5)c4)CC3(F)F)C2=O)C(=O)N1] | (S)-3-((R)-3′,3′-difluoro-1′-(3-(1- (oxetan-3-y1)-1H-pyrazol-4- yl)benzyl)-6-oxo-6,8-dihydro- 2H,7H-spiro[furo[2,3-e]isoindole- 3,4′-piperidin]-7-yl)piperidine-2,6- dione |
| B55 | 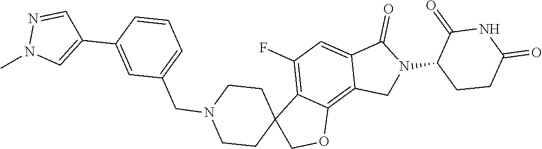 [SMILES: Cn1cc(-c2cccc(CN3CCC4(CC3)COc3c5c(cc(F)c34)C(=O)N([C@H]3CCC(=O)NC3=O)C5)c2)cn1] | (S)-3-(4-fluoro-1′-(3-(1-methyl- 1H-pyrazol-4-yl)benzyl)-6-oxo- 6,8-dihydro-2H,7H-spiro[furo[2,3- e]isoindole-3,4′-piperidin]-7- yl)piperidine-2,6-dione |
| B56 | 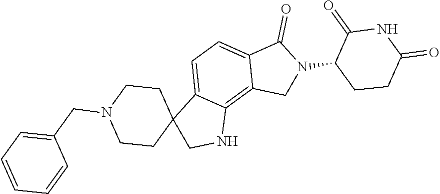 [SMILES: O=C1CC[C@H](N2Cc3c(ccc4c3NCC43CCN(Cc4ccccc4)CC3)C2=O)C(=O)N1] | (S)-3-(1-benzyl-6′-oxo-1′,2′,6′,8′- tetrahydro-7′H-spiro[piperidine- 4,3′-pyrrolo[3,4-g]indol]-7′- yl)piperidine-2,6-dione |
| B57 | 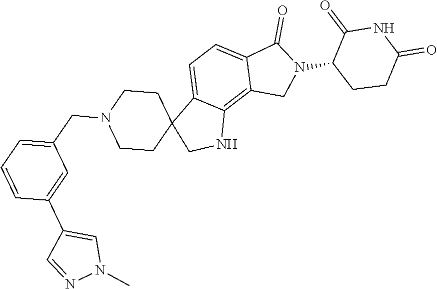 [SMILES: Cn1cc(-c2cccc(CN3CCC4(CC3)CNc3c4ccc4c3CN([C@H]3CCC(=O)NC3=O)C4=O)c2)cn1] | (S)-3-(1-(3-(1-methyl-1H-pyrazol- 4-yl)benzyl)-6′-oxo-1′,2′,6′,8′- tetrahydro-7′H-spiro[piperidine- 4,3′-pyrrolo[3,4-g]indol]-7′- yl)piperidine-2,6-dione |
| B58 | 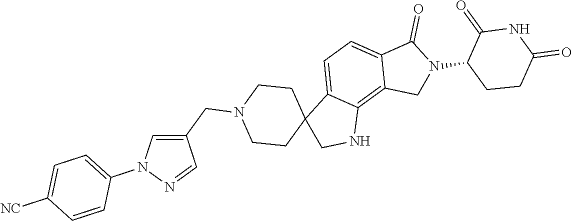 [SMILES: N#Cc1ccc(-n2cc(CN3CCC4(CC3)CNc3c4ccc4c3CN([C@H]3CCC(=O)NC3=O)C4=O)cn2)cc1] | (S)-4-(4-((7′-(2,6-dioxopiperidin-3- yl)-6′-oxo-1′,6′,7′,8′-tetrahydro-2′H- spiro[piperidine-4,3′-pyrrolo[3,4- g]indol]-1-yl)methyl)-1H-pyrazol- 1-yl)benzonitrile |

The compounds of the present disclosure may possess advantageous characteristics, as compared to known compounds, such as known IKZF2 degraders. For example, the compounds of the present disclosure may display more potent estrogen receptor activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure may be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Forms of Compounds Disclosed Herein

### Pharmaceutically Acceptable Salts

In some embodiments, the compounds disclosed herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include alkali or alkaline earth salts, like lithium, sodium, potassium, calcium, magnesium, aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the invention.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present invention.

In some embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers/Stereoisomers

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

In some embodiments, the compounds described herein exist as geometric isomers. In some embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the invention.

In some embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the invention.

In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

In some embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. All tautomeric forms of the compounds disclosed herein are contemplated and are within the scope of the invention. The exact ratio of the tautomers depends on several factors, including temperature, solvent, and pH.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

### Isotopic Species

In some aspects, the present disclosure provides a compound being an isotopic derivative (e.g., isotopically labeled compound) of any one of the compounds disclosed herein.

In some embodiments, the compound is an isotopic derivative of any one of the compounds described in Table 1 or Table 2, or a pharmaceutically acceptable salt thereof.

In some embodiments, the compound is an isotopic derivative of any one of the compounds described in Table 1 or Table 2.

In some embodiments, the compound is an isotopic derivative of any one of the compounds described in Table 1, or a pharmaceutically acceptable salt thereof.

In some embodiments, the compound is an isotopic derivative of any one of the compounds described in Table 1.

In some embodiments, the compound is an isotopic derivative of any one of the compounds described in Table 2, or a pharmaceutically acceptable salt thereof.

In some embodiments, the compound is an isotopic derivative of any one of the compounds described in Table 2.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described in Table 1 or Table 2, or a pharmaceutically acceptable salt thereof.

In some embodiments, the compound is a deuterium labeled compound of any one of the compounds described in Table 1 or Table 2.

In some embodiments, the compound is a deuterium labeled compound of any one of the compounds described in Table 1, or a pharmaceutically acceptable salt thereof.

In some embodiments, the compound is a deuterium labeled compound of any one of the compounds described in Table 1.

In some embodiments, the compound is a deuterium labeled compound of any one of the compounds described in Table 2, or a pharmaceutically acceptable salt thereof.

In some embodiments, the compound is a deuterium labeled compound of any one of the compounds described in Table 2.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the present disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., in the Examples).

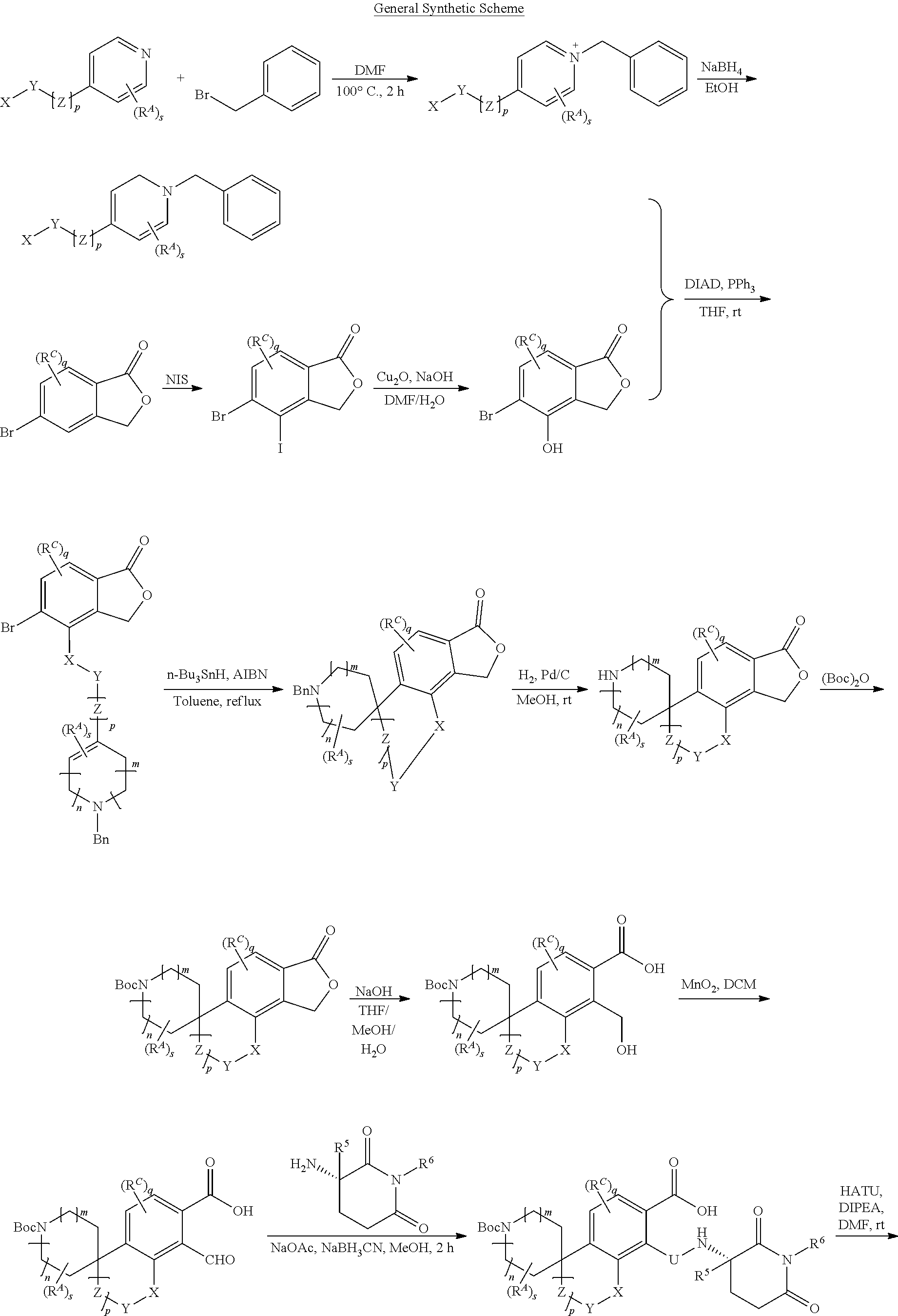

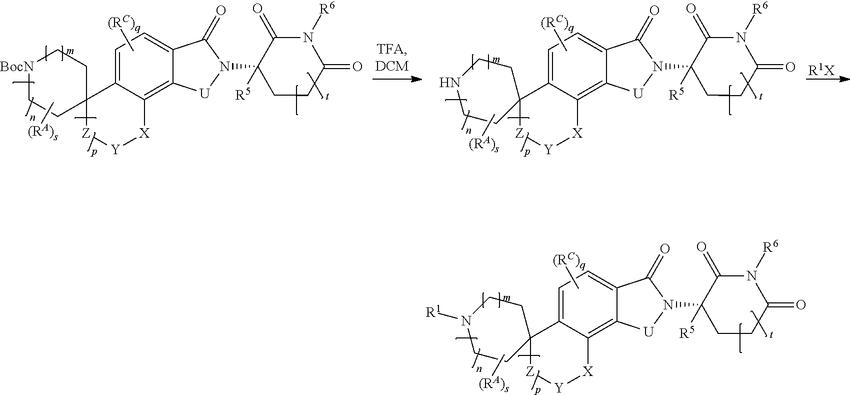

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes. Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Exemplary Biological Assays

The biological activities of the compounds of the present disclosure can be assessed with methods and assays known in the art.

### Exemplary Cereblon Binding Assay

The binding to cereblon (CRBN) is determined using the Cereblon Binding Kit (Cisbio, #64BDCRBNPEG) following the manufacturer's instruction. Briefly, serially diluted compounds are incubated with GST-tagged wild-type human CRBN protein, XL665-labelled Thalidomide and Europium Cryptate labelled GST antibody. Time Resolved Fluorescence Resonance Energy Transfer (TR-FRET) measurements are acquired with, e.g., MARS data analysis software (BMG Labtech. The readings are normalized to the control (0.5%) and the IC₅₀ is calculated by nonlinear regression (four parameters sigmoid fitted with variable slope) analysis using, e.g., the GraphPad Prism 8 software.

### Exemplary Immunoblotting

Cells are maintained in the appropriate culture medium with 10% FBS at 37° C. and an atmosphere of 5% CO₂.

Cells are lysed, resolved by SDS-PAGE, and transferred to a PVDF membrane (Millipore). Membranes are blocked, e.g., using Odyssey TBS Blocker Buffer (LI-COR). Secondary antibodies, e.g., IRDye 680RD and 800CW Dye-labeled are used. The washed membranes are scanned using e.g., an Odyssey CLx imager (LI-COR). The intensity of Western blot signaling is quantitated using the Odyssey software. Primary antibodies used include: Helios (D8W4X) XP® Rabbit mAb (Cell Signaling Technology, #42427) and GAPDH mouse monoclonal antibody (Santa Cruz Biotechnology, sc-47724).

### Exemplary IKZF2 HiBiT Assay

Degradation of IKZF2 protein is determined by IKZF2 HiBiT assay using the Jurkat-IKZF2-HiBiT (Promega) cell line. Briefly, cells are seeded in culture medium. Compounds are serially diluted in culture medium, and certain volume of the diluted compounds is added to the appropriate well of the plate. After the addition of compounds, the cells are incubated. At the end of treatment, Nano-Glo HiBiT Lytic Detection Reagent (Promega) is added to each well, and then the plates are incubated at room temperature for a certain time period. The luminescent signal is measured using a CALRIOstar plate reader (BMG Labtech). The readings are normalized to the DMSO-treated cells and the IC₅₀ is calculated by nonlinear regression (four parameters sigmoid fitted with variable slope, least squares fit, and no constraint) analysis using the GraphPad Prism 8 software.

### Methods of Use

In certain aspects, the present disclosure provides methods of degrading a IKZF2 protein in a subject, comprising administering to the subject a compound disclosed herein.

In certain aspects, the present disclosure provides uses of a compound disclosed herein in the manufacture of a medicament for degrading a IKZF2 protein in a subject.

In certain aspects, the present disclosure provides compounds disclosed herein for use in degrading a IKZF2 protein in a subject.

In certain aspects, the present disclosure provides methods of treating or preventing a disease or disorder in a subject in need thereof, comprising administering to the subject a compound disclosed herein (e.g., in a therapeutically effective amount).

In certain aspects, the present disclosure provides methods of treating a disease or disorder in a subject in need thereof, comprising administering to the subject a compound disclosed herein (e.g., in a therapeutically effective amount).

In certain aspects, the present disclosure provides uses of a compound disclosed herein in the manufacture of a medicament for treating or preventing a disease or disorder in a subject in need thereof.

In certain aspects, the present disclosure provides uses of a compound disclosed herein in the manufacture of a medicament for treating a disease or disorder in a subject in need thereof.

In certain aspects, the present disclosure provides compounds disclosed herein for use in treating or preventing a disease or disorder in a subject in need thereof.

In certain aspects, the present disclosure provides compounds disclosed herein for use in treating a disease or disorder in a subject in need thereof.

In certain embodiments, the disease or disorder is an IKZF2-mediated disease or disorder.

In certain embodiments, the disease or disorder is a cancer.

In certain embodiments, the cancer includes, but is not limited to, one or more of the cancers of Table A.

TABLE A

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial | |
| | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological cancer | hepatoblastoma |
| cancer | | | |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone | mast cell leukemia | mediastinal germ | medullary |
| B-cell lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In certain embodiments, the cancer is a solid tumor. In certain embodiments, the cancer is a hematological cancer. Exemplary hematological cancers include, but are not limited to, the cancers listed in Table B. In certain embodiments, the hematological cancer is acute lymphocytic leukemia, chronic lymphocytic leukemia (including B-cell chronic lymphocytic leukemia), or acute myeloid leukemia.

TABLE B

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In certain embodiments, the disease or disorder is T cell leukemia or T cell lymphoma, Hodgkin's lymphoma or non-Hodgkin's lymphoma, myeloid leukemia, non-small cell lung cancer (NSCLC), melanoma, triple-negative breast cancer (TNBC), nasopharyngeal cancer (NPC), microsatellite stable colorectal cancer (mssCRC), thymoma, carcinoid, or gastrointestinal stromal tumor (GIST).

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In certain embodiments, a heteroalkyl group is a saturated group having 1 to 9 carbon atoms and 1, 2, 3, or 4 heteroatoms (“heteroC₁₋₉ alkyl”). In certain embodiments, a heteroalkyl group is a saturated group having 1 to 8 carbon atoms and 1, 2, 3, or 4 heteroatoms (“heteroC₁₋₈ alkyl”). In certain embodiments, a heteroalkyl group is a saturated group having 1 to 7 carbon atoms and 1, 2, 3, or 4 heteroatoms (“heteroC₁₋₇ alkyl”). In certain embodiments, a heteroalkyl group is a group having 1 to 6 carbon atoms and 1, 2, or 3 heteroatoms (“heteroC₁₋₆ alkyl”). In certain embodiments, a heteroalkyl group is a saturated group having 1 to 5 carbon atoms and 1 or 2 heteroatoms (“heteroC₁₋₅ alkyl”). In certain embodiments, a heteroalkyl group is a saturated group having 1 to 4 carbon atoms and/or 2 heteroatoms (“heteroC₁₋₄ alkyl”). In certain embodiments, a heteroalkyl group is a saturated group having 1 to 3 carbon atoms and 1 heteroatom (“heteroC₁₋₃ alkyl”). In certain embodiments, a heteroalkyl group is a saturated group having 1 to 2 carbon atoms and 1 heteroatom (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In certain embodiments, a heteroalkenyl group has 2 to 9 carbon atoms at least one double bond, and 1, 2, 3, or 4 heteroatoms (“heteroC₂₋₉ alkenyl”). In certain embodiments, a heteroalkenyl group has 2 to 8 carbon atoms, at least one double bond, and 1, 2, 3, or 4 heteroatoms (“heteroC₂₋₈ alkenyl”). In certain embodiments, a heteroalkenyl group has 2 to 7 carbon atoms, at least one double bond, and 1, 2, 3, or 4 heteroatoms (“heteroC₂₋₇ alkenyl”). In certain embodiments, a heteroalkenyl group has 2 to 6 carbon atoms, at least one double bond, and 1, 2, or 3 heteroatoms (“heteroC₂₋₆ alkenyl”). In certain embodiments, a heteroalkenyl group has 2 to 5 carbon atoms, at least one double bond, and 1 or 2 heteroatoms (“heteroC₂₋₅ alkenyl”). In certain embodiments, a heteroalkenyl group has 2 to 4 carbon atoms, at least one double bond, and 1 or 2 heteroatoms (“heteroC₂₋₄ alkenyl”). In certain embodiments, a heteroalkenyl group has 2 to 3 carbon atoms, at least one double bond, and 1 heteroatom (“heteroC₂₋₃ alkenyl”). In certain embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In certain embodiments, a heteroalkynyl group has 2 to 9 carbon atoms, at least one triple bond, and 1, 2, 3, or 4 heteroatoms (“heteroC₂₋₉ alkynyl”). In certain embodiments, a heteroalkynyl group has 2 to 8 carbon atoms, at least one triple bond, and 1, 2, 3, or 4 heteroatoms (“heteroC₂₋₈ alkynyl”). In certain embodiments, a heteroalkynyl group has 2 to 7 carbon atoms, at least one triple bond, and 1, 2, 3, or 4 heteroatoms (“heteroC₂₋₇ alkynyl”). In certain embodiments, a heteroalkynyl group has 2 to 6 carbon atoms, at least one triple bond, and 1, 2, or 3 heteroatoms (“heteroC₂₋₆ alkynyl”). In certain embodiments, a heteroalkynyl group has 2 to 5 carbon atoms, at least one triple bond, and 1 or 2 heteroatoms (“heteroC₂₋₅ alkynyl”). In certain embodiments, a heteroalkynyl group has 2 to 4 carbon atoms, at least one triple bond, and 1 or 2 heteroatoms (“heteroC₂₋₄ alkynyl”). In certain embodiments, a heteroalkynyl group has 2 to 3 carbon atoms, at least one triple bond, and 1 heteroatom (“heteroC₂₋₃ alkynyl”). In certain embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Arylene” as used herein, refers to an aryl group wherein two hydrogens are removed to provide a divalent radical. When a range or number of carbons is provided for a particular “arylene” group, it is understood that the range or number refers to the range or number of carbons in the aryl group. An “arylene” group may be substituted or unsubstituted with one or more substituents as described herein.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroarylene” as used herein, refers to a heteroaryl group wherein two hydrogens are removed to provide a divalent radical. When a range or number of ring members is provided for a particular “heteroarylene” group, it is understood that the range or number refers to the number of ring members in the heteroaryl group. A “heteroarylene” group may be substituted or unsubstituted with one or more substituents as described herein.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Carbocyclylene” as used herein, refers to a carbocyclyl group wherein two hydrogens are removed to provide a divalent radical. The divalent radical may be present on different atoms or the same atom of the carbocyclylene group. When a range or number of carbons is provided for a particular “carbocyclyl” group, it is understood that the range or number refers to the range or number of carbons in the carbocyclyl group. A “carbocyclyl” group may be substituted or unsubstituted with one or more substituents as described herein.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5 membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Heterocyclylene” as used herein, refers to a heterocyclyl group wherein two hydrogens are removed to provide a divalent radical. The divalent radical may be present on different atoms or the same atom of the heterocyclylene group. When a range or number of ring members is provided for a particular “heterocyclylene” group, it is understood that the range or number refers to the number of ring members in the heterocyclylene group. A “heterocyclylene” group may be substituted or unsubstituted with one or more substituents as described herein.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (Cl), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g, t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylactically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

It is also to be understood that compounds that have the same molecular formula but differ in the nature or sequence of bonding of their atoms or the arrangement of their atoms in space are termed “isomers.” Isomers that only differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure (S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### I. Synthetic Routes and Procedures

3-(6-oxo-6,8-dihydro-2H,7H-spiro[furo[2,3-e]isoindole-3,4′-piperidin]-7-yl)piperidine-2,6-dione(C-1)

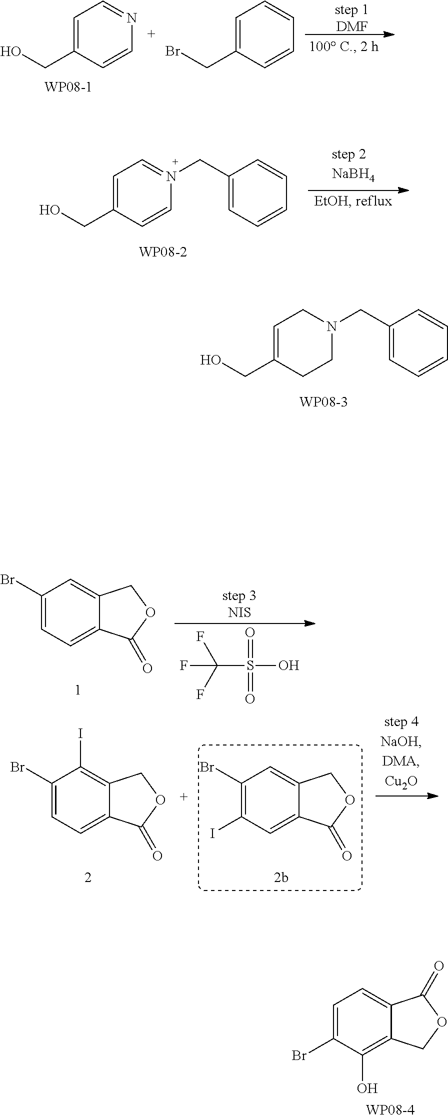

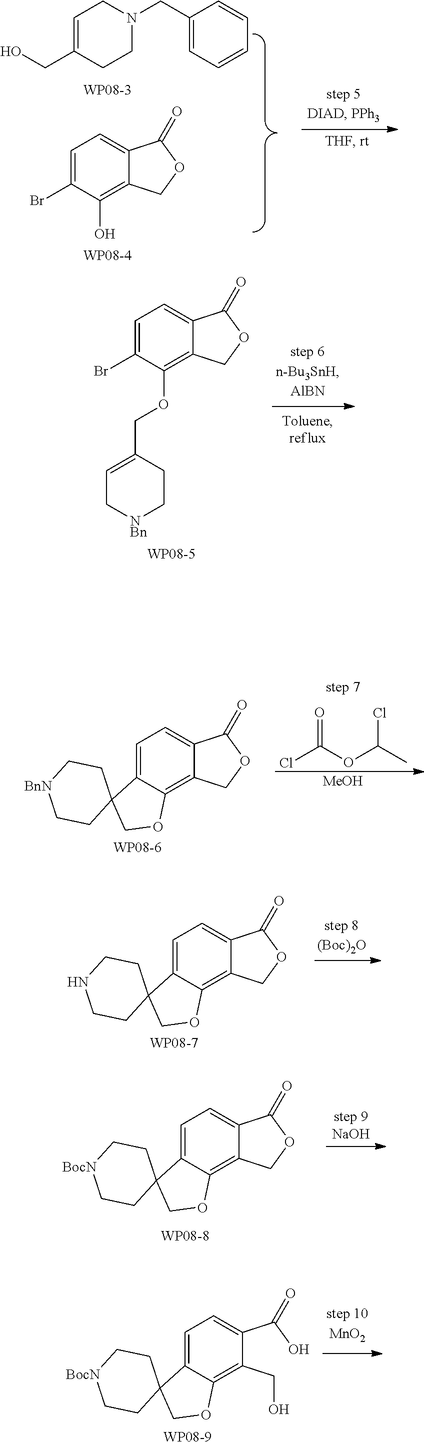

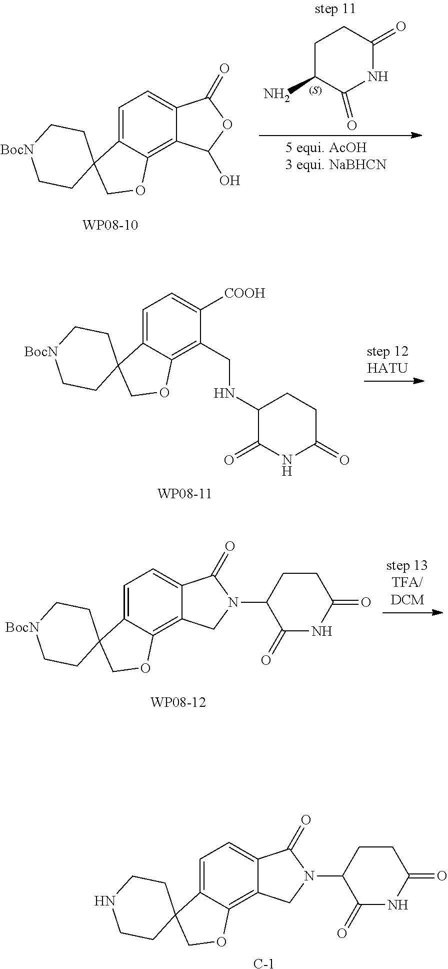

### Step 1-2

To a solution of pyridin-4-ylmethanol (WP08-1, 100 g, 916 mmol, 1.0 eq.) in DMF (400 mL) was added BnBr (172 g, 1.0 mol, 1.1 eq.). The mixture was allowed to heat to 100° C. and stirred 3 h. TLC showed no starting material remained and a new spot formed. The residue was dissolved in EtOH (1500 mL), then 45 g of sodium borohydride (1.19 mol, 1.3 eq.) was added portionwise at 0° C. The mixture was continued to stir at 0° C. for 1 h and then at reflux for 2 h. The solvent was evaporated under reduced pressure, then water was added, and the mixture was extracted with ethyl acetate. The combined organic phases were dried over Na₂SO₄ and evaporated. The residue was purified by flash chromatograph (DCM:MeOH=100:0-30:1) to afford 107 g of product WP08-3 (Viscous oil, 2 steps, yield 80%).

LC/MS (ESI) m/z: 204.14; ¹H NMR (400 MHz, CDCl₃): 7.25-7.41 (m, 5H), 5.59-5.66 (m, 1H), 4.01 (s, 2H), 3.60 (s, 2H), 2.95-3.02 (m, 2H), 2.61 (t, J=5.8 Hz, 2H), 2.36 (s,br, 1H), 2.10-2.21 (m, 2H).

### Step 3

To a solution of 5-Bromo-3H-isobenzofuran-1-one (1) (100 g, 1 eq.) in trifluoromethanesulfonic acid (1000 g, 10 eq) was added NIS (125 g, 1.2 eq.) at 0° C. in portions. The mixture was allowed to warm to room temperature and stirred overnight. TLC showed no starting material remained and two new spots formed. The reaction mixture was poured into ice-water and yellow solid precipitated. The mixture was filtered and the filter cake was washed with ice cold water. The filter cake was dried and obtained as a yellow solid (100 g, yield 62%), to be a mixture of product 2 (top spot on TLC) and product 2b (bottom spot on TLC, which was not further reacted in next step).

LC/MS (ESI) m/z: 337.84; ¹H NMR (400 MHz, CDCl₃): 7.83 (d, J=8.0 Hz, 1H), 7.77 (d, J=8.0 Hz, 1H), 5.10 (s, 2H).

### Step 4

To a mixture of compound 2 (100 g, 1 eq.), sodium hydroxide (57.5 g, 5 eq.) in water (1000 mL, 1.5 M) and N,N-dimethylacetamide (600 mL) was added cuprous oxide (8.5 g, 0.2 eq.). The reaction mixture was heated to 80° C. and stirred for 12 h. TLC showed the compound 2 (top spot on TLC) was completely consumed. The reaction mixture was poured into water (1000 mL) and treated with solid K₂CO₃ until pH 8-9, and extracted with EA. The aqueous layer neutralized using 1 N hydrochloride solution and extracted with ethyl acetate, washed with brine, and then dried over sodium sulfate and evaporated. The crude product was purified by silica gel column chromatography to give compound WP08-4 was obtained as a yellow solid (42 g, 39% yield).

LC/MS (ESI) m/z: 228.94; ¹H NMR (400 MHz, DMSO-d₆) δ 10.90 (s, 1H), 7.72 (d, J=8.0 Hz, 1H), 7.23 (d, J=8.0 Hz, 1H), 5.35 (s, 2H).

### Step 5

To a solution of compound WP08-4 (20 g, 1.0 eq.) in 200 mL of THF, compound WP08-3 (23.1 g, 1.3 eq.) and PPh₃ (34.4 g, 1.55 eq.) was added. The reaction mixture was cooled to 0° C. and DIAD (27.1 mL, 1.55 eq.) was added dropwise. The resulting mixture was then stirred overnight at room temperature. The solvent was evaporated at reduced pressure and the crude product was purified by silica gel column chromatography using 0-100% EtOAc/hexane. The desired product WP08-5 was obtained as a yellow foam (17.7 g, yield 49%).

LC/MS (ESI) m/z: 414.0.

### Step 6

To a solution of WP08-5 (14.8 g, 35.7 mmol, 1.0 eq.) in toluene (150 mL) was added n-Bu₃SnH (41.6 g, 142.9 mmol, 4.0 eq.) and AIBN (0.6 g, 3.57 mmol, 0.1 eq.). The mixture was heated to reflux and stirred overnight. TLC (PE:EA=1:1) showed no starting material remained and new spots formed. The reaction mixture was poured into saturated aq. KF solution (100 mL) and stirred overnight. Then, the reaction mixture was extracted with ethyl acetate, washed with brine, and then dried over sodium sulfate. The crude product was purified by silica gel column chromatography (DCM:MeOH=50:1) to give compound WP08-6 was obtained as a white solid (7.1 g, 60% yield).

LC/MS (ESI) m/z: 336.15.

### Step 7-8

To a solution of WP08-6 (10 g, 29.8 mmol, 1.0 eq.) in DCE (100 mL) was added α-chloroethyl chloroformate (ACE-Cl, 1.0 eq.) at 0° C. and then refluxing the mixture for 1 h. The intermediate ACE-piperidine formed and is usually de-ACEylated directly to WP08-7 by evaporating the reaction mixture in vacuo and then heating the residue in MeOH. The residue was dissolved in THF (100 mL), then 4.5 g of triethylamine (44.7 mmol, 1.5 eq.) and Boc₂O (38.7 mmol, 1.3 eq.) was added. The mixture was continued to stir for 3 h at room temperature. The solvent was evaporated under reduced pressure, then water was added, and the mixture was extracted with EA. The combined organic phases were dried over Na₂SO₄ and evaporated. The residue was purified by flash chromatograph to afford 6.0 g of product WP08-8 (2 steps, yield 60%).

LC/MS (ESI) m/z: 346.16.

### Step 9

To a solution of compound WP08-8 (15 g, 1 eq.) in tetrahydrofuran (100 mL) and water (100 mL) was added sodium hydroxide (8.7 g, 5 eq). The mixture was stirred at 20° C. for 16 h. TLC (ethyl acetate:hexane=1:1) showed reaction was complete. The mixture was adjusted to pH=5-6 with aq. hydrochloric acid (1 M) and extracted with ethyl acetate. The organic layer was washed with brine and dried over sodium sulfate. The crude material was not further purified and used as crude for the next step.

LC/MS (ESI) m/z: 364.17.

### Step 10

To a solution of compound WP08-9 (15 g, crude, 1 eq.) in dichloromethane (300 mL) was added manganese dioxide (20 eq.). The mixture was stirred at 20° C. for about 1 h. TLC showed reaction was complete. The mixture was diluted with dichloromethane and filtered through a pad of Celite. The filtrate was concentrated in vacuum. The crude product was purified by silica gel column chromatography (DCM:MeOH=10:1). The desired compound WP08-10 was obtained as yellow solid. (8 g, 2 steps, 60%).

LC/MS (ESI) m/z: 362.15.

### Step 11

To a mixture of compound WP08-10 (3 g, 1.0 eq.) in methanol (20 mL) and dichloromethane (20 mL) was added 3-aminopiperidine-2,6-dione (4.0 g, 3 eq., TFA salt), AcONa (3.08 g, 6.0 eq.) and AcOH (5.1 mL, 10.0 eq.). The mixture was stirred at 25° C. for 2 h, then sodium cyanoborohydride (1.57 g, 3.0 eq.) was added and the mixture was further stirred for 30 min. LCMS showed the reaction was complete. Next, the reaction mixture was quenched with water and concentrated under reduced pressure to give a residue which was redissolved in acetonitrile and water (1:1, 30 mL). The solution was mixed well at beginning. After standing at 0-5° C. overnight, the mixture was filtered, and the filter cake was washed with acetonitrile and water (1:1) and vacuum dried to afford the crude product WP08-11 as a solid (900 mg, yield 60%).

LC/MS (ESI) m/z: 474.22.

### Step 12

To a solution of compound WP08-11 (900 mg 1.0 equiv) in DMF (15 mL) was added HATU (795 mg, 1.1 equiv) and DIPEA (0.72 mL, 3.0 equiv), and the reaction was stirred at rt for 30 min. UPLC-MS indicated a new main peak with desired MS formed, then quenched with water and the mixture was extracted with ethyl acetate, washed with brine, and then dried over sodium sulfate. The target compound WP08-12 was obtained as a brown solid (675 mg, 75% yield).

LC/MS (ESI) m/z: 456.21. ¹H NMR (400 MHz, Chloroform-d) δ 8.00 (s, 1H), 7.50 (d, J=7.7 Hz, 1H), 7.28 (s, 1H), 5.23 (dd, J=13.3, 5.1 Hz, 1H), 4.55 (d, J=1.4 Hz, 2H), 4.46 (d, J=16.0 Hz, 1H), 4.32 (d, J=16.0 Hz, 1H), 4.15 (s, 2H), 3.01-2.77 (m, 4H), 2.38 (dd, J=13.1, 5.0 Hz, 1H), 2.29-2.17 (m, 1H), 1.92 (t, J=12.5 Hz, 2H), 1.83-1.72 (m, 2H), 1.52 (s, 9H).

### Step 13

Compound WP08-12 was treated with TFA in DCM at room temperature to de-protect the N-Boc group to provide the cereblon ligand C-1.

LC/MS (ESI) m/z: 355.1.

### 3-(7′-oxo-2′,3′,7′,9′-tetrahydro-8′H-spiro[piperidine-4,4′-pyrano[2,3-e]isoindol]-8′-yl)piperidine-2,6-dione (C-2)

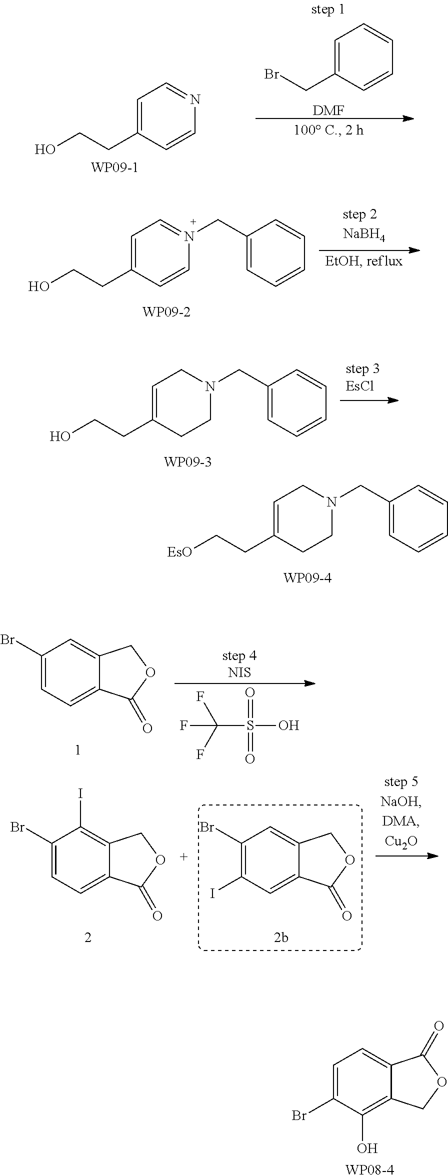

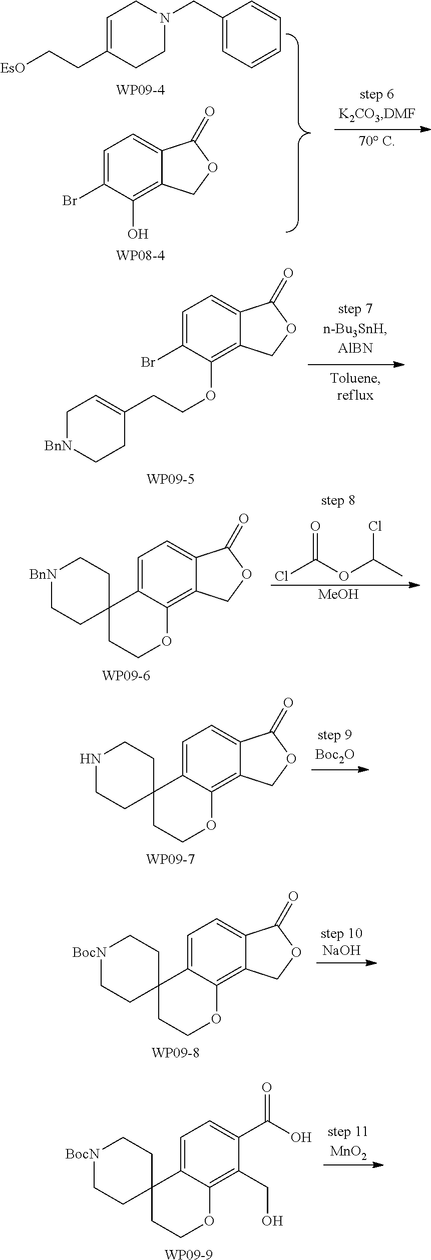

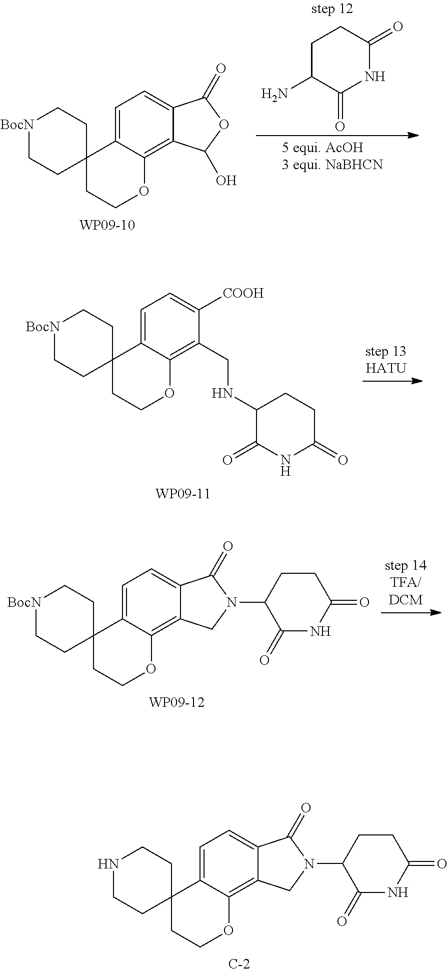

### Step 1-2

To a solution of 2-(pyridin-4-yl)ethan-1-ol (WP09-1, 10 g, 91.6 mmol, 1.0 eq.) in DMF (40 mL) was added BnBr (15.3 g, 108 mmol, 1.1 eq.). The mixture was allowed to heat to 100° C. and stirred 3 h. TLC showed no starting material remained and a new spot formed. The residue was dissolved in EtOH (150 mL), then 4.0 g of sodium borohydride (119.1 mmol, 1.3 eq.) was added portionwise at 0° C. The mixture was continued to stir at 0° C. for 1 h and then at reflux for 2 h. The solvent was evaporated under reduced pressure, then water was added, and the mixture was extracted with EA. The combined organic phases were dried over Na₂SO₄ and evaporated. The residue was purified by flash chromatograph (DCM:MeOH=100:0-30:1) to afford 10 g of product WP09-3 (Viscous oil, 2 steps, yield 56%).

LC-MS: 218 [M+H]⁺.

### Step 3

To a solution of compound WP09-3 (10 g, 1 eq.) in DCM (200.0 mL) was added DMAP (0.1 eq.) and TEA (2 eq.) at 0° C. Then EsCl (1.5 eq.) was slowly added into and the mixture was stirred at R. T. for 1 h. The reaction was partitioned between EtOAc and water. The combined organic layers were dried over anhydrous Na₂SO₄, filtered, and concentrated in vacuo. The crude residue was purified by flash chromatograph to give compound WP09-4 as a yellow solid (10 g, yield 70%).

LC-MS: 310 [M+H]⁺.

### Step 4

To a solution of 5-Bromo-3H-isobenzofuran-1-one (1) (10 g, 1 eq.) in trifluoromethanesulfonic acid (100 g, 10 V) was added NIS (12.5 g, 1.2 eq.) at 0° C. in portions. The mixture was allowed to warm to room temperature and stirred overnight. TLC showed no starting material remained and two new spots formed. The reaction mixture was poured into ice-water and yellow solid precipitated. The mixture was filtered and the filter cake was washed with ice cold water. The filter cake was dried and obtained as a yellow solid (10 g, yield 62%), to be a mixture of product 2 (top spot on TLC) and product 2b (bottom spot on TLC, which was not further reacted in next step).

### Step 5

To a mixture of compound 2 (10 g, 1 eq.), sodium hydroxide (5.75 g, 5 eq.) in water (100 mL, 1.5 M) and N,N-dimethylacetamide (60 mL) was added cuprous oxide (0.85 g, 0.2 eq.). The reaction mixture was heated to 80° C. and stirred for 12 h. TLC showed the compound 2 (top spot on TLC) was completely consumed. The reaction mixture was poured into water (100 mL) and treated with solid K₂CO₃ until pH 8-9, and extracted with EA. The aqueous layer neutralized using 1 N hydrochloride solution and extracted with ethyl acetate, washed with brine, and then dried over sodium sulfate and evaporated. The crude product was purified by silica gel column chromatography to give compound WP08-4 was obtained as a yellow solid (4.2 g, 39% yield).

LC-MS: 229/231 [M+H]⁺.

### Step 6

To a solution of compound WP08-4 (10 g, 1.0 eq.) in 100 mL of DMF, compound WP09-4 (16.2 g, 1.2 eq.) and K₂CO₃ (1.6 eq.) was added. The reaction mixture was heated to 70° C. and stirred overnight. The reaction mixture was poured into ice-water and extracted with ethyl acetate, washed with brine, and then dried over sodium sulfate. The solvent was evaporated at reduced pressure and the crude product was purified by silica gel column chromatography using 0-100% EtOAc/hexane. The desired product WP09-5 was obtained as a yellow foam (11 g, yield 60%).

LC-MS: 428/430 [M+H]⁺.

### Step 7

To a solution of WP09-5 (5 g, 1.0 eq.) in toluene (50 mL) was added n-Bu₃SnH (13.6 g, 4.0 eq.) and AIBN (0.4 g, 0.1 eq.). The mixture was heated to reflux and stirred overnight. TLC (PE:EA=1:1) showed no starting material remained and new spots formed. The reaction mixture was poured into saturated aq. KF solution (100 mL) and stirred overnight. Then, the reaction mixture was extracted with ethyl acetate, washed with brine, and then dried over sodium sulfate. The crude product was purified by silica gel column chromatography (DCM:MeOH=50:1) to give compound WP09-6 was obtained as a white solid (2 g, 50% yield).

LC-MS: 350 [M+H]⁺.

### Step 8-9

To a solution of WP09-6 (3.0 g, 1.0 eq.) in DCE (100 mL) was added α-chloroethyl chloroformate (ACE-Cl, 1.2 eq.) at 0° C. and then refluxing the mixture for 15 h. The intermediate ACE-piperidine formed and is usually deACEylated directly to WP09-7 by evaporating the reaction mixture in vacuo and then heating the residue in MeOH. The residue was dissolved in THF (100 mL), then trimethylamine (3.0 eq.) and Boc₂O (1.3 eq.) was added. The mixture was continued to stir for 3 h at room temperature. The solvent was evaporated under reduced pressure, then water was added, and the mixture was extracted with EA. The combined organic phases were dried over Na₂SO₄ and evaporated. The residue was purified by flash chromatograph to afford WP09-8 (1.5 g, 2 steps, yield 50%).

LC-MS: 360 [M+H]⁺. ¹H NMR (600 MHz, Chloroform-d) δ 7.47 (d, J=7.6 Hz, 1H), 7.08 (d, J=7.6 Hz, 1H), 5.24 (s, 2H), 4.16 (t, J=6.7 Hz, 2H), 3.88 (m, 2H), 3.51 (m, 2H), 2.52 (t, J=6.8 Hz, 2H), 2.13 (m, 2H), 1.61 (m, 2H), 1.46 (s, 9H).

### Step 10

To a solution of compound WP09-8 (2 g, 1 eq.) in tetrahydrofuran (10 mL) and water (10 mL) was added sodium hydroxide (1.2 g, 5 eq.). The mixture was stirred at 20° C. for 16 h. TLC (ethyl acetate:hexane=1:1) showed reaction was complete. The mixture was adjusted to pH=5-6 with aq. hydrochloric acid (1 M) and extracted with ethyl acetate. The organic layer was washed with brine and dried over sodium sulfate. The crude material was not further purified and used as crude for the next step.

### Step 11

To a solution of compound WP09-9 (2 g, crude, 1 eq.) in dichloromethane (30 mL) was added manganese dioxide (20 eq.). The mixture was stirred at 20° C. for about 1 h. TLC showed reaction was complete. The mixture was diluted with dichloromethane and filtered through a pad of Celite. The filtrate was concentrated in vacuum. The crude product was purified by silica gel column chromatography (DCM:MeOH=10:1). The desired compound WP09-10 was obtained as yellow solid. (1.2 g, 2 steps, 60%).

LC-MS: 376 [M+H]⁺.

### Step 12

To a mixture of compound WP09-10 (532 mg, 1.0 eq.) in methanol (5 mL) and dichloromethane (5 mL) was added 3-aminopiperidine-2,6-dione (698 mg, 3 eq., HCl salt), AcONa (698 mg, 6.0 eq.) and AcOH (0.85 mL, 10.0 eq.). The mixture was stirred at 25° C. for 1 h, then sodium cyanoborohydride (268 mg, 3.0 eq.) was added and the mixture was further stirred for 30 min. LCMS showed the reaction was complete. Next, the reaction mixture was quenched with water and concentrated under reduced pressure to give a residue which was purified by pre-HPLC (20%˜50% ACN, neutral). The desired product WP09-11 as a solid (415 mg, yield 60%) after lyophilization.

### Step 13

To a solution of compound WP09-11 (415 mg 1.0 equiv) in DMF (5 mL) was added HATU (421 mg, 1.3 equiv) and DIPEA (0.47 mL, 3.0 equiv), and the reaction was stirred at rt for 30 min. UPLC-MS indicated a new main peak with desired MS formed, then quenched with water and the mixture was extracted with ethyl acetate, washed with brine, and then dried over sodium sulfate. The desired compound WP09-12 was obtained as a brown solid (300 mg, 75% yield).

LC-MS: 470 [M+H]⁺.

### Step 14

Compound WP09-12 was treated with TFA in DCM at room temperature to de-protect the N-Boc group to provide the cereblon ligand C-2.

LC/MS (ESI) m/z: 369.2.

### (S)-3-(6′-oxo-1′,2′,6′,8′-tetrahydro-7′H-spiro[piperidine-4,3′-pyrrolo[3,4-g]indol]-7′-yl)piperidine-2,6-dione (C-3); and

### (S)-3-(1′-methyl-6′-oxo-1′,2′,6′,8′-tetrahydro-7′H-spiro[piperidine-4,3′-pyrrolo[3,4-g]indol]-7′-yl)piperidine-2,6-dione (C-5)

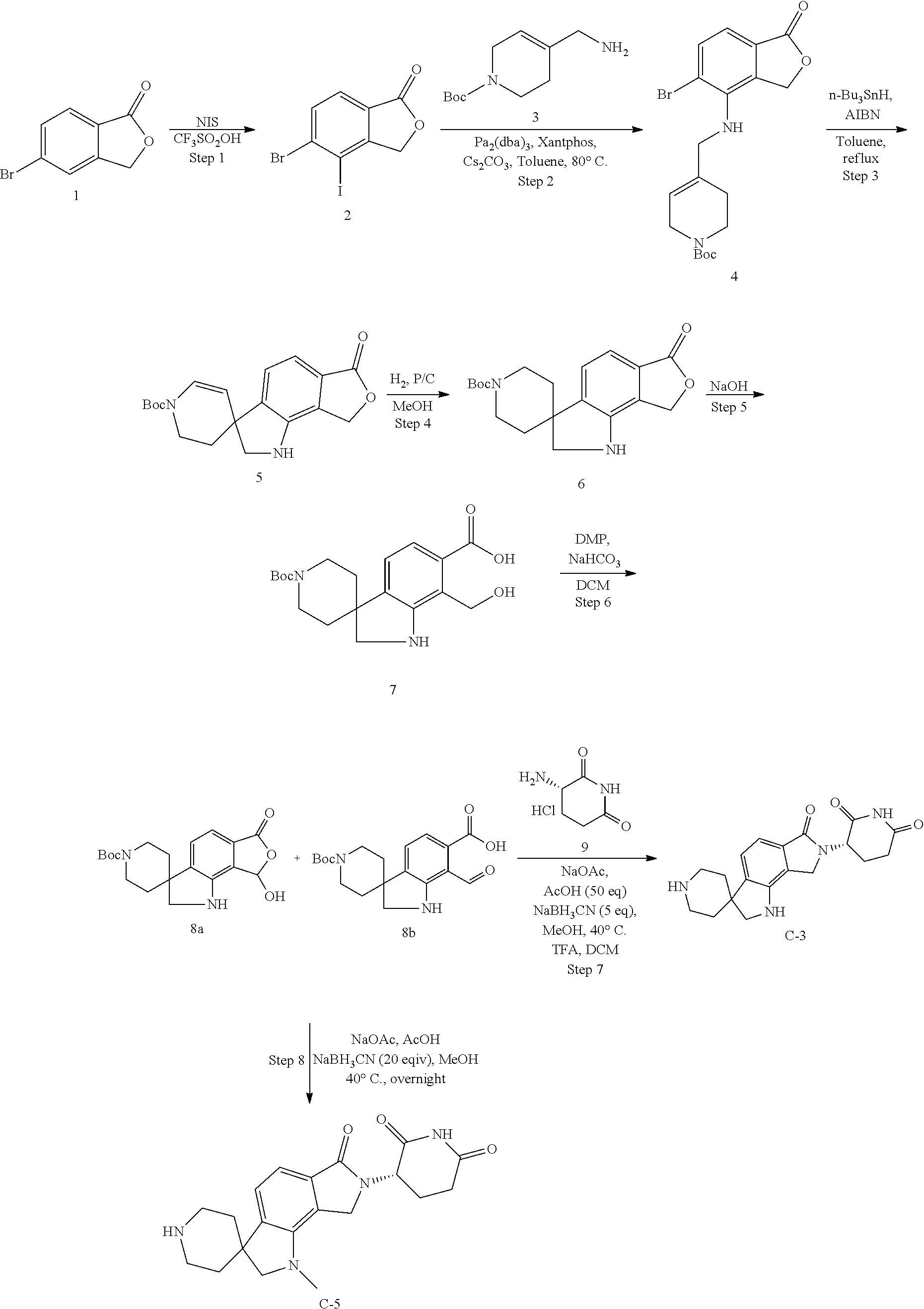

### Step 1

To a solution of 5-Bromo-3H-isobenzofuran-1-one (1) (10 g, 1 eq.) in trifluoromethanesulfonic acid (100 g, 10 V) was added NIS (12.5 g, 1.2 eq.) at 0° C. in portions. The mixture was allowed to warm to room temperature and stirred overnight. TLC showed no starting material remained and two new spots formed. The reaction mixture was poured into ice-water and yellow solid precipitated. The mixture was filtered and the filter cake was washed with ice cold water. The filter cake was dried and obtained as a yellow solid (10 g, yield 62%), to be a mixture of product 2 (top spot on TLC) and product 2b (bottom spot on TLC, which was not further reacted in next step).

### Step 2

Compound 3 was made according to the procedure reported (Bioorg. Med. Chem. Lett. 2016, 26, 228-234). To a flask containing compound 2 (500 mg, 1.0 eq.), compound 3 (377 mg, 1.2 eq.), Pd₂(dba)₃ (136 mg, 0.1 eq.), Xantphos (257 mg, 0.3 eq.) and Cs₂CO₃ (1447 mg, 3.0 eq.) was added Toluene (15 mL). The reaction was evacuated and backfilled with N₂ three times. The reaction was stirred at 80° C. for 6 h and then was allowed to cool to room temperature and filtered. The filtrate was evaporated, and the residue was purified by silica gel chromatography (0-25% ethyl acetate in hexane) to afford product 4 as a light yellow powder 316 mg (yiled=51%).

LC-MS: 323.14 [M+H]+. 1H NMR (400 MHz, Chloroform-d) δ 7.61 (d, J=8.0 Hz, 1H), 7.17 (d, J=7.9 Hz, 1H), 5.64-5.53 (m, 1H), 5.36 (s, 2H), 3.93-3.79 (m, 4H), 3.53 (t, J=5.7 Hz, 2H), 2.17-2.05 (m, 2H), 1.46 (s, 9H).

### Step 3 and Step 4

To a solution of compound 4 (300 mg, 1.0 eq.) and AIBN (35 mg, 0.3 eq.) in Toluene (10 mL) was added Bu₃SnH (954 uL, 5.0 eq.). The reaction was stirred at 110° C. in a sealed tube for 24 h. Then cooled to room temperature, quenched with saturated aq. KF solution (20 mL) and kept the mixture stirring overnight. The result mixture was extracted with ethyl acetate (3 times). The combined organic layers were washed with brine (3 times), dried over Na₂SO₄, filtered, and concentrated under reduced pressure to give a crude mixture. The mixture was purified by silica gel chromatography (0-30% ethyl acetate in hexane) to afford the crude product 5 as a light-yellow oil (90 mg).

LC-MS: 343.37 [M+H]⁺.

To a solution of compound 5 (90 mg) in MeOH (5 mL) was added Pd/C (90 mg). The reaction was evacuated and backfilled with H₂ and stirred at room temperature under H₂ atmosphere for 6 h. Then filtered through celite and the filtration was concentrated under reduced pressure to give the cude product, which is purified by silica gel chromatography (0-50% ethyl acetate in hexane) to afford the compound 6 as a white solid (50 mg, 20% yield for steps 3 and 4).

LC-MS: 345.22 [M+H]⁺. ¹H NMR (400 MHz, Chloroform-d) δ 7.37 (d, J=7.6 Hz, 1H), 7.19 (d, J=7.6 Hz, 1H), 5.20 (s, 2H), 4.19-4.04 (m, 2H), 3.67 (s, 2H), 3.00-2.77 (m, 2H), 1.91-1.68 (m, 4H), 1.48 (s, 9H). ¹³C NMR (101 MHz, CDCl₃) δ 171.42, 154.87, 143.02, 142.81, 128.22, 126.10, 123.66, 117.49, 79.98, 67.97, 56.18, 44.96, 40.85, 35.64, 28.54.

### Step 5

To a solution of 6 (48 mg, 1.0 equiv) in THF/MeOH/H₂O (2 mL/2 ml/1 mL) was added NaOH (111 mg, 20 equiv). The reaction was stirred at rt overnight, then concentrated to remove most of the THF/MeOH. The residue was diluted with 1 mL water, followed by neutralization with 2 N aq HCl to PH 4-6, then extracted with EA (5 mL, 6 times). The combined organic layer was washed with brine, filtered, dried with Na₂SO₄, and concentrated under reduced pressure to give the crude product 7 as a light-yellow oil 50 mg, which was directly used in the next step.

LC-MS: 363.28 [M+H]⁺.

### Step 6

To a solution of 7 (40 mg, 1.0 equiv) in DCM (5 mL) was added NaHCO₃ (28 mg, 3.0 eq.), followed by add DMP (47 mg, 1.0 equiv) potionwise. 10 min Later, the reaction mixture was diluted with DCM and washed with brine, dried over Na₂SO₄, filtered, and concentrated to afford the crude product 8 as a yellow oil 40 mg, which was directly used in the next step.

LC-MS: 361.27 [M+H]⁺.

### Step 7

To a solution of 9 (73 mg, 4.0 equiv) and NaOAc (28 mg, 4 equiv) in MeOH (4 mL) was added 8 (40 mg, 1.0 equiv) and AcOH (317 uL, 50 eq.). 15 min Later, NaBH₃CN (34.5 mg, 5.0 eq.) was added, and the resulted mixture was stirred at 40° C. for 3 h. The reaction mixture was concentrated to remove some MeOH, and then purified by pre-HPLC to give the Boc-protected C-3, which is further treated with TFA and concentrated to remove TFA. The final compound C-3 was obtained as a white solid 10 mg.

LC-MS: 423.16 [M+H]⁺. ¹H NMR (400 MHz, Methanol-d₄) δ 7.28-7.17 (m, 2H), 5.14 (dd, J=13.3, 5.2 Hz, 1H), 4.39-4.23 (m, 2H), 3.67 (s, 2H), 3.51-3.39 (m, 2H), 3.25-3.12 (m, 2H), 2.98-2.84 (m, 1H), 2.84-2.72 (m, 1H), 2.56-2.39 (m, 1H), 2.23-1.96 (m, 5H).

### Step 8

To a solution of 9 (18 mg, 4.0 equiv) and NaOAc (6.9 mg, 4 equiv) in MeOH (3 mL) was added 8 (40 mg, 1.0 equiv) and AcOH (0.5 mL). 15 min Later, NaBH₃CN (34.5 mg, 20 eq.) was added in potionwise, and the resulted mixture was stirred at 40° C. for overnight. The reaction mixture was concentrated to remove some MeOH, and then purified by pre-HPLC to give the Boc-protected C-5, which is further treated with TFA and concentrated to remove TFA. The final compound C-5 was obtained as a gray solid 4.7 mg.

LC-MS: 469.26 [M+H]⁺. ¹H NMR (400 MHz, Methanol-d₄) δ 7.21 (s, 2H), 5.14 (dd, J=13.3, 5.2 Hz, 1H), 4.72-4.55 (m, 2H), 3.51-3.39 (m, 4H), 3.24-3.13 (m, 2H), 2.99 (s, 3H), 2.96-2.83 (m, 1H), 2.83-2.74 (m, 1H), 2.60-2.46 (m, 1H), 2.22-2.03 (m, 3H), 2.03-1.94 (m, 2H).

### 3-(6-oxo-6,8-dihydrospiro[furo[3,4-e]isoindole-3,4′-piperidin]-7(1H)-yl)piperidine-2,6-dione (C7)

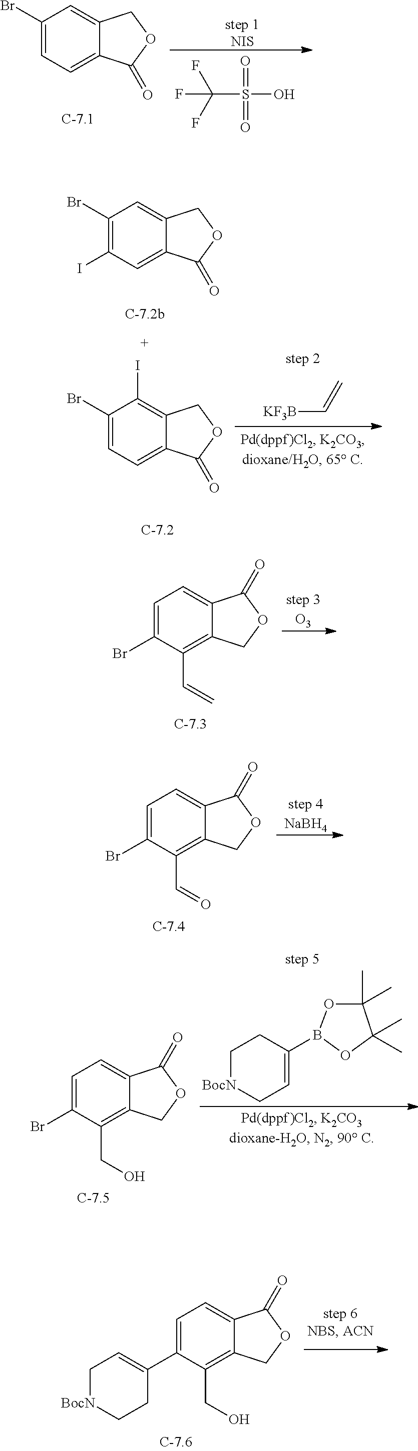

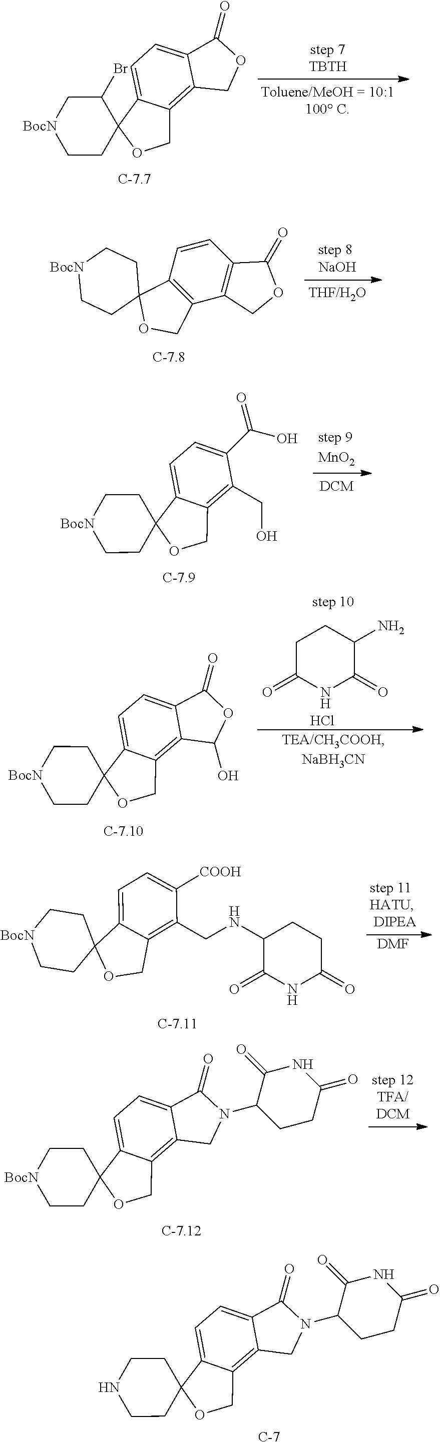

### Step 1

To a solution of 5-Bromo-3H-isobenzofuran-1-one (C-7.1) (10 g, 1 eq.) in trifluoromethanesulfonic acid (80 mL, 20 eq.) was added NIS (12.5 g, 1.2 eq.) at 0° C. in portions. The mixture was allowed to warm to room temperature and stirred overnight. TLC showed no starting material remained and two new spots formed. The reaction mixture was poured into ice-water and yellow solid precipitated. The mixture was filtered and the filter cake was washed with ice cold water. The filter cake was dissolved in DCM and dried over sodium sulfate. The mixture was filtered and the filtrate was concentrated to afford a yellow solid. The crude product was purified by silica gel flash chromatography. The less polar product (top spot on TLC) C-7.2 was obtained as a brown solid (8 g, yield 50%).

### Step 2

A vial was charged with compound C-7.2 (8 g, 1 eq.), Pd(dppf)Cl₂ (0.2 eq.), K₂CO₃ (3 eq.) and dioxane-H₂O (100 mL/20 mL). The mixture was purged with nitrogen and potassium vinyltrifluoroborate (2.0 eq.) was added into. The reaction was heated to 65° C. for 16 h. TLC showed reaction was complete. The mixture was diluted with ethyl acetate and washed with water. The organic layer was washed with brine and dried over sodium sulfate. The crude product was purified by silica gel column chromatography using 0-50% EtOAc/hexane to give compound C-7.3 as a yellow foam (3.2 g, yield 57%).

LC-MS: 239/241 [M+H]⁺; ¹H NMR (600 MHz, Chloroform-d) δ 7.70 (d, J=8.1 Hz, 1H), 7.58 (d, J=8.1 Hz, 1H), 6.92 (dd, J=18.0, 11.6 Hz, 1H), 5.62 (d, J=11.6 Hz, 1H), 5.42 (d, J=18.0 Hz, 1H), 5.33 (s, 2H).

### Step 3

A solution of compound C-7.3 (5 g) in CH₂Cl₂ (100 mL) was cooled to −78° C. then O₃ was bubbled into this solution. The passage of O₃ was continued for a further 30 min until the color turned pale blue and then air was bubbled into the solution for 10 min to remove excess O₃. After dropwise addition of Me₂S (2 mL), the solution was kept stirred and warmed to room temperature. The mixture was diluted with water and extracted with DCM. The organic layer was washed with brine and dried over MgSO₄. The residue was quickly purified by chromatography to give compound C-7.4 (4 g).

### Step 4

To a solution of compound C-7.4 (4 g, 1.0 eq.) in MeOH (40 mL, 10V) was added NaBH₄ (1.9 g, 3 eq.) at 0° C. in portions. TLC indicated compound 4 was consumed completely and LCMS indicated there was desired product. The reaction mixture was quenched by addition H₂O at 20° C., and then concentrated under reduced pressure to remove MeOH. Then the mixture was extracted with EtOAc). The combined organic layers were washed with brine, dried over Na₂SO₄, filtered and concentrated under reduced pressure. The residue was purified by column chromatography (SiO₂, Petroleum ether/Ethyl acetate=1/1 to Ethyl acetate) to give compound C-7.5 (3 g, yield 75%).

### Step 5

A round bottomed flask equipped with a stirrer bar was charged with a mixture of compound C-7.5 (4 g, 1.0 eq.), tert-butyl 4-(4,4,5,5-tetramethyl-1,3,2-dioxaborolan-2-yl)-3,6-dihydropyridine-1(2H)-carboxylate (7.7 g, 1.5 eq.), potassium carbonate (6.9 g, 3.0 eq.), and Pd(dppf)Cl₂ (2.4 g, 0.2 eq.). The flask was evacuated and back-filled with nitrogen (×3). The mixture of dioxane-H₂O (100 mL/20 mL) was added and kept stirred at 90° C. for 10 hours. The cooled reaction mixture was diluted with EtOAc and filtered through Celite™ to remove insoluble material. The filtrate was washed with water, saturated aqueous sodium chloride and then dried over magnesium sulfate, filtered and the filtrate concentrated. The crude material was purified by flash silica chromatography, elution gradient MeOH in DCM. Pure fractions were combined and concentrated to afford compound C-7.6 (5 g, 89%).

### Step 6

To a mixture of compound C-7.6 (6 g, 1 eq.) in MeCN (60 mL) was added NBS (3.7, 1.2 eq.) in one portion. The mixture was stirred at 20° C. for 16 h. The mixture was concentrated in vacuum and the crude material was purified by flash silica chromatography, elution gradient MeOH in DCM to give compound C-7.7 was obtained as a white solid (6.6 g, 90% yield).

¹H NMR (600 MHz, Chloroform-d) δ 7.87 (d, J=7.9 Hz, 1H), 7.66 (d, J=7.9 Hz, 1H), 5.25 (s, 2H), 5.16 (d, J=13.0 Hz, 1H), 5.05 (d, J=13.0 Hz, 1H), 4.25 (m, 2H), 4.08-3.81 (m, 2H), 3.33 (m, 1H), 2.68-2.56 (m, 1H), 1.65 (d, J=13.9 Hz, 1H), 1.50 (s, 9H).

### Step 7

To a solution of compound C-7.7 (500 mg, 1.0 eq.) in toluene (10 mL) and MeOH (1 mL) was added n-Bu₃SnH (5.0 eq.) and AIBN (0.1 eq.). The mixture was heated to reflux and stirred overnight. After cooling down, an additional n-Bu₃SnH (5.0 eq.) was added into above mixture and kept stirred at 100° C. for another 12 h. TLC showed no starting material remained and the reaction mixture was poured into saturated aq. KF solution (100 mL) and stirred for 1 h. Then, the reaction mixture was filtered and extracted with ethyl acetate, washed with brine, and then dried over sodium sulfate. The crude product was purified by silica gel column chromatography (PE:EA=4:1) to give compound C-7.8 was obtained as a white solid (60% yield). LC-MS: 346 [M+H]⁺; ¹H NMR (600 MHz, DMSO-d₆) δ 7.78 (d, J=7.8 Hz, 1H), 7.57 (d, J=7.8 Hz, 1H), 5.39 (s, 2H), 5.09 (s, 2H), 3.98 (brs, 2H), 3.07 (brs, 2H), 1.88 (td, J=13.1, 4.9 Hz, 2H), 1.64 (dd, J=13.8, 2.4 Hz, 2H), 1.43 (s, 9H).

### Step 8

To a solution of compound C-7.8 (1.25 g, 1 eq.) in tetrahydrofuran (10 mL) and water (10 mL) was added sodium hydroxide (720 mg, 5 eq.). The mixture was stirred at 20° C. for 16 h. TLC (ethyl acetate:hexane=1:1) showed reaction was complete. The mixture was adjusted to pH=5-6 with aq. hydrochloric acid (1 M) and extracted with ethyl acetate. The organic layer was washed with brine and dried over sodium sulfate. The crude material 9 was not further purified and used as crude for the next step.

### Step 9

To a solution of compound C-7.9 (1 g, crude, 1 eq.) in dichloromethane (50 mL) was added manganese dioxide (20 eq.). The mixture was stirred at 20° C. for about 1 h. TLC showed reaction was complete. The mixture was diluted with dichloromethane and MeOH, then filtered through a pad of Celite. The filtrate was concentrated in vacuum and the crude product C-7.10 (0.6 g, crude) was used directly in the next step.

### Step 10

To a mixture of compound C-7.10 (300 mg, crude, 1.0 eq.) in methanol (5 mL) and dichloromethane (5 mL) was added 3-aminopiperidine-2,6-dione (162 mg, 1.5 eq., HCl salt), AcONa (204 mg, 3.0 eq.) and AcOH (150 μL, 3.0 eq.). The mixture was stirred at 20° C. for 1 h, then sodium cyanoborohydride (104 mg, 2.0 eq.) was added and the mixture was further stirred for 30 min. LCMS showed the reaction was complete. Next, the reaction mixture was concentrated under reduced pressure to give a residue which was purified by pre-HPLC (20%˜50% ACN, neutral). The desired product C-7.11 was obtained as a white solid 120 mg after lyophilization.

### Step 11

To a solution of compound C-7.11 (180 mg 1.0 equiv) in DMF (3 mL) was added HATU (216 mg, 1.5 equiv) and DIPEA (0.2 mL, 3.0 equiv) at 0° C., and the reaction was stirred at rt for 30 min. UPLC-MS indicated a new main peak with desired MS formed, then quenched with water and the mixture was extracted with ethyl acetate, washed with brine, and then dried over sodium sulfate. The target compound C-7.12 was obtained as a brown solid (100 mg, 60% yield).

LC-MS: 456 [M+H]⁺; ¹H NMR (400 MHz, DMSO-d6) δ 11.01 (s, 1H), 7.66 (d, J=7.7 Hz, 1H), 7.47 (d, J=7.7 Hz, 1H), 5.16-5.11 (m, 1H), 5.08 (m, 2H), 4.44 (d, J=17.4 Hz, 1H), 4.29 (d, J=17.4 Hz, 1H), 3.98 (m, 2H), 3.04 (m, 3H), 2.60 (d, J=17.0 Hz, 1H), 2.42-2.31 (m, 1H), 2.04-1.93 (m, 1H), 1.92-1.80 (m, 2H), 1.64 (m, 2H), 1.43 (s, 9H).

### Step 12

Compound C-7.12 was treated with TFA in DCM at room temperature to de-protect the N-Boc group to provide the cereblon ligand C-7.

LC/MS (ESI) m/z: 355.1.

### Synthetic Procedures for Preparing IKZF2 Degraders 3-362

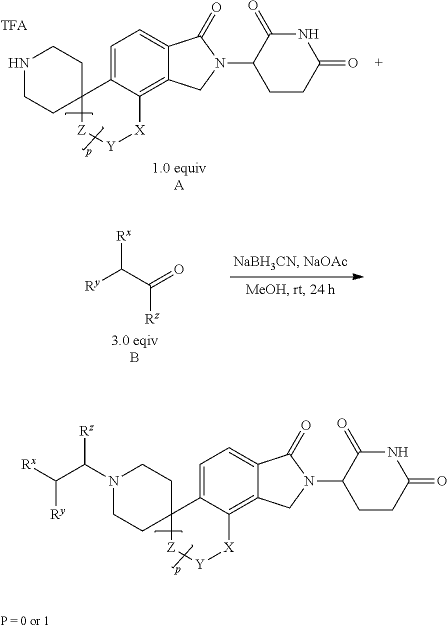

To a solution of A (0.02 mmol) in MeOH (4 mL) was added NaOAc (0.06 mmol), followed by B (0.06 mmol). Ater being stirred at rt for 30 m, 5 equivalent NaBH₃CN (0.10 mmol) was added. 12 h later, additional 5 equivalent NaBH₃CN (0.1 mmol) was added. The result reaction mixture was kept stirred for additional 12 h. Then the solvent was removed under reduced pressure, and the result residue was purified by pre-HPLC to obtain the title compounds 3-362.

### II. Characterization of Compounds

Characterizational Data of the compounds is shown in Table E2.

TABLE E1
Characterization Data

| | LC-MS: | |
|---|---|---|
| No. | [M + H]⁺ | ¹H-NMR |
| A1 | 356.15 | ¹H NMR (400 MHz, CDCl₃) δ 8.00 (s, 1H), 7.50 (d, |
| | | J = 7.7 Hz, 1H), 7.28 (s, 1H), 5.23 (dd, J = 13.3, |
| | | 5.1 Hz, 1H), 4.55 (d, J = 1.4 Hz, 2H), 4.46 (d, J = |
| | | 16.0 Hz, 1H), 4.32 (d, J = 16.0 Hz, 1H), 4.15 (s, |
| | | 2H), 3.01-2.77 (m, 4H), 2.38 (dd, J = 13.1, 5.0 |
| | | Hz, 1H), 2.29-2.17 (m, 1H), 1.92 (t, J = 12.5 Hz, |
| | | 2H), 1.83-1.72 (m, 2H). |
| A2 | 370.17 | ¹H NMR (400 MHz, CDCl₃) δ 8.03 (s, 1H), 7.45 (d, |
| | | J = 1.2 Hz, 2H), 5.22 (dd, J = 13.2, 5.1 Hz, 1H), |
| | | 4.40 (d, J = 16.4 Hz, 1H), 4.31-4.20 (m, 3H), 4.11 |
| | | (brs, 1H), 3.06-2.78 (m, 4H), 2.37 (qd, J = 13.1, |
| | | 5.0 Hz, 1H), 2.22 (dtd, J = 13.1, 5.3, 2.7 Hz, 1H), |
| | | 2.17-2.04 (m, 3H), 1.60-1.50 (s, 4H). |
| A3 | 496.16 | ¹H NMR (400 MHz, Methanol-d₄) δ 7.73 (t, J = 6.4 |
| | | Hz, 4H), 7.44 (d, J = 7.6 Hz, 1H), 7.33 (d, J = 7.8 |
| | | Hz, 1H), 6.87 (t, J = 55.9 Hz, 1H), 5.15 (dd, J = |
| | | 13.3, 5.2 Hz, 1H), 4.73 (s, 2H), 4.45 (d, J = 12.3 |
| | | Hz, 3H), 3.59 (d, J = 12.8 Hz, 2H), 3.41-3.36 (m, |
| | | 1H), 3.22 (d, J = 13.1 Hz, 2H), 2.92 (ddd, J = 18.5, |
| | | 13.5, 5.4 Hz, 1H), 2.80 (ddd, J = 17.6, 4.7, 2.4 Hz, |
| | | 1H), 2.52 (qd, J = 13.3, 4.8 Hz, 1H), 2.34-2.01 |
| | | (m, 5H). |
| A4 | 498.15 | ¹H NMR (400 MHz, Methanol-d₄) δ 7.64 (t, J = 8.1 |
| | | Hz, 1H), 7.52-7.25 (m, 4H), 5.15 (dd, J = 13.4, |
| | | 5.1 Hz, 1H), 4.71 (s, 2H), 4.47 (dd, J = 17.3, 12.2 |
| | | Hz, 3H), 3.62 (d, J = 12.8 Hz, 2H), 3.31-3.09 (m, |
| | | 2H), 2.92 (ddd, J = 18.4, 13.4, 5.4 Hz, 1H), 2.80 |
| | | (ddd, J = 17.6, 4.7, 2.4 Hz, 1H), 2.52 (qd, J = 13.2, |
| | | 4.7 Hz, 1H), 2.38-2.01 (m, 5H). |
| A5 | 482.21 | ¹H NMR (400 MHz, Methanol-d₄) δ 7.62-7.28 |
| | | (m, 5H), 5.15 (dd, J = 13.3, 5.1 Hz, 1H), 4.72 (s, |
| | | 2H), 4.50-4.32 (m, 3H), 3.59 (d, J = 12.8 Hz, 2H), |
| | | 3.41-3.36 (m, 1H), 3.31-3.09 (m, 2H), 2.92 |
| | | (ddd, J = 17.6, 13.5, 5.4 Hz, 1H), 2.80 (ddd, J = |
| | | 17.5, 4.7, 2.4 Hz, 1H), 2.52 (qd, J = 13.2, 4.7 Hz, |
| | | 1H), 2.34-2.04 (m, 5H). |
| A6 | 510.21 | ¹H NMR (400 MHz, Methanol-d₄) δ 7.76 (dd, J = |
| | | 8.7, 5.9 Hz, 1H), 7.50 (dd, J = 8.6, 2.7 Hz, 1H), |
| | | 7.44 (d, J = 7.6 Hz, 1H), 7.31 (td, J = 8.3, 2.6 Hz, |
| | | 2H), 5.16 (dd, J = 13.3, 5.2 Hz, 1H), 4.74 (s, 2H), |
| | | 4.57 (s, 2H), 4.53-4.38 (m, 2H), 3.65 (d, J = 12.8 |
| | | Hz, 2H), 3.41-3.36 (m, 2H), 2.92 (ddd, J = 17.6, |
| | | 13.4, 5.4 Hz, 1H), 2.80 (ddd, J = 17.6, 4.7, 2.5 Hz, |
| | | 1H), 2.52 (qd, J = 13.2, 4.7 Hz, 1H), 2.30 (t, J = |
| | | 14.1 Hz, 2H), 2.23-2.04 (m, 3H). |
| A7 | 452.25 | |
| A8 | 446.20 | ¹H NMR (400 MHz, Methanol-d₄) δ 7.55 (t, J = 2.4 |
| | | Hz, 5H), 7.43 (d, J = 7.7 Hz, 1H), 7.34 (t, J = 7.0 |
| | | Hz, 1H), 5.15 (dd, J = 13.4, 5.2 Hz, 1H), 4.73 (s, |
| | | 2H), 4.45 (d, J = 12.3 Hz, 2H), 4.40 (s, 2H), 3.59 |
| | | (d, J = 12.9 Hz, 2H), 3.21 (t, J = 13.0 Hz, 2H), 2.92 |
| | | (ddd, J = 17.6, 13.5, 5.4 Hz, 1H), 2.80 (ddd, J = |
| | | 17.6, 4.7, 2.4 Hz, 1H), 2.52 (qd, J = 13.2, 4.7 Hz, |
| | | 1H), 2.31-2.14 (m, 3H), 2.10 (d, J = 15.0 Hz, 2H). |
| A9 | 510.21 | ¹H NMR (400 MHz, Methanol-d₄) δ 7.72 (s, 4H), |
| | | 7.56-7.46 (m, 1H), 7.37 (d, J = 7.9 Hz, 1H), 6.86 |
| | | (t, J = 55.9 Hz, 1H), 5.13 (dd, J = 13.3, 5.1 Hz, |
| | | 1H), 4.62-4.25 (m, 6H), 3.48 (d, J = 12.8 Hz, 2H), |
| | | 3.42-3.34 (m, 2H), 2.91 (ddd, J = 17.6, 13.4, 5.3 |
| | | Hz, 1H), 2.79 (ddd, J = 17.6, 4.7, 2.4 Hz, 1H), 2.60- |
| | | 2.32 (m, 3H), 2.30-2.10 (m, 3H), 2.05-1.79 |
| | | (m, 2H). |
| A10 | 512.17 | ¹H NMR (400 MHz, Methanol-d₄) δ 7.71-7.32 |
| | | (m, 5H), 5.14 (dt, J = 13.3, 5.0 Hz, 1H), 4.54-4.43 |
| | | (m, 2H), 4.39 (d, J = 11.6 Hz, 1H), 4.35-4.27 (m, |
| | | 2H), 3.54 (d, J = 12.2 Hz, 2H), 3.48-3.35 (m, 3H), |
| | | 2.92 (ddd, J = 18.5, 13.4, 5.3 Hz, 1H), 2.79 (ddd, J = |
| | | 17.5, 4.7, 2.4 Hz, 1H), 2.60-2.33 (m, 3H), 2.33- |
| | | 2.11 (m, 3H), 2.00 (d, J = 15.0 Hz, 2H). |
| A11 | 488.25 | ¹H NMR (400 MHz, Methanol-d₄) δ 7.60-7.25 |
| | | (m, 6H), 5.13 (dd, J = 13.3, 5.0 Hz, 1H), 4.64- |
| | | 4.15 (m, 6H), 3.47 (d, J = 13.0 Hz, 2H), 3.43-3.34 |
| | | (m, 2H), 2.91 (ddd, J = 18.4, 13.4, 5.3 Hz, 1H), |
| | | 2.85-2.63 (m, 3H), 2.62-2.46 (m, 1H), 2.39 (t, J = |
| | | 13.3 Hz, 2H), 2.30-2.11 (m, 3H), 1.97 (d, J = |
| | | 14.9 Hz, 2H), 1.37-1.14 (m, 3H). |
| A12 | 474.23 | ¹H NMR (400 MHz, Methanol-d₄) δ 7.56-7.26 |
| | | (m, 6H), 5.13 (dd, J = 13.3, 5.0 Hz, 1H), 4.52- |
| | | 4.25 (m, 6H), 3.45 (m, 2H), 3.35-3.30 (m, 2H), 2.91 |
| | | (ddd, J = 18.5, 13.4, 5.3 Hz, 1H), 2.79 (ddd, J = |
| | | 17.7, 4.8, 2.4 Hz, 1H), 2.53 (td, J = 13.2, 4.7 Hz, |
| | | 1H), 2.40 (m, 5H), 2.29-2.10 (m, 3H), 1.97 (d, J = |
| | | 14.7 Hz, 2H). |
| A13 | 460.22 | ¹H NMR (400 MHz, Methanol-d₄) δ 7.68-7.45 |
| | | (m, 6H), 7.36 (d, J = 8.0 Hz, 1H), 5.13 (dd, J = |
| | | 13.3, 5.1 Hz, 1H), 4.52-4.23 (m, 5H), 3.56-3.42 |
| | | (m, 2H), 3.33 (dq, J = 3.3, 1.8 Hz, 3H), 2.91 (ddd, J = |
| | | 17.5, 13.4, 5.3 Hz, 1H), 2.79 (ddd, J = 17.7, |
| | | 4.7, A 2.5 Hz, 1H), 2.60-2.47 (m, 1H), 2.47-2.31 |
| | | (m, 2H), 2.30-2.11 (m, 3H), 2.04-1.87 (m, 2H). |
| A14 | 466.26 | ¹H NMR (400 MHz, Methanol-d₄) δ 7.54 (dd, J = |
| | | 7.9, 5.4 Hz, 1H), 7.41 (d, J = 8.0 Hz, 1H), 5.14 (dd, |
| | | J = 13.3, 5.1 Hz, 1H), 4.50-4.28 (m, 3H), 3.58 (d, |
| | | J = 13.2 Hz, 2H), 3.29-3.19 (m, 2H), 3.07 (t, J = |
| | | 7.2 Hz, 2H), 2.92 (ddd, J = 18.6, 13.5, 5.4 Hz, 1H), |
| | | 2.80 (ddd, J = 17.5, 4.7, 2.4 Hz, 1H), 2.60-2.38 |
| | | (m, 3H), 2.29-2.11 (m, 3H), 1.88 (ddt, J = 45.6, |
| | | 31.1, 14.9 Hz, 8H), 1.49-1.20 (m, 4H), 1.20- |
| | | 0.99 (m, 2H). |
| A15 | 460.22 | ¹H NMR (400 MHz, Methanol-d₄) δ 7.44 (dd, J = |
| | | 7.8, 3.4 Hz, 3H), 7.35 (dd, J = 11.3, 7.8 Hz, 3H), |
| | | 5.15 (dd, J = 13.3, 5.1 Hz, 1H), 4.72 (s, 2H), 4.54- |
| | | 4.38 (m, 2H), 4.35 (s, 2H), 3.58 (d, J = 13.2 Hz, |
| | | 2H), 3.27-3.11 (m, 2H), 2.92 (ddd, J = 18.5, 13.4, |
| | | 5.4 Hz, 1H), 2.80 (ddd, J = 17.6, 4.7, 2.4 Hz, 1H), |
| | | 2.57 (d, J = 4.7 Hz, 1H), 2.42 (s, 3H), 2.07 (d, J = |
| | | 9.1 Hz, 5H). |
| A16 | 474.23 | ¹H NMR (400 MHz, Methanol-d₄) δ 7.55-7.27 |
| | | (m, 6H), 5.15 (dd, J = 13.3, 5.1 Hz, 1H), 4.76 (s, |
| | | 2H), 4.45 (d, J = 12.6 Hz, 2H), 4.36 (s, 2H), 3.58 |
| | | (d, J = 12.9 Hz, 2H), 3.26-3.11 (m, 2H), 2.92 |
| | | (ddd, J = 18.5, 13.5, 5.4 Hz, 1H), 2.82 (dd, J = 4.8, |
| | | 2.5 Hz, 1H), 2.80-2.65 (m, 2H), 2.52 (qd, J = |
| | | 13.3, 4.9 Hz, 1H), 2.30-2.01 (m, 5H), 1.27 (t, J = |
| | | 7.6 Hz, 3H). |
| A17 | 488.23 | |
| A18 | 438.25 | |
| A19 | 480.28 | |
| A20 | 454.25 | |
| A21 | 500.17 | |
| A22 | 486.21 | |
| A23 | 504.22 | |
| A24 | 464.26 | |
| A25 | 522.10 | |
| A26 | 478.21 | |
| A27 | 496.18 | ¹H NMR (400 MHz, Methanol-d₄) δ 8.30 (d, J = |
| | | 8.5 Hz, 1H), 8.09 (d, J = 8.3 Hz, 1H), 8.03 (d, J = |
| | | 8.0 Hz, 1H), 7.80 (d, J = 7.0 Hz, 1H), 7.77-7.70 |
| | | (m, 1H), 7.68-7.59 (m, 2H), 7.39 (d, J = 7.8 Hz, |
| | | 1H), 7.26 (d, J = 7.7 Hz, 1H), 5.13 (dd, J = 13.4, |
| | | 5.1 Hz, 1H), 4.90 (s, 2H), 4.75 (s, 2H), 4.51-4.35 |
| | | (m, 2H), 3.69-3.59 (m, 2H), 3.44-3.33 (m, 2H), |
| | | 2.96-2.84 (m, 1H), 2.82-2.72 (m, 1H), 2.57- |
| | | 2.42 (m, 1H), 2.30-2.12 (m, 3H), 2.11-2.02 (m, |
| | | 2H). |
| A28 | 482.16 | |
| A29 | 464.22 | |
| A30 | 510.15 | |
| A31 | 474.23 | |
| A32 | 480.17 | |
| A33 | 505.11 | |
| A34 | 496.14 | ¹H NMR (400 MHz, Methanol-d₄) δ 8.08 (s, 1H), |
| | | 8.03 (d, J = 8.5 Hz, 1H), 8.00-7.92 (m, 2H), 7.68- |
| | | 7.58 (m, 3H), 7.40 (d, J = 7.6 Hz, 1H), 7.31 (d, J = |
| | | 7.7 Hz, 1H), 5.13 (dd, J = 13.3, 5.1 Hz, 1H), 4.72 |
| | | (s, 2H), 4.56 (s, 2H), 4.50-4.36 (m, 2H), 3.68- |
| | | 3.57 (m, 2H), 3.29-3.18 (m, 2H), 2.98-2.83 (m, |
| | | 1H), 2.82-2.72 (m, 1H), 2.49 (qd, J = 13.2, 4.7 |
| | | Hz, 1H), 2.33-2.12 (m, 3H), 2.12-2.02 (m, 2H). |
| A35 | 514.10 | |
| A36 | 514.11 | |
| A37 | 530.07 | |
| A38 | 497.14 | |
| A39 | 497.15 | |
| A40 | 497.15 | |
| A41 | 497.17 | ¹H NMR (400 MHz, Methanol-d₄) δ 9.12-9.02 |
| | | (m, 1H), 8.48 (dd, J = 8.4, 1.7 Hz, 1H), 8.14 (dd, J = |
| | | 8.3, 1.3 Hz, 1H), 8.03-7.90 (m, 1H), 7.76-7.66 |
| | | (m, 2H), 7.49-7.28 (m, 2H), 5.13 (dd, J = 13.3, |
| | | 5.1 Hz, 1H), 4.99 (s, 2H), 4.74 (s, 2H), 4.51-4.36 |
| | | (m, 2H), 3.73-3.60 (m, 2H), 3.44-3.33 (m, 2H), |
| | | 2.96-2.84 (m, 1H), 2.83-2.72 (m, 1H), 2.57- |
| | | 2.43 (m, 1H), 2.38-2.24 (m, 2H), 2.21-2.12 (m, |
| | | 1H), 2.12-2.02 (m, 2H). |
| A42 | 497.14 | |
| A43 | 497.09 | |
| A44 | 497.08 | |
| A45 | 497.16 | |
| A46 | 497.16 | |
| A47 | 498.16 | |
| A48 | 498.16 | |
| A49 | 486.18 | ¹H NMR (400 MHz, Methanol-d₄) δ 8.40 (s, 1H), |
| | | 7.74 (d, J = 8.5 Hz, 1H), 7.58-7.51 (m, 1H), 7.43- |
| | | 7.36 (m, 2H), 7.29 (d, J = 7.7 Hz, 1H), 5.13 (dd, J = |
| | | 13.3, 5.1 Hz, 1H), 4.79-4.67 (m, 4H), 4.52- |
| | | 4.34 (m, 2H), 3.71-3.58 (m, 2H), 3.39-3.32 (m, |
| | | 2H), 2.97-2.83 (m, 1H), 2.83-2.73 (m, 1H), 2.58- |
| | | 2.43 (m, 1H), 2.30-2.13 (m, 3H), 2.13-2.04 |
| | | (m, 2H). |
| A50 | 514.09 | |
| A51 | 500.08 | |
| A52 | 486.14 | |
| A53 | 500.09 | |
| A54 | 502.98 | |
| A56 | 486.09 | |
| A57 | 500.05 | ¹H NMR (400 MHz, DMSO) δ 11.01 (s, 1H), 8.13 |
| | | (s, 1H), 7.88 (d, J = 7.7 Hz, 1H), 7.81 (s, 1 H), 7.46- |
| | | 7.17 (m, 3H), 5.19-5.03 (m, 1H), 4.68 (s, 2H), |
| | | 4.52 (s, 2H), 4.40 (d, J = 17.0 Hz, 1H), 4.24 (d, J = |
| | | 17.3 Hz, 1H), 4.09 (s, 3H), 3.44 (brs, 2H), 3.21 |
| | | (brs, 2H), 2.92 (t, J = 12.7 Hz, 1H), 2.60 (d, J = |
| | | 17.7 Hz, 1H), 2.43 (d, J = 12.5 Hz, 1H), 2.28-1.85 |
| | | (m, 5H) |
| A58 | 486.10 | |
| A59 | 485.10 | |
| A60 | 487.06 | |
| A62 | 503.02 | |
| A64 | 497.10 | |
| A65 | 497.08 | |
| A66 | 486.09 | |
| A67 | 500.10 | |
| A68 | 500.11 | |
| A69 | 500.08 | ¹H NMR (400 MHz, DMSO-d₆) δ 10.97 (s, 1H), |
| | | 8.42 (s, 1H), 7.83 (d, J = 8.5 Hz, 1H), 7.78 (s, 1H), |
| | | 7.33 (d, J = 7.6 Hz, 1H), 7.24 (d, J = 7.7 Hz, 1H), |
| | | 7.14 (dd, J = 8.6, 1.3 Hz, 1H), 5.08 (dd, J = 13.3, |
| | | 5.1 Hz, 1H), 4.67 (s, 2H), 4.50-4.35 (m, 3H), 4.29- |
| | | 4.16 (m, 4H), 3.23-3.10 (m, 2H), 2.99-2.84 |
| | | (m, 1H), 2.64-2.54 (m, 1H), 2.47-2.38 (m, 1H), |
| | | 2.20-2.05 (m, 2H), 2.03-1.91 (m, 3H). |
| A70 | 486.15 | |
| A71 | 486.14 | |
| A72 | 500.10 | |
| A73 | 486.12 | |
| A74 | 499.12 | |
| A75 | 500.12 | ¹H NMR (400 MHz, Methanol-d₄) δ 8.34 (s, 1H), |
| | | 7.87 (dd, J = 8.4, 0.8 Hz, 1H), 7.49 (d, J = 6.8 Hz, |
| | | 1H), 7.40 (d, J = 7.7 Hz, 1H), 7.31 (d, J = 7.7 Hz, |
| | | 1H), 7.19 (dd, J = 8.4, 6.9 Hz, 1H), 5.13 (dd, J = |
| | | 13.3, 5.1 Hz, 1H), 4.72 (s, 3H), 4.51-4.35 (m, |
| | | 2H), 4.30 (s, 3H), 3.73-3.63 (m, 2H), 3.37-3.33 |
| | | (m, 1H), 3.30-3.26 (m, 1H), 2.96-2.84 (m, 1H), |
| | | 2.83-2.73 (m, 1H), 2.57-2.43 (m, 1H), 2.32- |
| | | 2.12 (m, 3H), 2.12-2.02 (m, 2H). |
| A76 | 486.13 | |
| A77 | 486.09 | |
| A78 | 486.11 | |
| A79 | 486.29 | |
| A80 | 486.14 | |
| A81 | 529.23 | |
| A82 | 529.06 | |
| A83 | 513.13 | |
| A84 | 512.12 | |
| A85 | 529.09 | |
| A86 | 538.11 | |
| A87 | 485.11 | ¹H NMR (400 MHz, Methanol-d₄) δ 7.60-7.53 |
| | | (m, 1H), 7.43 (d, J = 3.2 Hz, 1H), 7.39 (d, J = 7.7 |
| | | Hz, 1H), 7.32-7.23 (m, 3H), 6.79-6.74 (m, 1H), |
| | | 5.13 (dd, J = 13.3, 5.1 Hz, 1H), 4.75-4.67 (m, |
| | | 2H), 4.64 (s, 2H), 4.53-4.35 (m, 2H), 3.69-3.57 |
| | | (m, 2H), 3.30-3.23 (m, 2H), 2.96-2.83 (m, 1H), |
| | | 2.82-2.73 (m, 1H), 2.56-2.42 (m, 1H), 2.28- |
| | | 2.11 (m, 3H), 2.10-2.00 (m, 2H). |
| A88 | 502.16 | |
| A89 | 528.14 | |
| A90 | 512.08 | |
| A91 | 525.92 | |
| A92 | 490.10 | |
| A93 | 478.21 | |
| A94 | 496.19 | |
| A95 | 503.08 | |
| A96 | 515.15 | |
| A97 | 531.09 | |
| A98 | 476.09 | |
| A99 | 490.07 | |
| A100 | 538.07 | |
| A101 | 539.07 | |
| A102 | 522.10 | |
| A103 | 523.09 | |
| A104 | 512.08 | ¹H NMR (400 MHz, Methanol-d₄) δ 8.04 (s, 2H), |
| | | 7.85-7.73 (m, 2H), 7.53 (t, J = 7.7 Hz, 1H), 7.48- |
| | | 7.36 (m, 2H), 7.31 (d, J = 7.7 Hz, 1H), 5.13 (dd, J = |
| | | 13.3, 5.1 Hz, 1H), 4.77-4.67 (m, 2H), 4.53-4.32 |
| | | (m, 4H), 3.68-3.50 (m, 2H), 3.28-3.15 (m, 2H), |
| | | 2.97-2.83 (m, 1H), 2.83-2.72 (m, 1H), 2.57- |
| | | 2.42 (m, 1H), 2.32-2.00 (m, 5H). |
| A105 | 526.12 | ¹H NMR (400 MHz, Methanol-d₄) δ 8.02 (s, 1H), |
| | | 7.88 (d, J = 0.7 Hz, 1H), 7.77-7.67 (m, 2H), 7.51 |
| | | (t, J = 7.7 Hz, 1H), 7.45-7.35 (m, 2H), 7.31 (d, J = |
| | | 7.7 Hz, 1H), 5.13 (dd, J = 13.3, 5.1 Hz, 1H), 4.75- |
| | | 4.66 (m, 2H), 4.51-4.33 (m, 4H), 3.61 (d, J = |
| | | 13.0 Hz, 2H), 3.28-3.15 (m, 2H), 2.97-2.83 (m, |
| | | 1H), 2.82-2.72 (m, 1H), 2.56-2.43 (m, 1H), 2.35- |
| | | 2.02 (m, 5H). |
| A106 | 529.04 | |
| A107 | 512.09 | |
| A108 | 512.07 | |
| A109 | 498.07 | |
| A110 | 503.05 | ¹H NMR (400 MHz, Methanol-d₄) δ 7.48 (d, J = |
| | | 3.2 Hz, 1H), 7.46-7.37 (m, 2H), 7.35-7.25 (m, |
| | | 1H), 7.21-7.09 (m, 1H), 6.60 (d, J = 3.2 Hz, 1H), |
| | | 5.13 (dd, J = 13.3, 5.1 Hz, 1H), 4.73 (s, 2H), 4.63 |
| | | (s, 2H), 4.50-4.35 (m, 2H), 3.71-3.60 (m, 2H), |
| | | 3.30-3.19 (m, 2H), 2.96-2.84 (m, 1H), 2.83- |
| | | 2.73 (m, 1H), 2.57-2.42 (m, 1H), 2.28-2.05 (m, |
| | | 5H). |
| A111 | 513.09 | |
| A112 | 490.20 | |
| A113 | 488.17 | ¹H NMR (400 MHz, Methanol-d₄) δ 7.50-7.39 |
| | | (m, 4H), 7.36-7.28 (m, 1H), 5.31-5.06 (m, 5H), |
| | | 4.72 (s, 2H), 4.55-4.37 (m, 2H), 4.34 (s, 2H), 3.67- |
| | | 3.55 (m, 2H), 3.26-3.18 (m, 2H), 2.93-2.86 |
| | | (m, 1H), 2.82-2.74 (m, 1H), 2.57-2.43 (m, 1H), |
| | | 2.32-2.04 (m, 5H). |
| A114 | 553.16 | |
| A115 | 486.18 | |
| A116 | 486.22 | |
| A117 | 504.06 | |
| A118 | 504.07 | |
| A119 | 486.11 | ¹H NMR (400 MHz, Methanol-d₄) δ 7.96 (d, J = |
| | | 2.3 Hz, 1H), 7.74-7.66 (m, 1H), 7.54-7.46 (m, |
| | | 2H), 7.41 (d, J = 7.7 Hz, 1H), 7.29 (d, J = 7.6 Hz, |
| | | 1H), 7.24-7.18 (m, 1H), 5.13 (dd, J = 13.3, 5.1 |
| | | Hz, 1H), 4.73 (s, 2H), 4.65 (s, 2H), 4.51-4.35 (m, |
| | | 2H), 3.69-3.55 (m, 2H), 3.30-3.27 (m, 2H), 2.99- |
| | | 2.84 (m, 1H), 2.83-2.72 (m, 1H), 2.58-2.43 |
| | | (m, 1H), 2.29-2.04 (m, 5H). |
| A120 | 486.09 | ¹H NMR (400 MHz, Methanol-d₄) δ 7.88 (d, J = |
| | | 2.2 Hz, 1H), 7.86-7.81 (m, 1H), 7.67 (d, J = 8.5 |
| | | Hz, 1H), 7.51-7.44 (m, 1H), 7.41 (d, J = 7.7 Hz, |
| | | 1H), 7.31 (d, J = 7.6 Hz, 1H), 6.95 (dd, J = 2.2, 1.0 |
| | | Hz, 1H), 5.13 (dd, J = 13.3, 5.1 Hz, 1H), 4.71 (s, |
| | | 2H), 4.53-4.36 (m, 4H), 3.65-3.49 (m, 2H), 3.26- |
| | | 3.15 (m, 2H), 2.96-2.84 (m, 1H), 2.82-2.73 |
| | | (m, 1H), 2.58-2.43 (m, 1H), 2.28-2.13 (m, 3H), |
| | | 2.13-2.03 (m, 2H). |
| A121 | 499.13 | ¹H NMR (400 MHz, Methanol-d₄) δ 7.46-7.37 |
| | | (m, 2H), 7.28 (d, J = 7.7 Hz, 1H), 7.21-7.12 (m, |
| | | 2H), 5.13 (dd, J = 13.3, 5.1 Hz, 1H), 4.75-4.67 |
| | | (m, 2H), 4.57 (s, 2H), 4.53-4.36 (m, 3H), 3.66- |
| | | 3.57 (m, 2H), 3.30-3.21 (m, 2H), 2.96-2.84 (m, |
| | | 1H), 2.82-2.73 (m, 1H), 2.56-2.43 (m, 4H), 2.28- |
| | | 2.12 (m, 3H), 2.11-2.01 (m, 2H). |
| A122 | 501.04 | |
| A123 | 529.11 | |
| A124 | 513.10 | |
| A125 | 513.10 | |
| A126 | 526.11 | |
| A127 | 563.92 | |
| A128 | 520.02 | |
| A129 | 485.10 | |
| A130 | 499.12 | |
| A131 | 501.02 | |
| A132 | 523.12 | |
| A133 | 523.11 | |
| A134 | 526.08 | |
| A135 | 553.16 | |
| A136 | 504.03 | |
| A137 | 503.07 | |
| A138 | 515.11 | |
| A139 | 526.06 | |
| A140 | 518.02 | |
| A141 | 519.04 | |
| A142 | 520.02 | |
| A143 | 500.12 | |
| A144 | 526.10 | |
| A145 | 540.09 | ¹H NMR (400 MHz, Methanol-d₄) δ 8.10-8.06 |
| | | (m, 1H), 7.89 (d, J = 0.7 Hz, 1H), 7.76 (s, 1H), 7.73- |
| | | 7.68 (m, 1H), 7.50 (t, J = 7.7 Hz, 1H), 7.45-7.35 |
| | | (m, 2H), 7.31 (d, J = 7.7 Hz, 1H), 5.13 (dd, J = |
| | | 13.3, 5.2 Hz, 1H), 4.71 (s, 2H), 4.51-4.34 (m, |
| | | 4H), 4.23 (q, J = 7.3 Hz, 2H), 3.67-3.55 (m, 2H), |
| | | 3.27-3.15 (m, 2H), 2.95-2.82 (m, 1H), 2.82- |
| | | 2.73 (m, 1H), 2.35-2.20 (m, 2H), 2.20-2.11 (m, |
| | | 1H), 2.11-2.01 (m, 2H), 1.50 (t, J = 7.3 Hz, 3H). |
| A146 | 554.13 | ¹H NMR (400 MHz, Methanol-d₄) δ 8.11 (d, J = |
| | | 0.8 Hz, 1H), 7.90 (d, J = 0.8 Hz, 1H), 7.78-7.69 |
| | | (m, 2H), 7.52 (t, J = 7.7 Hz, 1H), 7.44-7.34 (m, |
| | | 2H), 7.31 (d, J = 7.7 Hz, 1H), 5.13 (dd, J = 13.3, |
| | | 5.1 Hz, 1H), 4.72 (s, 2H), 4.62-4.54 (m, 1H), 4.51- |
| | | 4.36 (m, 4H), 3.67-3.56 (m, 2H), 3.27-3.16 |
| | | (m, 2H), 2.97-2.84 (m, 1H), 2.83-2.72 (m, 1H), |
| | | 2.57-2.43 (m, 1H), 2.30-2.05 (m, 5H), 1.54 (d, J = |
| | | 6.7 Hz, 6H). |
| A147 | 596.10 | ¹H NMR (400 MHz, Methanol-d₄) δ 8.18-8.12 |
| | | (m, 1H), 7.92 (d, J = 0.6 Hz, 1H), 7.78-7.70 (m, |
| | | 2H), 7.52 (t, J = 7.7 Hz, 1H), 7.47-7.36 (m, 2H), |
| | | 7.31 (d, J = 7.6 Hz, 1H), 5.13 (dd, J = 13.3, 5.1 Hz, |
| | | 1H), 4.72 (s, 2H), 4.51-4.34 (m, 5H), 4.14-4.03 |
| | | (m, 2H), 3.68-3.54 (m, 4H), 3.28-3.15 (m, 2H), |
| | | 2.98-2.83 (m, 1H), 2.82-2.72 (m, 1H), 2.57- |
| | | 2.43 (m, 1H), 2.30-2.02 (m, 9H). |
| A148 | 526.11 | |
| A149 | 512.09 | |
| A150 | 514.08 | |
| A151 | 528.09 | |
| A152 | 501.10 | |
| A153 | 517.10 | |
| A154 | 576.07 | |
| A155 | 526.11 | |
| A156 | 512.12 | |
| A157 | 553.07 | |
| A158 | 529.04 | |
| A159 | 539.05 | |
| A160 | 512.10 | ¹H NMR (400 MHz, Methanol-d₄) δ 7.98 (s, 1H), |
| | | 7.96-7.91 (m, 1H), 7.74 (d, J = 2.3 Hz, 1H), 7.58 |
| | | (t, J = 7.7 Hz, 1H), 7.50 (d, J = 7.6 Hz, 1H), 7.41 |
| | | (d, J = 7.6 Hz, 1H), 7.31 (d, J = 7.6 Hz, 1H), 6.76 |
| | | (d, J = 2.3 Hz, 1H), 5.13 (dd, J = 13.3, 5.2 Hz, 1H), |
| | | 4.71 (s, 2H), 4.51-4.35 (m, 4H), 3.68-3.51 (m, |
| | | 2H), 3.29-3.16 (m, 2H), 2.96-2.83 (m, 1H), 2.82- |
| | | 2.72 (m, 1H), 2.58-2.43 (m, 1H), 2.32-2.12 |
| | | (m, 3H), 2.12-2.03 (m, 2H). |
| A161 | 568.09 | |
| A162 | 512.11 | |
| A163 | 486.05 | ¹H NMR (400 MHz, Methanol-d₄) δ 8.36 (s, 1H), |
| | | 8.22 (s, 1H), 7.53 (d, J = 3.5 Hz, 1H), 7.41 (d, J = |
| | | 7.6 Hz, 1H), 7.30 (d, J = 7.7 Hz, 1H), 6.61 (d, J = |
| | | 3.5 Hz, 1H), 5.13 (dd, J = 13.3, 5.2 Hz, 1H), 4.71 |
| | | (s, 2H), 4.59-4.34 (m, 4H), 3.70-3.50 (m, 2H), |
| | | 3.29-3.16 (m, 2H), 2.97-2.84 (m, 1H), 2.83- |
| | | 2.72 (m, 1H), 2.57-2.42 (m, 1H), 2.29-2.04 (m, |
| | | 5H). |
| A164 | 528.06 | |
| A165 | 503.04 | |
| A166 | 597.05 | |
| A167 | 556.07 | |
| A168 | 553.07 | ¹H NMR (400 MHz, Methanol-d₄) δ 8.19-8.09 |
| | | (m, 1H), 7.76-7.69 (m, 1H), 7.66 (s, 1H), 7.62 (d, |
| | | J = 7.4 Hz, 1H), 7.57-7.46 (m, 2H), 7.41 (d, J = |
| | | 7.7 Hz, 1H), 7.31 (d, J = 7.7 Hz, 1H), 7.04-6.94 |
| | | (m, 1H), 6.89 (d, J = 8.4 Hz, 1H), 5.44 (s, 2H), 5.13 |
| | | (dd, J = 13.3, 5.2 Hz, 1H), 4.70 (s, 2H), 4.55-4.34 |
| | | (m, 4H), 3.63-3.50 (m, 2H), 3.19 (t, J = 13.2 Hz, |
| | | 2H), 2.97-2.84 (m, 1H), 2.83-2.72 (m, 1H), 2.58- |
| | | 2.42 (m, 1H), 2.30-2.13 (m, 3H), 2.12-2.02 |
| | | (m, 2H). |
| A169 | 500.08 | |
| A170 | 526.17 | ¹H NMR (400 MHz, DMSO-d₆) δ 10.96 (s, 1H), |
| | | 8.13 (s, 1H), 7.84 (d, J = 0.7 Hz, 1H), 7.49 (s, 1H), |
| | | 7.47-7.38 (m, 2H), 7.35-7.23 (m, 2H), 7.16 (d, J = |
| | | 7.6 Hz, 1H), 5.08 (dd, J = 13.3, 5.1 Hz, 1H), 4.53 |
| | | (s, 2H), 4.37 (d, J = 17.1 Hz, 1H), 4.21 (d, J = 17.1 |
| | | Hz, 1H), 3.87 (s, 3H), 3.51 (s, 2H), 2.98-2.76 (m, |
| | | 3H), 2.63-2.54 (m, 1H), 2.48-2.35 (m, 1H), 2.13- |
| | | 2.00 (m, 2H), 2.00-1.86 (m, 3H), 1.76-1.60 |
| | | (m, 2H). |
| A171 | 501.12 | ¹H NMR (400 MHz, Methanol-d₄) δ 7.96 (s, 1H), |
| | | 7.81 (d, J = 7.9 Hz, 1H), 7.75 (d, J = 7.8 Hz, 1H), |
| | | 7.41 (d, J = 7.7 Hz, 1H), 7.31 (d, J = 7.8 Hz, 1H), |
| | | 5.14 (dd, J = 13.3, 5.2 Hz, 1H), 4.71 (s, 2H), 4.58- |
| | | 4.35 (m, 6H), 3.64-3.50 (m, 2H), 3.29-3.16 (m, |
| | | 2H), 2.97-2.84 (m, 1H), 2.83-2.73 (m, 1H), 2.57- |
| | | 2.43 (m, 1H), 2.30-2.12 (m, 3H), 2.12-2.02 |
| | | (m, 2H). |
| A172 | 485.14 | |
| A173 | 500.94 | ¹H NMR (400 MHz, Methanol-d₄) δ 7.45-7.36 |
| | | (m, 2H), 7.31 (d, J = 7.6 Hz, 1H), 7.16 (d, J = 7.7 |
| | | Hz, 1H), 7.07 (s, 1H), 5.14 (dd, J = 13.3, 5.1 Hz, |
| | | 1H), 4.70 (s, 2H), 4.51-4.33 (m, 4H), 3.66-3.53 |
| | | (m, 4H), 3.23-3.12 (m, 2H), 2.98-2.84 (m, 1H), |
| | | 2.83-2.73 (m, 1H), 2.57-2.43 (m, 1H), 2.32- |
| | | 2.03 (m, 5H). |
| A174 | 552.10 | ¹H NMR (400 MHz, Methanol-d₄) δ 8.12 (t, J = 0.6 |
| | | Hz, 1H), 7.88 (d, J = 0.9 Hz, 1H), 7.76-7.69 (m, |
| | | 2H), 7.51 (t, J = 7.7 Hz, 1H), 7.45-7.35 (m, 2H), |
| | | 7.31 (d, J = 7.6 Hz, 1H), 5.13 (dd, J = 13.3, 5.1 Hz, |
| | | 1H), 4.75-4.68 (m, 2H), 4.52-4.33 (m, 4H), 3.71 |
| | | (tt, J = 7.3, 3.9 Hz, 1H), 3.65-3.56 (m, 2H), 3.26- |
| | | 3.16 (m, 2H), 2.97-2.84 (m, 1H), 2.84-2.72 (m, |
| | | 1H), 2.58-2.42 (m, 1H), 2.31-2.05 (m, 5H). |
| A175 | 517.05 | |
| A176 | 503.06 | |
| A177 | 595.11 | ¹H NMR (400 MHz, Methanol-d₄) δ 8.11 (s, 1H), |
| | | 8.01-7.90 (m, 2H), 7.61 (d, J = 8.2 Hz, 1H), 7.52 |
| | | (t, J = 7.9 Hz, 1H), 7.42 (d, J = 7.7 Hz, 1H), 7.32 |
| | | (d, J = 7.7 Hz, 2H), 7.12-6.98 (m, 2H), 5.14 (dd, J = |
| | | 13.3, 5.2 Hz, 1H), 4.72 (s, 2H), 4.55-4.31 (m, |
| | | 4H), 3.89 (s, 3H), 3.69-3.56 (m, 2H), 3.27-3.12 |
| | | (m, 2H), 2.97-2.84 (m, 1H), 2.83-2.72 (m, 1H), |
| | | 2.58-2.42 (m, 1H), 2.33-2.03 (m, 5H). |
| A178 | 598.99 | ¹H NMR (400 MHz, Methanol-d₄) δ 8.13 (s, 1H), |
| | | 7.99-7.91 (m, 2H), 7.68-7.60 (m, 1H), 7.58- |
| | | 7.50 (m, 3H), 7.42 (d, J = 7.7 Hz, 1H), 7.37-7.27 |
| | | (m, 2H), 5.14 (dd, J = 13.3, 5.1 Hz, 1H), 4.77- |
| | | 4.66 (m, 2H), 4.51-4.35 (m, 4H), 3.68-3.57 (m, |
| | | 2H), 3.29-3.16 (m, 2H), 2.98-2.84 (m, 1H), 2.83- |
| | | 2.72 (m, 1H), 2.59-2.42 (m, 1H), 2.33-2.04 |
| | | (m, 5H). |
| A179 | 579.09 | |
| A180 | 486.13 | |
| A181 | 528.13 | |
| A182 | 452.03 | |
| A183 | 452.03 | |
| A184 | 502.05 | |
| A185 | 502.03 | ¹H NMR (400 MHz, Methanol-d₄) δ 8.08 (d, J = |
| | | 8.1 Hz, 1H), 8.05-7.99 (m, 2H), 7.62-7.53 (m, |
| | | 1H), 7.53-7.46 (m, 1H), 7.41 (d, J = 7.7 Hz, 1H), |
| | | 7.29 (d, J = 7.5 Hz, 1H), 5.13 (dd, J = 13.3, 5.2 Hz, |
| | | 1H), 4.77-4.63 (m, 4H), 4.54-4.34 (m, 2H), 3.71- |
| | | 3.58 (m, 2H), 3.28-3.17 (m, 2H), 2.98-2.83 |
| | | (m, 1H), 2.82-2.71 (m, 1H), 2.56-2.41 (m, 1H), |
| | | 2.28-2.05 (m, 5H). |
| A186 | 502.04 | ¹H NMR (400 MHz, Methanol-d₄) δ 8.11 (d, J = |
| | | 8.1 Hz, 1H), 7.84 (d, J = 5.6 Hz, 1H), 7.78 (d, J = |
| | | 5.7 Hz, 1H), 7.62 (d, J = 7.2 Hz, 1H), 7.55-7.47 |
| | | (m, 1H), 7.40 (d, J = 7.6 Hz, 1H), 7.28 (d, J = 7.6 |
| | | Hz, 1H), 5.13 (dd, J = 13.3, 5.2 Hz, 1H), 4.80- |
| | | 4.68 (m, 4H), 4.53-4.34 (m, 2H), 3.68-3.57 (m, |
| | | 2H), 3.39-3.32 (m, 2H), 2.97-2.83 (m, 1H), 2.82- |
| | | 2.73 (m, 1H), 2.58-2.41 (m, 1H), 2.30-2.12 |
| | | (m, 3H), 2.11-2.01 (m, 2H). |
| A187 | 502.05 | |
| A188 | 502.06 | |
| A189 | 502.04 | |
| A190 | 531.97 | |
| A191 | 488.26 | |
| A192 | 464.20 | |
| A193 | 526.51 | |
| A194 | 514.22 | |
| A195 | 471.19 | |
| A196 | 485.20 | |
| A197 | 512.40 | |
| A198 | 486.24 | |
| A199 | 504.24 | |
| A200 | 504.26 | |
| A201 | 517.24 | |
| A202 | 491.22 | |
| A203 | 488.28 | |
| A204 | 486.24 | |
| A205 | 486.24 | |
| A206 | 448.20 | |
| A207 | 486.24 | ¹H NMR (400 MHz, Methanol-d₄) δ 7.51-7.23 |
| | | (m, 5H), 5.15 (dd, J = 13.3, 5.2 Hz, 1H), 4.74 (s, |
| | | 2H), 4.60-4.27 (m, 4H), 3.62 (t, J = 10.0 Hz, 2H), |
| | | 3.26 (t, J = 13.0 Hz, 2H), 3.09 (t, J = 7.4 Hz, 2H), |
| | | 3.02 (t, J = 7.4 Hz, 2H), 2.92 (ddd, J = 17.6, 13.5, |
| | | 5.4 Hz, 1H), 2.80 (ddd, J = 17.6, 4.7, 2.4 Hz, 1H), |
| | | 2.59-2.44 (m, 1H), 2.38-2.04 (m, 7H). |
| A208 | 500.26 | ¹H NMR (400 MHz, Methanol-d₄) δ 7.43 (d, J = |
| | | 7.7 Hz, 1H), 7.32 (t, J = 6.0 Hz, 2H), 7.28-7.19 |
| | | (m, 2H), 5.15 (dd, J = 13.3, 5.1 Hz, 1H), 4.80- |
| | | 4.70 (m, 2H), 4.54-4.34 (m, 4H), 3.65 (d, J = 12.9 |
| | | Hz, 2H), 3.28 (d, J = 12.8 Hz, 2H), 3.01-2.73 (m, |
| | | 6H), 2.60-2.43 (m, 1H), 2.28 (t, J = 14.4 Hz, 2H), |
| | | 2.19 (ddq, J = 10.5, 5.3, 2.7 Hz, 1H), 2.09 (d, J = |
| | | 14.9 Hz, 2H), 1.94 (p, J = 6.4 Hz, 2H), 1.90-1.77 |
| | | (m, 2H). |
| A209 | 526.26 | ¹H NMR (400 MHz, Methanol-d₄) δ 7.95 (s, 1H), |
| | | 7.72 (s, 1H), 7.46-7.26 (m, 7H), 5.40 (s, 2H), 5.15 |
| | | (dd, J = 13.3, 5.2 Hz, 1H), 4.69 (s, 2H), 4.57-4.37 |
| | | (m, 3H), 4.31 (s, 2H), 3.62 (d, J = 12.9 Hz, 2H), |
| | | 3.10 (d, J = 13.1 Hz, 2H), 2.97-2.86 (m, 1H), 2.80 |
| | | (ddd, J = 17.6, 4.7, 2.4 Hz, 1H), 2.52 (qd, J = 13.2, |
| | | 4.8 Hz, 1H), 2.26-2.06 (m, 5H). |
| A210 | 553.21 | |
| A211 | 476.20 | |
| A212 | 476.20 | |
| A213 | 460.25 | |
| A214 | 480.20 | |
| A215 | 476.20 | |
| A216 | 480.20 | |
| A217 | 500.26 | ¹H NMR (400 MHz, Methanol-d₄) δ 8.12 (d, J = |
| | | 0.9 Hz, 1H), 8.03-7.95 (m, 1H), 7.76-7.70 (m, |
| | | 1H), 7.59 (d, J = 8.8 Hz, 1H), 7.42 (d, J = 7.6 Hz, |
| | | 1H), 7.32 (d, J = 7.7 Hz, 1H), 5.15 (dd, J = 13.4, |
| | | 5.1 Hz, 1H), 4.76-4.65 (m, 2H), 4.52-4.37 (m, |
| | | 3H), 4.13 (s, 3H), 3.61 (d, J = 12.4 Hz, 2H), 3.22 (t, |
| | | J = 13.1 Hz, 2H), 2.92 (ddd, J = 18.5, 13.4, 5.4 Hz, |
| | | 1H), 2.79 (ddd, J = 17.8, 4.8, 2.5 Hz, 1H), 2.51 (qd, |
| | | J = 13.2, 4.7 Hz, 1H), 2.30-2.13 (m, 2H), 2.08 (d, |
| | | J = 14.9 Hz, 2H). |
| A218 | 502.24 | |
| A219 | 471.20 | |
| A220 | 518.20 | |
| A221 | 512.24 | |
| A222 | 512.20 | |
| A223 | 504.20 | |
| A224 | 488.26 | |
| A225 | 490.23 | |
| A226 | 500.20 | |
| A227 | 486.24 | |
| A228 | 450.51 | |
| A229 | 464.22 | |
| A230 | 476.22 | |
| A231 | 490.24 | |
| A232 | 518.19 | |
| A233 | 534.24 | |
| A234 | 524.20 | |
| A235 | 524.20 | |
| A236 | 524.20 | |
| A237 | 544.20 | |
| A238 | 544.22 | |
| A239 | 544.20 | |
| A240 | 560.20 | |
| A241 | 560.20 | |
| A242 | 560.20 | |
| A243 | 546.10 | |
| A244 | 476.22 | |
| A245 | 490.20 | |
| A246 | 512.24 | |
| A247 | 491.22 | |
| A248 | 481.16 | |
| A249 | 481.16 | |
| A250 | 481.16 | |
| A251 | 512.21 | |
| A252 | 491.22 | |
| A253 | 513.22 | |
| A254 | 513.22 | |
| A255 | 520.25 | |
| A256 | 526.24 | |
| A257 | 484.17 | |
| A258 | 356.03 | ¹H NMR (400 MHz, Methanol-d₄) δ 7.81 (d, J = |
| | | 7.8 Hz, 1H), 7.43 (d, J = 7.8 Hz, 1H), 5.21-5.13 |
| | | (m, 3H), 4.56-4.39 (m, 2H), 3.48-3.36 (m, 4H), |
| | | 2.97-2.86 (m, 1H), 2.83-2.75 (m, 1H), 2.59- |
| | | 2.44 (m, 1H), 2.34-2.15 (m, 3H), 2.02-1.94 (m, |
| | | 2H). |
| A259 | 526.16 | |
| A260 | 512.17 | |
| A261 | 600.83 | |
| A262 | 595.05 | |
| A263 | 579.09 | |
| A264 | 485.14 | |
| A265 | 568.11 | |
| A266 | 486.14 | |
| A267 | 526.14 | |
| A268 | 552.12 | |
| A269 | 582.15 | |
| A270 | 582.26 | |
| A271 | 582.17 | |
| A272 | 612.17 | |
| A273 | 582.16 | |
| A274 | 582.17 | |
| A275 | 472.21 | |
| A276 | 586.16 | |
| A277 | 598.18 | |
| A278 | 557.04 | |
| A279 | 582.14 | |
| A280 | 556.10 | |
| A281 | 562.08 | |
| A282 | 580.07 | |
| A283 | 540.15 | |
| A284 | 540.16 | |
| A285 | 542.10 | |
| A286 | 570.41 | |
| A287 | 544.28 | |
| A288 | 536.34 | |
| A289 | 562.34 | |
| A290 | 512.11 | |
| A291 | 529.39 | |
| A292 | 584.45 | |
| A293 | 558.36 | |
| A294 | 556.40 | |
| A295 | 514.33 | |
| A296 | 527.36 | |
| A297 | 596.45 | |
| A298 | 514.32 | |
| A299 | 514.31 | |
| A300 | 503.35 | |
| A301 | 503.32 | |
| A302 | 529.36 | |
| A303 | 515.29 | |
| A304 | 516.32 | |
| A305 | 557.34 | |
| A306 | 369.32 | ¹H NMR (400 MHz, Methanol-d₄) δ 7.21 (s, 2H), |
| | | 5.14 (dd, J = 13.3, 5.2 Hz, 1H), 4.72-4.55 (m, |
| | | 2H), 3.51-3.39 (m, 4H), 3.24-3.13 (m, 2H), 2.99 |
| | | (s, 3H), 2.96-2.83 (m, 1H), 2.83-2.74 (m, 1H), |
| | | 2.60-2.46 (m, 1H), 2.22-2.03 (m, 3H), 2.03- |
| | | 1.94 (m, 2H). |
| A307 | 355.27 | ¹H NMR (400 MHz, Methanol-d₄) δ 7.28-7.17 |
| | | (m, 2H), 5.14 (dd, J = 13.3, 5.2 Hz, 1H), 4.39- |
| | | 4.23 (m, 2H), 3.67 (s, 2H), 3.51-3.39 (m, 2H), |
| | | 3.25-3.12 (m, 2H), 2.98-2.84 (m, 1H), 2.84- |
| | | 2.72 (m, 1H), 2.56-2.39 (m, 1H), 2.23-1.96 (m, |
| | | 5H). |
| A308 | 539.20 | |
| A309 | 525.19 | |
| A310 | 530.16 | |
| A311 | 530.11 | |
| A312 | 530.14 | |
| A313 | 546.20 | |
| A314 | 546.23 | ¹H NMR (400 MHz, Methanol-d₄) δ 8.51 (s, 1H), |
| | | 7.93 (s, 1H), 7.89-7.75 (m, 2H), 7.56 (d, J = 8.7 |
| | | Hz, 2H), 7.44 (d, J = 7.7 Hz, 1H), 7.36 (d, J = 7.7 |
| | | Hz, 1H), 5.15 (dd, J = 13.3, 5.1 Hz, 1H), 4.74 (d, J = |
| | | 11.9 Hz, 2H), 4.53-4.33 (m, 4H), 3.69 (d, J = |
| | | 12.9 Hz, 2H), 3.27-3.11 (m, 2H), 3.01-2.74 (m, |
| | | 2H), 2.52 (qd, J = 13.2, 4.8 Hz, 1H), 2.39-2.05 |
| | | (m, 5H). |
| A315 | 546.18 | ¹H NMR (400 MHz, Methanol-d₄) δ 8.40 (d, J = |
| | | 2.5 Hz, 1H), 7.96-7.79 (m, 2H), 7.62-7.50 (m, |
| | | 2H), 7.50-7.33 (m, 2H), 6.77 (d, J = 2.5 Hz, 1H), |
| | | 5.15 (dd, J = 13.3, 5.1 Hz, 1H), 4.73 (s, 2H), 4.59- |
| | | 4.35 (m, 4H), 3.75 (d, J = 12.9 Hz, 2H), 3.27-3.20 |
| | | (m, 2H), 2.98-2.73 (m, 2H), 2.61-2.43 (m, 1H), |
| | | 2.40-2.05 (m, 5H). |
| A316 | 530.24 | |
| A317 | 446.12 | |
| A318 | 460.24 | |
| A319 | 506.09 | |
| A320 | 520.28 | |
| A321 | 546.12 | |
| A322 | 530.22 | |
| A323 | 530.14 | |
| A324 | 501.23 | |
| A325 | 501.18 | |
| A326 | 516.09 | |
| A327 | 530.20 | |
| A328 | 586.18 | |
| A329 | 582.28 | |
| A330 | 474.20 | |
| A331 | 546.12 | |
| A332 | 486.10 | |
| A333 | 486.13 | |
| A334 | | |
| A335 | 500.24 | |
| A336 | 576.12 | |
| A337 | 594.10 | ¹H NMR (400 MHz, Methanol-d₄) δ 7.97-7.78 |
| | | (m, 3H), 7.54-7.45 (m, 1H), 7.42 (d, J = 7.7 Hz, |
| | | 1H), 7.34 (d, J = 7.5 Hz, 1H), 7.29-7.13 (m, 2H), |
| | | 5.15 (dd, J = 13.3, 5.1 Hz, 1H), 4.70 (s, 2H), 4.62- |
| | | 4.32 (m, 4H), 3.62 (d, J = 12.7 Hz, 2H), 3.24-3.06 |
| | | (m, 2H), 3.00-2.75 (m, 2H), 2.57-2.46 (m, 1H), |
| | | 2.36-1.99 (m, 5H). |
| A338 | 553.12 | |
| A339 | 553.14 | |
| A340 | 585.98 | |
| A341 | 544.12 | |
| A342 | 544.20 | |
| A343 | 544.22 | |
| A344 | 530.24 | |
| A345 | 530.20 | |
| A346 | 554.22 | |
| A347 | 566.14 | |
| A348 | 566.18 | |
| A349 | 610.18 | |
| A350 | 610.11 | |
| A351 | 534.12 | ¹H NMR (400 MHz, Methanol-d₄) δ 7.46 (d, J = |
| | | 7.7 Hz, 1H), 7.38 (d, J = 7.8 Hz, 1H), 7.02 (ddt, J = |
| | | 8.4, 6.7, 2.5 Hz, 2H), 6.84 (ddd, J = 9.0, 6.7, 4.5 |
| | | Hz, 2H), 4.70-4.60 (m, 2H), 4.55-4.37 (m, 3H), |
| | | 3.63 (d, J = 12.9 Hz, 2H), 3.24-3.06 (m, 2H), 3.15 |
| | | (d, J = 25.8 Hz, 2H), 3.00-2.77 (m, 4H), 2.59- |
| | | 2.40 (m, 3H), 2.35-1.94 (m, 7H). |
| A352 | 610.01 | |
| A353 | 612.52 | |
| A354 | 594.18 | |
| A355 | 594.21 | |
| A356 | 534.26 | |
| A357 | 628.10 | |
| A358 | 534.08 | |
| A359 | 537.12 | ¹H NMR (400 MHz, Methanol-d₄) δ 8.66 (s, 1H), |
| | | 8.10-8.01 (m, 2H), 7.98 (d, J = 1.9 Hz, 1H), 7.95- |
| | | 7.88 (m, 2H), 7.42 (d, J = 7.7 Hz, 1H), 7.33 (d, J = |
| | | 7.7 Hz, 1H), 5.14 (dd, J = 13.3, 5.1 Hz, 1H), 4.68 |
| | | (s, 2H), 4.53-4.31 (m, 4H), 3.67 (t, J = 13.1 Hz, |
| | | 2H), 3.17 (t, J = 12.9 Hz, 2H), 2.99-2.73 (m, 2H), |
| | | 2.51 (qd, J = 13.2, 4.8 Hz, 1H), 2.40-1.88 (m, |
| | | 5H). |
| A360 | 580.18 | ¹H NMR (400 MHz, Methanol-d₄) δ 8.64 (s, 1H), |
| | | 8.06 (d, J = 8.5 Hz, 2H), 7.98 (s, 1H), 7.87 (d, J = |
| | | 8.6 Hz, 2H), 7.44 (d, J = 7.7 Hz, 1H), 7.36 (d, J = |
| | | 7.7 Hz, 1H), 5.15 (dd, J = 13.3, 5.2 Hz, 1H), 4.73 |
| | | (s, 2H), 4.57-4.34 (m, 4H), 3.70 (d, J = 12.9 Hz, |
| | | 2H), 3.27-3.12 (m, 2H), 3.01-2.74 (m, 2H), 2.52 |
| | | (qd, J = 13.2, 4.8 Hz, 1H), 2.38-2.07 (m, 5H). |
| A361 | 522.16 | |
| A362 | 504.20 | ¹H NMR (400 MHz, Methanol-d₄) δ 7.44 (d, J = |
| | | 7.7 Hz, 1H), 7.37 (d, J = 7.6 Hz, 1H), 7.19 (dd, J = |
| | | 8.7, 5.3 Hz, 2H), 7.03 (t, J = 8.8 Hz, 2H), 5.15 (dd, |
| | | J = 13.3, 5.2 Hz, 1H), 4.71 (s, 2H), 4.58-4.34 (m, |
| | | 2H), 3.75 (d, J = 12.8 Hz, 2H), 3.31-3.08 (m, 4H), |
| | | 2.98-2.86 (m, 1H), 2.80 (ddd, J = 17.6, 4.7, 2.4 |
| | | Hz, 1H), 2.52 (dd, J = 13.1, 4.8 Hz, 1H), 2.30 (d, J = |
| | | 14.6 Hz, 2H), 2.19 (dtd, J = 12.9, 5.3, 2.4 Hz, |
| | | 1H), 2.15-2.00 (m, 3H), 1.47 (dt, J = 8.0, 5.2 Hz, |
| | | 1H), 1.20 (ddt, J = 29.8, 9.0, 5.4 Hz, 2H). |

### For Compound B-1 to B-58

### Intermediate B1: tert-butyl 4-(chloromethyl)-3,6-dihydro-2H-pyridine-1-carboxylate

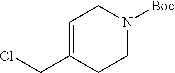 [SMILES: CC(C)(C)OC(=O)N1CC=C(CCl)CC1]

### Step A: tert-butyl 3-hydroxy-4-methylenepiperidine-1-carboxylate

The suspension of SeO₂ (61.8 g, 558 mmol, 0.55 eq) in DCM (3000 mL) was cooled to −10° C., before 2-hydroperoxy-2-methyl-propane in H₂O (274 g, 291 mL, 2.10 eq, 70% purity) was added dropwise, and the resulting mixture was stirred for 30 min at −10° C. The reaction mixture was further cooled to −30° C., before a solution of compound 5-1 (200 g, 1.01 mol, 1 eq) in DCM (1000 mL) was added dropwise, and the resulting mixture was stirred for another 1 hr at −30° C. The reaction mixture was warmed to 20° C., and stirred for further 18 hrs, before the mixture was cooled to 0° C., and was added ice chips and water (1.0 L). The resulting mixture was stirred at 0° C. for 30 min. The organic phase was separated, and the aqueous phase was extracted with DCM (500 mL), before the combined organic phase was added 10% w/v NaHSO₃ solution (1000 mL) portion-wise at 0° C., during which period the temperature was maintained below 10° C., and the mixture was stirred for further 5 min after the addition. The organic phase was separated, and the aqueous phase was extracted with DCM (500 mL). The combined organic phase was washed with brine (1000 mL), dried over anhydrous Na₂SO₄, filtered, and concentrated in vacuo. The crude product was purified by silica gel column chromatography (Petroleum ether/Ethyl acetate=100/1 to 1/1). tert-butyl 3-hydroxy-4-methylenepiperidine-1-carboxylate (370 g) was obtained as a white solid and the typical yield was 34.2%. ¹H NMR (400 MHz, DMSO-d₆) δ=5.22 (br d, J=3.9 Hz, 1H), 4.98 (s, 1H), 4.79 (s, 1H), 3.93-3.76 (m, 2H), 3.71 (td, J=4.3, 12.6 Hz, 1H), 2.92-2.77 (m, 1H), 2.76-2.53 (m, 1H), 2.30 (td, J=3.5, 13.4 Hz, 1H), 2.10-1.95 (m, 1H), 1.40 (s, 9H).

### Step B: tert-butyl 4-(chloromethyl)-3,6-dihydro-2H-pyridine-1-carboxylate

To the solution of tert-butyl 3-hydroxy-4-methylenepiperidine-1-carboxylate (100 g, 469 mmol, 1.00 eq) in toluene (2000 mL) was add 2,6-dimethylpyridine (55.2 g, 60.0 mL, 516 mmol, 1.10 eq) at 15° C. The mixture was cooled to 0° C., before SOCl₂ (66.9 g, 40.8 mL, 563 mmol, 1.20 eq) was added dropwise to the mixture under N₂ atmosphere, during which period the temperature was maintained below 10° C. The mixture was stirred at 110° C. for 3 hrs, before cooled to 20° C. Brine (2×600 mL) was added and the resulting mixture was stirred at 20° C. for 30 min. The organic phase was separated, before saturated NaHCO₃ solution (600 mL) was added portion-wise at 15° C. The organic phase was separated, washed with brine (1000 mL), dried over anhydrous Na₂SO₄, filtered and concentrated in vacuo. tert-Butyl 4-(chloromethyl)-3,6-dihydro-2H-pyridine-1-carboxylate (200 g) was obtained as a red oil and the typical yield was 49.0%. ¹H NMR (400 MHz, CDCl₃-d) δ=5.72 (br s, 1H), 3.98 (s, 2H), 3.88 (br s, 2H), 3.49 (br t, J=5.6 Hz, 2H), 2.17 (br s, 2H), 1.43 (s, 9H).

### Intermediate B2: (S)-3-(6-oxo-6,8-dihydro-2H,7H-spiro[furo[2,3-e]isoindole-3,4′-piperidin]-7-yl)piperidine-2,6-dione

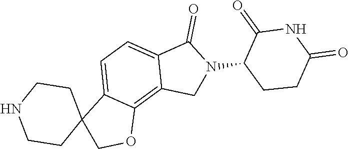 [SMILES: O=C1CC[C@H](N2Cc3c(ccc4c3OCC43CCNCC3)C2=O)C(=O)N1]

### Step A: Methyl 4-bromo-2-formyl-3-hydroxybenzoate

The solution of methyl 4-bromo-3-hydroxybenzoate (200 g, 865 mmol, 1.00 eq) in TFA (2.0 L) was added HMTA (485 g, 3.46 mol, 4.00 eq) at 20° C., before the resulting mixture was stirred at 125° C. for 12 hrs. The mixture was cooled to 20° C., quenched with 2N HCl solution (5 V), and yellow precipitate was observed. The mixture was stirred for 10 min, before additional H₂O (5 V) was added, and the reaction mixture was stirred for further 1 hr. The mixture was filtered, and the filter cake was dissolved in DCM (2.0 L), filtered over celite, dried over anhydrous Na₂SO₄ and concentrated in vacuo. Methyl 4-bromo-2-formyl-3-hydroxybenzoate (144 g) was obtained as a gray solid, and the typical yield was 64.2%. ¹HNMR (400 MHz, DMSO-d₆) δ=12.06 (br s, 1H), 10.38 (s, 1H), 8.00 (d, J=8.2 Hz, 1H), 7.30 (d, J=8.2 Hz, 1H), 3.87 (s, 3H).

### Step B: (S)-tert-butyl 5-amino-4-(5-bromo-4-hydroxy-1-oxoisoindolin-2-yl)-5-oxopentanoate

To the suspension of methyl 4-bromo-2-formyl-3-hydroxybenzoate (17.3 g, 72.4 mmol, 1.05 eq, HCl salt) in MeOH (300 mL) was added DIPEA (9.37 g, 72.4 mmol, 12.6 mL, 1.05 eq), compound 2, (17.8 g, 69.0 mmol, 1.00 eq) and AcOH (6.22 g, 103 mmol, 5.92 mL, 1.50 eq) at 20° C. and stirred for 1.5 hrs, before NaBH₃CN (8.67 g, 138 mmol, 2.00 eq) was added portion-wise at 20° C., and the resulting mixture was stirred at 20° C. for 3 hrs. The mixture was quenched by H₂O (200 mL) at 20° C. and concentrated under reduced pressure. The solvent residue was then extracted with EtOAc (3×150 mL), and the combined organic layer was washed with brine (2×200 mL), dried over anhydrous Na₂SO₄, filtered, and concentrated in vacuo. The crude product was purified by silica gel column chromatography (Petroleum ether/Ethyl acetate=1/1 to 100% Ethyl acetate). (S)-tert-butyl 5-amino-4-(5-bromo-4-hydroxy-1-oxoisoindolin-2-yl)-5-oxopentanoate (23.0 g) was obtained as a yellow solid and the typical yield was 78.4%. ¹H NMR (400 MHz, DMSO-d6) δ=10.44 (s, 1H), 7.67-7.55 (m, 2H), 7.20 (s, 1H), 7.11 (d, J=7.9 Hz, 1H), 4.76-4.67 (m, 1H), 4.58 (d, J=17.9 Hz, 1H), 4.39 (d, J=17.9 Hz, 1H), 2.23-2.07 (m, 3H), 2.03-1.91 (m, 1H), 1.32 (s, 9H).

### Step C: (S)-tert-butyl 4-(((2-(1-amino-5-(tert-butoxy)-1,5-dioxopentan-2-yl)-5-bromo-1-oxoisoindolin-4-yl)oxy)methyl)-5,6-dihydropyridine-1(2H)-carboxylate

To the solution of tert-butyl 4-(chloromethyl)-3,6-dihydro-2H-pyridine-1-carboxylate (150 g, 363 mmol, 1.00 eq) in MeCN (2000 mL) was added K₂CO₃ (150.49 g, 1.09 mmol, 3.00 eq), NaI (5.44 g, 0.36 mmol, 0.10 eq) and (S)-tert-butyl 5-amino-4-(5-bromo-4-hydroxy-1-oxoisoindolin-2-yl)-5-oxopentanoate (136 g, 472 mmol, 1.30 eq, 80% purity) at 20° C. The reaction mixture was stirred at 60° C. for 12 hrs, before being cooled to 20° C. again. The resulting mixture was filtered, and filter cake was washed with DCM (2×500 mL). The filtrate was concentrated in vacuo, and the crude product was purified by silica gel column chromatography (Petroleum ether/Ethyl acetate=100/1 to 1/1). (S)-tert-butyl 4-(((2-(1-amino-5-(tert-butoxy)-1,5-dioxopentan-2-yl)-5-bromo-1-oxoisoindolin-4-yl)oxy)methyl)-5,6-dihydropyridine-1(2H)-carboxylate (337 g) was obtained as a red solid, and the typical yield was 72.4%. ¹H NMR (400 MHz, CDCl₃-d) δ=7.67 (d, J=8.0 Hz, 1H), 7.44 (d, J=8.0 Hz, 1H), 6.33 (br s, 1H), 5.86 (br s, 1H), 5.48 (br s, 1H), 4.90 (dd, J=6.3, 8.6 Hz, 1H), 4.66-4.59 (m, 1H), 4.53 (s, 1H), 4.50 (br s, 2H), 3.98 (br s, 2H), 3.60 (br t, J=5.5 Hz, 2H), 2.43-2.10 (m, 7H), 1.49 (s, 9H), 1.41 (s, 9H).

### Step D: tert-butyl 7-[(1S)-4-tert-butoxy-1-carbamoyl-4-oxo-butyl]-6-oxo-spiro[2,8-dihydrofuro[2,3-e]isoindole-3,4′-piperidine]-1′-carboxylate

To the solution of (S)-tert-butyl 4-(((2-(1-amino-5-(tert-butoxy)-1,5-dioxopentan-2-yl)-5-bromo-1-oxoisoindolin-4-yl)oxy)methyl)-5,6-dihydropyridine-1(2H)-carboxylate (125 g, 205 mmol, 1.00 eq) in toluene (1500 mL) was added AIBN (5.06 g, 0.03 mmol, 0.15 eq) and Bu₃SnH (270 mL, 1.02 mmol, 4.98 eq) at 20° C. The reaction mixture was stirred at 110° C. for 12 hrs, before being cooled to 20° C. Saturated KF solution (1000 mL) was added and the resulting mixture was stirred at 20° C. for further 2 hrs. The mixture was filtered and the filter cake was washed by EtOAc (2×500 mL). The organic phase was separated, and the aqueous phase was extracted with ethyl acetate (3×500 mL). The combined organic phase was washed with brine (500 mL), dried over anhydrous Na₂SO₄, filtered and concentrated in vacuo. The crude product was purified by silica gel column chromatography (Petroleum ether/Ethyl acetate=100/1 to 1/1). tert-Butyl 7-[(1S)-4-tert-butoxy-1-carbamoyl-4-oxo-butyl]-6-oxo-spiro[2,8-dihydrofuro[2,3-e]isoindole-3,4′-piperidine]-1′-carboxylate (160 g) was obtained as a white solid, and the typical yield was 56.6%. ¹H NMR (400 MHz, CDCl₃-d) δ=7.40 (d, J=7.6 Hz, 1H), 7.20 (d, J=7.6 Hz, 1H), 6.41 (br s, 1H), 5.61 (br s, 1H), 4.92-4.85 (m, 1H), 4.55-4.49 (m, 2H), 4.12 (q, J=7.1 Hz, 3H), 2.88 (br t, J=12.0 Hz, 2H), 2.40-2.09 (m, 5H), 1.94-1.82 (m, 2H), 1.77-1.68 (m, 2H), 1.50-1.47 (m, 9H), 1.42-1.40 (m, 9H).

### Step E: (3S)-3-(6-oxospiro[2,8-dihydrofuro[2,3-e]isoindole-3,4′-piperidine]-7-yl)piperidine-2,6-dione benzenesulfonate

The solution of anhydrous benzene sulfonic acid (19.6 g, 124 mmol, 2.00 eq) in MeCN (400 mL) was heated to 100° C., before a solution of tert-butyl 7-[(1S)-4-tert-butoxy-1-carbamoyl-4-oxo-butyl]-6-oxo-spiro[2,8-dihydrofuro[2,3-e]isoindole-3,4′-piperidine]-1′-carboxylate (47.0 g, 62.1 mmol, 1.00 eq, 70% purity) in MeCN (100 mL) was added dropwise to the mixture. The mixture was stirred at 100° C. for 12 hrs, before being cooled to 20° C. The mixture was filtered, and the filter cake was dried under reduce pressure. The title compound (37.0 g) was obtained as a white solid, and the typical yield was 92.8%. ¹H NMR (400 MHz, D₂O-d₂) δ=7.75 (br d, J=7.4 Hz, 2H), 7.55-7.36 (m, 5H), 5.11 (br dd, J=5.2, 13.4 Hz, 1H), 4.64 (s, 2H), 4.53-4.35 (m, 2H), 3.49 (br d, J=13.2 Hz, 2H), 3.20-3.06 (m, 2H), 2.99-2.78 (m, 2H), 2.48 (dq, J=5.3, 13.1 Hz, 1H), 2.25-2.08 (m, 3H), 2.05-1.93 (m, 5H).

### Step F: (3S)-3-(6-oxospiro[2,8-dihydrofuro[2,3-e]isoindole-3,4′-piperidine]-7-yl)piperidine-2,6-dione hydrochloric acid

The solution of (3S)-3-(6-oxospiro[2,8-dihydrofuro[2,3-e]isoindole-3,4′-piperidine]-7-yl)piperidine-2,6-dione benzenesulfonate (37 g) in HCl/dioxane (4 M, 370 mL) was stirred at 20° C. for 12 hrs, before the mixture was filtered and the cake was washed with MeCN (2×200 mL). The filtered cake was dried under reduced pressure. The title compound (24.0 g) was obtained as a red solid, and the typical yield was 80.7%. ¹H NMR (400 MHz, D₂O-d₂) δ=7.48-7.36 (m, 2H), 5.12 (dd, J=5.3, 13.3 Hz, 1H), 4.69-4.61 (m, 2H), 4.53-4.37 (m, 2H), 3.51 (br dd, J=3.4, 13.3 Hz, 2H), 3.22-3.06 (m, 2H), 2.98-2.80 (m, 2H), 2.56-2.43 (m, 1H), 2.29-2.08 (m, 3H), 2.05-1.90 (m, 2H).

### Intermediate B3: (S)-3-(6-oxo-6,8-dihydro-2H,7H-spiro[furo[2,3-e]isoindole-3,4′-piperidin]-7-yl-2,2-d2)piperidine-2,6-dione

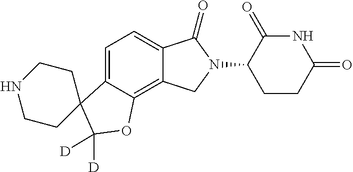 [SMILES: [2H]C1([2H])Oc2c(ccc3c2CN([C@H]2CCC(=O)NC2=O)C3=O)C12CCNCC2]

### Step A: 1-(tert-butyl) 4-methyl 3,6-dihydropyridine-1,4(2H)-dicarboxylate

To a solution of tert-butyl 4-(((trifluoromethyl)sulfonyl)oxy)-3,6-dihydropyridine-1(2H)-carboxylate (10.0 g, 30.2 mmol, 1.0 eq) in MeOH (150.0 mL) were added DIPEA (39.0 g, 52.6 mL, 302 mmol, 10.0 eq) and Pd(dppf)Cl₂ (2.21 g, 3.02 mmol, 0.1 eq). The resulting mixture was stirred under CO (1 atm) at 70° C. for 1 h. After cooled to room temperature, the mixture was filtered and the filtrate was concentrated under reduced pressure. The residue was diluted with H₂O (30.0 mL) and extracted with EA (60 mL×3). The organic layer was washed with brine (50 mL×3), dried over anhydrous Na₂SO₄, filtered and concentrated under reduced pressure. The residue was purified by flash column chromatography (ethyl acetate in petroleum ether, from 0% to 19%) to give 1-(tert-butyl) 4-methyl 3,6-dihydropyridine-1,4(2H)-dicarboxylate (5.80 g, yield 79%) as a yellow oil. LC-MS (ESI): mass calcd. for C₁₂H₁₉NO₄, 241.29; m/z found, 186.7 [M−55]⁺. ¹H NMR (400 MHz, DMSO-d₆) δ 6.86 (s, 1H), 4.00 (d, J=2.4 Hz, 2H), 3.68 (s, 3H), 3.42 (t, J=5.6 Hz, 2H), 2.28-2.25 (m, 2H), 1.41 (s, 9H).

### Step B: tert-butyl 4-(hydroxymethyl-d2)-3,6-dihydropyridine-1(2H)-carboxylate

To a solution of 1-(tert-butyl) 4-methyl 3,6-dihydropyridine-1,4(2H)-dicarboxylate (2.10 g, 8.70 mmol, 1.0 eq) in anhydrous THF (50.0 mL) was added LiAlD₄ (402 mg, 9.57 mmol, 1.1 eq) at 0° C. in portions. The reaction mixture was stirred at 0° C. for 1 h. Na₂SO₄·10H₂O (5 g) was slowly added to above solution, filtered and the filtrate was concentrated under reduced pressure. The residue was purified by flash column chromatography (ethyl acetate in petroleum ether, from 0% to 70%) to give tert-butyl 4-(hydroxymethyl-d2)-3,6-dihydropyridine-1(2H)-carboxylate (700 mg, yield 37%) as a yellow oil. LC-MS (ESI): mass calcd. for C₁₁H₁₇D₂NO₃, 215.29; m/z found, no MS signal. ¹H NMR (400 MHz, DMSO-d₆) δ 5.56 (s, 1H), 4.71 (s, 1H), 3.80 (s, 2H), 3.39 (t, J=5.6 Hz, 2H), 2.02-1.92 (m, 2H), 1.39 (s, 9H).

### Step C: tert-butyl 4-(bromomethyl-d2)-3,6-dihydropyridine-1(2H)-carboxylate

To a solution of tert-butyl 4-(hydroxymethyl-d2)-3,6-dihydropyridine-1(2H)-carboxylate (1.90 g, 8.83 mmol, 1.0 eq) and Triphenylphosphine (3.47 g, 13.2 mmol, 1.5 eq) in DCM (50.00 mL) was added CBr₄ (4.39 g, 13.2 mmol, 1.5 eq) under N₂ in portions. The reaction mixture was stirred at 0° C. for 5 h. After evaporation, The residue was purified by flash column chromatography (ethyl acetate in petroleum ether, from 0% to 19%) to give tert-butyl 4-(bromomethyl-d2)-3,6-dihydropyridine-1(2H)-carboxylate (1.60 g, yield 65%) as a yellow oil. LC-MS (ESI): mass calcd. for C₁₁H₁₆D₂BrNO₂, 278.19; m/z found, 224.0 [M−55]⁺.

### Step D: tert-butyl 4-(((5-bromo-1-oxo-1,3-dihydroisobenzofuran-4-yl)oxy)methyl-d2)-3,6-dihydropyridine-1(2H)-carboxylate

A mixture of tert-butyl 4-(bromomethyl-d2)-3,6-dihydropyridine-1(2H)-carboxylate (2.00 g, 7.19 mmol, 1.0 eq), 5-bromo-4-hydroxyisobenzofuran-1(3H)-one (1.65 g, 7.19 mmol, 1.0 eq), and K₂CO₃ (1.99 g, 14.4 mmol, 2.0 eq) in DMF (30.0 mL) was stirred at 50° C. for 2 h. After cooled to room temperature, the mixture was dissolved in EA (100 mL), washed with brine (100 mL×3), dried over anhydrous sodium sulfate, filtered, concentrated under reduced pressure. The residue was purified by flash column chromatography (ethyl acetate in petroleum ether, from 0% to 40%) to give tert-butyl 4-(((5-bromo-1-oxo-1,3-dihydroisobenzofuran-4-yl)oxy)methyl-d2)-3,6-dihydropyridine-1(2H)-carboxylate (2.30 g, yield 75%) as a yellow oil. LC-MS (ESI): mass calcd. for C₁₉H₂₀D₂BrNO₅, 426.30; m/z found, 450.1 [M+Na]⁺. ¹H NMR (400 MHz, DMSO-d₆) δ 7.89 (d, J=8.0 Hz, 1H), 7.51 (d, J=8.0 Hz, 1H), 5.95 (s, 1H), 5.72 (s, 2H), 3.93 (s, 2H), 3.53 (t, J=5.6 Hz, 2H), 2.28 (d, J=1.8 Hz, 2H), 1.47 (s, 9H).

### Step E: tert-butyl 6-oxo-6,8-dihydro-2H-spiro[benzo[2,1-b:3,4-c′]difuran-3,4′-piperidine]-1′-carboxylate-2,2-d₂

To a solution of tert-butyl 4-(((5-bromo-1-oxo-1,3-dihydroisobenzofuran-4-yl)oxy)methyl-d2)-3,6-dihydropyridine-1(2H)-carboxylate (2.20 g, 5.16 mmol, 1.0 eq) and AIBN (254 mg, 1.55 mmol, 0.3 eq) in Toluene (30.0 mL) was added Tributyltin hydride (9.01 g, 8.38 mL, 31.0 mmol, 6.0 eq). The reaction mixture was stirred in a sealed tube at 120° C. for 16 h. After cooled to room temperature, the mixture was quenched by aqueous KF solution (30 mL) and the mixture was stirred for 2 h. After filtration, the filtrate was extracted with EA (50 mL×3). The organic phase was washed with brine (30 mL×3), dried over anhydrous Na₂SO₄, filtered and concentrated under reduced pressure. The residue was purified by flash column chromatography (ethyl acetate in petroleum ether, from 0% to 33%) to give tert-butyl 6-oxo-6,8-dihydro-2H-spiro[benzo[2,1-b:3,4-c′]difuran-3,4′-piperidine]-1′-carboxylate-2,2-d₂ (1.50 g, yield 83%) as a white solid. LC-MS (ESI): mass calcd. for C₁₉H₂₁D₂NO₅, 347.41; m/z found, 292.2 [M−55]⁺. ¹H NMR (400 MHz, DMSO-d₆) δ 7.52 (d, J=7.6 Hz, 1H), 7.38 (d, J=7.6 Hz, 1H), 5.36 (s, 2H), 3.95 (d, J=12.2 Hz, 2H), 2.91-2.87 (s, 2H), 1.85-1.77 (m, 2H), 1.71-1.67 (m, 2H), 1.42 (s, 9H).

### Step F: 1′-(tert-butoxycarbonyl)-7-(hydroxymethyl)-2H-spiro[benzofuran-3,4′-piperidine]-6-carboxylic-2,2-d₂ acid

To a solution of tert-butyl 6-oxo-6,8-dihydro-2H-spiro[benzo[2,1-b:3,4-c′]difuran-3,4′-piperidine]-1′-carboxylate-2,2-d₂ (600 mg, 1.73 mmol, 1.0 eq) in THF (12.0 mL), MeOH (12.0 mL), and H₂O (4.00 mL) was added NaOH (138 mg, 3.45 mmol, 2.0 eq). The reaction mixture was stirred at 40° C. for 1 h. After cooled to room temperature, the reaction mixture was diluted with EA (40 mL), adjusted to pH=4-5 with aqueous HCl solution (3 N), and extracted with EA (50 mL×4). The organic layer was washed with brine (50 mL×2), dried over anhydrous sodium sulfate, filtered, and concentrated to 1′-(tert-butoxycarbonyl)-7-(hydroxymethyl)-2H-spiro[benzofuran-3,4′-piperidine]-6-carboxylic-2,2-d₂ acid (600 mg, yield 95%) as a colorless oil. The crude product was directly used in the next step without purification. LC-MS (ESI): mass calcd. for C₁₉H₂₃D₂NO₆, 365.42; m/z found, 364.3 [M−H]⁻.

### Step G: tert-butyl 8-hydroxy-6-oxo-6,8-dihydro-2H-spiro[benzo[2,1-b:3,4-c′]difuran-3,4′-piperidine]-1′-carboxylate-2,2-d₂

To a solution of 1′-(tert-butoxycarbonyl)-7-(hydroxymethyl)-2H-spiro[benzofuran-3,4′-piperidine]-6-carboxylic-2,2-d₂ acid (1.50 g, 4.10 mmol, 1.0 eq) in DCM (20.0 mL) was added active manganese dioxide (7.14 g, 82.1 mmol, 20.0 eq) at 25° C. the reaction mixture was stirred at 25° C. for 4 h. After filtration via a short silica gel column, the filtrate is collected and concentrated under reduced pressure to tert-butyl 8-hydroxy-6-oxo-6,8-dihydro-2H-spiro[benzo[2,1-b:3,4-c′]difuran-3,4′-piperidine]-1′-carboxylate-2,2-d₂ (760 mg, yield 52%) as a colorless oil. The crude product was directly used in next step without purification. LC-MS (ESI): mass calcd. for C₁₉H₂₁D₂NO₆, 363.41; m/z found, 364.3 [M+H]⁺. ¹H NMR (400 MHz, DMSO-d₆) δ 8.09 (s, 1H), 7.55 (d, J=7.6 Hz, 1H), 7.33 (d, J=7.6 Hz, 1H), 6.65 (s, 1H), 3.95 (d, J=12.2 Hz, 2H), 2.89 (s, 2H), 1.83-1.78 (m, 2H), 1.70-1.67 (m, 2H), 1.43 (s, 9H).

### Step H: tert-butyl (S)-7-(1-amino-5-(tert-butoxy)-1,5-dioxopentan-2-yl)-6-oxo-7,8-dihydro-2H,6H-spiro[furo[2,3-e]isoindole-3,4′-piperidine]-1′-carboxylate-2,2-d₂

To a solution of tert-butyl 8-hydroxy-6-oxo-6,8-dihydro-2H-spiro[benzo[2,1-b:3,4-c′]difuran-3,4′-piperidine]-1′-carboxylate-2,2-d₂ (350 mg, 963 μmol, 1.0 eq) and tert-butyl (S)-4,5-diamino-5-oxopentanoate hydrochloride (460 mg, 1.93 mmol, 2.0 eq) in DMF (10.0 mL) was added Acetic acid (578 mg, 554 μL, 9.63 mmol, 10.0 eq). The reaction mixture was stirred at 40° C. for 2 h, then Sodium triacetoxyborohydride (4.08 g, 19.3 mmol, 20.0 eq). The mixture was stirred at 40° C. for 16 h. After cooled to room temperature, the mixture was dissolved in EA (40 mL), washed with brine (30 mL×3), dried over anhydrous sodium sulfate, filtered and concentrated under reduced pressure. The residue was purified by flash column chromatography on silica gel (ethyl acetate in petroleum ether, from 0% to 90%) to afford tert-butyl (S)-7-(1-amino-5-(tert-butoxy)-1,5-dioxopentan-2-yl)-6-oxo-7,8-dihydro-2H,6H-spiro[furo[2,3-e]isoindole-3,4′-piperidine]-1′-carboxylate-2,2-d2 (300 mg, yield 58%) as a white solid. LC-MS (ESI): mass calcd. for C₂₈H₃₇D₂N₃O₇, 531.65; m/z found, 532.2 [M+H]⁺. ¹H NMR (400 MHz, DMSO-d₆) δ 7.60 (s, 1H), 7.45 (d, J=7.6 Hz, 1H), 7.30 (d, J=7.6 Hz, 1H), 7.23 (s, 1H), 4.77-4.73 (m, 1H), 4.56 (d, J=17.2 Hz, 1H), 4.45 (d, J=17.2 Hz, 1H), 4.00 (d, J=12.8 Hz, 2H), 2.96 (s, 2H), 2.23-2.20 (m, 3H), 2.09-2.02 (m, 1H), 1.97-1.84 (m, 2H), 1.82-1.74 (m, 2H), 1.49 (s, 9H), 1.38 (s, 9H).

### Step I: (S)-3-(6-oxo-6,8-dihydro-2H,7H-spiro[furo[2,3-e]isoindole-3,4′-piperidin]-7-yl-2,2-d2)piperidine-2,6-dione benzenesulfonate

To a solution of tert-butyl (S)-7-(1-amino-5-(tert-butoxy)-1,5-dioxopentan-2-yl)-6-oxo-7,8-dihydro-2H,6H-spiro[furo[2,3-e]isoindole-3,4′-piperidine]-1′-carboxylate-2,2-d₂ (350 mg, 660 μmol, 1.0 eq) in MeCN (10.0 mL) was added anhydrous benzenesulfonic acid (313 mg, 1.98 mmol, 3.0 eq) and the reaction mixture was stirred at 90° C. for 7 h. After cooled to room temperature, the mixture was concentrated under reduced pressure and the residue was slurred with acetonitrile (10 mL) to afford (S)-3-(6-oxo-6,8-dihydro-2H,7H-spiro[furo[2,3-e]isoindole-3,4′-piperidin]-7-yl-2,2-d2)piperidine-2,6-dione benzenesulfonate (300 mg, yield 88%) as a yellow solid. The crude product was directly used in next step without further purification. LC-MS (ESI): mass calcd. for C₂₅H₂₅D₂N₃O₇S, 515.58; m/z found, 358.3 [M+H]⁺.

### Intermediate B4: tert-butyl (S)-5-amino-4-(5-fluoro-6-oxo-6,8-dihydro-2H,7H-spiro[furo[2,3-e]isoindole-3,4′-piperidin]-7-yl)-5-oxopentanoate

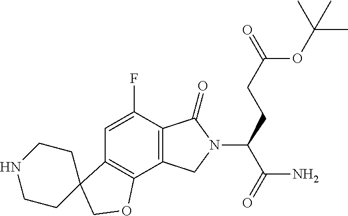 [SMILES: CC(C)(C)OC(=O)CC[C@@H](C(N)=O)N1Cc2c3c(cc(F)c2C1=O)C1(CCNCC1)CO3]

### Step A: 4-bromo-2-fluoro-5-hydroxybenzoic acid

To a solution of compound 2-fluoro-5-hydroxybenzoic acid (10.0 g, 1.0 eq.) in MeCN (100 mL) was added TsOH (10.1 g, 1.0 eq.) at rt. The mixture was stirred for 10 min, then added NBS (11.4 g, 1.1 eq.) in MeCN (50 mL) via dropwise during 20 min. The reaction mixture was stirred overnight at rt, and concentrated to afford the title compound (15.0 g) as a crude, which was used in next step without further purification.

### Step B: methyl 4-bromo-2-fluoro-5-hydroxybenzoate

To a solution of 4-bromo-2-fluoro-5-hydroxybenzoic acid (crude, 15.0 g, 1.0 eq.) in MeOH (100 mL) was added 2,2-Dimethoxypropane (18.3 g, 3.0 eq.) at rt. The reaction mixture was stirred overnight at 60° C. After cooled to room temperature and concentrated. The residual was purified by flash column chromatography (PE/EA) to afford the title compound (600 mg).

### Step C: methyl 4-bromo-6-fluoro-2-formyl-3-hydroxybenzoate

To a solution of methyl 4-bromo-2-fluoro-5-hydroxybenzoate (249 mg, 1 mmol, 1 eq.) in TFA (5 mL) was added HMTA (560 mg, 4 mmol, 4 eq) at 20° C. The mixture was stirred at 125° C. for 12 h. TLC (Petroleum ether/Ethyl acetate=5/1) indicated starting materials was consumed completely and there was desired product. The mixture was quenched with 2N HCl (5 V) and a yellow solid formed. The mixture was stirred for 10 min and then additional water (5 V) was added and stirred for 1 h. The mixture was filtered. The filter cake was dissolved in DCM and filtered on celite, dried and then remove most of the solvent in vacuo. The title compound (110 mg, 0.4 mmol, 40% yield) was obtained as a gray solid.

¹H NMR (400 MHz, Chloroform-d) δ 12.28 (s, 1H), 10.08 (s, 1H), 7.65 (d, J=8.7 Hz, 1H), 4.01 (s, 3H). LC-MS (m/z): [M−H]⁺=274.99.

### Step D: tert-butyl (S)-5-amino-4-(5-bromo-7-fluoro-4-hydroxy-1-oxoisoindolin-2-yl)-5-oxopentanoate

tert-Butyl (S)-4,5-diamino-5-oxopentanoate (212 mg, 1.05 mmol, 1.05 eq, HCl) was added in MeOH (5 mL) at 20° C. Then DIEA (1.05 mmol, 1.05 eq), methyl 4-bromo-6-fluoro-2-formyl-3-hydroxybenzoate (277 mg, 1 mmol, 1 eq) and AcOH (1.5 mmol, 1.5 eq) were added to the mixture at the same temperature. After 1.5 h, NaBH₃CN (2 mmol, 2 eq) was added to the mixture in portions and stirred the mixture at 20° C. for 3 h. After the reaction completed, the reaction mixture was quenched by addition H₂O at 20° C., and then concentrated under reduced pressure to remove MeOH. Then the mixture was extracted with EtOAc. The combined organic layers were washed with brine (200 mL×2), dried over Na₂SO₄, filtered and concentrated under reduced pressure to give a residue. The residue was purified by column chromatography (SiO₂, Petroleum ether/Ethyl acetate=1/1 to Ethyl acetate). The title compound (293 mg, 0.68 mmol, 65% yield) was obtained as a yellow solid.

### Step E: benzyl (S)-4-(((2-(1-amino-5-(tert-butoxy)-1,5-dioxopentan-2-yl)-5-bromo-6-fluoro-1-oxoisoindolin-4-yl)oxy)methyl)-3,6-dihydropyridine-1(2H)-carboxylate

tert-Butyl (S)-5-amino-4-(5-bromo-7-fluoro-4-hydroxy-1-oxoisoindolin-2-yl)-5-oxopentanoate (202 mg, 0.47 mmol, 1 eq), benzyl 4-(chloromethyl)-3,6-dihydropyridine-1(2H)-carboxylate (280 mg, 0.49 mmol, 1.05 eq) and K₂CO₃ (194 mg, 1.41 mmol, 3 eq) were added in DMF (5 mL) at 20° C. Then the mixture was stirred at 60° C. for 12 h. After the reaction completed, the mixture was concentrated under reduced pressure to give a residue, which was added water (20 mL). The product was extracted with DCM (20 mL×3). The organic layers were dried over Na₂SO₄ and concentrated under reduced pressure to give a residue. The residue was purified by column chromatography on silica gel to afford the title compound as a yellow solid (194 mg, 60% yield).

### Step F: benzyl (S)-7-(1-amino-5-(tert-butoxy)-1,5-dioxopentan-2-yl)-5-fluoro-6-oxo-7,8-dihydro-2H,6H-spiro[furo[2,3-e]isoindole-3,4′-piperidine]-1′-carboxylate

Benzyl (S)-4-(((2-(1-amino-5-(tert-butoxy)-1,5-dioxopentan-2-yl)-5-bromo-6-fluoro-1-oxoisoindolin-4-yl)oxy)methyl)-3,6-dihydropyridine-1(2H)-carboxylate (330 mg, 0.5 mmol, 1 eq), Bu₃SnH (614 mg, 2 mmol) and AIBN (8.2 mg, 0.05 mmol, 0.1 eq) were added in toluene (5 mL) at 20° C. and then stirred the mixture at 110° C. for 12 h. After the reaction completed, the mixture was quenched by addition saturated potassium fluoride solution and stirred for 1 h. The product was extracted with EA. The organic layers were dried over Na₂SO₄ and concentrated under reduced pressure to give a residue. The residue was purified by column chromatography on silica gel to afford the title compound as a yellow solid (84 mg, 29% yield).

¹H NMR (400 MHz, CDCl₃) δ7.38-7.31 (m, 5H), 6.84 (d, J=8.4 Hz, 1H), 6.40 (s, 1H), 5.35 (s, 1H), 5.16 (s, 2H), 4.85 (dd, J=8.9, 6.1 Hz, 1H), 4.52 (s, 2H), 4.48 (d, J=17.2 Hz, 1H), 4.36 (d, J=17.2 Hz, 1H), 4.20 (brs, 2H), 4.12 (q, J=7.1 Hz, 1H), 2.94 (m, 2H), 2.37-2.16 (m, 6H), 1.76 (m, 2H), 1.42 (s, 9H).

### Step G: tert-butyl (S)-5-amino-4-(5-fluoro-6-oxo-6,8-dihydro-2H,7H-spiro[furo[2,3-e]isoindole-3,4′-piperidin]-7-yl)-5-oxopentanoate

To a 100 mL flask equipped with a magnetic stirring bar was added benzyl (S)-7-(1-amino-5-(tert-butoxy)-1,5-dioxopentan-2-yl)-5-fluoro-6-oxo-7,8-dihydro-2H,6H-spiro[furo[2,3-e]isoindole-3,4′-piperidine]-1′-carboxylate (200 mg), MeOH (10 mL), and then added 10% Pd/C (20 mg). Followed by flushing flask with hydrogen, a balloon with hydrogen was attached and the reaction mixture was stirred for 2 h. Upon full consumption of the starting material by TLC monitoring (DCM:MeOH=10:1), the reaction mixture was filtered through celite and washed through with additional MeOH. Solvent was removed and the residual title compound (120 mg) as a white solid was used directly in the next step.

### Intermediate B5: (S)-3-(5-chloro-6-oxo-6,8-dihydro-2H,7H-spiro[furo[2,3-e]isoindole-3,4′-piperidin]-7-yl)piperidine-2,6-dione

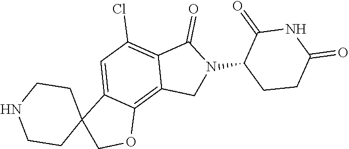 [SMILES: O=C1CC[C@H](N2Cc3c4c(cc(Cl)c3C2=O)C2(CCNCC2)CO4)C(=O)N1]

### Step A: tert-butyl (S)-7-(2,6-dioxopiperidin-3-yl)-6-oxo-7,8-dihydro-2H,6H-spiro[furo[2,3-e]isoindole-3,4′-piperidine]-1′-carboxylate

To a mixture of (S)-3-(6-oxo-6,8-dihydro-2H,7H-spiro[furo[2,3-e]isoindole-3,4′-piperidin]-7-yl)piperidine-2,6-dione (100 mg, 0.28 mmol, 1.0 eq) and TEA (85 mg, 0.84 mmol, 3 eq) in dry DCM (10 mL) was slowly added Di-tert-butyl dicarbonate (123 mg, 0.56 mmol, 2.0 eq). The mixture was stirred under N₂ atmosphere at room temperature for 5 h. The reaction mixture was quenched with water (10 mL) and exacted with EtOAc (15 mL×3). The organic layer was washed with brine (20 mL×3), dried over anhydrous Na₂SO₄, filtered and concentrated under reduced pressure. The residue was purified by flash column chromatography on silica gel (ethyl acetate in petroleum ether, from 0% to 50%) to afford tert-butyl (S)-7-(2,6-dioxopiperidin-3-yl)-6-oxo-7,8-dihydro-2H,6H-spiro[furo[2,3-e]isoindole-3,4′-piperidine]-1′-carboxylate (120 mg, yield 94%) as a light yellow solid. LC-MS (ESI): mass calced for C₂₄H₂₉N₃O₆: 455; m/z found, 456.2 [M+H]⁺. ¹H NMR (400 MHz, DMSO-d₆) δ 10.97 (s, 1H), 7.42 (d, J=7.6 Hz, 1H), 7.27 (d, J=7.6 Hz, 1H), 5.11-5.06 (m, 1H), 4.65-4.50 (m, 2H), 4.39 (d, J=17.2 Hz, 1H), 4.22 (d, J=17.2 Hz, 1H), 3.94 (d, J=11.2 Hz, 2H), 3.07-2.76 (m, 3H), 2.62-2.56 (m, 1H), 2.42-2.33 (m, 1H), 2.05-1.91 (m, 1H), 1.88-1.76 (m, 2H), 1.70 (d, J=10.6 Hz, 2H), 1.43 (s, 9H).

### Step B: tert-butyl (S)-5-chloro-7-(2,6-dioxopiperidin-3-yl)-6-oxo-7,8-dihydro-2H,6H-spiro[furo[2,3-e]isoindole-3,4′-piperidine]-1′-carboxylate

To a solution of tert-butyl (S)-7-(2,6-dioxopiperidin-3-yl)-6-oxo-7,8-dihydro-2H,6H-spiro[furo[2,3-e]isoindole-3,4′-piperidine]-1′-carboxylate (60 mg, 132 μmol, 1.0 eq) in dry ACN (5.0 mL) was added NCS (18 mg, 132 μmol, 1.0 eq) and the mixture was stirred under N₂ atmosphere at room temperature for 6 h. The reaction mixture was quenched with water (10 mL) and exacted with EtOAc (15 mL×3). The organic layer was washed with brine (20 mL×3), dried over anhydrous Na₂SO₄, filtered and concentrated under reduced pressure. The residue was purified by flash column chromatography on silica gel (ethyl acetate in petroleum ether, from 0% to 50%) to afford tert-butyl (S)-4-chloro-7-(2,6-dioxopiperidin-3-yl)-6-oxo-7,8-dihydro-2H,6H-spiro[furo[2,3-e]isoindole-3,4′-piperidine]-1′-carboxylate (60.0 mg, yield 93%) as a light yellow solid. LC-MS (ESI): mass calced for C₂₄H₂₈ClN₃O₆ 489; m/z found, 490.2 [M+H]⁺. ¹H NMR (400 MHz, DMSO-d₆) δ 11.10 (s, 1H), 7.62 (s, 1H), 5.20-5.15 (m, 1H), 4.80-4.67 (m, 1H), 4.47 (d, J=17.4 Hz, 1H), 4.30 (d, J=17.4 Hz, 1H), 4.15-4.05 (m, 2H), 3.10-2.91 (m, 3H), 2.65-2.611 (m, 1H), 2.53-2.45 (m, 1H), 2.11-2.06 (m, 1H), 2.03-1.94 (m, 2H), 1.86-1.75 (m, 2H), 1.56 (s, 9H).

### Step C: (S)-3-(5-chloro-6-oxo-6,8-dihydro-2H,7H-spiro[furo[2,3-e]isoindole-3,4′-piperidin]-7-yl)piperidine-2,6-dione

To a solution of tert-butyl (S)-7-(2,6-dioxopiperidin-3-yl)-6-oxo-7,8-dihydro-2H,6H-spiro[furo[2,3-e]isoindole-3,4′-piperidine]-1′-carboxylate (50 mg, 110 μmol, 1.0 eq) in EA (5.0 mL) was added HCl-dioxane (4 N) (0.25 mL, 1.00 mmol, 10.0 eq) and the mixture was stirred at room temperature for 3 h. The reaction mixture was concentrated under reduced pressure to afford (S)-3-(4-chloro-6-oxo-6,8-dihydro-2H,7H-spiro[furo[2,3-e]isoindole-3,4′-piperidin]-7-yl)piperidine-2,6-dione hydrochloride (40.0 mg, yield 93%) as a light yellow solid. LC-MS (ESI): mass calced for C₁₉H₂OClN₃O₄ 389; m/z found, 390.2 [M+H]⁺.

### Intermediate B6: (S)-7-(2,6-dioxopiperidin-3-yl)-6-oxo-7,8-dihydro-2H,6H-spiro[furo[2,3-e]isoindole-3,4′-piperidine]-5-carbonitrile

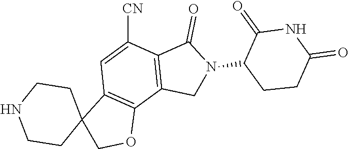

### Step A: (S)-tert-butyl 7-(1-amino-5-(tert-butoxy)-1,5-dioxopentan-2-yl)-5-cyano-6-oxo-7,8-dihydro-2H,6H-spiro[furo[2,3-e]isoindole-3,4′-piperidine]-1′-carboxylate

To a solution of tert-butyl (S)-7-(1-amino-5-(tert-butoxy)-1,5-dioxopentan-2-yl)-5-bromo-6-oxo-7,8-dihydro-2H,6H-spiro[furo[2,3-e]isoindole-3,4′-piperidine]-1′-carboxylate (200 mg, 329 μmol, 1.0 eq) in DMF (10.0 mL) was added cuprous cyanide (88.3 mg, 986 μmol, 3.0 eq) at 25° C. The reaction mixture was stirred at 140° C. for 5 h. After cooled to room temperature, the reaction mixture was quenched with water (20 mL) and extracted with DCM (30 mL×3). The organic layer was washed with brine (30 mL×4), dried over anhydrous Na₂SO₄, filtered and concentrated under reduced pressure. The residue was purified by flash column chromatography on silica gel (PE/EA=1/1) to give (S)-tert-butyl 7-(1-amino-5-(tert-butoxy)-1,5-dioxopentan-2-yl)-5-cyano-6-oxo-7,8-dihydro-2H,6H-spiro[furo[2,3-e]isoindole-3,4′-piperidine]-1′-carboxylate (160 mg, yield 88%) as a yellow solid. LC-MS (ESI): mass calced for: C₂₉H₃₈N₄O₇ 554.27; m/z found, 555.3 [M+H]⁺.

### Step B: (S)-7-(2,6-dioxopiperidin-3-yl)-6-oxo-7,8-dihydro-2H,6H-spiro[furo[2,3-e]isoindole-3,4′-piperidine]-5-carbonitrile

To a solution of (S)-tert-butyl 7-(1-amino-5-(tert-butoxy)-1,5-dioxopentan-2-yl)-5-cyano-6-oxo-7,8-dihydro-2H,6H-spiro[furo[2,3-e]isoindole-3,4′-piperidine]-1′-carboxylate (160 mg, 288 μmol, 1.0 eq) in MeCN (5.0 mL) was added anhydrous benzenesulfonic acid (151.4 mg, 288 μmol, 3.0 eq) at 0° C. The reaction mixture was stirred at 80° C. for 3 h. The reaction mixture was concentrated under reduced pressure to get crude (S)-7-(2,6-dioxopiperidin-3-yl)-6-oxo-7,8-dihydro-2H,6H-spiro[furo[2,3-e]isoindole-3,4′-piperidine]-5-carbonitrile (20.0 mg, yield 18%) as a yellow solid. The crude product was directly used in next step without further purification. LC-MS (ESI): mass calced for: C₂₀H₂₀N₄O₄ 380.15; m/z found, 381.2 [M+H]⁺.

### Intermediate B7: (S)-3-(5-methyl-6-oxo-6,8-dihydro-2H,7H-spiro[furo[2,3-e]isoindole-3,4′-piperidin]-7-yl)piperidine-2,6-dione

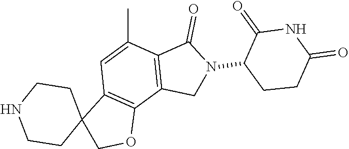

### Step A: tert-butyl (S)-7-(1-amino-5-(tert-butoxy)-1,5-dioxopentan-2-yl)-5-bromo-6-oxo-7,8-dihydro-2H,6H-spiro[furo[2,3-e]isoindole-3,4′-piperidine]-1′-carboxylate

To a solution of tert-butyl (S)-7-(1-amino-5-(tert-butoxy)-1,5-dioxopentan-2-yl)-6-oxo-7,8-dihydro-2H,6H-spiro[furo[2,3-e]isoindole-3,4′-piperidine]-1′-carboxylate (200 mg, 378 μmol, 1.0 eq) in MeCN (5.0 mL) was added NBS (67.2 mg, 378 μmol, 1.0 eq) at 25° C. The reaction mixture was stirred at room temperature for 3 h. The reaction mixture was quenched with water (10 mL) and extracted with EA (10 mL×3). The organic layer was washed with brine (10 mL), dried over Na₂SO₄, filtered and concentrated under reduced pressure. The residue was purified by flash column chromatography on silica gel (PE/EA=2/1) to give tert-butyl (S)-7-(1-amino-5-(tert-butoxy)-1,5-dioxopentan-2-yl)-5-bromo-6-oxo-7,8-dihydro-2H,6H-spiro[furo[2,3-e]isoindole-3,4′-piperidine]-1′-carboxylate (180 mg, yield 78%) as a white solid. LC-MS (ESI): mass calced for: C₂₈H₃₈BrN₃O₇ 607.2; m/z found, 608.2 [M+H]⁺.

### Step B: tert-butyl (S)-7-(1-amino-5-(tert-butoxy)-1,5-dioxopentan-2-yl)-5-methyl-6-oxo-7,8-dihydro-2H,6H-spiro[furo[2,3-e]isoindole-3,4′-piperidine]-1′-carboxylate

To a solution of tert-butyl (S)-7-(1-amino-5-(tert-butoxy)-1,5-dioxopentan-2-yl)-5-bromo-6-oxo-7,8-dihydro-2H,6H-spiro[furo[2,3-e]isoindole-3,4′-piperidine]-1′-carboxylate (200 mg, 329 μmol, 1.0 eq), Potassium carbonate (136 mg, 986 mol, 3.0 eq), and Pd(dppf)Cl₂ (48.1 mg, 65.7 μmol, 0.2 eq) in 1,4-dioxane (5.0 mL) and water (0.5 mL) was added 2,4,6-trimethyl-1,3,5,2,4,6-trioxatriborinane (82.5 mg, 657 μmol, 2.0 eq) at 25° C. The reaction mixture was stirred under N₂ at 80° C. for 2 h. After cooled to room temperature, the reaction mixture was filtered and the filtrate was diluted with water (10 mL), and extracted with EtOAc (10 mL×3). The organic layer was washed with brine (10 mL), dried over Na₂SO₄, filtered and concentrated under reduced pressure. The residue was purified by Prep-TLC (PE/EA=1/1) to afford tert-butyl (S)-7-(1-amino-5-(tert-butoxy)-1,5-dioxopentan-2-yl)-5-methyl-6-oxo-7,8-dihydro-2H,6H-spiro[furo[2,3-e]isoindole-3,4′-piperidine]-1′-carboxylate (141 mg, yield 79%) as a yellow solid. LC-MS (ESI): mass calced for: C₂₉H₄₁N₃O₇ 543.3; m/z found, 544.3 [M+H]⁺.

### Step C: (S)-3-(5-methyl-6-oxo-6,8-dihydro-2H,7H-spiro[furo[2,3-e]isoindole-3,4′-piperidin]-7-yl)piperidine-2,6-dione

To a solution of tert-butyl (S)-7-(1-amino-5-(tert-butoxy)-1,5-dioxopentan-2-yl)-5-methyl-6-oxo-7,8-dihydro-2H,6H-spiro[furo[2,3-e]isoindole-3,4′-piperidine]-1′-carboxylate (141 mg, 259 μmol, 1.0 eq) in MeCN (5.0 mL) was added benzenesulfonic acid (123 mg, 778 μmol, 3.0 eq) at 25° C. The reaction mixture was stirred at 80° C. for 2 h. The reaction mixture was concentrated under reduced pressure to get (S)-3-(5-methyl-6-oxo-6,8-dihydro-2H,7H-spiro[furo[2,3-e]isoindole-3,4′-piperidin]-7-yl)piperidine-2,6-dione benzenesulfonic acid (360 mg, yield 79%, 30% purity) as a yello oil. The crude product was directly used in next step without further purification. LC-MS (ESI): mass calced for: C₂₀H₂₃N₃O₄ 369.2; m/z found, 370.2 [M+H]⁺.

### Intermediate B8: 3-(3′-methyl-6-oxo-6,8-dihydro-2H,7H-spiro[furo[2,3-e]isoindole-3,4′-piperidin]-7-yl)piperidine-2,6-dione

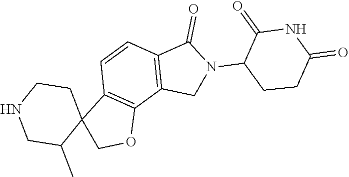 [SMILES: CC1CNCCC12COc1c2ccc2c1CN(C1CCC(=O)NC1=O)C2=O]

### Step A: tert-butyl 4-methyl-1-oxa-6-azaspiro[2.5]octane-6-carboxylate

To a solution of Trimethyl(oxo)sulfonium iodide (8.25 g, 37.5 mmol, 4.0 eq) in DMSO (10.0 mL) was added NaH (60% suspend in oil) (1.5 g, 37.5 mmol, 4.0 eq) at 0° C. and the mixture was stirred under N₂ at room temperature for 1 h. Then tert-butyl 3-methyl-4-oxopiperidine-1-carboxylate (2.00 g, 9.38 mmol, 1.0 eq) was added to above mixture and the resulting mixture was stirred under N₂ at room temperature for 16 h. The reaction mixture was quenched with ice-water (50 mL) and exacted with EA (100 mL×3). The organic layer was washed with brine (100 mL×4), dried over anhydrous Na₂SO₄, filtered and concentrated under reduced pressure. The residue was purified by flash column chromatography on silica gel (ethyl acetate in petroleum ether, from 20% to 50%) to afford tert-butyl 4-methyl-1-oxa-6-azaspiro[2.5]octane-6-carboxylate (400 mg, yield 19%) as a light yellow solid. LC-MS (ESI): mass calcd. for C₁₂H₂₁NO₃, 227.30; m/z found, 172.1 [M−55]⁺. ¹H NMR (400 MHz, DMSO-d₆) δ 3.48-3.40 (m, 3H), 3.14 (s, 1H), 2.75 (d, J=4.6 Hz, 1H), 2.55 (dd, J=12.0, 8.6 Hz, 1H), 1.73 (s, 1H), 1.52 (s, 2H), 1.41 (s, 9H), 0.75 (d, J=6.8 Hz, 3H).

### Step B: tert-butyl 4-(hydroxymethyl)-3-methyl-3,6-dihydropyridine-1(2H)-carboxylate

To a solution of tert-butyl 4-methyl-1-oxa-6-azaspiro[2.5]octane-6-carboxylate (1.00 g, 4.40 mmol, 1.0 eq) in Toluene (10.0 mL) was added Aluminum isopropoxide (2.70 g, 2.60 mL, 13.2 mmol, 3.0 eq). The mixture was stirred under N₂ at 110° C. for 16 h. After cooled to room temperature, the reaction mixture was adjusted to pH=4-5 with aqueous HCl solution (3 N) and exacted with EA (50 mL×3). The organic layer was washed with brine (20 mL×3), dried over anhydrous Na₂SO₄, filtered and concentrated under reduced pressure. The residue was purified by flash column chromatography on silica gel (ethyl acetate in petroleum ether, from 20% to 50%) to afford tert-butyl 4-(hydroxymethyl)-3-methyl-3,6-dihydropyridine-1(2H)-carboxylate (850 mg, yield 85%) as a light yellow oil. LC-MS: No MS signal under routine condition. LC-MS (ESI): mass calcd. for C₁₂H₂₁NO₃, 227.30; m/z found, no MS signal.

### Step C: tert-butyl 4-(((5-bromo-1-oxo-1,3-dihydroisobenzofuran-4-yl)oxy)methyl)-3-methyl-3,6-dihydropyridine-1(2H)-carboxylate

To a solution of 5-bromo-4-hydroxyisobenzofuran-1(3H)-one (420 mg, 1.83 mmol, 1.0 eq) in THF (20.0 mL) were added Triphenylphosphine (721 mg, 2.75 mmol, 1.5 eq) and tert-butyl 4-(hydroxymethyl)-3-methyl-3,6-dihydropyridine-1(2H)-carboxylate (417 mg, 1.83 mmol, 1.0 eq). The mixture was stirred under N₂ at 0° C. for 20 min, then DIAD (556 mg, 535 μL, 2.75 mmol, 1.5 eq) was dropwise to above mixture. The resulting mixture was stirred at room temperature overnight. After evaporation, the crude product was purified by flash column chromatography on silica gel (ethyl acetate in petroleum ether, from 0% to 20%) to afford tert-butyl 4-(((5-bromo-1-oxo-1,3-dihydroisobenzofuran-4-yl)oxy)methyl)-3-methyl-3,6-dihydropyridine-1(2H)-carboxylate (180 mg, yield 22%) as a yellow oil. LC-MS (ESI): mass calcd. for C₂₀H₂₄BrNO₅, 438.32; m/z found, 342.0 [M−55]⁺.

### Step D: tert-butyl 3′-methyl-6-oxo-6,8-dihydro-2H-spiro[benzo[2,1-b:3,4-c′]difuran-3,4′-piperidine]-1′-carboxylate

To a solution of tert-butyl 4-(((5-bromo-1-oxo-1,3-dihydroisobenzofuran-4-yl)oxy)methyl)-3-methyl-3,6-dihydropyridine-1(2H)-carboxylate (180 mg, 411 μmol, 1.0 eq) and AIBN (13.5 mg, 82.1 μmol, 0.2 eq) in Toluene (20.0 mL) was added Tributyltin hydride (478 mg, 444 μL, 1.64 mmol, 4.0 eq). The reaction was stirred under N₂ at 110° C. for 16 h. After cooled to room temperature, the mixture was quenched by saturated aqueous KF solution (30 mL) and stirred for 1 h. The reaction mixture was extracted with EA (30 mL×3). The organic phases were combined, washed with brine (30 mL×2), dried over anhydrous Na₂SO₄, filtered and concentrated under reduced pressure. The mixture was purified by flash column chromatography on silica gel (ethyl acetate in petroleum ether, from 0% to 30%) to give tert-butyl 3′-methyl-6-oxo-6,8-dihydro-2H-spiro[benzo[2,1-b:3,4-c′]difuran-3,4′-piperidine]-1′-carboxylate (120 mg, yield 81%) as a white solid. LC-MS (ESI): mass calcd. for C₂₀H₂₅NO₅, 359.42; m/z found, 304.0 [M−55]⁺. ¹H NMR (400 MHz, DMSO-d₆) δ 7.47 (d, J=7.6 Hz, 1H), 7.40 (d, J=7.6 Hz, 1H), 5.36 (d, J=2.4 Hz, 2H), 4.79 (d, J=9.8 Hz, 1H), 4.48 (d, J=9.8 Hz, 1H), 3.99-3.77 (m, 2H), 3.04-2.66 (m, 2H), 2.01 (d, J=10.8 Hz, 3H), 1.97-1.87 (m, 2H), 1.77 (d, J=13.2 Hz, 1H), 1.43 (s, 9H).

### Step E: tert-butyl 3′-methyl-6-oxo-6,8-dihydro-2H-spiro[benzo[2,1-b:3,4-c′]difuran-3,4′-piperidine]-1′-carboxylate

To a solution of tert-butyl 3′-methyl-6-oxo-6,8-dihydro-2H-spiro[benzo[2,1-b:3,4-c′]difuran-3,4′-piperidine]-1′-carboxylate (110 mg, 306 μmol, 1.0 eq) in in THF (9.00 mL), MeOH (9.00 mL), and H₂O (3.00 mL) was added NaOH (24 mg, 612 μmol, 2.0 eq). The reaction mixture was stirred at 40° C. for 1 h. After cooled to room temperature, the reaction mixture was diluted with EA (20 mL), adjusted to pH=4-5 with aqueous HCl solution (3 N), and extracted with EA (40 mL×4). The organic layer was washed with brine (20 mL×2), dried over anhydrous sodium sulfate, filtered and concentrated to get tert-butyl 3′-methyl-6-oxo-6,8-dihydro-2H-spiro[benzo[2,1-b:3,4-c′]difuran-3,4′-piperidine]-1′-carboxylate (110 mg, yield 90%) as a colorless oil. The crude product was directly used in next step without purification. LC-MS (ESI): mass calcd. for C₂₀H₂₇NO₆, 377.44; m/z found, 376.4 [M−H]⁻.

### Step F: 1′-(tert-butoxycarbonyl)-7-formyl-3′-methyl-2H-spiro[benzofuran-3,4′-piperidine]-6-carboxylic acid

A mixture of 1′-(tert-butoxycarbonyl)-7-(hydroxymethyl)-3′-methyl-2H-spiro[benzofuran-3,4′-piperidine]-6-carboxylic acid (110 mg, 291 μmol, 1.0 eq) and active Manganese dioxide (507 mg, 5.83 mmol, 20.0 eq) in DCM (20.0 mL) was stirred at room temperature for 16 h. After filtration via a short column, the filtrate is collected and concentrated under reduced pressure to afford 1′-(tert-butoxycarbonyl)-7-formyl-3′-methyl-2H-spiro[benzofuran-3,4′-piperidine]-6-carboxylic acid (100 mg, yield 91%) as a colorless oil. The crude product was directly used in next step without purification. LC-MS (ESI): mass calcd. for C₂₀H₂₅NO₆, 375.42; m/z found, 376.1 [M+H]⁺.

### Step G: tert-butyl 7-(2,6-dioxopiperidin-3-yl)-3′-methyl-6-oxo-7,8-dihydro-2H,6H-spiro[furo[2,3-e]isoindole-3,4′-piperidine]-1′-carboxylate

To a solution of 1′-(tert-butoxycarbonyl)-7-formyl-3′-methyl-2H-spiro[benzofuran-3,4′-piperidine]-6-carboxylic acid (110 mg, 293 μmol, 1.0 eq) and 3-aminopiperidine-2,6-dione hydrochloride (96.5 mg, 586 μmol, 2.0 eq) in DMF (10.0 mL) was added Acetic acid (0.52 g, 0.50 mL, 8.7 mmol, 30.0 eq) and the reaction mixture was stirred at 40° C. for 2 h. Then Sodium triacetoxyborohydride (186 mg, 879 μmol, 3.0 eq) was added to above mixture and the mixture was stirred at 40° C. for 16 h. After cooled to room temperature, the mixture was dissolve in EA (40 mL), washed with brine (30 mL×3), dried over anhydrous sodium sulfate, filtered and concentrated under reduced pressure. The residue was purified by Prep-TLC (DCM/MeOH=10/1) to afford tert-butyl 7-(2,6-dioxopiperidin-3-yl)-3′-methyl-6-oxo-7,8-dihydro-2H,6H-spiro[furo[2,3-e]isoindole-3,4′-piperidine]-1′-carboxylate (80.0 mg, yield 58%) as a gray solid. LC-MS (ESI): mass calcd. for C₂₅H₃₁N₃O₆, 469.54; m/z found, 470.2 [M+H]⁺.

### Step H: 3-(3′-methyl-6-oxo-6,8-dihydro-2H,7H-spiro[furo[2,3-e]isoindole-3,4′-piperidin]-7-yl)piperidine-2,6-dione

To a solution of tert-butyl 7-(2,6-dioxopiperidin-3-yl)-3′-methyl-6-oxo-7,8-dihydro-2H,6H-spiro[furo[2,3-e]isoindole-3,4′-piperidine]-1′-carboxylate (70.0 mg, 149 μmol, 1.0 eq) in DCM (3 mL) was added dropwise HCl-dioxane (4 N) (8.0 mL, 32 mmol, 10.4 eq) at room temperature. The reaction mixture was stirred at room temperature for 2 h. The reaction mixture was concentrated under reduced pressure to give 3-(3′-methyl-6-oxo-6,8-dihydro-2H,7H-spiro[furo[2,3-e]isoindole-3,4′-piperidin]-7-yl)piperidine-2,6-dione hydrochloride (30.0 mg, yield 54%) as a gray solid. The crude product was directly used in next step without further purification. LC-MS (ESI): mass calcd. for C₂₀H₂₃N₃O₄, 369.42; m/z found, 370.1 [M+H]⁺.

### Intermediate B9: 3-(3′-hydroxy-6-oxo-6,8-dihydro-2H,7H-spiro[furo[2,3-e]isoindole-3,4′-piperidin]-7-yl)piperidine-2,6-dione

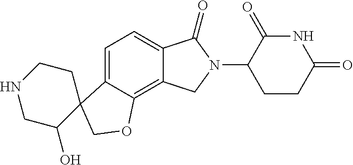 [SMILES: O=C1CCC(N2Cc3c(ccc4c3OCC43CCNCC3O)C2=O)C(=O)N1]

### Step A: 1′-(tert-butoxycarbonyl)-3′-hydroxy-7-(hydroxymethyl)-2H-spiro[benzofuran-3,4′-piperidine]-6-carboxylic acid

To a mixture of tert-butyl 3′-hydroxy-6-oxo-6,8-dihydro-2H-spiro[benzo[2,1-b:3,4-c′]difuran-3,4′-piperidine]-1′-carboxylate (400 mg, 1.11 mmol, 1.0 eq) in THF (9.00 mL), MeOH (9.00 mL), and H₂O (3.00 mL) was added NaOH (89 mg, 2.21 mmol, 2.0 eq) and the reaction mixture was stirred at 40° C. for 1 h. After cooled to room temperature, the reaction mixture was diluted with EA (20 mL), adjusted to pH=4-5 with aqueous HCl solution (3 N), and extracted with EA (40 mL×4). The organic layer was washed with brine (20 mL×2), dried over anhydrous sodium sulfate, filtered, and concentrated under reduced pressure to provide 1′-(tert-butoxycarbonyl)-3′-hydroxy-7-(hydroxymethyl)-2H-spiro[benzofuran-3,4′-piperidine]-6-carboxylic acid (400 mg, yield 95%) as a colorless oil. The crude product was directly used in next step without purification. LC-MS (ESI): mass calcd. for C₁₉H₂₅NO₇, 379.41; m/z found, 378.3 [M−H]⁻.

### Step B: 1′-(tert-butoxycarbonyl)-7-formyl-3′-hydroxy-2H-spiro[benzofuran-3,4′-piperidine]-6-carboxylic acid

A mixture of 1′-(tert-butoxycarbonyl)-3′-hydroxy-7-(hydroxymethyl)-2H-spiro[benzofuran-3,4′-piperidine]-6-carboxylic acid (420 mg, 1.11 mmol, 1.0 eq) and active manganese dioxide (1.92 g, 22.1 mmol, 20.0 eq) in DCM (40.0 mL) was stirred at room temperature for 16 h. After filtration via a short column, the filtrate was collected and concentrated under reduced pressure to 1′-(tert-butoxycarbonyl)-7-formyl-3′-hydroxy-2H-spiro[benzofuran-3,4′-piperidine]-6-carboxylic acid (260 mg, yield 62%) as a colorless oil. The crude product was directly used in next step without purification. LC-MS (ESI): mass calcd. for C₁₉H₂₁F₂NO₆, 377.39; m/z found, 378.1 [M+H]⁺.

### Step C: tert-butyl 7-(2,6-dioxopiperidin-3-yl)-3′-hydroxy-6-oxo-7,8-dihydro-2H,6H-spiro[furo[2,3-e]isoindole-3,4′-piperidine]-1′-carboxylate

To a solution of 1′-(tert-butoxycarbonyl)-7-formyl-3′-hydroxy-2H-spiro[benzofuran-3,4′-piperidine]-6-carboxylic acid (260 mg, 689 μmol, 1.0 eq) and 3-aminopiperidine-2,6-dione hydrochloride (227 mg, 1.38 mmol, 2.0 eq) in DMF (10.0 mL) was added Acetic acid (1.0 mL) and the reaction mixture was stirred at 40° C. for 2 h. Sodium triacetoxyborohydride (438 mg, 2.07 mmol, 3.0 eq) was added to above mixture and the resulting mixture was stirred at 40° C. for 16 h. After cooled to room temperature, the mixture was dissolve in EA (40 mL), washed with brine (30 mL×3), dried over anhydrous sodium sulfate, filtered and concentrated under reduced pressure. The residue was purified by flash column chromatography on silica gel (MeOH in DCM, 10%) to afford tert-butyl 7-(2,6-dioxopiperidin-3-yl)-3′-hydroxy-6-oxo-7,8-dihydro-2H,6H-spiro[furo[2,3-e]isoindole-3,4′-piperidine]-1′-carboxylate (260 mg, yield 80%) as a grey solid. LC-MS (ESI): mass calcd. for C₂₄H₂₉N₃O₇, 471.51; m/z found, 472.2 [M+H]⁺.

### Step D: 3-(3′-hydroxy-6-oxo-6,8-dihydro-2H,7H-spiro[furo[2,3-e]isoindole-3,4′-piperidin]-7-yl)piperidine-2,6-dione

To a solution of tert-butyl 7-(2,6-dioxopiperidin-3-yl)-3′-hydroxy-6-oxo-7,8-dihydro-2H,6H-spiro[furo[2,3-e]isoindole-3,4′-piperidine]-1′-carboxylate (260 mg, 551 μmol, 1.0 eq) in DCM (5.0 mL) was added TFA (1.7 mL, 22.1 mmol, 40.0 eq) and the reaction mixture was stirred at room temperature for 6 h. The mixture was cautiously concentrated under reduced pressure to afford 3-(3′-hydroxy-6-oxo-6,8-dihydro-2H,7H-spiro[furo[2,3-e]isoindole-3,4′-piperidin]-7-yl)piperidine-2,6-dione trifluoroacetate (100 mg, yield 48%) as a yellow oil. The crude product was directly used in next step without further purification. LC-MS (ESI): mass calcd. for C₁₉H₂₁N₃O₅, 371.39; m/z found, 372.2 [M+H]⁺.

### Intermediate B10. 3-(3′,3′-difluoro-6-oxo-6,8-dihydro-2H,7H-spiro[furo[2,3-e]isoindole-3,4′-piperidin]-7-yl)piperidine-2,6-dione

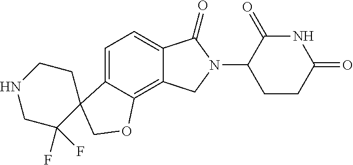 [SMILES: O=C1CCC(N2Cc3c(ccc4c3OCC43CCNCC3(F)F)C2=O)C(=O)N1]

### Step A: tert-butyl 4-(((5-bromo-1-oxo-1,3-dihydroisobenzofuran-4-yl)oxy)methyl)-3,6-dihydropyridine-1(2H)-carboxylate

To a solution of 5-bromo-4-hydroxyisobenzofuran-1(3H)-one (5.0 g, 21.80 mmol, 1.0 eq) in THF (150 mL) were added tert-butyl-4-(hydroxymethyl)-3,6-dihydropyridine-1(2H)-carboxylate (5.59 g, 26.2 mmol, 1.2 eq) and Triphenylphosphine (8.59 g, 32.7 mmol, 7.31 mL, 1.5 eq). The mixture was stirred under N₂ at 0° C. for 20 min. Then DIAD (6.62 g, 32.7 mmol, 6.37 mL, 1.5 eq) was dropwise added to above mixture and the mixture was stirred at room temperature overnight. After evaporation, the crude product was purified by flash column chromatography on silica gel (ethyl acetate in petroleum ether, from 0% to 20%) to afford tert-butyl 4-(((5-bromo-1-oxo-1,3-dihydroisobenzofuran-4-yl)oxy)methyl)-3,6-dihydropyridine-1(2H)-carboxylate (9.0 g, yield 97%) as a yellow solid. LC-MS (ESI): mass calcd. for C₁₉H₂₂BrNO₅, 424.29; m/z found, 369.9 [M+H−56]⁺. ¹H NMR (400 MHz, DMSO-d₆) δ 7.83 (d, J=8.0 Hz, 1H), 7.45 (d, J=8.0 Hz, 1H), 5.89 (s, 1H), 5.66 (s, 2H), 4.66 (s, 2H), 3.88 (s, 2H), 3.48 (dd, J=14.6, 9.0 Hz, 2H), 2.22 (s, 2H), 1.41 (s, 9H).

### Step B: tert-butyl 6-oxo-2′,3′,6,8-tetrahydro-1′H,2H-spiro[benzo[2,1-b:3,4-c′]difuran-3,4′-pyridine]-1′-carboxylate

To a solution of tert-butyl 4-(((5-bromo-1-oxo-1,3-dihydroisobenzofuran-4-yl)oxy)methyl)-3,6-dihydropyridine-1(2H)-carboxylate (5.00 g, 7.90 mmol, 1.0 eq) in DMF (30.0 mL) were added sodium formate (591 mg, 8.69 mmol, 1.1 eq), palladium diacetate (177 mg, 790 μmol, 0.1 eq), sodium acetate (1.62 g, 19.7 mmol, 2.5 eq), and TEA (1.44 g, 8.69 mmol, 1.33 mL, 1.1 eq). The mixture was stirred under N₂ at 70° C. for 16 h. After cooled to room temperature, the mixture was filtered and the cake was washed with EA (100 mL). The filtrate was washed with brine (60 mL×3), dried over anhydrous sodium sulfate, filtered and concentrated under reduced pressure. The residue was purified with flash column chromatography on silica gel (ethyl acetate in petroleum ether, from 0% to 30%) to afford tert-butyl 6-oxo-2′,3′,6,8-tetrahydro-1′H,2H-spiro[benzo[2,1-b:3,4-c′]difuran-3,4′-pyridine]-1′-carboxylate (1.70 g, yield 62.7%) as a colorless oil. LC-MS (ESI): mass calcd. for C₁₉H₂₁NO₅, 343.38; m/z found, 344.1 [M+H]⁺. ¹H NMR (400 MHz, DMSO-d₆) δ 7.41 (d, J=7.6 Hz, 1H), 7.34 (d, J=7.6 Hz, 1H), 7.03-6.98 (m, 1H), 5.38 (s, 2H), 4.88-4.82 (m, 1H), 4.60 (d, J=9.2 Hz, 1H), 4.39 (d, J=9.2 Hz, 1H), 3.81-3.75 (m, 1H), 3.47-3.41 (m, 1H), 2.02-1.98 (m, 1H), 1.84-1.78 (m, 1H), 1.47 (s, 9H).

### Step C: tert-butyl 3′-hydroxy-6-oxo-6,8-dihydro-2H-spiro[benzo[2,1-b:3,4-c′]difuran-3,4′-piperidine]-1′-carboxylate

To a solution of tert-butyl 6-oxo-2′,3′,6,8-tetrahydro-1′H,2H-spiro[benzo[2,1-b:3,4-c′]difuran-3,4′-pyridine]-1′-carboxylate (4.70 g, 13.7 mmol, 1.0 eq) in THF (60.0 mL) was added BH₃-THF (1 N) (34.2 mL, 34.2 mmol, 2.5 eq) under N₂ at −78° C. The reaction was allowed to slowly warm to 0° C. and stirred at 0° C. for 5 h. Water (5 mL) was added to above mixture, followed by Sodium perborate (5.60 g, 68.4 mmol, 5.0 eq). The resulting mixture was stirred overnight. The mixture was diluted with DCM (100 mL), washed with brine (50 mL×2), dried over anhydrous sodium sulfate, filtered and concentrated under reduced pressure. The residue was purified with flash column chromatography on silica gel (ethyl acetate in petroleum ether, from 0% to 40%) to afforded tert-butyl 3′-hydroxy-6-oxo-6,8-dihydro-2H-spiro[benzo[2,1-b:3,4-c′]difuran-3,4′-piperidine]-1′-carboxylate (2.70 g, yield 54%) as a white powder. LC-MS (ESI): mass calcd. for C₁₉H₂₃NO₆, 361.39; m/z found, 306.1 [M+H−56]⁺. ¹H NMR (400 MHz, DMSO-d₆) δ 7.51 (d, J=7.6 Hz, 1H), 7.37 (d, J=7.6 Hz, 1H), 5.35 (d, J=2.6 Hz, 2H), 5.31 (d, J=4.6 Hz, 1H), 4.83 (d, J=9.0 Hz, 1H), 4.53 (d, J=9.0 Hz, 1H), 4.01 (s, 1H), 3.87 (s, 1H), 3.76-3.73 (m, 1H), 2.83-2.56 (m, 2H), 1.88-1.74 (m, 2H), 1.43 (s, 9H).

### Step D: tert-butyl 3′,6-dioxo-6,8-dihydro-2H-spiro[benzo[2,1-b:3,4-c′]difuran-3,4′-piperidine]-1′-carboxylate

To a solution of tert-butyl 3′-hydroxy-6-oxo-6,8-dihydro-2H-spiro[benzo[2,1-b:3,4-c′]difuran-3,4′-piperidine]-1′-carboxylate (1.80 g, 4.98 mmol, 1.0 eq) in DCM (30.0 mL) was added Dess-Martin Periodinane (5.28 g, 12.5 mmol, 2.5 eq). The mixture was stirred at room temperature for 4 h. The reaction was diluted with DCM (60 mL), washed with aqueous sodium thiosulfate solution (30 mL×2) and washed with brine (40 mL×2). The organic layer was dried over anhydrous sodium sulfate, filtered, and concentrated under reduced pressure. The residue was purified by flash column chromatography on silica gel (ethyl acetate in petroleum ether, from 0% to 40%) to afford tert-butyl 3′,6-dioxo-6,8-dihydro-2H-spiro[benzo[2,1-b:3,4-c′]difuran-3,4′-piperidine]-1′-carboxylate (1.3 g, yield 72.6%) as a colorless oil. LC-MS (ESI): mass calcd. for C₁₉H₂₁NO₆, 359.38; m/z found, 360.1 [M+H]⁺.

### Step E: tert-butyl 3′,6-dioxo-6,8-dihydro-2H-spiro[benzo[2,1-b:3,4-c′]difuran-3,4′-piperidine]-1′-carboxylate

To a solution of tert-butyl 3′,6-dioxo-6,8-dihydro-2H-spiro[benzo[2,1-b:3,4-c′]difuran-3,4′-piperidine]-1-carboxylate (1.20 g, 3.34 mmol, 1.0 eq), triethylammonium fluoride (3.23 g, 3.27 mL, 20.0 mmol, 6.0 eq), and N,N-diethyl-S,S-difluoro-sulfiliminium tetrafluoroborate (3.44 g, 15.0 mmol, 4.5 eq) in DCM (50.0 mL) was added TEA (845 mg, 1.16 mL, 8.35 mmol, 2.5 eq) at 25° C. and the mixture was stirred at 25° C. for 1 h. The reaction mixture was quenched with saturated aqueous NaHCO₃ solution (30 mL) and extracted with DCM (30 mL×3). The separated organic phase was washed with brine (30 mL), dried over anhydrous Na₂SO₄, filtered, and concentrated under reduced pressure. The residue was purified by silica gel chromatography on silica gel (ethyl acetate in petroleum ether, from 0% to 30%) to afford tert-butyl 3′,3′-difluoro-6-oxo-6,8-dihydro-2H-spiro[benzo[2,1-b:3,4-c′]difuran-3,4′-piperidine]-1′-carboxylate (260 mg, yield 20%) as a yellow oil. LC-MS (ESI): mass calcd. for C₁₉H₂₁F₂NO₅, 381.38; m/z found, 382.1 [M+H]⁺.

### Step F: 1′-(tert-butoxycarbonyl)-3′,3′-difluoro-7-(hydroxymethyl)-2H-spiro[benzofuran-3,4′-piperidine]-6-carboxylic acid

To a solution of tert-butyl 3′,3′-difluoro-6-oxo-6,8-dihydro-2H-spiro[benzo[2,1-b:3,4-c′]difuran-3,4′-piperidine]-1′-carboxylate (280 mg, 734 μmol, 1.0 eq) in THF (9.00 mL), MeOH (9.00 mL), and H₂O (3.00 mL) was added NaOH (44 mg, 1.10 mmol, 1.5 eq). The mixture was stirred at 40° C. for 1 h. After cooling to room temperature, the reaction mixture was diluted with EA (20 mL), adjusted to pH=4-5 with aqueous HCl solution (3 N), and extracted with EA (40 mL×4). The organic layer was washed with brine (20 mL×2), dried over anhydrous sodium sulfate, filtered, and concentrated to 1′-(tert-butoxycarbonyl)-3′,3′-difluoro-7-(hydroxymethyl)-2H-spiro[benzofuran-3,4′-piperidine]-6-carboxylic acid (290 mg, yield 99%) as a colorless oil. The crude product was directly used in next step without purification. LC-MS (ESI): mass calcd. for C₁₉H₂₃F₂NO₆, 399.39; m/z found, 398.3 [M−H]⁻.

### Step G: 1′-(tert-butoxycarbonyl)-3′,3′-difluoro-7-formyl-2H-spiro[benzofuran-3,4′-piperidine]-6-carboxylic acid

A solution of 1′-(tert-butoxycarbonyl)-3′,3′-difluoro-7-(hydroxymethyl)-2H-spiro[benzofuran-3,4′-piperidine]-6-carboxylic acid (260 mg, 651 μmol, 1.0 eq) and active Manganese dioxide (1.13 g, 13.0 mmol, 20.0 eq) in DCM (20.0 mL) was stirred at room temperature for 16 h. After filtration via a short column, the filtrate is collected and concentrated under reduced pressure to 1′-(tert-butoxycarbonyl)-3′,3′-difluoro-7-formyl-2H-spiro[benzofuran-3,4′-piperidine]-6-carboxylic acid (250 mg, yield 96.6%) as a colorless oil. The crude product was directly used in next step without purification. LC-MS (ESI): mass calcd. for C₁₉H₂₁F₂NO₆, 397.37; m/z found, 398.1 [M+H]⁺.

### Step H: tert-butyl 7-(2,6-dioxopiperidin-3-yl)-3,3′-difluoro-6-oxo-7,8-dihydro-2H,6H-spiro[furo[2,3-e]isoindole-3,4′-piperidine]-1′-carboxylate

To a solution of 1′-(tert-butoxycarbonyl)-3′,3′-difluoro-7-formyl-2H-spiro[benzofuran-3,4′-piperidine]-6-carboxylic acid (260 mg, 654 μmol, 1.0 eq) and 3-aminopiperidine-2,6-dione hydrochloride (215 mg, 1.31 mmol, 2.0 eq) in DMF (10.0 mL) was added Acetic acid (0.52 g, 0.50 mL, 8.7 mmol, 13.0 eq) and the reaction was stirred at 40° C. for 2 h. Sodium triacetoxyborohydride (416 mg, 1.96 mmol, 3.0 eq) was added to above mixture and the resulting mixture was stirred at 40° C. for 16 h. After cooled to room temperature, the mixture was dissolve with EA (60 mL), washed with brine (30 mL×4), dried over anhydrous sodium sulfate, filtered, and concentrated under reduced pressure. The residue was purified with flash column chromatography on silica gel (ethyl acetate in petroleum ether, from 0% to 90%) to afford tert-butyl 7-(2,6-dioxopiperidin-3-yl)-3′,3′-difluoro-6-oxo-7,8-dihydro-2H,6H-spiro[furo[2,3-e]isoindole-3,4′-piperidine]-1′-carboxylate (220 mg, yield 68.4%) as a grey solid. LC-MS (ESI): mass calcd. for C₂₄H₂₇F₂N₃O₆, 491.49; m/z found, 492.2 [M+H]⁺.

### Step I: afford 3-(3′,3′-difluoro-6-oxo-6,8-dihydro-2H,7H-spiro[furo[2,3-e]isoindole-3,4′-piperidin]-7-yl)piperidine-2,6-dione

To a solution of tert-butyl 7-(2,6-dioxopiperidin-3-yl)-3′,3′-difluoro-6-oxo-7,8-dihydro-2H,6H-spiro[furo[2,3-e]isoindole-3,4′-piperidine]-1′-carboxylate (200 mg, 407 μmol, 1.0 eq) in DCM (5.00 mL) was added Trifluoroacetic acid (2.99 g, 2.00 mL, 26.3 mmol, 64.5 eq) and the reaction was stirred at 25° C. for 1 h. The mixture was concentrated and dried to afford 3-(3′,3′-difluoro-6-oxo-6,8-dihydro-2H,7H-spiro[furo[2,3-e]isoindole-3,4′-piperidin]-7-yl)piperidine-2,6-dione trifluoroacetate (150 mg, yield 94%) as a grey solid. LC-MS (ESI): mass calcd. for C₁₉H₁₉F₂N₃O₄, 391.37; m/z found, 392.1 [M+H]⁺. ¹H NMR (400 MHz, DMSO-d₆) δ 10.99 (d, J=4.4 Hz, 1H), 9.67 (s, 1H), 7.48-7.37 (m, 2H), 5.16-4.96 (m, 2H), 4.74-4.63 (m, 1H), 4.44 (t, J=17.0 Hz, 1H), 4.27 (t, J=16.6 Hz, 1H), 3.93-3.71 (m, 2H), 3.03 (t, J=12.0 Hz, 1H), 2.97-2.83 (m, 1H), 2.60 (d, J=17.2 Hz, 1H), 2.46-2.40 (m, 2H), 2.26-2.23 (m, 1H), 2.08 (d, J=5.0 Hz, 1H), 2.00-1.98 (m, 1H).

### Intermediate B11: (3S)-3-(3′,3′-difluoro-6-oxo-6,8-dihydro-2H,7H-spiro[furo[2,3-e]isoindole-3,4′-piperidin]-7-yl)piperidine-2,6-dione

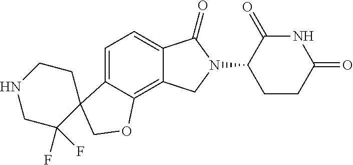 [SMILES: O=C1CC[C@H](N2Cc3c(ccc4c3OCC43CCNCC3(F)F)C2=O)C(=O)N1]

### Step A: tert-butyl 4-(((5-bromo-1-oxo-1,3-dihydroisobenzofuran-4-yl)oxy)methyl)-3,6-dihydropyridine-1(2H)-carboxylate

To a solution of 5-bromo-4-hydroxyisobenzofuran-1(3H)-one (10.0 g, 43.60 mmol, 1.0 eq) in THF (300 mL) were added tert-butyl-4-(hydroxymethyl)-3,6-dihydropyridine-1(2H)-carboxylate (11.18 g, 52.4 mmol, 1.2 eq) and Triphenylphosphine (17.18 g, 65.4 mmol, 14.62 mL, 1.5 eq). The mixture was stirred under N₂ at 0° C. for 20 min. Then DIAD (13.24 g, 65.4 mmol, 12.74 mL, 1.5 eq) was dropwise added to above mixture and the mixture was stirred at room temperature overnight. After evaporation, the crude product was purified by flash column chromatography on silica gel (ethyl acetate in petroleum ether, from 0% to 20%) to afford tert-butyl 4-(((5-bromo-1-oxo-1,3-dihydroisobenzofuran-4-yl)oxy)methyl)-3,6-dihydropyridine-1(2H)-carboxylate (18.0 g, yield 97%) as a yellow solid. LC-MS (ESI): mass calcd. for C₁₉H₂₂BrNO₅, 424.29; m/z found, 369.9 [M+H-56]⁺. ¹H NMR (400 MHz, DMSO-d₆) δ 7.83 (d, J=8.0 Hz, 1H), 7.45 (d, J=8.0 Hz, 1H), 5.89 (s, 1H), 5.66 (s, 2H), 4.66 (s, 2H), 3.88 (s, 2H), 3.48 (dd, J=14.6, 9.0 Hz, 2H), 2.22 (s, 2H), 1.41 (s, 9H).

### Step B: tert-butyl 6-oxo-2′,3′,6,8-tetrahydro-1′H,2H-spiro[benzo[2,1-b:3,4-c′]difuran-3,4′-pyridine]-1′-carboxylate

To a solution of tert-butyl 4-(((5-bromo-1-oxo-1,3-dihydroisobenzofuran-4-yl)oxy)methyl)-3,6-dihydropyridine-1(2H)-carboxylate (15.00 g, 23.70 mmol, 1.0 eq) in DMF (90.0 mL) were added sodium formate (1.77 g, 26.07 mmol, 1.1 eq), palladium diacetate (531 mg, 2.37 mmol, 0.1 eq), sodium acetate (4.86 g, 59.1 mmol, 2.5 eq), and TEA (4.32 g, 26.07 mmol, 3.99 mL, 1.1 eq). The mixture was stirred under N₂ at 70° C. for 16 h. After cooled to room temperature, the mixture was filtered and the cake was washed with EA (300 mL). The filtrate was washed with brine (60 mL×3), dried over anhydrous sodium sulfate, filtered and concentrated under reduced pressure. The residue was purified with flash column chromatography on silica gel (ethyl acetate in petroleum ether, from 0% to 30%) to afford tert-butyl 6-oxo-2′,3′,6,8-tetrahydro-1′H,2H-spiro[benzo[2,1-b:3,4-c′]difuran-3,4′-pyridine]-1′-carboxylate (5.10 g, yield 62.7%) as a colorless oil. LC-MS (ESI): mass calcd. for C₁₉H₂₁NO₅, 343.38; m/z found, 344.1 [M+H]⁺. ¹H NMR (400 MHz, DMSO-d₆) δ 7.41 (d, J=7.6 Hz, 1H), 7.34 (d, J=7.6 Hz, 1H), 7.03-6.98 (m, 1H), 5.38 (s, 2H), 4.88-4.82 (m, 1H), 4.60 (d, J=9.2 Hz, 1H), 4.39 (d, J=9.2 Hz, 1H), 3.81-3.75 (m, 1H), 3.47-3.41 (m, 1H), 2.02-1.98 (m, 1H), 1.84-1.78 (m, 1H), 1.47 (s, 9H).

### Step C: tert-butyl 3′-hydroxy-6-oxo-6,8-dihydro-2H-spiro[benzo[2,1-b:3,4-c′]difuran-3,4′-piperidine]-1′-carboxylate

To a solution of tert-butyl 6-oxo-2′,3′,6,8-tetrahydro-1′H,2H-spiro[benzo[2,1-b:3,4-c′]difuran-3,4′-pyridine]-1′-carboxylate (5.10 g, 14.87 mmol, 1.0 eq) in THF (60.0 mL) was added BH₃-THF (1 N) (37.1 mL, 37.1 mmol, 2.5 eq) under N₂ at −78° C. The reaction was allowed to slowly warm to 0° C. and stirred at 0° C. for 5 h. Water (10 mL) was added to above mixture, followed by Sodium perborate (6.07 g, 74.2 mmol, 5.0 eq). The resulting mixture was stirred overnight. The mixture was diluted with DCM (100 mL), washed with brine (50 mL×2), dried over anhydrous sodium sulfate, filtered and concentrated under reduced pressure. The residue was purified with flash column chromatography on silica gel (ethyl acetate in petroleum ether, from 0% to 40%) to afforded tert-butyl 3′-hydroxy-6-oxo-6,8-dihydro-2H-spiro[benzo[2,1-b:3,4-c′]difuran-3,4′-piperidine]-1′-carboxylate (2.93 g, yield 54%) as a white powder. LC-MS (ESI): mass calcd. for C₁₉H₂₃NO₆, 361.39; m/z found, 306.1 [M+H−56]⁺. ¹H NMR (400 MHz, DMSO-d₆) δ 7.51 (d, J=7.6 Hz, 1H), 7.37 (d, J=7.6 Hz, 1H), 5.35 (d, J=2.6 Hz, 2H), 5.31 (d, J=4.6 Hz, 1H), 4.83 (d, J=9.0 Hz, 1H), 4.53 (d, J=9.0 Hz, 1H), 4.01 (s, 1H), 3.87 (s, 1H), 3.76-3.73 (m, 1H), 2.83-2.56 (m, 2H), 1.88-1.74 (m, 2H), 1.43 (s, 9H).

### Step D: tert-butyl 3′,6-dioxo-6,8-dihydro-2H-spiro[benzo[2,1-b:3,4-c′]difuran-3,4′-piperidine]-1′-carboxylate

To a solution of tert-butyl 3′-hydroxy-6-oxo-6,8-dihydro-2H-spiro[benzo[2,1-b:3,4-c′]difuran-3,4′-piperidine]-1′-carboxylate (2.90 g, 8.10 mmol, 1.0 eq) in DCM (60.0 mL) was added Dess-Martin Periodinane (8.60 g, 20.35 mmol, 2.5 eq). The mixture was stirred at room temperature for 4 h. The reaction mixture was quenched with aqueous sodium thiosulfate solution (60 mL) and extracted with DCM (90 mL×3). The organic layer was washed with saturated aqueous NaHCO₃ solution (60 mL×2), brine (40 mL×2), dried over anhydrous sodium sulfate, and filtered. The filtrate was concentrated under reduced pressure to afford tert-butyl 3′,6-dioxo-6,8-dihydro-2H-spiro[benzo[2,1-b:3,4-c′]difuran-3,4′-piperidine]-1′-carboxylate (2.12 g, yield 72.6%) as a colorless oil. The crude product was directly used in next step without further purification. LC-MS (ESI): mass calcd. for C₁₉H₂₁NO₆, 359.38; m/z found, 360.1 [M+H]⁺.

### Step E: tert-butyl 3′,6-dioxo-6,8-dihydro-2H-spiro[benzo[2,1-b:3,4-c′]difuran-3,4′-piperidine]-1′-carboxylate

To a solution of tert-butyl 3′,6-dioxo-6,8-dihydro-2H-spiro[benzo[2,1-b:3,4-c′]difuran-3,4′-piperidine]-1′-carboxylate (2.0 g, 5.57 mmol, 1.0 eq), triethylammoniumFluoride (5.38 g, 5.44 mL, 33.4 mmol, 6.0 eq), and N,N-Diethyl-S,S-difluoro-sulfiliminium tetrafluoroborate (XtalFluor-E) (5.73 g, 25.0 mmol, 4.5 eq) in DCM (100.0 mL) was added TEA (1.41 g, 1.94 mL, 13.9 mmol, 2.5 eq) at 0° C. and the mixture was stirred at 25° C. for 1 h. The reaction mixture was quenched with saturated aqueous NaHCO₃ solution (60 mL) and extracted with DCM (100 mL×3). The separated organic phase was washed with brine (100 mL×3), dried over anhydrous Na₂SO₄, filtered, and concentrated under reduced pressure. The residue was purified by silica gel chromatography on silica gel (ethyl acetate in petroleum ether, from 0% to 30%) to afford tert-butyl 3′,3′-difluoro-6-oxo-6,8-dihydro-2H-spiro[benzo[2,1-b:3,4-c′]difuran-3,4′-piperidine]-1′-carboxylate (1.0 g, yield 47%) as a yellow oil. LC-MS (ESI): mass calcd. for C₁₉H₂₁F₂NO₅, 381.38; m/z found, 382.1 [M+H]⁺.

### Step F: 1′-(tert-butoxycarbonyl)-3′,3′-difluoro-7-(hydroxymethyl)-2H-spiro[benzofuran-3,4′-piperidine]-6-carboxylic acid

To a solution of tert-butyl 3′,3′-difluoro-6-oxo-6,8-dihydro-2H-spiro[benzo[2,1-b:3,4-c′]difuran-3,4′-piperidine]-1′-carboxylate (280 mg, 734 μmol, 1.0 eq) in THF (9.00 mL), MeOH (9.00 mL), and H₂O (3.00 mL) was added NaOH (44 mg, 1.10 mmol, 1.5 eq). The mixture was stirred at 40° C. for 1 h. After cooling to room temperature, the reaction mixture was diluted with EA (20 mL), adjusted to pH=4-5 with aqueous HCl solution (3 N), and extracted with EA (40 mL×4). The organic layer was washed with brine (20 mL×2), dried over anhydrous sodium sulfate, filtered, and concentrated to 1′-(tert-butoxycarbonyl)-3′,3′-difluoro-7-(hydroxymethyl)-2H-spiro[benzofuran-3,4′-piperidine]-6-carboxylic acid (290 mg, yield 99%) as a colorless oil. The crude product was directly used in next step without purification. LC-MS (ESI): mass calcd. for C₁₉H₂₃F₂NO₆, 399.39; m/z found, 398.3 [M−H]⁻.

### Step G: tert-butyl 2,2-difluoro-8-hydroxy-6-oxo-6,8-dihydro-2H-spiro[benzo[2,1-b:3,4-c′]difuran-3,4′-piperidine]-1′-carboxylate

A solution of 1′-(tert-butoxycarbonyl)-3′,3′-difluoro-7-(hydroxymethyl)-2H-spiro[benzofuran-3,4′-piperidine]-6-carboxylic acid (1.6 g, 4.01 mmol, 1.0 eq) and active Manganese dioxide (6.97 g, 80.1 mmol, 20.0 eq) in DCM (30.0 mL) was stirred at room temperature for 16 h. After filtration via a short column, the filtrate is collected and concentrated under reduced pressure to tert-butyl 2,2-difluoro-8-hydroxy-6-oxo-6,8-dihydro-2H-spiro[benzo[2,1-b:3,4-c′]difuran-3,4′-piperidine]-1′-carboxylate (1.5 g, yield 96.6%) as a colorless oil. The crude product was directly used in next step without purification. LC-MS (ESI): mass calcd. for C₁₉H₂₁F₂NO₆, 397.37; m/z found, 398.1 [M+H]⁺.

### Step H: tert-butyl 7-((S)-1-amino-5-(tert-butoxy)-1,5-dioxopentan-2-yl)-3′,3′-difluoro-6-oxo-7,8-dihydro-2H,6H-spiro[furo[2,3-e]isoindole-3,4′-piperidine]-1′-carboxylate

To a solution of tert-butyl 3′,3′-difluoro-8-hydroxy-6-oxo-6,8-dihydro-2H-spiro[benzo[2,1-b:3,4-c′]difuran-3,4′-piperidine]-1′-carboxylate (1.60 g, 4.03 mmol, 1.0 eq) and tert-butyl (S)-4,5-diamino-5-oxopentanoate hydrochloride (1.92 g, 8.05 mmol, 2.0 eq) in DMF (30.0 mL) was added Acetic acid (2.42 g, 2.32 mL, 40.3 mmol, 10.0 eq) and the reaction mixture was stirred at 40° C. for 2 h. Sodium triacetoxyborohydride (2.56 g, 1.79 mL, 12.1 mmol, 3.0 eq) was added to above mixture and the resulting mixture was stirred at 40° C. for 16 h. After cooled to room temperature, the mixture was diluted with EA (100 mL), washed with brine (100 mL×3), dried over anhydrous sodium sulfate, filtered and concentrated under reduced pressure. The residue was purified with flash column chromatography on silica gel (ethyl acetate in petroleum ether, from 0% to 90%) to afford tert-butyl 7-((S)-1-amino-5-(tert-butoxy)-1,5-dioxopentan-2-yl)-3′,3′-difluoro-6-oxo-7,8-dihydro-2H,6H-spiro[furo[2,3-e]isoindole-3,4′-piperidine]-1′-carboxylate (1.80 g, yield 79%) as a white solid. LC-MS (ESI): mass calcd. for C₂₈H₃₇F₂N₃O₇, 565.61; m/z found, 566.4 [M+H]⁺.

### Step I: (3S)-3-(3′,3′-difluoro-6-oxo-6,8-dihydro-2H,7H-spiro[furo[2,3-e]isoindole-3,4′-piperidin]-7-yl)piperidine-2,6-dione benzenesulfonate

To a solution of tert-butyl 7-((S)-1-amino-5-(tert-butoxy)-1,5-dioxopentan-2-yl)-3′,3′-difluoro-6-oxo-7,8-dihydro-2H,6H-spiro[furo[2,3-e]isoindole-3,4′-piperidine]-1′-carboxylate (1.80 g, 3.18 mmol, 1.0 eq) in MeCN (30.0 mL) was added anhydrous benzenesulfonicacid (1.51 g, 9.55 mmol, 3.0 eq) and the reaction mixture was stirred under N₂ at 90° C. for 7 h. After cooled to room temperature, the mixture was concentrated and the residue was slurred with acetonitrile (30 mL) to afford (3S)-3-(3′,3′-difluoro-6-oxo-6,8-dihydro-2H,7H-spiro[furo[2,3-e]isoindole-3,4′-piperidin]-7-yl)piperidine-2,6-dione benzenesulfonate (1.20 g, yield 68%) as a white solid. LC-MS (ESI): mass calcd. for C₁₉H₁₉F₂N₃O₄, 391.37; m/z found, 392.1 [M+H]⁺.

¹H NMR (400 MHz, DMSO-d₆) δ 10.99 (d, J=4.6 Hz, 1H), 9.48 (s, 2H), 7.64-7.59 (m, 2H), 7.46 (d, J=7.6 Hz, 1H), 7.39 (d, J=7.7 Hz, 1H), 7.35-7.29 (m, 3H), 5.17-4.99 (m, 2H), 4.74-4.66 (m, 1H), 4.43 (t, J=17.2 Hz, 1H), 4.27 (t, J=17.2 Hz, 1H), 3.98-3.72 (m, 2H), 3.40 (d, J=13.0 Hz, 1H), 3.06-2.88 (m, 2H), 2.60-2.58 (m, 1H), 2.46-2.38 (m, 2H), 2.27-2.22 (m, 1H), 2.01-1.98 (m, 1H).

### Intermediate B12: 1-(4-chlorophenyl)-5-(trifluoromethyl)-1H-pyrazole-4-carbaldehyde

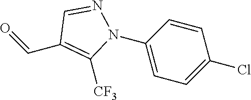 [SMILES: O=Cc1cnn(-c2ccc(Cl)cc2)c1C(F)(F)F]

### Step A: ethyl (Z)-2-(ethoxymethylene)-4,4,4-trifluoro-3-oxobutanoate

A mixture of ethyl 4,4,4-trifluoro-3-oxobutanoate (1.00 g, 5.43 mmol, 1.0 eq) and Acetic anhydride (1.66 g, 1.54 mL, 16.3 mmol, 3.0 eq) in Triethyl orthoformate (8.00 mL) was stirred at 130° C. for 4 h. The reaction mixture was concentrated under reduced pressure to give ethyl (Z)-2-(ethoxymethylene)-4,4,4-trifluoro-3-oxobutanoate (1.20 g, yield 92%) as a yellow oil. The crude product was directly used in next step without further purification. LC-MS: no ion signal was showed under routine conditions. LC-MS (ESI): mass calcd. for C₉H₁₁F₃O₄, 240.06; m/z found, 241.1 [M+H]⁺.

### Step B: ethyl 1-(4-chlorophenyl)-5-(trifluoromethyl)-1H-pyrazole-4-carboxylate

To a solution of ethyl (Z)-2-(ethoxymethylene)-4,4,4-trifluoro-3-oxobutanoate (1.20 g, 5.00 mmol, 1.0 eq) in EtOH (30.0 mL) were added Triethylamine (607 mg, 836 μL, 6.00 mmol, 1.2 eq) and (4-chlorophenyl) hydrazine hydrochloride (1.07 g, 6.00 mmol, 1.2 eq) at room temperature. The mixture was heated to 100° C. for 5 h. After evaporation, the residue was diluted with ethyl acetate (50 mL), washed with brine (30 mL×3), dried over anhydrous Na₂SO₄, filtered and concentrated under reduced pressure. The crude product was purified by flash column chromatography on silica gel (PE/EA=50/1) to provide ethyl 1-(4-chlorophenyl)-5-(trifluoromethyl)-1H-pyrazole-4-carboxylate (723 mg, yield 45%) as a yellow solid. LC-MS (ESI): mass calcd. for C₁₁H₈D₂F₂N₂O, 318.04; m/z found, 319.1 [M+H]⁺. ¹H NMR (400 MHz, DMSO-d₆) δ 8.32 (s, 1H), 7.69-7.66 (m, 2H), 7.63-7.60 (m, 2H), 4.32 (q, J=7.2 Hz, 2H), 1.31 (t, J=7.2 Hz, 3H).

### Step C: (1-(4-chlorophenyl)-5-(trifluoromethyl)-1H-pyrazol-4-yl)methanol

To a solution of ethyl 1-(4-chlorophenyl)-5-(trifluoromethyl)-1H-pyrazole-4-carboxylate (350 mg, 1.10 mmol, 1.0 eq) in THF (10.0 mL) was added Aluminum lithium hydride (83.4 mg, 2.20 mmol, 20 eq) at 0° C. The reaction mixture was stirring at 0° C. for 20 min. The reaction mixture was cautiously quenched by Na₂SO₄·10H₂O and stirred for 30 min. After filtration, the filtrate was diluted with water (10 mL) and extracted with ethyl acetate (20 mL×3). The combined organic extracts were washed with water (20 mL) and brine (20 mL), dried over anhydrous sodium sulfate, and filtered. The filtrate was concentrated under reduced pressure to give (1-(4-chlorophenyl)-5-(trifluoromethyl)-1H-pyrazol-4-yl)methanol (300 mg, yield 98%) as a yellow oil. The crude product was directly used in next step without further purification. LC-MS (ESI): mass calcd. for C₁₁H₈ClF₃N₂O, 276.03; m/z found, 277.1 [M+H]⁺.

### Step D: 1-(4-chlorophenyl)-5-(trifluoromethyl)-1H-pyrazole-4-carbaldehyde

To a solution of (1-(4-chlorophenyl)-5-(trifluoromethyl)-1H-pyrazol-4-yl)methanol (300 mg, 1.08 mmol, 1.0 eq) in DMSO (10.0 mL) was added IBX (759 mg, 2.71 mmol, 2.5 eq) at room temperature. The reaction mixture was stirred at 25° C. for 30 min. The reaction mixture was diluted with water (20 mL) and extracted with ethyl acetate (20 mL×3). The combined organic extracts were washed with saturated aqueous Na₂S₂O₃ solution (30 mL), saturated aqueous NaHCO₃ solution (30 mL) and brine (30 mL), dried over anhydrous sodium sulfate, filtered and concentrated under reduced pressure. The crude product was purified by flash column chromatography on silica gel (PE/EA=20/1) to provide 1-(4-chlorophenyl)-5-(trifluoromethyl)-1H-pyrazole-4-carbaldehyde (227 mg, yield 76%) as a yellow solid. LC-MS (ESI): mass calcd. for C₁₁H₆ClF₃N₂O, 274.01; m/z found, 275.1 [M+H]⁺. ¹H NMR (400 MHz, DMSO-d₆) δ 10.07 (s, 1H), 8.44 (s, 1H), 7.70 (d, J=8.8 Hz, 2H), 7.64 (d, J=8.8 Hz, 2H).

### Intermediate B13: 3-(4-chlorophenyl)isoxazole-5-carbaldehyde

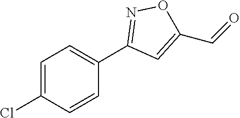 [SMILES: O=Cc1cc(-c2ccc(Cl)cc2)no1]

### Step A: (Z)-4-chlorobenzaldehyde oxime

A mixture of 4-chlorobenzaldehyde (6.00 g, 42.7 mmol, 1.0 eq), Hydroxylammoniumchloride (2.97 g, 42.7 mmol, 1.0 eq), and Cs₂CO₃ (2.26 g, 21.3 mmol, 0.5 eq) in MeOH (36.0 mL) and H₂O (18.00 mL) was stirred at 30° C. for 3 h. The reaction mixture was quenched with water (30 mL) and extracted with EtOAc (100 mL×3). The organic layer was dried over anhydrous Na₂SO₄, filtered and concentrated under reduced pressure. The residue was purified by flash column chromatography on silica gel (ethyl acetate in petroleum ether, from 0 to 20%) to afford (Z)-4-chlorobenzaldehyde oxime (5 g, yield 75%) as a white solid. LC-MS (ESI): mass calced for: C₇H₆ClNO 155.01; m/z found, 156.0 [M+H]⁺

### Step B: (E)-4-chloro-N-hydroxybenzimidoyl chloride

To a solution of (Z)-4-chlorobenzaldehyde oxime (1.00 g, 6.43 mmol, 1 eq) in DMF (20 mL) was added NCS (171.6 mg, 1.286 mmol, 0.2 eq). Then NCS (686.4 mg, 5.144 mmol, 0.8 eq) was added in small portions and the resulting mixture was stirred at 30° C. for 24 h. The reaction mixture was quenched with water (100 mL) and extracted with EtOAc (50 mL×3). The organic layer was washed with brine (50 mL×4), dried over anhydrous Na₂SO₄, filtered and concentrated under reduced pressure. The residue was purified by flash column chromatography on silica gel (ethyl acetate in petroleum ether, from 0 to 20%) to afford (E)-4-chloro-N-hydroxybenzimidoyl chloride (0.8 g, yield 66%) as a white solid. LC-MS (ESI): mass calced for: C₇H₅Cl₂NO 188.97; m/z found, 190.0 [M+H]⁺.

### Step C: (3-(4-chlorophenyl)isoxazol-5-yl)methanol

To a solution of (E)-4-chloro-N-hydroxybenzimidoyl chloride (0.6 g, 3.2 mmol, 1.0 eq) and Propargyl alcohol (0.19 g, 3.5 mmol, 1.1 eq) in t-BuOH (5.0 mL) and H₂O (5.0 mL) were added sodium ascorbate (63.0 mg, 0.32 mmol, 0.1 eq) and copper(II) sulfate pentahydrate (24 mg, 95.0 μmol, 0.03 e). The reaction mixture was stirred for 30 min. Then KHCO₃ (961 mg, 9.6 mmol, 3.0 eq) was added to above mixture and the resulting mixture was stirred at 30° C. for 1.5 h. The reaction mixture was quenched with water (30 mL) and extracted with EtOAc (30 mL×3). The organic layer was washed with brine (30 mL), dried over anhydrous Na₂SO₄, filtered and concentrated under reduced pressure. The residue was purified by flash column chromatography on silica gel (ethyl acetate in petroleum ether, from 0 to 35%) to afford (3-(4-chlorophenyl)isoxazol-5-yl)methanol (0.38 g, yield 57%) as a white solid. LC-MS (ESI): mass calced for: C₁₀H₈ClNO₂ 209.02; m/z found, 210.0 [M+H]⁺. ¹H NMR (400 MHz, DMSO-d₆) δ 7.88 (d, J=8.6 Hz, 1H), 7.58 (d, J=8.6 Hz, 1H), 6.96 (s, 1H), 5.73 (t, J=6.0 Hz, 1H), 4.61 (d, J=6.0 Hz, 2H).

### Step D: 3-(4-chlorophenyl)isoxazole-5-carbaldehyde

To a solution of (3-(4-chlorophenyl)isoxazol-5-yl)methanol (120 mg, 572 μmol, 1.0 eq) in DCM (5.0 mL) was added Dess-Martin Periodinane (267 mg, 630 μmol, 1.1 eq). The mixture was stirred at room temperature for 2 h. The reaction was diluted with EA (60 mL), washed with saturated aqueous NaS₂O₃ solution (30 mL), saturated aqueous NaHCO₃ solution (30 mL) and brine (30 mL), dried over anhydrous Na₂SO₄, filtered and concentrated under reduced pressure. The residue was purified by Prep-TLC (EA/PE=1/10) to give 3-(4-chlorophenyl)isoxazole-5-carbaldehyde (100 mg, yield 84%) as a yellow solid. LC-MS (ESI): mass calced for: C₁₀H₆ClNO₂ 207.01; m/z found, No MS signal found.

### Intermediate B14: 3-(4-chlorophenyl)isothiazole-5-carbaldehyde

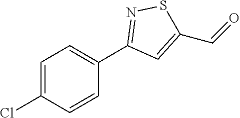 [SMILES: O=Cc1cc(-c2ccc(Cl)cc2)ns1]

### Step A: 3-(4-chlorophenyl)isothiazole

A mixture of 3-bromoisothiazole (100 mg, 610 μmol, 1.0 eq), (4-chlorophenyl) boronic acid (143 mg, 915 μmol, 1.5 eq), tetrakis(triphenylphosphine)palladium(o) (70.5 mg, 61.0 μmol, 0.1 eq), and Potassium carbonate (169 mg, 1.2 mmol, 2.0 eq) in 1,4-Dioxane (6.0 mL) and H₂O (1.0 mL) was stirred under N₂ at 80° C. for 4 h. After cooled to room temperature, the mixture was diluted with DCM (50 mL) and filtered. The filtrate was concentrated under reduced pressure and the residue was purified by Prep-TLC (EA/PE=1/10) to give 3-(4-chlorophenyl)isothiazole (82.0 mg, yield 68%) as a yellow oil. LC-MS (ESI): mass calcd. for C₉H₆ClNS, 195.0; m/z found, 196.1 (M+H)⁺. ¹H NMR (400 MHz, DMSO-d₆) δ 9.18 (d, J=0.6 Hz, 1H), 8.07 (d, J=8.4 Hz, 2H), 7.98 (d, J=0.6 Hz, 1H), 7.56 (d, J=8.4 Hz, 2H).

### Step B: 3-(4-chlorophenyl)isothiazole-5-carbaldehyde

To a mixture of 3-(4-chlorophenyl)isothiazole (50.0 mg, 256 μmol, 1.0 eq) in anhydrous THF (5.0 mL) was added n-butyllithium (2.5 Min n-hexane) (0.16 mL, 384 μmol, 1.5 eq) under N₂ at −78° C. and the mixture was stirred at this temperature for 30 min. Then anhydrous DMF (93.4 mg, 1.3 mmol, 5.0 eq) was added to above mixture and the resulting mixture was stirred under N₂ at −78° C. for 30 min. The mixture was quenched by the addition of saturated aqueous NH₄Cl solution (20 mL), stirred for 15 min, and extracted with EA (20 mL×4). The organic layer was washed with brine (20 mL), dried over anhydrous sodium sulfate, filtered and concentrated under reduced pressure. The residue was purified by Prep-TLC (EA/PE=1/10) to give 3-(4-chlorophenyl)isothiazole-5-carbaldehyde (28.0 mg, yield 49%) as a yellow solid. LC-MS (ESI): mass calcd. for C₁₀H₆ClNOS, 223.0; m/z found, 224.1 (M+H)+. ¹H NMR (400 MHz, DMSO-d₆) δ 10.19 (s, 1H), 8.72 (s, 1H), 8.11 (d, J=8.8 Hz, 2H), 7.62 (d, J=8.8 Hz, 2H).

### Intermediate B15: 3-chloro-1-(4-chlorophenyl)-1H-pyrazole-4-carbaldehyde

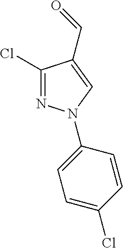 [SMILES: O=Cc1cn(-c2ccc(Cl)cc2)nc1Cl]

POCl₃ (5.51 g, 3.35 mL, 36.0 mmol, 7.0 eq) was added dropwise to DMF (10.0 mL) at 0° C. and the reaction mixture was stirred at 0° C. for 5 min. 1-(4-chlorophenyl)-1,2-dihydro-3H-pyrazol-3-one (1.00 g, 5.14 mmol, 1.0 eq) was added to above mixture and the resulting reaction mixture was heated to 105° C. and stirred for 16 h. After cooled to room temperature, the mixture was poured into crushed ice and brown solid was precipitated. The solid was collected by filtration, washed with water (30 mL). The solid cake was dissolved in DCM (150 mL), washed with brine (50 mL), dried over anhydrous Na₂SO₄, filtered and concentrated under reduced pressure. The residue was purified by flash column chromatography on silica gel (DCM/MeOH=10/1) to give 3-chloro-1-(4-chlorophenyl)-1H-pyrazole-4-carbaldehyde (40.0 mg, yield 3%) as a yellow solid. LC-MS (ESI): mass calced for: C₁₀H₈N₂O 172.19; m/z no mass signal. ¹H NMR (400 MHz, DMSO-d₆) δ 9.87 (s, 1H), 9.33 (s, 1H), 7.92 (d, J=8.8 Hz, 2H), 7.64 (d, J=8.8 Hz, 2H).

### Intermediate B16: 1-(4-chlorophenyl)-3-(trifluoromethyl)-1H-pyrazole-4-carbaldehyde

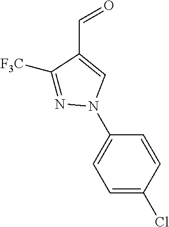 [SMILES: O=Cc1cn(-c2ccc(Cl)cc2)nc1C(F)(F)F]

A mixture of 3-(trifluoromethyl)-1H-pyrazole-4-carbaldehyde (50.0 mg, 305 μmol, 1.0 eq), Copper diacetate (111 mg, 609 μmol, 2.0 eq), (4-chlorophenyl)boronic acid (71.5 mg, 457 μmol, 1.5 eq), 2,2′-Bipyridine (95.2 mg, 609 μmol, 2.0 eq), and Sodium carbonate (161 mg, 1.5 mmol, 5.0 eq) in DCE (6.0 mL) was stirred under O₂ at 70° C. for 5 h. After cooled to room temperature, the mixture was filtered and the filtrate was quenched with water (20 m), and extracted with DCM (20 mL×3). The organic layer was washed with brine (20 mL), dried over anhydrous sodium sulfate, filtered and concentrated under reduced pressure. The crude product was purified by Prep-TLC (EA/PE=3/1) to give 1-(4-chlorophenyl)-3-(trifluoromethyl)-1H-pyrazole-4-carbaldehyde (34.0 mg, yield 41%) as a white solid. LC-MS (ESI): mass calcd. for C₁₁H₆ClF₃N₂O, 274.0; m/z no signal. ¹H NMR (400 MHz, DMSO-d₆) δ 9.98 (s, 1H), 9.51 (s, 1H), 7.98 (d, J=8.8 Hz, 2H), 7.68 (d, J=8.8 Hz, 2H).

### Intermediate B17: 1-((tetrahydro-2H-pyran-4-yl)methyl)-1H-pyrazole-4-carbaldehyde

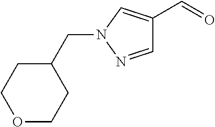 [SMILES: O=Cc1cnn(CC2CCOCC2)c1]

To a solution of 1H-pyrazole-4-carbaldehyde (1.95 g, 20.3 mmol, 1.0 eq) and Cs₂CO₃ (13.2 g, 40.5 mmol, 2.0 eq) in DMF (5.00 mL) was added 4-(bromomethyl)tetrahydro-2H-pyran (5.44 g, 30.4 mmol, 1.5 eq). The reaction mixture was stirred at 80° C. for 16 h. After cooled to room temperature, the mixture was diluted with EA (100 mL), washed with brine (50 mL×4), dried over anhydrous sodium sulfate, filtered and concentrated under reduced pressure. The residue was purified with flash column chromatography on silica gel (PE/EA=1/1) to obtain 1-((tetrahydro-2H-pyran-4-yl)methyl)-1H-pyrazole-4-carbaldehyde (2 g, yield 50%) as a yellow solid. LC-MS (ESI): mass calced for: C₁₀H₁₄N₂O₂ 194.11; m/z found, 195.1 [M+H]⁺.

### Intermediate B18: 6-(bromomethyl-d₂)-2-methyl-2H-indazole

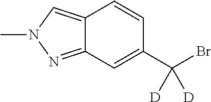

### Step A: (2-methyl-2H-indazol-6-yl)methan-d₂-ol

To a solution of methyl 2-methyl-2H-indazole-6-carboxylate (400 mg, 2.10 mmol, 1.0 eq) in THF (8.00 mL) was added LiAlD₄ (88.3 mg, 2.10 mmol, 1.0 eq) at 0° C. in portions and the reaction mixture was stirred at 0° C. for 0.5 h. The reaction mixture was diluted with EtOAc (30 mL), cautiously quenched with sodium sulfate decahydrate and stirred for 0.5 h. After filtration, the filtrate was concentrated under reduced pressure to afford (2-methyl-2H-indazol-6-yl)methan-d₂-ol (340 mg, yield 98%) as a yellow oil. LC-MS (ESI): mass calcd. for C₉H₈D₂N₂O, 164.20; m/z found, 165.1 [M+H]⁺.

### Step B: 6-(bromomethyl-d₂)-2-methyl-2H-indazole

To a solution of (2-methyl-2H-indazol-6-yl)methan-d₂-ol (340 mg, 2.07 mmol, 1.0 eq) in DCM (5.0 mL) was added Triphenylphosphine (815 mg, 3.11 mmol, 1.5 eq) and the mixture was stirred under the atmosphere at 50° C. for 5 min. Then a solution of CBr4 (1.03 g, 3.11 mmol, 1.5 eq) in DCM (5.0 mL) was added to above mixture and the reaction mixture was stirred at 50° C. for 2 h. After evaporation, the residue was purified by flash column chromatography on silica gel (PE/EA=2/1) to obtain 6-(bromomethyl-d₂)-2-methyl-2H-indazole (100 mg, yield 21%) as a yellow solid. LC-MS (ESI): mass calcd. for C₉H₇D₂BrN₂, 226.01; m/z found, 227.0 [M+H]⁺.

### Intermediate B19: 6-(bromomethyl-d₂)-1-methyl-1H-indazole

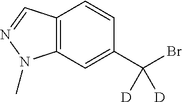

### Step A: methyl 1-methyl-1H-indazole-6-carboxylate and methyl 2-methyl-2H-indazole-6-carboxylate

To a solution of methyl 1H-indazole-6-carboxylate (2.00 g, 11.4 mmol, 1.0 eq) and iodomethane (4.83 g, 34.1 mmol, 3.0 eq) in DMF (20 mL) was added cesium carbonate (7.40 g, 22.7 mmol, 2.0 eq) and the reaction mixture was stirred at room temperature for 2 h. After filtered, The filtration was diluted with EtOAc (100 mL) and washed with brine (30 mL×5). The organic layer was dried over anhydrous Na₂SO₄, filtered and concentrated under reduced pressure. The residue was purified by flash column chromatography on silica gel (PE/EA=1/1) to obtain methyl 1-methyl-1H-indazole-6-carboxylate (1.35 g, yield 62%) as a yellow solid and methyl 2-methyl-2H-indazole-6-carboxylate (800 mg, yield 37%) as a yellow oil.

Methyl 1-methyl-1H-indazole-6-carboxylate: LC-MS (ESI): mass calcd. for C₁₀H₁₀N₂O₂, 190.20; m/z found, 191.1 [M+H]⁺. ¹H NMR (400 MHz, DMSO-d₆) δ 8.30 (s, 3H), 8.17 (s, 1H), 7.92-7.84 (m, 1H), 7.70 (dd, J=8.4, 1.2 Hz, 1H), 4.14 (s, 3H), 3.91 (s, 3H).

Methyl 2-methyl-2H-indazole-6-carboxylate: LC-MS (ESI): mass calcd. for C₁₀H₁₀N₂O₂, 190.20; m/z found, 191.1 [M+H]⁺. ¹H NMR (400 MHz, DMSO-d₆) δ 8.46 (s, 1H), 8.28 (s, 1H), 7.81 (d, J=8.8 Hz, 1H), 7.56 (dd, J=8.8, 1.2 Hz, 1H), 4.23 (s, 3H), 3.88 (s, 3H).

### Step B: (1-methyl-1H-indazol-6-yl)methan-d₂-ol

To a solution of methyl 1-methyl-1H-indazole-6-carboxylate (700 mg, 3.68 mmol, 1.0 eq) in THF (20 mL) was added LiAlD₄ (155 mg, 3.68 mmol, 1.0 eq) in portions at 0° C. and the reaction mixture was stirred at 0° C. for 0.5 h. The reaction was diluted with EtOAc (50 mL), quenched with sodium sulfate decahydrate, and stirred for 0.5 h. After filtration, the filtrate was concentrated under reduced pressure to afford (1-methyl-1H-indazol-6-yl)methan-d₂-ol (600 mg, yield 99%) as a yellow solid. LC-MS (ESI): mass calcd. for C₉H₈D₂N₂O, 164.20; m/z found, 165.1 [M+H]⁺.

### Step C: 6-(bromomethyl-d₂)-1-methyl-1H-indazole

To a solution of (1-methyl-1H-indazol-6-yl)methan-d₂-ol (500 mg, 3.05 mmol, 1.0 eq) in DCM (5.0 mL) was added Triphenylphosphine (1.20 g, 4.57 mmol, 1.5 eq) and the mixed solution was stirred under the atmosphere at 50° C. for 5 min. Then a solution of CBr₄ (1.51 g, 4.57 mmol, 1.5 eq) in DCM (5.0 mL) was added dropwise to above mixture and the resulting reaction mixture was stirred at 50° C. for 2 h. After evaporation, the residue was purified by flash column chromatography on silica gel (PE/EA=2/1) to obtain 6-(bromomethyl-d₂)-1-methyl-1H-indazole (300 mg yield 43%) as a yellow solid. LC-MS (ESI): mass calcd. for C₉H₇D₂BrN₂, 226.01; m/z found, 227.0 [M+H]⁺.

### Intermediate B20: 4-(3-(bromomethyl-d₂)phenyl)-1-(methyl-d₃)-1H-pyrazole

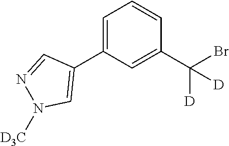 [SMILES: [2H]C([2H])(Br)c1cccc(-c2cnn(C([2H])([2H])[2H])c2)c1]

### Step A: 4-bromo-1-(methyl-d₃)-1H-pyrazole

To a solution of 4-bromo-1H-pyrazole (1.00 g, 6.80 mmol, 1.0 eq) in DMF (5.00 mL) were added Cs₂CO₃ (4.43 g, 13.6 mmol, 2.0 eq) and iodomethane-d₃ (635 μL, 10.2 mmol, 1.5 eq). The reaction mixture was stirred at room temperature for 16 h. After filtration, the mixture was diluted with EA (30 mL), washed with brine (20 mL×4), dried over anhydrous sodium sulfate, filtered and concentrate under reduced pressure to give 4-bromo-1-(methyl-d₃)-1H-pyrazole (1.10 g, yield 98%) as a yellow oil. LC-MS (ESI): mass calcd. for C₄H₂D₃BrN₂, 162.99; m/z found, 164.1 [M+H]⁺.

### Step B: methyl 3-(1-(methyl-d₃)-1H-pyrazol-4-yl)benzoate

To a solution of 4-bromo-1-(methyl-d₃)-1H-pyrazole (500 mg, 3.05 mmol, 1.0 eq), (3-(methoxycarbonyl) phenyl)boronic acid (658 mg, 3.66 mmol, 1.2 eq), and potassium carbonate (1.26 g, 9.15 mmol, 3.0 eq) in 1,4-Dioxane (10.0 mL) and water (0.10 mL) was added Pd(dppf)Cl₂ (249 mg, 305 μmol, 0.1 eq). The reaction mixture was stirred under N₂ at 100° C. for 2 h. After cooled to room temperature, the reaction mixture was filtered and the filtrate was concentrated under reduced pressure. The residue was purified by flash column chromatography on silica gel (EA/PE=1/1) to give methyl 3-(1-(methyl-d₃)-1H-pyrazol-4-yl) benzoate (400 mg, yield 60%) as a yellow oil. LC-MS (ESI): mass calcd. for C₁₂H₉D₃N₂O₂, 219.11; m/z found, 220.2 [M+H]⁺.

### Step C: (3-(1-(methyl-d₃)-1H-pyrazol-4-yl)phenyl)methan-d₂-ol

To a solution of methyl 3-(1-(methyl-d₃)-1H-pyrazol-4-yl) benzoate (400 mg, 1.82 mmol, 1.0 eq) in THF (10.0 mL) was added LiAlD₄ (153 mg, 3.65 mmol, 2.0 eq) in portions at 0° C. The reaction mixture was stirred at 0° C. for 40 min. The mixture was cautiously quenched with sodium sulfate decahydrate (500 mg) and filtered. The filtrate was concentrated under reduced pressure to give (3-(1-(methyl-d₃)-1H-pyrazol-4-yl)phenyl)methan-d₂-ol (300 mg, yield 85%) as a white solid. LC-MS (ESI): mass calcd. for C₁₁H₇D₅N₂O, 193.12; m/z found, 194.2 [M+H]⁺. ¹H NMR (400 MHz, CDCl₃) δ 7.69 (s, 1H), 7.54 (s, 1H), 7.41 (s, 1H), 7.34-7.25 (m, 2H), 7.14 (d, J=7.4 Hz, 1H).

### Step D: 4-(3-(bromomethyl-d₂)phenyl)-1-(methyl-d₃)-1H-pyrazole

To a solution of (3-(1-(methyl-d₃)-1H-pyrazol-4-yl) phenyl)methan-d₂-ol (300 mg, 1.55 mmol, 1.0 eq) and triphenylphosphine (611 mg, 2.33 mmol, 1.5 eq) in DCM (5.00 mL) was added CBr₄ (772 mg, 2.33 mmol, 1.5 eq) under N₂. The reaction mixture was stirred at 50° C. for 2 h. After evaporation, the residue was purified by flash column chromatography on silica gel (EA/PE=1/1) to give 4-(3-(bromomethyl-d₂)phenyl)-1-(methyl-d₃)-1H-pyrazole (350 mg, yield 88%) as a white solid. LC-MS (ESI): mass calcd. for C₁₁H₆D₅BrN₂, 255.04; m/z found, 256.2 [M+H]⁺. ¹H NMR (400 MHz, DMSO-d₆) δ 8.15 (s, 1H), 7.87 (s, 1H), 7.65 (s, 1H), 7.51 (d, J=7.6 Hz, 1H), 7.35 (t, J=7.6 Hz, 1H), 7.26 (d, J=7.6 Hz, 1H).

### Intermediate B21: 4-(3-(bromomethyl-d₂) phenyl)-1-(difluoromethyl)-1H-pyrazole

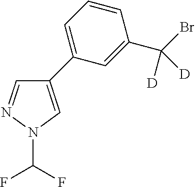 [SMILES: [2H]C([2H])(Br)c1cccc(-c2cnn(C(F)F)c2)c1]

### Step A: methyl 3-(1-(difluoromethyl)-1H-pyrazol-4-yl)benzoate

To a solution of 4-bromo-1-(difluoromethyl)-1H-pyrazole (500 mg, 2.54 mmol, 1.0 eq), (3-(methoxycarbonyl) phenyl) boronic acid (548 mg, 3.05 mmol, 1.2 eq), and Cesium carbonate (2.48 g, 7.61 mmol, 3.0 eq) in 1,4-Dioxane (20.0 mL) and Water (5.00 mL) was added 1,1′-Bis(diphenylphosphino)ferrocene-palladium(II) dichloride (186 mg, 254 μmol, 1.0 eq). The reaction mixture was stirred under nitrogen at 90° C. for 2 h. After cooled to room temperature, the mixture was filtered and the filtrate was concentrated under reduced pressure. The crude product was purified by flash column chromatography on silica gel (PE/EA=20/1) to provide methyl 3-(1-(difluoromethyl)-1H-pyrazol-4-yl)benzoate (580 mg, yield 90%) as a yellow oil. LC-MS (ESI): mass calcd. for C₁₂H₁₀F₂N₂O₂, 252.07; m/z found, 253.1 [M+H]⁺. ¹H NMR (400 MHz, DMSO-d₆) δ 8.87 (s, 1H), 8.37 (s, 1H), 8.24 (t, J=1.6 Hz, 1H), 8.02-7.97 (m, 1H), 7.89-7.87 (m, 1H), 7.86 (t, J=67.6 Hz, 1H), 7.58 (t, J=7.8 Hz, 1H), 3.89 (s, 3H).

### Step B: (3-(1-(difluoromethyl)-1H-pyrazol-4-yl) phenyl)methan-d₂-ol

To a solution of methyl 3-(1-(difluoromethyl)-1H-pyrazol-4-yl)benzoate (200 mg, 793 μmol, 1.0 eq) in dry THF (10.0 mL) was added LiAlD₄ (66.6 mg, 1.59 mmol, 2.0 eq) at 0° C. The reaction mixture was stirred at 0° C. for 20 min. The reaction mixture was cautiously quenched with Na₂SO₄·10H₂O and stirred for 30 min. After Filtration, the filtrate was diluted with water and extracted with ethyl acetate (25 mL×3). The combined organic extracts were washed with water (30 mL) and brine (30 mL), dried over anhydrous sodium sulfate, filtered and concentrated under reduced pressure. The crude product was purified by flash column chromatography on silica gel (PE/EA=4/1) to provide (3-(1-(difluoromethyl)-1H-pyrazol-4-yl)phenyl)methan-d₂-ol (158 mg, yield 88%) as a yellow oil. LC-MS (ESI): mass calcd. for C₁₁H₈D₂F₂N₂O, 226.09; m/z found, 227.1 [M+H]⁺. ¹H NMR (400 MHz, DMSO-d₆) δ 8.72 (s, 1H), 8.29 (s, 1H), 7.93 (t, J=67.6 Hz, 1H), 7.69-7.55 (m, 2H), 7.41 (d, J=7.6 Hz, 1H), 7.32-7.23 (m, 1H), 5.22 (s, 1H).

### Step C: 4-(3-(bromomethyl-d₂) phenyl)-1-(difluoromethyl)-1H-pyrazole

To a solution of (3-(1-(difluoromethyl)-1H-pyrazol-4-yl)phenyl)methan-d₂-ol (145 mg, 641 μmol, 1.0 eq) in DCM (10.0 mL) were added Triphenylphosphine (252 mg, 961 μmol, 1.5 eq) and carbon tetrabromide (319 mg, 961 μmol, 1.5 eq). The reaction mixture was stirred at 50° C. for 1 h. After cooled to room temperature, the reaction mixture was diluted with DCM (20 mL), washed with saturated aqueous sodium bicarbonate (20 mL), water (20 mL) and brine (20 mL), dried over anhydrous Na₂SO₄, filtered and concentrated under reduced pressure. The residue was purified by Prep-TLC (PE/EA=10/1) to provide 4-(3-(bromomethyl-d₂)phenyl)-1-(difluoromethyl)-1H-pyrazole (172 mg, yield 93%) as a white solid. LC-MS (ESI): mass calcd. for C₁₁H₇D₂BrF₂N₂, 288.00; m/z found, 289.0 [M+H]⁺. ¹H NMR (400 MHz, DMSO-d₆) δ 8.75 (s, 1H), 8.29 (s, 1H), 7.85 (t, J=59.2 Hz, 1H), 7.83 (d, J=7.6 Hz, 1H), 7.67-7.63 (m, 1H), 7.43-7.35 (m, 2H).

### Intermediate B22: 4-(3-(bromomethyl-d₂)phenyl)-1-methyl-1H-pyrazole

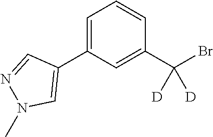 [SMILES: [2H]C([2H])(Br)c1cccc(-c2cnn(C)c2)c1]

### Step A: methyl 3-(1-methyl-1H-pyrazol-4-yl)benzoate

To a solution of 4-bromo-1-methyl-1H-pyrazole (6.00 g, 37.3 mmol, 1.0 eq), (3-(methoxycarbonyl)phenyl) boronic acid (8.05 g, 44.7 mmol, 1.2 eq), and potassium carbonate (15.5 g, 112 mmol, 3.0 eq) in 1,4-Dioxane (80.0 mL) and Water (8.00 mL) was added Pd(dppf)Cl₂ (1.52 g, 1.86 mmol, 0.05 eq). The reaction mixture was stirred under N₂ at 100° C. for 3 h. After cooled to room temperature, the reaction mixture was filtered and the filtrate was concentrated under reduced pressure. The residue was purified by flash column chromatography on silica gel (ethyl acetate in petroleum ether, 50% v/v) to give methyl 3-(1-methyl-1H-pyrazol-4-yl) benzoate (6.50 g, yield 80%) as a yellow oil. LC-MS (ESI): mass calcd. for C₁₂H₁₂N₂O₂, 216.09; m/z found, 217.1 [M+H]⁺.

### Step B: (3-(1-methyl-1H-pyrazol-4-yl)phenyl)methan-d₂-ol

To a solution of methyl 3-(1-methyl-1H-pyrazol-4-yl) benzoate (7.50 g, 34.7 mmol, 1.0 eq) in dry THF (50.0 mL) was added LiAlD₄ (2.18 g, 52.0 mmol, 1.5 eq) in portions at 0° C. The reaction mixture was stirred at 0° C. for 40 min. Then sodium sulfate decahydrate (5 g) was cautiously added to above mixture and filtered. The filtrate was concentrated under reduced pressure to give (3-(1-methyl-1H-pyrazol-4-yl)phenyl)methan-d₂-ol (5.30 g, yield 80%) as a white solid. LC-MS (ESI): mass calcd. for C₁₁H₁₀D₂N₂O, 190.11; m/z found, 191.1 [M+H]⁺.

¹H NMR (400 MHz, CDCl₃) δ 7.70 (s, 1H), 7.55 (s, 1H), 7.41 (s, 1H), 7.34-7.26 (m, 2H), 7.15 (d, J=7.4 Hz, 1H), 3.87 (s, 3H).

### Step C: 4-(3-(bromomethyl-d₂)phenyl)-1-methyl-1H-pyrazole

To a solution of (3-(1-methyl-1H-pyrazol-4-yl)phenyl)methan-d₂-ol (500 mg, 2.63 mmol, 1.0 eq) in DCM (10.0 mL) was added triphenylphosphine (1.03 g, 3.94 mmol, 1.5 eq) at room temperature. Then A solution of perbromo methane (1.31 g, 3.94 mmol, 1.5 eq) in DCM (3 mL) was added to above mixture under N₂ and the resulting mixture was stirred under N₂ at 50° C. for 2 h. After evaporation, the residue was purified by flash column chromatography on silica gel (ethyl acetate in petroleum ether, 17% v/v) to give 4-(3-(bromomethyl-d2)phenyl)-1-methyl-1H-pyrazole (490 mg, yield 74%) as a white solid. LC-MS (ESI): mass calcd. for C₁₀H₉D₂BrN₂, 252.02; m/z found, 253.2 [M+H]⁺.

### Intermediate B23: 3-(1-(methyl-d₃)-1H-pyrazol-4-yl)benzaldehyde

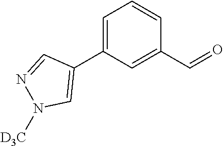 [SMILES: [2H]C([2H])([2H])n1cc(-c2cccc(C=O)c2)cn1]

### Step A: 4-bromo-1-(methyl-d₃)-1H-pyrazole

To a solution of 4-bromo-1H-pyrazole (3.00 g, 20.4 mmol, 1.0 eq) in DMF (60.0 mL) was added Cesium carbonate (13.3 g, 40.8 mmol, 2.0 eq) and the reaction mixture was stirred at room temperature for 1 h. Then CD₃I (5.92 g, 2.54 mL, 40.8 mmol, 2.0 eq) was dropwise added to above mixture and the resulting reaction mixture was stirred at room temperature overnight. The reaction mixture was diluted with EA (200 mL) and filtered. The filtrate was washed with brine (100 mL×5), dried over anhydrous Na₂SO₄, filtered and concentrated under reduced pressure to give 4-bromo-1-(methyl-d₃)-1H-pyrazole (2.40 g, yield 72%) as a yellow oil. LC-MS (ESI): mass calcd. for C₄H₂D₃BrN₂, 162.98; m/z found, 164.0 [M+H]⁺. ¹H NMR (400 MHz, DMSO-d₆) δ 7.85 (s, 1H), 7.44 (s, 1H).

### Step B: 3-(1-(methyl-d₃)-1H-pyrazol-4-yl)benzaldehyde

To a solution of (3-formylphenyl)boronic acid (2.00 g, 13.3 mmol, 1.0 eq), 4-bromo-1-(methyl-d₃)-1H-pyrazole (2.84 g, 17.3 mmol, 1.3 eq), and Cesium carbonate (13.0 g, 40.0 mmol, 3.0 eq) in 1,4-Dioxane (70.0 mL) and water (20.0 mL) was added 1,1′-bis(diphenyl phosphino)ferrocene-palladium(II) dichloride (976 mg, 1.33 mmol, 0.1 eq). The reaction mixture was stirred under nitrogen at 90° C. for 1 h. After cooled to room temperature, the mixture was filtered and the filtrate was concentrated under reduced pressure. The crude product was purified by flash column chromatography on silica gel (PE/EA=2/1) to provide 3-(1-(methyl-d₃)-1H-pyrazol-4-yl)benzaldehyde (1.85 g, yield 73%) as a yellow oil. LC-MS (ESI): mass calcd. for C₁₁H₇D₃N₂O, 189.10; m/z found, 190.1 [M+H]⁺. ¹H NMR (400 MHz, DMSO-d₆) δ 10.03 (s, 1H), 8.27 (s, 1H), 8.09 (s, 1H), 7.97 (s, 1H), 7.93-7.88 (m, 1H), 7.73 (d, J=7.6 Hz, 1H), 7.59 (t, J=7.6 Hz, 1H).

### Intermediate B24: 4-(4-formyl-1H-pyrazol-1-yl)benzonitrile

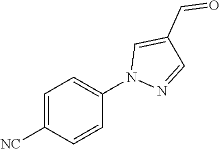 [SMILES: N#Cc1ccc(-n2cc(C=O)cn2)cc1]

To a solution of 1H-pyrazole-4-carbaldehyde (5.0 g, 52.0 mmol, 1.0 eq) in DMF (50 mL) was added NaH (60% suspend in oil) (1.5 g, 62.4 mmol, 1.2 eq) at 0° C. The reaction mixture was stirred at 0° C. for 0.5 h. 4-fluorobenzonitrile (6.9 g, 57.2 mmol, 1.1 eq) was added to above mixture and the reaction mixture was stirred at 25° C. for 16 h. The mixture was quenched with H₂O (100 mL) and extracted with EtOAc (50 mL×3). The organic layer was washed with brine (100 mL×4), dried over Na₂SO₄, filtered and concentrated under reduced pressure. The residue was purified by flash column chromatography on silica gel (PE/EA=2/1) to give 4-(4-formyl-1H-pyrazol-1-yl)benzonitrile (3.0 g, yield 29%) as a white solid. LC-MS (ESI): mass calcd. for C₁₁H₇N₃O, 197.06; m/z found, no Mass [M+H]⁺. ¹H NMR (400 MHz, DMSO-d₆) δ 9.94 (s, 1H), 9.41 (s, 1H), 8.37 (s, 1H), 8.15 (d, J=8.8 Hz, 2H), 8.05 (d, J=8.8 Hz, 2H).

### Intermediate B25: 2-oxoindoline-5-carbaldehyde

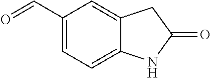 [SMILES: O=Cc1ccc2c(c1)CC(=O)N2]

### Step A: 5-vinylindolin-2-one

To a mixture of 5-bromoindolin-2-one (1.0 g, 4.72 mmol, 1.0 eq) and Potassium ethenyltrifluoroborate (948 mg, 7.07 mmol, 1.5 eq) in 1,4-Dioxane (20.0 mL) and H₂O (2.00 mL) were added Pd(PPh₃)₄ (545 mg, 472 μmol, 0.1 eq) and Na₂CO₃ (1.50 g, 14.1 mmol, 3.0 eq). The reaction mixture was stirred under N₂ at 100° C. for 4 h. After cooled to room temperature, the mixture was filtered and the filtrate was extracted with EtOAc (30 mL×3). The combined organic layers were washed with brine (50 mL), dried over anhydrous Na₂SO₄, filtered and concentrated under reduced pressure. The residue was purified by flash column chromatography on silica gel (PE/EtOAc=1/1) to give 5-vinylindolin-2-one (500 mg, yield 66%) as a brown solid. LC-MS (ESI): mass calcd. for C₁₀H₉NO, 159.07; m/z found, 160.1 [M+H]⁺. ¹H NMR (400 MHz, DMSO-d₆) δ 10.42 (s, 1H), 7.36 (s, 1H), 7.24 (d, J=8.0 Hz, 1H), 6.77 (d, J=8.0 Hz, 1H), 6.66 (dd, J=17.6, 10.8 Hz, 1H), 5.66 (d, J=17.6 Hz, 1H), 5.09 (d, J=10.8 Hz, 1H), 3.47 (s, 2H).

### Step B: 2-oxoindoline-5-carbaldehyde

Ozone was bubbled into a solution of 5-vinylindolin-2-one (200 mg, 1.26 mmol, 1.0 eq) in DCM (10.0 mL) at −78° C. for 1 h. On completion, excess Ozone was purged from the reaction mixture with nitrogen. Then dimethylsulfide (5 mL) was added to above mixture and the reaction mixture was stirred for 30 min. After evaporation, the residue was purified by flash column chromatography on silica gel (PE/EA=1/1) to give 2-oxoindoline-5-carbaldehyde (180 mg, yield 89%) as a light yellow solid. LC-MS (ESI): mass calcd. for C₉H₇NO₂, 161.05; m/z found, 162.2 [M+H]⁺.

### Intermediate B26: 1-((2-chlorophenyl)sulfonyl)-1H-pyrazole-4-carbaldehyde

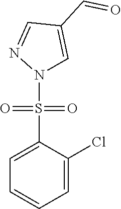 [SMILES: O=Cc1cnn(S(=O)(=O)c2ccccc2Cl)c1]

To a solution of 1H-pyrazole-4-carbaldehyde (500 mg, 5.20 mmol, 1.0 eq) and triethylamine (796 μL, 5.72 mmol, 1.1 eq) in DCM (5.00 mL) was added 2-chlorobenzenesulfonyl chloride (1.10 g, 5.20 mmol, 1.0 eq) in portions. The reaction mixture was stirred at room temperature for 2 h. After filtration, the mixture was concentrated under reduced pressure and the residue was purified by flash column chromatography on silica gel (EA/PE=1/10) to give 1-((2-chlorophenyl)sulfonyl)-1H-pyrazole-4-carbaldehyde (780 mg, yield 55%) as a yellow solid. LC-MS (ESI): mass calcd. for C₁₀H₇ClN₂O₃S, 269.99; m/z found, 271.2 [M+H]⁺. ¹H NMR (400 MHz, DMSO-d₆) δ 9.94 (s, 1H), 9.42 (s, 1H), 8.36-8.28 (m, 2H), 7.91-7.82 (m, 1H), 7.81-7.68 (m, 2H).

### Intermediate B27: 3-(1-methyl-1H-pyrazol-4-yl) benzaldehyde

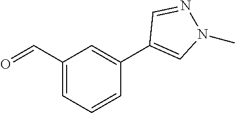

To a solution of 3-bromobenzaldehyde (5.00 g, 27.0 mmol, 1.0 eq), (1-methyl-1H-pyrazol-4-yl)boronic acid (4.08 g, 32.4 mmol, 1.2 eq), and Cesium carbonate (26.4 g, 81.1 mmol, 3.0 eq) in 1,4-Dioxane (100 mL) and Water (30.0 mL) was added 1,1′-Bis(diphenylphosphino)ferrocene-palladium(II) dichloride (1.98 g, 2.70 mmol, 0.1 eq). The reaction mixture was stirred under nitrogen at 95° C. for 2 h. After cooled to room temperature, the mixture was filtered and the filtrate was concentrated under reduced pressure. The crude product was purified by flash column chromatography on silica gel (PE/EA=1/1) to provide 3-(1-methyl-1H-pyrazol-4-yl)benzaldehyde (4.00 g, yield 79%) as a yellow oil. LC-MS (ESI): mass calcd. for C₁₁H₁₀N₂O, 186.08; m/z found, 187. [M+H]⁺. ¹H NMR (400 MHz, DMSO-d₆) δ 10.03 (s, 1H), 8.27 (s, 1H), 8.08 (s, 1H), 7.97 (s, 1H), 7.91 (d, J=7.6 Hz, 1H), 7.73 (d, J=7.6 Hz, 1H), 7.59 (t, J=7.6 Hz, 1H), 3.89 (s, 3H).

### Intermediate B28: 3-(1-(oxetan-3-yl)-1H-pyrazol-4-yl)benzaldehyde

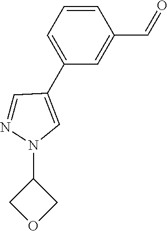 [SMILES: O=Cc1cccc(-c2cnn(C3COC3)c2)c1]

### Step A: 4-bromo-1-(oxetan-3-yl)-1H-pyrazole

A mixture of 4-bromo-1H-pyrazole (500 mg, 3.40 mmol, 1.0 eq), 3-iodooxetane (626 mg, 3.40 mmol, 1.0 eq), and Cesium carbonate (1.11 g, 3.40 mmol, 1.0 eq) in DMF (5.00 mL) was stirred at 100° C. for 18 h. After cooled to room temperature, the reaction mixture was quenched with water (30 mL) and extracted with DCM (40 mL×3). The organic layer was washed with brine (30 mL×4), dried over anhydrous Na₂SO₄, filtered and concentrated under reduced pressure to give 4-bromo-1-(oxetan-3-yl)-1H-pyrazole (620 mg, yield 89%) as a yellow solid. LC-MS (ESI): mass calced for: C₆H₇BrN₂O 203.04; m/z found, No mass signal.

¹H NMR (400 MHz, DMSO-d₆) δ 7.95 (s, 1H), 7.69 (s, 1H), 5.59-5.51 (m, 1H), 4.91-4.82 (m, 4H).

### Step B: 3-(1-(oxetan-3-yl)-1H-pyrazol-4-yl)benzaldehyde

A mixture of 4-bromo-1-(oxetan-3-yl)-1H-pyrazole (620 mg, 3.05 mmol, 1.0 eq), (3-formylphenyl)boronic acid (916 mg, 6.11 mmol, 2.0 eq), Cs₂CO₃ (1.99 g, 6.11 mmol, 2.0 eq), and 1,1′-Bis(diphenylphosphino)ferrocene-palladium(II) dichloride (223 mg, 305 μmol, 0.1 eq) in 1,4-Dioxane (10.0 mL) and H₂O (1.00 mL) was stirred under N₂ atmosphere at 95° C. for 18 h. After cooled to room temperature, the reaction mixture was quenched with water (10 mL) and extracted with EtOAc (30 mL×3). The organic layer was washed with brine (20 mL), dried over anhydrous Na₂SO₄, filtered and concentrated under reduced pressure. The residue was purified by flash column chromatography on silica gel (methanol in dichloromethane, from 0 to 5%) to afford 3-(1-(oxetan-3-yl)-1H-pyrazol-4-yl)benzaldehyde (500 mg, yield 68%) as a yellow solid. LC-MS (ESI): mass calced for: C₁₃H₁₂N₂O₂ 228.25; m/z found, 229.1 [M+H]⁺. ¹H NMR (400 MHz, DMSO-d₆) δ 10.04 (s, 1H), 8.53 (s, 1H), 8.15 (s, 2H), 7.96 (d, J=7.8 Hz, 1H), 7.76 (d, J=7.8 Hz, 1H), 7.61 (t, J=7.8 Hz, 1H), 5.65-5.57 (m, 1H), 4.98-4.91 (m, 4H).

### Intermediate B29: 3-oxoisoindoline-5-carbaldehyde

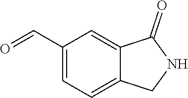 [SMILES: O=Cc1ccc2c(c1)C(=O)NC2]

The intermediate was prepared according to the procedure described in Journal of Pharmaceutical Science & Technology (2010), 2(12), 380-390 as a white solid. The analytical data is consistent with the report in the literature.

### Intermediate B30a: 1-methyl-1H-indazole-6-carbaldehyde

### Intermediate B30b:2-methyl-2H-indazole-6-carbaldehyde

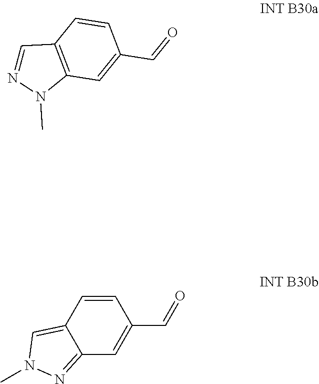

To a solution of 1H-indazole-6-carbaldehyde (1.00 g, 6.84 mmol, 1.0 eq) and K₂CO₃ (1.89 g, 13.7 mmol, 2.0 eq) in DMF (10.0 mL) was added MeI (1.46 g, 10.3 mmol, 1.5 eq) and the mixture was stirred at 30° C. for 2 h. The reaction mixture was diluted with H₂O (30 mL) and washed with brine and extracted with EA (30 ml×3). The organic phase was washed with brine (30 mL×4), dried over anhydrous Na₂SO₄, filtered and concentrated under reduced pressure. The residue was purified by flash column chromatography on silica gel (ethyl acetate in petroleum ether, from 0% to v/v) to give 1-methyl-1H-indazole-6-carbaldehyde (600 mg, yield 54%) as a yellow solid and 2-methyl-2H-indazole-6-carbaldehyde (360 mg, yield 32%).

INT B30a, 1-methyl-1H-indazole-6-carbaldehyde: LC-MS (ESI) mass calced for: C₉H₈N₂O, 160.06; m/z found, 161.1 [M+H]⁺. ¹H NMR (400 MHz, DMSO-d₆) δ 10.14 (s, 1H), 8.33 (s, 1H), 8.21 (s, 1H), 7.93 (d, J=8.4 Hz, 1H), 7.63 (d, J=8.4 Hz, 1H), 4.16 (s, 3H).

INT B30b: 2-methyl-2H-indazole-6-carbaldehyde: LC-MS (ESI) mass calced for: C₉H₈N₂O, 160.06; m/z found, 161.1 [M+H]⁺. ¹H NMR (400 MHz, DMSO-d₆) δ 10.05 (s, 1H), 8.49 (s, 1H), 8.32 (s, 1H), 7.85 (d, J=8.6 Hz, 1H), 7.47 (d, J=8.6 Hz, 1H), 4.25 (s, 3H).

### Intermediate B31a: 1-methyl-1H-indazole-7-carbaldehyde

### Intermediate B31b: 2-methyl-2H-indazole-7-carbaldehyde

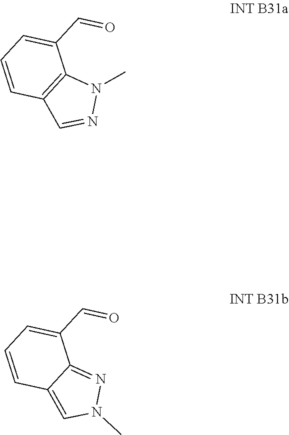

To a solution of 1H-indazole-7-carbaldehyde (300 mg, 2.05 mmol, 1.0 eq) in DMF (6.00 mL) were added potassium carbonate (851 mg, 6.16 mmol, 3.0 eq) and iodomethane (583 mg, 4.11 mmol, 2.0 eq). The reaction was stirred under N₂ atmosphere at room temperature for 2 h. After filtration, the filtrate was diluted with H₂O (15 mL) and extracted with EA (15 mL×3). The organic layer was washed with brine (15 mL×4), dried over anhydrous sodium sulfate, filtered and concentrated under reduced pressure. The residue was purified by Prep-HPLC with YMC-Actus Triart C18 (5 um, 20×250 mm), and mobile phase of 5-95% ACN in water (0.1% TFA) over 20 min and then hold at 100% ACN for 2 min, at a flow rate of 25 mL/min to obtained 2-methyl-2H-indazole-7-carbaldehyde (60 mg, yield 11%) as a yellow solid and 1-methyl-1H-indazole-7-carbaldehyde (110 mg, yield 20.1%) as a white solid.

INT B31a, 2-methyl-2H-indazole-7-carbaldehyde: LC-MS (ESI): mass calced for: C₉H₈N₂O 160.18; m/z found, 161.1 [M+H]⁺. ¹H NMR (400 MHz, DMSO-d₆) δ 10.46 (s, 1H), 8.59 (s, 1H), 8.12 (d, J=8.2 Hz, 1H), 7.87 (d, J=7.0 Hz, 1H), 7.25 (t, J=7.6 Hz, 1H), 4.25 (s, 3H).

INT B31b: 1-methyl-1H-indazole-7-carbaldehyde: LC-MS (ESI): mass calced for: C₉H₈N₂O 160.18; m/z found, 161.1 [M+H]⁺. ¹H NMR (400 MHz, DMSO-d₆) δ 10.30 (s, 1H), 8.25 (s, 1H), 8.15 (dd, J=8.0, 1.0 Hz, 1H), 8.04 (dd, J=7.2, 1.0 Hz, 1H), 7.43-7.25 (m, 1H), 4.36 (s, 3H).

### Intermediate B32a: 1-methyl-1H-indazole-5-carbaldehyde

### Intermediate B32b: 2-methyl-2H-indazole-5-carbaldehyde

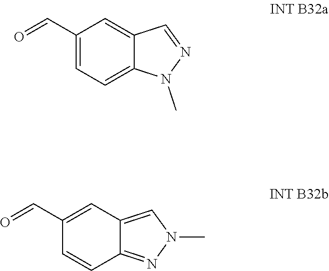

To a solution of 1H-indazole-5-carbaldehyde (1.0 g, 6.8 mmol, 1.0 eq) in DMF (20.0 mL) was added Potassium carbonate (2.8 g, 20.5 mmol, 3.0 eq) at 0° C. The reaction mixture was stirred at 0° C. for 10 min. Then methyl iodide (1.9 g, 13.7 mmol, 2.0 eq) was dropwise added to above mixture at 0° C. The mixture was stirred at 25° C. for 50 min. The reaction mixture was diluted with EA (150 mL) and filtered. The filtrate was washed with brine (60 mL×5), dried over anhydrous Na₂SO₄, filtered and concentrated under reduced pressure. The residue was purified by flash column chromatography on silica gel (EA/PE=1/5 to 1/2) to give 1-methyl-1H-indazole-5-carbaldehyde (0.8 g, yield 72%) as a yellow solid and 2-methyl-2H-indazole-5-carbaldehyde (0.3 g, 28%) as a white solid.

INT B32a, 1-methyl-1H-indazole-5-carbaldehyde: LC-MS (ESI): mass calcd. for C₉H₈N₂O, 160.08; m/z found, 161.2 (M+H)⁺. ¹H NMR (400 MHz, DMSO-d₆) δ 10.03 (s, 1H), 8.42 (s, 1H), 8.32 (s, 1H), 7.87 (dd, J=8.8, 1.2 Hz, 1H), 7.78 (d, J=8.8 Hz, 1H), 4.10 (s, 3H).

INT B32b, 2-methyl-2H-indazole-5-carbaldehyde: LC-MS (ESI): mass calcd. for C₉H₈N₂O, 160.08; m/z found, 161.2 (M+H)⁺. ¹H NMR (400 MHz, DMSO-d₆) δ 10.01 (s, 1H), 8.73 (s, 1H), 8.48 (s, 1H), 7.74-7.69 (m, 2H), 4.26 (s, 3H).

### Intermediate B33a: 1-methyl-1H-indazole-4-carbaldehyde

### Intermediate 33b: 2-methyl-2H-indazole-4-carbaldehyde

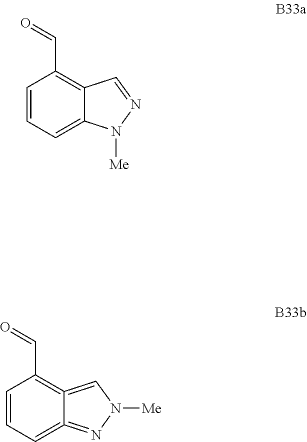

To a stirred mixture of 1-methyl-1H-indazole-4-carbaldehyde (200 mg, 1.36 mmol, 1.0 eq) in DMF (5.00 mL) were added K₂CO₃ (188 mg, 1.36 mmol, 2.0 eq) and MeI (193 mg, 85.0 μL, 1.36 mmol, 1.0 eq). The resulting mixture was stirred at 25° C. for 2 h. The reaction mixture was diluted with H₂O (50 mL) and extracted with EtOAc (30 mL×3). The combined organic layers were washed with brine (50 mL×3), dried over anhydrous Na₂SO₄, filtered and concentrated under reduced pressure. The residue was purified by flash column chromatography on silica gel (PE/EtOAc=1/1) to give 2-methyl-2H-indazole-4-carbaldehyde (80.0 mg, yield 36%) as a white solid and 1-methyl-1H-indazole-4-carbaldehyde (110 mg, yield 50%) as a white solid.

INT 33a: 1-methyl-1H-indazole-4-carbaldehyde: LC-MS (ESI): mass calcd. for C₉H₈N₂O, 160.06; m/z found, 161.1 [M+H]⁺. ¹H NMR (400 MHz, DMSO-d₆) δ 10.26 (s, 1H), 8.52 (s, 1H), 8.12 (d, J=8.4 Hz, 1H), 7.91 (d, J=7.0 Hz, 1H), 7.70 (dd, J=8.4, 7.0 Hz, 1H), 4.18 (s, 3H).

INT 33b: 2-methyl-2H-indazole-4-carbaldehyde: LC-MS (ESI): mass calcd. for C₉H₈N₂O, 160.06; m/z found, 161.1 [M+H]⁺. ¹H NMR (400 MHz, DMSO-d₆) δ 10.11 (s, 1H), 8.80 (s, 1H), 8.03 (d, J=8.6 Hz, 1H), 7.84 (t, J=7.0 Hz, 1H), 7.50 (dd, J=8.6, 7.0 Hz, 1H), 4.27 (s, 3H).

### Intermediate B34: 1-((1,1-dioxidotetrahydro-2H-thiopyran-4-yl)methyl)-1H-pyrazole-4-carbaldehyde

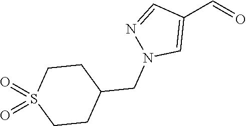 [SMILES: O=Cc1cnn(CC2CCS(=O)(=O)CC2)c1]

### Step A: (1,1-dioxidotetrahydro-2H-thiopyran-4-yl)methyl methanesulfonate

To a solution of 4-(hydroxymethyl)tetrahydro-2H-thiopyran 1,1-dioxide (200 mg, 1.22 mmol, 1.0 eq) and TEA (185 mg, 255 μL, 1.83 mmol, 1.5 eq) in DCM (5.00 mL) was added dropwise MsCl (181 mg, 123 μL, 1.58 mmol, 1.3 eq) under N₂ at 0° C. and the mixture was stirred at 0° C. for 2 h. The mixture was poured into water (6 mL) and extracted with DCM (20 mL×3). The organic layer was washed with brine (20 mL×4), dried over anhydrous MgSO₄, and filtered. The fitrate was concentrated under reduced pressure to give crude product (1,1-dioxidotetrahydro-2H-thiopyran-4-yl)methyl methanesulfonate (150 mg, yield 52%) as a white solid. The crude product was directly used in next step without further purification. LC-MS (ESI): mass calcd. for C₇H₁₄O₅S₂, 242.02; m/z found, no Mass.

### Step B: 1-((1,1-dioxidotetrahydro-2H-thiopyran-4-yl)methyl)-1H-pyrazole-4-carbaldehyde

To a solution of (1,1-dioxidotetrahydro-2H-thiopyran-4-yl)methyl methanesulfonate (150 mg, 619 gmol, 1.0 eq) and Cs₂CO₃ (504 mg, 1.55 mmol, 2.5 eq) in DMF (5.0 mL) was added 1H-pyrazole-4-carbaldehyde (71.4 mg, 743 μmol, 1.2 eq). The mixture was stirred at 25° C. for 16 h. After filtration, the filtrate was diluted with water (15 mL) and extracted with EtOAc (10 mL×3). The organic layer was washed with brine (15 mL×4), dried over anhydrous MgSO₄, filtered and concentrated under reduced pressure. The residue was purified by Prep-TLC (DCM/MeOH=10/1) to obtain 1-((1,1-dioxidotetrahydro-2H-thiopyran-4-yl)methyl)-1H-pyrazole-4-carbaldehyde (50.0 mg, yield 30%) as a yellow solid. LC-MS (ESI): mass calcd. for C₁₀H₁₄N₂O₃S, 242.07; m/z found, 243.0 [M+H]⁺. ¹1H NMR (400 MHz, DMSO-d₆) δ 9.80 (s, 1H), 8.46 (s, 1H), 8.03 (s, 1H), 4.17 (d, J=7.2 Hz, 2H), 3.12-3.07 (m, 4H), 1.85-1.82 (m, 2H), 1.70-1.60 (m, 3H).

### Intermediate B35: benzyl 4-(chloromethyl)-3,6-dihydropyridine-1(2H)-carboxylate

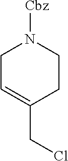 [SMILES: O=C(OCc1ccccc1)N1CC=C(CCl)CC1]

### Step A: benzyl 4-methylenepiperidine-1-carboxylate

A solution of tert-butyl 4-methylenepiperidine-1-carboxylate (25 g, 126.9 mmol) was stirred in TFA/CH₂Cl₂ (v/v=1:5) for 2 h at room temperature and concentrate in vacuo. The corresponding amine (12.3 g, 126.8 mmol, 1.0 equiv) was dissolved in THF and K₂CO₃ (52.6 g, 380.4 mmol, 3.0 equiv) and benzyl chloroformate (19.6 mL, 139.5 mmol, 1.10 equiv) were added. The reaction mixture was allowed to stir at 23° C. overnight. To the resulting suspension was added with sat aq. NaHCO₃ solution and the aqueous phase was extracted with CH₂Cl₂. The combined organic layers were dried over Na₂SO₄, filtered and concentrated in vacuo. The crude products were purified by column chromatography on silica gel to give the title compound (19.0 g, 76.9 mmol, 80% yield).

¹H NMR (400 MHz, CDCl₃): δ 7.40-7.27 (m, 5H), 5.15 (s, 2H), 4.76 (s, 2H), 3.51 (m, 4H), 2.20 (m, 4H).

### Step B: benzyl 2-hydroxy-4-methylenepiperidine-1-carboxylate

Selenium dioxide (7.9 g, 71.4 mmol, 0.55 eq) was suspended in DCM (750 mL), before t-butyl-hydroperoxide (24.6 g, 272.8 mmol, 2.10 eq) was added and the mixture was stirred for 30 min at 0° C. Benzyl 4-methylenepiperidine-1-carboxylate (30.0 g, 129.9 mmol, 1 eq) in DCM (100 mL) was added to the mixture and stirred for 1 hr at 0° C., and further 18 h at 20° C. Ice chips and 10% w/v sodium bisulfite (450 mL) was added to the solution. The aqueous layer was extracted with DCM (150 mL×3) and the combined organic layer was washed with brine (200 mL×2), dried over anhydrous Na₂SO₄, filtered and concentrated in vacuo. The crude product was purified through silica gel column chromatography (ethyl acetate in petroleum ether). The desired product benzyl 2-hydroxy-4-methylenepiperidine-1-carboxylate (19.0 g, 76.9 mmol, 50% yield) was obtained as a light yellow oil.

¹H NMR (400 MHz, CDCl₃): δ 7.44-7.29 (m, 5H), 5.15 (s, 2H), 5.04 (s, 1H), 4.89 (d, J=1.9 Hz, 1H), 4.11 (s, 1H), 3.78 (m, 1H), 3.58 (s, 1H), 3.45-3.31 (m, 2H), 2.56-2.06 (m, 2H).

### Step C: benzyl 4-(chloromethyl)-3,6-dihydropyridine-1(2H)-carboxylate

The solution of benzyl 2-hydroxy-4-methylenepiperidine-1-carboxylate (19 g, 76.9 mmol, 1 eq) and K₂CO₃ (11.7 g, 84.6 mmol, 1.10 eq) in toluene (225 mL) was degassed and purged with Ar. SOCl₂ (10.1 g, 84.6 mmol, 1.10 eq) was added in one portion at 0° C., before the reaction temperature was increased to 40° C. and stirred for further 40 min. The reaction mixture was cooled to 0° C. and washed with pre-cooled (0° C.) 1N HCl (150 mL×2), HCl (0.1 N, 150 mL×2), H₂O (150 mL×2), brine (150 mL×2) and dried with Na₂SO₄, filtered and concentrated in vacuo. Benzyl 4-(chloromethyl)-3,6-dihydropyridine-1(2H)-carboxylate (16 g, 60.4 mmol, 70% yield) was obtained as a yellow oil.

¹H NMR (400 MHz, CDCl₃): δ 7.39-7.27 (m, 5H), 5.77 (d, J=23.1 Hz, 1H), 5.15 (s, 2H), 4.01 (m, 4H), 3.62 (m, 2H), 2.23 (m, 2H).

### Intermediate B36: tert-butyl (S)-5-amino-4-(4-fluoro-6-oxo-6,8-dihydro-2H,7H-spiro[furo[2,3-e]isoindole-3,4′-piperidin]-7-yl)-5-oxopentanoate

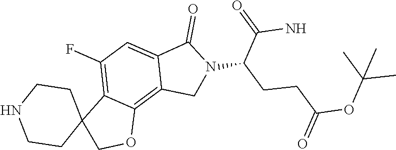 [SMILES: CC(C)(C)OC(=O)CC[C@@H](C(N)=O)N1Cc2c(cc(F)c3c2OCC32CCNCC2)C1=O]

### Step A: 4-bromo-3-fluoro-5-methoxybenzoic acid

CH₃ONa (30% in MeOH, 1.6 g, 20 eq.) at rt. was added to a solution of 4-bromo-3,5-difluorobenzoic acid (1.0 g, 10 eq.) in DMF (30 mL). The reaction mixture was stirred overnight at 60° C. After cooled to room temperature and ice water (150 mL) was added, then was treated with 2N HCl until pH=3-4. The reaction was extracted with EtOAc for three times, washed with brine and dried with anhydrous sodium sulfate. And concentrated to afford the title compound (1.0 g) as a solid, which was used in next step without further purification.

### Step B: 4-bromo-3-fluoro-5-hydroxybenzoic acid

To a solution of 4-bromo-3-fluoro-5-methoxybenzoic acid (1.0 g, 1.0 eq.) in dry DCM (40 mL) was cooled down to −78° C., then added BBr₃ (5.4 g, 5.0 eq.) dropwise during 10 min and stirred at −78° C. for 1 h. The reaction mixture was stirred overnight at rt. After was cooled down to −78° C., then added saturated ammonium chloride solution dropwise during 30 min. And concentrated to afford the title compound (0.9 g), which was used in next step without further purification.

### Step C: methyl 4-bromo-3-fluoro-5-hydroxybenzoate

Conc. H₂SO₄ (0.6 g, 1.5 eq.) at rt. was added to a solution of 4-bromo-3-fluoro-5-hydroxybenzoic acid (0.9 g, 1.0 eq.) in MeOH (30 mL). The reaction mixture was stirred overnight at 70° C. After cooled to room temperature and ice water (100 mL) was added. The reaction was extracted with EtOAc for three times, washed with brine and dried with anhydrous sodium sulfate. And concentrated to afford the title compound 4 (1.0 g) as a solid, which was used in next step without further purification.

### Step D: methyl 4-bromo-5-fluoro-2-formyl-3-hydroxybenzoate

To a solution of methyl 4-bromo-3-fluoro-5-hydroxybenzoate (249 mg, 1 mmol, 1 eq.) in TFA (5 mL) was added HMTA (560 mg, 4 mmol, 4 eq.) at 20° C. The mixture was stirred at 125° C. for 12 h. TLC (Petroleum ether/Ethyl acetate=5/1) indicated starting materials was consumed completely and there was desired product. The mixture was quenched with 2N HCl (5 V) and a yellow solid formed. The mixture was stirred for 10 mins and then additional water (5 V) was added and stirred for 1 hr. The mixture was filtered. The filter cake was dissolved in DCM and filtered on celite, dried and then remove most of the solvent in vacuo. The title compound (138 mg, 0.5 mmol, 50% yield) was obtained as a gray solid, which was indicated by 1H NMR.

¹H NMR (600 MHz, CDCl₃) δ 13.30 (s, 1H), 10.61 (s, 1H), 7.31 (d, J=8.5 Hz, 1H), 3.97 (s, 3H). LC-MS (m/z): [M−H]⁺=274.99.

### Step E: tert-butyl (S)-5-amino-4-(5-bromo-6-fluoro-4-hydroxy-1-oxoisoindolin-2-yl)-5-oxopentanoate

tert-Butyl (S)-4,5-diamino-5-oxopentanoate (212 mg, 1.05 mmol, 1.05 eq, HCl) was added in MeOH (5 mL) at 20° C., then DIEA (1.05 mmol, 1.05 eq), methyl 4-bromo-5-fluoro-2-formyl-3-hydroxybenzoate (277 mg, 1 mmol, 1 eq) and AcOH (1.5 mmol, 1.5 eq) were added to the mixture at the same temperature. After 1.5 h, add NaBH₃CN (2 mmol, 2 eq) to the mixture in portions and stirred the mixture at 20° C. for 3 h. After the reaction completed, the reaction mixture was quenched by addition H₂O at 20° C., and then concentrated under reduced pressure to remove MeOH. Then the mixture was extracted with EtOAc, the combined organic layers were washed with brine (200 mL×2), dried over Na₂SO₄, filtered and concentrated under reduced pressure to give a residue. The residue was purified by column chromatography (SiO₂, Petroleum ether/Ethyl acetate=1/1 to Ethyl acetate). The title compound (202 mg, 0.47 mmol, 47% yield) was obtained as a yellow solid. LC-MS (m/z): [M+H]⁺=453.28.

### Step F: Synthesis of benzyl (S)-4-(((2-(1-amino-5-(tert-butoxy)-1,5-dioxopentan-2-yl)-5-bromo-6-fluoro-1-oxoisoindolin-4-yl)oxy)methyl)-3,6-dihydropyridine-1(2H)-carboxylate

tert-Butyl (S)-5-amino-4-(5-bromo-6-fluoro-4-hydroxy-1-oxoisoindolin-2-yl)-5-oxopentanoate (202 mg, 0.47 mmol, 1 eq), benzyl 4-(chloromethyl)-3,6-dihydropyridine-1(2H)-carboxylate (Compound A, 280 mg, 0.49 mmol, 1.05 eq) and K₂CO₃ (194 mg, 1.41 mmol, 3 eq) were added in DMF (5 mL) at 20° C. Then stir the mixture at 60° C. for 12 h. After the reaction completed, the mixture was concentrated under reduced pressure to give a residue, which was added water (20 mL). The product was extracted with DCM (20 mL×3). The organic layers were dried over Na₂SO₄ and concentrated under reduced pressure to give a residue. The residue was purified by column chromatography on silica gel to afford the title compound as a yellow solid (204 mg, 66% yield). LC-MS (m/z): [M+H]⁺=682.40.

### Step G: benzyl (S)-7-(1-amino-5-(tert-butoxy)-1,5-dioxopentan-2-yl)-4-fluoro-6-oxo-7,8-dihydro-2H,6H-spiro[furo[2,3-e]isoindole-3,4′-piperidine]-1′-carboxylate

Benzyl (S)-7-(1-amino-5-(tert-butoxy)-1,5-dioxopentan-2-yl)-4-fluoro-6-oxo-7,8-dihydro-2H,6H-spiro[furo[2,3-e]isoindole-3,4′-piperidine]-1′-carboxylate (660 mg, 1 mmol, 1 eq), Bu₃SnH (1228 mg, 4 mmol) and AIBN (16.4 mg, 0.1 mmol, 0.1 eq) were added in toluene (5 mL) at 20° C. and then stir the mixture at 110° C. for 12 h. After the reaction completed, the mixture was quenched by addition saturated potassium fluoride solution and stirred for 1 h. The product was extracted with EA. The organic layers were dried over Na₂SO₄ and concentrated under reduced pressure to give a residue. The residue was purified by column chromatography on silica gel to afford the title compound as a yellow solid (400 mg, 69% yield).

¹H NMR (400 MHz, CDCl₃) δ 7.38-7.30 (m, 5H), 7.04 (d, J=8.4 Hz, 1H), 6.50 (s, 1H), 5.62 (s, 1H), 5.16 (s, 2H), 4.87 (dd, J=8.9, 6.1 Hz, 1H), 4.56 (s, 2H), 4.47 (d, J=17.2 Hz, 1H), 4.35 (s, 1H), 4.25 (s, 2H), 2.84 (s, 2H), 2.37-2.16 (m, 6H), 1.75 (m, 2H), 1.40 (s, 9H).

### Step H: tert-butyl (S)-5-amino-4-(4-fluoro-6-oxo-6,8-dihydro-2H,7H-spiro[furo[2,3-e]isoindole-3,4′-piperidin]-7-yl)-5-oxopentanoate

To a 100 mL flask equipped with a magnetic stirring bar was added benzyl (S)-7-(1-amino-5-(tert-butoxy)-1,5-dioxopentan-2-yl)-4-fluoro-6-oxo-7,8-dihydro-2H,6H-spiro[furo[2,3-e]isoindole-3,4′-piperidine]-1′-carboxylate (200 mg), MeOH (10 mL), and then added 10% Pd/C (20 mg). Followed by flushing flask with hydrogen, a balloon with hydrogen was attached and the reaction mixture was stirred for 2 h. Upon full consumption of the starting material by TLC monitoring (DCM:MeOH=10:1), the reaction mixture was filtered through celite and washed through with additional MeOH. Solvent was removed and the residual tert-butyl (S)-5-amino-4-(4-fluoro-6-oxo-6,8-dihydro-2H,7H-spiro[furo[2,3-e]isoindole-3,4′-piperidin]-7-yl)-5-oxopentanoate (120 mg) as a white solid was used directly in the next step.

### Intermediate B37: tert-butyl (S)-5-amino-5-oxo-4-(6′-oxo-1′,2′,6′,8′-tetrahydro-7′H-spiro[piperidine-4,3′-pyrrolo[3,4-g]indol]-7′-yl)pentanoate

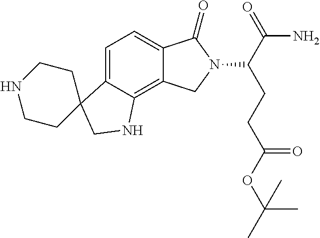

### Step A: 4-bromo-2-methyl-3-nitrobenzoic acid

Fuming nitric acid (20 mL) was added dropwise to a 0° C. solution of 4-bromo-2-methylbenzoic acid (20.0 g, 93.4 mmol) in concentrated sulfuric acid (80 mL). The reaction mixture was stirred at 55° C. overnight and poured onto ice (˜400 mL) and was extracted with EtOAc (3×100 mL). The extracts were washed with brine (2×50 mL), dried over NaSO₄, filtered, and concentrated to give compound 2 (25.0 g, crude). The material was used without further purification.

### Step B: methyl 4-bromo-2-methyl-3-nitrobenzoate

Concentrated sulfuric acid (16.4 g, 167 mmol) was added dropwise to a 0° C. solution of 4-bromo-2-methyl-3-nitrobenzoic acid (25.0 g, crude) in MeOH (150 mL). The reaction mixture was stirred at 90° C. overnight, solvent was removed under vacuum, EtOAc (100 mL) and sat. aq. NaHCO₃ (200 mL) was added. The products were extracted with EtOAc (50 mL×3), and the combined organic extracts were washed with brine, dried over NaSO₄, filtered, and concentrated in vacuo to give the title compound (12.0 g, crude). The residue was used in the next step without further purification.

### Step C: methyl 4-bromo-2-(bromomethyl)-3-nitrobenzoate

To a solution of 3 (12.0 g, 44.0 mmol) in DCE (200 mL) were added NBS (10.5 g, 58.8 mmol) and AIBN (0.722 g, 4.40 mmol) at room temperature. The mixture was stirred at 90° C. under N₂ for 6 h. The mixture was poured into aq. Na₂S₂O₃ at room temperature and extracted with EtOAc. The organic layer was separated, washed with satd. NaHCO₃ and brine, dried over Na₂SO₄, and concentrated in vacuo. The residue was purified by column chromatography to give the title compound (2.90 g) as a white solid.

¹HNMR (400 MHz, DMSO-d₆) δ 8.08 (d, J=8.5 Hz, 1H), 8.02 (d, J=8.5 Hz, 1H), 4.82 (s, 2H), 3.92 (s, 3H).

### Step D: tert-butyl 5-amino-4-(5-bromo-4-nitro-1-oxoisoindolin-2-yl)-5-oxopentanoate

To a solution of methyl 4-bromo-2-(bromomethyl)-3-nitrobenzoate (2.90 g, 8.22 mmol) in ACN (50 mL) was added tert-butyl (S)-4,5-diamino-5-oxopentanoate (2.94 g, 12.3 mmol) and DIEA (3.18 g, 24.7 mmol). The resulting mixture was stirred at 90° C. overnight, solvent was removed under vacuum, EtOAc (30 mL) and H₂O (50 mL) was added. The products were extracted with EtOAc (30 mL×3), and the combined organic extracts were washed with brine, dried over NaSO₄, filtered, and concentrated. The residue was purified by column chromatography to give the title compound (2.48 g).

### Step E: tert-butyl 5-amino-4-(4-amino-5-bromo-1-oxoisoindolin-2-yl)-5-oxopentanoate

To a solution of tert-butyl 5-amino-4-(5-bromo-4-nitro-1-oxoisoindolin-2-yl)-5-oxopentanoate (2.48 g, 5.62 mmol) in EtOH/H₂O (40 mL/4 mL) was added Fe (1.57 g, 28.1 mmol) and NH₄Cl (0.894 g, 16.9 mmol). The resulting mixture was stirred at 70° C. overnight then filtered through glass fiber filter paper on a Buchner funnel to remove the iron. The solid was rinsed with EtOH and the filtrate was concentrated and partitioned between EtOAc and H₂O (50 mL/50 mL). The aqueous layer was separated and washed with EtOAc (2×50 mL). The combined organic extracts were washed with brine (50 mL), dried over NaSO₄, filtered and concentrated. The residue was purified by column chromatography to give the title compound (1.74 g).

¹H NMR (600 MHz, DMSO) δ 7.55 (s, 1H), 7.48 (d, J=7.9 Hz, 1H), 7.18 (s, 1H), 6.83 (d, J=7.9 Hz, 1H), 5.66 (s, 2H), 4.71 (dd, J=10.6, 3.8 Hz, 1H), 4.47 (d, J=17.8 Hz, 1H), 4.26 (d, J=17.8 Hz, 1H), 2.21-2.11 (m, 2H), 1.33 (s, 9H).

### Step F: benzyl 4-(((2-(1-amino-5-(tert-butoxy)-1,5-dioxopentan-2-yl)-5-bromo-1-oxoisoindolin-4-yl)amino)methyl)-3,6-dihydropyridine-1(2H)-carboxylate

To a solution of tert-butyl 5-amino-4-(4-amino-5-bromo-1-oxoisoindolin-2-yl)-5-oxopentanoate (0.840 g, 2.04 mmol) in CH₃CN (20 mL) was added benzyl 4-(chloromethyl)-3,6-dihydropyridine-1(2H)-carboxylate (0.812 g, 3.07 mmol), DIEA (0.789 g, 6.12 mmol) and KI (0.510 g, 3.07 mmol). The resulting mixture was stirred at 70° C. overnight, solvent was removed under vacuum, EtOAc (10 mL) and H₂O (50 mL) was added. The products were extracted with EtOAc (20 mL×3), and the combined organic extracts were washed with brine, dried over NaSO₄, filtered, and concentrated. The residue was purified by column chromatography to give the title compound (910 mg).

### Step G: benzyl 7′-(1-amino-5-(tert-butoxy)-1,5-dioxopentan-2-yl)-6′-oxo-1′,6′,7′,8′-tetrahydro-2′H-spiro[piperidine-4,3′-pyrrolo[3,4-g]indole]-1-carboxylate

Benzyl 4-(((2-(1-amino-5-(tert-butoxy)-1,5-dioxopentan-2-yl)-5-bromo-1-oxoisoindolin-4-yl)amino)methyl)-3,6-dihydropyridine-1(2H)-carboxylate (1.30 g, 2.03 mmol), Bu₃SnH (2.36 g, 8.12 mmol) and AIBN (333 mg, 2.03 mmol) were added in toluene (40 mL) at 20° C. and then stir the mixture at 110° C. for 12 h. After the reaction completed, the mixture was quenched by addition saturated potassium fluoride solution and stirred for 1 h. The product was extracted with EA. The organic layers were dried over Na₂SO₄ and concentrated under reduced pressure. The residue was purified by column chromatography on silica gel to afford the title compound (500 mg).

¹H NMR (600 MHz, Methanol-d₄) δ 7.40-7.35 (m, 4H), 7.34-7.30 (m, 1H), 7.16 (d, J=7.6 Hz, 1H), 7.12 (d, J=7.6 Hz, 1H), 5.15 (s, 2H), 4.95 (dd, J=10.1, 4.5 Hz, 1H), 4.51 (d, J=17.2 Hz, 1H), 4.35 (d, J=17.2 Hz, 1H), 4.12 (dq, J=14.3, 2.7 Hz, 2H), 3.62-3.55 (m, 2H), 3.02 (brs, 2H), 2.31-2.17 (m, 4H), 1.86-1.65 (m, 4H), 1.38 (s, 9H).

### Step H: tert-butyl (S)-5-amino-5-oxo-4-(6′-oxo-1′,2′,6′,8′-tetrahydro-7′H-spiro[piperidine-4,3′-pyrrolo[3,4-g]indol]-7′-yl)pentanoate

To a 100 mL flask equipped with a magnetic stirring bar was added benzyl 7′-(1-amino-5-(tert-butoxy)-1,5-dioxopentan-2-yl)-6′-oxo-1′,6′,7′,8′-tetrahydro-2′H-spiro[piperidine-4,3′-pyrrolo[3,4-g]indole]-1-carboxylate (65 mg), THF (5 mL), and then 10% Pd/C (10 mg). The flask was evacuated and flushed three times with hydrogen, and the reaction mixture was stirred for 2 h under hydrogen atmosphere (balloon). Upon full consumption of the starting material by TLC monitoring, the reaction mixture was filtered through celite and washed through with additional MeOH. Solvent was removed and the residual tert-butyl (S)-5-amino-5-oxo-4-(6′-oxo-1′,2′,6′,8′-tetrahydro-7′H-spiro[piperidine-4,3′-pyrrolo[3,4-g]indol]-7′-yl)pentanoate was obtained (45 mg) as a white solid which was used directly in the next step.

### Compound B1: (S)-3-(1′-((1-(4-chlorophenyl)-5-(trifluoromethyl)-1H-pyrazol-4-yl) methyl)-6-oxo-6,8-dihydro-2H,7H-spiro[furo[2,3-e]isoindole-3,4′-piperidin]-7-yl) piperidine-2,6-dione

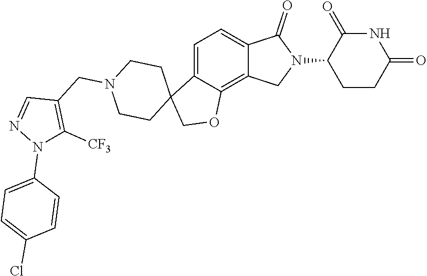 [SMILES: O=C1CC[C@H](N2Cc3c(ccc4c3OCC43CCN(Cc4cnn(-c5ccc(Cl)cc5)c4C(F)(F)F)CC3)C2=O)C(=O)N1]

To a suspension of (S)-3-(6-oxo-6,8-dihydro-2H,7H-spiro[furo[2,3-e]isoindole-3,4′-piperidin]-7-yl)piperidine-2,6-dione (Intermediate B2, 50.0 mg, 128 μmol, 1.0 eq) in MeOH (10.0 mL) was added Diisopropylethylamine (165 mg, 220 μL, 1.28 mmol, 10.0 eq) at room temperature. After 5-10 min, 1-(4-chlorophenyl)-5-(trifluoromethyl)-1H-pyrazole-4-carbaldehyde (Intermediate B12, 52.6 mg, 191 μmol, 1.5 eq) and Acetic acid (76.6 mg, 73.3 μL, 1.28 mmol, 10.0 eq) were added to above mixture at room temperature (pH ˜6). The reaction mixture was stirred at 25° C. for 4 h. Then Sodium cyanoborohydride (16.0 mg, 255 μmol, 2.0 eq) was added and the resulting reaction mixture was stirred at 25° C. for 1 h. After filtration, the filtrate was concentrated under reduced pressure. The crude product was purified by Prep-TLC (DCM/MeOH=10/1) to give impure product as a white solid. The impure product was further purified by Prep-HPLC with YMC-Actus Triart C18 (5 μm, 250×21 mm), and mobile phase of 5-95% ACN in water (0.1% FA) over 10 min and then hold at 100% ACN for 3 min, at a flow rate of 20 mL/min to give (S)-3-(1′-((1-(4-chlorophenyl)-5-(trifluoromethyl)-1H-pyrazol-4-yl)methyl)-6-oxo-6,8-dihydro-2H,7H-spiro[furo[2,3-e]isoindole-3,4′-piperidin]-7-yl)piperidine-2,6-dione (6.50 mg, yield 8%) as a white solid. as a white solid. LC-MS (ESI): mass calcd. for C₃₀H₂₇ClF₃N₅O₄, 613.17; m/z found, 614.2 [M+H]⁺.

¹H NMR (400 MHz, DMSO-d₆) δ 10.97 (s, 1H), 7.90 (s, 1H), 7.65 (d, J=7.8 Hz, 2H), 7.55 (d, J=7.8 Hz, 2H), 7.40 (d, J=7.8 Hz, 1H), 7.28 (d, J=7.8 Hz, 1H), 5.11-5.07 (m, 1H), 4.55 (s, 2H), 4.39 (d, J=17.0 Hz, 1H), 4.22 (d, J=17.0 Hz, 1H), 3.59 (s, 2H), 2.95-2.86 (m, 3H), 2.61-2.57 (m, 1H), 2.45-2.37 (m, 1H), 2.16-2.08 (m, 2H), 2.00-1.94 (m, 3H), 1.75-1.72 (m, 2H).

### Compound B2: (S)-3-(1′-((3-(4-chlorophenyl)isoxazol-5-yl)methyl)-6-oxo-6,8-dihydro-2H,7H-spiro[furo[2,3-e]isoindole-3,4′-piperidin]-7-yl)piperidine-2,6-dione

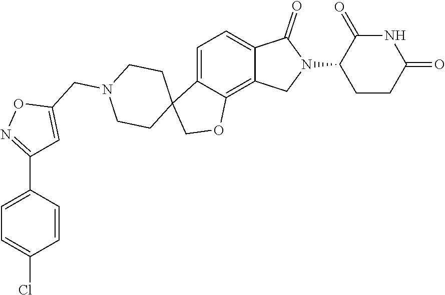 [SMILES: O=C1CC[C@H](N2Cc3c(ccc4c3OCC43CCN(Cc4cc(-c5ccc(Cl)cc5)no4)CC3)C2=O)C(=O)N1]

The title compound was prepared according to the procedure described in Compound B-1 by reductive amination between (S)-3-(6-oxo-6,8-dihydro-2H,7H-spiro[furo[2,3-e]isoindole-3,4′-piperidin]-7-yl)piperidine-2,6-dione (Intermediate B2) and 3-(4-chlorophenyl)isoxazole-5-carbaldehyde (Intermediate B13) as a white solid. LC-MS (ESI): mass calced for: C₂₉H₂₇ClN₄O₅, 546.17; m/z found, 547.3 [M+H]⁺. ¹H NMR (400 MHz, DMSO-d₆) δ 10.97 (s, 1H), 7.92 (d, J=8.4 Hz, 2H), 7.58 (d, J=8.4 Hz, 2H), 7.40 (d, J=7.2 Hz, 1H), 7.28 (d, J=7.4 Hz, 1H), 7.04 (s, 1H), 5.11-5.06 (m, 1H), 4.53 (s, 2H), 4.38 (d, J=17.2 Hz, 1H), 4.21 (d, J=17.2 Hz, 1H), 3.79 (s, 2H), 2.92-2.88 (m, 3H), 2.61-2.56 (m, 1H), 2.44-2.38 (m, 1H), 2.20 (t, J=12.2 Hz, 2H), 1.98 (d, J=7.6 Hz, 3H), 1.70 (t, J=10.6 Hz, 2H).

### Compound B3: (S)-3-(1′-((3-(4-chlorophenyl)isothiazol-5-yl)methyl)-6-oxo-6,8-dihydro-2H,7H-spiro[furo[2,3-e]isoindole-3,4′-piperidin]-7-yl)piperidine-2,6-dione

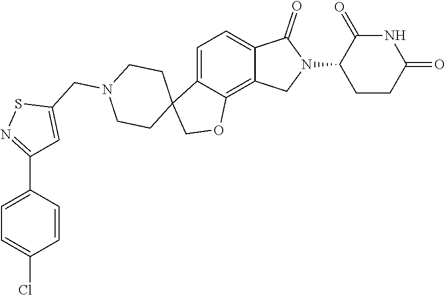 [SMILES: O=C1CC[C@H](N2Cc3c(ccc4c3OCC43CCN(Cc4cc(-c5ccc(Cl)cc5)ns4)CC3)C2=O)C(=O)N1]

The title compound was prepared according to the procedure described in Compound B-1 by reductive amination between (S)-3-(6-oxo-6,8-dihydro-2H,7H-spiro[furo[2,3-e]isoindole-3,4′-piperidin]-7-yl)piperidine-2,6-dione (Intermediate B2) and 3-(4-chlorophenyl)isothiazole-5-carbaldehyde (Intermediate B14) as a white solid. LC-MS (ESI): mass calcd. for C₂₉H₂₇ClN₄O₄S, 562.1; m/z found, 563.2 (M+H)⁺. ¹H NMR (400 MHz, DMSO-d₆) δ 10.97 (s, 1H), 8.03 (d, J=8.8 Hz, 2H), 7.85 (s, 1H), 7.55 (d, J=8.8 Hz, 2H), 7.45 (d, J=7.6 Hz, 1H), 7.28 (d, J=7.6 Hz, 1H), 5.11-5.06 (m, 1H), 4.58-4.50 (m, 2H), 4.38 (d, J=17.2 Hz, 1H), 4.22 (d, J=17.2 Hz, 1H), 3.96 (s, 2H), 2.98-2.85 (m, 3H), 2.61-2.56 (m, 1H), 2.43-2.39 (m, 1H), 2.22 (t, J=11.6 Hz, 2H), 1.96 (t, J=12.4 Hz, 3H), 1.73 (t, J=11.4 Hz, 2H).

### Compound B4: (S)-3-(1′-((3-chloro-1-(4-chlorophenyl)-1H-pyrazol-4-yl)methyl)-6-oxo-6,8-dihydro-2H,7H-spiro[furo[2,3-e]isoindole-3,4′-piperidin]-7-yl)piperidine-2,6-dione

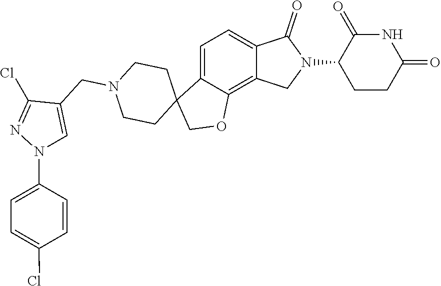 [SMILES: O=C1CC[C@H](N2Cc3c(ccc4c3OCC43CCN(Cc4cn(-c5ccc(Cl)cc5)nc4Cl)CC3)C2=O)C(=O)N1]

The title compound was prepared according to the procedure described in Compound B-1 by reductive amination between (S)-3-(6-oxo-6,8-dihydro-2H,7H-spiro[furo[2,3-e]isoindole-3,4′-piperidin]-7-yl)piperidine-2,6-dione (Intermediate B2) and 3-chloro-1-(4-chlorophenyl)-1H-pyrazole-4-carbaldehyde (Intermediate B15) as a white solid. LC-MS (ESI): mass calced for: C₂₉H₂₇Cl₂N₅O₄ 579.1; m/z found, 580.1 [M+H]⁺. ¹H NMR (400 MHz, DMSO-d₆) δ 10.97 (s, 1H), 8.61 (s, 1H), 7.86 (d, J=8.8 Hz, 2H), 7.57 (d, J=8.8 Hz, 2H), 7.37 (d, J=7.6 Hz, 1H), 7.27 (d, J=7.6 Hz, 1H), 5.10-5.05 (m, 1H), 4.60-4.45 (m, 2H), 4.38 (d, J=17.0 Hz, 1H), 4.21 (d, J=17.0 Hz, 1H), 3.45 (s, 2H), 2.90-2.87 (m, 3H), 2.60-2.56 (m, 1H), 2.46-2.36 (m, 1H), 2.09 (t, J=11.7 Hz, 2H), 1.98-1.89 (m, 3H), 1.70 (t, J=11.0 Hz, 2H).

### Compound B5: (S)-3-(1′-((1-(4-chlorophenyl)-3-(trifluoromethyl)-1H-pyrazol-4-yl)methyl)-6-oxo-6,8-dihydro-2H,7H-spiro[furo[2,3-e]isoindole-3,4′-piperidin]-7-yl)piperidine-2,6-dione

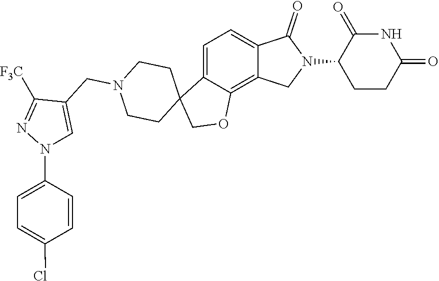 [SMILES: O=C1CC[C@H](N2Cc3c(ccc4c3OCC43CCN(Cc4cn(-c5ccc(Cl)cc5)nc4C(F)(F)F)CC3)C2=O)C(=O)N1]

The title compound was prepared according to the procedure described in Compound B-1 by reductive amination between (S)-3-(6-oxo-6,8-dihydro-2H,7H-spiro[furo[2,3-e]isoindole-3,4′-piperidin]-7-yl)piperidine-2,6-dione (Intermediate B2) and 1-(4-chlorophenyl)-3-(trifluoromethyl)-1H-pyrazole-4-carbaldehyde (Intermediate B16) as a white solid. LC-MS (ESI): mass calcd. for C₃₀H₂₇ClF₃N₅O₄, 613.2; m/z found, 614.2 (M+H)⁺.

¹H NMR (400 MHz, DMSO-d₆) δ 10.97 (s, 1H), 8.70 (s, 1H), 7.94 (d, J=8.8 Hz, 2H), 7.62 (d, J=8.8 Hz, 2H), 7.36 (d, J=7.6 Hz, 1H), 7.28 (d, J=7.6 Hz, 1H), 5.10-5.06 (m, 1H), 4.54 (s, 2H), 4.38 (d, J=17.0 Hz, 1H), 4.21 (d, J=17.0 Hz, 1H), 3.55 (s, 2H), 2.98-2.83 (m, 3H), 2.60-2.56 (m, 1H), 2.44-2.35 (m, 1H), 2.15-2.04 (m, 2H), 2.01-1.86 (m, 3H), 1.77-1.65 (m, 2H).

### Compound B6: (S)-3-(6-oxo-1′-((1-((tetrahydro-2H-pyran-4-yl)methyl)-1H-pyrazol-4-yl)methyl)-6,8-dihydro-2H,7H-spiro[furo[2,3-e]isoindole-3,4′-piperidin]-7-yl)piperidine-2,6-dione

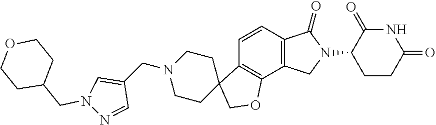 [SMILES: O=C1CC[C@H](N2Cc3c(ccc4c3OCC43CCN(Cc4cnn(CC5CCOCC5)c4)CC3)C2=O)C(=O)N1]

The title compound was prepared according to the procedure described in Compound B-1 by reductive amination between (S)-3-(6-oxo-6,8-dihydro-2H,7H-spiro[furo[2,3-e]isoindole-3,4′-piperidin]-7-yl)piperidine-2,6-dione (Intermediate B2) and 1-((tetrahydro-2H-pyran-4-yl)methyl)-1H-pyrazole-4-carbaldehyde (Intermediate B17) as a white solid. LC-MS (ESI): mass calcd. for C₂₉H₃₅N₅O₅, 533.0; m/z found, 534.2 [M+H]⁺. ¹H NMR (400 MHz, DMSO-d₆) δ 10.97 (s, 1H), 8.16 (s, 1H), 7.61 (s, 1H), 7.40-7.34 (m, 2H), 7.26 (d, J=7.6 Hz, 1H), 5.09-5.05 (m, 1H), 4.49 (s, 2H), 4.37 (d, J=17.2 Hz, 1H), 4.21 (d, J=17.2 Hz, 1H), 3.96 (d, J=7.2 Hz, 2H), 3.83-3.80 (m, 2H), 3.41 (s, 2H), 3.24 (dd, J=11.6, 9.6 Hz, 2H), 2.94-2.81 (m, 3H), 2.64-2.59 (m, 1H), 2.46-2.34 (m, 1H), 2.04-1.89 (m, 5H), 1.68 (t, J=10.8 Hz, 2H), 1.36 (d, J=10.8 Hz, 2H), 1.27-1.14 (m, 3H).

### Compound B7: (S)-3-(1′-((2-methyl-2H-indazol-6-yl)methyl-d2)-6-oxo-6,8-dihydro-2H,7H-spiro[furo[2,3-e]isoindole-3,4′-piperidin]-7-yl)piperidine-2,6-dione

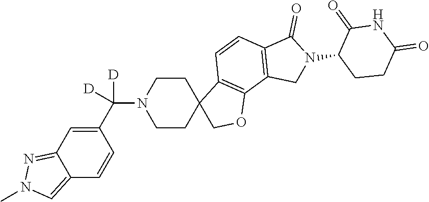 [SMILES: [2H]C([2H])(c1ccc2cn(C)nc2c1)N1CCC2(CC1)COc1c2ccc2c1CN([C@H]1CCC(=O)NC1=O)C2=O]

To a solution of (S)-3-(6-oxo-6,8-dihydro-2H,7H-spiro[furo[2,3-e]isoindole-3,4′-piperidin]-7-yl)piperidine-2,6-dione (Intermediate B2, 13.0 mg, 36.7 μmol, 1.0 eq) in DMF (1.0 mL) was added N-ethyl-N-isopropylpropan-2-amine (14.2 mg, 110 μmol, 3.0 eq) at 0° C. and the mixture was stirred at 0° C. for 15 min. Then 6-(bromomethyl-d₂)-2-methyl-2H-indazole (Intermediate B18, 10.0 mg, 44.0 μmol, 1.2 eq) was added to above mixture and the reaction mixture was stirred at 25° C. for 30 min. After evaporation, the residue was purified with Prep-HPLC with YMC-Actus Triart C18 (5 um, 20×250 mm), and mobile phase of 5-95% ACN in water (0.1% HCOOH) over 20 min and then hold at 100% ACN for 2 min, at a flow rate of 25 mL/min to give (S)-3-(1′-((2-methyl-2H-indazol-6-yl)methyl-d2)-6-oxo-6,8-dihydro-2H,7H-spiro[furo[2,3-e]isoindole-3,4′-piperidin]-7-yl) piperidine-2,6-dione formate (8.0 mg, yield 43%) as a white solid. LC-MS (ESI): mass calcd. for C₂₈H₂₇D₂N₅O₄, 501.58; m/z found, 502.2 [M+H]⁺. ¹H NMR (400 MHz, DMSO-d₆) δ 10.97 (s, 1H), 8.27 (s, 1H), 8.15 (s, 1H), 7.64 (dd, J=8.6, 0.8 Hz, 1H), 7.47 (d, J=0.8 Hz, 1H), 7.42 (d, J=7.6 Hz, 1H), 7.26 (d, J=7.6 Hz, 1H), 7.05 (dd, J=8.6, 1.2 Hz, 1H), 5.10-5.05 (m, 1H), 4.57-4.50 (m, 2H), 4.37 (d, J=17.2 Hz, 1H), 4.21 (d, J=17.2 Hz, 1H), 4.14 (s, 3H), 2.97-2.80 (m, 3H), 2.60-2.56 (m, 1H), 2.45-2.33 (m, 1H), 2.14-2.00 (m, 2H), 2.00-1.87 (m, 3H), 1.68 (t, J=11.2 Hz, 2H).

### Compound B8: (S)-3-(1′-((1-methyl-1H-indazol-6-yl)methyl-d2)-6-oxo-6,8-dihydro-2H,7H-spiro[furo[2,3-e]isoindole-3,4′-piperidin]-7-yl)piperidine-2,6-dione

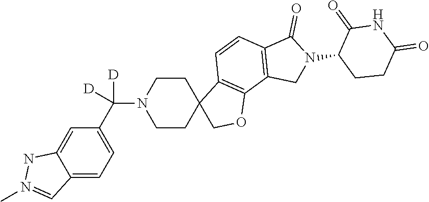

The title compound was prepared according to the procedure described in Compound B-7 by displacement between (S)-3-(6-oxo-6,8-dihydro-2H,7H-spiro[furo[2,3-e]isoindole-3,4′-piperidin]-7-yl)piperidine-2,6-dione (Intermediate B2) and 6-(bromomethyl-d2)-1-methyl-1H-indazole (Intermediate B19) as a white solid. LC-MS (ESI): mass calcd. for C₂₈H₂₇D₂N₅O₄, 501.58; m/z found, 502.2 [M+H]⁺. ¹H NMR (400 MHz, DMSO-d₆) δ 10.97 (s, 1H), 8.14 (s, 1H), 8.00 (d, J=0.8 Hz, 1H), 7.70 (d, J=8.4 Hz, 1H), 7.54 (s, 1H), 7.42 (d, J=7.6 Hz, 1H), 7.27 (d, J=7.6 Hz, 1H), 7.16 (dd, J=8.4, 1.2 Hz, 1H), 5.10-5.05 (m, 1H), 4.58-4.49 (m, 2H), 4.38 (d, J=17.2 Hz, 1H), 4.21 (d, J=17.2 Hz, 1H), 4.04 (s, 3H), 2.98-2.81 (m, 3H), 2.60-2.56 (m, 1H), 2.45-2.32 (m, 1H), 2.10 (t, J=11.6 Hz, 2H), 1.98-1.91 (m, 3H), 1.69 (t, J=11.0 Hz, 2H).

### Compound B9: (S)-3-(1′-((3-(1-(methyl-d3)-1H-pyrazol-4-yl)phenyl)methyl-d2)-6-oxo-6,8-dihydro-2H,7H-spiro[furo[2,3-e]isoindole-3,4′-piperidin]-7-yl)piperidine-2,6-dione

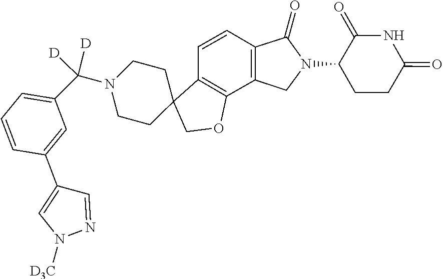 [SMILES: [2H]C([2H])(c1cccc(-c2cnn(C([2H])([2H])[2H])c2)c1)N1CCC2(CC1)COc1c2ccc2c1CN([C@H]1CCC(=O)NC1=O)C2=O]

The title compound was prepared according to the procedure described in Compound B-7 by displacement between (S)-3-(6-oxo-6,8-dihydro-2H,7H-spiro[furo[2,3-e]isoindole-3,4′-piperidin]-7-yl)piperidine-2,6-dione (Intermediate B2) and 4-(3-(bromomethyl-d2)phenyl)-1-(methyl-d3)-1H-pyrazole (Intermediate B20) as a white solid. LC-MS (ESI): mass calcd. for C₃₀H₂₆D₅N₅O₄, 530.27; m/z found, 531.3 [M+H]⁺. ¹H NMR (400 MHz, DMSO-d₆) δ 10.97 (s, 1H), 8.15 (s, 1H), 8.13 (s, 1H), 7.85 (s, 1H), 7.49 (s, 1H), 7.43 (dd, J=12.0, 7.6 Hz, 2H), 7.31 (t, J=7.6 Hz, 1H), 7.27 (d, J=7.6 Hz, 1H), 7.16 (d, J=7.6 Hz, 1H), 5.10-5.06 (m, 1H), 4.53 (s, 2H), 4.38 (d, J=17.2 Hz, 1H), 4.21 (d, J=17.2 Hz, 1H), 2.95-2.81 (m, 3H), 2.61-2.56 (m, 1H), 2.43-2.36 (m, 1H), 2.06 (t, J=11.6 Hz, 2H), 2.01-1.87 (m, 3H), 1.69 (t, J=11.2 Hz, 2H).

### Compound B10: (S)-3-(1′-((3-(1-(difluoromethyl)-1H-pyrazol-4-yl) phenyl) methyl-d2)-6-oxo-6,8-dihydro-2H,7H-spiro[furo[2,3-e]isoindole-3,4′-piperidin]-7-yl) piperidine-2,6-dione

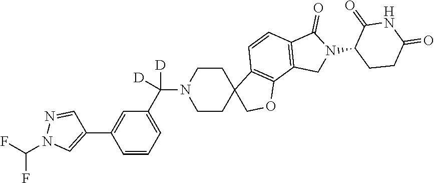 [SMILES: [2H]C([2H])(c1cccc(-c2cnn(C(F)F)c2)c1)N1CCC2(CC1)COc1c2ccc2c1CN([C@H]1CCC(=O)NC1=O)C2=O]

The title compound was prepared according to the procedure described in Compound B-7 by displacement between (S)-3-(6-oxo-6,8-dihydro-2H,7H-spiro[furo[2,3-e]isoindole-3,4′-piperidin]-7-yl)piperidine-2,6-dione (Intermediate B2) and 4-(3-(bromomethyl-d2)phenyl)-1-(difluoromethyl)-1H-pyrazole (Intermediate B21) as a white solid. LC-MS (ESI): mass calcd. for C₃₀H₂₇D₂F₂N₅O₄, 563.23; m/z found, 564.2 [M+H]⁺. ¹H NMR (400 MHz, DMSO-d₆) δ 10.97 (s, 1H), 8.73 (s, 1H), 8.28 (s, 2H), 7.83 (t, J=59.2 Hz, 1H), 7.64 (s, 1H), 7.59 (d, J=7.8 Hz, 1H), 7.39 (dd, J=17.4, 7.6 Hz, 2H), 7.27 (d, J=7.6 Hz, 2H), 5.10-5.05 (m, 1H), 4.56-4.50 (m, 2H), 4.38 (d, J=17.21 Hz, 1H), 4.21 (d, J=17.2 Hz, 1H), 2.96-2.88 (m, 1H), 2.85 (d, J=10.4 Hz, 2H), 2.62-2.55 (m, 1H), 2.47-2.36 (m, 1H), 2.07 (t, J=11.8 Hz, 2H), 2.01-1.89 (m, 3H), 1.69 (t, J=11.4 Hz, 2H).

### Compound B11: (S)-3-(1′-((3-(1-methyl-1H-pyrazol-4-yl)phenyl)methyl-d2)-6-oxo-6,8-dihydro-2H,7H-spiro[furo[2,3-e]isoindole-3,4′-piperidin]-7-yl)piperidine-2,6-dione

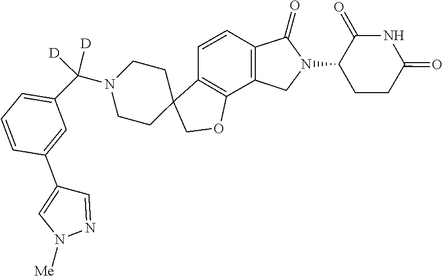 [SMILES: [2H]C([2H])(c1cccc(-c2cnn(C)c2)c1)N1CCC2(CC1)COc1c2ccc2c1CN([C@H]1CCC(=O)NC1=O)C2=O]

The title compound was prepared according to the procedure described in Compound B-7 by displacement between (S)-3-(6-oxo-6,8-dihydro-2H,7H-spiro[furo[2,3-e]isoindole-3,4′-piperidin]-7-yl)piperidine-2,6-dione (Intermediate B2) and 4-(3-(bromomethyl-d2)phenyl)-1-methyl-1H-pyrazole (Intermediate B22) as a white solid. LC-MS (ESI): mass calcd. for C₃₀H₂₉D₂N₅O₄, 527.25; m/z found, 528.2 [M+H]⁺. ¹H NMR (400 MHz, DMSO-d₆) δ 10.96 (s, 1H), 8.13 (s, 1H), 7.85 (s, 1H), 7.51 (s, 1H), 7.46 (d, J=7.8 Hz, 1H), 7.41 (d, J=7.8 Hz, 1H), 7.33 (s, 1H), 7.28 (d, J=7.6 Hz, 1H), 7.18 (d, J=7.8 Hz, 1H), 5.10-5.06 (m, 1H), 4.54 (s, 2H), 4.38 (d, J=17.2 Hz, 1H), 4.21 (d, J=17.2 Hz, 1H), 3.87 (s, 3H), 2.97-2.84 (m, 3H), 2.61-2.56 (m, 1H), 2.45-2.32 (m, 1H), 2.14-2.11 (m, 2H), 1.99-1.92 (m, 3H), 1.71 (t, J=11.6 Hz, 2H).

### Compound B12: (S)-3-(1′-(3-(1-(methyl-d₃)-1H-pyrazol-4-yl)benzyl)-6-oxo-6,8-dihydro-2H,7H-spiro[furo[2,3-e]isoindole-3,4′-piperidin]-7-yl)piperidine-2,6-dione

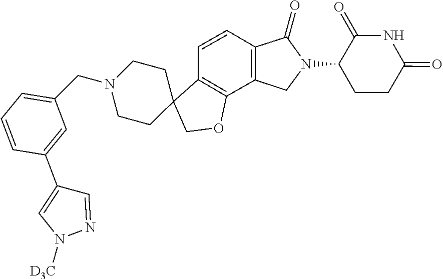 [SMILES: [2H]C([2H])([2H])n1cc(-c2cccc(CN3CCC4(CC3)COc3c4ccc4c3CN([C@H]3CCC(=O)NC3=O)C4=O)c2)cn1]

The title compound was prepared according to the procedure described in Compound B-1 by reductive amination between (S)-3-(6-oxo-6,8-dihydro-2H,7H-spiro[furo[2,3-e]isoindole-3,4′-piperidin]-7-yl)piperidine-2,6-dione (Intermediate B2) and 3-(1-(methyl-d₃)-1H-pyrazol-4-yl)benzaldehyde (Intermediate B23) as a white solid. LC-MS (ESI): mass calcd. for C₃₀H₂₈D₃N₅O₄, 528.26; m/z found, 529.3 [M+H]⁺. ¹H NMR (400 MHz, DMSO-d₆) δ 10.96 (s, 1H), 8.14 (s, 1H), 8.13 (s, 1H), 7.85 (s, 1H), 7.50 (s, 1H), 7.43 (dd, J=13.2, 7.6 Hz, 2H), 7.32 (t, J=7.6 Hz, 1H), 7.27 (d, J=7.6 Hz, 1H), 7.16 (d, J=7.6 Hz, 1H), 5.10-5.05 (m, 1H), 4.54 (s, 2H), 4.38 (d, J=17.2 Hz, 1H), 4.21 (d, J=17.2 Hz, 1H), 3.53 (s, 2H), 2.94-2.83 (m, 3H), 2.60-2.56 (m, 1H), 2.43-2.36 (m, 1H), 2.08 (t, J=11.8 Hz, 2H), 2.08-1.90 (m, 3H), 1.69 (t, J=11.4 Hz, 2H).

### Compound B13: 3-(1′-(3-(1-methyl-1H-pyrazol-4-yl)benzyl)-6-oxo-6,8-dihydro-2H,7H spiro[furo[2,3-e]isoindole-3,4′-piperidin]-7-yl-2,2-d2)piperidine-2,6-dione

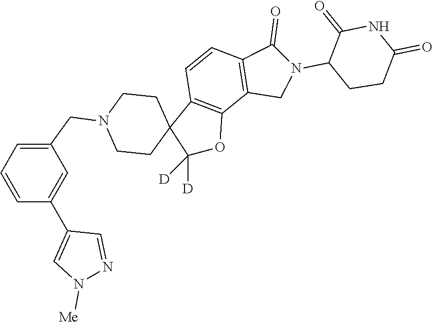 [SMILES: [2H]C1([2H])Oc2c(ccc3c2CN(C2CCC(=O)NC2=O)C3=O)C12CCN(Cc1cccc(-c3cnn(C)c3)c1)CC2]

The title compound was prepared according to the procedure described in Compound B-1 by reductive amination between (S)-3-(6-oxo-6,8-dihydro-2H,7H-spiro[furo[2,3-e]isoindole-3,4′-piperidin]-7-yl-2,2-d2)piperidine-2,6-dione (Intermediate B3) and 3-(1-methyl-1H-pyrazol-4-yl)benzaldehyde (Intermediate B27) as a white solid. LC-MS (ESI): mass calcd. for C₃₀H₂₉D₂N₅O₄, 527.62; m/z found, 528.4 [M+H]⁺. ¹H NMR (400 MHz, DMSO-d₆) δ 10.97 (s, 1H), 8.30 (s, 1H), 8.13 (s, 1H), 7.85 (s, 1H), 7.50 (s, 1H), 7.43 (dd, J=11.6, 7.6 Hz, 2H), 7.30 (dd, J=13.4, 5.8 Hz, 1H), 7.27 (d, J=7.6 Hz, 1H), 7.16 (d, J=7.6 Hz, 1H), 5.10-5.05 (m, 1H), 4.38 (d, J=17.2 Hz, 1H), 4.21 (d, J=17.2 Hz, 1H), 3.87 (s, 3H), 3.52 (s, 2H), 2.98-2.79 (m, 3H), 2.560-2.56 (m, 1H), 2.44-2.32 (m, 1H), 2.06 (t, J=12.0 Hz, 2H), 2.00-1.87 (m, 3H), 1.69 (t, J=10.8 Hz, 2H).

### Compound B14: (R)-3-(1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione

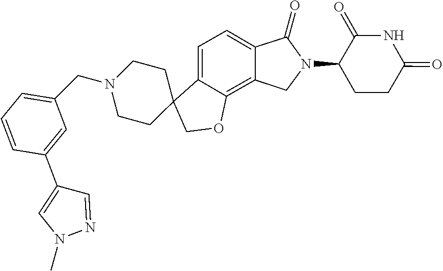 [SMILES: Cn1cc(-c2cccc(CN3CCC4(CC3)COc3c4ccc4c3CN([C@@H]3CCC(=O)NC3=O)C4=O)c2)cn1]

To a suspension of (S)-3-(6-oxo-6,8-dihydro-2H,7H-spiro[furo[2,3-e]isoindole-3,4′-piperidin]-7-yl) piperidine-2,6-dione (Intermediate B2, 320 mg, 817 μmol, 1.0 eq) in DMF (20.0 mL) was added triethylamine (826 mg, 1.14 mL, 8.17 mmol, 10.0 eq) at room temperature. After 5-10 min, 3-(1-methyl-1H-pyrazol-4-yl) benzaldehyde (Intermediate B27, 304 mg, 1.63 mmol, 2.0 eq) and Acetic acid (981 mg, 939 μL, 16.3 mmol, 20.0 eq) were added to above mixture at room temperature (pH ˜6). The reaction mixture was stirred at 25° C. for 2 h, and then sodium triacetoxyborohydride (346 mg, 1.63 mmol, 2.0 eq) was added and the resulting reaction mixture was stirred at 25° C. for 2 h. The resulting mixture was quenched with saturated aqueous NH₄Cl solution (30 mL) and extracted with EtOAc (50 mL×3). The combined organic phases were washed with brine (50 mL×4), dried over anhydrous Na₂SO₄, filtered and concentrated under reduced pressure. The residue was purified by flash column chromatography on silica gel (DCM/MeOH=50/1 to 15/1 v/v) to provide (S)-3-(1′-(3-(1-methyl-1H-pyrazol-4-yl) benzyl)-6-oxo-6,8-dihydro-2H,7H-spiro[furo[2,3-e]isoindole-3,4′-piperidin]-7-yl) piperidine-2,6-dione (290 mg, yield 86%) (80% e.e.) as a yellow solid. The product was slurred with a mixed solvent of MeOH (10 mL) and MeCN (10 mL), filtered, and the filtrate was concentrated under reduced pressure to give (S)-3-(1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione (A170) (130 mg, 95% e.e.). The cake (˜100 mg, R/S mixture) was separated by SFC Prep-HPLC with IMADZU PREP SOLUTION SFC with ChiralPak 1H (ChiralPak 1H, 250×21.2 mm I.D., 5 μm), and mobile phase of A for CO₂ and B for ETOH+0.1% NH₃H₂O 5-99% over 5 h, at a flow rate of 40 mL/min to give(S)-3-(1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione (30.0 mg, yield 20.0%, 100% e.e) as a white solid. And (R)-3-(1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione (12.7 mg, yield 8.5%, 96.5% e.e.) as a white solid. LC-MS (ESI): mass calcd. for C₃₀H₃₁N₅O₄, 525.24; m/z found, 526.3 [M+H]⁺. ¹H NMR (400 MHz, DMSO-d₆) δ 10.96 (s, 1H), 8.13 (s, 1H), 7.85 (s, 1H), 7.49 (s, 1H), 7.46-7.40 (m, 2H), 7.33-7.26 (m, 2H), 7.16 (d, J=7.6 Hz, 1H), 5.10-5.06 (m, 1H), 4.54 (s, 2H), 4.38 (d, J=17.2 Hz, 1H), 4.21 (d, J=17.2 Hz, 1H), 3.87 (s, 3H), 3.52 (s, 2H), 2.90-2.82 (m, 3H), 2.58 (d, J=17.8 Hz, 1H), 2.43-2.40 (m, 1H), 2.06-2.02 (m, 2H), 1.97-1.93 (m, 3H), 1.71-1.66 (m, 2H).

### Compound B15: (R)-3-(1′-(3-(1-(oxetan-3-yl)-1H-pyrazol-4-yl)benzyl)-6-oxo-6,8-dihydro-2H,7H-spiro[furo[2,3-e]isoindole-3,4′-piperidin]-7-yl)piperidine-2,6-dione

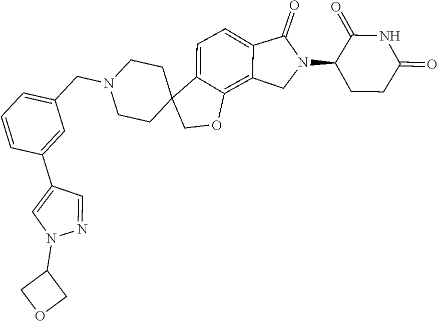 [SMILES: O=C1CC[C@@H](N2Cc3c(ccc4c3OCC43CCN(Cc4cccc(-c5cnn(C6COC6)c5)c4)CC3)C2=O)C(=O)N1]

To a stirred mixture of (S)-3-(6-oxo-6,8-dihydro-2H,7H-spiro[furo[2,3-e]isoindole-3,4′-piperidin]-7-yl)piperidine-2,6-dione (Intermediate B2, 850 mg, 2.17 mmol, 1.0 eq) in MeOH (5.0 mL) was added DIPEA (336.2 mg, 2.6 mmol, 1.2 eq) at room temperature and the mixture was stirred for 15 min. 3-(1-(oxetan-3-yl)-1H-pyrazol-4-yl)benzaldehyde (Intermediate B28, 743 mg, 3.25 mmol, 1.5 eq) and AcOH (260.4 mg, 4.34 mmol, 2.0 eq) were added to above mixture and the reaction mixture was stirred at room temperature for 3 h. Then NaBH₃CN (341 mg, 5.42 mmol, 2.5 eq) was added to above mixture and the reaction mixture was stirred at room temperature for 1 h. After evaporation, the residue was purified by flash column chromatography on silica gel (DCM/MeOH=10/1 v/v) to get a crude product. The crude product was further purified by Prep-HPLC with YMC-Actus Triart 18C (5 μm, 20×250 mm), and mobile phase of 5-99% ACN in water (0.1% FA) over 10 min and then hold at 100% ACN for 2 min, at a flow rate of 25 mL/min to (S)-3-(1′-(3-(1-(oxetan-3-yl)-1H-pyrazol-4-yl)benzyl)-6-oxo-6,8-dihydro-2H,7H-spiro[furo[2,3-e]isoindole-3,4′-piperidin]-7-yl)piperidine-2,6-dione (650 mg, ˜90% e.e., yield 53%). The racemate product was further separated by SFC Prep-HPLC with Waters Thar 80 preparative SFC (ChiralPak 1H, 150×21.2 mm I.D., 5 μm), and mobile phase of A for CO₂ and B for 0.1% 7 mol/L NH₃ in EtOH over 4 h, at a flow rate of 40 mL/min to get (S)-3-(1′-(3-(1-(oxetan-3-yl)-1H-pyrazol-4-yl)benzyl)-6-oxo-6,8-dihydro-2H,7H-spiro[furo[2,3-e]isoindole-3,4′-piperidin]-7-yl)piperidine-2,6-dione (350 mg, yield 54%) as a white solid and 60 mg of racemate mixture (S/R=1/1). The racemate mixture (60 mg) was second separated by SFC by SFC Prep-HPLC with Waters Thar 80 preparative SFC (ChiralPak 1H, 150×21.2 mm I.D., 5 μm), and mobile phase of A for CO₂ and B for 0.1% 7 mol/L NH₃ in EtOH over 4 h, at a flow rate of 40 mL/min to afford (R)-3-(1′-(3-(1-(oxetan-3-yl)-1H-pyrazol-4-yl)benzyl)-6-oxo-6,8-dihydro-2H,7H-spiro[furo[2,3-e]isoindole-3,4′-piperidin]-7-yl)piperidine-2,6-dione (9.6 mg, yield 16%) as a white solid. LC-MS (ESI): mass calced for: C₃₂H₃₃N₅O₅ 567.65; m/z found, 568.2 [M+H]⁺. ¹H NMR (400 MHz, DMSO-d₆) δ 10.97 (s, 1H), 8.38 (s, 1H), 8.02 (s, 1H), 7.54 (s, 1H), 7.49 (d, J=8.0 Hz, 1H), 7.41 (d, J=8.4 Hz, 1H), 7.33 (t, J=7.6 Hz, 1H), 7.27 (d, J=8.0 Hz, 1H), 7.19 (d, J=7.6 Hz, 1H), 5.67-5.50 (m, 1H), 5.11-5.07 (m, 1H), 4.94 (d, J=7.4 Hz, 4H), 4.54 (s, 2H), 4.38 (d, J=17.2 Hz, 1H), 4.21 (d, J=17.2 Hz, 1H), 3.52 (s, 2H), 2.97-2.76 (m, 3H), 2.59 (d, J=16.6 Hz, 1H), 2.44-2.39 (m, 1H), 2.09-2.03 (m, 2H), 1.98-1.90 (m, 3H), 1.69 (t, J=11.4 Hz, 2H).

### Compound B16: (S)-3-(5-fluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione

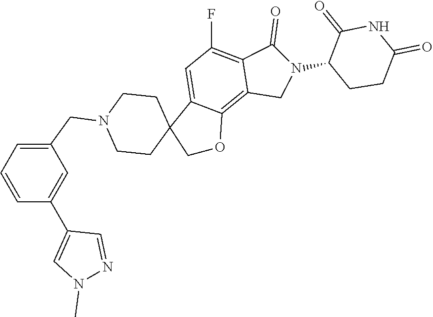 [SMILES: Cn1cc(-c2cccc(CN3CCC4(CC3)COc3c4cc(F)c4c3CN([C@H]3CCC(=O)NC3=O)C4=O)c2)cn1]

### Step A: tert-butyl (S)-5-amino-4-(5-fluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)-5-oxopentanoate

A solution of tert-butyl (S)-5-amino-4-(5-fluoro-6-oxo-6,8-dihydro-2H,7H-spiro[furo[2,3-e]isoindole-3,4′-piperidin]-7-yl)-5-oxopentanoate (Intermediate B4, 60 mg, 1.0 eq.) and 3-(1-methyl-1H-pyrazol-4-yl)benzaldehyde (23 mg, 10 eq.) in DMAc (3 mL) was added acetic acid (0.01 mL). The resulting reaction mixture was stirred at ambient temperature for 15 min. Then sodium triacetoxyborohydride (57 mg, 2 eq.) was added. The resulting reaction mixture was stirred at room temperature for 12 h. The reaction mixture was then diluted with water (100 mL) and extracted with DCM/MeOH=10:1 (x3). The organic phases were combined and dried over anhydrous Na₂SO₄. The solvent was removed under reduced pressure. The resulting crude material was purified by flash chromatography to afford the title product as a white solid (60 mg). LC-MS (m/z): [M+H]⁺=618.40.

### Step B: (S)-3-(5-fluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione

To the suspension of tert-butyl (S)-5-amino-4-(5-fluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)-5-oxopentanoate (40 mg, 1 eq) in ACN (1 mL) was added Benzenesulfonic acid (40 mg, 4 eq). The resulting suspension was stirred at 90° C. for 5 h. The reaction mixture was allowed to cool to room temperature and added TFA, then purified by prep-HPLC quickly to afford a white solid as the final product (18 mg). LC-MS (m/z): [M+H]⁺=544.40.

### Compound B17: (S)-3-(1′-benzyl-5-chloro-6-oxo-6,8-dihydro-2H,7H-spiro[furo[2,3-e]isoindole-3,4′-piperidin]-7-yl)piperidine-2,6-dione

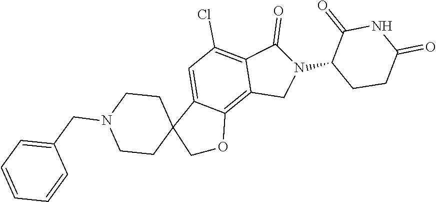 [SMILES: O=C1CC[C@H](N2Cc3c4c(cc(Cl)c3C2=O)C2(CCN(Cc3ccccc3)CC2)CO4)C(=O)N1]

The title compound was prepared according to the procedure described in Compound B-1 by reductive amination between (S)-3-(5-chloro-6-oxo-6,8-dihydro-2H,7H-spiro[furo[2,3-e]isoindole-3,4′-piperidin]-7-yl)piperidine-2,6-dione (Intermediate B5) and benzaldehyde as a white solid. LC-MS (ESI): mass calcd. for C₂₆H₂₆ClN₃O₄, 479.2; m/z found, 480.0 [M+H]⁺.

¹H NMR (400 MHz, DMSO) δ 10.98 (s, 1H), 8.26 (s, 1H), 7.64-7.08 (m, 6H), 5.04 (s, 1H), 4.56 (s, 2H), 4.34 (d, J=17.2 Hz, 1H), 4.18 (d, J=17.0 Hz, 1H), 3.50 (s, 2H), 2.90 (s, 1H), 2.79 (s, 2H), 2.60 (s, 1H), 2.40-2.37 (m, 1H), 2.07-1.88 (m, 5H), 1.68 (s, 2H).

### Compound B18: (S)-3-(1′-((1H-indazol-7-yl)methyl)-5-chloro-6-oxo-6,8-dihydro-2H,7H-spiro[furo[2,3-e]isoindole-3,4′-piperidin]-7-yl)piperidine-2,6-dione

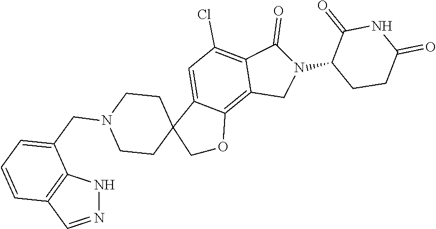 [SMILES: O=C1CC[C@H](N2Cc3c4c(cc(Cl)c3C2=O)C2(CCN(Cc3cccc5cn[nH]c35)CC2)CO4)C(=O)N1]

The title compound was prepared according to the procedure described in Compound B-1 by reductive amination between (S)-3-(5-chloro-6-oxo-6,8-dihydro-2H,7H-spiro[furo[2,3-e]isoindole-3,4′-piperidin]-7-yl)piperidine-2,6-dione (Intermediate B5) and 1H-indazole-7-carbaldehyde as a white solid. LC-MS (ESI): mass calced for C₂₇H₂₆ClN₅O₄ 519; m/z found, 520.2 [M+H]⁺. ¹H NMR (400 MHz, DMSO-d₆) δ 12.88 (s, 1H), 10.98 (s, 1H), 8.13 (s, 1H), 8.10 (s, 1H), 7.67 (s, 1H), 7.46 (s, 1H), 7.25 (s, 1H), 7.08 (s, 1H), 5.08-5.03 (m, 1H), 4.59 (s, 2H), 4.35 (d, J=17.4 Hz, 1H), 4.18 (d, J=17.4 Hz, 1H), 3.82 (s, 2H), 2.93-2.85 (m, 3H), 2.62-2.56 (m, 1H), 2.47-2.43 (m, 1H), 2.18-1.91 (m, 5H), 1.68 (s, 2H).

### Compound B19: (S)-7-(2,6-dioxopiperidin-3-yl)-1′-(3-(1-methyl-1H-pyrazol-4-yl)benzyl)-6-oxo-7,8-dihydro-2H,6H-spiro[furo[2,3-e]isoindole-3,4′-piperidine]-5-carbonitrile

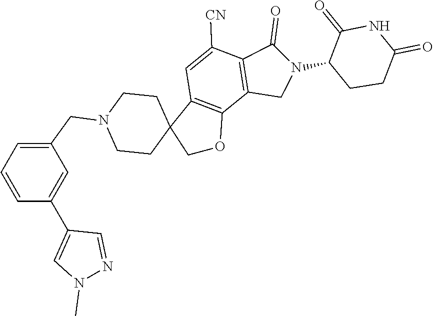 [SMILES: Cn1cc(-c2cccc(CN3CCC4(CC3)COc3c4cc(C#N)c4c3CN([C@H]3CCC(=O)NC3=O)C4=O)c2)cn1]

The title compound was prepared according to the procedure described in Compound B-1 by reductive amination between (S)-7-(2,6-dioxopiperidin-3-yl)-6-oxo-7,8-dihydro-2H,6H-spiro[furo[2,3-e]isoindole-3,4′-piperidine]-5-carbonitrile (Intermediate B6) and 3-(1-methyl-1H-pyrazol-4-yl)benzaldehyde (Intermediate B27) as a white solid. LC-MS (ESI): mass calced for: C₃₁H₃₃N₅O₄ 550.23; m/z found, 551.2 [M+H]⁺. ¹H NMR (400 MHz, DMSO-d₆) δ 11.02 (s, 1H), 8.28 (s, 1H), 8.14 (s, 1H), 7.99 (s, 1H), 7.85 (s, 1H), 7.50 (s, 1H), 7.45 (d, J=7.6 Hz, 1H), 7.32 (t, J=7.6 Hz, 1H), 7.17 (d, J=7.6 Hz, 1H), 5.11-5.07 (m, 1H), 4.68 (s, 2H), 4.45 (d, J=17.8 Hz, 1H), 4.29 (d, J=17.8 Hz, 1H), 3.88 (s, 3H), 3.53 (s, 2H), 2.94-2.83 (m, 3H), 2.62-2.57 (m, 1H), 2.45-2.29 (m, 1H), 2.09-1.96 (m, 5H), 1.71 (t, J=11.2 Hz, 2H).

### Compound B20: (S)-3-(5-methyl-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione

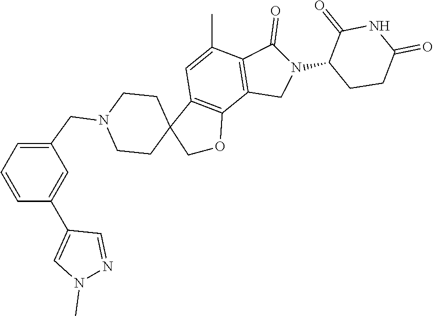 [SMILES: Cc1cc2c(c3c1C(=O)N([C@H]1CCC(=O)NC1=O)C3)OCC21CCN(Cc2cccc(-c3cnn(C)c3)c2)CC1]

The title compound was prepared according to the procedure described in Compound B-1 by reductive amination between (S)-3-(5-methyl-6-oxo-6,8-dihydro-2H,7H-spiro[furo[2,3-e]isoindole-3,4′-piperidin]-7-yl)piperidine-2,6-dione (Intermediate B7) and 3-(1-methyl-1H-pyrazol-4-yl)benzaldehyde (Intermediate B27) as a brown solid. LC-MS (ESI): mass calced for: C₃₁H₃₃N₅O₄ 539.3; m/z found, 540.3 [M+H]⁺. ¹H NMR (400 MHz, DMSO-d₆) δ 10.96 (s, 1H), 8.33 (s, 1H), 8.14 (s, 1H), 7.85 (s, 1H), 7.50 (s, 1H), 7.45 (d, J=7.8 Hz, 1H), 7.32 (t, J=7.6 Hz, 1H), 7.16 (d, J=6.0 Hz, 2H), 5.05-5.01 (m, 1H), 4.49 (s, 2H), 4.30 (d, J=17.2 Hz, 1H), 4.14 (d, J=17.2 Hz, 1H), 3.87 (s, 3H), 3.52 (s, 2H), 2.96-2.89 (m, 1H), 2.86-2.82 (m, 2H), 2.63-2.57 (m, 1H), 2.55 (s, 3H), 2.39-2.36 (m, 1H), 2.09-2.22 (m, 2H), 1.97-1.92 (m, 3H), 1.67 (t, J=10.4 Hz, 2H).

### Compound B21: 3-(3′-methyl-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione

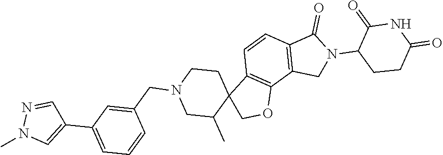 [SMILES: CC1CN(Cc2cccc(-c3cnn(C)c3)c2)CCC12COc1c2ccc2c1CN(C1CCC(=O)NC1=O)C2=O]

The title compound was prepared according to the procedure described in Compound B-1 by reductive amination between 3-(3′-methyl-6-oxo-6,8-dihydro-2H,7H-spiro[furo[2,3-e]isoindole-3,4′-piperidin]-7-yl)piperidine-2,6-dione (Intermediate B28) and 3-(1-methyl-1H-pyrazol-4-yl)benzaldehyde (Intermediate B27) as a white solid. LC-MS (ESI): mass calcd. for C₃₁H₃₃N₅O₄, 539.64; m/z found, 540.4 [M+H]⁺. ¹H NMR (400 MHz, DMSO-d₆) δ 10.97 (s, 1H), 8.13 (d, J=4.4 Hz, 1H), 7.84 (d, J=4.8 Hz, 1H), 7.47 (dd, J=22.8, 8.3 Hz, 2H), 7.39-7.26 (m, 3H), 7.16 (d, J=7.0 Hz, 1H), 5.10-5.05 (m, 1H), 4.69-4.66 (m, 1H), 4.41-4.22 (m, 3H), 3.87 (s, 3H), 3.49 (s, 2H), 2.95-2.74 (m, 3H), 2.61-2.57 (m, 1H), 2.43-2.39 (m, 1H), 2.11-1.87 (m, 4H), 1.78-1.74 (m, 2H), 0.75-0.52 (m, 3H).

### Compound B22: 3-(1′-benzyl-3′-hydroxy-6-oxo-6,8-dihydro-2H,7H-spiro[furo[2,3-e]isoindole-3,4′-piperidin]-7-yl)piperidine-2,6-dione

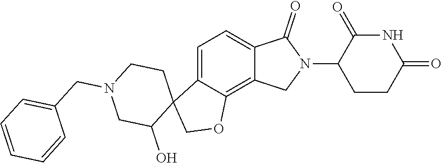 [SMILES: O=C1CCC(N2Cc3c(ccc4c3OCC43CCN(Cc4ccccc4)CC3O)C2=O)C(=O)N1]

The title compound was prepared according to the procedure described in Compound B-1 by reductive amination between 3-(3′-hydroxy-6-oxo-6,8-dihydro-2H,7H-spiro[furo[2,3-e]isoindole-3,4′-piperidin]-7-yl)piperidine-2,6-dione (Intermediate B29) and benzaldehyde as a white solid. LC-MS (ESI): mass calcd. for C₂₆H₂₇N₃O₅, 461.52; m/z found, 462.1 [M+H]⁺.

¹H NMR (400 MHz, DMSO-d₆) δ 10.95 (s, 1H), 7.38-7.23 (m, 7H), 5.08-5.04 (m, 1H), 4.95 (s, 1H), 4.77-4.75 (m, 1H), 4.45-4.31 (m, 2H), 4.19 (d, J=17.0 Hz, 1H), 3.81 (dd, J=10.4, 4.4 Hz, 1H), 3.56 (d, J=13.2 Hz, 1H), 3.44 (d, J=13.2 Hz, 1H), 2.95-2.71 (m, 3H), 2.63-2.54 (m, 1H), 2.43-2.39 (m, 1H), 2.02-1.88 (m, 3H), 1.77-1.72 (m, 2H).

### Compound B23: 3-(1′-benzyl-3′-fluoro-6-oxo-6,8-dihydro-2H,7H-spiro[furo[2,3-e]isoindole-3,4′-piperidin]-7-yl)piperidine-2,6-dione

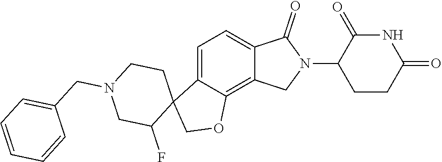 [SMILES: O=C1CCC(N2Cc3c(ccc4c3OCC43CCN(Cc4ccccc4)CC3F)C2=O)C(=O)N1]

To a solution of 3-(1′-benzyl-3′-hydroxy-6-oxo-6,8-dihydro-2H,7H-spiro[furo[2,3-e]isoindole-3,4′-piperidin]-7-yl)piperidine-2,6-dione (Example B-22, 20.0 mg, 43.3 μmol, 1.0 eq) in DCM (40.0 mL) was added DAST (140 mg, 115 μL, 867 μmol, 20.0 eq) and the reaction mixture was stirred at room temperature overnight. The mixture was washed with saturated aqueous NaHCO₃ solution (30 mL×3) and brine (30 mL×3), dried over anhydrous sodium sulfate, filtered and concentrated under reduced pressure. The residue was purified with Prep-HPLC with YMC-Actus Triart C18 (5 um, 20×250 mm), and mobile phase of 5-95% ACN in water (0.1% HCOOH) over 20 min and then hold at 100% ACN for 2 min, at a flow rate of 25 mL/min to afford 3-(1′-benzyl-3′-fluoro-6-oxo-6,8-dihydro-2H,7H-spiro[furo[2,3-e]isoindole-3,4′-piperidin]-7-yl)piperidine-2,6-dione (10.0 mg, yield 50%) as a white solid. LC-MS (ESI): mass calcd. for C₂₆H₂₆FN₃O₄, 463.51; m/z found, 464.2 [M+H]⁺. ¹H NMR (400 MHz, DMSO-d₆) δ 10.99 (s, 1H), 7.61-7.41 (m, 7H), 5.13-5.09 (m, 1H), 5.02-4.53 (m, 4H), 4.47-4.36 (m, 2H), 4.30-4.24 (m, 2H), 3.92 (s, 1H), 3.30 (s, 2H), 2.99-2.85 (m, 1H), 2.62-2.58 (m, 2H), 2.46-2.39 (m, 1H), 2.23-2.19 (m, 1H), 2.00-1.97 (m, 1H).

### Compound B24: 3-(1′-((1H-indazol-7-yl)methyl)-3′-hydroxy-6-oxo-6,8-dihydro-2H,7H-spiro[furo[2,3-e]isoindole-3,4′-piperidin]-7-yl)piperidine-2,6-dione

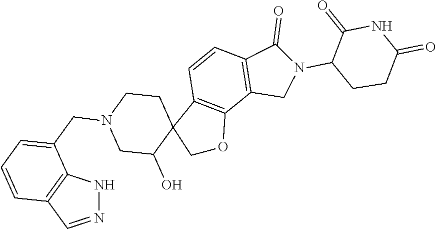 [SMILES: O=C1CCC(N2Cc3c(ccc4c3OCC43CCN(Cc4cccc5cn[nH]c45)CC3O)C2=O)C(=O)N1]

The title compound was prepared according to the procedure described in Compound B-1 by reductive amination between 3-(3′-hydroxy-6-oxo-6,8-dihydro-2H,7H-spiro[furo[2,3-e]isoindole-3,4′-piperidin]-7-yl)piperidine-2,6-dione (Intermediate B29) and 1H-indazole-7-carbaldehyde as a white solid. LC-MS (ESI): mass calcd. for C₂₇H₂₇N₅O₅, 501.54; m/z found, 502.3 [M+H]⁺. ¹H NMR (400 MHz, DMSO-d₆) δ 12.97 (s, 1H), 10.98 (s, 1H), 8.15-8.10 (m, 1H), 7.70-7.60 (m, 4H), 7.10-7.06 (m, 1H), 5.11-4.17 (m, 6H), 4.07-3.48 (m, 3H), 2.95-2.57 (m, 4H), 2.46-2.40 (m, 1H), 2.24-2.18 (m, 1H), 2.04-1.95 (m, 2H), 1.74-1.57 (m, 2H).

### Compound B25: 3-(1′-benzyl-3′,3′-difluoro-6-oxo-6,8-dihydro-2H,7H-spiro[furo[2,3-e]isoindole-3,4′-piperidin]-7-yl)piperidine-2,6-dione

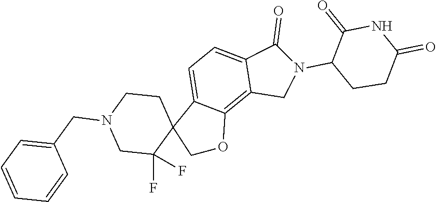 [SMILES: O=C1CCC(N2Cc3c(ccc4c3OCC43CCN(Cc4ccccc4)CC3(F)F)C2=O)C(=O)N1]

The title compound was prepared according to the procedure described in Compound B-1 by reductive amination between 3-(3′,3′-difluoro-6-oxo-6,8-dihydro-2H,7H-spiro[furo[2,3-e]isoindole-3,4′-piperidin]-7-yl)piperidine-2,6-dione (Intermediate B10) and benzaldehyde as a white solid. LC-MS (ESI): mass calcd. for C₂₆H₂₅F₂N₃O₄, 481.50; m/z found, 482.6 [M+H]⁺. ¹H NMR (400 MHz, DMSO-d₆) δ 10.99 (d, J=3.6 Hz, 1H), 8.17 (s, 1H), 7.50 (d, J=7.4 Hz, 1H), 7.41-7.23 (m, 6H), 5.12-5.08 (m, 1H), 4.87-4.85 (m, 1H), 4.55 (t, J=8.0 Hz, 1H), 4.41 (t, J=16.4 Hz, 1H), 4.24 (t, J=16.4 Hz, 1H), 3.65 (s, 2H), 3.09 (s, 1H), 2.95-2.81 (m, 2H), 2.69-2.54 (m, 2H), 2.46-2.36 (m, 1H), 2.28 (t, J=11.2 Hz, 1H), 2.14 (t, J=11.6 Hz, 1H), 2.04-1.88 (m, 2H).

### Compound B26: 3-(3′,3′-difluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione

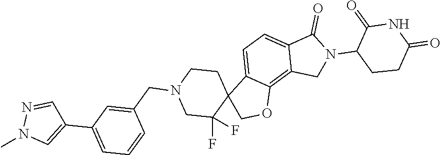 [SMILES: Cn1cc(-c2cccc(CN3CCC4(COc5c4ccc4c5CN(C5CCC(=O)NC5=O)C4=O)C(F)(F)C3)c2)cn1]

The title compound was prepared according to the procedure described in Compound B-1 by reductive amination between 3-(3′,3′-difluoro-6-oxo-6,8-dihydro-2H,7H-spiro[furo[2,3-e]isoindole-3,4′-piperidin]-7-yl)piperidine-2,6-dione (Intermediate B10) and 3-(1-methyl-1H-pyrazol-4-yl)benzaldehyde (Intermediate B27) as a white solid. LC-MS (ESI): mass calcd. for C₃₀H₂₉F₂N₅O₄, 561.59; m/z found, 562.2 [M+H]⁺. ¹H NMR (400 MHz, DMSO-d₆) δ 10.99 (s, 1H), 8.14 (s, 1H), 7.85 (s, 1H), 7.52-7.47 (m, 3H), 7.37-7.32 (m, 2H), 7.18 (d, J=7.6 Hz, 1H), 5.11-5.08 (m, 1H), 4.90-4.86 (m, 1H), 4.60-4.52 (m, 1H), 4.42 (t, J=16.4 Hz, 1H), 4.25 (t, J=16.4 Hz, 1H), 3.87 (s, 3H), 3.72-3.61 (m, 2H), 3.12 (s, 1H), 2.96-2.82 (m, 2H), 2.70-2.56 (m, 2H), 2.47-2.37 (m, 1H), 2.39-2.30 (m, 1H), 2.16 (t, J=11.6 Hz, 1H), 2.04-1.88 (m, 2H).

### Compound B27: 4-(4-((7-((S)-2,6-dioxopiperidin-3-yl)-3′,3′-difluoro-6-oxo-7,8-dihydro-2H,6H-spiro[furo[2,3-e]isoindole-3,4′-piperidin]-1′-yl)methyl)-1H-pyrazol-1-yl)benzonitrile

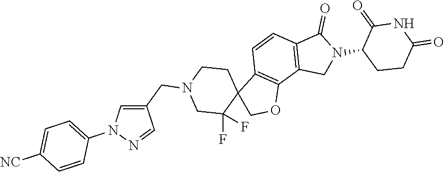 [SMILES: N#Cc1ccc(-n2cc(CN3CCC4(COc5c4ccc4c5CN([C@H]5CCC(=O)NC5=O)C4=O)C(F)(F)C3)cn2)cc1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 4-(4-formyl-1H-pyrazol-1-yl)benzonitrile (Intermediate B24) as a white solid. LC-MS (ESI): mass calcd. for C₃₀H₂₆F₂N₆O₄, 572.20; m/z found, 573.3 [M+H]⁺. ¹H NMR (400 MHz, DMSO-d₆) δ 10.97 (s, 1H), 8.63 (s, 1H), 8.07 (d, J=8.8 Hz, 2H), 7.98 (d, J=8.8 Hz, 2H), 7.81 (s, 1H), 7.47 (d, J=7.6 Hz, 1H), 7.32 (d, J=7.6 Hz, 1H), 5.12-5.08 (m, 1H), 4.85 (d, J=9.4 Hz, 1H), 4.54 (d, J=9.6 Hz, 1H), 4.43 (d, J=17.2 Hz, 1H), 4.22 (d, J=17.2 Hz, 1H), 3.64 (s, 2H), 3.20-3.16 (m, 1H), 2.97-2.83 (m, 2H), 2.60-2.56 (m, 2H), 2.43-2.40 (m, 1H), 2.28 (d, J=13.2 Hz, 1H), 2.13 (t, J=11.2 Hz, 1H), 1.98-1.93 (m, 2H). ¹⁹F NMR (376 MHz, DMSO-d₆) δ −107.67 (s), −108.31 (s).

### Compound B28: (3S)-3-(3′,3′-difluoro-6-oxo-1′-((2-oxoindolin-5-yl)methyl)-6,8-dihydro-2H,7H-spiro[furo[2,3-e]isoindole-3,4′-piperidin]-7-yl)piperidine-2,6-dione

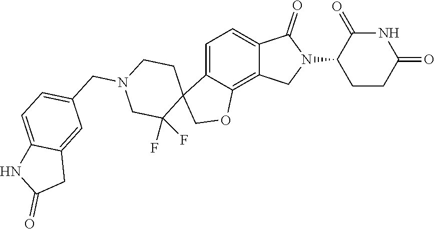 [SMILES: O=C1CC[C@H](N2Cc3c(ccc4c3OCC43CCN(Cc4ccc5c(c4)CC(=O)N5)CC3(F)F)C2=O)C(=O)N1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 2-oxoindoline-5-carbaldehyde (Intermediate B25) as a white solid. LC-MS (ESI): mass calcd. for C₂₈H₂₆F₂N₄05, 536.19; m/z found, 537.4 [M+H]⁺. ¹H NMR (400 MHz, DMSO-d₆) δ 10.99 (s, 1H), 10.37 (s, 1H), 8.49 (s, 1H), 7.49 (d, J=7.6 Hz, 1H), 7.32 (d, J=7.6 Hz, 1H), 7.18 (s, 1H), 7.12 (d, J=8.0 Hz, 1H), 6.79 (d, J=8.0 Hz, 1H), 5.11-5.07 (m, 1H), 4.88-4.83 (m, 1H), 4.56-4.54 (m, 1H), 4.41 (t, J=17.2 Hz, 1H), 4.24 (t, J=17.2 Hz, 1H), 3.56 (s, 2H), 3.48 (s, 2H), 3.09 (s, 1H), 2.91-2.83 (m, 2H), 2.62-2.56 (m, 2H), 2.50-2.48 (m, 1H), 2.26-2.23 (m, 1H), 2.10-2.06 (m, 1H), 2.00-1.96 (m, 2H).

### Compound B29: (3S)-3-(1′-((1-((2-chlorophenyl)sulfonyl)-1H-pyrazol-4-yl)methyl)-3′,3′-difluoro-6-oxo-6,8-dihydro-2H,7H-spiro[furo[2,3-e]isoindole-3,4′-piperidin]-7-yl)piperidine-2,6-dione

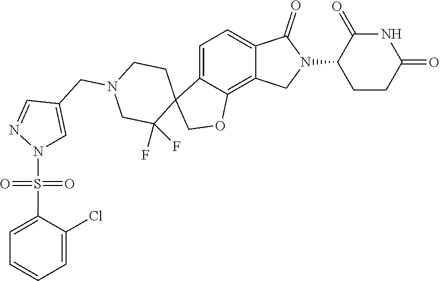 [SMILES: O=C1CC[C@H](N2Cc3c(ccc4c3OCC43CCN(Cc4cnn(S(=O)(=O)c5ccccc5Cl)c4)CC3(F)F)C2=O)C(=O)N1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1-((2-chlorophenyl)sulfonyl)-1H-pyrazole-4-carbaldehyde (Intermediate B26) as a white solid. LC-MS (ESI): mass calcd. for C₂₉H₂₆ClN₅O₆S, 645.12; m/z found, 646.3 [M+H]⁺. ¹H NMR (400 MHz, DMSO-d₆) δ 10.99 (d, J=4.0 Hz, 1H), 8.55 (s, 1H), 8.25-8.19 (m, 1H), 7.90 (s, 1H), 7.82 (dd, J=12.2, 4.8 Hz, 1H), 7.75 (d, J=7.2 Hz, 1H), 7.70 (t, J=7.6 Hz, 1H), 7.48 (d, J=7.4 Hz, 1H), 7.32 (d, J=7.6 Hz, 1H), 5.14-5.04 (m, 1H), 4.85-4.76 (m, 1H), 4.53 (s, 1H), 4.42 (t, J=17.0 Hz, 1H), 4.25 (t, J=17.0 Hz, 1H), 3.65 (s, 2H), 3.13 (s, 2H), 2.91-2.88 (m, 2H), 2.60 (d, J=15.4 Hz, 1H), 2.44-2.40 (m, 1H), 2.28-2.25 (m, 1H), 2.13-2.11 (m, 1H), 2.01-1.86 (m, 2H). ¹⁹F NMR (376 MHz, DMSO) δ −107.88 (s), −108.52 (s).

### Compound B30: (3S)-3-(1′-((1-(4-chlorophenyl)-1H-pyrazol-4-yl)methyl)-3′,3′-difluoro-6-oxo-6,8-dihydro-2H,7H-spiro[furo[2,3-e]isoindole-3,4′-piperidin]-7-yl)piperidine-2,6-dione

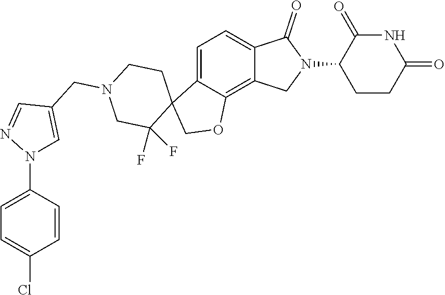 [SMILES: O=C1CC[C@H](N2Cc3c(ccc4c3OCC43CCN(Cc4cnn(-c5ccc(Cl)cc5)c4)CC3(F)F)C2=O)C(=O)N1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1-(4-chlorophenyl)-1H-pyrazole-4-carbaldehyde as a white solid. LC-MS (ESI): mass calcd. for C₂₉H₂₆ClF₂N₅O₄, 581.16; m/z found, 582.4 [M+H]⁺. ¹H NMR (400 MHz, DMSO-d₆) δ 10.98 (s, 1H), 8.49 (s, 1H), 7.89 (d, J=8.8 Hz, 2H), 7.73 (s, 1H), 7.57 (d, J=8.8 Hz, 2H), 7.48-7.46 (m, 1H), 7.33 (d, J=7.8 Hz, 1H), 5.13-5.08 (m, 1H), 4.85 (t, J=9.4 Hz, 1H), 4.55 (d, J=9.0 Hz, 1H), 4.42 (t, J=17.2 Hz, 1H), 4.24 (t, J=17.2 Hz, 1H), 3.63 (s, 2H), 3.18 (s, 1H), 2.98-2.82 (m, 2H), 2.70-2.54 (m, 2H), 2.45-2.40 (m, 1H), 2.34-2.29 (m, 1H), 2.12 (t, J=15.2 Hz, 1H), 2.01-1.95 (m, 2H).

### Compound B31: (3S)-3-(3′,3′-difluoro-6-oxo-1′-((3-oxoisoindolin-5-yl)methyl)-6,8-dihydro-2H,7H-spiro[furo[2,3-e]isoindole-3,4′-piperidin]-7-yl)piperidine-2,6-dione

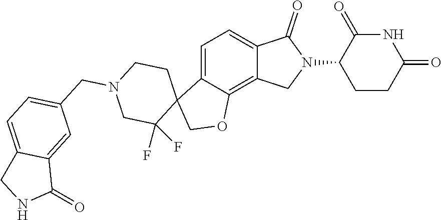 [SMILES: O=C1CC[C@H](N2Cc3c(ccc4c3OCC43CCN(Cc4ccc5c(c4)C(=O)NC5)CC3(F)F)C2=O)C(=O)N1]

The title compound was prepared according to the procedure described in Compound B1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 3-oxoisoindoline-5-carbaldehyde (Intermediate B29) as a white solid. LC-MS (ESI): mass calced for: C₂₈H₂₆F₂N₄O₅ 536.54; m/z found, 537.4 [M+H]⁺. ¹H NMR (400 MHz, DMSO-d₆) δ 10.98 (d, J=3.8 Hz, 1H), 8.56 (s, 1H), 7.66 (s, 1H), 7.57 (s, 2H), 7.52 (d, J=7.6 Hz, 1H), 7.32 (d, J=7.6 Hz, 1H), 5.18-5.03 (m, 1H), 4.89-4.85 (m, 1H), 4.57-4.55 (m, 1H), 4.43 (t, J=16.8 Hz, 1H), 4.37 (s, 2H), 4.24 (t, J=16.8 Hz, 1H), 3.77 (s, 2H), 3.13 (s, 1H), 2.98-2.80 (m, 2H), 2.66-2.57 (m, 2H), 2.45-2.36 (m, 1H), 2.35-2.26 (m, 1H), 2.18 (s, 1H), 1.99-1.90 (m, 2H). ¹⁹F NMR (376 MHz, DMSO-d₆) δ −108.01 (s), −108.65 (s).

### Compound B32: (3S)-3-(3′,3′-difluoro-1′-(3-(1-(oxetan-3-yl)-1H-pyrazol-4-yl)benzyl)-6-oxo-6,8-dihydro-2H,7H-spiro[furo[2,3-e]isoindole-3,4′-piperidin]-7-yl)piperidine-2,6-dione

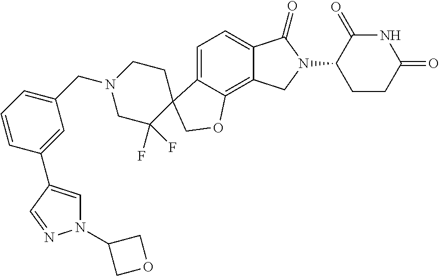 [SMILES: O=C1CC[C@H](N2Cc3c(ccc4c3OCC43CCN(Cc4cccc(-c5cnn(C6COC6)c5)c4)CC3(F)F)C2=O)C(=O)N1]

The title compound was prepared according to the procedure described in Compound B1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 3-(1-(oxetan-3-yl)-1H-pyrazol-4-yl)benzaldehyde (Intermediate B28) as a white solid. LC-MS (ESI): mass calced for: C₃₂H₃₁F₂N₅O₅ 603.63; m/z found, 604.3 [M+H]⁺. ¹H NMR (400 MHz, DMSO-d₆) δ 8.37 (s, 1H), 8.03 (s, 1H), 7.53 (dd, J=15.0, 7.2 Hz, 3H), 7.40-7.31 (m, 2H), 7.20 (d, J=7.6 Hz, 1H), 5.64-5.56 (m, 1H), 5.11-5.07 (m, 1H), 4.98-4.90 (m, 4H), 4.87 (dd, J=10.0, 5.4 Hz, 1H), 4.56 (t, J=8.2 Hz, 1H), 4.41 (t, J=16.6 Hz, 1H), 4.24 (t, J=16.6 Hz, 1H), 3.67 (s, 2H), 3.14 (s, 1H), 2.88 (d, J=12.4 Hz, 2H), 2.65-2.57 (m, 2H), 2.46-2.37 (m, 1H), 2.36-2.25 (m, 1H), 2.19-2.13 (m, 1H), 2.00-1.93 (m, 2H). ¹⁹FNMR (376 MHz, DMSO-d₆) δ −107.93 (s), −108.58 (s).

### Compound B33: (3S)-3-(1′-((1H-indazol-6-yl)methyl)-3′,3′-difluoro-3-oxo-1,3,7,8-tetrahydro-2H-spiro[cyclopenta[e]isoindole-6,4′-piperidin]-2-yl)piperidine-2,6-dione

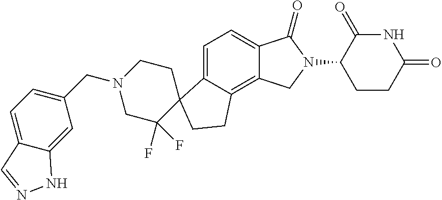 [SMILES: O=C1CC[C@H](N2Cc3c(ccc4c3CCC43CCN(Cc4ccc5cn[nH]c5c4)CC3(F)F)C2=O)C(=O)N1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1H-indazole-6-carbaldehyde as a white solid. LC-MS (ESI) mass calcd. for C₂₈H₂₇F₂N₅O₃, 519.21; m/z found, 520.4 [M+H]⁺. ¹H NMR (400 MHz, DMSO-d₆) δ 13.09 (s, 1H), 10.97 (s, 1H), 8.07 (s, 1H), 7.76 (d, J=8.6 Hz, 1H), 7.57-7.47 (m, 2H), 7.34 (d, J=7.6 Hz, 1H), 7.14 (d, J=8.4 Hz, 1H), 5.11-5.07 (m, 1H), 4.89 (d, J=9.8 Hz, 1H), 4.60-4.59 (m, 1H), 4.43 (d, J=17.0 Hz, 1H), 4.23 (d, J=17.2 Hz, 1H), 3.96 (s, 2H), 3.06-2.84 (m, 3H), 2.63-2.53 (m, 2H), 2.43-2.36 (m, 2H), 2.33 (s, 1H), 2.00-1.97 (m, 2H).

### Compound B34: (3S)-3-(3′,3′-difluoro-1′-((1-methyl-1H-indazol-6-yl)methyl)-3-oxo-1,3,7,8-tetrahydro-2H-spiro[cyclopenta[e]isoindole-6,4′-piperidin]-2-yl)piperidine-2,6-dione

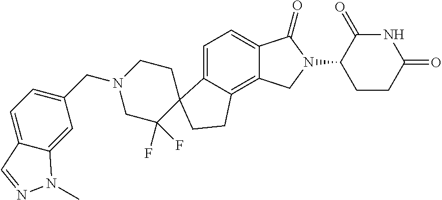 [SMILES: Cn1ncc2ccc(CN3CCC4(CCc5c4ccc4c5CN([C@H]5CCC(=O)NC5=O)C4=O)C(F)(F)C3)cc21]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1-methyl-1H-indazole-6-carbaldehyde (Intermediate B30a) as a white solid. LC-MS (ESI) mass calcd. for C₂₈H₂₇F₂N₅O₄, 535.20; m/z found, 536.2 [M+H]⁺. ¹H NMR (400 MHz, DMSO-d₆) δ 10.98 (s, 1H), 8.02 (s, 1H), 7.74 (d, J=8.4 Hz, 1H), 7.57 (s, 1H), 7.51 (d, J=7.8 Hz, 1H), 7.33 (d, J=7.8 Hz, 1H), 7.17 (d, J=8.4 Hz, 1H), 5.14-5.04 (m, 1H), 4.89 (dd, J=10.0, 5.2 Hz, 1H), 4.58-4.56 (m, 1H), 4.50-4.34 (m, 1H), 4.34-4.18 (m, 1H), 4.04 (s, 3H), 3.84 (s, 2H), 3.22-3.18 (m, 1H), 2.95-2.90 (m, 2H), 2.78-2.73 (m, 1H), 2.62-2.56 (m, 1H), 2.46-2.38 (m, 1H), 2.33-2.18 (m, 2H), 1.99-1.97 (m, 2H).

### Compound B35: (3S)-3-(3′,3′-difluoro-1′-((2-methyl-2H-indazol-6-yl)methyl)-6-oxo-6,8-dihydro-2H,7H-spiro[furo[2,3-e]isoindole-3,4′-piperidin]-7-yl)piperidine-2,6-dione

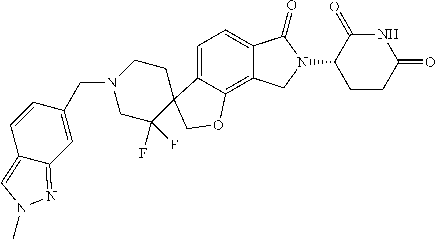 [SMILES: Cn1cc2ccc(CN3CCC4(COc5c4ccc4c5CN([C@H]5CCC(=O)NC5=O)C4=O)C(F)(F)C3)cc2n1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 2-methyl-2H-indazole-6-carbaldehyde (Intermediate B30b) as a white solid. LC-MS (ESI): mass calced for: C₂₈H₂₇F₂N₅O₄ 535.20; m/z found, 536.2 [M+H]⁺. ¹H NMR (400 MHz, DMSO-d₆) δ 10.97 (s, 1H), 8.29 (s, 1H), 7.67 (d, J=8.4 Hz, 1H), 7.51 (d, J=10.4 Hz, 2H), 7.32 (d, J=7.6 Hz, 1H), 7.05 (d, J=8.4 Hz, 1H), 5.11-5.07 (m, 1H), 4.86 (d, J=9.8 Hz, 1H), 4.56 (s, 1H), 4.41 (t, J=16.8 Hz, 1H), 4.24 (t, J=16.8 Hz, 1H), 4.15 (s, 3H), 3.71 (d, J=5.4 Hz, 2H), 3.11 (s, 1H), 2.90-2.86 (m, 2H), 2.61-2.56 (m, 2H), 2.44-2.37 (m, 1H), 2.28 (d, J=13.4 Hz, 1H), 2.19 (d, J=12.2 Hz, 1H), 2.00-1.937 (m, 2H). ¹⁹F NMR (376 MHz, DMSO-d₆) δ −107.93 (s), −108.58 (s).

### Compound B36: (3S)-3-(3′,3′-difluoro-1′-((2-methyl-2H-indazol-7-yl)methyl)-6-oxo-6,8-dihydro-2H,7H-spiro[furo[2,3-e]isoindole-3,4′-piperidin]-7-yl)piperidine-2,6-dione

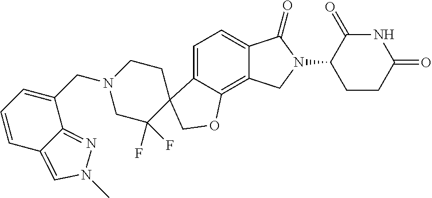 [SMILES: Cn1cc2cccc(CN3CCC4(COc5c4ccc4c5CN([C@H]5CCC(=O)NC5=O)C4=O)C(F)(F)C3)c2n1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 2-methyl-2H-indazole-7-carbaldehyde (Intermediate B31b) a white solid. LC-MS (ESI): mass calced for: C₂₈H₂₇F₂N₅O₄ 535.55; m/z found, 536.3 [M+H]⁺. ¹H NMR (400 MHz, DMSO-d₆) δ 10.97 (s, 1H), 8.34 (s, 1H), 7.61 (d, J=8.2 Hz, 1H), 7.51 (d, J=7.6 Hz, 1H), 7.32 (d, J=7.6 Hz, 1H), 7.25 (d, J=6.8 Hz, 1H), 7.09-6.99 (m, 1H), 5.16-5.03 (m, 1H), 4.87 (dd, J=9.8, 5.6 Hz, 1H), 4.56 (d, J=8.0 Hz, 1H), 4.41 (t, J=16.8 Hz, 1H), 4.26 (t, J=16.8 Hz, 1H), 4.18 (s, 3H), 4.02 (s, 2H), 3.23 (s, 1H), 2.98-2.87 (m, 2H), 2.77-2.58 (m, 2H), 2.46-2.18 (m, 3H), 2.01-1.89 (m, 2H). ¹⁹F NMR (376 MHz, DMSO) δ −107.95 (s), −108.59 (s).

### Compound B37: (3S)-3-(3′,3′-difluoro-1′-((2-methyl-2H-indazol-5-yl)methyl)-6-oxo-6,8-dihydro-2H,7H-spiro[furo[2,3-e]isoindole-3,4′-piperidin]-7-yl)piperidine-2,6-dione

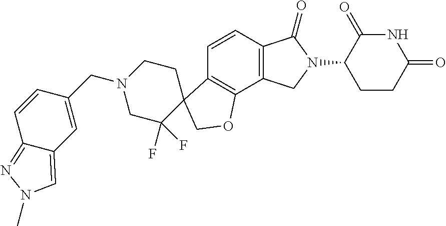 [SMILES: Cn1cc2cc(CN3CCC4(COc5c4ccc4c5CN([C@H]5CCC(=O)NC5=O)C4=O)C(F)(F)C3)ccc2n1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 2-methyl-2H-indazole-5-carbaldehyde (Intermediate B32b) as a white solid. LC-MS (ESI): mass calcd. for C₂₈H₂₇F₂N₅O₄, 535.20; m/z found, 536.2 (M+H)⁺. ¹H NMR (400 MHz, DMSO-d₆) δ 10.97 (s, 1H), 8.29 (s, 1H), 7.57 (d, J=10.0 Hz, 2H), 7.50 (d, J=7.2 Hz, 1H), 7.32 (d, J=7.6 Hz, 1H), 7.24 (d, J=8.4 Hz, 1H), 5.11-5.07 (m, 1H), 4.87-4.84 (m, 1H), 4.57-4.55 (m, 1H), 4.41 (t, J=16.6 Hz, 1H), 4.24 (t, J=16.6 Hz, 1H), 4.15 (s, 3H), 3.68 (s, 2H), 3.16-3.07 (m, 1H), 2.94-2.87 (m, 2H), 2.71-2.55 (m, 2H), 2.45-2.37 (m, 1H), 2.33-2.26 (m, 1H), 2.21-2.10 (m, 1H), 2.04-1.91 (m, 2H).

### Compound B38: (3S)-3-(3′,3′-difluoro-1′-((1-methyl-1H-indazol-5-yl) methyl)-6-oxo-6,8-dihydro-2H,7H-spiro[furo[2,3-e]isoindole-3,4′-piperidin]-7-yl) piperidine-2,6-dione

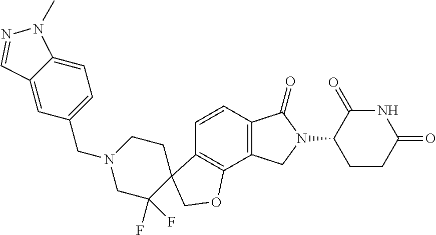 [SMILES: Cn1ncc2cc(CN3CCC4(COc5c4ccc4c5CN([C@H]5CCC(=O)NC5=O)C4=O)C(F)(F)C3)ccc21]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1-methyl-1H-indazole-5-carbaldehyde (Intermediate B32a) as a white solid. LC-MS (ESI): mass calcd. for C₂₈H₂₇F₂N₅O₄, 535.20; m/z found, 536.2 [M+H]⁺. ¹H NMR (400 MHz, DMSO-d₆) δ 10.98 (s, 1H), 8.02 (s, 1H), 7.67 (s, 1H), 7.61 (s, 1H), 7.50 (d, J=8.0 Hz, 1H), 7.40 (d, J=9.6 Hz, 1H), 7.32 (d, J=7.6 Hz, 1H), 5.11-5.07 (m, 1H), 4.87 (d, J=9.8 Hz, 1H), 4.55 (s, 1H), 4.46-4.35 (m, 1H), 4.28-4.19 (m, 1H), 4.04 (s, 3H), 3.75 (s, 2H), 3.13-3.07 (m, 1H), 2.90-2.85 (m, 2H), 2.62-2.56 (m, 2H), 2.42-2.35 (m, 1H), 2.29-2.27 (m, 1H), 2.18-2.15 (m, 1H), 1.99-1.92 (m, 2H).

### Compound B39: (3S)-3-(3′,3′-difluoro-1′-(3-(1-methyl-1H-pyrazol-4-yl) benzyl)-6-oxo-6,8-dihydro-2H,7H-spiro[furo[2,3-e]isoindole-3,4′-piperidin]-7-yl) piperidine-2,6-dione

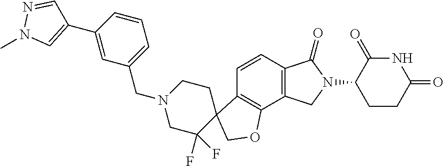 [SMILES: Cn1cc(-c2cccc(CN3CCC4(COc5c4ccc4c5CN([C@H]5CCC(=O)NC5=O)C4=O)C(F)(F)C3)c2)cn1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 3-(1-methyl-1H-pyrazol-4-yl)benzaldehyde (Intermediate B27) as a white solid. LC-MS (ESI): mass calcd. for C₃₀H₂₉F₂N₅O₄, 561.22; m/z found, 562.2 [M+H]⁺. ¹H NMR (400 MHz, DMSO-d₆) δ 10.98 (d, J=4.0 Hz, 1H), 8.13 (s, 1H), 7.85 (s, 1H), 7.49 (dd, J=13.6, 7.2 Hz, 3H), 7.37-7.31 (m, 2H), 7.17 (d, J=7.6 Hz, 1H), 5.12-5.08 (m, 1H), 4.87 (dd, J=9.8, 5.8 Hz, 1H), 4.57 (d, J=8.4 Hz, 1H), 4.41 (t, J=16.8 Hz, 1H), 4.24 (t, J=16.8 Hz, 1H), 3.87 (s, 3H), 3.70-3.61 (m, 2H), 3.13-3.10 (m, 1H), 2.96-2.84 (m, 2H), 2.68-2.56 (m, 2H), 2.43-2.41 (m, 1H), 2.34-2.26 (m, 1H), 2.16 (t, J=11.8 Hz, 1H), 2.02-1.91 (m, 2H).

### Compound B40: (3S)-3-(1′-((1H-indazol-5-yl) methyl)-3′,3′-difluoro-6-oxo-6,8-dihydro-2H,7H-spiro[furo[2,3-e]isoindole-3,4′-piperidin]7-yl) piperidine-2,6-dione

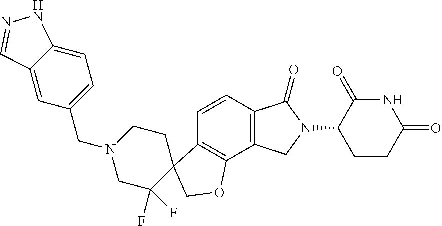 [SMILES: O=C1CC[C@H](N2Cc3c(ccc4c3OCC43CCN(Cc4ccc5[nH]ncc5c4)CC3(F)F)C2=O)C(=O)N1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1H-indazole-5-carbaldehyde as a white solid. LC-MS (ESI): mass calcd. for C₂₇H₂₅F₂N₅O₄, 521.19; m/z found, 522.2 [M+H]⁺. ¹H NMR (400 MHz, DMSO-d₆) δ 13.04 (s, 1H), 10.97 (s, 1H), 8.05 (s, 1H), 7.68 (s, 1H), 7.54-7.48 (m, 2H), 7.35 (d, J=8.6 Hz, 1H), 7.32 (d, J=7.6 Hz, 1H), 5.11-5.07 (m, 1H), 4.86 (dd, J=9.8, 5.6 Hz, 1H), 4.56 (d, J=8.8 Hz, 1H), 4.41 (t, J=16.8 Hz, 1H), 4.24 (t, J=16.8 Hz, 1H), 3.77-3.67 (m, 2H), 3.11 (s, 1H), 2.88 (d, J=11.6 Hz, 2H), 2.65-2.54 (m, 2H), 2.43-2.41 (m, 1H), 2.27 (d, J=11.4 Hz, 1H), 2.15 (t, J=11.4 Hz, 1H), 1.98 (d, J=11.8 Hz, 2H).

### Compound B41: (3S)-3-(1′-((1H-indazol-4-yl)methyl)-3′,3′-difluoro-6-oxo-6,8-dihydro-2H,7H-spiro[furo[2,3-e]isoindole-3,4′-piperidin]-7-yl)piperidine-2,6-dione

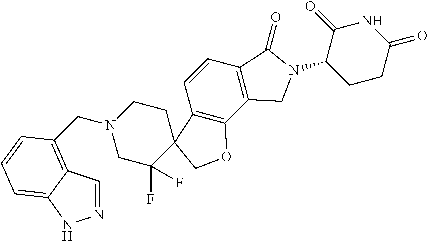 [SMILES: O=C1CC[C@H](N2Cc3c(ccc4c3OCC43CCN(Cc4cccc5[nH]ncc45)CC3(F)F)C2=O)C(=O)N1]

The title compound was prepare according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1H-indazole-4-carbaldehyde as a yellow solid. LC-MS (ESI): mass calcd. for C₂₇H₂₅F₂N₅O₄, 521.19; m/z found, 522.4 [M+H]⁺. ¹H NMR (400 MHz, DMSO-d₆) δ 13.07 (s, 1H), 10.97 (s, 1H), 8.28 (s, 1H), 7.52 (d, J=8.0 Hz, 1H), 7.46 (d, J=8.2 Hz, 1H), 7.34-7.28 (m, 2H), 7.06 (d, J=7.0 Hz, 1H), 5.14-5.05 (m, 1H), 4.90-4.86 (m, 1H), 4.57 (d, J=8.2 Hz, 1H), 4.41 (t, J=16.4 Hz, 1H), 4.24 (t, J=16.4 Hz, 1H), 3.96 (s, 2H), 3.20-3.13 (m, 1H), 2.90-2.86 (m, 2H), 2.75-2.54 (m, 2H), 2.46-2.35 (m, 1H), 2.29-2.16 (m, 2H), 2.01-1.95 (m, 2H).

### Compound B42: (3S)-3-(3′,3′-difluoro-1′-((2-methyl-2H-indazol-4-yl)methyl)-6-oxo-6,8-dihydro-2H,7H-spiro[furo[2,3-e]isoindole-3,4′-piperidin]-7-yl)piperidine-2,6-dione

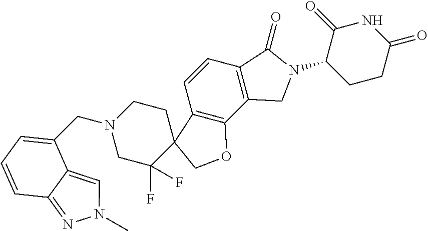 [SMILES: Cn1cc2c(CN3CCC4(COc5c4ccc4c5CN([C@H]5CCC(=O)NC5=O)C4=O)C(F)(F)C3)cccc2n1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 2-methyl-1H-indazole-4-carbaldehyde (Intermediate B33b) as a yellow solid. LC-MS (ESI): mass calcd. for C₂₈H₂₇F₂N₅O₄, 535.20; m/z found, 536.4 [M+H]⁺. ¹H NMR (400 MHz, DMSO-d₆) δ 8.45 (s, 1H), 7.55-7.48 (m, 2H), 7.33 (d, J=7.8 Hz, 1H), 7.19 (dd, J=8.6, 6.8 Hz, 1H), 6.97 (d, J=6.8 Hz, 1H), 5.15-5.05 (m, 1H), 4.88-4.85 (m, 1H), 4.58-456 (m, 1H), 4.47-4.36 (m, 1H), 4.27-4.24 (m, 1H), 4.20 (s, 3H), 3.87 (s, 2H), 3.18-3.15 (m, 1H), 2.95-2.82 (m, 2H), 2.70 (t, J=10.2 Hz, 1H), 2.62-2.56 (m, 1H), 2.49-2.40 (m, 1H), 2.31-2.15 (m, 2H), 1.99-1.95 (m, 2H).

### Compound B43: (3S)-3-(3′,3′-difluoro-1′-((1-methyl-1H-indazol-4-yl)methyl)-6-oxo-6,8-dihydro-2H,7H-spiro[furo[2,3-e]isoindole-3,4′-piperidin]-7-yl)piperidine-2,6-dione

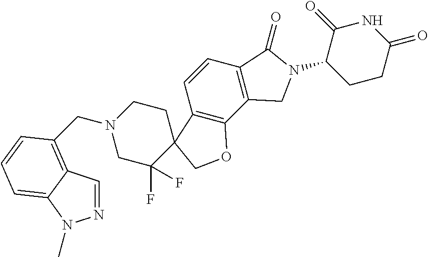 [SMILES: Cn1ncc2c(CN3CCC4(COc5c4ccc4c5CN([C@H]5CCC(=O)NC5=O)C4=O)C(F)(F)C3)cccc21]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1-methyl-1H-indazole-4-carbaldehyde (Intermediate B33a) as a yellow solid. LC-MS (ESI): mass calcd. for C₂₈H₂₇F₂N₅O₄, 535.20; m/z found, 536.5 [M+H]⁺. ¹H NMR (400 MHz, DMSO-d₆) δ 10.98 (d, J=3.8 Hz, 1H), 8.27 (s, 1H), 7.59 (d, J=8.0 Hz, 1H), 7.51 (d, J=8.0 Hz, 1H), 7.38 (t, J=7.6 Hz, 1H), 7.33 (d, J=7.6 Hz, 1H), 7.12 (d, J=6.4 Hz, 1H), 5.17-5.03 (m, 1H), 4.93-4.85 (m, 1H), 4.53-4.50 (m, 1H), 4.46-4.36 (m, 1H), 4.27-4.20 (m, 1H), 4.06 (s, 3H), 3.53-3.49 (m, 2H), 3.36-3.15 (m, 2H), 3.03-2.79 (m, 3H), 2.62-2.56 (m, 1H), 2.46-2.38 (m, 1H), 2.32-2.29 (m, 1H), 2.00-1.97 (m, 2H).

### Compound B44. 3-(5-(2-methyl-7-(pyrrolidin-1-ylmethyl)oxazolo[4,5-b]pyridin-5-yl)-1-oxoisoindolin-2-yl)piperidine-2,6-dione

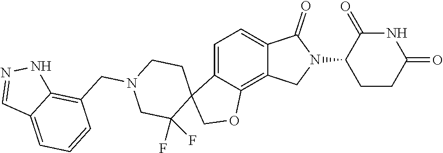 [SMILES: O=C1CC[C@H](N2Cc3c(ccc4c3OCC43CCN(Cc4cccc5cn[nH]c45)CC3(F)F)C2=O)C(=O)N1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1H-indazole-7-carbaldehyde as a white solid. LC-MS (ESI): mass calcd. for C₂₇H₂₅F₂N₅O₄, 521.52; m/z found, 522.2 [M+H]⁺. ¹H NMR (400 MHz, DMSO-d₆) δ 13.02 (s, 1H), 10.98 (s, 1H), 8.11 (s, 1H), 7.71 (d, J=7.8 Hz, 1H), 7.50 (d, J=7.6 Hz, 1H), 7.33-7.27 (m, 2H), 7.15-7.05 (m, 1H), 5.14-5.04 (m, 1H), 4.88-4.85 (m, 1H), 4.57-4.45 (m, 1H), 4.41-4.36 (m, 1H), 4.28-4.20 (m, 1H), 4.00-3.87 (m, 2H), 3.22-3.17 (m, 1H), 3.01-2.84 (m, 2H), 2.72-2.55 (m, 2H), 2.46-2.17 (m, 3H), 2.02-1.87 (m, 2H).

### Compound B45: (3S)-3-(3′,3′-difluoro-1′-((1-methyl-1H-indazol-7-yl)methyl)-6-oxo-6,8-dihydro-2H,7H-spiro[furo[2,3-e]isoindole-3,4′-piperidin]-7-yl)piperidine-2,6-dione

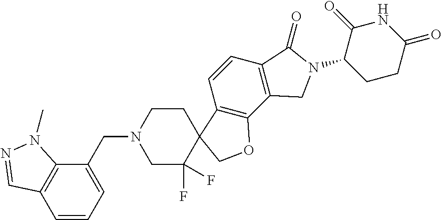 [SMILES: Cn1ncc2cccc(CN3CCC4(COc5c4ccc4c5CN([C@H]5CCC(=O)NC5=O)C4=O)C(F)(F)C3)c21]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1-methyl-1H-indazole-7-carbaldehyde (Intermediate B31a) as a white solid. LC-MS (ESI): mass calcd. for C₂₈H₂₇F₂N₅O₄, 535.55; m/z found, 536.2 [M+H]⁺. ¹H NMR (400 MHz, DMSO-d₆) δ 10.98 (s, 1H), 8.03 (s, 1H), 7.72 (d, J=7.4 Hz, 1H), 7.49 (d, J=7.8 Hz, 1H), 7.30 (d, J=7.8 Hz, 1H), 7.21 (d, J=6.8 Hz, 1H), 7.08-7.04 (m, 1H), 5.12-5.06 (m, 1H), 4.95-4.91 (m, 1H), 4.64-4.55 (m, 1H), 4.46-4.36 (m, 4H), 4.28-4.20 (m, 1H), 4.10-4.05 (m, 1H), 3.94-3.89 (m, 1H), 3.19-3.14 (m, 1H), 2.97-2.72 (m, 3H), 2.62-2.55 (m, 1H), 2.45-2.40 (m, 1H), 2.25-2.14 (m, 2H), 2.06-1.86 (m, 2H).

### Compound B46: (3S)-3-(3′,3′-difluoro-6-oxo-1′-((1-((tetrahydro-2H-pyran-4-yl)methyl)-1H-pyrazol-4-yl)methyl)-6,8-dihydro-2H,7H-spiro[furo[2,3-e]isoindole-3,4′-piperidin]-7-yl)piperidine-2,6-dione

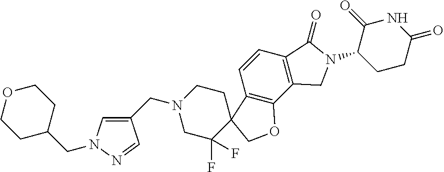 [SMILES: O=C1CC[C@H](N2Cc3c(ccc4c3OCC43CCN(Cc4cnn(CC5CCOCC5)c4)CC3(F)F)C2=O)C(=O)N1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1-((tetrahydro-2H-pyran-4-yl)methyl)-1H-pyrazole-4-carbaldehyde (Intermediate B17) as a white solid. LC-MS (ESI): mass calced for: C₂₉H₃₃F₂N₅O₅ 569.24; m/z found, 570.4 [M+H]⁺.

¹H NMR (400 MHz, DMSO-d₆) δ 10.98 (s, 1H), 7.64 (s, 1H), 7.46 (d, J=7.6 Hz, 1H), 7.37 (s, 1H), 7.31 (d, J=7.6 Hz, 1H), 5.11-5.07 (m, 1H), 4.86-4.78 (m, 1H), 4.51 (s, 1H), 4.41 (t, J=17.0 Hz, 1H), 4.23 (t, J=17.0 Hz, 1H), 3.97 (d, J=7.21 Hz, 2H), 3.82 (d, J=7.6 Hz, 2H), 3.52 (s, 2H), 3.29-3.21 (m, 3H), 3.11 (s, 1H), 2.96-2.80 (m, 2H), 2.61-2.56 (m, 1H), 2.45-2.36 (m, 1H), 2.31-2.18 (m, 1H), 2.05-1.93 (m, 4H), 1.35 (d, J=11.2 Hz, 2H), 1.28-1.15 (m, 2H). ¹⁹F NMR (376 MHz, DMSO) δ −107.72 (s), −108.36 (s).

### Compound B47: (3S)-3-(1′-((1-((1,1-dioxidotetrahydro-2H-thiopyran-4-yl)methyl)-1H-pyrazol-4-yl)methyl)-3′,3′-difluoro-6-oxo-6,8-dihydro-2H,7H-spiro[furo[2,3-e]isoindole-3,4′-piperidin]-7-yl)piperidine-2,6-dione

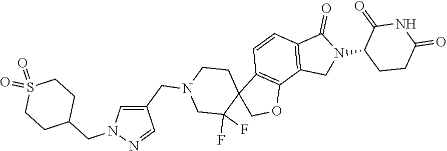 [SMILES: O=C1CC[C@H](N2Cc3c(ccc4c3OCC43CCN(Cc4cnn(CC5CCS(=O)(=O)CC5)c4)CC3(F)F)C2=O)C(=O)N1]

The title compound was prepared according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 1-((1,1-dioxidotetrahydro-2H-thiopyran-4-yl)methyl)-1H-pyrazole-4-carbaldehyde (Intermediate B34) as a white solid. LC-MS (ESI): mass calcd. for C₂₉H₃₃F₂N₅O₆S, 617.0; m/z found, 618.2 [M+H]⁺. ¹H NMR (400 MHz, DMSO-d₆) δ 10.99 (d, J=4.4 Hz, 1H), 7.82 (s, 1H), 7.56 (s, 1H), 7.46 (d, J=7.6 Hz, 1H), 7.37 (d, J=7.6 Hz, 1H), 5.14-5.07 (m, 1H), 4.92 (dd, J=9.8, 5.2 Hz, 1H), 4.63 (s, 1H), 4.45 (t, J=17.2 Hz, 1H), 4.28 (t, J=17.2 Hz, 1H), 4.11 (d, J=7.0 Hz, 4H), 3.65 (s, 1H), 3.26 (s, 2H), 3.14 (dd, J=19.0, 6.8 Hz, 2H), 3.03 (d, J=12.6 Hz, 2H), 2.90 (d, J=11.8 Hz, 1H), 2.71 (s, 1H), 2.60 (d, J=16.6 Hz, 1H), 2.41 (dd, J=15.6, 8.4 Hz, 2H), 2.18 (s, 2H), 2.04-1.95 (m, 1H), 1.84 (d, J=12.4 Hz, 2H), 1.70-1.64 (m, 2H).

### Compound B48: (3S)-3-(3′,3′-difluoro-1′-((3-(1-(methyl-d3)-1H-pyrazol-4-yl)phenyl)methyl-d2)-6-oxo-6,8-dihydro-2H,7H-spiro[furo[2,3-e]isoindole-3,4′-piperidin]-7-yl)piperidine-2,6-dione

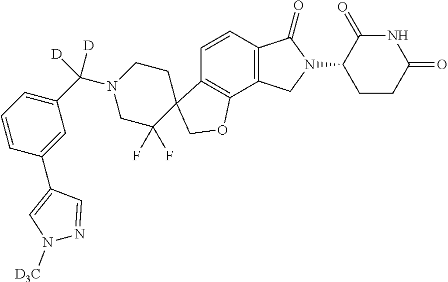 [SMILES: [2H]C([2H])(c1cccc(-c2cnn(C([2H])([2H])[2H])c2)c1)N1CCC2(COc3c2ccc2c3CN([C@H]3CCC(=O)NC3=O)C2=O)C(F)(F)C1]

The title compound was prepared according to the procedure described in Compound B-7 by displacement between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 4-(3-(bromomethyl-d2)phenyl)-1-(methyl-d3)-1H-pyrazole (Intermediate B20) as a white solid. LC-MS (ESI): mass calcd. for C₃₀H₂₄D₅F₂N₅O₄, 566.25; m/z found, 567.2 [M+H]⁺. ¹H NMR (400 MHz, DMSO-d₆) δ 10.98 (s, 1H), 8.13 (s, 1H), 7.85 (s, 1H), 7.49 (dd, J=13.6, 7.2 Hz, 3H), 7.38-7.30 (m, 2H), 7.17 (d, J=7.4 Hz, 1H), 5.11-5.06 (m, 1H), 4.87 (d, J=10.2 Hz, 1H), 4.55 (d, J=9.2 Hz, 1H), 4.39 (d, J=17.4 Hz, 1H), 4.26 (d, J=17.4 Hz, 1H), 3.17-3.09 (m, 1H), 2.89-2.85 (m, 2H), 2.62-2.56 (m, 2H), 2.46-2.38 (m, 1H), 2.28 (d, J=12.8 Hz, 1H), 2.23-2.12 (m, 1H), 2.03-1.88 (m, 2H). ¹⁹F NMR (376 MHz, DMSO-d₆) δ −107.96 (s), −108.60 (s).

### Compound B49: (3S)-3-(3′,3′-difluoro-1′-(3-(1-(methyl-d3)-1H-pyrazol-4-yl) benzyl)-6-oxo-6,8-dihydro-2H,7H-spiro[furo[2,3-e]isoindole-3,4′-piperidin]-7-yl) piperidine-2,6-dione

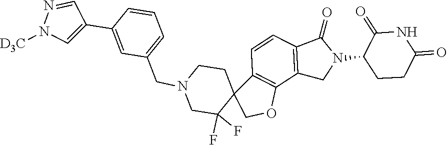 [SMILES: [2H]C([2H])([2H])n1cc(-c2cccc(CN3CCC4(COc5c4ccc4c5CN([C@H]5CCC(=O)NC5=O)C4=O)C(F)(F)C3)c2)cn1]

The title compound was prepare according to the procedure described in Compound B-1 by reductive amination between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 3-(1-(methyl-d3)-1H-pyrazol-4-yl)benzaldehyde (Intermediate B23) as a white solid. LC-MS (ESI): mass calcd. for C₃₀H₂₆D₃F₂N₅O₄, 564.24; m/z found, 565.3 [M+H]⁺. ¹H NMR (400 MHz, DMSO-d₆) δ 10.97 (s, 1H), 8.13 (s, 1H), 7.85 (s, 1H), 7.49 (dd, J=13.8, 7.0 Hz, 3H), 7.33 (dd, J=7.6, 3.2 Hz, 2H), 7.17 (d, J=7.4 Hz, 1H), 5.11-5.07 (m, 1H), 4.91-4.83 (m, 1H), 4.57 (t, J=8.4 Hz, 1H), 4.41 (t, J=16.8 Hz, 1H), 4.24 (t, J=16.8 Hz, 1H), 3.66 (s, 2H), 3.14-3.11 (m, 1H), 2.89-2.86 (m, 2H), 2.62-2.56 (m, 2H), 2.44-2.38 (m, 1H), 2.29 (d, J=9.6 Hz 1H), 2.16 (t, J=9.0 Hz, 1H), 2.01-1.93 (m, 2H).

### Compound B50: (3S)-3-(3′,3′-difluoro-1′-((3-(1-methyl-1H-pyrazol-4-yl)phenyl)methyl-d2)-3-oxo-1,3,7,8-tetrahydro-2H-spiro[cyclopenta[e]isoindole-6,4′-piperidin]-2-yl)piperidine-2,6-dione

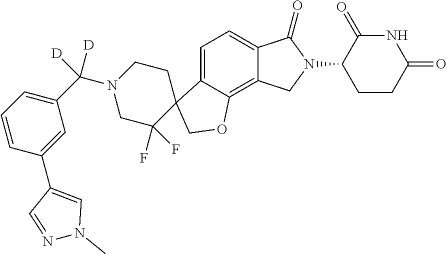 [SMILES: [2H]C([2H])(c1cccc(-c2cnn(C)c2)c1)N1CCC2(COc3c2ccc2c3CN([C@H]3CCC(=O)NC3=O)C2=O)C(F)(F)C1]

The title compound was prepared according to the procedure described in Compound B-7 by displacement between (3S)-3-(3′,3′-difluoro-6-oxo-6,8-dihydro-2H,7H-spiro[furo[2,3-e]isoindole-3,4′-piperidin]-7-yl)piperidine-2,6-dione (Intermediate B11) and 4-(3-(bromomethyl-d2)phenyl)-1-methyl-1H-pyrazole (Intermediate B22) as a white solid. LC-MS (ESI): mass calcd. for C₃0H₂₇D₂N₅O₄, 563.60; m/z found, 564.6 [M+H]⁺. ¹H NMR (400 MHz, DMSO-d₆) δ 10.98 (s, 1H), 8.13 (s, 1H), 7.85 (s, 1H), 7.49 (dd, J=13.6, 6.8 Hz, 3H), 7.37-7.30 (m, 2H), 7.17 (d, J=7.6 Hz, 1H), 5.11-5.06 (m, 1H), 4.88 (d, J=9.8 Hz, 1H), 4.55 (d, J=8.8 Hz, 1H), 4.47-4.34 (m, 1H), 4.31-4.18 (m, 1H), 3.87 (s, 3H), 3.14-3.12 (m, 1H), 2.89-2.85 (m, 2H), 2.68-2.57 (m, 2H), 2.43-2.25 (m, 2H), 2.16 (t, J=11.8 Hz, 1H), 2.04-1.88 (m, 2H).

### Compound B51: (S)-3-((S)-3′,3′-difluoro-1′-(3-(1-methyl-1H-pyrazol-4-yl) benzyl)-6-oxo-6,8-dihydro-2H,7H-spiro[furo[2,3-e]isoindole-3,4′-piperidin]-7-yl) piperidine-2,6-dione (PVT-0006571-001)

### Compound B52: (S)-3-((R)-3′,3′-difluoro-1′-(3-(1-methyl-1H-pyrazol-4-yl) benzyl)-6-oxo-6,8-dihydro-2H,7H-spiro[furo[2,3-e]isoindole-3,4′-piperidin]-7-yl)piperidine-2,6-dione

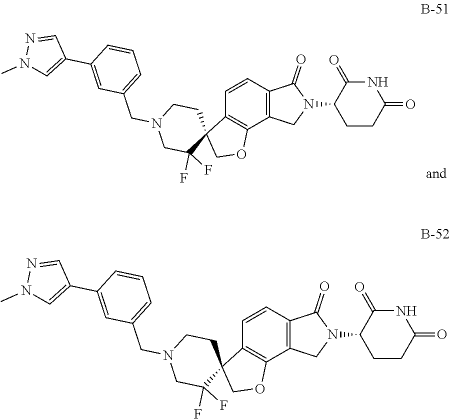 [SMILES: Cn1cc(-c2cccc(CN3CC[C@]4(COc5c4ccc4c5CN([C@H]5CCC(=O)NC5=O)C4=O)C(F)(F)C3)c2)cn1 ; Cn1cc(-c2cccc(CN3CC[C@@]4(COc5c4ccc4c5CN([C@H]5CCC(=O)NC5=O)C4=O)C(F)(F)C3)c2)cn1]

(3S)-3-(3′,3′-difluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione (100 mg, 178 μmol, 1.0 eq) was purified by chiral Prep-HPLC with ChiralPak 1H (5 μm, 250×21.2 mm), and mobile phase of A for CO₂ and B for ETOH (0.1% NH₃H₂O) 5-99% over 3 h at a flow rate of 40 mL/min to give (S)-3-((S)-3′,3′-difluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione (9.60 mg, yield 9%) as a white solid. (e.e.: 42.20%) and (S)-3-((R)-3′,3′-difluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione (10.0 mg, yield 10.0%) as a white solid. (e.e.:54.64%). [0552](S)-3-((S)-3′,3′-difluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione: LC-MS (ESI): mass calcd. for C₃₀H₂₉F₂N₅O₄, 561.22; m/z found, 562.2 [M+H]⁺. ¹H NMR (400 MHz, DMSO-d₆) δ 10.99 (s, 1H), 8.14 (s, 1H), 7.85 (s, 1H), 7.49 (dd, J=13.8, 7.2 Hz, 3H), 7.37-7.31 (m, 2H), 7.17 (d, J=7.6 Hz, 1H), 5.11-5.06 (m, 1H), 4.87 (dd, J=9.8, 6.2 Hz, 1H), 4.55 (d, J=8.8 Hz, 1H), 4.46-4.36 (m, 1H), 4.29-4.19 (m, 1H), 3.87 (s, 3H), 3.70-3.62 (m, 2H), 3.13 (s, 1H), 2.96-2.84 (m, 2H), 2.68-2.57 (m, 2H), 2.45-2.40 (m, 1H), 2.33-2.26 (m, 1H), 2.16 (t, J=11.8 Hz, 1H), 2.01-1.91 (m, 2H).

(S)-3-((R)-3′,3′-difluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione: LC-MS (ESI): mass calcd. for C₃₀H₂₉F₂N₅O₄, 561.22; m/z found, 562.2 [M+H]⁺. ¹H NMR (400 MHz, DMSO-d₆) δ 10.98 (s, 1H), 8.14 (s, 1H), 7.85 (s, 1H), 7.49 (dd, J=13.8, 7.2 Hz, 3H), 7.36-7.30 (m, 2H), 7.17 (d, J=7.8 Hz, 1H), 5.13-5.08 (m, 1H), 4.87 (d, J=10.0 Hz, 1H), 4.57 (d, J=9.6 Hz, 1H), 4.41 (t, J=16.8 Hz, 1H), 4.24 (t, J=16.8 Hz, 1H), 3.87 (s, 3H), 3.66 (s, 2H), 3.11 (s, 1H), 2.90-2.86 (m, 2H), 2.65-2.57 (m, 2H), 2.43-2.38 (m, 1H), 2.29 (d, J=11.0 Hz, 1H), 2.16 (t, J=11.8 Hz, 1H), 2.02-1.94 (m, 2H).

### Compound B53: (S)-3-((S)-3′,3′-difluoro-1′-(3-(1-(oxetan-3-yl)-1H-pyrazol-4-yl)benzyl)-6-oxo-6,8-dihydro-2H,7H-spiro[furo[2,3-e]isoindole-3,4′-piperidin]-7-yl)piperidine-2,6-dione

### Compound B54: (S)-3-((R)-3′,3′-difluoro-1′-(3-(1-(oxetan-3-yl)-1H-pyrazol-4-yl)benzyl)-6-oxo-6,8-dihydro-2H,7H-spiro[furo[2,3-e]isoindole-3,4′-piperidin]-7-yl)piperidine-2,6-dione

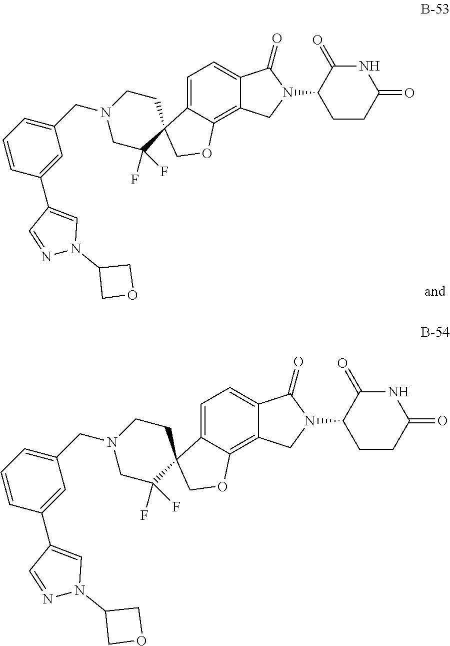 [SMILES: O=C1CC[C@H](N2Cc3c(ccc4c3OC[C@]43CCN(Cc4cccc(-c5cnn(C6COC6)c5)c4)CC3(F)F)C2=O)C(=O)N1 ; O=C1CC[C@H](N2Cc3c(ccc4c3OC[C@@]43CCN(Cc4cccc(-c5cnn(C6COC6)c5)c4)CC3(F)F)C2=O)C(=O)N1]

The racemate product (3S)-3-(3′,3′-difluoro-1′-(3-(1-(oxetan-3-yl)-1H-pyrazol-4-yl)benzyl)-6-oxo-6,8-dihydro-2H,7H-spiro[furo[2,3-e]isoindole-3,4′-piperidin]-7-yl)piperidine-2,6-dione (PVT-0006340-001) (400 mg) was separated by SFC Prep-HPLC with Waters Thar 80 preparative SFC (ChiralCel OJ, 5 μm, 250×21.2 mm I.D., 5 μm), and mobile phase of A for CO₂ and B for 0.1% 7 mol/L NH3 in MeOH over 3 h, at a flow rate of 40 mL/min to give (S)-3-((S)-3′,3‘-difluoro-1’-(3-(1-(oxetan-3-yl)-1H-pyrazol-4-yl) benzyl)-6-oxo-6,8-dihydro-2H,7H-spiro[furo[2,3-e]isoindole-3,4′-piperidin]-7-yl) piperidine-2,6-dione (33.5 mg, yield 8.37%) as a white solid and (S)-3-((R)-3′,3′-difluoro-1′-(3-(1-(oxetan-3-yl)-1H-pyrazol-4-yl) benzyl)-6-oxo-6,8-dihydro-2H,7H-spiro[furo[2,3-e]isoindole-3,4′-piperidin]-7-yl) piperidine-2,6-dione (59.5 mg, 14.9%) as a white solid.

(S)-3-((S)-3′,3′-difluoro-1′-(3-(1-(oxetan-3-yl)-1H-pyrazol-4-yl) benzyl)-6-oxo-6,8-dihydro-2H,7H-spiro[furo[2,3-e]isoindole-3,4′-piperidin]-7-yl) piperidine-2,6-dione (B-53): LC-MS (ESI): mass calcd. for C₃₂H₃₁F₂N₅O₅, 603.23; m/z found, 604.3[M+H]⁺. ¹H NMR (400 MHz, DMSO-d₆) δ 10.99 (s, 1H), 8.38 (s, 1H), 8.14 (s, 1H), 8.03 (s, 1H), 7.58-7.48 (m, 3H), 7.35 (dd, J=20.2, 7.6 Hz, 2H), 7.21 (d, J=7.6 Hz, 1H), 5.64-5.55 (m, 1H), 5.10-5.07 (m, 1H), 4.98-4.91 (m, 4H), 4.88 (d, J=10.0 Hz, 1H), 4.56 (d, J=10.0 Hz, 1H), 4.39 (d, J=16.8 Hz, 1H), 4.29-4.19 (m, 1H), 3.68 (s, 2H), 3.14 (s, 1H), 2.89 (d, J=12.4 Hz, 2H), 2.62 (t, J=20.6 Hz, 2H), 2.43 (d, J=12.0 Hz, 1H), 2.29 (d, J=11.9 Hz, 1H), 2.13 (d, J=42.3 Hz, 1H), 1.97 (s, 2H).

(S)-3-((R)-3′,3′-difluoro-1′-(3-(1-(oxetan-3-yl)-1H-pyrazol-4-yl) benzyl)-6-oxo-6,8-dihydro-2H,7H-spiro[furo[2,3-e]isoindole-3,4′-piperidin]-7-yl) piperidine-2,6-dione (B-54): LC-MS (ESI): mass calcd. C₃₂H₃₁F₂N₅O₅, 603.23; m/z found, 604.3 [M+H]⁺. ¹H NMR (400 MHz, DMSO-d₆) δ 10.97 (s, 1H), 8.38 (s, 1H), 8.13 (s, 1H), 8.02 (s, 1H), 7.53 (dd, J=16.8, 7.6 Hz, 3H), 7.35 (dd, J=17.8, 8.0 Hz, 2H), 7.20 (d, J=7.0 Hz, 1H), 5.65-5.54 (m, 1H), 5.11-5.07 (m, 1H), 4.98-4.90 (m, 4H), 4.87 (d, J=9.8 Hz, 1H), 4.57 (d, J=9.2 Hz, 1H), 4.43 (d, J=17.6 Hz, 1H), 4.22 (d, J=16.7 Hz, 1H), 3.67 (s, 2H), 3.12 (s, 1H), 2.91 (t, J=13.4 Hz, 2H), 2.62 (t, J=20.6 Hz, 2H), 2.41 (d, J=13.4 Hz, 1H), 2.35-2.25 (m, 1H), 2.18 (d, J=11.8 Hz, 1H), 1.98 (dd, J=9.8, 7.1 Hz, 2H).

### Example B55: (S)-3-(4-fluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione

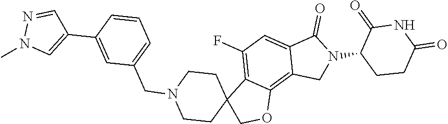 [SMILES: Cn1cc(-c2cccc(CN3CCC4(CC3)COc3c5c(cc(F)c34)C(=O)N([C@H]3CCC(=O)NC3=O)C5)c2)cn1]

### Step A: tert-butyl (S)-5-amino-4-(4-fluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)-5-oxopentanoate

A solution of tert-butyl (S)-5-amino-4-(4-fluoro-6-oxo-6,8-dihydro-2H,7H-spiro[furo[2,3-e]isoindole-3,4′-piperidin]-7-yl)-5-oxopentanoate (Intermediate 36, 60 mg, 1.0 eq.) and 3-(1-methyl-1H-pyrazol-4-yl)benzaldehyde (23 mg, 10 eq.) in DMAc (3 mL) was added acetic acid (0.01 mL). The resulting reaction mixture was stirred at ambient temperature for 15 min. Then sodium triacetoxyborohydride (57 mg, 2 eq.) was added. The resulting reaction mixture was stirred at room temperature for 12 h. The reaction mixture was then diluted with water (100 mL) and extracted with DCM/MeOH=10:1 (x3). The organic phases were combined and dried over anhydrous Na₂SO₄. The solvent was removed under reduced pressure. The resulting crude material was purified by flash chromatography to afford the title product as a white solid (60 mg). LC-MS (m/z): [M+H]⁺=618.40.

### Step B: (S)-3-(4-fluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)piperidine-2,6-dione

To the suspension of tert-butyl (S)-5-amino-4-(4-fluoro-1′-(3-(1-methyl-1H-pyrazol-4-yl)benzyl)-6-oxo-6,8-dihydro-2H,7H-spiro[furo[2,3-e]isoindole-3,4′-piperidin]-7-yl)-5-oxopentanoate (60 mg, 1 eq) in ACN (1 mL) was added Benzenesulfonic acid (61 mg, 4 eq). The resulting suspension was stirred at 90° C. for 5 h. The reaction mixture was allowed to cool to room temperature and added TFA, then purified by prep-HPLC quickly to afford a white solid as the final product (27 mg). LC-MS (m/z): [M+H]⁺=544.40.

### Example B56: (S)-3-(1-benzyl-6′-oxo-1′,2′,6′,8′-tetrahydro-7′H-spiro[piperidine-4,3′-pyrrolo[3,4-g]indol]-7′-yl)piperidine-2,6-dione

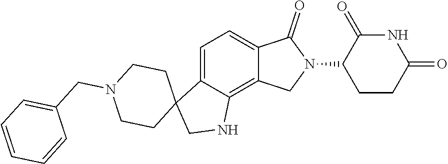

### Step A: tert-butyl (S)-5-amino-4-(1-benzyl-6′-oxo-1′,2′,6′,8′-tetrahydro-7′H-spiro[piperidine-4,3′-pyrrolo[3,4-g]indol]-7′-yl)-5-oxopentanoate

A solution of tert-butyl (S)-5-amino-5-oxo-4-(6′-oxo-1′,2′,6′,8′-tetrahydro-7′H-spiro[piperidine-4,3′-pyrrolo[3,4-g]indol]-7′-yl)pentanoate (Intermediate 37, 15 mg, 1.0 eq.) and benzaldehyde (4.50 mg, 1.2 eq.) in DMAc (3 mL) was added acetic acid (0.01 mL). The resulting reaction mixture was stirred at ambient temperature for 15 min. Then sodium triacetoxyborohydride(15 mg, 2 eq.) was added. The resulting reaction mixture was stirred at room temperature for 3 h. The reaction mixture was then diluted with water and extracted with DCM/MeOH=10:1(3×). The organic phases were combined and dried over anhydrous Na-₂SO₄. The solvent was removed under reduced pressure. The resulting crude material was purified by flash chromatography to afford the title compound (10 mg).

### Step B: (S)-3-(1-benzyl-6′-oxo-1′,2′,6′,8′-tetrahydro-7′H-spiro[piperidine-4,3′-pyrrolo[3,4-g]indol]-7′-yl)piperidine-2,6-dione

To the suspension of tert-butyl (S)-5-amino-4-(1-benzyl-6′-oxo-1′,2′,6′,8′-tetrahydro-7′H-spiro[piperidine-4,3′-pyrrolo[3,4-g]indol]-7′-yl)-5-oxopentanoate (10 mg, 1 eq) in CH₃CN (1 mL) was added Benzenesulfonic acid (12 mg, 4 eq). The resulting suspension was stirred at 90° C. for 5 h, The reaction mixture was allowed to cool to room temperature and TFA (50 mg) was added. Then the mixture was purified by prep-HPLC quickly to afford the title compound as a white solid. LC-MS (m/z): [M+H]⁺=445.2.

### Example B57: (S)-3-(1-(3-(1-methyl-1H-pyrazol-4-yl)benzyl)-6′-oxo-1′,2′,6′,8′-tetrahydro-7′H-spiro[piperidine-4,3′-pyrrolo[3,4-g]indol]-7′-yl)piperidine-2,6-dione

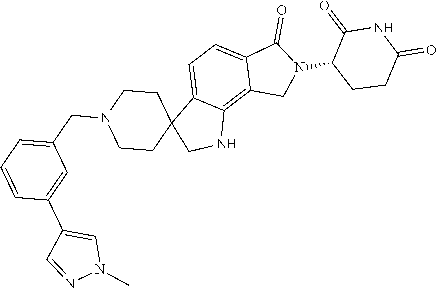 [SMILES: Cn1cc(-c2cccc(CN3CCC4(CC3)CNc3c4ccc4c3CN([C@H]3CCC(=O)NC3=O)C4=O)c2)cn1]

The title compound was prepared according to the procedure described in Compound B-56 by reductive amination between tert-butyl (S)-5-amino-5-oxo-4-(6′-oxo-1′,2′,6′,8′-tetrahydro-7′H-spiro[piperidine-4,3′-pyrrolo[3,4-g]indol]-7′-yl)pentanoate (Intermediate B37) and 3-(1-methyl-1H-pyrazol-4-yl) benzaldehyde (Intermediate B27) followed by acid catalyzed ring cyclization as a white solid. LC-MS (m/z): [M+H]+=525.3.

### Example B58: (S)-4-(4-((7′-(2,6-dioxopiperidin-3-yl)-6′-oxo-1′,6′,7′,8′-tetrahydro-2′H-spiro[piperidine-4,3′-pyrrolo[3,4-g]indol]-1-yl)methyl)-1H-pyrazol-1-yl)benzonitrile

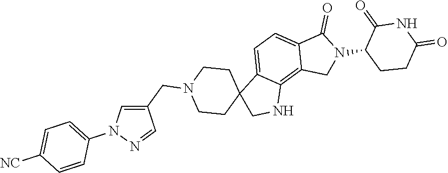 [SMILES: N#Cc1ccc(-n2cc(CN3CCC4(CC3)CNc3c4ccc4c3CN([C@H]3CCC(=O)NC3=O)C4=O)cn2)cc1]

The title compound was prepared according to the procedure described in Compound B-56 by reductive amination between tert-butyl (S)-5-amino-5-oxo-4-(6′-oxo-1′,2′,6′,8′-tetrahydro-7′H-spiro[piperidine-4,3′-pyrrolo[3,4-g]indol]-7′-yl)pentanoate (Intermediate B37) and 4-(4-formyl-1H-pyrazol-1-yl)benzonitrile (Intermediate B24) followed by acid catalyzed ring cyclization as a white solid. LC-MS (m/z): [M+H]⁺=536.2.

### II. Biological Assay

### For Compound A-1 to A-362

### Cereblon Binding Assay

The binding to cereblon (CRBN) was determined using the Cereblon Binding Kit (Cisbio, #64BDCRBNPEG) following the manufacturer's instruction. Briefly, serially diluted compounds were incubated with GST-tagged wild-type human CRBN protein, XL665-labelled Thalidomide and Europium Cryptate labelled GST antibody at room temperature for about 3 hours. Time Resolved Fluorescence Resonance Energy Transfer (TR-FRET) measurements were acquired on a CALRIOstar plate reader with MARS data analysis software (BMG Labtech), with the following settings: 665/10 nm and 620/10 nm emission, 60 μs delay and 400 μs integration. The TR-FRET ratio was taken as the 665/620 nm intensity ratio. The readings were normalized to the control (0.5%) and the IC₅₀ was calculated by nonlinear regression (four parameters sigmoid fitted with variable slope) analysis using the GraphPad Prism 8 software.

### Immunoblotting

Cells were maintained in the appropriate culture medium with 10% FBS at 37° C. and an atmosphere of 5% CO². All the cell lines were used within three months of thawing fresh vials.

Cells were lysed 1× Cell Lysis Buffer (Cell Signaling Technology, #9803), resolved by SDS-PAGE NuPAGE gel (Thermo Fisher Scientific), and transferred to a PVDF membrane (Millipore). Membranes were blocked using Odyssey TBS Blocker Buffer (LI-COR). IRDye 680RD and 800CW Dye-labeled secondary antibodies (LI-COR) were used. The washed membranes were scanned using Odyssey CLx imager (LI-COR). The intensity of Western blot signaling was quantitated using the Odyssey software. Primary antibodies used are: Helios (D8W4X) XP® Rabbit mAb (Cell Signaling Technology, #42427) and GAPDH mouse monoclonal antibody (Santa Cruz Biotechnology, sc-47724).

### IKZF2 HiBiT Assay

Degradation of IKZF2 protein was determined by IKZF2 HiBiT assay using the Jurkat-IKZF2-HiBiT (Promega) cell line. Briefly, cells were seeded in 384-well flat bottom (Corning #07-201-4423595) at a density of 10,000 cells/well in 20 μl of culture medium. Compounds were serially diluted in culture medium, and 20 μl of the diluted compounds were added to the appropriate wells of the plate. After the addition of compounds, the cells were incubated at 37° C. in an atmosphere of 5% CO₂ for 24 hours. At the end of treatment, 40 ul of Nano-Glo HiBiT Lytic Detection Reagent (Promega) was added to each well, and then the plates were incubated at room temperature for 10-20 minutes. The luminescent signal was measured using a CALRIOstar plate reader (BMG Labtech). The readings were normalized to the DMSO-treated cells and the IC₅₀ was calculated by nonlinear regression (four parameters sigmoid fitted with variable slope, least squares fit, and no constraint) analysis using the GraphPad Prism 8 software. Results are shown in Table E2.

TABLE E2
Binding activity and degradation potency:

| | Binding activity | HiBit assay degradation analysis | |
|---|---|---|---|
| No. | IC₅₀ | DC₅₀ | Dₘₐₓ |
| Lenalidomide | C | D | D |
| A1 | D | D | D |
| A2 | C | D | D |
| A3 | B | D | C |
| A4 | | D | C |
| AS | C | D | D |
| A6 | | D | D |
| A7 | | D | D |
| A8 | C | C | C |
| A9 | | D | D |
| A10 | | D | D |
| A11 | B | D | C |
| A12 | B | D | D |
| A13 | | D | D |
| A14 | | D | D |
| A15 | C | C | C |
| A16 | | D | D |
| A17 | | D | C |
| A18 | | D | D |
| A19 | | D | D |
| A20 | | D | D |
| A21 | | D | B |
| A22 | | D | D |
| A23 | | D | D |
| A24 | | D | C |
| A25 | | D | C |
| A26 | | D | D |
| A27 | C | A | A |
| A28 | | D | C |
| A29 | D | D | C |
| A30 | | D | D |
| A31 | C | C | B |
| A32 | | D | D |
| A33 | | D | C |
| A34 | | A | B |
| A35 | | A | A |
| A36 | | B | B |
| A37 | | D | C |
| A38 | | B | B |
| A39 | | A | B |
| A40 | | B | B |
| A41 | | B | B |
| A42 | | D | D |
| A43 | | D | C |
| A44 | | D | D |
| A45 | | C | C |
| A46 | | C | C |
| A47 | | D | C |
| A48 | | D | D |
| A49 | C | A | B |
| A50 | | A | B |
| A51 | | A | B |
| A52 | B | A | B |
| A53 | | D | C |
| A54 | | B | C |
| A56 | C | A | B |
| A57 | | B | B |
| A58 | | D | C |
| A59 | | B | A |
| A60 | | D | C |
| A62 | | C | C |
| A64 | | B | B |
| A65 | | D | C |
| A66 | | D | C |
| A67 | | B | B |
| A68 | | D | C |
| A69 | C | A | B |
| A70 | | C | B |
| A71 | | D | C |
| A72 | | D | C |
| A73 | | D | C |
| A74 | | B | B |
| A75 | | B | B |
| A76 | | D | D |
| A77 | | D | C |
| A78 | | C | C |
| A79 | | D | D |
| A80 | | D | D |
| A81 | | D | D |
| A82 | | D | D |
| A83 | | D | D |
| A84 | | D | C |
| A85 | | D | D |
| A86 | | D | C |
| A87 | C | A | A |
| A88 | | D | D |
| A89 | | B | B |
| A90 | | D | D |
| A91 | | A | B |
| A92 | | B | C |
| A93 | | B | B |
| A94 | | C | C |
| A95 | | D | C |
| A96 | | B | B |
| A97 | | D | C |
| A98 | C | B | B |
| A99 | | D | D |
| A100 | | D | D |
| A101 | | A | B |
| A102 | | C | C |
| A103 | | C | C |
| A104 | | A | A |
| A105 | C | A | B |
| A106 | | A | B |
| A107 | | A | B |
| A108 | | D | C |
| A109 | | A | B |
| A110 | | A | A |
| A111 | | B | B |
| A112 | | C | B |
| A113 | | A | B |
| A114 | | B | B |
| A115 | | B | B |
| A116 | C | A | B |
| A117 | | C | C |
| A118 | | D | C |
| A119 | | A | B |
| A120 | | A | B |
| A121 | | A | B |
| A122 | C | B | B |
| A123 | | D | C |
| A124 | C | C | C |
| A125 | C | A | B |
| A126 | C | C | C |
| A127 | C | A | C |
| A128 | C | B | C |
| A129 | B | A | B |
| A130 | C | A | B |
| A131 | C | B | B |
| A132 | C | A | B |
| A133 | C | A | B |
| A134 | C | A | B |
| A135 | | A | B |
| A136 | | A | C |
| A137 | | A | B |
| A138 | | D | C |
| A139 | | C | B |
| A140 | | D | C |
| A141 | | A | B |
| A142 | | B | B |
| A143 | | A | B |
| A144 | | C | C |
| A145 | B | A | B |
| A146 | | A | B |
| A147 | | A | B |
| A148 | | A | B |
| A149 | | D | D |
| A150 | | B | B |
| A151 | | C | C |
| A152 | | B | B |
| A153 | | B | B |
| A154 | | C | B |
| A155 | | D | C |
| A156 | | D | D |
| A157 | | A | B |
| A158 | | D | D |
| A159 | | D | C |
| A160 | B | A | B |
| A161 | B | A | B |
| A162 | | B | B |
| A163 | | A | B |
| A164 | | B | B |
| A165 | | B | B |
| A166 | | B | C |
| A167 | | B | C |
| A168 | | A | B |
| A169 | | A | B |
| A170 | C | A | B |
| A171 | | B | B |
| A172 | | A | B |
| A173 | | B | B |
| A174 | | A | B |
| A175 | | D | D |
| A176 | | D | C |
| A177 | | A | A |
| A178 | | A | A |
| A179 | | A | A |
| A180 | | D | D |
| A181 | | C | C |
| A182 | | D | C |
| A183 | | C | C |
| A184 | | D | D |
| A185 | | A | B |
| A186 | | A | A |
| A187 | | C | B |
| A188 | | C | B |
| A189 | | B | B |
| A190 | | D | C |
| A191 | | D | D |
| A192 | | D | C |
| A193 | | D | D |
| A194 | | D | D |
| A195 | | D | D |
| A196 | | B | B |
| A197 | | D | D |
| A198 | | C | B |
| A199 | | D | B |
| A200 | | D | D |
| A201 | | D | D |
| A202 | | D | D |
| A203 | | D | D |
| A204 | | D | C |
| A205 | | C | C |
| A206 | | D | D |
| A207 | | D | D |
| A208 | | A | B |
| A209 | | A | B |
| A210 | | D | D |
| A211 | | D | D |
| A212 | | D | C |
| A213 | | A | B |
| A214 | | C | C |
| A215 | | C | C |
| A216 | | B | B |
| A217 | | D | D |
| A218 | | A | B |
| A219 | | C | C |
| A220 | | D | D |
| A221 | | D | C |
| A222 | | D | C |
| A223 | | C | C |
| A224 | | C | C |
| A225 | | D | C |
| A226 | | D | C |
| A227 | | D | C |
| A228 | | D | D |
| A229 | | D | C |
| A230 | | D | C |
| A231 | | C | B |
| A232 | | D | C |
| A233 | | C | B |
| A234 | | D | D |
| A235 | | D | D |
| A236 | | D | D |
| A237 | | B | B |
| A238 | | A | B |
| A239 | | A | B |
| A240 | | A | B |
| A241 | | A | B |
| A242 | | A | B |
| A243 | | B | B |
| A244 | | C | B |
| A245 | | C | C |
| A246 | | D | D |
| A247 | | D | D |
| A248 | | D | D |
| A249 | | D | D |
| A250 | | D | D |
| A251 | | D | D |
| A252 | | D | D |
| A253 | | D | D |
| A254 | | D | D |
| A255 | | D | D |
| A256 | | D | D |
| A257 | | D | D |
| A258 | D | | |
| A259 | | D | C |
| A260 | | D | C |
| A261 | | D | D |
| A262 | | A | B |
| A263 | | A | B |
| A264 | | A | B |
| A265 | | A | B |
| A266 | | A | B |
| A267 | | A | B |
| A268 | | A | B |
| A269 | | B | B |
| A270 | | A | B |
| A271 | | D | D |
| A272 | | B | B |
| A273 | | B | B |
| A274 | | B | B |
| A275 | | D | C |
| A276 | | A | C |
| A277 | | A | A |
| A278 | | A | A |
| A279 | | A | B |
| A280 | | A | A |
| A281 | | A | A |
| A282 | | A | B |
| A283 | | B | B |
| A284 | | A | B |
| A285 | | A | B |
| A286 | | B | B |
| A287 | | A | B |
| A288 | | A | B |
| A289 | | A | B |
| A290 | | A | B |
| A291 | | A | B |
| A292 | | A | B |
| A293 | | A | B |
| A294 | | A | B |
| A295 | | A | B |
| A296 | | A | B |
| A297 | | A | B |
| A298 | | B | B |
| A299 | | A | B |
| A300 | | A | B |
| A301 | | A | B |
| A302 | | A | B |
| A303 | | C | B |
| A304 | | D | C |
| A305 | | D | C |
| A306 | D | | |
| A307 | D | | |
| A308 | | D | D |
| A309 | | D | C |
| A310 | | D | D |
| A311 | | D | C |
| A312 | | D | C |
| A313 | | B | B |
| A314 | | A | B |
| A315 | | B | C |
| A316 | | D | C |
| A317 | | C | C |
| A318 | | A | B |
| A319 | | D | D |
| A320 | | D | C |
| A321 | | D | D |
| A322 | | D | D |
| A323 | | D | D |
| A324 | | D | C |
| A325 | | D | D |
| A326 | | D | C |
| A327 | | B | B |
| A328 | | A | B |
| A329 | | B | B |
| A330 | | A | B |
| A331 | | D | D |
| A332 | | D | D |
| A333 | | D | C |
| A334 | | D | C |
| A335 | | D | D |
| A336 | | D | C |
| A337 | | B | B |
| A338 | | D | D |
| A339 | | D | C |
| A340 | | D | C |
| A341 | | D | D |
| A342 | | D | D |
| A343 | | D | C |
| A344 | | D | C |
| A345 | | D | D |
| A346 | | A | B |
| A347 | | A | B |
| A348 | | A | B |
| A349 | | A | B |
| A350 | | A | B |
| A351 | | D | C |
| A352 | | C | D |
| A353 | | D | C |
| A354 | | D | C |
| A355 | | C | C |
| A356 | | D | C |
| A357 | | A | B |
| A358 | | D | C |
| A359 | | A | B |
| A360 | | A | B |
| A361 | | D | C |
| A362 | | D | C |

IC₅₀: “A”: <=0.01 uM; “B”: >0.01 and <=0.1 uM; “C”: >0.1 and <=1 uM; and “D”: >1 uM
DC₅₀: “A”: <=50 nM; “B”: >50 and <=200 nM; “C”: >200 and <=500 nM; and “D” >500 nM.
Dₘₐₓ: “A” >=80%; “B” >=60% and <80%; “C” >=40% and <60%; and “D” <40%

### For Compound B-1 to B-58

### In Vitro Assay: IC₅₀ Measurements for Binding to CRBN/DDBI

The binding potency was determined using HTRF assay technology (Perkin Elmer). Compounds were serially diluted in DMSO and 0.2 μL volume was transferred to white 384-well plate. The reaction was conducted in total volume of 20 μL with addition of 2 nM His tagged CRBN+DDB−DLS7+CXU4 (Wuxi, catalogue #RP210521GA) to compounds followed by addition of 60 nM Fluorescent probe Cy5-labeled Thalidomide (Tenova Pharma, catalogue #T52461), and 0.4 nM of MAb Anti-61HIS Tb cryptate Gold (Cisbio, catalogue #61HI2TLA in the assay buffer (50 mM HEPES pH 7.5, 1 mM TCEP, 0.01% Brij-35, 50 mM NaCl, and 0.1% BSA). After one hour incubation at room temperature, the HTRF signals were read on Envision reader (Perkin Elemer). Data was analyzed using XLfit using four parameters dose response curve to determine IC₅₀s. Results are summarized in Table E3.

TABLE E3
CRBN binding activity

| | CRBN |
|---|---|
| | Binding activity |
| Example | IC₅₀ |
| B1 | C |
| B2 | D |
| B3 | A |
| B4 | C |
| B5 | B |
| B7 | B |
| B8 | B |
| B9 | A |
| B10 | B |
| B11 | A |
| B12 | B |
| B13 | B |
| B14 | D |
| B16 | B |
| B17 | C |
| B18 | B |
| B19 | D |
| B20 | C |
| B21 | B |
| B22 | B |
| B23 | C |
| B28 | B |
| B29 | A |
| B30 | D |
| B31 | B |
| B32 | B |
| B33 | B |
| B34 | B |
| B35 | A |
| B36 | B |
| B37 | A |
| B38 | B |
| B39 | B |
| B40 | B |
| B41 | B |
| B42 | B |
| B43 | D |
| B44 | A |
| B45 | B |
| B48 | B |
| B49 | B |
| B50 | B |
| B51 | A |
| B52 | B |

A: IC₅₀ < 100 nM; B: 100 nM < IC₅₀ < 500 nM; C: 500 nM < IC₅₀ < 1000 nM; D: 1000 nM < IC₅₀ < 5000 nM; E: 5000 nM < IC₅₀ < 10000 nM

### In Vitro Assay: IKZF2 FACS Assay

Jurkat cells (ATCC, Cat #HB-8065) were cultured in RPMI1640+10% FBS+1% P/S. Cells were treated at desired compound concentrations (0.05 to 10 μM) and DMSO as vehicle control for 24 hrs. After 24 hrs of drug treatment cells were washed, fixed (3.7% PFA, and permeabilized with perm buffer (0.3% Triton X-100 in 1% BSA Solution). Subsequently, cells were stained with IKZF2 (1:100, Cell signaling) primary antibody and Alexa 488-labelend anti-rabbit IgG (1:200, Cell Signaling) secondary antibodies in staining buffer (1% BSA in PBS). Cells were images on iQue Flowcytometer and IKZF2 levels were quantified using iQue software. Data was further analyzed using XLfit using four parameters dose response curve to determine DC₅₀ and Dₘₐₓ. The half maximal degradation concentration values (DC₅₀) and maximal degradation percentage (Dₘₐₓ, %) of IKZF2 are summarized in Table E4.

TABLE E4
IKZF2 degradation activity by FACS

IKZF2 FACS degradation

| Example | DC₅₀ | Dₘₐₓ |
|---|---|---|
| B14 | B | B |
| B26 | A | A |
| B27 | B | C |
| B32 | A | B |
| B46 | A | B |
| B51 | A | A |
| B53 | A | B |
| B55 | A | B |

DC₅₀, A: <10 nM; B: >10 nM.
Dₘₐₓ, A: >60%; B: 40-60%; C: <40%

### In Vitro Assay: IKZF2 HiBit Assay PGP-138.11

The HiBiT protein tagging system was applied to modified HEK293T Flp-in-HiBiT cells (polyclone) via a CRISPR/Cas9—mediated insertion of the HiBiT peptide tag (Promega™) to the N-terminus of the IKZF2 gene locus (Neon™ transfection system). Test and reference compounds are diluted from 1 mM at 3 folds for 11 doses. 25 nL of diluted compound is transferred to assay plates (Corning3570) using ECHO550, the final DMSO concentration @0.1%. The cells are seeded in 3000/25 mL/well to compound plates. It is then incubated for 6 hrs in TC incubator. The amount of Nano-Glo® HiBiT lytic reagent needed to perform the desired experiments is calculated. The Nano-Glo® HiBiT lytic reagent is brought to room temperature. The LgBiT protein is diluted to 1:100 and the Nano-Glo® HiBiT lytic substrate is diluted to 1:50 into an appropriate volume of room temperature Nano-Glo® HiBiT lytic buffer. 15 mL of the detection reagent (or without LgBiT) is dispensed to corresponding well according to the layout. The plate is then shaked for 10 mins at room temperature. After briefly centrifuge, the plate is read on Envision. At the indicated timepoints, the Nano-Glo® HiBiT lytic detection system (Promega™) was utilized for detecting bioluminescence of the HiBiT tag in treated cells: abundance of the tag is proportionate to the level of luminescence. Following normalization to DMSO, dose-response curves were plotted (GraphPad Prism) to determine the concentration points at which 50% of HiBiT-Helios degradation was achieved by each compound. The extent of degradation (range of luminescence) from the highest to lowest concentration points was calculated to determine the Dₘₐₓ. Results of IKZF2 degradation activity are shown in Table E5.

TABLE E5
IKZF2 degradation activity by HiBit

IKZF2 HiBit degradation

| Example | DC₅₀ | Dₘₐₓ |
|---|---|---|
| B1 | B | C |
| B2 | B | B |
| B3 | C | B |
| B4 | B | B |
| BS | B | B |
| B6 | B | B |
| B7 | B | B |
| B8 | B | B |
| B9 | B | B |
| B10 | B | B |
| B11 | B | A |
| B12 | B | A |
| B13 | B | B |
| B14 | B | A |
| B15 | B | A |
| B16 | B | C |
| B17 | E | E |
| B18 | E | E |
| B19 | D | C |
| B20 | E | D |
| B21 | B | D |
| B22 | C | D |
| B23 | E | E |
| B24 | C | B |
| B25 | B | B |
| B26 | B | A |
| B27 | B | B |
| B28 | B | B |
| B29 | A | A |
| B30 | B | B |
| B31 | B | B |
| B32 | A | B |
| B33 | B | B |
| B34 | B | B |
| B35 | B | B |
| B36 | B | B |
| B37 | B | B |
| B38 | B | B |
| B39 | A | B |
| B40 | B | B |
| B41 | B | B |
| B42 | B | B |
| B43 | B | B |
| B44 | B | A |
| B45 | B | A |
| B46 | B | B |
| B47 | C | B |
| B48 | A | B |
| B49 | A | A |
| B50 | B | B |
| B51 | A | B |
| B52 | B | C |
| B53 | A | A |
| B54 | B | B |
| B55 | A | A |
| B56 | C | C |
| B57 | C | B |
| B58 | B | C |

DC₅₀, A: <1 nM; B: 1-10 nM; C: 10-100 nM; D: 100-1000 nM; E: >1000 nM.
Dₘₐₓ, A: >80%; B: 60-80%; C: 40-60%; D: 20-40%; E: <20%.

### In Vitro Assay: IKZF1 HiBit Assay

The HiBiT protein tagging system was applied to modified Cells: modified HEK293T Flp-in-HiBiT-IKZF1 stable cell line (polyclone) via a CRISPR/Cas9—mediated insertion of the HiBiT peptide tag (Promega™) to the N-terminus of the IKZF2 gene locus (Neon™ transfection system).

Test compound from 10 mM and reference compound (CC-92480 from 50 mM and I-57 from 10 mM) are diluted at 3 folds for 11 doses. 25 nL of diluted compound is transferred to assay plates (Corning3571) using ECHO550, the final DMSO concentration @0.1%. The cells are seeded in 3000/25 mL/well to compound plates. The plates are incubated for 6 hrs in TC incubator. The amount of Nano-Glo® HiBiT lytic reagent needed to perform the desired experiments is calculated. The Nano-Glo® HiBiT lytic reagent is brought to room temperature. The LgBiT protein is diluted to 1:100 and the Nano-Glo® HiBiT lytic substrate is brought to 1:50 into an appropriate volume of room temperature Nano-Glo® HiBiT lytic buffer. 15 mL of the detection reagent (or without LgBiT) is dispensed to corresponding well according to the layout. The plate is shaked for 10 mins at room temperature. After briefly centrifuging, the plate is read on Envision. At the indicated timepoints, the Nano-Glo® HiBiT lytic detection system (Promega™) was utilized for detecting bioluminescence of the HiBiT tag in treated cells: abundance of the tag is proportionate to the level of luminescence. Following normalization to DMSO, dose-response curves were plotted (GraphPad Prism) to determine the concentration points at which 50% of HiBiT-Ikaros degradation was achieved by each compound. The extent of degradation (range of luminescence) from the highest to lowest concentration points was calculated to determine the Dm. Results of IKZF1 degradation activity are shown in Table E6.

TABLE E6
IKZF1 degradation activity by HiBit

IKZF1 HiBit degradation

| Example | DC₅₀ | Dₘₐₓ |
|---|---|---|
| B1 | D | B |
| B2 | C | B |
| B3 | E | C |
| B4 | E | C |
| B5 | E | C |
| B6 | E | C |
| B7 | E | C |
| B8 | E | C |
| B9 | E | C |
| B10 | E | C |
| B11 | E | C |
| B12 | E | C |
| B13 | E | C |
| B14 | E | C |
| B15 | E | C |
| B16 | E | C |
| B17 | E | C |
| B18 | E | C |
| B19 | E | C |
| B20 | E | C |
| B21 | E | C |
| B22 | E | C |
| B23 | E | C |
| B24 | E | C |
| B25 | B | B |
| B26 | E | C |
| B27 | E | C |
| B28 | E | C |
| B29 | B | A |
| B30 | E | C |
| B31 | E | C |
| B32 | E | C |
| B33 | E | C |
| B34 | E | C |
| B35 | E | C |
| B36 | A | B |
| B37 | E | C |
| B38 | E | C |
| B39 | E | C |
| B40 | E | C |
| B41 | C | B |
| B42 | E | C |
| B43 | E | C |
| B44 | D | B |
| B45 | C | B |
| B46 | E | C |
| B47 | E | C |
| B48 | E | C |
| B49 | E | C |
| B50 | E | C |
| B51 | E | C |
| B52 | E | C |
| B53 | E | C |
| B54 | E | B |
| B-55 | E | C |
| B-56 | E | C |
| B-57 | E | C |
| B-58 | E | C |

DC₅₀, A: <1 nM; B: 1-10 nM; C: 10-100 nM; D: 100-1000 nM; E: >1,000 nM.
Dmₐₓ, A: >40%; B: 20-40%; C: <20%.

### IKZF2 Degradation and Evaluation of IL-2 Production

IKZF2 is important for immunosuppressive activity of regulatory T cells (T_{reg} cells), which is linked to interleukin-2 (IL-2) repression. IKZF2 binds to the IL-2 promoter in T_{reg} cells and suppresses transcriptional activation. IKZF2 knockdown suppresses FoxP3 binding to IL-2 promoter and results in higher IL-2 expression upon stimulation. Further, IKZF2 knockout leads to an unstable CD4 T_{reg} phenotype in mice marked by production of effector cytokines and IKZF2 knockout in T_{regs} suppresses tumor growth. (Baine I. et al., J Immunol 190, 1008-1016 (2013); Nakagawa, H. et al. Proc National Acad Sci 113, 6248-6253 (2016); Yates, K., et al. Proc National Acad Sci 115, 201720447 (2018).

To measure whether IKZF2 degradation with the compounds of this disclosure impacts IL-2 production, Jurkat cells (ATCC, Cat #HB-8065) are treated with vehicle control (DMSO) or the compound for 16-24 hrs. After 16-24 hrs of treatment cells are stimulated with CD3/CD28 stimulation beads at a 3:1 ratio for 24 hrs. After 24 hrs, supernatants are collected and the concentration of IL-2 is measured using MSD V-PLEX Human IL-2 Kit (Cat #K151QQD, Mesoscale). The compounds of this disclosure are expected to increase IL-2 production, and thereby increase anti-tumor immunity.

### IKZF2 Degradation in Primary Human T_{reg} Cells

To measure whether the compounds of this disclosure can induce degradation of IKZF2 in T_{reg} cells, human peripheral bone marrow cells (PBMCs) obtained from healthy donors purchased from Milestone Biological Science and Technology Company are treated with vehicle control (DMSO) or the compound for various time points (3-24 hrs). After desired treatment time, the cells are collected and stained with anti-CD3-APC-Cy7 (Clone SP34-2, BD), anti-CD4-FITC (Clone L200, BD), anti-CD45-BV510 (Clone HI30, Biolegend), and anti-CD25-BV421 (Clone BC96, Biolegend) in cell staining buffer (Biolegend, Cat #420201), washed and fixed with FOXP3 fix/perm buffer (Life Technologies, cat. #00-5523-00) followed by intracellular staining with anti-IKZF2-APC (Clone 22F6, BioLegend), anti-Ikaros-PE-Cy7 (Clone 16B5C71, BioLegend), and anti-FOXP3-PE (clone 206D, Biolegend). Samples are acquired on a Thermo Attune N×T flow cytometer (Thermo Fisher Scientific). IKZF2 mean fluorescence intensity (MFI) and IKZF1 MFI are measured in T_{regs} (CD4+CD25+FOXP3+) cells. The compounds of this disclosure are expected to degrade IKZF2 in T_{reg} cells, thereby suppressing the action of T_{reg} cells.

### IKZF2 Degradation and T_{eff} Cell Proliferation

To measure whether the compounds of this disclosure can enhance effector T cell (T_{eff}) proliferation via suppression of T_{reg} cells, T_{reg} cells and T_{eff} cells from matched human donors are co-cultured in the presence of vehicle control (DMSO) or compound. T_{reg} cells are isolated from human peripheral bone marrow cells (PBMCs) obtained from healthy donors purchased from Milestone Biological Science and Technology Company. CD4 enrichment by negative selection followed by CD25 enrichment by positive selection are performed using the human CD4 T cell isolation kit (cat. #130-096-533) and human CD25 microbeads (cat. #130-092-983) from Miltenyi Biotec (Cambridge, MA) according to manufacturer's instructions. Isolated T_{regs} are expanded for 8-14 days in the presence of compound or DMSO, using T_{reg} expander beads (ThermoFisher, cat. #11129D) or T-cell activator beads (ThermoFisher, cat. #11161D) at a 4:1 or 3:1 ratio, respectively, in the presence of 500 U/mL rhIL-2. Expanded T_{reg} cells are dispensed in co-culture with carboxyfluorescein succinimidyl ester (CFSE)-labelled CD3+ T-Cells from the matched donor at various T_{reg}:CD3+ T cell ratios in the presence of T-cell activator beads or soluble anti-CD3 antibody (30 ng/mL, OKT3, Thermofisher cat. #16-0037-81). After 3-5 days of incubation, proliferation of CD8+ T_{eff} cells is assessed by analyzing CFSE dye dilution in CD8+ T-Cells (anti-CD8-PerCP/Cyanine5.5, clone SK1, Biolegend) using flow cytometry. Analysis is performed using a Thermo Attune N×T flow cytometer (Thermo Fisher Scientific). T_{eff} cells that proliferate during the co-culture are identified as having diluted CFSE and data are plotted as the proportion of CFSE low, proliferated cells in the final culture. The compounds of this disclosure are expected to suppress T_{reg} cells, thereby enhancing T_{eff} cell proliferation.

### In Vivo Pharmacology and Efficacy Studies

### Cynomolgus Moneys

To determine in vivo efficacy of the compounds of this disclosure, non naïve cynomolgus monkeys are treated with a single oral dose of vehicle or the compound. Whole blood from the treated monkeys is collected across time (e.g., various timepoints between 0 hr-96 hrs) and stained with anti-CD3-APC-Cy7 (Clone SP34-2, BD), anti-CD4-FITC (Clone L200, BD), anti-CD45-BV786 (Clone D058-1283, Biolegend), and anti-CD25-APC (Clone BC96, Biolegend) in cell staining buffer (Biolegend, Cat #420201), washed and fixed with FOXP3 fix/perm buffer (Life Technologies, cat. #00-5523-00) followed by intracellular staining with anti-IKZF2-PE (Clone 22F6, BioLegend) and anti-FOXP3-BV421 (clone 206D, Biolegend). Samples are acquired on a Thermo Attune N×T flow cytometer (Thermo Fisher Scientific). IKZF2 mean fluorescence intensity (MFI) is measured in T_{regs} (CD4+CD25+FOXP3+) cells. The compounds of this disclosure are expected to suppress IKZF2+ T_{regs} in cynomolgus monkeys.

### Mice

To determine in vivo efficacy of the compounds of this disclosure, CRBN^{I391V} mice are treated with a single oral dose of vehicle or the compound. CRBN^{I391V} mice are used because a single amino acid difference within the CRBN-Immunomodulatory drug (IMiD) binding region renders mouse CRBN resistant to degradation by IMiDs. A change from Ile 391 to Val in mouse CRBN restores IMiD-induced degradation of IKZF3. Fink, E. C. et al. Blood 132, 1535-1544 (2018); Gemechu, Y. et al. P Natl Acad Sci Usa 115, 11802-11807 (2018).
1. IKZF2 degradation in mice: Various doses of the vehicle and compound are tested in the mice and analyzed across time (e.g., various timepoints between 0 hr-12 hrs) and analyzed using western blot assay to measure the percentage of IKZF2 remaining in tissues (e.g., spleen and thymus). Tissue is lysed in RIPA buffer (Cell Signaling, cat #9806) containing Halt™ protease/phosphatase inhibitor cocktail (Thermo, Cat #78440). After assessing protein concentration by BCA assay (Pierce), equal amounts of protein for each sample are loaded into 4-12% Bis-Tris gels (Invitrogen), transferred to nitrocellulose membranes and immunoblotted with antibodies against Helios (Cell Signaling, Cat #4247) and b-Actin (Cell Signaling, Cat #3700). Membranes are developed on an Odyssey detection system (LI-COR Biosciences) after incubation with IRDye800-labeled goat anti-rabbit IgG and IRDye680-labeled goat anti-mouse IgG (LI-COR) secondary antibodies. The compounds of this disclosure are expected to degrade IKZF2 in CRBN^{I391V} mice.
2. Tumor growth inhibition in mice: To develop cancer cell line xenografts, CRBN^{I391V} mice are implanted with MC38 cells (ATCC) subcutaneously to induce tumor formation. MC38 cells (e.g., five million) in 50% Matrigel are injected subcutaneously into CRBN^{I391V} mice to induce tumor formation. Mice are treated with vehicle control (e.g., 5% DMSO, 10% solutol, 85% Water) or the compound once tumors reach ˜80-400 mm³, and sacrificed when tumor volume reached 2000 mm³ or at the end of the study (whichever occurs first). Tumor sizes and animal weights are measured 2-3 times per week. Tumor volume (mm³)=(length×width²)/2. Tumor growth inhibition is calculated using TGI (%)=(1−((Tₑ−T₀)/(Cₑ−C₀)))′ 100, where Tₑ=Test tumor volume endpoint, T₀=Test tumor volume at start of dosing, Cₑ=Vehicle control tumor volume endpoint, C₀=Vehicle control tumor volume at start of dosing The compounds of this disclosure are expected to inhibit MC38 tumor growth in CRBN^{I391V} mice.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula (I′): 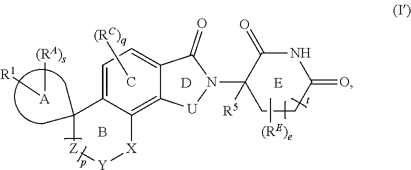
or a pharmaceutically acceptable salt, solvate, or stereoisomer thereof, wherein:
X is —C(R³)₂—, —NR⁴—, —O—, —S—, —S(═O)—, or —S(═O)₂—;
Y is —C(R³)₂—, —NR⁴—, —O—, —S—, —S(═O)—, or —S(═O)₂—;
each Z is independently —C(R³)₂—, —NR⁴—, —O—, —S—, —S(═O)—, or —S(═O)₂—;
p is 0, 1, or 2;
each R³ is independently deuterium, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, aryl, heteroaryl, carbocyclyl, or heterocyclyl is optionally substituted with one or more R^{u};
two geminal R³ together form oxo; or
two geminal R³, together with the carbon atom to which they are attached, form C₃₋₆ carbocyclyl or 3- to 6-membered heterocyclyl, wherein the carbocyclyl or heterocyclyl is optionally substituted with one or more R^{u};
each R⁴ is independently hydrogen or C₁₋₆ alkyl optionally substituted with one or more R^{u};
Ring A is C₃₋₁₂ carbocycle or 3- to 12-membered heterocycle;
R¹ is hydrogen or -M-L-Q-R²;
M is absent, —(C═O)—, —S(═O)—, or —S(═O)₂—;
L is absent or [W]ᵣ;
r is an integer from 1 to 3;
each W is independently —C(R^{L})₂—, C₃₋₄ carbocyclylene, or 3- to 4-membered heterocyclylene, wherein the carbocyclylene or heterocyclylene is optionally substituted with one or more R^{u};
each R^{L} is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}; or
two geminal R^{L}, together with the carbon atom to which they are attached, form C₃₋₆ carbocyclyl or 3- to 6-membered heterocyclyl, wherein the carbocyclyl or heterocyclyl is optionally substituted with one or more R^{u};
Q is absent, —NR^{Q}—, —O—, —C(═O)—, —S(═O)—, or —S(═O)₂—;
R^{Q} is hydrogen or C₁₋₆ alkyl optionally substituted with one or more R^{u};
R² is C₆₋₁₀ aryl, 5- to 10-membered heteroaryl, C₃₋₁₂ carbocyclyl, or 3- to 12-membered heterocyclyl, wherein the aryl, heteroaryl, carbocyclyl, or heterocyclyl is optionally substituted with one or more R^{2a};
each R^{2a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkylene, alkoxy, alkylamino, alkenyl, alkynyl, aryl, heteroaryl, carbocyclyl, or heterocyclyl is optionally substituted with one or more R^{u}; or
two R^{2a}, together with the atoms to which they are bonded, form C₃₋₈ carbocyclyl or 3- to 8-membered heterocyclyl, wherein the carbocyclyl or heterocyclyl is optionally substituted with one or more R^{u};
each occurrence of R^{A}, R^{C}, and R^{E} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
q is an integer from 0 to 2;
s is an integer from 0 to 12, as valency permits;
e is an integer selected from 0 to 5;
U is —CH₂— or —C(═O)—;
R⁵ is hydrogen, deuterium, C₁₋₆ haloalkyl, or C₁₋₆ alkyl; and
t is an integer from 0 to 2;
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkylene, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, and 3- to 12-membered heterocyclyl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
R^{c} and R^{d} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl, wherein the heterocyclyl is optionally substituted with one or more R^{z}, wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

2. The compound of claim 1, wherein when p is 0, then X and Y are not both —C(R³)₂; and when p is 1, then X, Y, and Z are not all —C(R³)₂.

3. The compound of claim 1, wherein X is —O— and Y is —C(R³)₂—.

4. The compound of claim 1, wherein X is —C(R³)₂— and Y is —O—.

5. The compound of claim 1, wherein X is —NR⁴— and Y is —C(R³)₂—.

6. The compound of any one of claims 1-5, wherein p is 0 or 1.

7. The compound of claim 1, wherein the compound is a compound of Formula (I′-1-ii), (I′-1-iii)(I′-1-iv), (I′-1-v), (I′-1-vi), (I′-1-vii), (I′-1-ix), or (I′-1-xii): 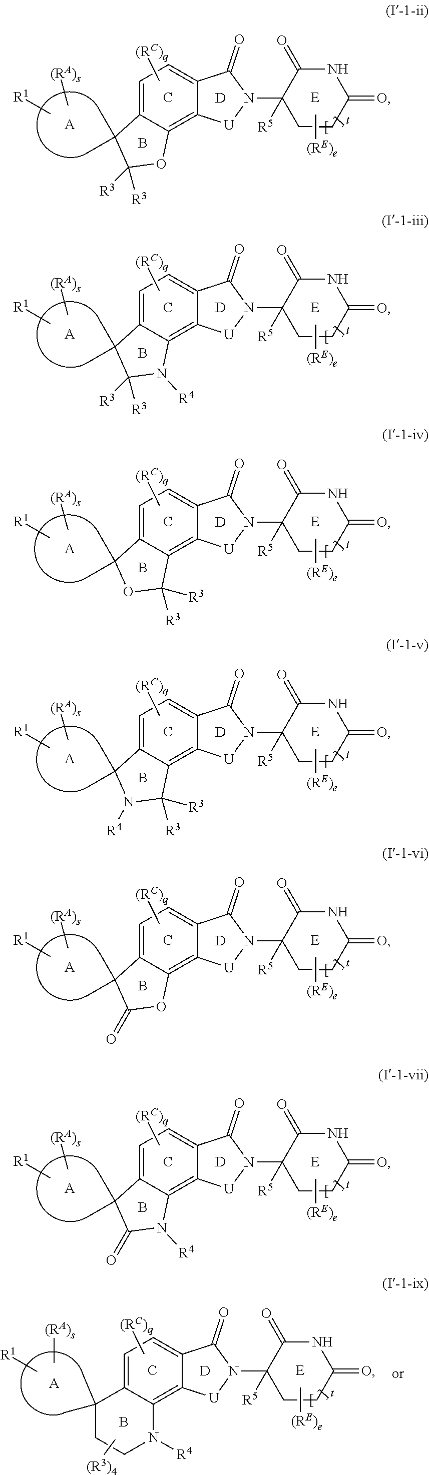 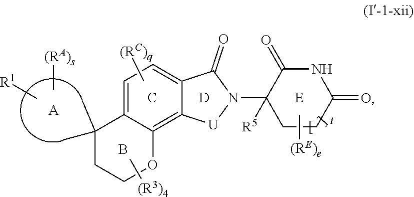
or a pharmaceutically acceptable salt, solvate, or stereoisomer thereof.

8. The compound of any one of claims 1-7, wherein Ring A is 3- to 12-membered heterocycle.

9. The compound of any one of claims 1-8, wherein 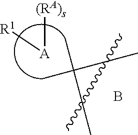
is 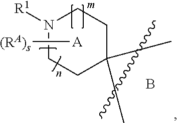
wherein m and n are independently an integer from 0 to 2.

10. The compound of claim 9, wherein the compound is a compound of Formula (I′-2-ii), (I′-2-iii), (I′-2-iv), (I′-2-v), (I′-2-vi), (I′-2-vii), (I′-2-ix), or (I′-2-xii) 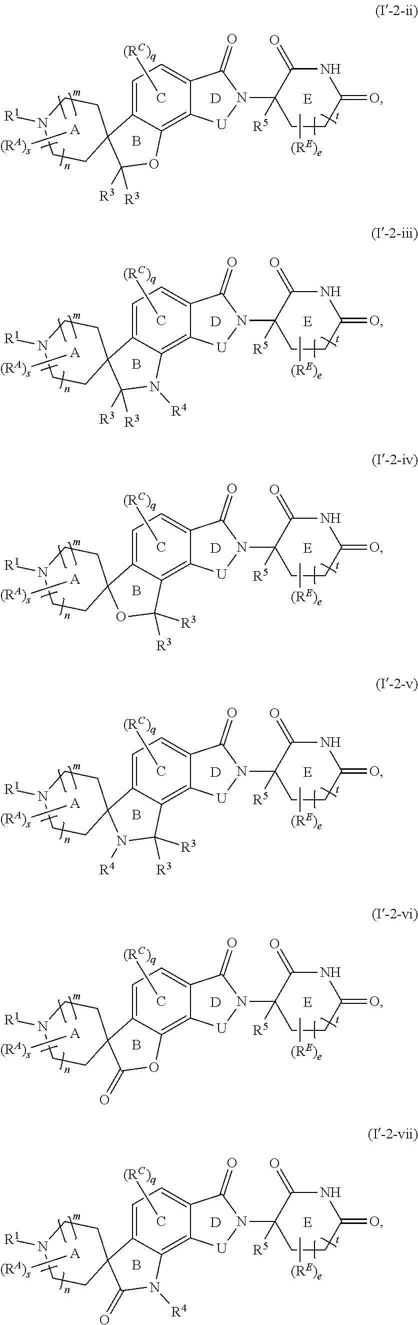 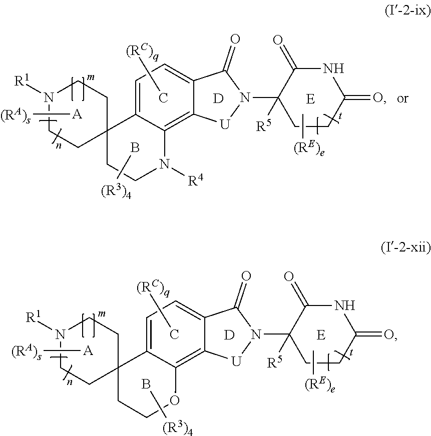
or a pharmaceutically acceptable salt, solvate, or stereoisomer thereof.

11. The compound of claim 9 or 10, wherein each of m and n is 1.

12. The compound of any one of claims 1-11, wherein R¹ is hydrogen.

13. The compound of any one of claims 1-11, wherein R¹ is -L-R².

14. The compound of claim 13, wherein L is —C(R^{L})₂—.

15. The compound of claim 14, wherein each R^{L} is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

16. The compound of claim 14, wherein each R^{L} is independently hydrogen, deuterium, or C₁₋₆ alkyl.

17. The compound of claim 14, wherein L is —CH₂—.

18. The compound of any one of claims 13-17, wherein R² is C₆₋₁₀ aryl or 5- to 10-membered heteroaryl, C₃₋₁₂ carbocyclyl, or 3- to 12-membered heterocyclyl, wherein the aryl, heteroaryl, carbocyclyl, or heterocyclyl is optionally substituted with one or more R^{2a}.

19. The compound of claim 18, wherein R² is phenyl optionally substituted with one or more R^{2a}.

20. The compound of claim 18, wherein R² is 5- to 10-membered heteroaryl optionally substituted with one or more R^{2a}.

21. The compound of claim 18, wherein R² is C₅₋₁₀ carbocyclyl optionally substituted with one or more R^{2a}.

22. The compound of claim 18, wherein R² is 9- to 10-membered heterocyclyl optionally substituted with one or more R^{2a}.

23. The compound of any one of claims 19-22, wherein each R^{2a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

24. The compound of any one of claims 19-22, wherein each R^{2a} is independently oxo, halogen, —CN, —OH, C₁₋₆ alkyl, C₁₋₆ alkoxy, C₂₋₆ alkylamino, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂NR^{c}R^{d}, —NR^{c}S(═O)₂R^{a}, —NR^{b}C(═O)R^{a}, —C(═O)OR^{b}, or —C(═O)NR^{c}R^{d}, wherein the alkyl, alkylene, alkoxy, alkylamino, carbocyclyl, heterocyclyl, aryl, or heteroaryl is optionally substituted with one or more R^{u}.

25. The compound of any one of claims 1-24, wherein each R³ is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

26. The compound of any one of claims 1-24, wherein each R³ is independently hydrogen, deuterium, or C₁₋₆ alkyl.

27. The compound of any one of claims 1-26, wherein each R⁴ is independently hydrogen or C₁₋₆ alkyl.

28. The compound of any one of claims 1-27, wherein each occurrence of R^{A}, R^{C}, and R^{E} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

29. The compound of claim 28, wherein each R^{A} is independently halogen, —OH, or C₁₋₆ alkyl.

30. The compound of claim 28, wherein each R^{C} is independently halogen, —CN, or C₁₋₆ alkyl.

31. The compound of any one of claims 1-30, wherein e is 0.

32. The compound of any one of claims 1-31, wherein U is —CH₂—.

33. The compound of any one of claims 1-32, wherein R⁵ is hydrogen.

34. The compound of any one of claims 1-33, wherein t is 1.

35. The compound of claim 1, wherein the compound is a compound of Formula (I′): 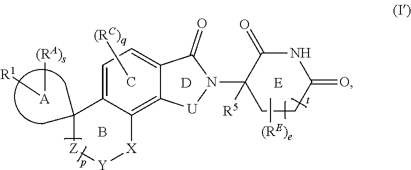
or a pharmaceutically acceptable salt, solvate, or stereoisomer thereof, wherein:
R¹ is hydrogen or -L-R²;
L is —C(R^{L})₂—;
each R^{L} is independently hydrogen, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u};
R² is C₆₋₁₀ aryl, 5- to 10-membered heteroaryl, C₃₋₁₂ carbocyclyl, or 3- to 12-membered heterocyclyl, wherein the aryl, heteroaryl, carbocyclyl, or heterocyclyl is optionally substituted with one or more R^{2a};
each R^{2a} is independently oxo, halogen, —CN, —OH, C₁₋₆ alkyl, C₁₋₆ alkoxy, C₂₋₆ alkylamino, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂NR^{c}R^{d}, —NR^{c}S(═O)₂R^{a}, —NR^{b}C(═O)R^{a}, —C(═O)OR^{b}, or —C(═O)NR^{c}R^{d}, wherein the alkyl, alkylene, alkoxy, alkylamino, carbocyclyl, heterocyclyl, aryl, or heteroaryl is optionally substituted with one or more R^{u};
each occurrence of R^{A} and R^{C} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u};
e is 0;
X is —O— or —NR⁴—;
each R⁴ is independently hydrogen or C₁₋₆ alkyl;
Y is —CH₂— or —O—; and
p is 0 or 1.

36. The compound of claim 1, wherein the compound is selected from the compounds in Tables 1 and 2 and pharmaceutically acceptable salts thereof.

37. A pharmaceutical composition comprising the compound of any one of claims 1-36, and a pharmaceutically acceptable excipient.

38. A method of degrading an IKZF2 protein in a subject or biological sample comprising administering the compound of any one of claims 1-36 to the subject or contacting the biological sample with the compound of any one of claims 1-36.

39. Use of the compound of any one of claims 1-36 in the manufacture of a medicament for degrading an IKZF2 protein in a subject or biological sample.

40. A compound of any one of claims 1-36 for use in degrading an IKZF2 protein in a subject or biological sample.

41. A method of treating or preventing a disease or disorder a subject in need thereof, comprising administering to the subject the compound of any one of claims 1-36.

42. Use of the compound of any one of claims 1-36 in the manufacture of a medicament for treating or preventing a disease or disorder in a subject in need thereof.

43. A compound of any one of claims 1-36 for use in treating or preventing a disease or disorder in a subject in need thereof.

44. The method, use, or compound of any one of claims 41-43, wherein the disease or disorder is an IKZF2-mediated disease or disorder.

45. The method, use, or compound of any one of claims 41-43, wherein the disease or disorder is T cell leukemia, T cell lymphoma, Hodgkin's lymphoma or non-Hodgkin's lymphoma, myeloid leukemia, non-small cell lung cancer (NSCLC), melanoma, triple-negative breast cancer (TNBC), nasopharyngeal cancer (NPC), microsatellite stable colorectal cancer (mssCRC), thymoma, carcinoid, or gastrointestinal stromal tumor (GIST).
